(19) United States
(12) Patent Application Publication
(10) Pub. No.: US 2025/0359478 A1
(43) Pub. Date: Nov. 20, 2025

(54) COMPOUND AND COMPOSITION AND ORGANIC OPTOELECTRONIC DEVICE AND DISPLAY DEVICE

(71) Applicant: SAMSUNG SDI CO., LTD., Yongin-si (KR)

(72) Inventors: Dongjin JANG, Suwon-si (KR); Eunhye AN, Suwon-si (KR); Giwook KANG, Suwon-si (KR); Eunjeong CHOI, Suwon-si (KR); Soonhyung KWON, Suwon-si (KR); Jisoo SHIN, Suwon-si (KR); Dosik KIM, Suwon-si (KR); Youngkyoung JO, Suwon-si (KR); Ho Kuk JUNG, Suwon-si (KR); Mijin LEE, Suwon-si (KR)

(21) Appl. No.: 19/188,250

(22) Filed: Apr. 24, 2025

(30) Foreign Application Priority Data
May 17, 2024 (KR) 10-2024-0064779

(51) Int. Cl. H10K 85/60; C07D 403/04; C07D 403/10; C07D 403/14; C07D 405/14; C07D 409/14; C07D 491/048; C07D 493/04; C07D 495/04; C09K 11/02; C09K 11/06; H10K 50/12; H10K 85/30; H10K 85/40

(52) U.S. Cl. CPC H10K 85/657; C07D 403/04; C07D 403/10; C07D 403/14; C07D 405/14; C07D 409/14; C07D 491/048; C07D 493/04; C07D 495/04; C09K 11/02; C09K 11/06; H10K 85/346; H10K 85/40; H10K 85/654; H10K 85/6572; H10K 85/6574; H10K 85/658; C09K 2211/1007; C09K 2211/1018; H10K 50/121

(57) ABSTRACT

A compound represented by Chemical Formula 1,

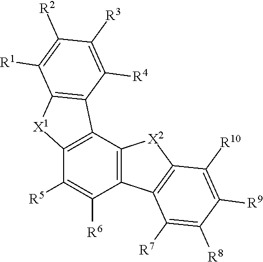

wherein, X¹ and X² are each independently NR^{a}, O, S, Se, or Te, any one of R¹ to R¹⁰ is a group represented by Chemical Formula A, the remainder of R¹ to R¹⁰ and R^{a} are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and R¹ to R¹⁰ and R^{a} are each independently present, or two adjacent ones among R¹ to R¹⁰ and R^{a} are linked to form a ring.

### CROSS-REFERENCE TO RELATED APPLICATION

This application claims priority to and the benefit of Korean Patent Application No. 10-2024-0064779, filed in the Korean Intellectual Property Office on May 17, 2024, the entire contents of which are incorporated herein by reference.

### BACKGROUND

### 1. Field

A compound, a composition, an organic optoelectronic device, and a display device are disclosed.

### 2. Description of the Related Art

An organic optoelectronic device (e.g., an organic optoelectronic diode) is a device capable of converting electrical energy and optical energy to each other. Organic optoelectronic devices may be largely divided into two types according to a principle of operation. One type of the optoelectronic devices is a photoelectric device that generates electrical energy by separating excitons formed by light energy into electrons and holes, and transferring the electrons and holes to different electrodes, respectively. Another type of the optoelectronic devices is a light emitting device that generates light energy from electrical energy by supplying voltage or current to the electrodes.

Examples of the organic optoelectronic devices include an organic photoelectric device, an organic light emitting diode, an organic solar cell, and an organic photoconductor drum. Among them, organic light emitting diodes (OLEDs) are attracting much attention in recent years due to increasing demands for flat panel display devices. The OLED is a device that converts electrical energy into light, and the performance of the OLED is greatly influenced by an organic material between electrodes.

### SUMMARY

According to some example embodiments, a compound represented by Chemical Formula 1 is provided.

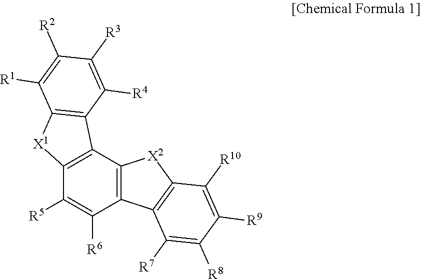

In Chemical Formula 1, X¹ and X² are each independently NR^{a}, O, S, Se, or Te, any one of R¹ to R¹⁰ is a group represented by Chemical Formula A, the remainder of R¹ to R¹⁰ and R^{a} are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and R¹ to R¹⁰ and R^{a} are each independently present, or two adjacent ones among R¹ to R¹⁰ and R^{a} are linked to form a ring,

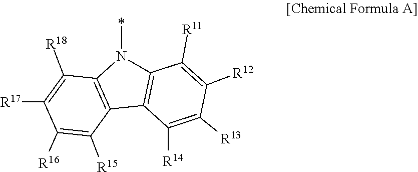

wherein, in Chemical Formula A, R¹¹ to R¹⁸ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, provided that, any one of R¹¹ to R¹⁸ is a substituted or unsubstituted carbazolyl group, R¹¹ to R¹⁸ are each independently present, or two adjacent ones among R¹¹ to R¹⁸ are linked to form a ring, and * is a linking point with Chemical Formula 1.

According to some example embodiments, a composition for an organic optoelectronic device including a first compound, which is the compound, and a second compound represented by Chemical Formula 2.

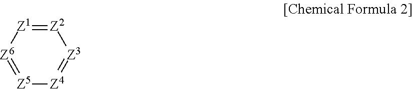

In Chemical Formula 2, Z¹ to Z⁶ are each independently N or C-L^{a}-R^{e}, at least two of Z¹ to Z⁶ are N, L^{a} is each independently a single bond, a substituted or unsubstituted C6 to C20 arylene group, a substituted or unsubstituted divalent C2 to C20 heterocyclic group, or a combination thereof, R^{e} is each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, and R^{e} is each independently present, or two adjacent ones among Res are linked to form a substituted or unsubstituted aliphatic, aromatic or heteroaromatic monocyclic or polycyclic ring.

According to some example embodiments, an organic optoelectronic device includes an anode and a cathode facing each other, and a light emitting layer between the anode and the cathode, wherein the light emitting layer includes the compound or composition for an organic optoelectronic device.

According to some example embodiments, a display device including the organic optoelectronic device is provided.

### BRIEF DESCRIPTION OF THE DRAWINGS

Features will become apparent to those of skill in the art by describing in detail exemplary embodiments with reference to the attached drawings, in which:

FIG. 1 is a cross-sectional view showing an example of an organic light emitting diode according to some embodiments, and

FIG. 2 is a cross-sectional view showing another example of an organic light emitting diode according to some embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of embodiments, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. For example, the “substituted” may refer to replacement of at least one hydrogen of a substituent or a compound by deuterium, a C1 to C20 alkyl group, a C6 to C30 aryl group, or a cyano group. For example, the “substituted” may refer to replacement of at least one hydrogen of a substituent or a compound by deuterium, a C1 to C5 alkyl group, a C6 to C18 aryl group, or a cyano group. For example, the “substituted” may refer to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (-H)” may include

“deuterium substitution (-D)” or “tritium substitution (-T).” As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, e.g., a phenyl group, a naphthyl group, and the like, two or more hydrocarbon aromatic moieties may be linked by a sigma bond and may be, e.g., a biphenyl group, a terphenyl group, a quaterphenyl group, and the like, and two or more hydrocarbon aromatic moieties are fused directly or indirectly to provide a non-aromatic fused ring, e.g., a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

As an example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Below, a compound according to some example embodiments is described.

A compound according to some example embodiments is represented by Chemical Formula 1.

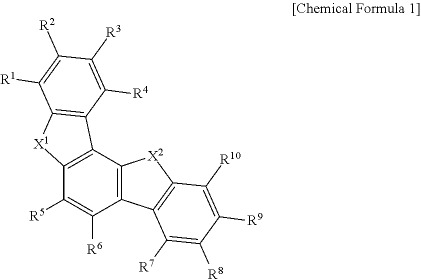

In Chemical Formula 1, X¹ and X² may be each independently NR^{a}, O, S, Se, or Te, any one of R¹ to R¹⁰ may be a group represented by Chemical Formula A, the remainder of R¹ to R¹⁰ and R^{a} may each be independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and R¹ to R¹⁰ and R^{a} may be each independently present, or two adjacent ones among R¹ to R¹⁰ and R^{a} may be linked to form a ring,

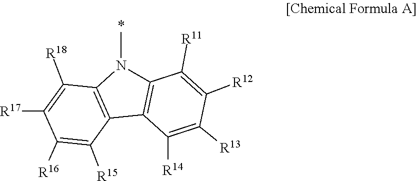

In Chemical Formula A, R¹¹ to R¹⁸ may be each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, provided that, any one of R¹¹ to R¹⁸ may be a substituted or unsubstituted carbazolyl group, R¹¹ to R¹⁸ may be each independently present, or two adjacent ones among R¹¹ to R¹⁸ may be linked to form a ring, and * is a linking point with Chemical Formula 1.

The compound includes a fused ring of a plurality of five-membered rings including heteroatoms and a plurality of benzene rings, and additionally includes a substituted or unsubstituted biscarbazolyl group as a substituent of the benzene rings of such fused rings, thereby increasing the HOMO energy level, increasing the delocalization of electrons, and enhancing the stability of the molecule. Accordingly, the compound may be a compound for organic optoelectronic devices such as organic light emitting diodes, and may exhibit improved life-span characteristics while increasing hole mobility and lowering operating voltage in the organic optoelectronic device.

For example, X¹ and X² in Chemical Formula 1 may be the same.

For example, X¹ and X² in Chemical Formula 1 may be different from each other. For example, one of X¹ and X² may be NR^{a}, and the other of X¹ and X² may be O, S, Se, or Te. For example, one of X¹ and X² may be O or S, and the other of X¹ and X² may be Se or Te. For example, one of X¹ and X² may be O, and the other of X¹ and X² may be S. For example, X¹ and X² may each have NR^{a}, but R^{a} may be different from each other.

For example, at least one of X¹ and X² in Chemical Formula 1 may be NR^{a}, wherein R^{a} may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted benzofuranyl group, a substituted or unsubstituted dibenzofuranyl group, a substituted or unsubstituted benzothiophenyl group, a substituted or unsubstituted dibenzothiophenyl group, a substituted or unsubstituted benzoselenophenyl group, a substituted or unsubstituted dibenzoselenophenyl group, a substituted or unsubstituted benzosilolyl group, or a substituted or unsubstituted dibenzosilolyl group.

For example, any one of R¹ to R⁴ and R⁷ to R¹⁰ of Chemical Formula 1 may be a group represented by Chemical Formula A.

For example, any one of R¹¹ to R¹⁸ of Chemical Formula A may be a group represented by Chemical Formula Aa.

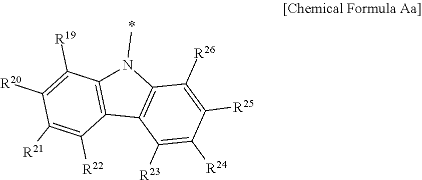

In Chemical Formula Aa, R¹⁹ to R²⁶ may be each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, R¹⁹ to R²⁶ may be each independently present, or two adjacent ones among R¹¹ to R¹⁸ may be linked to form a ring, and * is a linking point with Chemical Formula A.

For example, at least one of R¹⁹ to R²⁶ in Chemical Formula Aa may be deuterium. For example, at least two of R¹⁹ to R²⁶ of Chemical Formula Aa may be deuterium, at least four of R¹⁹ to R²⁶ of Chemical Formula Aa may be deuterium, at least six of R¹⁹ to R²⁶ of Chemical Formula Aa may be deuterium, and each of R¹⁹ to R²⁶ of Chemical Formula Aa may be deuterium.

For example, Chemical Formula A may be represented by Chemical Formula A-1, below.

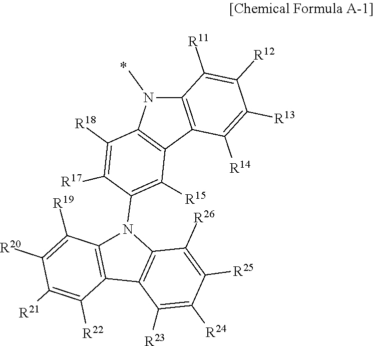

In Chemical Formula A-1, R¹¹ to R¹⁵ and R¹⁷ to R²⁶ may be each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, R¹¹ to R¹⁵ and R¹⁷ to R²⁶ may be each independently present, or two adjacent ones among R¹¹ to R¹⁵ and R¹⁷ to R²⁶ may be linked to form a ring, and * is a linking point with Chemical Formula 1.

For example, the compound may be substituted with at least one deuterium atom, e.g., at least one of R¹ to R¹⁸ of Chemical Formula 1 or Chemical Formula A may be deuterium, and e.g., at least one of R¹ to R¹⁸ and R¹⁹ to R²⁶ of Chemical Formula 1, Chemical Formula A, or Chemical Formula Aa may be deuterium.

For example, R¹⁹ to R²² may each be deuterium.

For example, R¹⁹ to R²⁶ may each be deuterium.

For example, each of R¹¹ to R¹⁸ may be deuterium, except for the group represented by Chemical Formula Aa.

For example, each of R¹¹ to R²⁶ may be deuterium, except for the group represented by Chemical Formula Aa.

For example, each of R¹ to R¹⁰, except for the group represented by Chemical

Formula A, may be deuterium.

For example, each substituent in Chemical Formula 1 may be deuterium or substituted with deuterium.

For example, the compound may be selected from compounds listed in Group 1, below.

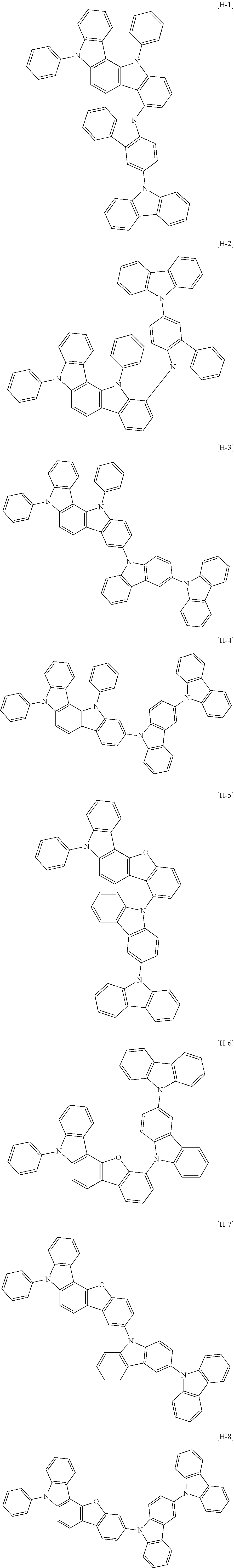

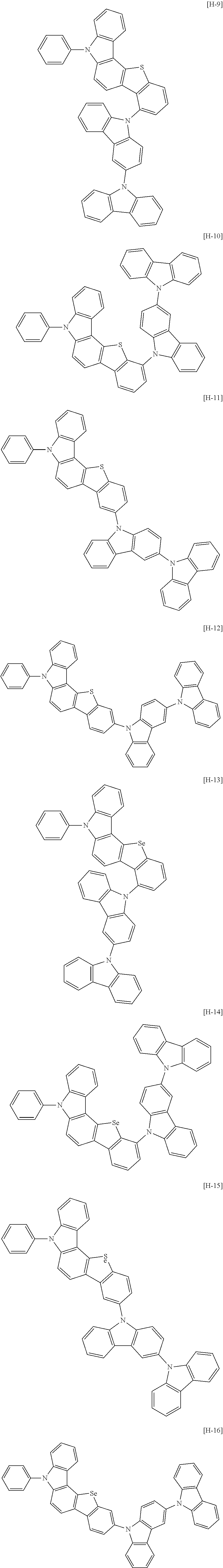

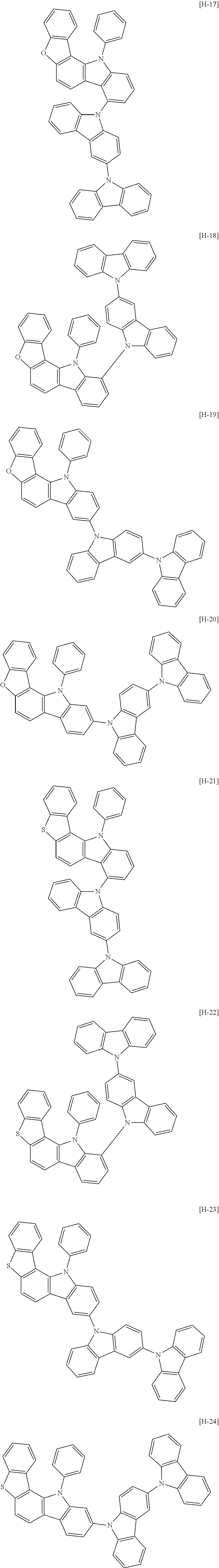

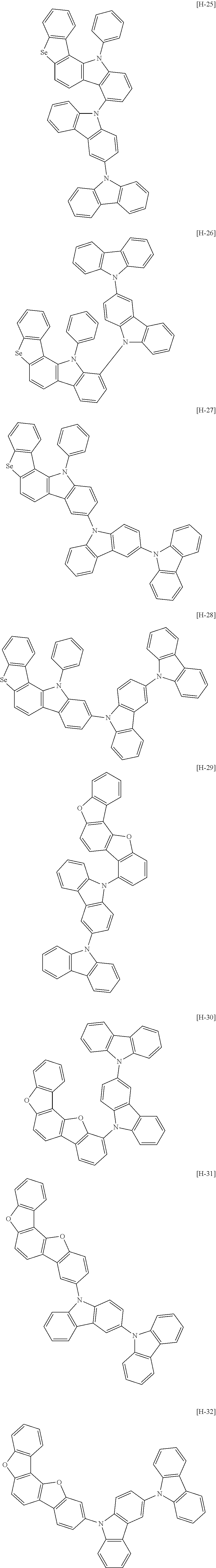

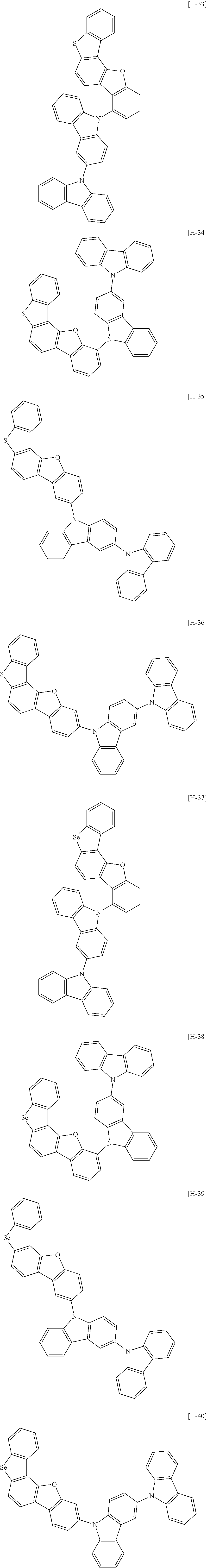

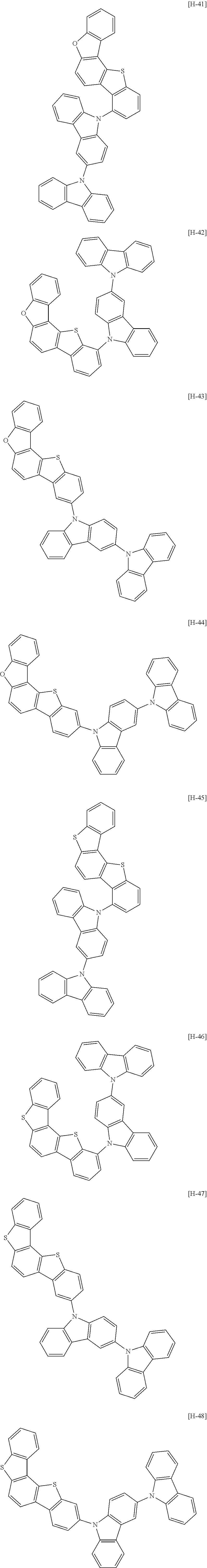

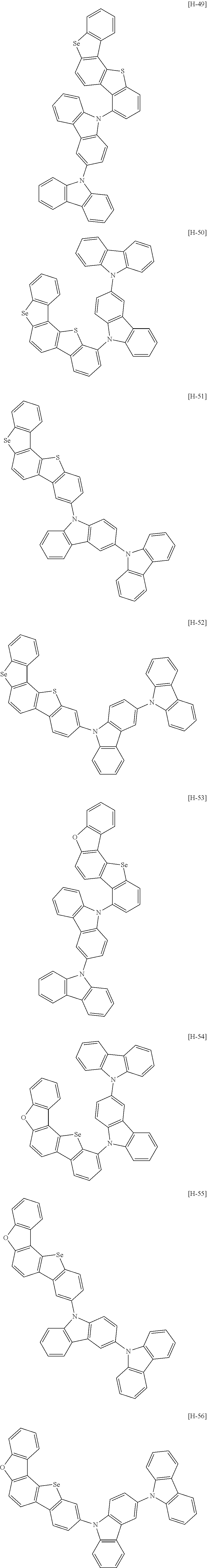

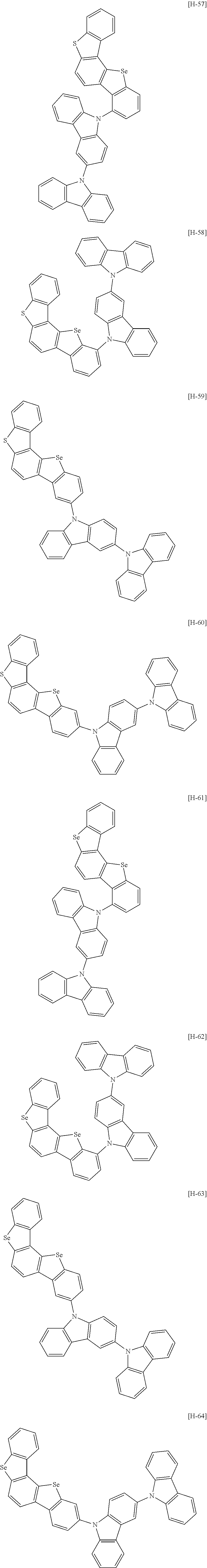

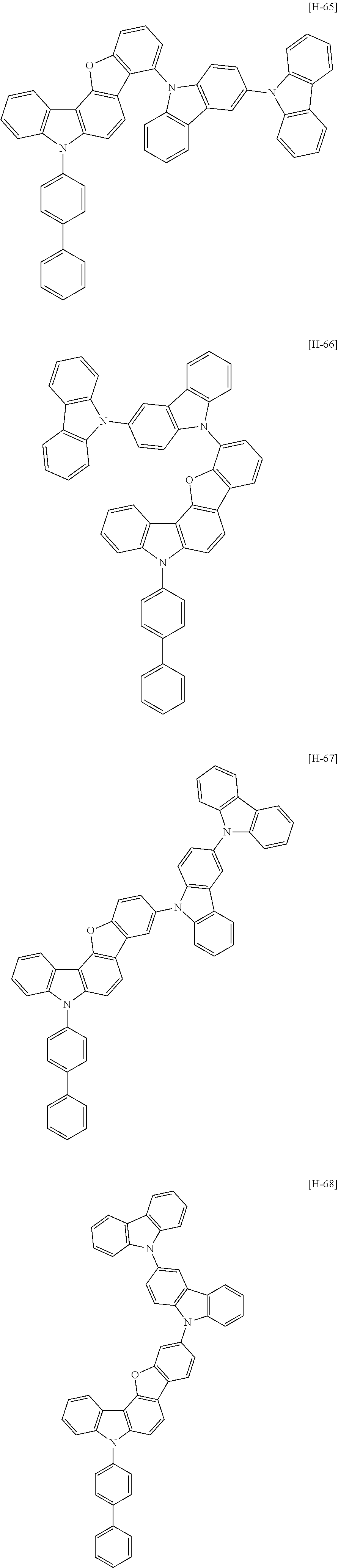 [SMILES: c1ccc(-c2ccc(-n3c4ccccc4c4c5oc6cccc(-n7c8ccccc8c8cc(-n9c%10ccccc%10c%10ccccc%109)ccc87)c6c5ccc43)cc2)cc1 ; c1ccc(-c2ccc(-n3c4ccccc4c4c5oc6c(-n7c8ccccc8c8cc(-n9c%10ccccc%10c%10ccccc%109)ccc87)cccc6c5ccc43)cc2)cc1 ; c1ccc(-c2ccc(-n3c4ccccc4c4c5oc6ccc(-n7c8ccccc8c8cc(-n9c%10ccccc%10c%10ccccc%109)ccc87)cc6c5ccc43)cc2)cc1 ; c1ccc(-c2ccc(-n3c4ccccc4c4c5oc6cc(-n7c8ccccc8c8cc(-n9c%10ccccc%10c%10ccccc%109)ccc87)ccc6c5ccc43)cc2)cc1]

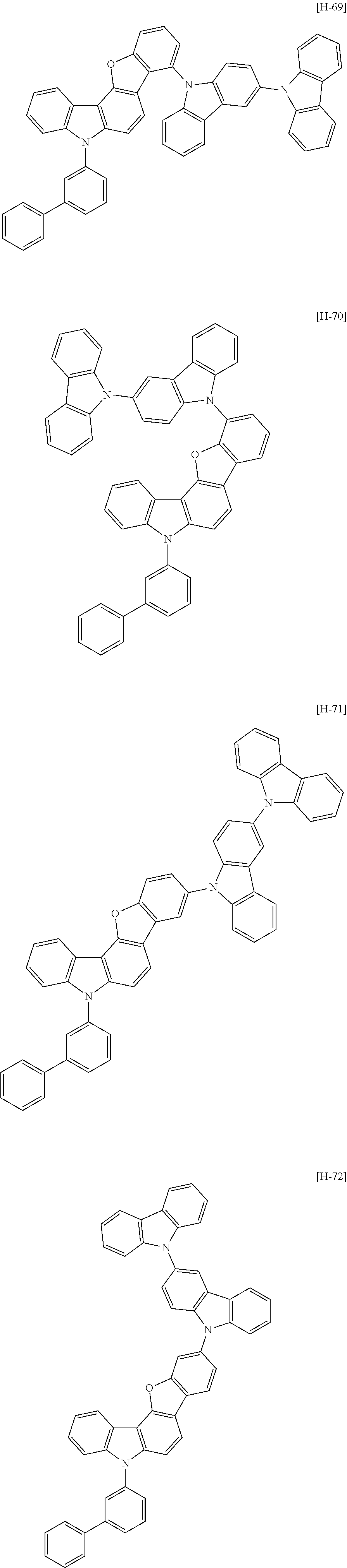 [SMILES: c1ccc(-c2cccc(-n3c4ccccc4c4c5oc6cccc(-n7c8ccccc8c8cc(-n9c%10ccccc%10c%10ccccc%109)ccc87)c6c5ccc43)c2)cc1 ; c1ccc(-c2cccc(-n3c4ccccc4c4c5oc6c(-n7c8ccccc8c8cc(-n9c%10ccccc%10c%10ccccc%109)ccc87)cccc6c5ccc43)c2)cc1 ; c1ccc(-c2cccc(-n3c4ccccc4c4c5oc6ccc(-n7c8ccccc8c8cc(-n9c%10ccccc%10c%10ccccc%109)ccc87)cc6c5ccc43)c2)cc1 ; c1ccc(-c2cccc(-n3c4ccccc4c4c5oc6cc(-n7c8ccccc8c8cc(-n9c%10ccccc%10c%10ccccc%109)ccc87)ccc6c5ccc43)c2)cc1]

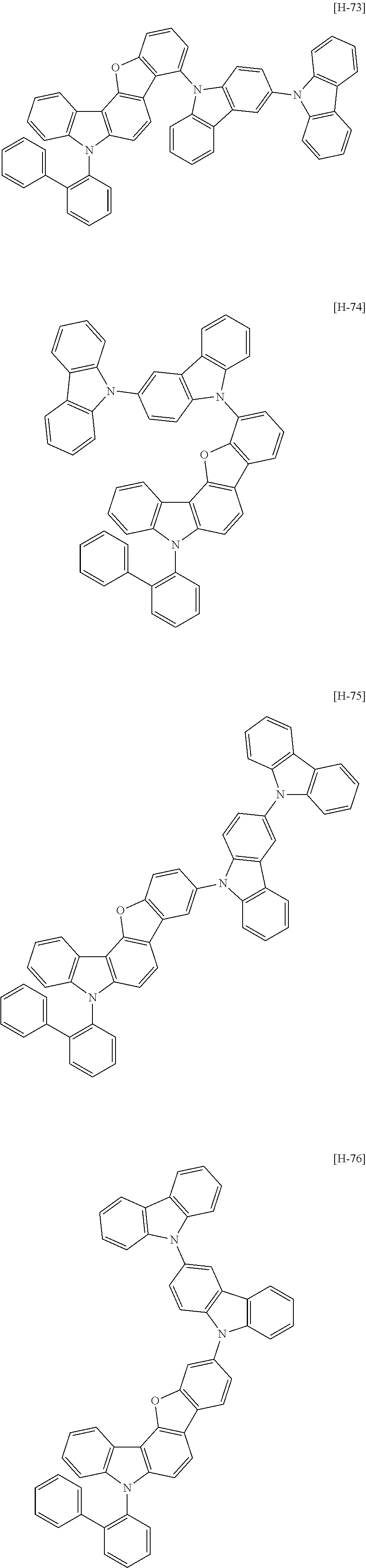 [SMILES: c1ccc(-c2ccccc2-n2c3ccccc3c3c4oc5cccc(-n6c7ccccc7c7cc(-n8c9ccccc9c9ccccc98)ccc76)c5c4ccc32)cc1 ; c1ccc(-c2ccccc2-n2c3ccccc3c3c4oc5c(-n6c7ccccc7c7cc(-n8c9ccccc9c9ccccc98)ccc76)cccc5c4ccc32)cc1 ; c1ccc(-c2ccccc2-n2c3ccccc3c3c4oc5ccc(-n6c7ccccc7c7cc(-n8c9ccccc9c9ccccc98)ccc76)cc5c4ccc32)cc1 ; c1ccc(-c2ccccc2-n2c3ccccc3c3c4oc5cc(-n6c7ccccc7c7cc(-n8c9ccccc9c9ccccc98)ccc76)ccc5c4ccc32)cc1]

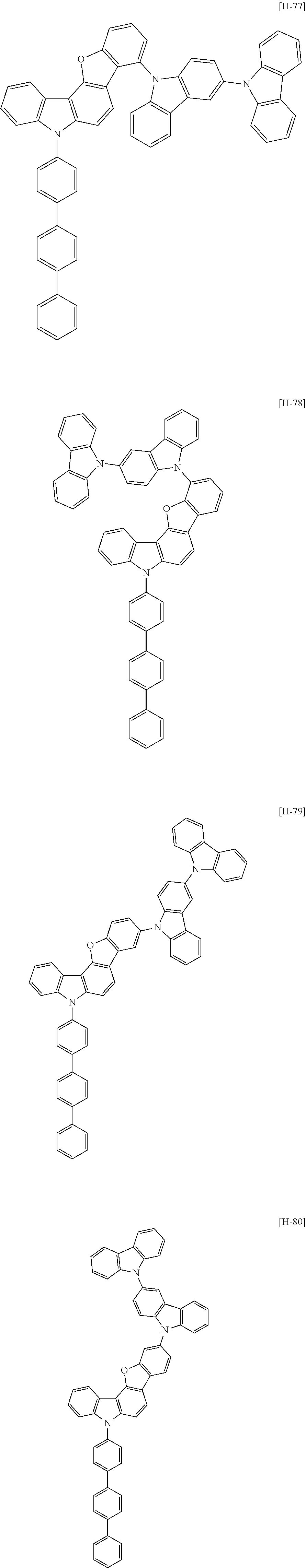 [SMILES: c1ccc(-c2ccc(-c3ccc(-n4c5ccccc5c5c6oc7cccc(-n8c9ccccc9c9cc(-n%10c%11ccccc%11c%11ccccc%11%10)ccc98)c7c6ccc54)cc3)cc2)cc1 ; c1ccc(-c2ccc(-c3ccc(-n4c5ccccc5c5c6oc7c(-n8c9ccccc9c9cc(-n%10c%11ccccc%11c%11ccccc%11%10)ccc98)cccc7c6ccc54)cc3)cc2)cc1 ; c1ccc(-c2ccc(-c3ccc(-n4c5ccccc5c5c6oc7ccc(-n8c9ccccc9c9cc(-n%10c%11ccccc%11c%11ccccc%11%10)ccc98)cc7c6ccc54)cc3)cc2)cc1 ; c1ccc(-c2ccc(-c3ccc(-n4c5ccccc5c5c6oc7cc(-n8c9ccccc9c9cc(-n%10c%11ccccc%11c%11ccccc%11%10)ccc98)ccc7c6ccc54)cc3)cc2)cc1]

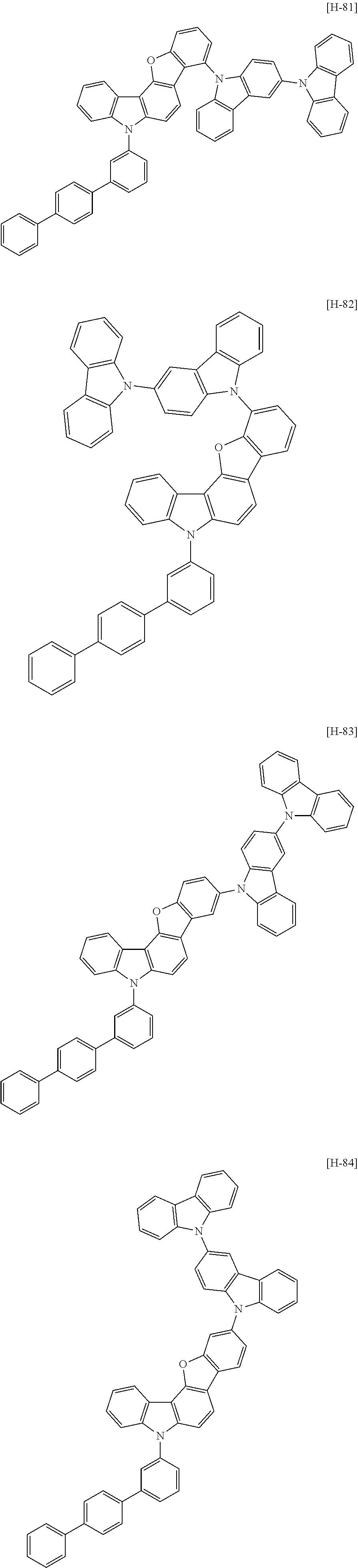 [SMILES: c1ccc(-c2ccc(-c3cccc(-n4c5ccccc5c5c6oc7cccc(-n8c9ccccc9c9cc(-n%10c%11ccccc%11c%11ccccc%11%10)ccc98)c7c6ccc54)c3)cc2)cc1 ; c1ccc(-c2ccc(-c3cccc(-n4c5ccccc5c5c6oc7c(-n8c9ccccc9c9cc(-n%10c%11ccccc%11c%11ccccc%11%10)ccc98)cccc7c6ccc54)c3)cc2)cc1 ; c1ccc(-c2ccc(-c3cccc(-n4c5ccccc5c5c6oc7ccc(-n8c9ccccc9c9cc(-n%10c%11ccccc%11c%11ccccc%11%10)ccc98)cc7c6ccc54)c3)cc2)cc1 ; c1ccc(-c2ccc(-c3cccc(-n4c5ccccc5c5c6oc7cc(-n8c9ccccc9c9cc(-n%10c%11ccccc%11c%11ccccc%11%10)ccc98)ccc7c6ccc54)c3)cc2)cc1]

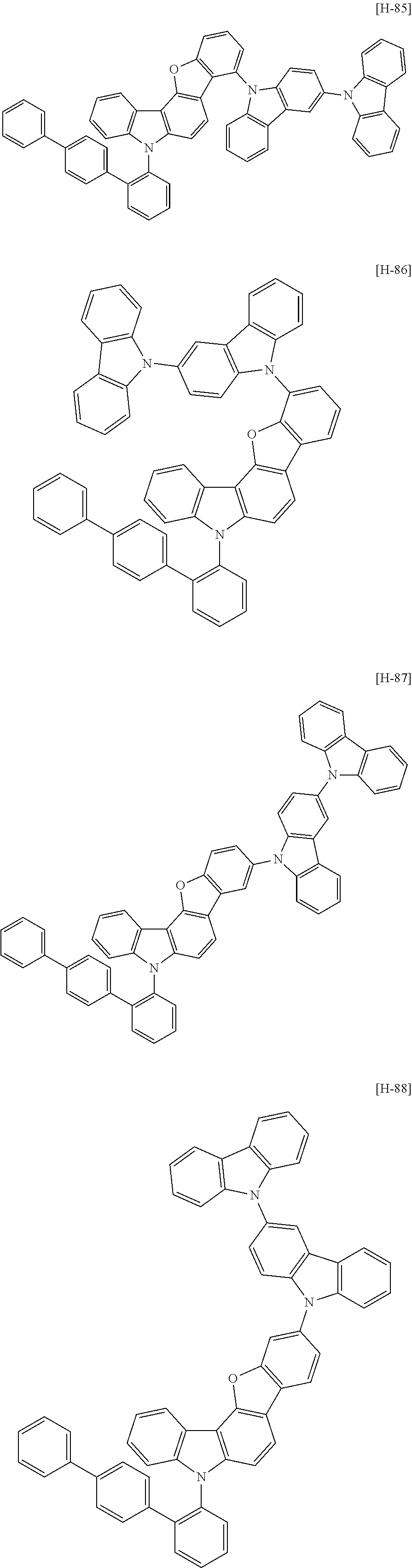 [SMILES: c1ccc(-c2ccc(-c3ccccc3-n3c4ccccc4c4c5oc6cccc(-n7c8ccccc8c8cc(-n9c%10ccccc%10c%10ccccc%109)ccc87)c6c5ccc43)cc2)cc1 ; c1ccc(-c2ccc(-c3ccccc3-n3c4ccccc4c4c5oc6c(-n7c8ccccc8c8cc(-n9c%10ccccc%10c%10ccccc%109)ccc87)cccc6c5ccc43)cc2)cc1 ; c1ccc(-c2ccc(-c3ccccc3-n3c4ccccc4c4c5oc6ccc(-n7c8ccccc8c8cc(-n9c%10ccccc%10c%10ccccc%109)ccc87)cc6c5ccc43)cc2)cc1 ; c1ccc(-c2ccc(-c3ccccc3-n3c4ccccc4c4c5oc6cc(-n7c8ccccc8c8cc(-n9c%10ccccc%10c%10ccccc%109)ccc87)ccc6c5ccc43)cc2)cc1]

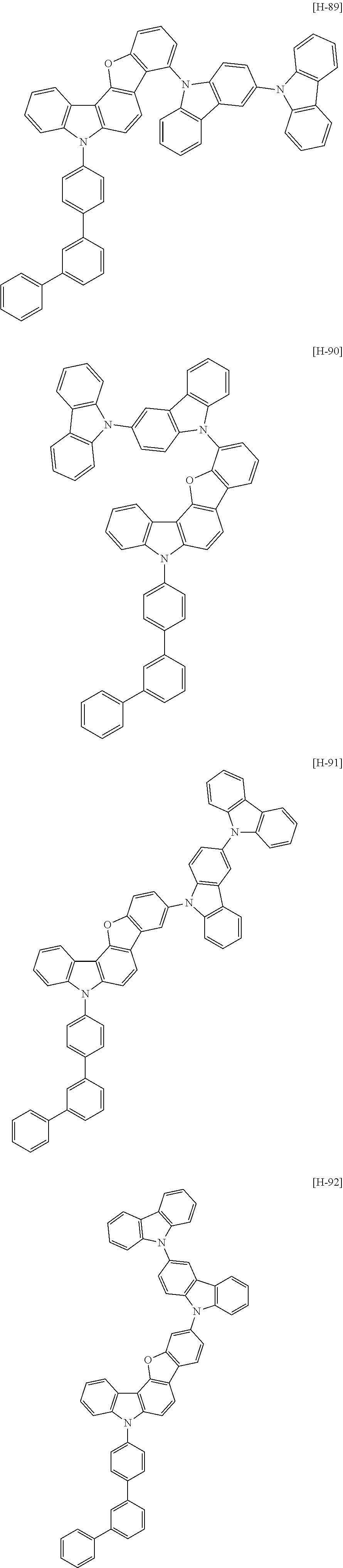 [SMILES: c1ccc(-c2cccc(-c3ccc(-n4c5ccccc5c5c6oc7cccc(-n8c9ccccc9c9cc(-n%10c%11ccccc%11c%11ccccc%11%10)ccc98)c7c6ccc54)cc3)c2)cc1 ; c1ccc(-c2cccc(-c3ccc(-n4c5ccccc5c5c6oc7c(-n8c9ccccc9c9cc(-n%10c%11ccccc%11c%11ccccc%11%10)ccc98)cccc7c6ccc54)cc3)c2)cc1 ; c1ccc(-c2cccc(-c3ccc(-n4c5ccccc5c5c6oc7ccc(-n8c9ccccc9c9cc(-n%10c%11ccccc%11c%11ccccc%11%10)ccc98)cc7c6ccc54)cc3)c2)cc1 ; c1ccc(-c2cccc(-c3ccc(-n4c5ccccc5c5c6oc7cc(-n8c9ccccc9c9cc(-n%10c%11ccccc%11c%11ccccc%11%10)ccc98)ccc7c6ccc54)cc3)c2)cc1]

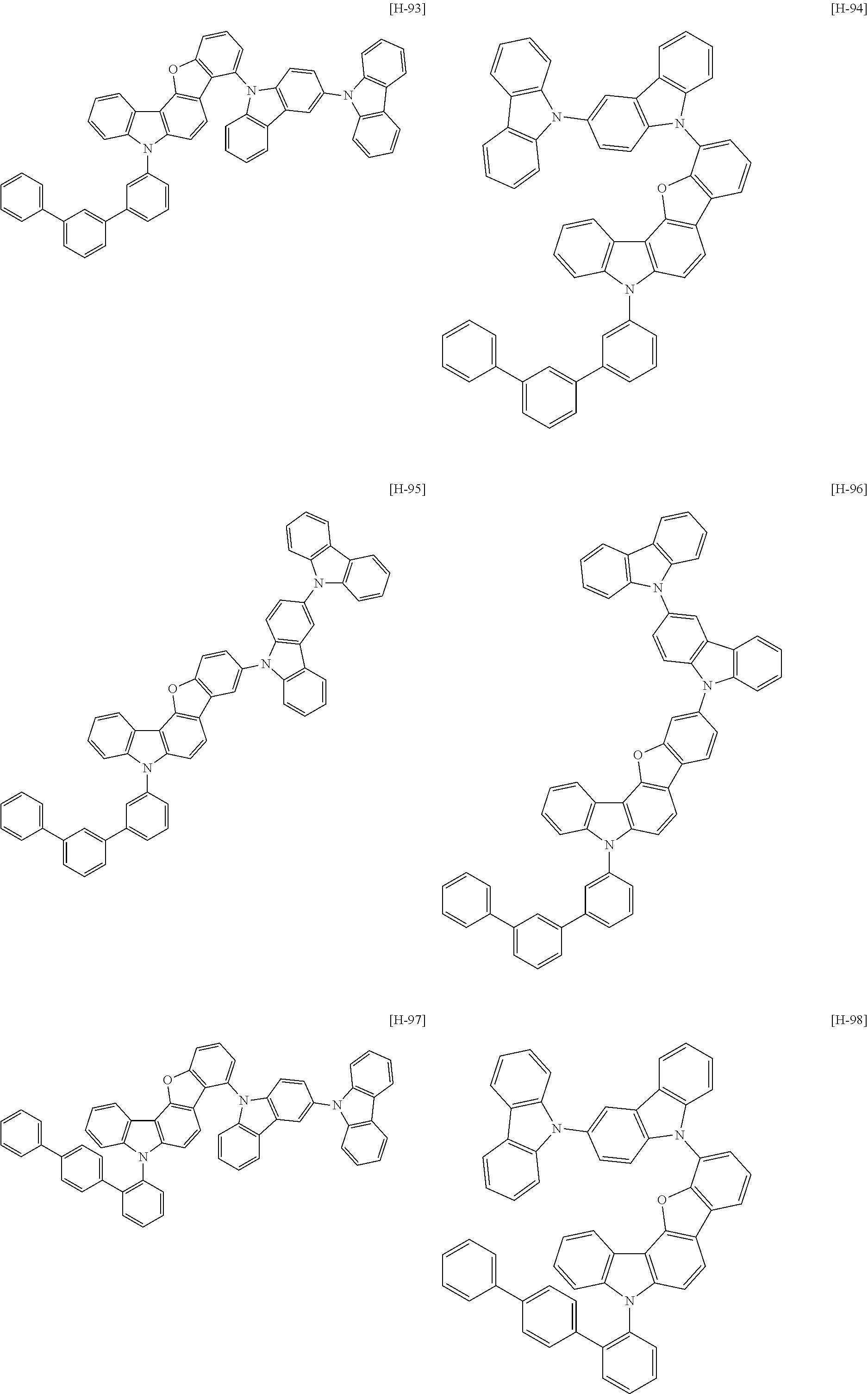

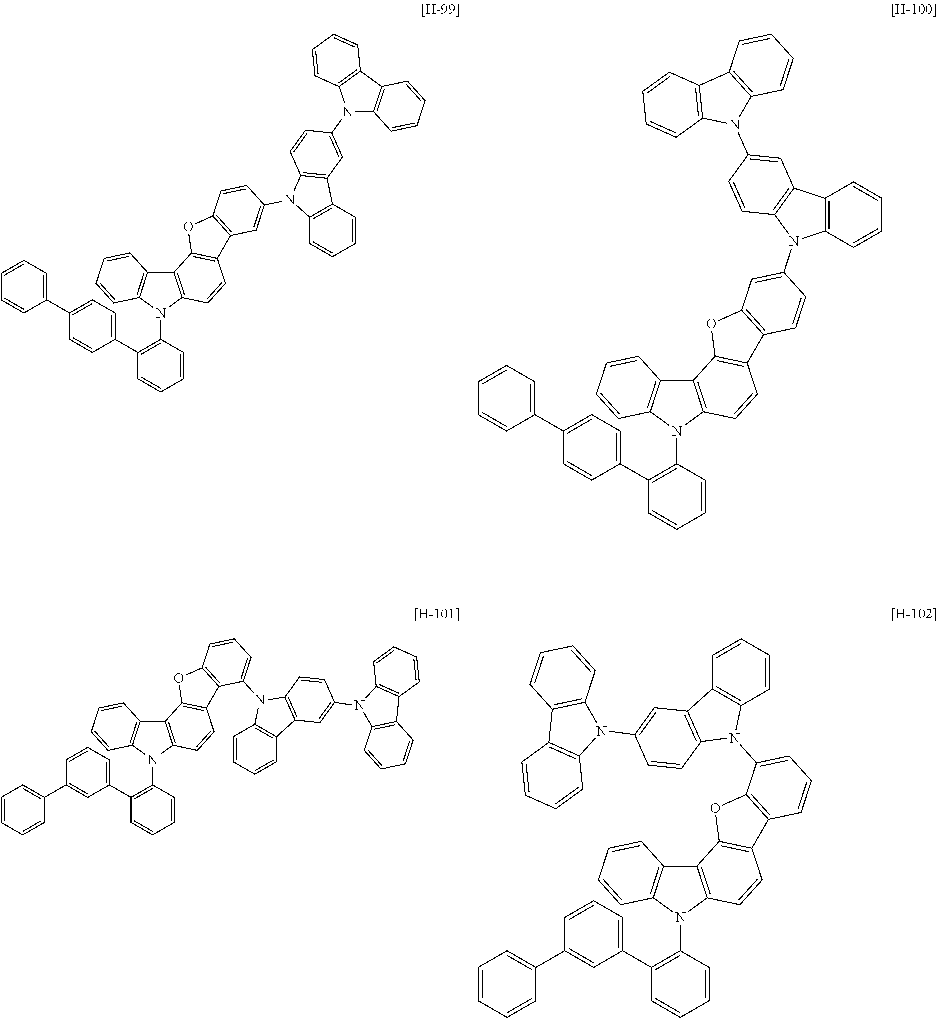 [SMILES: c1ccc(-c2ccc(-c3ccccc3-n3c4ccccc4c4c5oc6ccc(-n7c8ccccc8c8cc(-n9c%10ccccc%10c%10ccccc%109)ccc87)cc6c5ccc43)cc2)cc1 ; c1ccc(-c2ccc(-c3ccccc3-n3c4ccccc4c4c5oc6cc(-n7c8ccccc8c8cc(-n9c%10ccccc%10c%10ccccc%109)ccc87)ccc6c5ccc43)cc2)cc1 ; c1ccc(-c2cccc(-c3ccccc3-n3c4ccccc4c4c5oc6cccc(-n7c8ccccc8c8cc(-n9c%10ccccc%10c%10ccccc%109)ccc87)c6c5ccc43)c2)cc1 ; c1ccc(-c2cccc(-c3ccccc3-n3c4ccccc4c4c5oc6c(-n7c8ccccc8c8cc(-n9c%10ccccc%10c%10ccccc%109)ccc87)cccc6c5ccc43)c2)cc1]

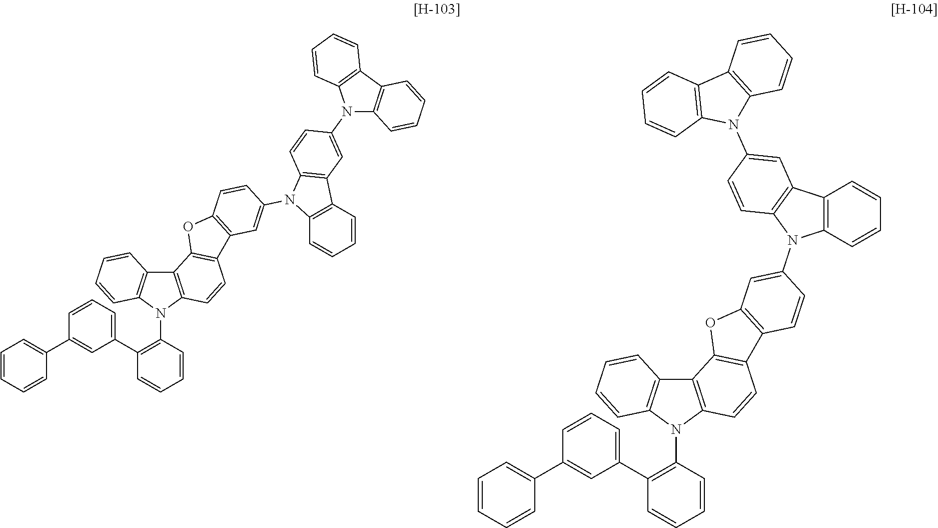 [SMILES: c1ccc(-c2cccc(-c3ccccc3-n3c4ccccc4c4c5oc6ccc(-n7c8ccccc8c8cc(-n9c%10ccccc%10c%10ccccc%109)ccc87)cc6c5ccc43)c2)cc1 ; c1ccc(-c2cccc(-c3ccccc3-n3c4ccccc4c4c5oc6cc(-n7c8ccccc8c8cc(-n9c%10ccccc%10c%10ccccc%109)ccc87)ccc6c5ccc43)c2)cc1]

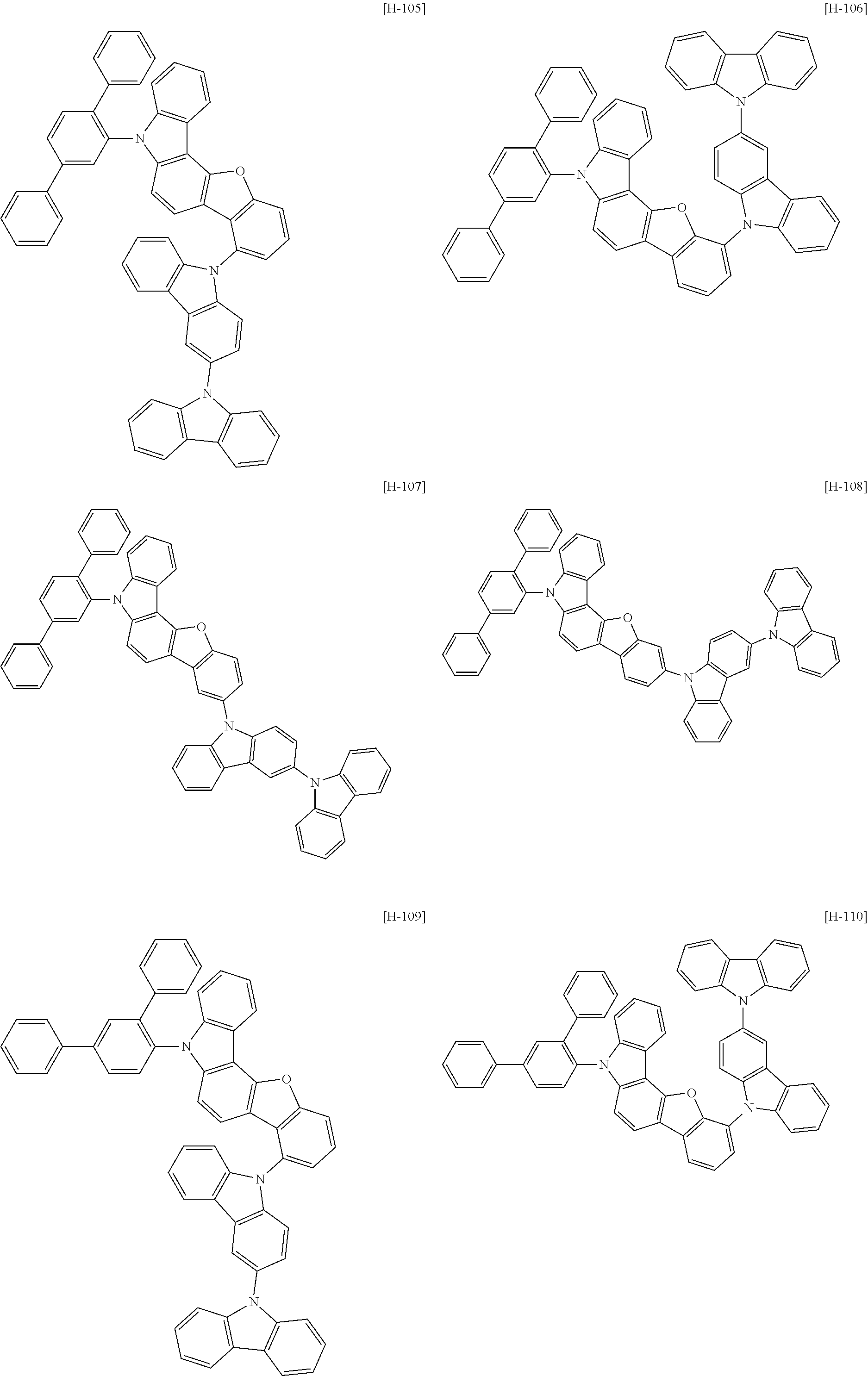

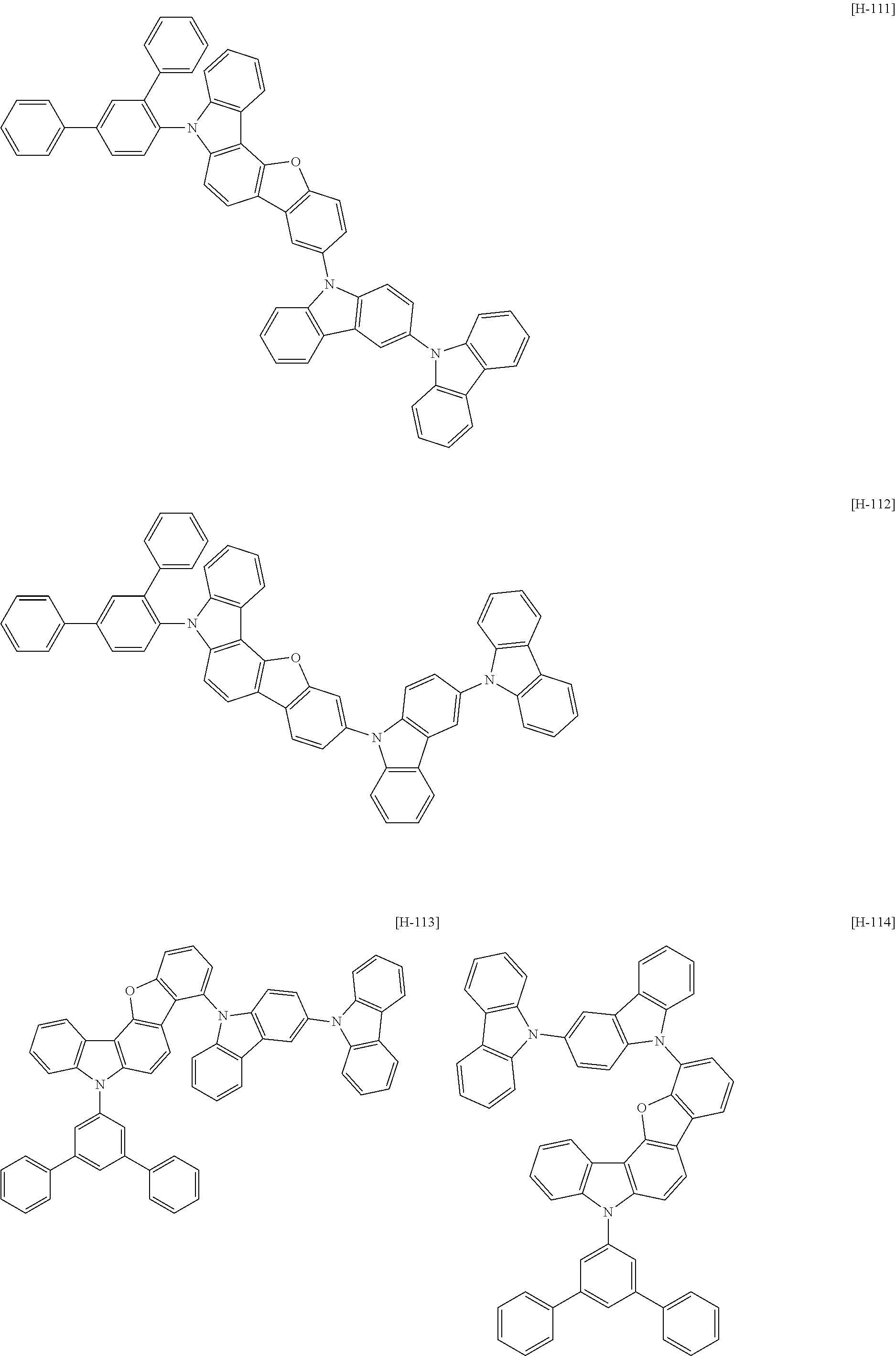 [SMILES: c1ccc(-c2ccc(-n3c4ccccc4c4c5oc6ccc(-n7c8ccccc8c8cc(-n9c%10ccccc%10c%10ccccc%109)ccc87)cc6c5ccc43)c(-c3ccccc3)c2)cc1 ; c1ccc(-c2ccc(-n3c4ccccc4c4c5oc6cc(-n7c8ccccc8c8cc(-n9c%10ccccc%10c%10ccccc%109)ccc87)ccc6c5ccc43)c(-c3ccccc3)c2)cc1 ; c1ccc(-c2cc(-c3ccccc3)cc(-n3c4ccccc4c4c5oc6cccc(-n7c8ccccc8c8cc(-n9c%10ccccc%10c%10ccccc%109)ccc87)c6c5ccc43)c2)cc1 ; c1ccc(-c2cc(-c3ccccc3)cc(-n3c4ccccc4c4c5oc6c(-n7c8ccccc8c8cc(-n9c%10ccccc%10c%10ccccc%109)ccc87)cccc6c5ccc43)c2)cc1]

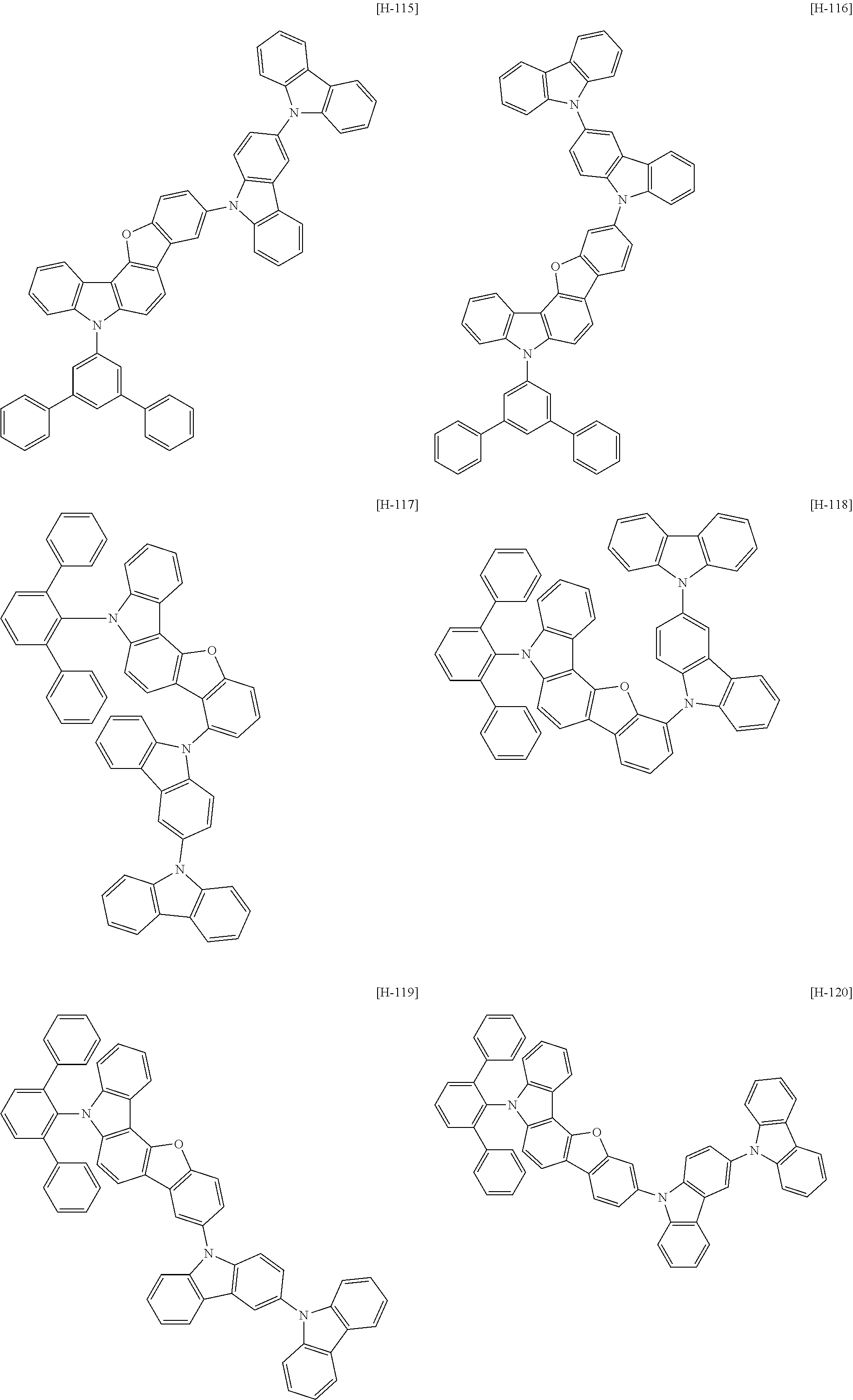

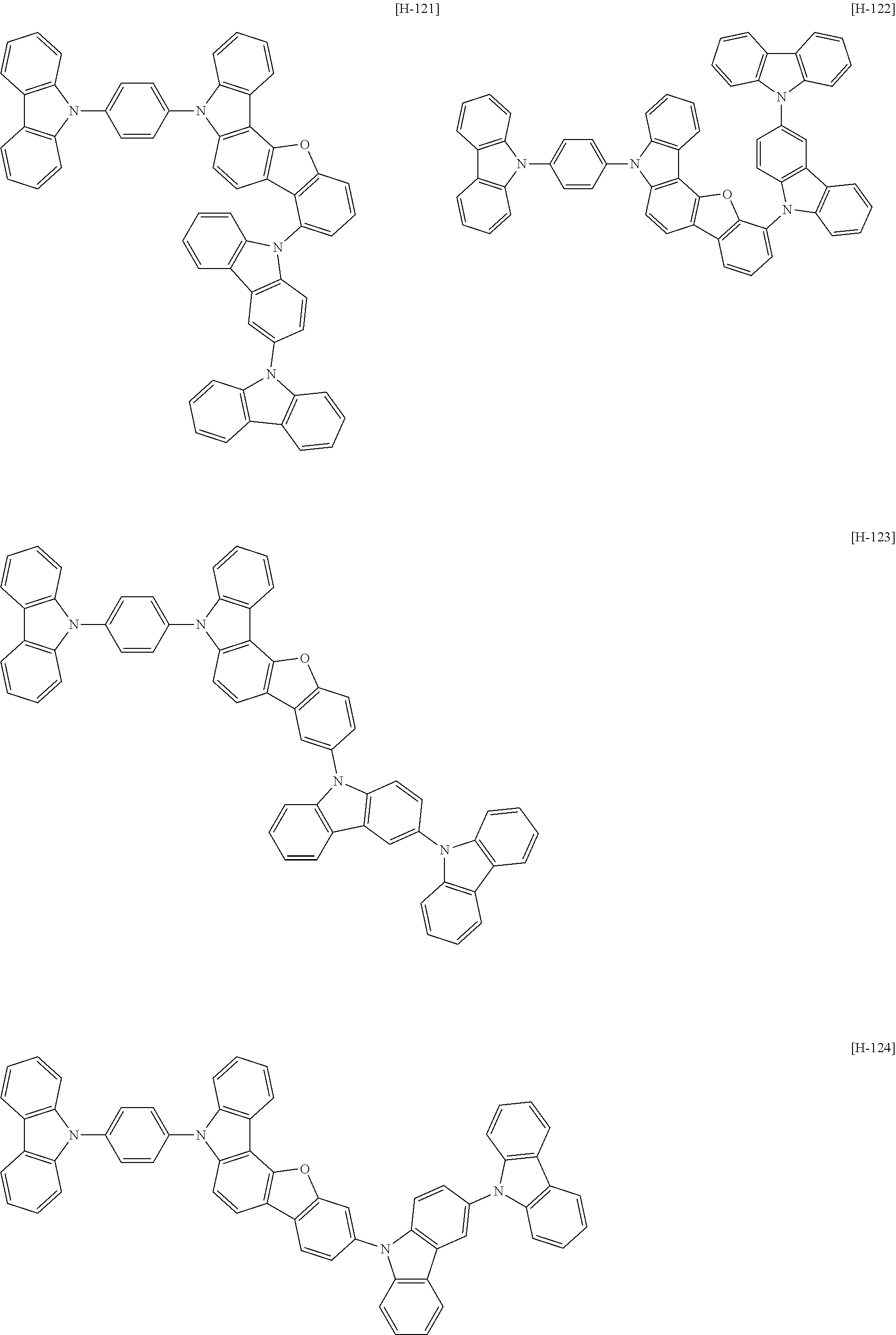 [SMILES: c1cc(-n2c3ccccc3c3cc(-n4c5ccccc5c5ccccc54)ccc32)c2c(c1)oc1c2ccc2c1c1ccccc1n2-c1ccc(-n2c3ccccc3c3ccccc32)cc1 ; c1cc(-n2c3ccccc3c3cc(-n4c5ccccc5c5ccccc54)ccc32)c2oc3c(ccc4c3c3ccccc3n4-c3ccc(-n4c5ccccc5c5ccccc54)cc3)c2c1 ; c1ccc2c(c1)c1ccccc1n2-c1ccc(-n2c3ccccc3c3c4oc5ccc(-n6c7ccccc7c7cc(-n8c9ccccc9c9ccccc98)ccc76)cc5c4ccc32)cc1 ; c1ccc2c(c1)c1ccccc1n2-c1ccc(-n2c3ccccc3c3c4oc5cc(-n6c7ccccc7c7cc(-n8c9ccccc9c9ccccc98)ccc76)ccc5c4ccc32)cc1]

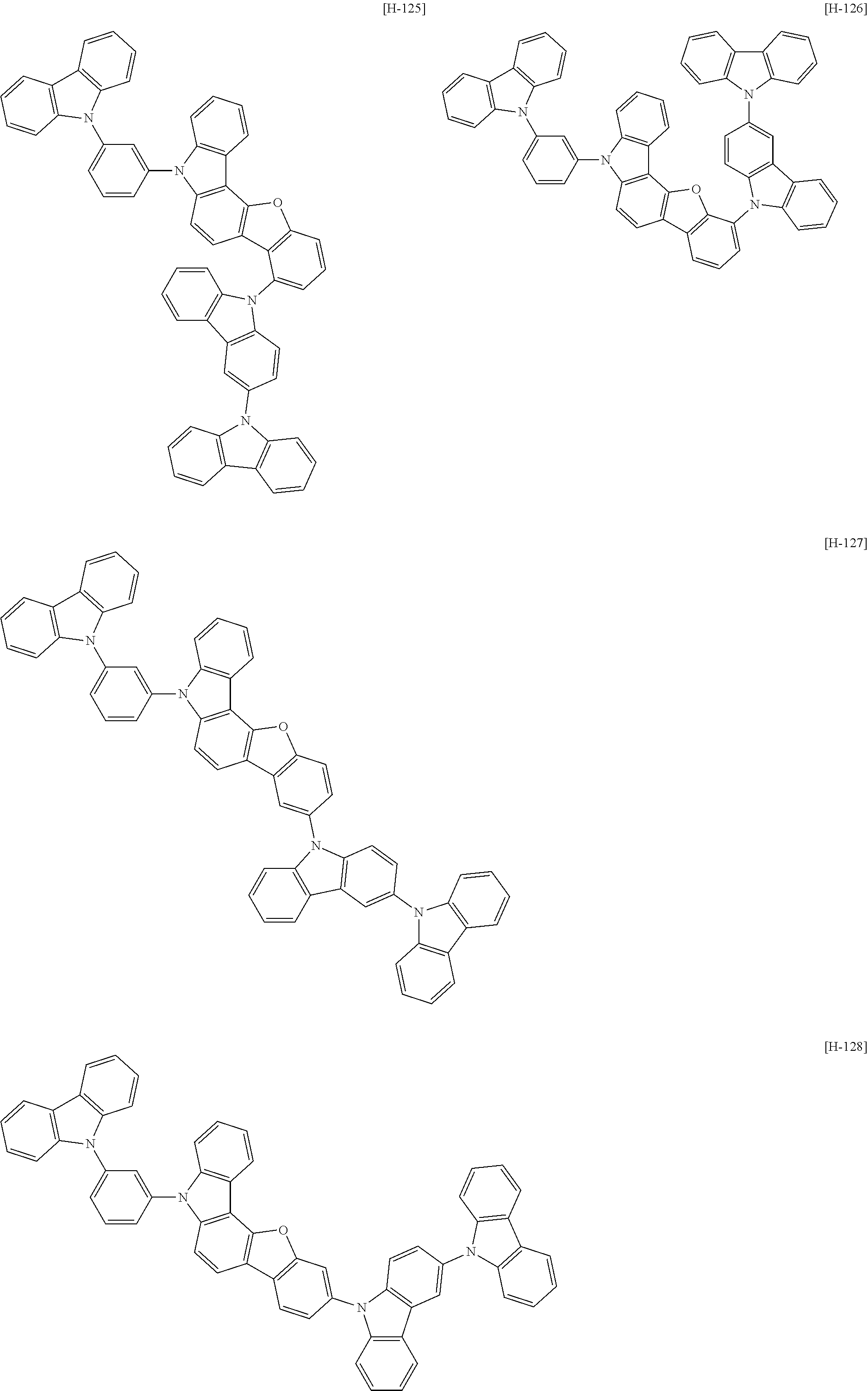 [SMILES: c1cc(-n2c3ccccc3c3ccccc32)cc(-n2c3ccccc3c3c4oc5cccc(-n6c7ccccc7c7cc(-n8c9ccccc9c9ccccc98)ccc76)c5c4ccc32)c1 ; c1cc(-n2c3ccccc3c3ccccc32)cc(-n2c3ccccc3c3c4oc5c(-n6c7ccccc7c7cc(-n8c9ccccc9c9ccccc98)ccc76)cccc5c4ccc32)c1 ; c1cc(-n2c3ccccc3c3ccccc32)cc(-n2c3ccccc3c3c4oc5ccc(-n6c7ccccc7c7cc(-n8c9ccccc9c9ccccc98)ccc76)cc5c4ccc32)c1 ; c1cc(-n2c3ccccc3c3ccccc32)cc(-n2c3ccccc3c3c4oc5cc(-n6c7ccccc7c7cc(-n8c9ccccc9c9ccccc98)ccc76)ccc5c4ccc32)c1]

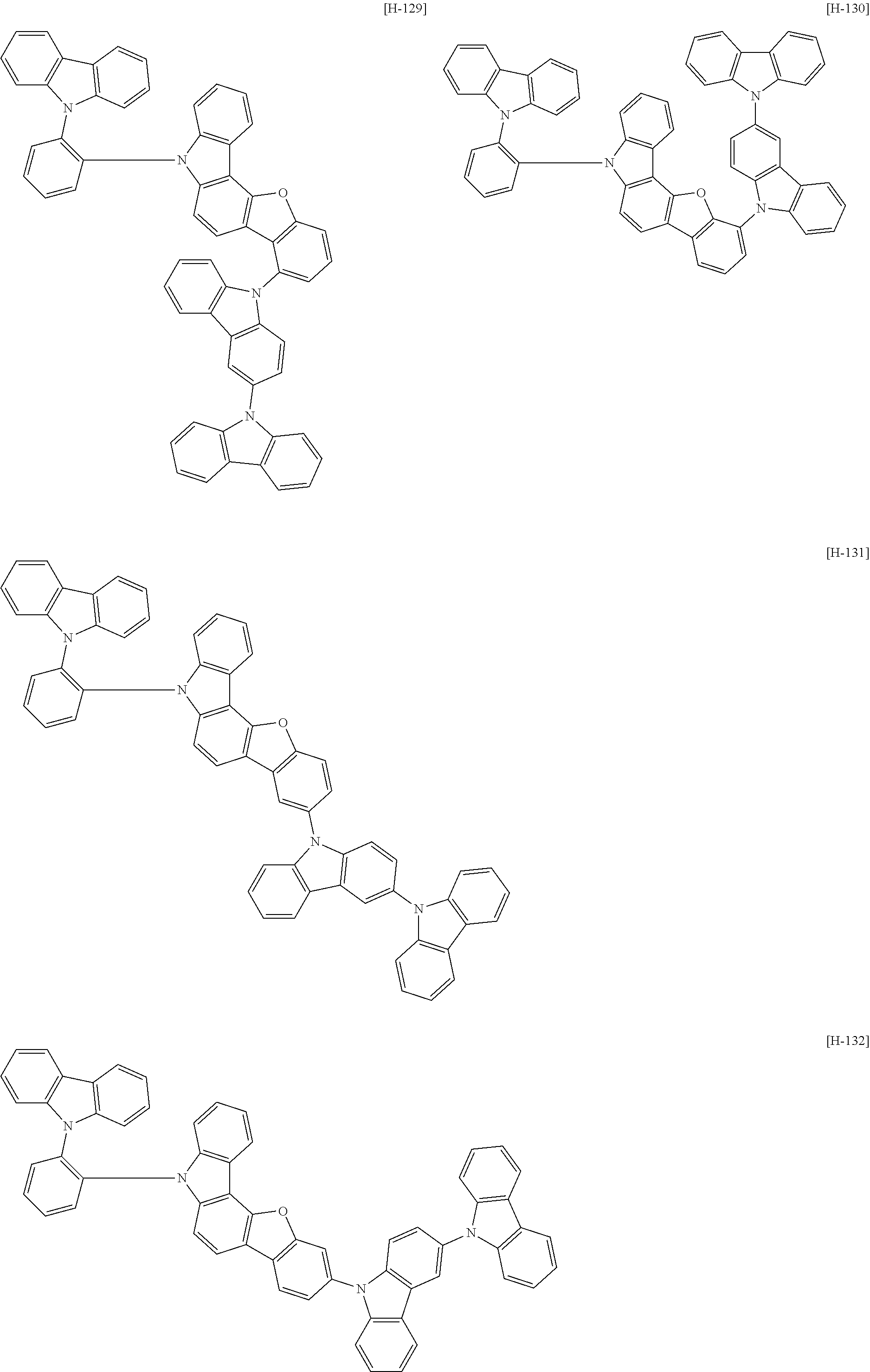 [SMILES: c1ccc(-n2c3ccccc3c3c4oc5cccc(-n6c7ccccc7c7cc(-n8c9ccccc9c9ccccc98)ccc76)c5c4ccc32)c(-n2c3ccccc3c3ccccc32)c1 ; c1ccc(-n2c3ccccc3c3c4oc5c(-n6c7ccccc7c7cc(-n8c9ccccc9c9ccccc98)ccc76)cccc5c4ccc32)c(-n2c3ccccc3c3ccccc32)c1 ; c1ccc(-n2c3ccccc3c3c4oc5ccc(-n6c7ccccc7c7cc(-n8c9ccccc9c9ccccc98)ccc76)cc5c4ccc32)c(-n2c3ccccc3c3ccccc32)c1 ; c1ccc(-n2c3ccccc3c3c4oc5cc(-n6c7ccccc7c7cc(-n8c9ccccc9c9ccccc98)ccc76)ccc5c4ccc32)c(-n2c3ccccc3c3ccccc32)c1]

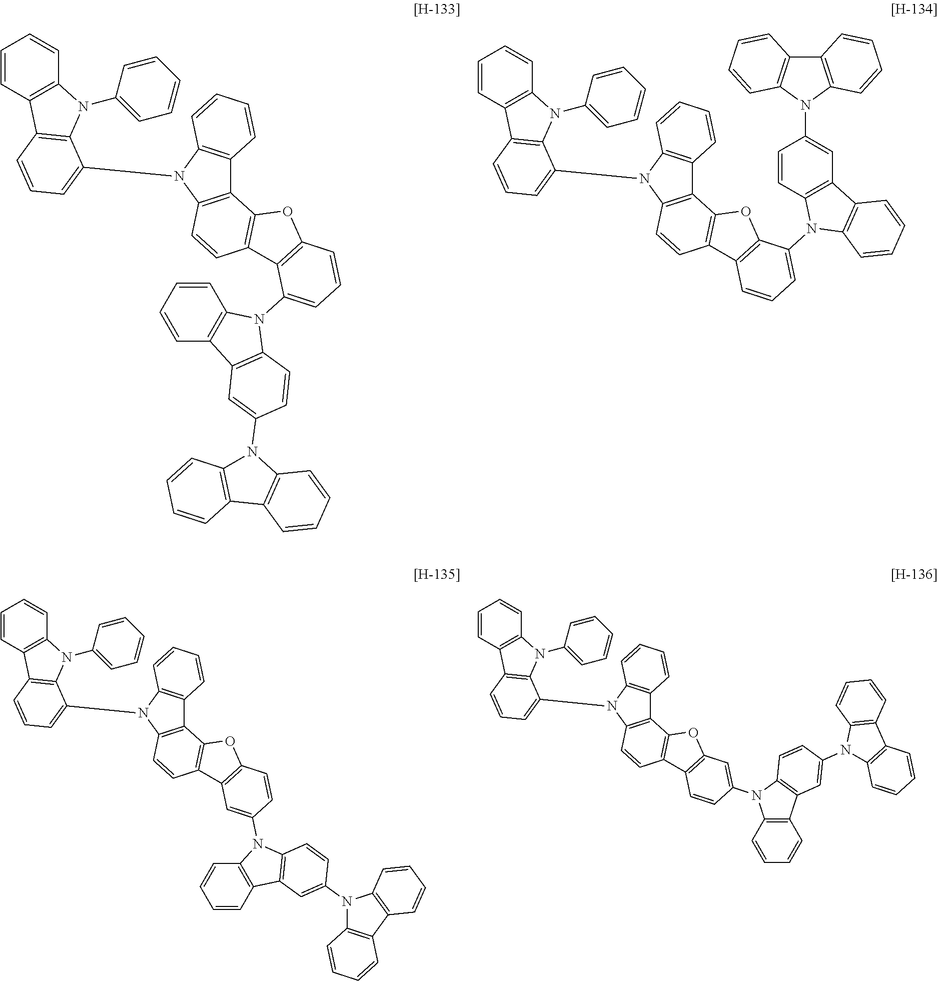 [SMILES: c1ccc(-n2c3ccccc3c3cccc(-n4c5ccccc5c5c6oc7cccc(-n8c9ccccc9c9cc(-n%10c%11ccccc%11c%11ccccc%11%10)ccc98)c7c6ccc54)c32)cc1 ; c1ccc(-n2c3ccccc3c3cccc(-n4c5ccccc5c5c6oc7c(-n8c9ccccc9c9cc(-n%10c%11ccccc%11c%11ccccc%11%10)ccc98)cccc7c6ccc54)c32)cc1 ; c1ccc(-n2c3ccccc3c3cccc(-n4c5ccccc5c5c6oc7ccc(-n8c9ccccc9c9cc(-n%10c%11ccccc%11c%11ccccc%11%10)ccc98)cc7c6ccc54)c32)cc1 ; c1ccc(-n2c3ccccc3c3cccc(-n4c5ccccc5c5c6oc7cc(-n8c9ccccc9c9cc(-n%10c%11ccccc%11c%11ccccc%11%10)ccc98)ccc7c6ccc54)c32)cc1]

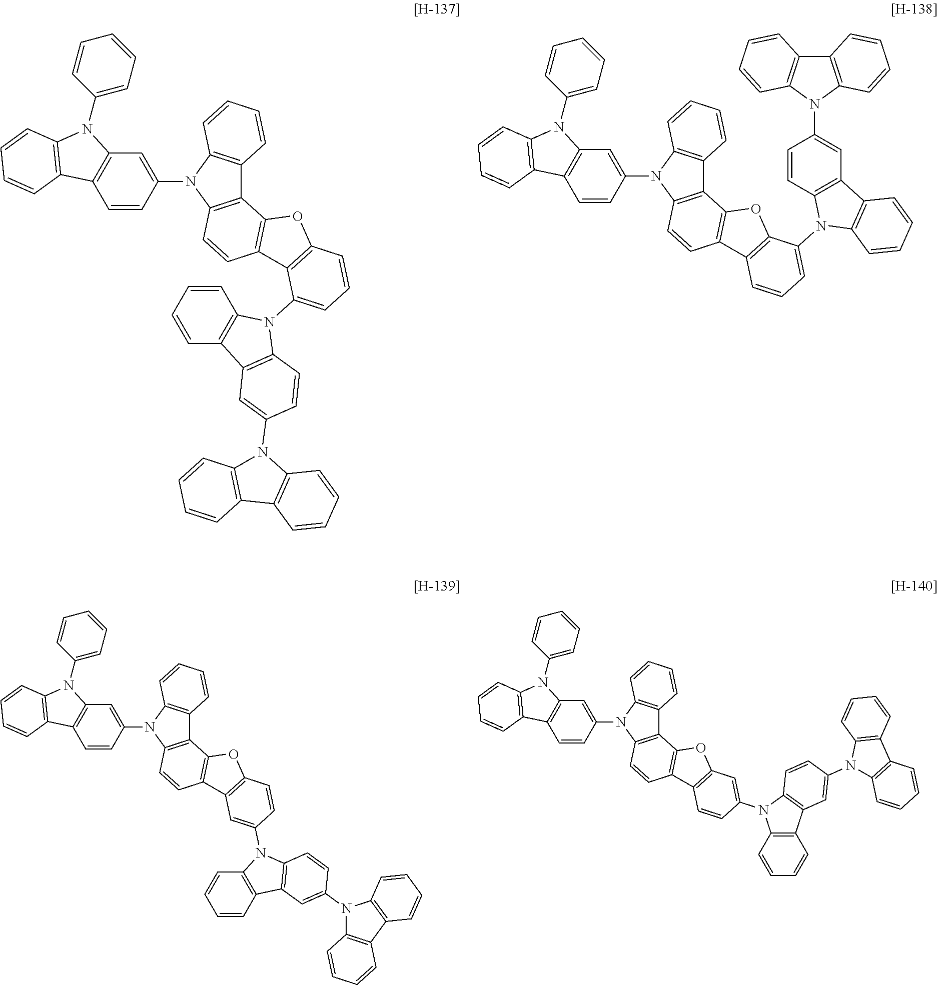 [SMILES: c1ccc(-n2c3ccccc3c3ccc(-n4c5ccccc5c5c6oc7cccc(-n8c9ccccc9c9cc(-n%10c%11ccccc%11c%11ccccc%11%10)ccc98)c7c6ccc54)cc32)cc1 ; c1ccc(-n2c3ccccc3c3ccc(-n4c5ccccc5c5c6oc7c(-n8c9ccccc9c9cc(-n%10c%11ccccc%11c%11ccccc%11%10)ccc98)cccc7c6ccc54)cc32)cc1 ; c1ccc(-n2c3ccccc3c3ccc(-n4c5ccccc5c5c6oc7ccc(-n8c9ccccc9c9cc(-n%10c%11ccccc%11c%11ccccc%11%10)ccc98)cc7c6ccc54)cc32)cc1 ; c1ccc(-n2c3ccccc3c3ccc(-n4c5ccccc5c5c6oc7cc(-n8c9ccccc9c9cc(-n%10c%11ccccc%11c%11ccccc%11%10)ccc98)ccc7c6ccc54)cc32)cc1]

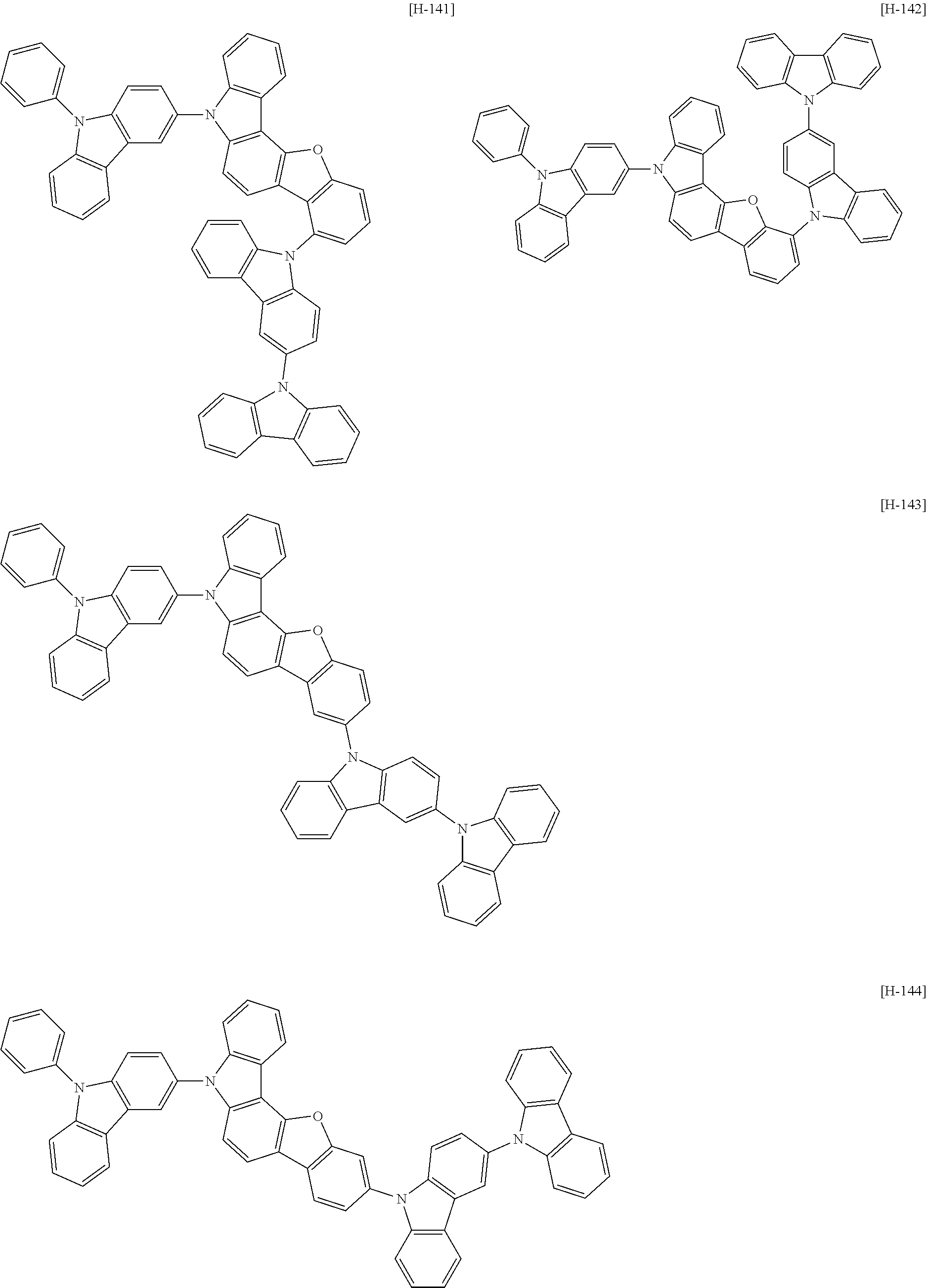 [SMILES: c1ccc(-n2c3ccccc3c3cc(-n4c5ccccc5c5c6oc7cccc(-n8c9ccccc9c9cc(-n%10c%11ccccc%11c%11ccccc%11%10)ccc98)c7c6ccc54)ccc32)cc1 ; c1ccc(-n2c3ccccc3c3cc(-n4c5ccccc5c5c6oc7c(-n8c9ccccc9c9cc(-n%10c%11ccccc%11c%11ccccc%11%10)ccc98)cccc7c6ccc54)ccc32)cc1 ; c1ccc(-n2c3ccccc3c3cc(-n4c5ccccc5c5c6oc7ccc(-n8c9ccccc9c9cc(-n%10c%11ccccc%11c%11ccccc%11%10)ccc98)cc7c6ccc54)ccc32)cc1 ; c1ccc(-n2c3ccccc3c3cc(-n4c5ccccc5c5c6oc7cc(-n8c9ccccc9c9cc(-n%10c%11ccccc%11c%11ccccc%11%10)ccc98)ccc7c6ccc54)ccc32)cc1]

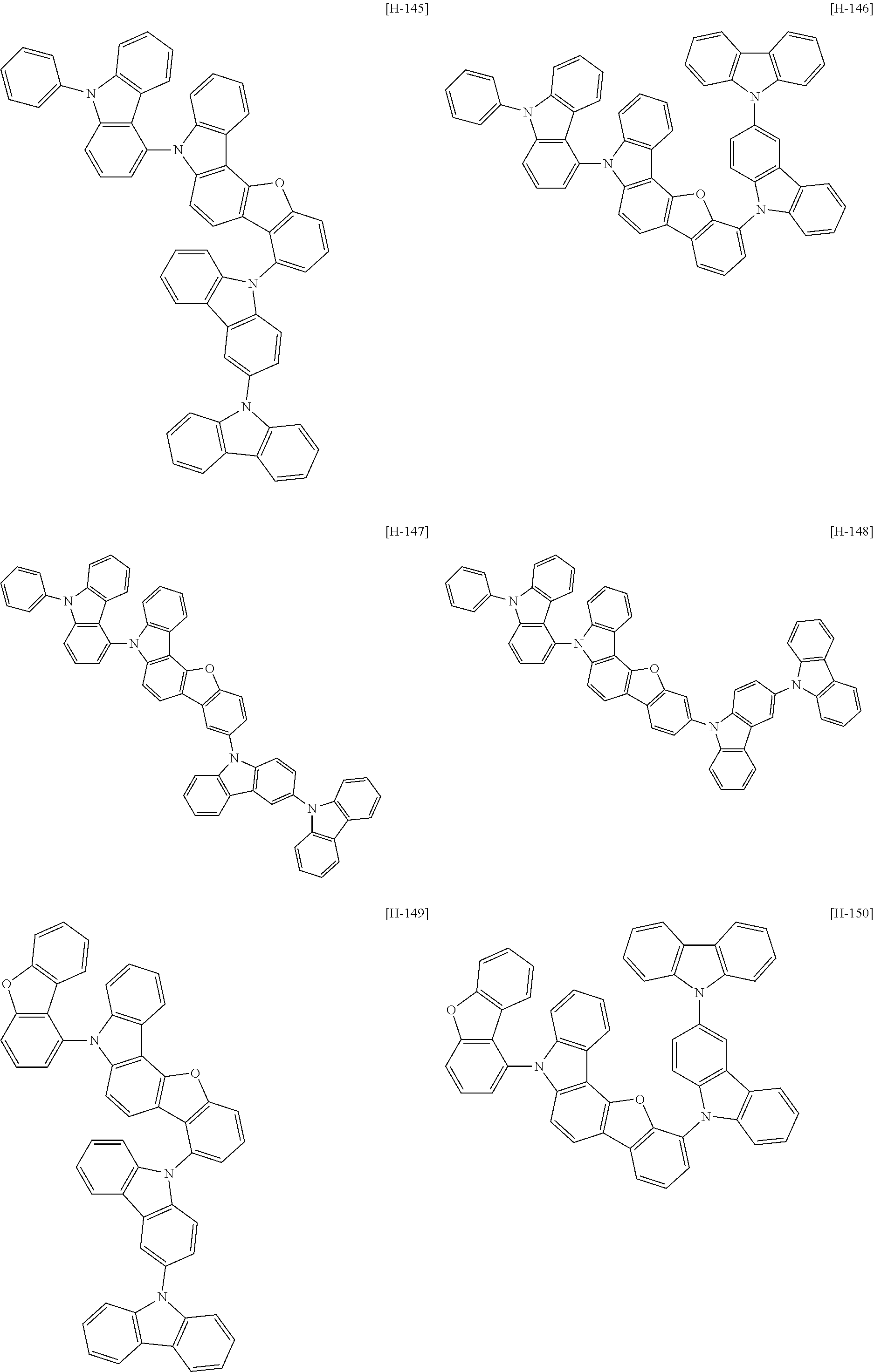

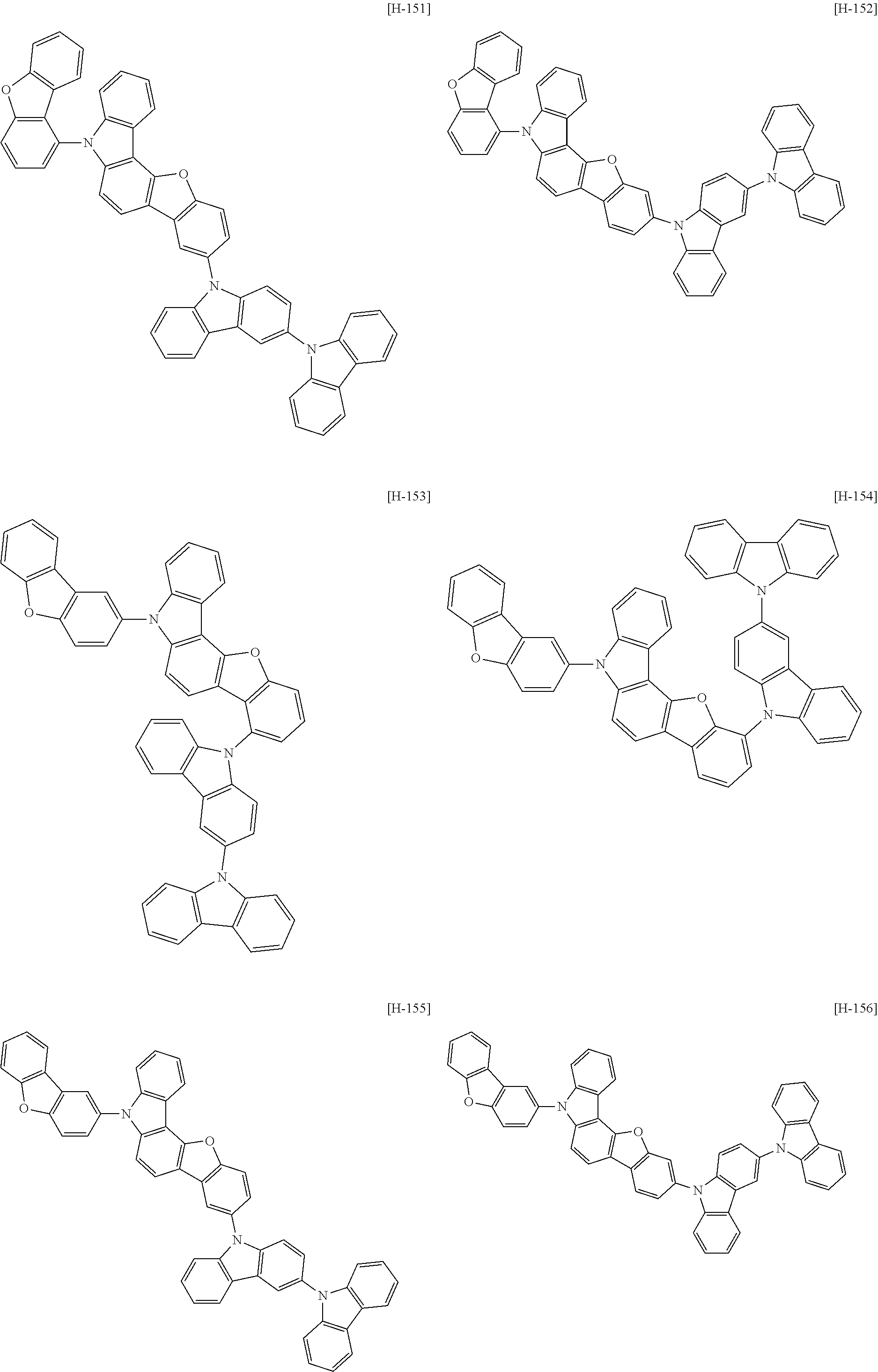

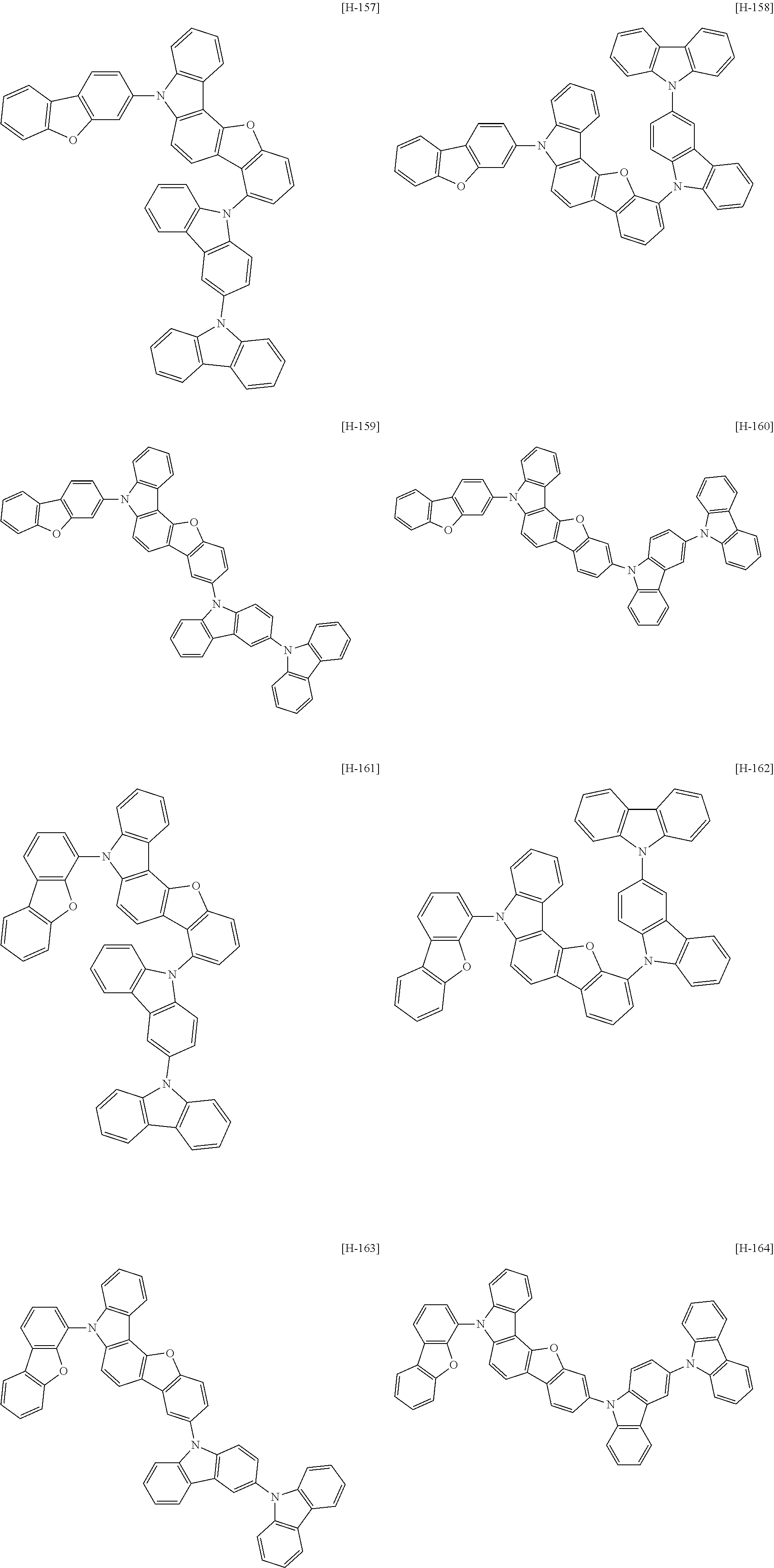

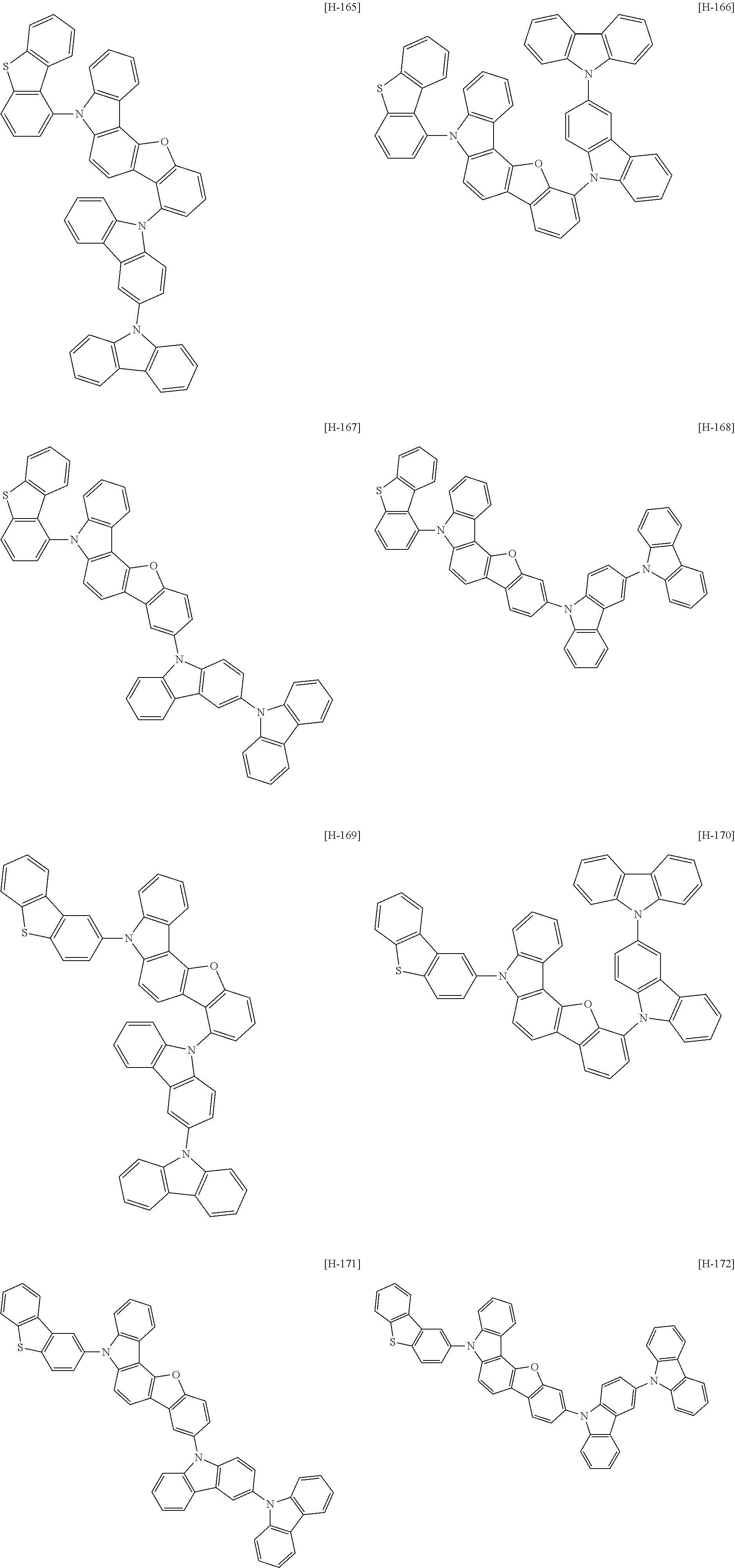

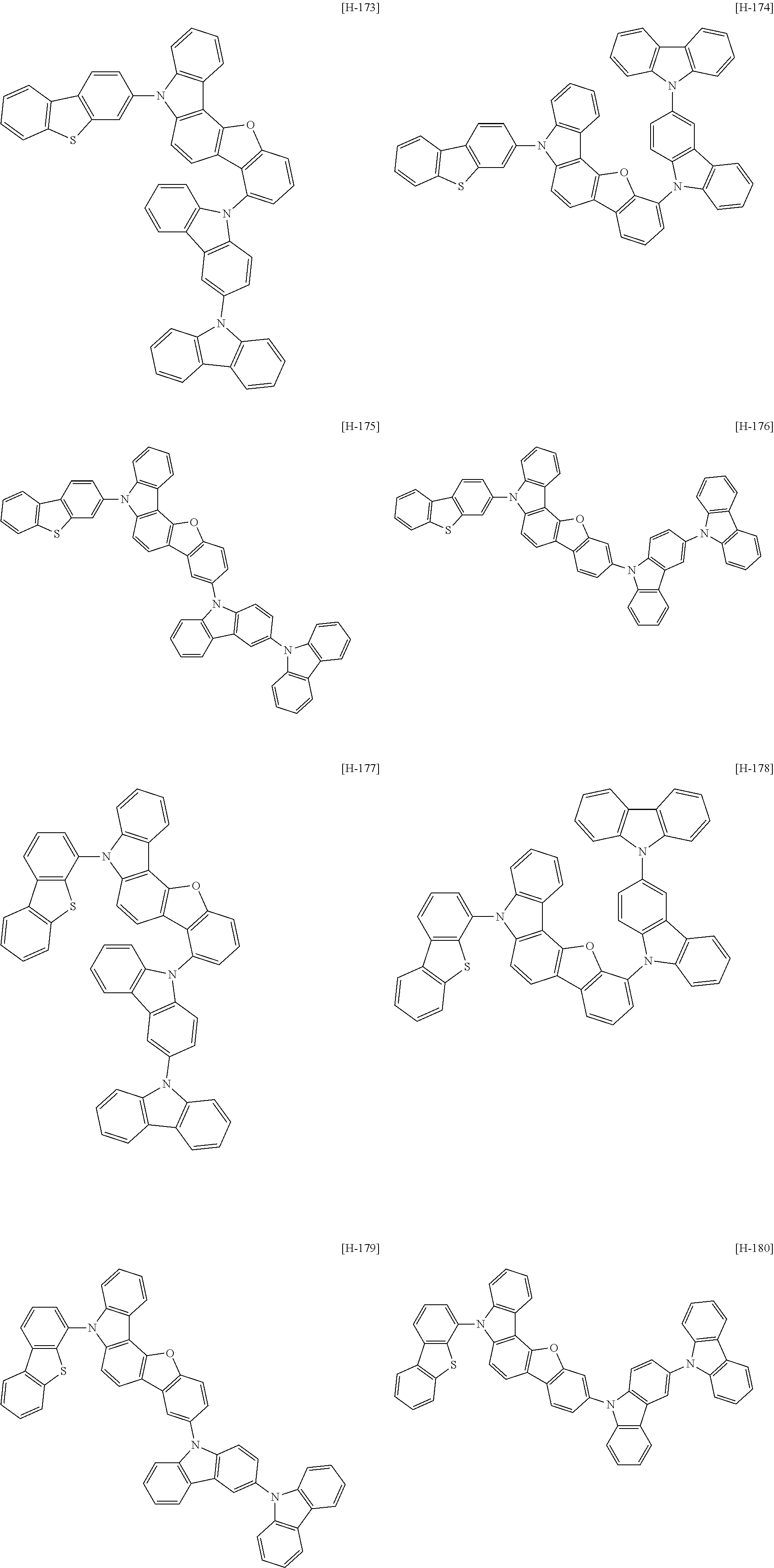

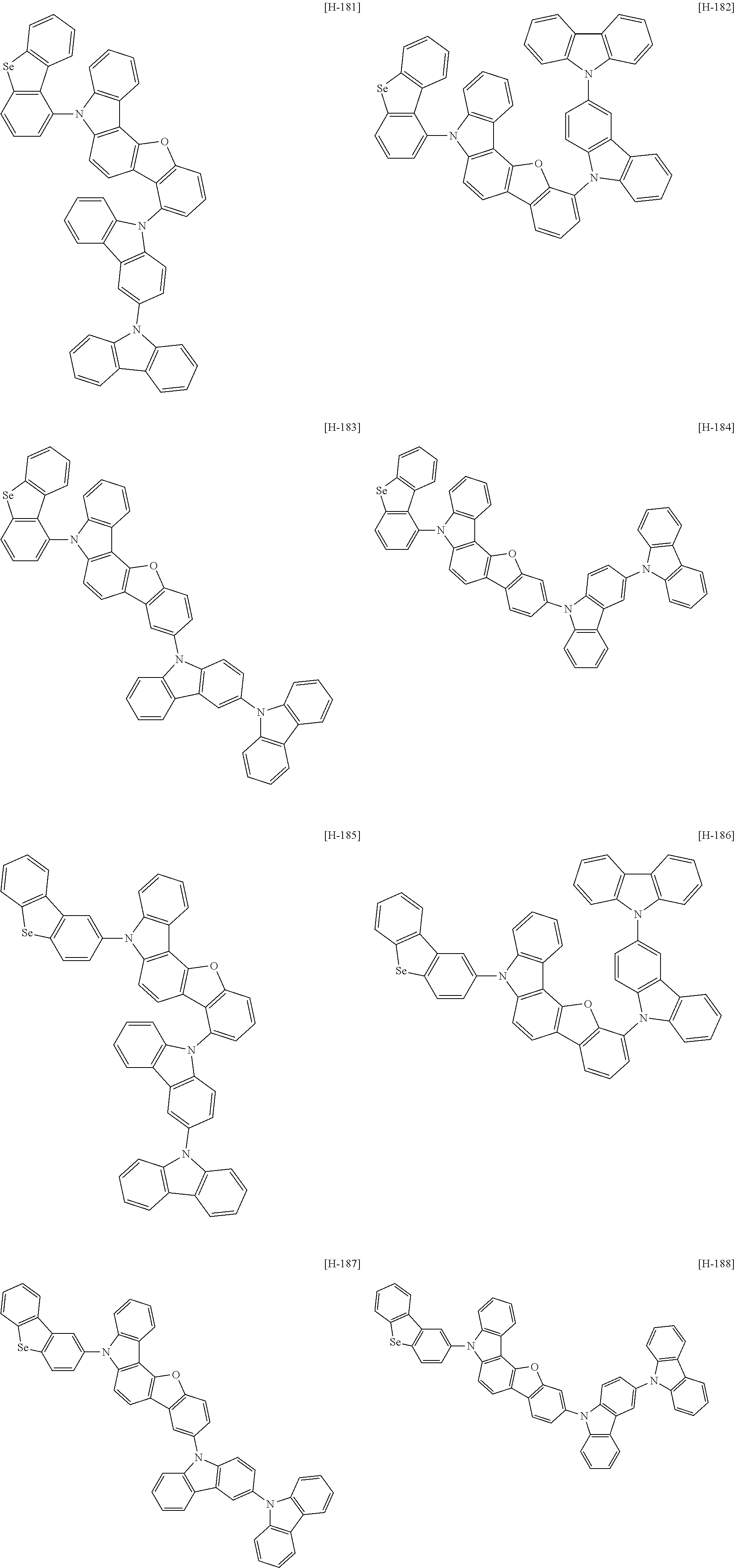

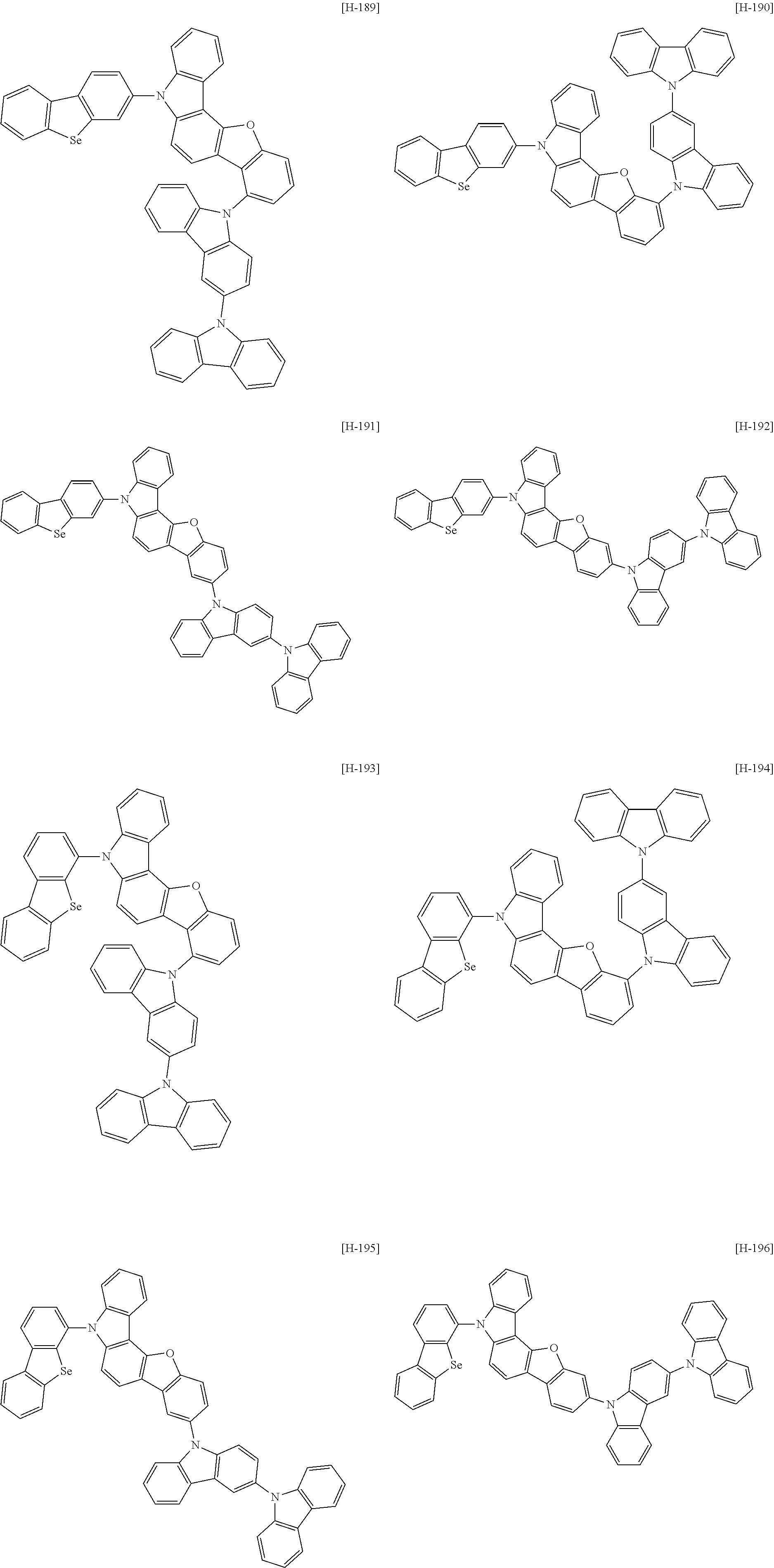

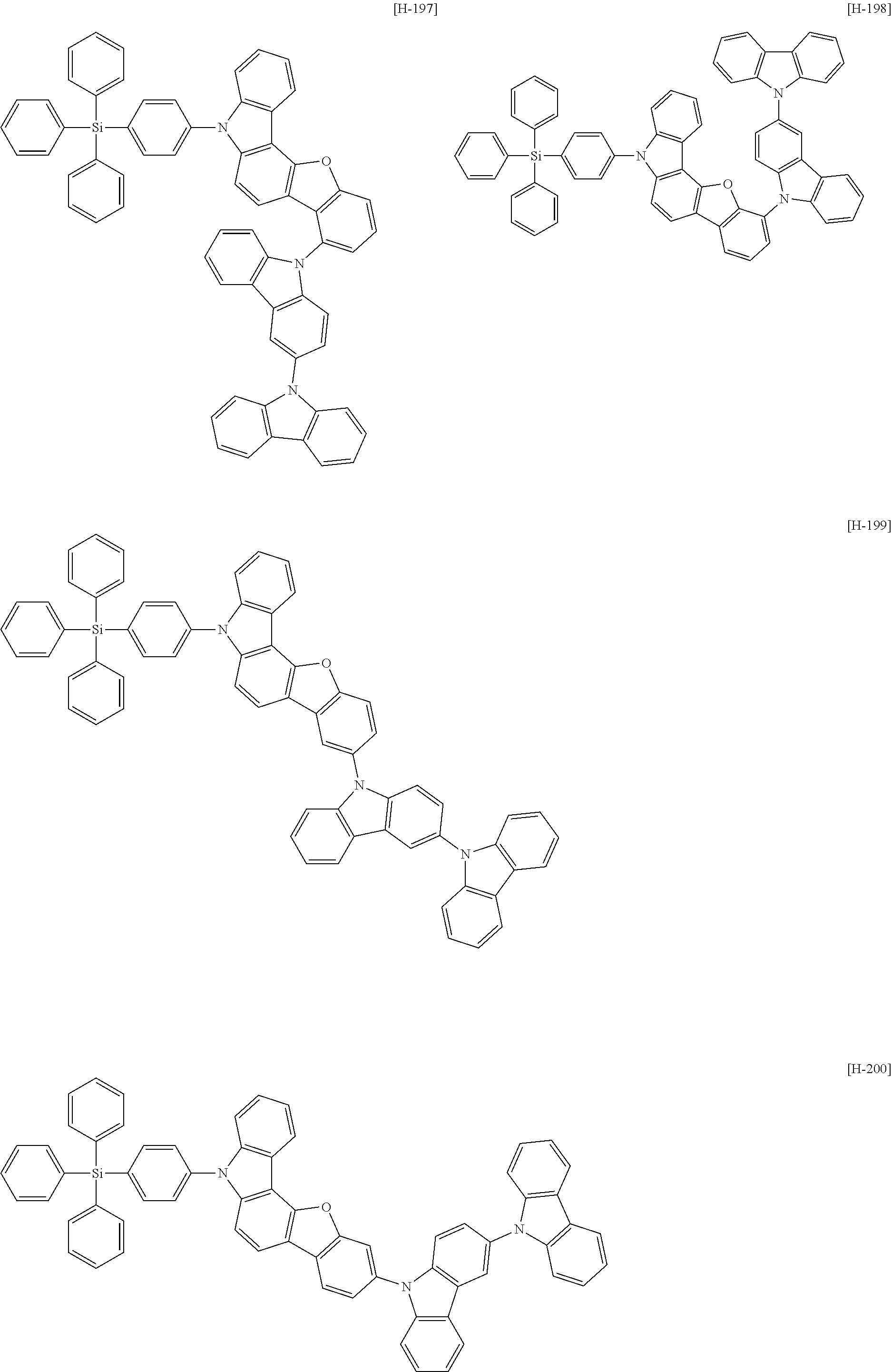

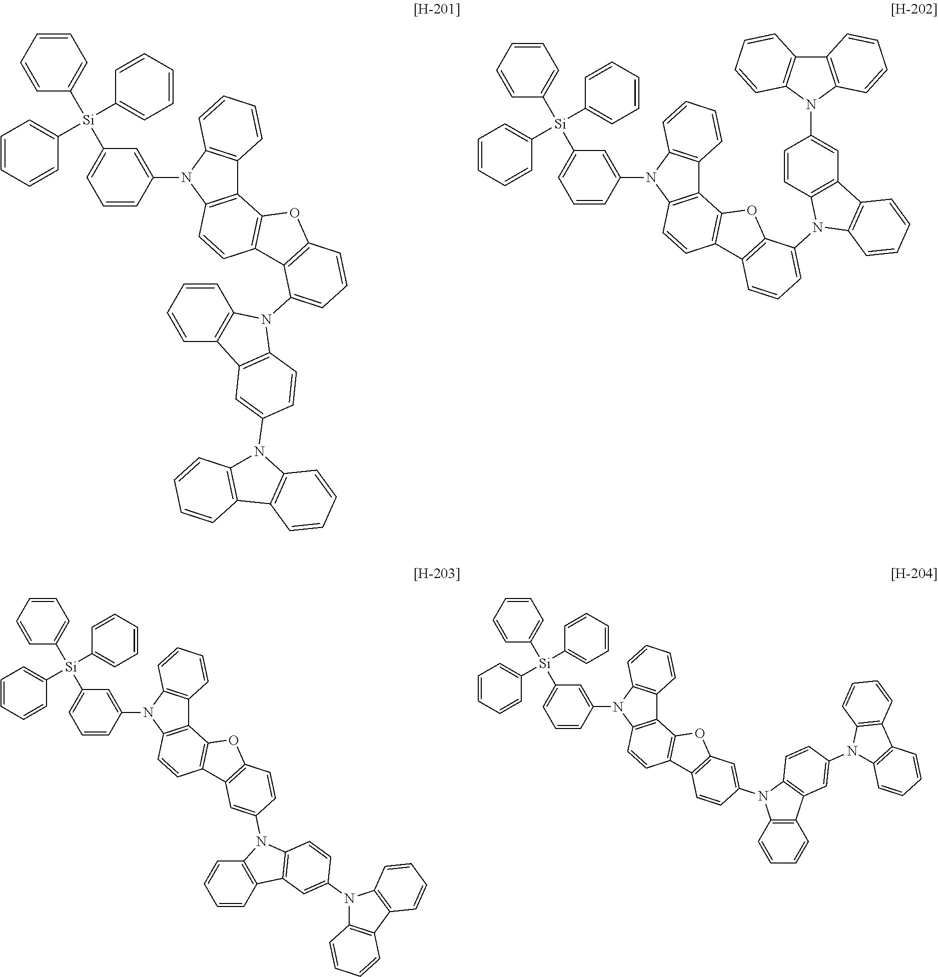

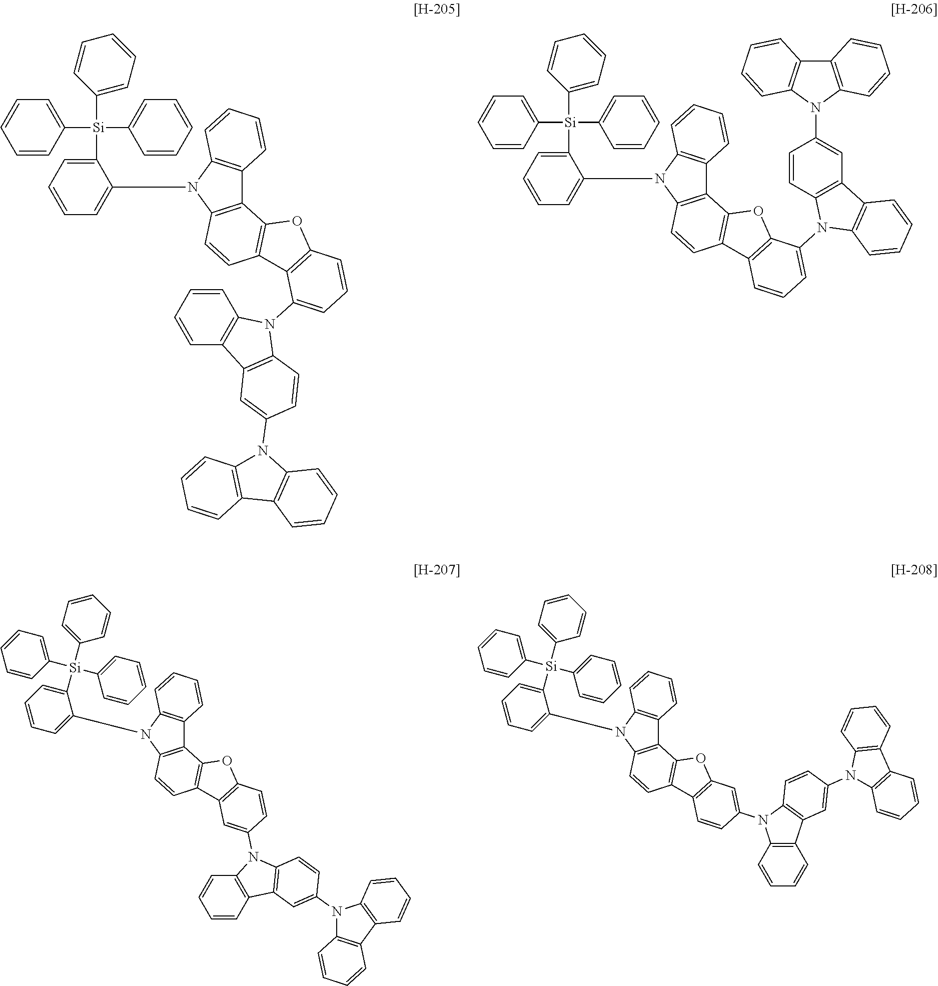 [SMILES: c1ccc([Si](c2ccccc2)(c2ccccc2)c2ccccc2-n2c3ccccc3c3c4oc5cccc(-n6c7ccccc7c7cc(-n8c9ccccc9c9ccccc98)ccc76)c5c4ccc32)cc1 ; c1ccc([Si](c2ccccc2)(c2ccccc2)c2ccccc2-n2c3ccccc3c3c4oc5c(-n6c7ccccc7c7cc(-n8c9ccccc9c9ccccc98)ccc76)cccc5c4ccc32)cc1 ; c1ccc([Si](c2ccccc2)(c2ccccc2)c2ccccc2-n2c3ccccc3c3c4oc5ccc(-n6c7ccccc7c7cc(-n8c9ccccc9c9ccccc98)ccc76)cc5c4ccc32)cc1 ; c1ccc([Si](c2ccccc2)(c2ccccc2)c2ccccc2-n2c3ccccc3c3c4oc5cc(-n6c7ccccc7c7cc(-n8c9ccccc9c9ccccc98)ccc76)ccc5c4ccc32)cc1]

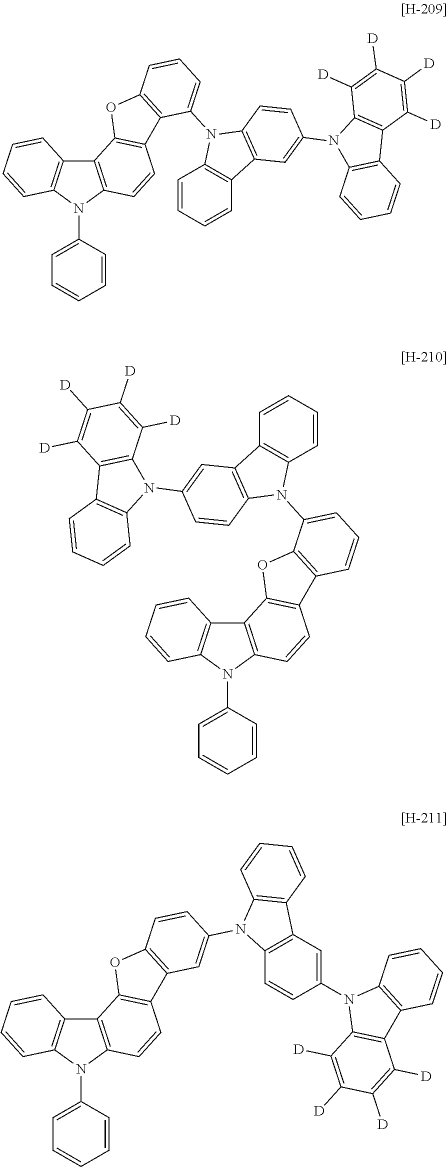 [SMILES: [2H]c1c([2H])c([2H])c2c(c1[2H])c1ccccc1n2-c1ccc2c(c1)c1ccccc1n2-c1cccc2oc3c(ccc4c3c3ccccc3n4-c3ccccc3)c12 ; [2H]c1c([2H])c([2H])c2c(c1[2H])c1ccccc1n2-c1ccc2c(c1)c1ccccc1n2-c1cccc2c1oc1c2ccc2c1c1ccccc1n2-c1ccccc1 ; [2H]c1c([2H])c([2H])c2c(c1[2H])c1ccccc1n2-c1ccc2c(c1)c1ccccc1n2-c1ccc2oc3c(ccc4c3c3ccccc3n4-c3ccccc3)c2c1]

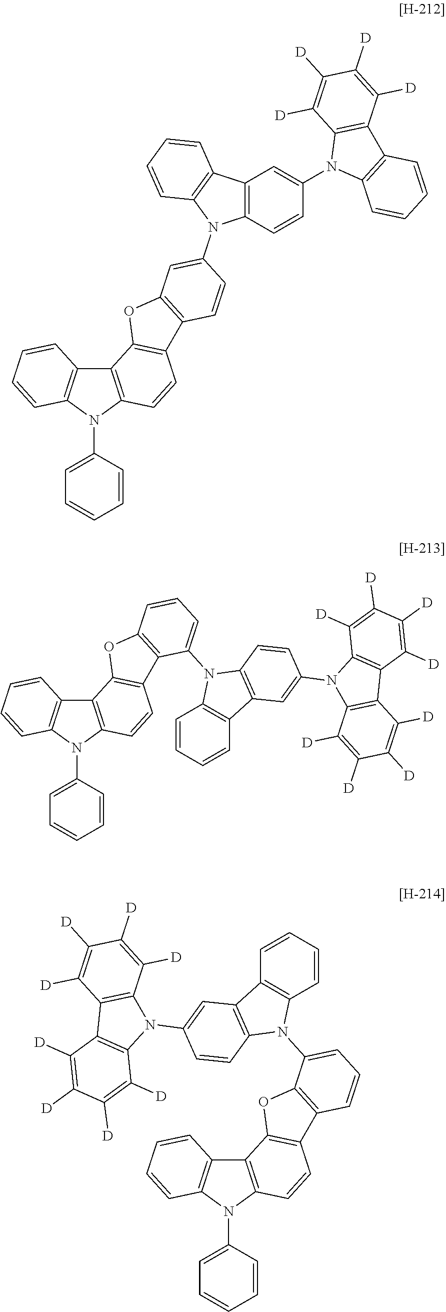 [SMILES: [2H]c1c([2H])c([2H])c2c(c1[2H])c1ccccc1n2-c1ccc2c(c1)c1ccccc1n2-c1ccc2c(c1)oc1c2ccc2c1c1ccccc1n2-c1ccccc1 ; [2H]c1c([2H])c([2H])c2c(c1[2H])c1c([2H])c([2H])c([2H])c([2H])c1n2-c1ccc2c(c1)c1ccccc1n2-c1cccc2oc3c(ccc4c3c3ccccc3n4-c3ccccc3)c12 ; [2H]c1c([2H])c([2H])c2c(c1[2H])c1c([2H])c([2H])c([2H])c([2H])c1n2-c1ccc2c(c1)c1ccccc1n2-c1cccc2c1oc1c2ccc2c1c1ccccc1n2-c1ccccc1]

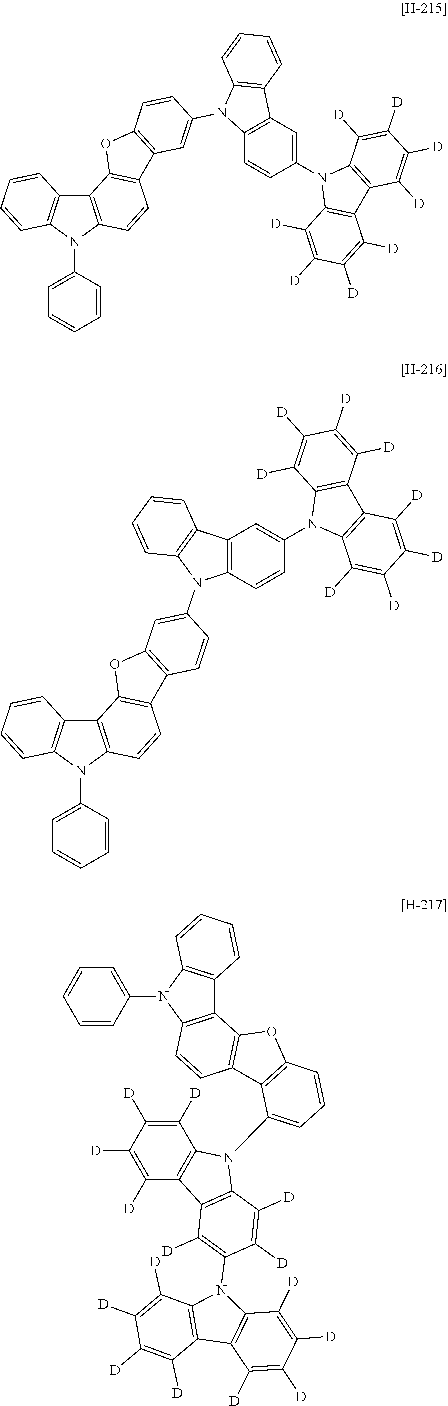 [SMILES: [2H]c1c([2H])c([2H])c2c(c1[2H])c1c([2H])c([2H])c([2H])c([2H])c1n2-c1ccc2c(c1)c1ccccc1n2-c1ccc2oc3c(ccc4c3c3ccccc3n4-c3ccccc3)c2c1 ; [2H]c1c([2H])c([2H])c2c(c1[2H])c1c([2H])c([2H])c([2H])c([2H])c1n2-c1ccc2c(c1)c1ccccc1n2-c1ccc2c(c1)oc1c2ccc2c1c1ccccc1n2-c1ccccc1 ; [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1cccc2oc3c(ccc4c3c3ccccc3n4-c3ccccc3)c12]

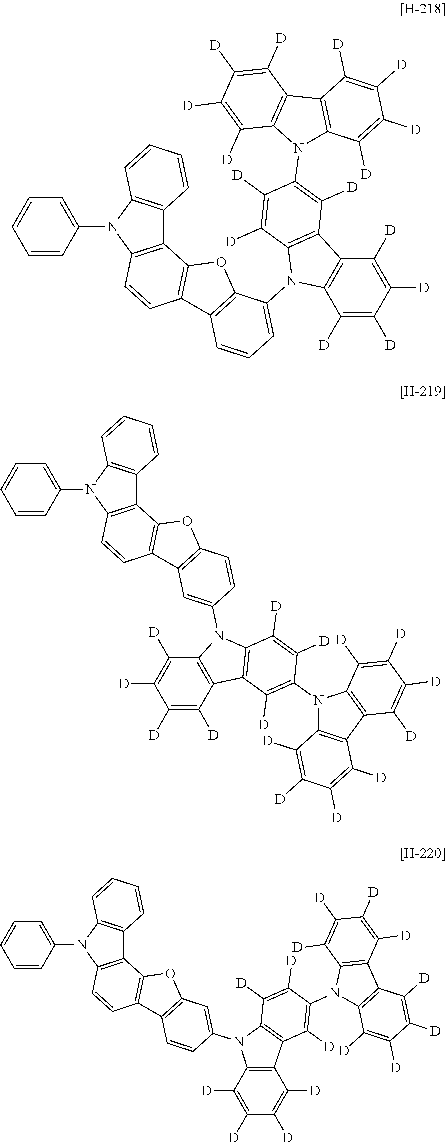

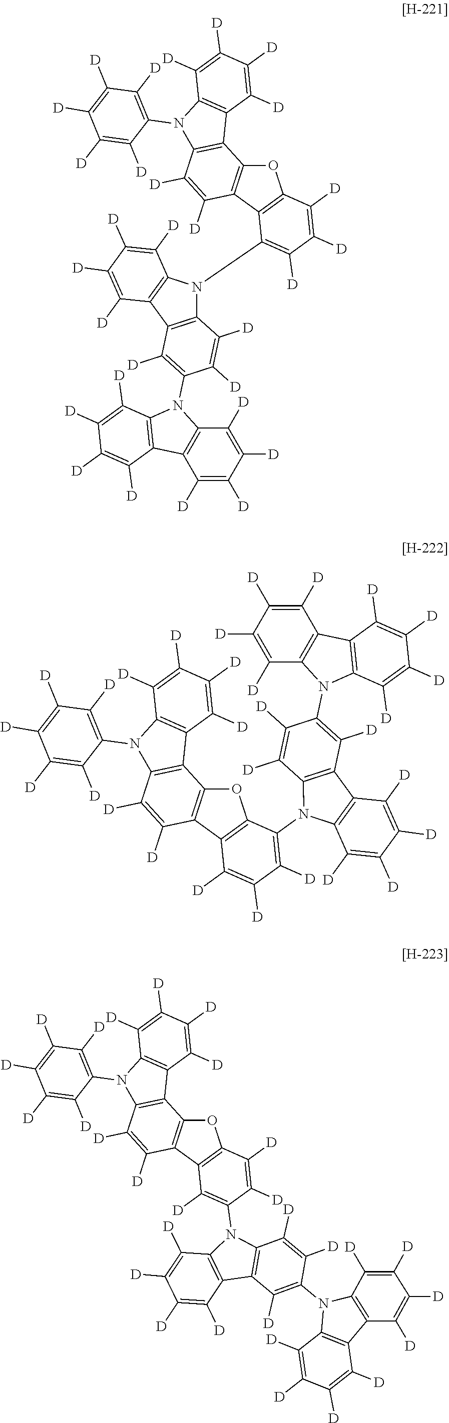

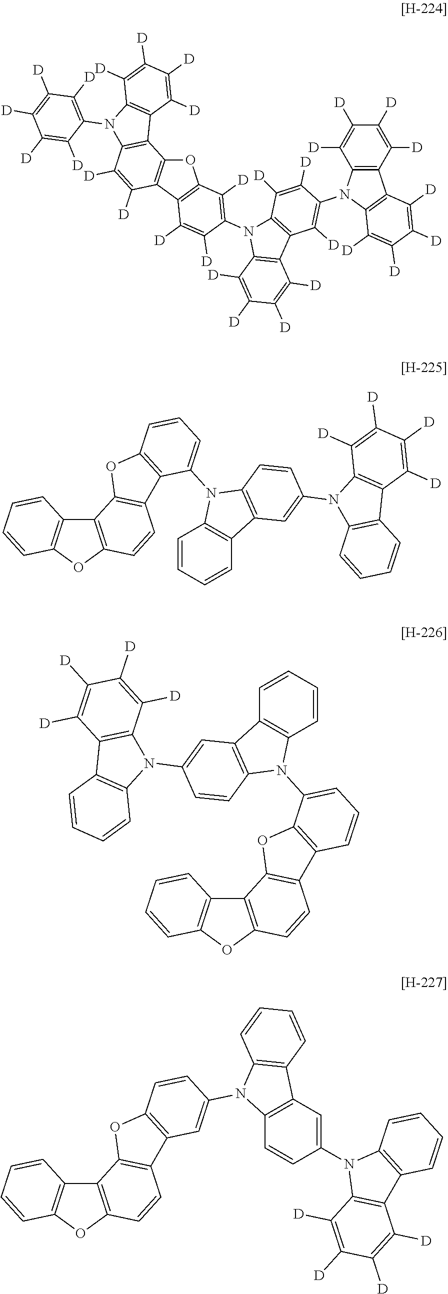

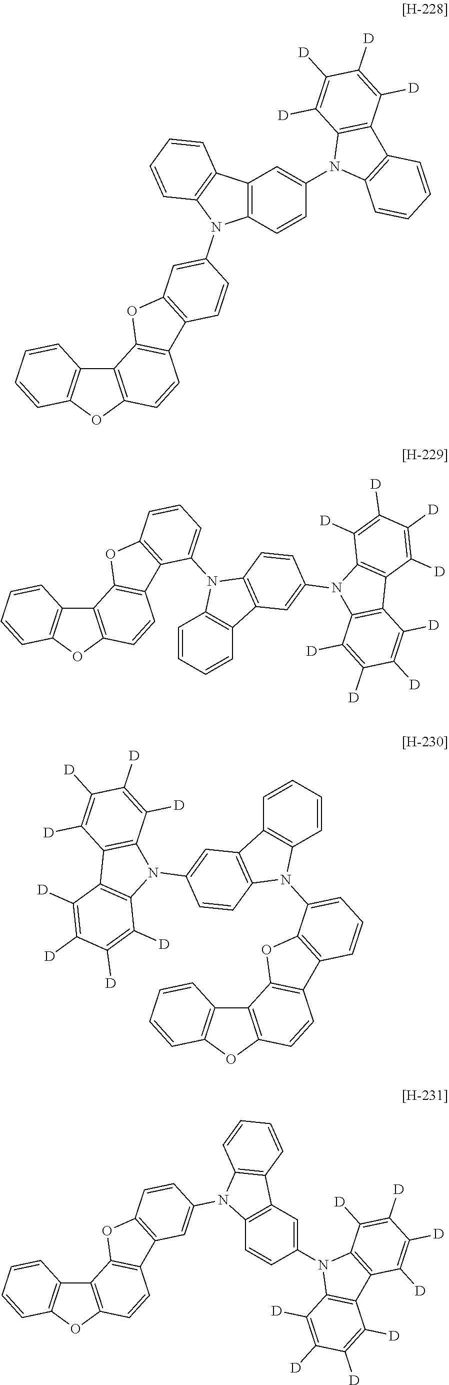 [SMILES: [2H]c1c([2H])c([2H])c2c(c1[2H])c1ccccc1n2-c1ccc2c(c1)c1ccccc1n2-c1ccc2c(c1)oc1c2ccc2oc3ccccc3c21 ; [2H]c1c([2H])c([2H])c2c(c1[2H])c1c([2H])c([2H])c([2H])c([2H])c1n2-c1ccc2c(c1)c1ccccc1n2-c1cccc2oc3c(ccc4oc5ccccc5c43)c12 ; [2H]c1c([2H])c([2H])c2c(c1[2H])c1c([2H])c([2H])c([2H])c([2H])c1n2-c1ccc2c(c1)c1ccccc1n2-c1cccc2c1oc1c2ccc2oc3ccccc3c21 ; [2H]c1c([2H])c([2H])c2c(c1[2H])c1c([2H])c([2H])c([2H])c([2H])c1n2-c1ccc2c(c1)c1ccccc1n2-c1ccc2oc3c(ccc4oc5ccccc5c43)c2c1]

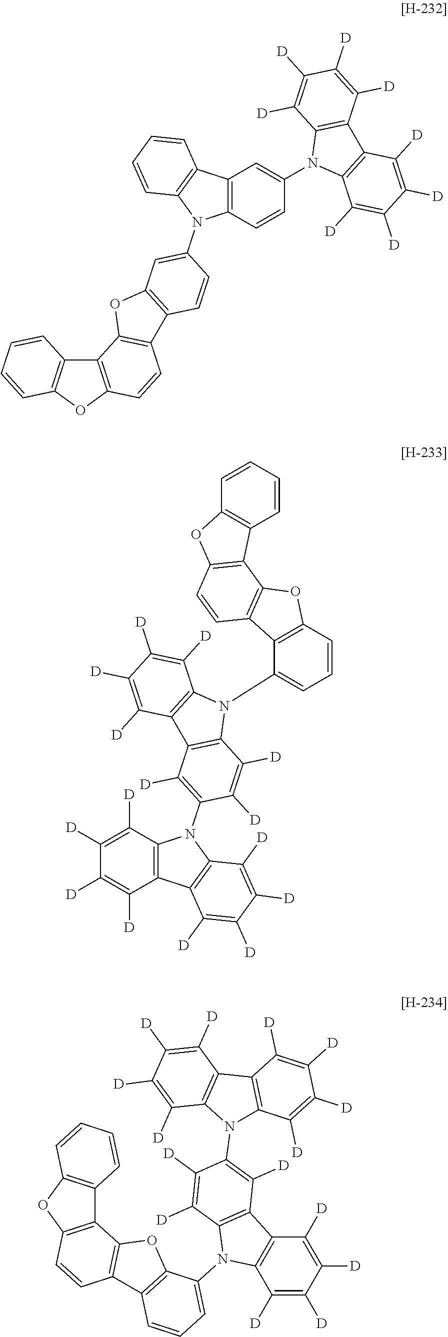 [SMILES: [2H]c1c([2H])c([2H])c2c(c1[2H])c1c([2H])c([2H])c([2H])c([2H])c1n2-c1ccc2c(c1)c1ccccc1n2-c1ccc2c(c1)oc1c2ccc2oc3ccccc3c21 ; [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1cccc2oc3c(ccc4oc5ccccc5c43)c12 ; [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1cccc2c1oc1c2ccc2oc3ccccc3c21]

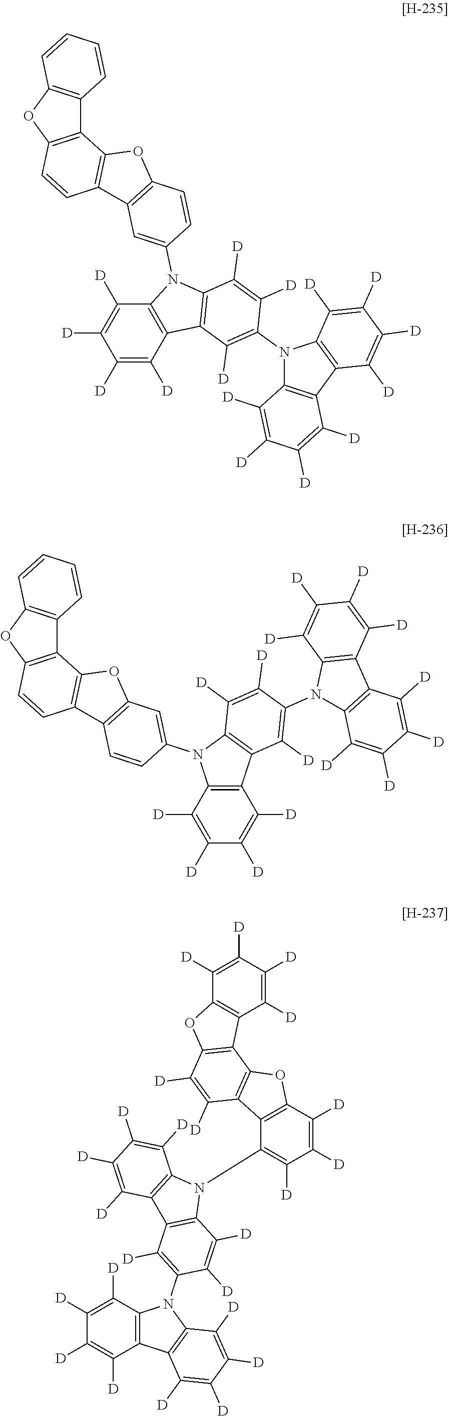

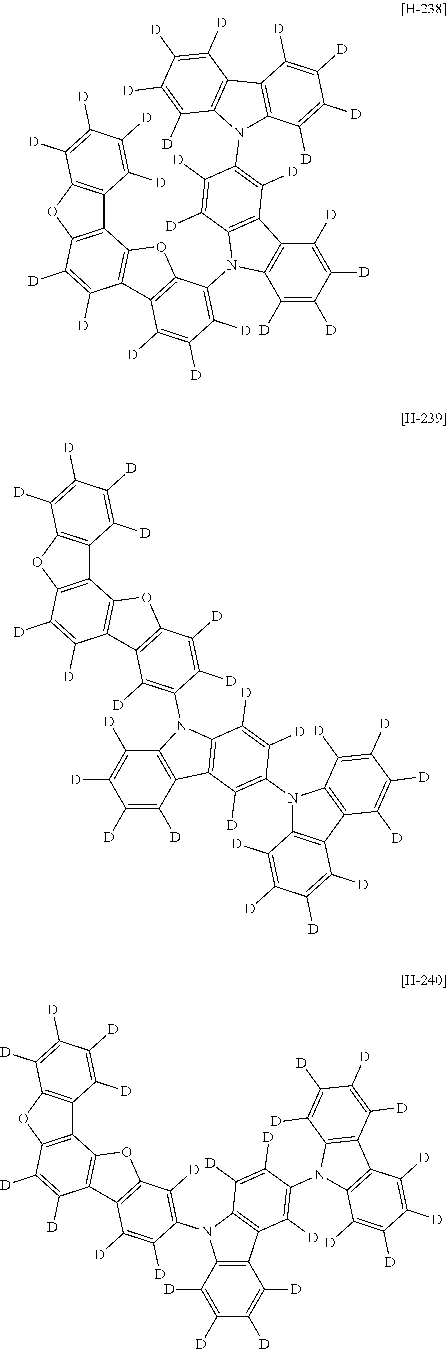

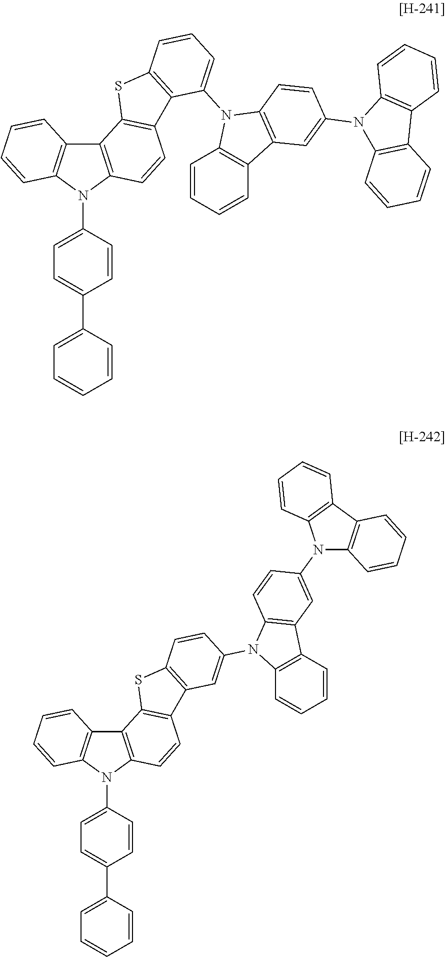 [SMILES: c1ccc(-c2ccc(-n3c4ccccc4c4c5sc6cccc(-n7c8ccccc8c8cc(-n9c%10ccccc%10c%10ccccc%109)ccc87)c6c5ccc43)cc2)cc1 ; c1ccc(-c2ccc(-n3c4ccccc4c4c5sc6ccc(-n7c8ccccc8c8cc(-n9c%10ccccc%10c%10ccccc%109)ccc87)cc6c5ccc43)cc2)cc1]

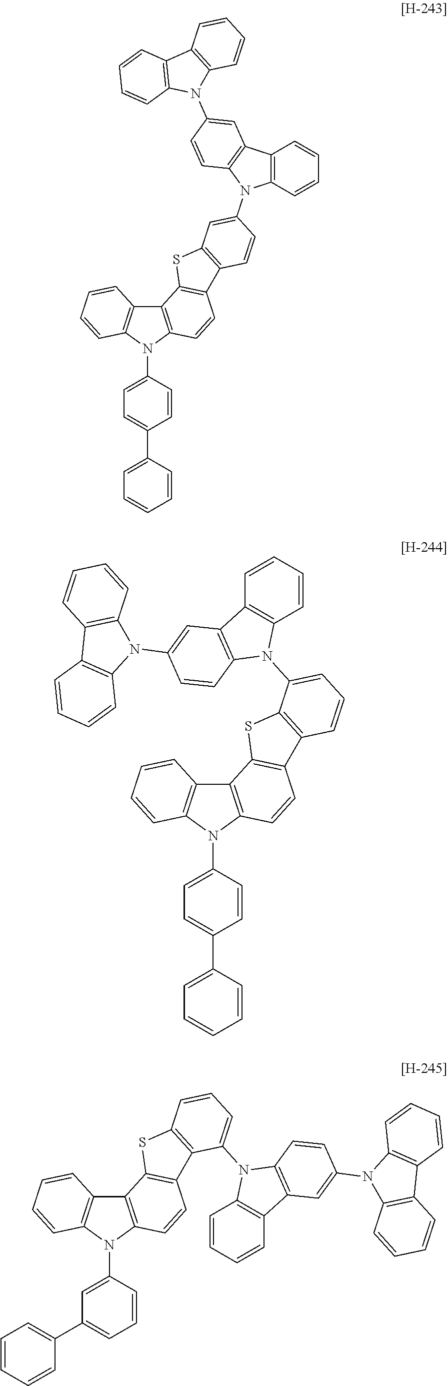 [SMILES: c1ccc(-c2ccc(-n3c4ccccc4c4c5sc6cc(-n7c8ccccc8c8cc(-n9c%10ccccc%10c%10ccccc%109)ccc87)ccc6c5ccc43)cc2)cc1 ; c1ccc(-c2ccc(-n3c4ccccc4c4c5sc6c(-n7c8ccccc8c8cc(-n9c%10ccccc%10c%10ccccc%109)ccc87)cccc6c5ccc43)cc2)cc1 ; c1ccc(-c2cccc(-n3c4ccccc4c4c5sc6cccc(-n7c8ccccc8c8cc(-n9c%10ccccc%10c%10ccccc%109)ccc87)c6c5ccc43)c2)cc1]

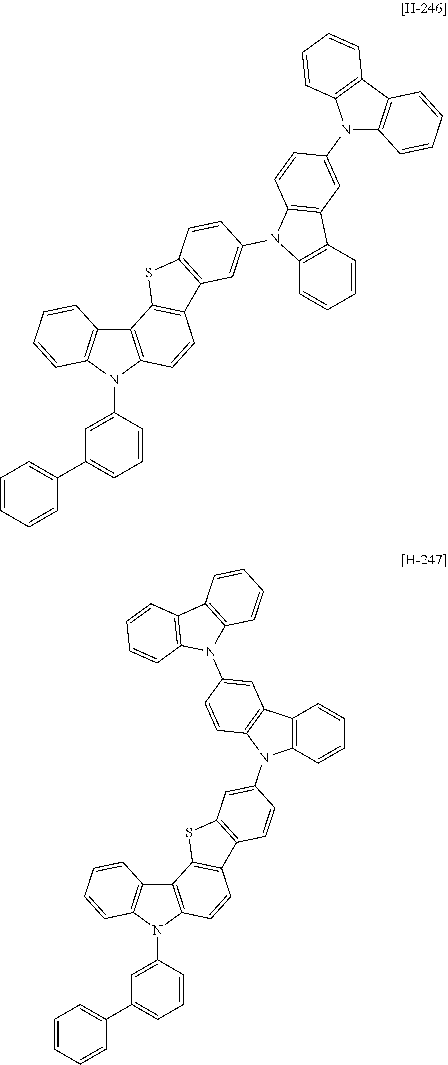 [SMILES: c1ccc(-c2cccc(-n3c4ccccc4c4c5sc6ccc(-n7c8ccccc8c8cc(-n9c%10ccccc%10c%10ccccc%109)ccc87)cc6c5ccc43)c2)cc1 ; c1ccc(-c2cccc(-n3c4ccccc4c4c5sc6cc(-n7c8ccccc8c8cc(-n9c%10ccccc%10c%10ccccc%109)ccc87)ccc6c5ccc43)c2)cc1]

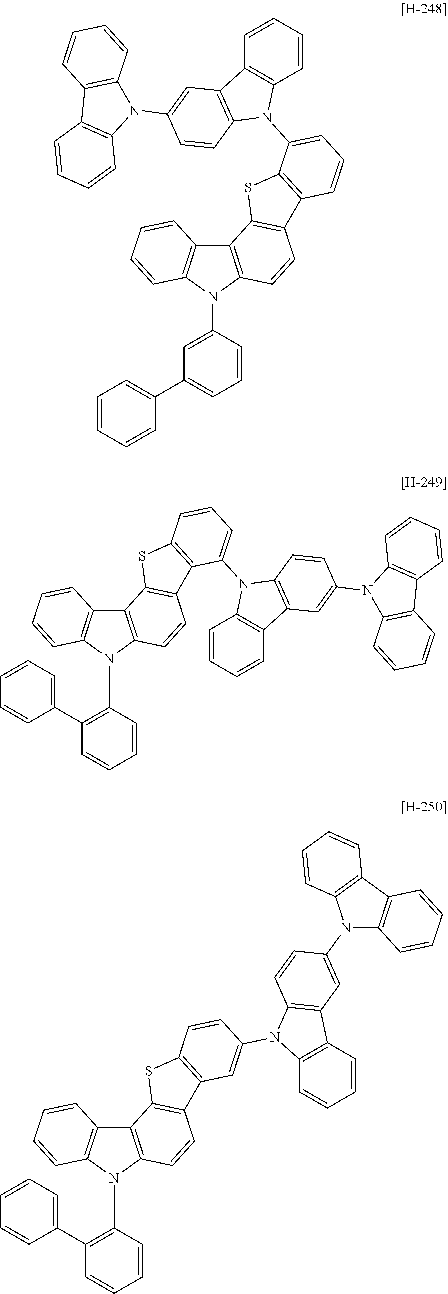 [SMILES: c1ccc(-c2cccc(-n3c4ccccc4c4c5sc6c(-n7c8ccccc8c8cc(-n9c%10ccccc%10c%10ccccc%109)ccc87)cccc6c5ccc43)c2)cc1 ; c1ccc(-c2ccccc2-n2c3ccccc3c3c4sc5cccc(-n6c7ccccc7c7cc(-n8c9ccccc9c9ccccc98)ccc76)c5c4ccc32)cc1 ; c1ccc(-c2ccccc2-n2c3ccccc3c3c4sc5ccc(-n6c7ccccc7c7cc(-n8c9ccccc9c9ccccc98)ccc76)cc5c4ccc32)cc1]

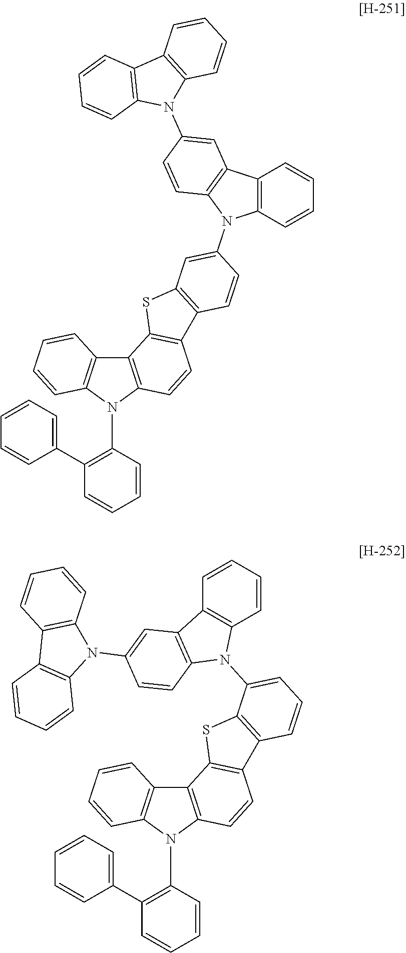 [SMILES: c1ccc(-c2ccccc2-n2c3ccccc3c3c4sc5cc(-n6c7ccccc7c7cc(-n8c9ccccc9c9ccccc98)ccc76)ccc5c4ccc32)cc1 ; c1ccc(-c2ccccc2-n2c3ccccc3c3c4sc5c(-n6c7ccccc7c7cc(-n8c9ccccc9c9ccccc98)ccc76)cccc5c4ccc32)cc1]

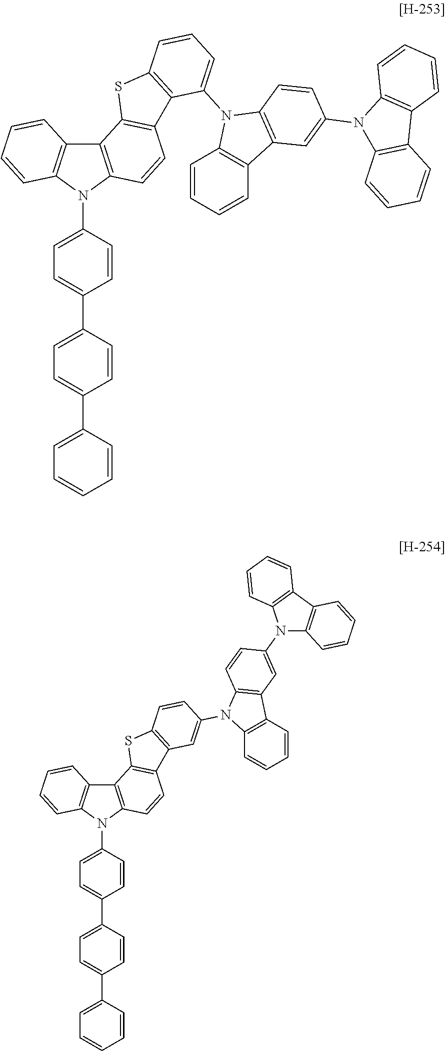 [SMILES: c1ccc(-c2ccc(-c3ccc(-n4c5ccccc5c5c6sc7cccc(-n8c9ccccc9c9cc(-n%10c%11ccccc%11c%11ccccc%11%10)ccc98)c7c6ccc54)cc3)cc2)cc1 ; c1ccc(-c2ccc(-c3ccc(-n4c5ccccc5c5c6sc7ccc(-n8c9ccccc9c9cc(-n%10c%11ccccc%11c%11ccccc%11%10)ccc98)cc7c6ccc54)cc3)cc2)cc1]

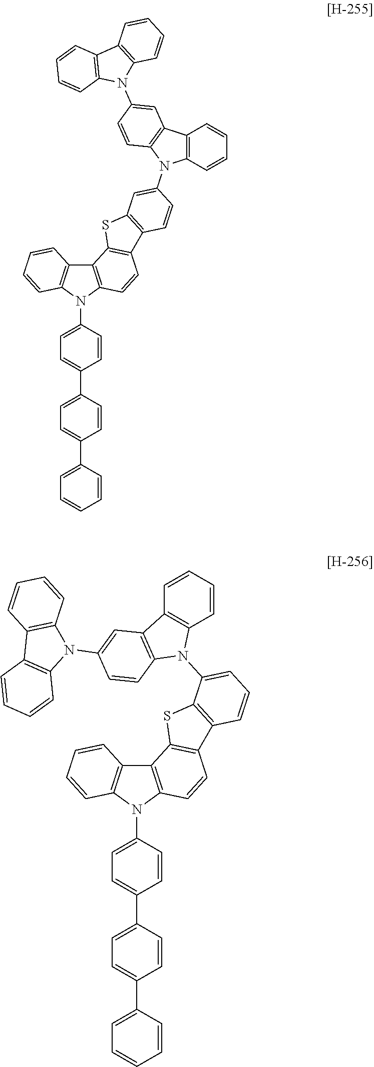 [SMILES: c1ccc(-c2ccc(-c3ccc(-n4c5ccccc5c5c6sc7cc(-n8c9ccccc9c9cc(-n%10c%11ccccc%11c%11ccccc%11%10)ccc98)ccc7c6ccc54)cc3)cc2)cc1 ; c1ccc(-c2ccc(-c3ccc(-n4c5ccccc5c5c6sc7c(-n8c9ccccc9c9cc(-n%10c%11ccccc%11c%11ccccc%11%10)ccc98)cccc7c6ccc54)cc3)cc2)cc1]

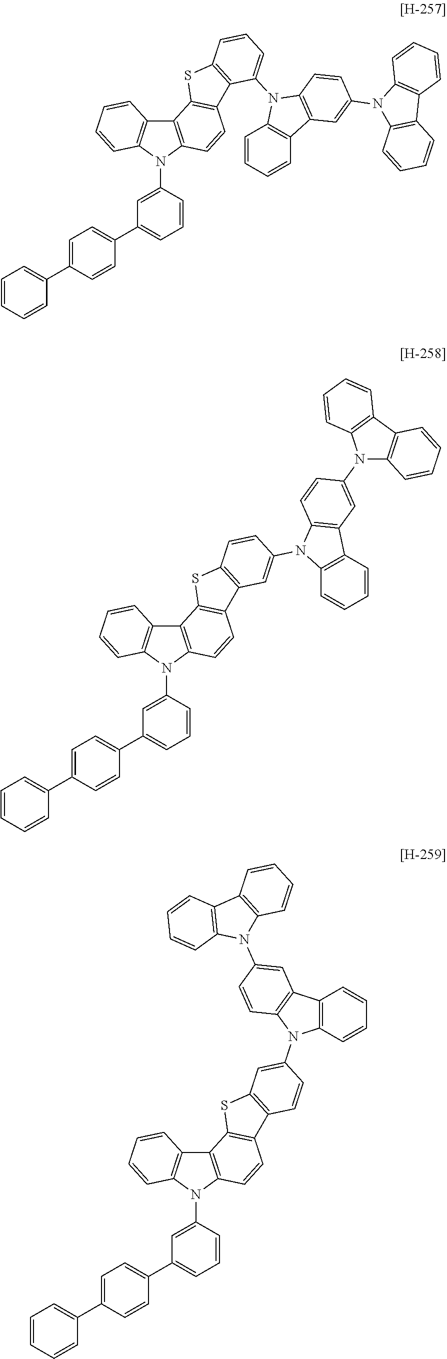 [SMILES: c1ccc(-c2ccc(-c3cccc(-n4c5ccccc5c5c6sc7cccc(-n8c9ccccc9c9cc(-n%10c%11ccccc%11c%11ccccc%11%10)ccc98)c7c6ccc54)c3)cc2)cc1 ; c1ccc(-c2ccc(-c3cccc(-n4c5ccccc5c5c6sc7ccc(-n8c9ccccc9c9cc(-n%10c%11ccccc%11c%11ccccc%11%10)ccc98)cc7c6ccc54)c3)cc2)cc1 ; c1ccc(-c2ccc(-c3cccc(-n4c5ccccc5c5c6sc7cc(-n8c9ccccc9c9cc(-n%10c%11ccccc%11c%11ccccc%11%10)ccc98)ccc7c6ccc54)c3)cc2)cc1]

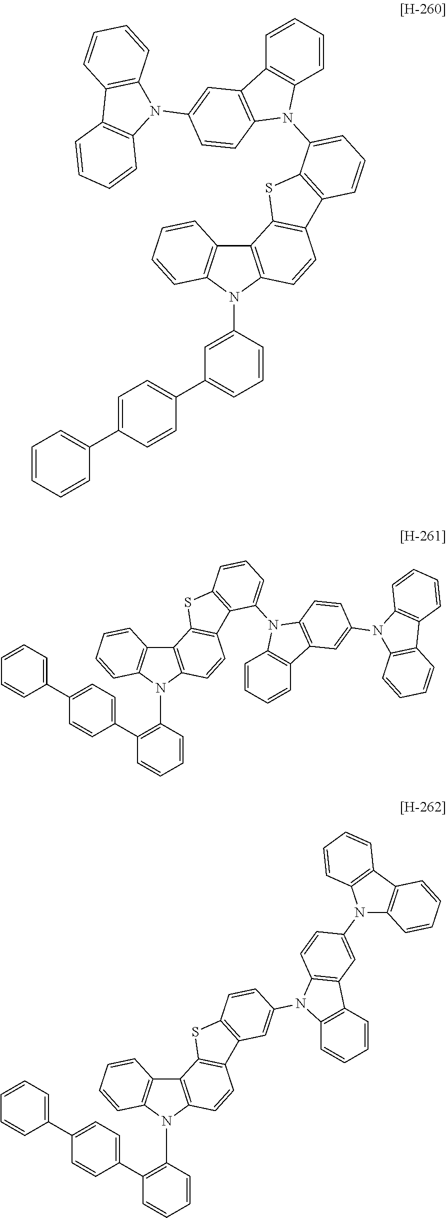 [SMILES: c1ccc(-c2ccc(-c3cccc(-n4c5ccccc5c5c6sc7c(-n8c9ccccc9c9cc(-n%10c%11ccccc%11c%11ccccc%11%10)ccc98)cccc7c6ccc54)c3)cc2)cc1 ; c1ccc(-c2ccc(-c3ccccc3-n3c4ccccc4c4c5sc6cccc(-n7c8ccccc8c8cc(-n9c%10ccccc%10c%10ccccc%109)ccc87)c6c5ccc43)cc2)cc1 ; c1ccc(-c2ccc(-c3ccccc3-n3c4ccccc4c4c5sc6ccc(-n7c8ccccc8c8cc(-n9c%10ccccc%10c%10ccccc%109)ccc87)cc6c5ccc43)cc2)cc1]

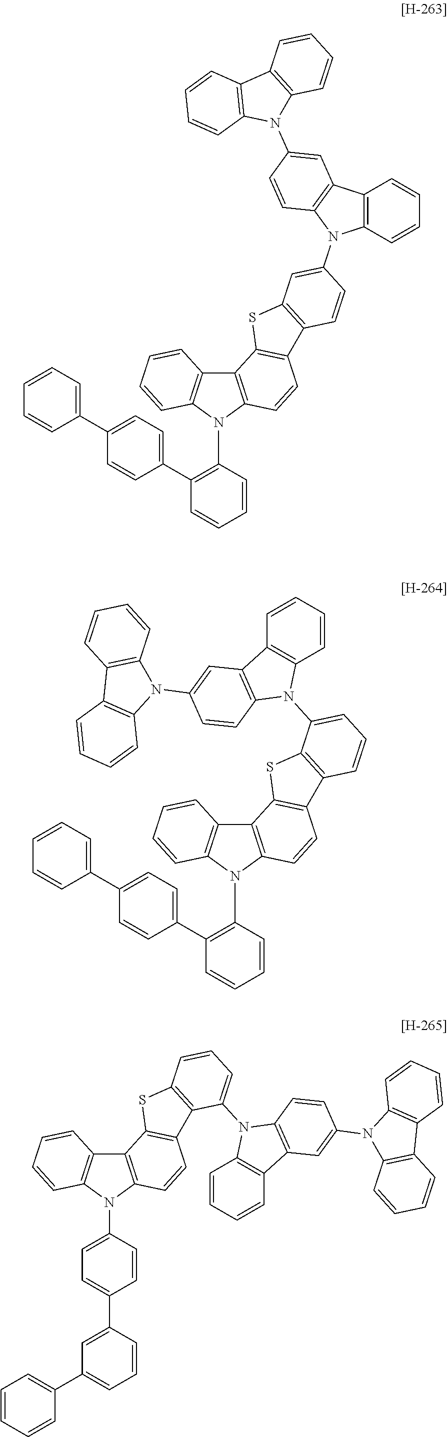 [SMILES: c1ccc(-c2ccc(-c3ccccc3-n3c4ccccc4c4c5sc6cc(-n7c8ccccc8c8cc(-n9c%10ccccc%10c%10ccccc%109)ccc87)ccc6c5ccc43)cc2)cc1 ; c1ccc(-c2ccc(-c3ccccc3-n3c4ccccc4c4c5sc6c(-n7c8ccccc8c8cc(-n9c%10ccccc%10c%10ccccc%109)ccc87)cccc6c5ccc43)cc2)cc1 ; c1ccc(-c2cccc(-c3ccc(-n4c5ccccc5c5c6sc7cccc(-n8c9ccccc9c9cc(-n%10c%11ccccc%11c%11ccccc%11%10)ccc98)c7c6ccc54)cc3)c2)cc1]

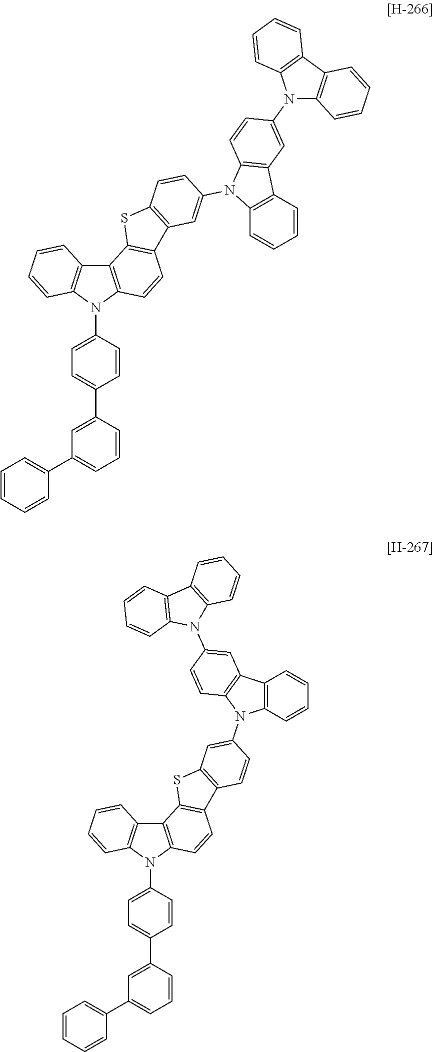 [SMILES: c1ccc(-c2cccc(-c3ccc(-n4c5ccccc5c5c6sc7ccc(-n8c9ccccc9c9cc(-n%10c%11ccccc%11c%11ccccc%11%10)ccc98)cc7c6ccc54)cc3)c2)cc1 ; c1ccc(-c2cccc(-c3ccc(-n4c5ccccc5c5c6sc7cc(-n8c9ccccc9c9cc(-n%10c%11ccccc%11c%11ccccc%11%10)ccc98)ccc7c6ccc54)cc3)c2)cc1]

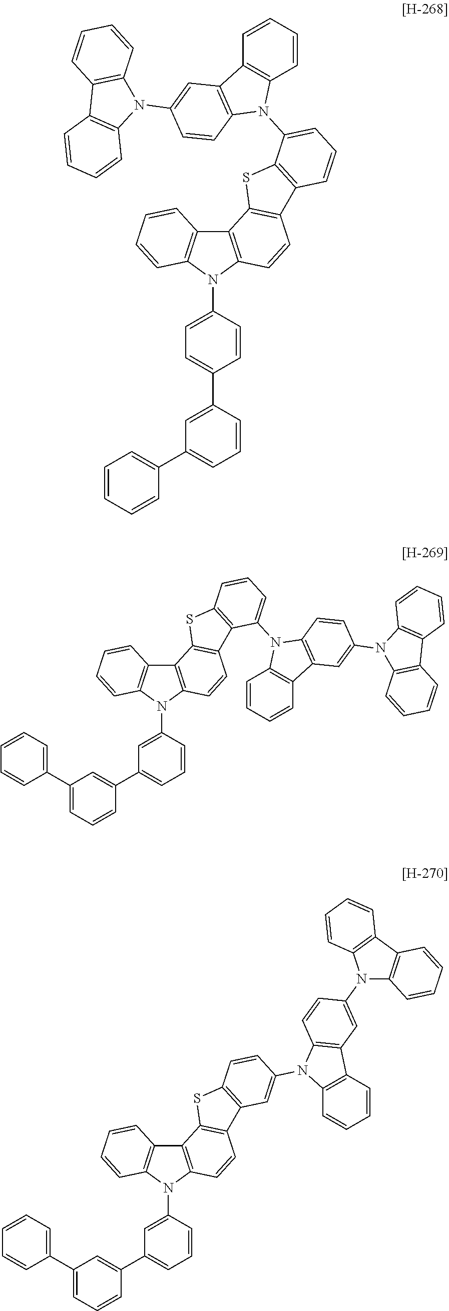 [SMILES: c1ccc(-c2cccc(-c3ccc(-n4c5ccccc5c5c6sc7c(-n8c9ccccc9c9cc(-n%10c%11ccccc%11c%11ccccc%11%10)ccc98)cccc7c6ccc54)cc3)c2)cc1 ; c1ccc(-c2cccc(-c3cccc(-n4c5ccccc5c5c6sc7cccc(-n8c9ccccc9c9cc(-n%10c%11ccccc%11c%11ccccc%11%10)ccc98)c7c6ccc54)c3)c2)cc1 ; c1ccc(-c2cccc(-c3cccc(-n4c5ccccc5c5c6sc7ccc(-n8c9ccccc9c9cc(-n%10c%11ccccc%11c%11ccccc%11%10)ccc98)cc7c6ccc54)c3)c2)cc1]

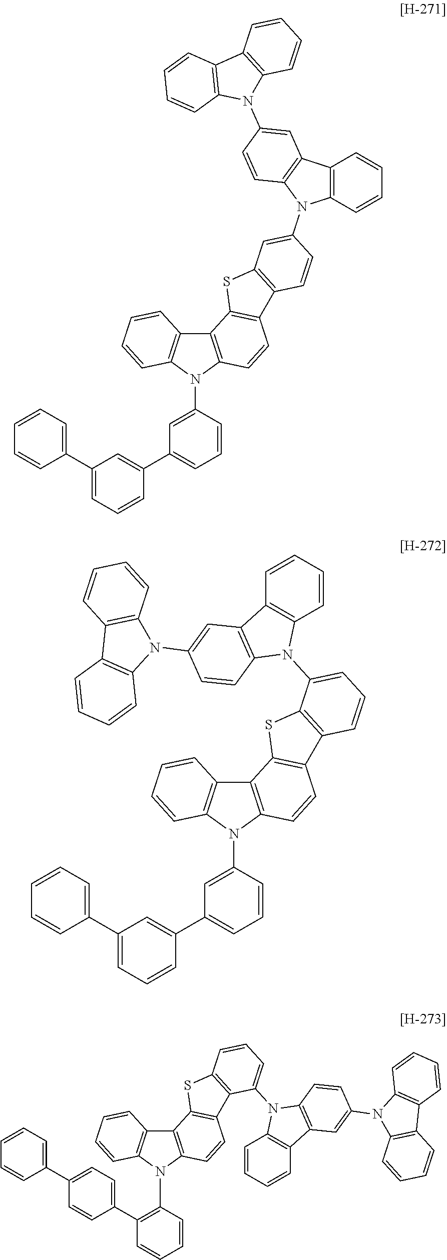 [SMILES: c1ccc(-c2cccc(-c3cccc(-n4c5ccccc5c5c6sc7cc(-n8c9ccccc9c9cc(-n%10c%11ccccc%11c%11ccccc%11%10)ccc98)ccc7c6ccc54)c3)c2)cc1 ; c1ccc(-c2cccc(-c3cccc(-n4c5ccccc5c5c6sc7c(-n8c9ccccc9c9cc(-n%10c%11ccccc%11c%11ccccc%11%10)ccc98)cccc7c6ccc54)c3)c2)cc1 ; c1ccc(-c2ccc(-c3ccccc3-n3c4ccccc4c4c5sc6cccc(-n7c8ccccc8c8cc(-n9c%10ccccc%10c%10ccccc%109)ccc87)c6c5ccc43)cc2)cc1]

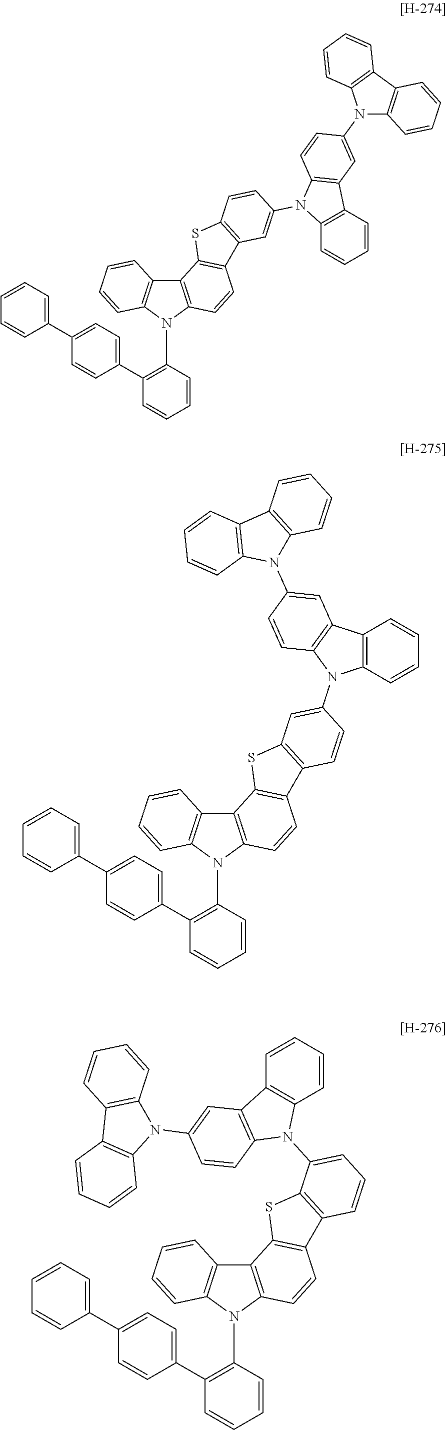 [SMILES: c1ccc(-c2ccc(-c3ccccc3-n3c4ccccc4c4c5sc6ccc(-n7c8ccccc8c8cc(-n9c%10ccccc%10c%10ccccc%109)ccc87)cc6c5ccc43)cc2)cc1 ; c1ccc(-c2ccc(-c3ccccc3-n3c4ccccc4c4c5sc6cc(-n7c8ccccc8c8cc(-n9c%10ccccc%10c%10ccccc%109)ccc87)ccc6c5ccc43)cc2)cc1 ; c1ccc(-c2ccc(-c3ccccc3-n3c4ccccc4c4c5sc6c(-n7c8ccccc8c8cc(-n9c%10ccccc%10c%10ccccc%109)ccc87)cccc6c5ccc43)cc2)cc1]

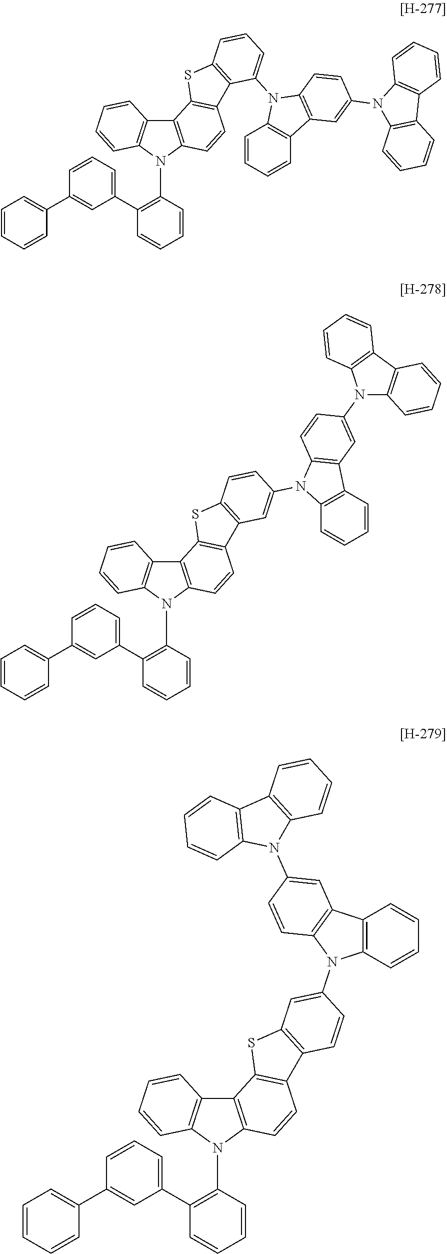 [SMILES: c1ccc(-c2cccc(-c3ccccc3-n3c4ccccc4c4c5sc6cccc(-n7c8ccccc8c8cc(-n9c%10ccccc%10c%10ccccc%109)ccc87)c6c5ccc43)c2)cc1 ; c1ccc(-c2cccc(-c3ccccc3-n3c4ccccc4c4c5sc6ccc(-n7c8ccccc8c8cc(-n9c%10ccccc%10c%10ccccc%109)ccc87)cc6c5ccc43)c2)cc1 ; c1ccc(-c2cccc(-c3ccccc3-n3c4ccccc4c4c5sc6cc(-n7c8ccccc8c8cc(-n9c%10ccccc%10c%10ccccc%109)ccc87)ccc6c5ccc43)c2)cc1]

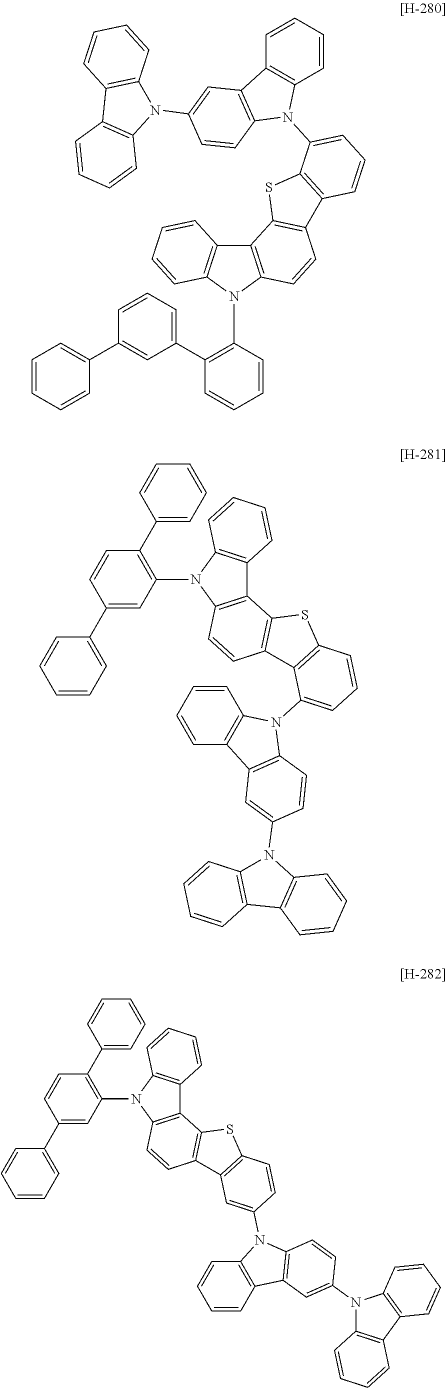 [SMILES: c1ccc(-c2cccc(-c3ccccc3-n3c4ccccc4c4c5sc6c(-n7c8ccccc8c8cc(-n9c%10ccccc%10c%10ccccc%109)ccc87)cccc6c5ccc43)c2)cc1 ; c1ccc(-c2ccc(-c3ccccc3)c(-n3c4ccccc4c4c5sc6cccc(-n7c8ccccc8c8cc(-n9c%10ccccc%10c%10ccccc%109)ccc87)c6c5ccc43)c2)cc1 ; c1ccc(-c2ccc(-c3ccccc3)c(-n3c4ccccc4c4c5sc6ccc(-n7c8ccccc8c8cc(-n9c%10ccccc%10c%10ccccc%109)ccc87)cc6c5ccc43)c2)cc1]

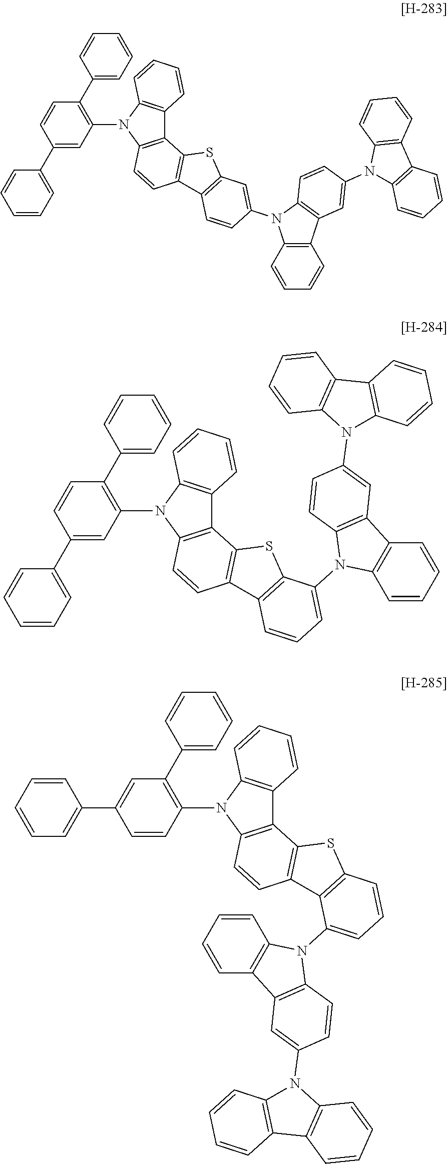 [SMILES: c1ccc(-c2ccc(-c3ccccc3)c(-n3c4ccccc4c4c5sc6cc(-n7c8ccccc8c8cc(-n9c%10ccccc%10c%10ccccc%109)ccc87)ccc6c5ccc43)c2)cc1 ; c1ccc(-c2ccc(-c3ccccc3)c(-n3c4ccccc4c4c5sc6c(-n7c8ccccc8c8cc(-n9c%10ccccc%10c%10ccccc%109)ccc87)cccc6c5ccc43)c2)cc1 ; c1ccc(-c2ccc(-n3c4ccccc4c4c5sc6cccc(-n7c8ccccc8c8cc(-n9c%10ccccc%10c%10ccccc%109)ccc87)c6c5ccc43)c(-c3ccccc3)c2)cc1]

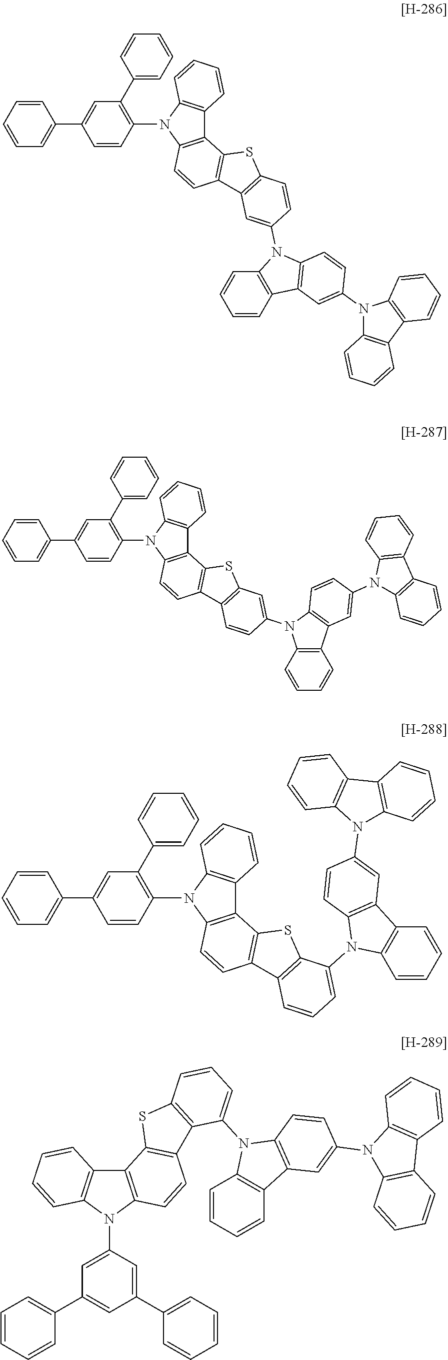 [SMILES: c1ccc(-c2ccc(-n3c4ccccc4c4c5sc6ccc(-n7c8ccccc8c8cc(-n9c%10ccccc%10c%10ccccc%109)ccc87)cc6c5ccc43)c(-c3ccccc3)c2)cc1 ; c1ccc(-c2ccc(-n3c4ccccc4c4c5sc6cc(-n7c8ccccc8c8cc(-n9c%10ccccc%10c%10ccccc%109)ccc87)ccc6c5ccc43)c(-c3ccccc3)c2)cc1 ; c1ccc(-c2ccc(-n3c4ccccc4c4c5sc6c(-n7c8ccccc8c8cc(-n9c%10ccccc%10c%10ccccc%109)ccc87)cccc6c5ccc43)c(-c3ccccc3)c2)cc1 ; c1ccc(-c2cc(-c3ccccc3)cc(-n3c4ccccc4c4c5sc6cccc(-n7c8ccccc8c8cc(-n9c%10ccccc%10c%10ccccc%109)ccc87)c6c5ccc43)c2)cc1]

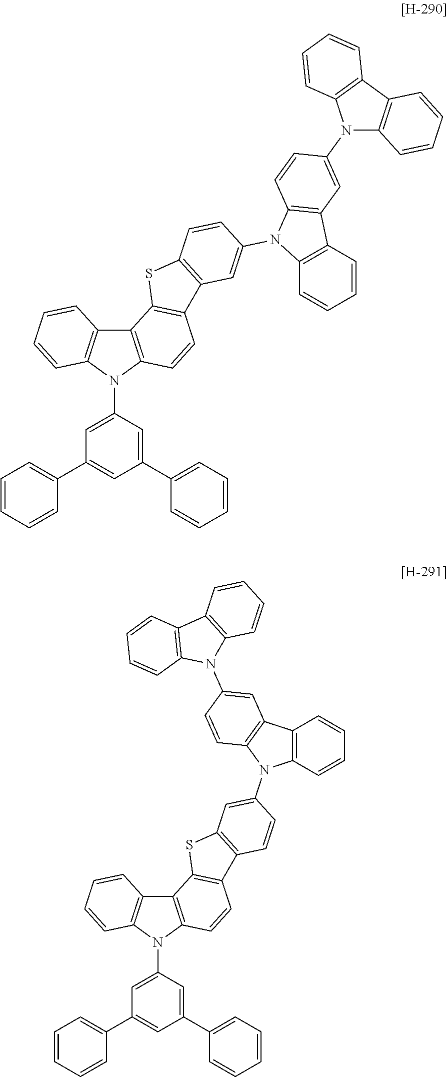 [SMILES: c1ccc(-c2cc(-c3ccccc3)cc(-n3c4ccccc4c4c5sc6ccc(-n7c8ccccc8c8cc(-n9c%10ccccc%10c%10ccccc%109)ccc87)cc6c5ccc43)c2)cc1 ; c1ccc(-c2cc(-c3ccccc3)cc(-n3c4ccccc4c4c5sc6cc(-n7c8ccccc8c8cc(-n9c%10ccccc%10c%10ccccc%109)ccc87)ccc6c5ccc43)c2)cc1]

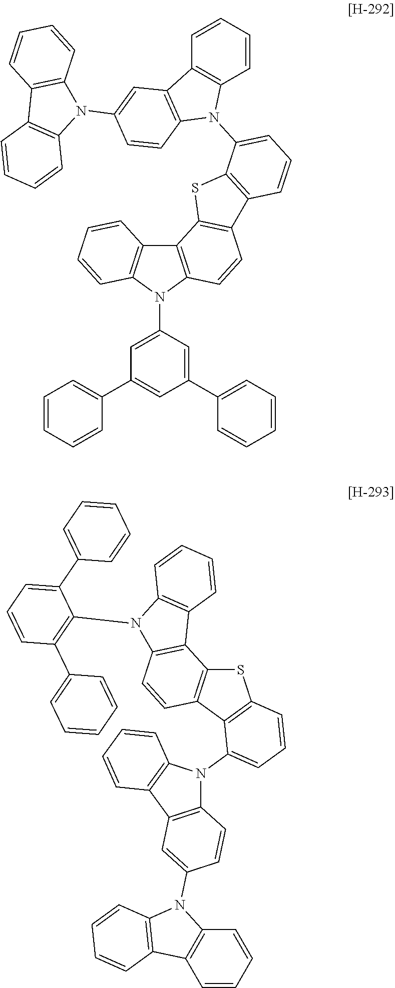 [SMILES: c1ccc(-c2cc(-c3ccccc3)cc(-n3c4ccccc4c4c5sc6c(-n7c8ccccc8c8cc(-n9c%10ccccc%10c%10ccccc%109)ccc87)cccc6c5ccc43)c2)cc1 ; c1ccc(-c2cccc(-c3ccccc3)c2-n2c3ccccc3c3c4sc5cccc(-n6c7ccccc7c7cc(-n8c9ccccc9c9ccccc98)ccc76)c5c4ccc32)cc1]

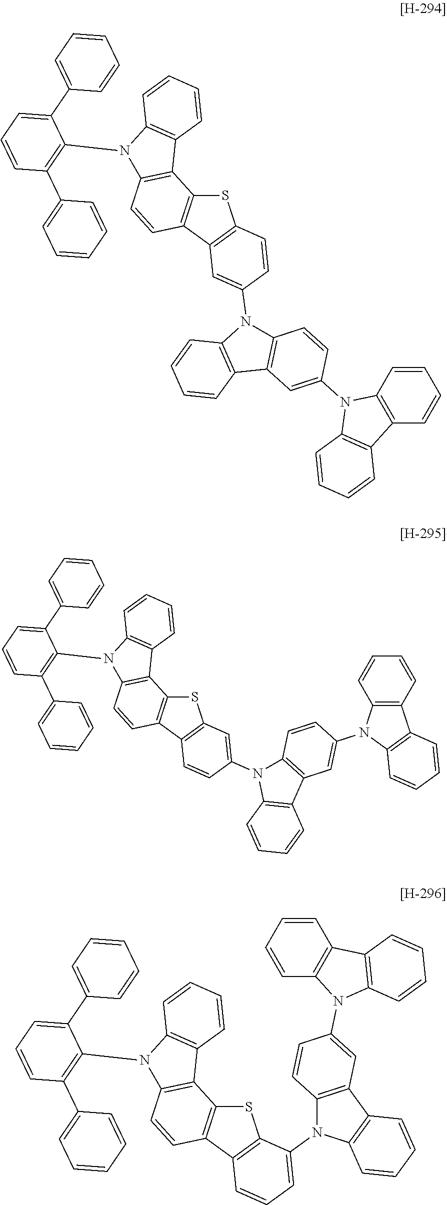 [SMILES: c1ccc(-c2cccc(-c3ccccc3)c2-n2c3ccccc3c3c4sc5ccc(-n6c7ccccc7c7cc(-n8c9ccccc9c9ccccc98)ccc76)cc5c4ccc32)cc1 ; c1ccc(-c2cccc(-c3ccccc3)c2-n2c3ccccc3c3c4sc5cc(-n6c7ccccc7c7cc(-n8c9ccccc9c9ccccc98)ccc76)ccc5c4ccc32)cc1 ; c1ccc(-c2cccc(-c3ccccc3)c2-n2c3ccccc3c3c4sc5c(-n6c7ccccc7c7cc(-n8c9ccccc9c9ccccc98)ccc76)cccc5c4ccc32)cc1]

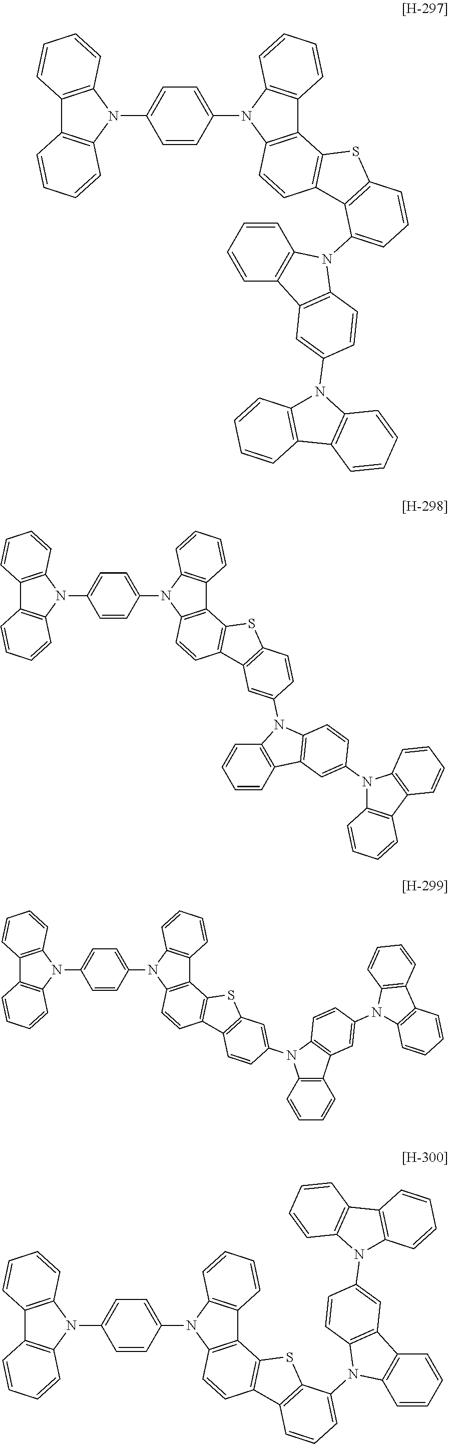 [SMILES: c1cc(-n2c3ccccc3c3cc(-n4c5ccccc5c5ccccc54)ccc32)c2c(c1)sc1c2ccc2c1c1ccccc1n2-c1ccc(-n2c3ccccc3c3ccccc32)cc1 ; c1ccc2c(c1)c1ccccc1n2-c1ccc(-n2c3ccccc3c3c4sc5ccc(-n6c7ccccc7c7cc(-n8c9ccccc9c9ccccc98)ccc76)cc5c4ccc32)cc1 ; c1ccc2c(c1)c1ccccc1n2-c1ccc(-n2c3ccccc3c3c4sc5cc(-n6c7ccccc7c7cc(-n8c9ccccc9c9ccccc98)ccc76)ccc5c4ccc32)cc1 ; c1cc(-n2c3ccccc3c3cc(-n4c5ccccc5c5ccccc54)ccc32)c2sc3c(ccc4c3c3ccccc3n4-c3ccc(-n4c5ccccc5c5ccccc54)cc3)c2c1]

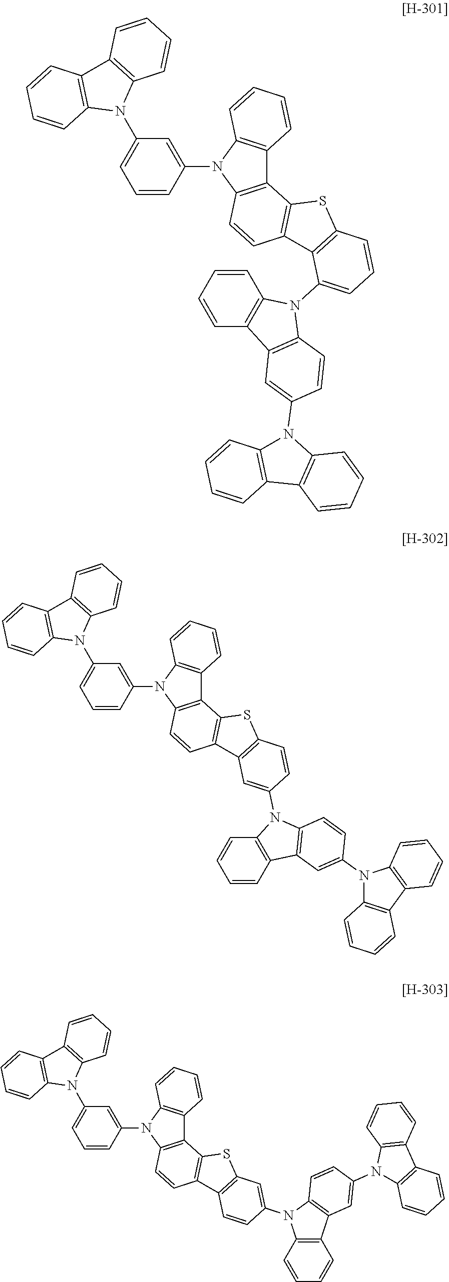 [SMILES: c1cc(-n2c3ccccc3c3ccccc32)cc(-n2c3ccccc3c3c4sc5cccc(-n6c7ccccc7c7cc(-n8c9ccccc9c9ccccc98)ccc76)c5c4ccc32)c1 ; c1cc(-n2c3ccccc3c3ccccc32)cc(-n2c3ccccc3c3c4sc5ccc(-n6c7ccccc7c7cc(-n8c9ccccc9c9ccccc98)ccc76)cc5c4ccc32)c1 ; c1cc(-n2c3ccccc3c3ccccc32)cc(-n2c3ccccc3c3c4sc5cc(-n6c7ccccc7c7cc(-n8c9ccccc9c9ccccc98)ccc76)ccc5c4ccc32)c1]

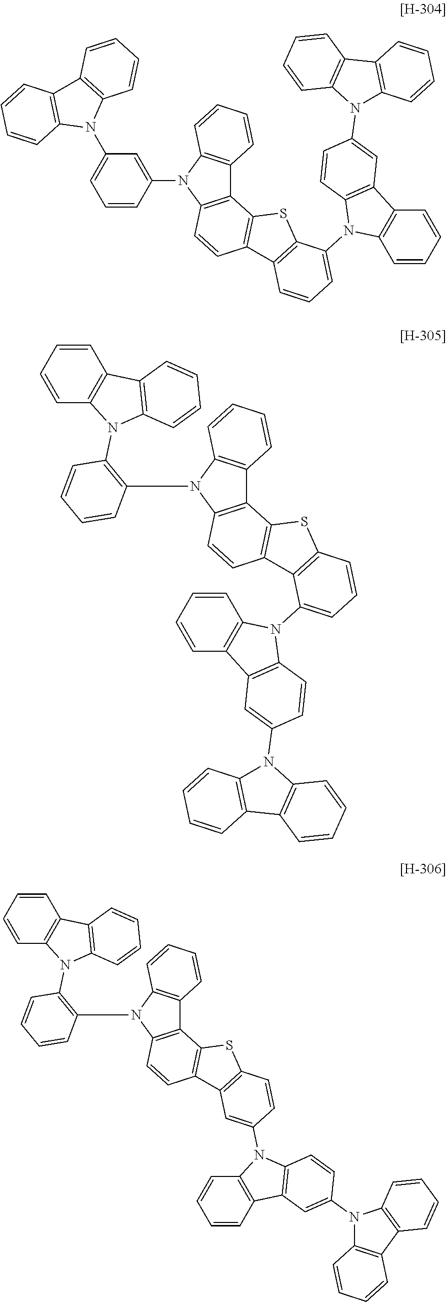 [SMILES: c1cc(-n2c3ccccc3c3ccccc32)cc(-n2c3ccccc3c3c4sc5c(-n6c7ccccc7c7cc(-n8c9ccccc9c9ccccc98)ccc76)cccc5c4ccc32)c1 ; c1ccc(-n2c3ccccc3c3c4sc5cccc(-n6c7ccccc7c7cc(-n8c9ccccc9c9ccccc98)ccc76)c5c4ccc32)c(-n2c3ccccc3c3ccccc32)c1 ; c1ccc(-n2c3ccccc3c3c4sc5ccc(-n6c7ccccc7c7cc(-n8c9ccccc9c9ccccc98)ccc76)cc5c4ccc32)c(-n2c3ccccc3c3ccccc32)c1]

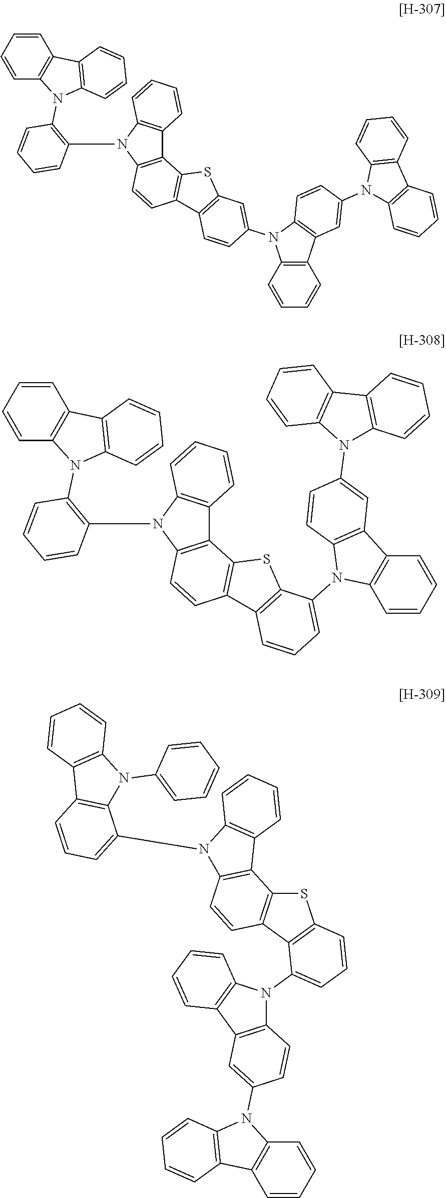 [SMILES: c1ccc(-n2c3ccccc3c3c4sc5cc(-n6c7ccccc7c7cc(-n8c9ccccc9c9ccccc98)ccc76)ccc5c4ccc32)c(-n2c3ccccc3c3ccccc32)c1 ; c1ccc(-n2c3ccccc3c3c4sc5c(-n6c7ccccc7c7cc(-n8c9ccccc9c9ccccc98)ccc76)cccc5c4ccc32)c(-n2c3ccccc3c3ccccc32)c1 ; c1ccc(-n2c3ccccc3c3cccc(-n4c5ccccc5c5c6sc7cccc(-n8c9ccccc9c9cc(-n%10c%11ccccc%11c%11ccccc%11%10)ccc98)c7c6ccc54)c32)cc1]

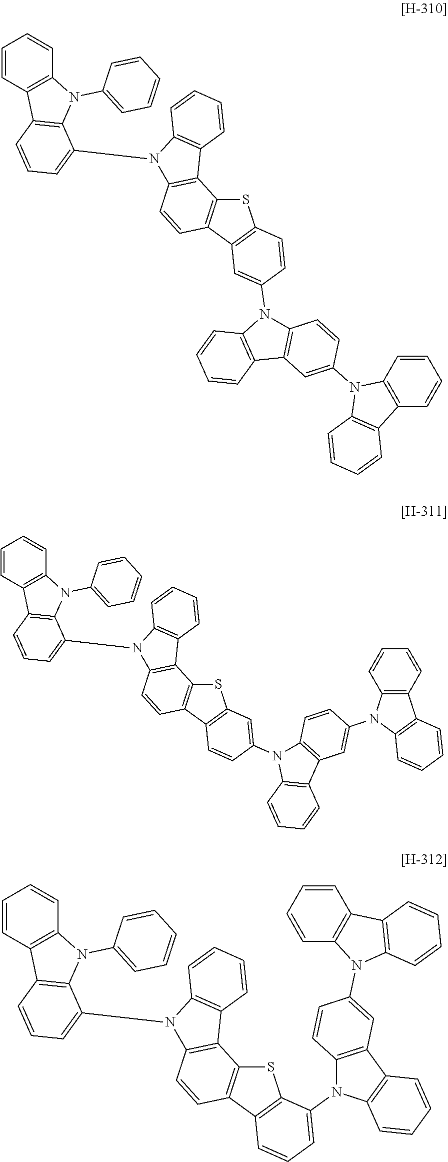 [SMILES: c1ccc(-n2c3ccccc3c3cccc(-n4c5ccccc5c5c6sc7ccc(-n8c9ccccc9c9cc(-n%10c%11ccccc%11c%11ccccc%11%10)ccc98)cc7c6ccc54)c32)cc1 ; c1ccc(-n2c3ccccc3c3cccc(-n4c5ccccc5c5c6sc7cc(-n8c9ccccc9c9cc(-n%10c%11ccccc%11c%11ccccc%11%10)ccc98)ccc7c6ccc54)c32)cc1 ; c1ccc(-n2c3ccccc3c3cccc(-n4c5ccccc5c5c6sc7c(-n8c9ccccc9c9cc(-n%10c%11ccccc%11c%11ccccc%11%10)ccc98)cccc7c6ccc54)c32)cc1]

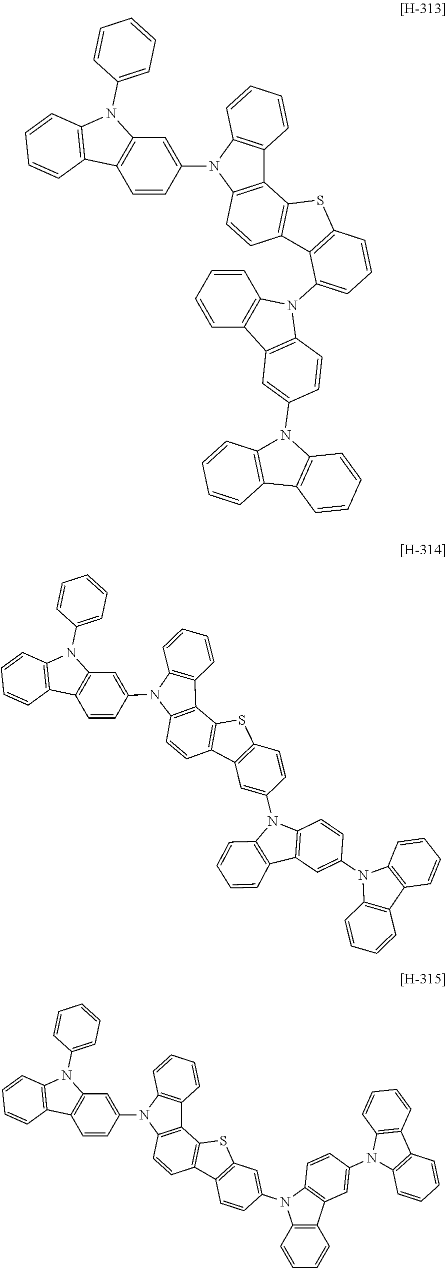 [SMILES: c1ccc(-n2c3ccccc3c3ccc(-n4c5ccccc5c5c6sc7cccc(-n8c9ccccc9c9cc(-n%10c%11ccccc%11c%11ccccc%11%10)ccc98)c7c6ccc54)cc32)cc1 ; c1ccc(-n2c3ccccc3c3ccc(-n4c5ccccc5c5c6sc7ccc(-n8c9ccccc9c9cc(-n%10c%11ccccc%11c%11ccccc%11%10)ccc98)cc7c6ccc54)cc32)cc1 ; c1ccc(-n2c3ccccc3c3ccc(-n4c5ccccc5c5c6sc7cc(-n8c9ccccc9c9cc(-n%10c%11ccccc%11c%11ccccc%11%10)ccc98)ccc7c6ccc54)cc32)cc1]

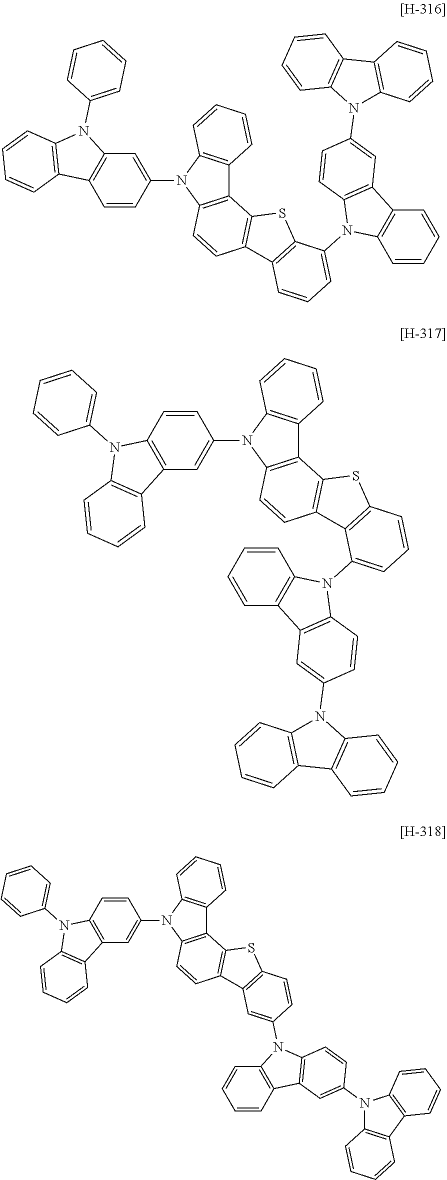 [SMILES: c1ccc(-n2c3ccccc3c3ccc(-n4c5ccccc5c5c6sc7c(-n8c9ccccc9c9cc(-n%10c%11ccccc%11c%11ccccc%11%10)ccc98)cccc7c6ccc54)cc32)cc1 ; c1ccc(-n2c3ccccc3c3cc(-n4c5ccccc5c5c6sc7cccc(-n8c9ccccc9c9cc(-n%10c%11ccccc%11c%11ccccc%11%10)ccc98)c7c6ccc54)ccc32)cc1 ; c1ccc(-n2c3ccccc3c3cc(-n4c5ccccc5c5c6sc7ccc(-n8c9ccccc9c9cc(-n%10c%11ccccc%11c%11ccccc%11%10)ccc98)cc7c6ccc54)ccc32)cc1]

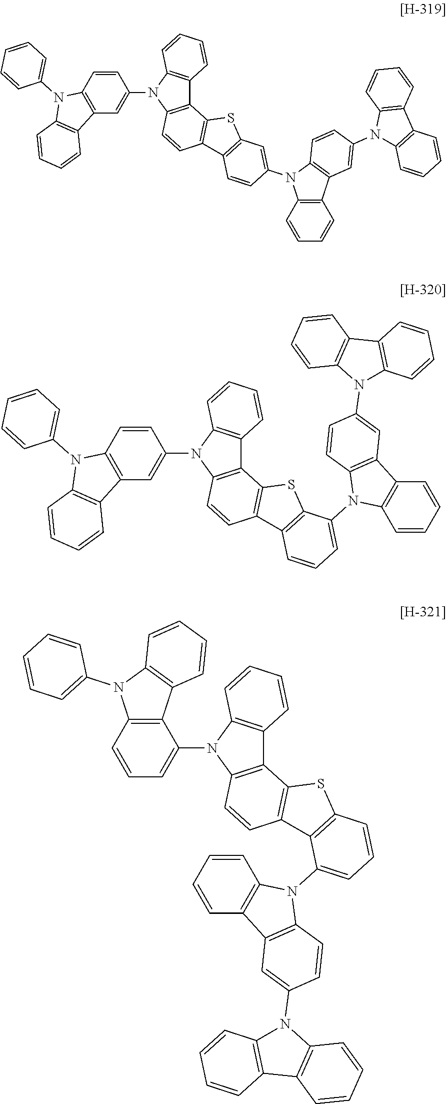 [SMILES: c1ccc(-n2c3ccccc3c3cc(-n4c5ccccc5c5c6sc7cc(-n8c9ccccc9c9cc(-n%10c%11ccccc%11c%11ccccc%11%10)ccc98)ccc7c6ccc54)ccc32)cc1 ; c1ccc(-n2c3ccccc3c3cc(-n4c5ccccc5c5c6sc7c(-n8c9ccccc9c9cc(-n%10c%11ccccc%11c%11ccccc%11%10)ccc98)cccc7c6ccc54)ccc32)cc1 ; c1ccc(-n2c3ccccc3c3c(-n4c5ccccc5c5c6sc7cccc(-n8c9ccccc9c9cc(-n%10c%11ccccc%11c%11ccccc%11%10)ccc98)c7c6ccc54)cccc32)cc1]

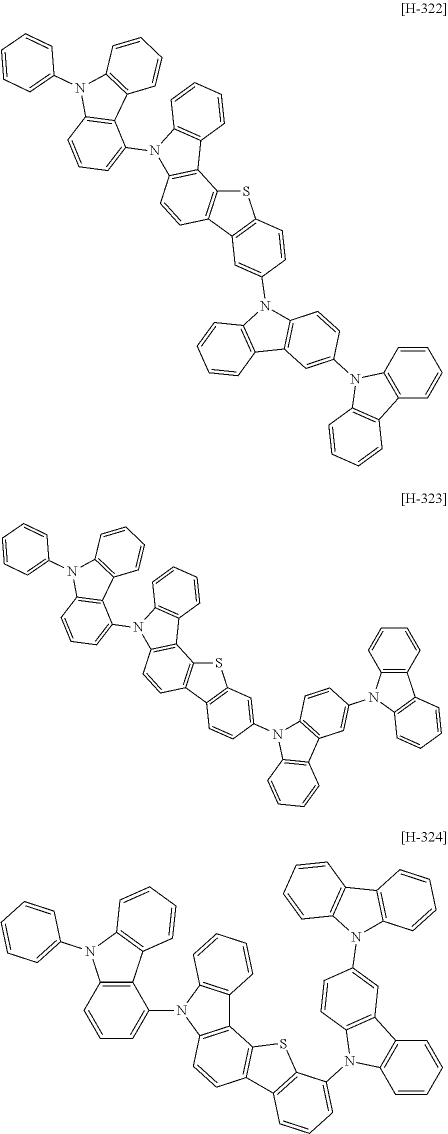 [SMILES: c1ccc(-n2c3ccccc3c3c(-n4c5ccccc5c5c6sc7ccc(-n8c9ccccc9c9cc(-n%10c%11ccccc%11c%11ccccc%11%10)ccc98)cc7c6ccc54)cccc32)cc1 ; c1ccc(-n2c3ccccc3c3c(-n4c5ccccc5c5c6sc7cc(-n8c9ccccc9c9cc(-n%10c%11ccccc%11c%11ccccc%11%10)ccc98)ccc7c6ccc54)cccc32)cc1 ; c1ccc(-n2c3ccccc3c3c(-n4c5ccccc5c5c6sc7c(-n8c9ccccc9c9cc(-n%10c%11ccccc%11c%11ccccc%11%10)ccc98)cccc7c6ccc54)cccc32)cc1]

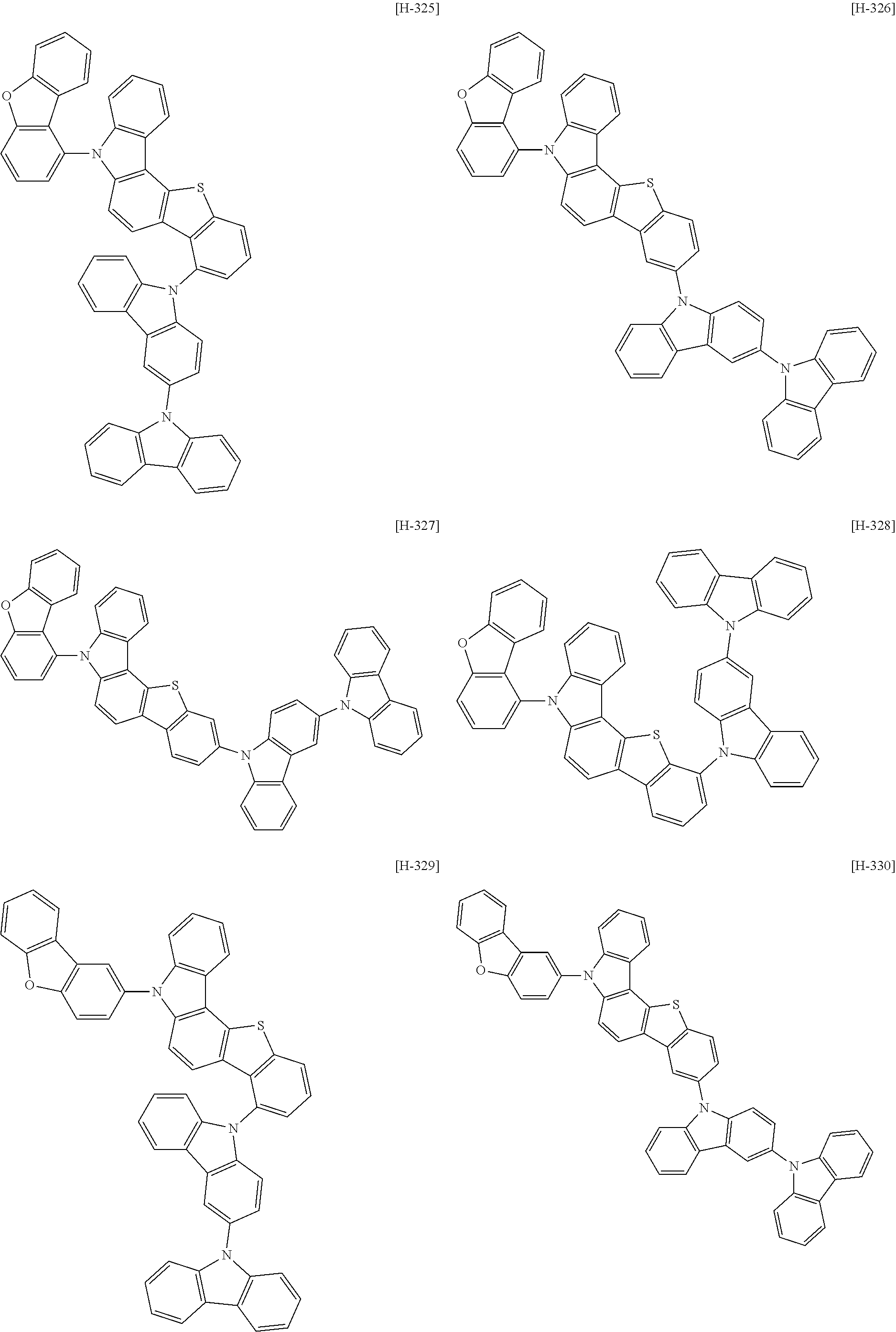

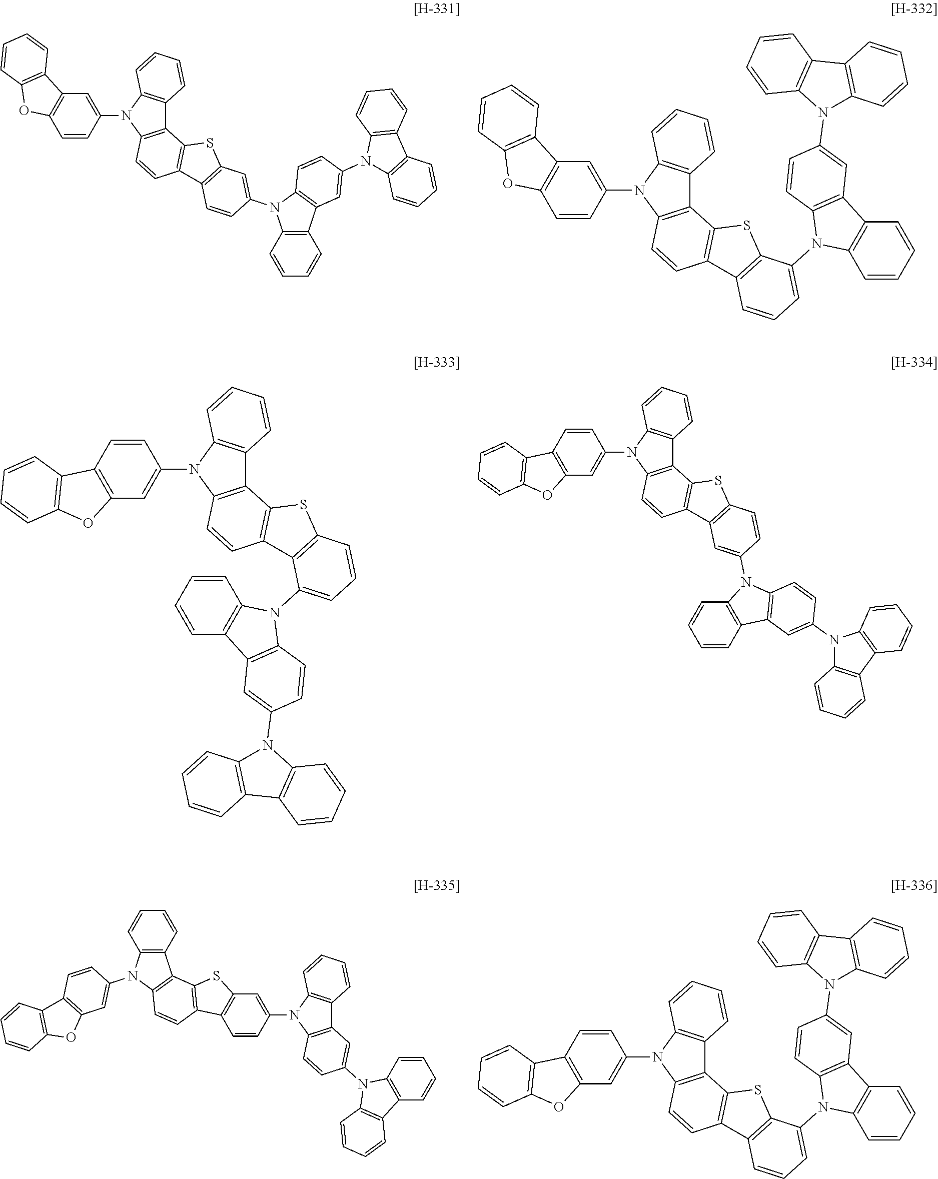

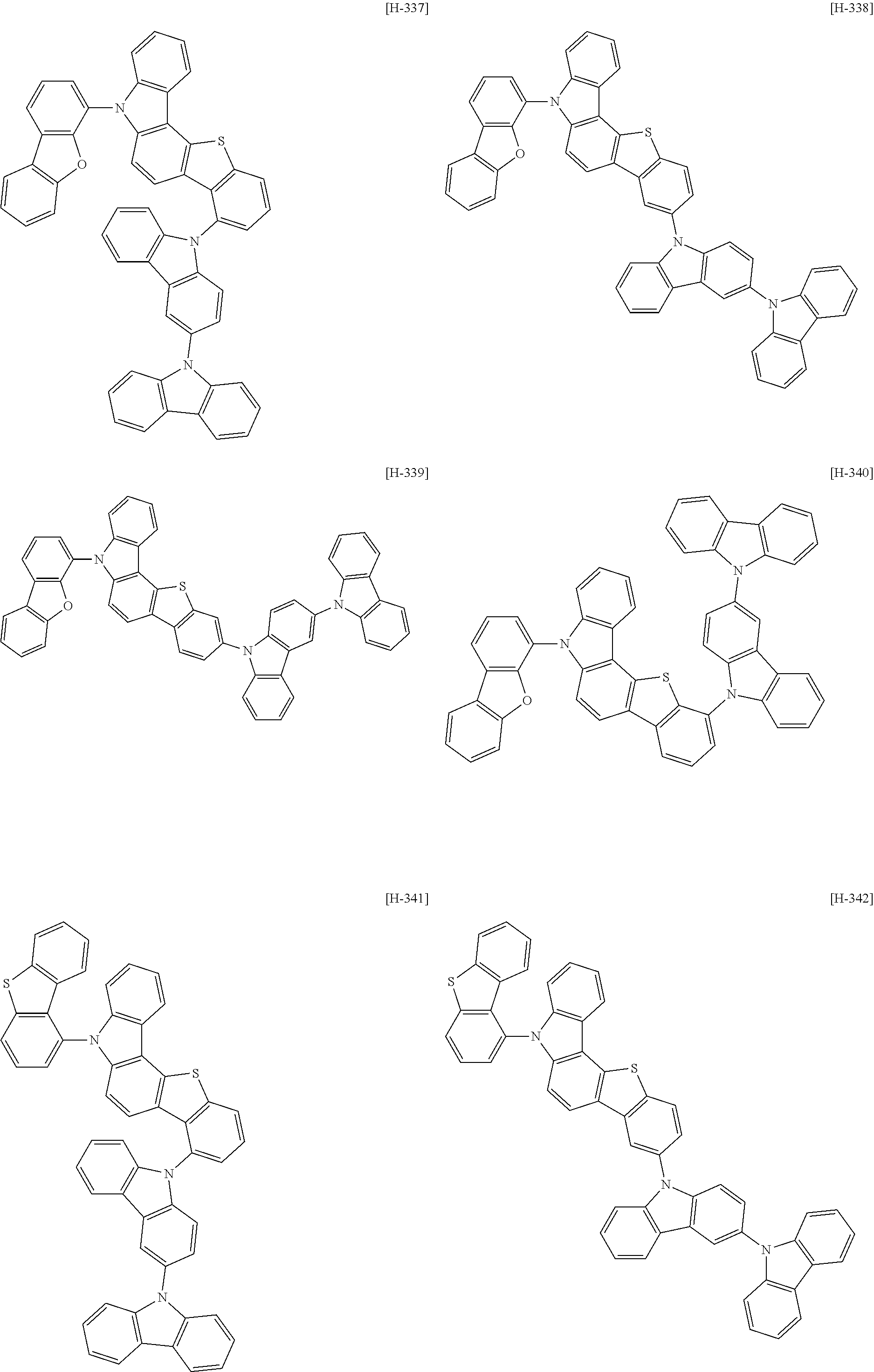

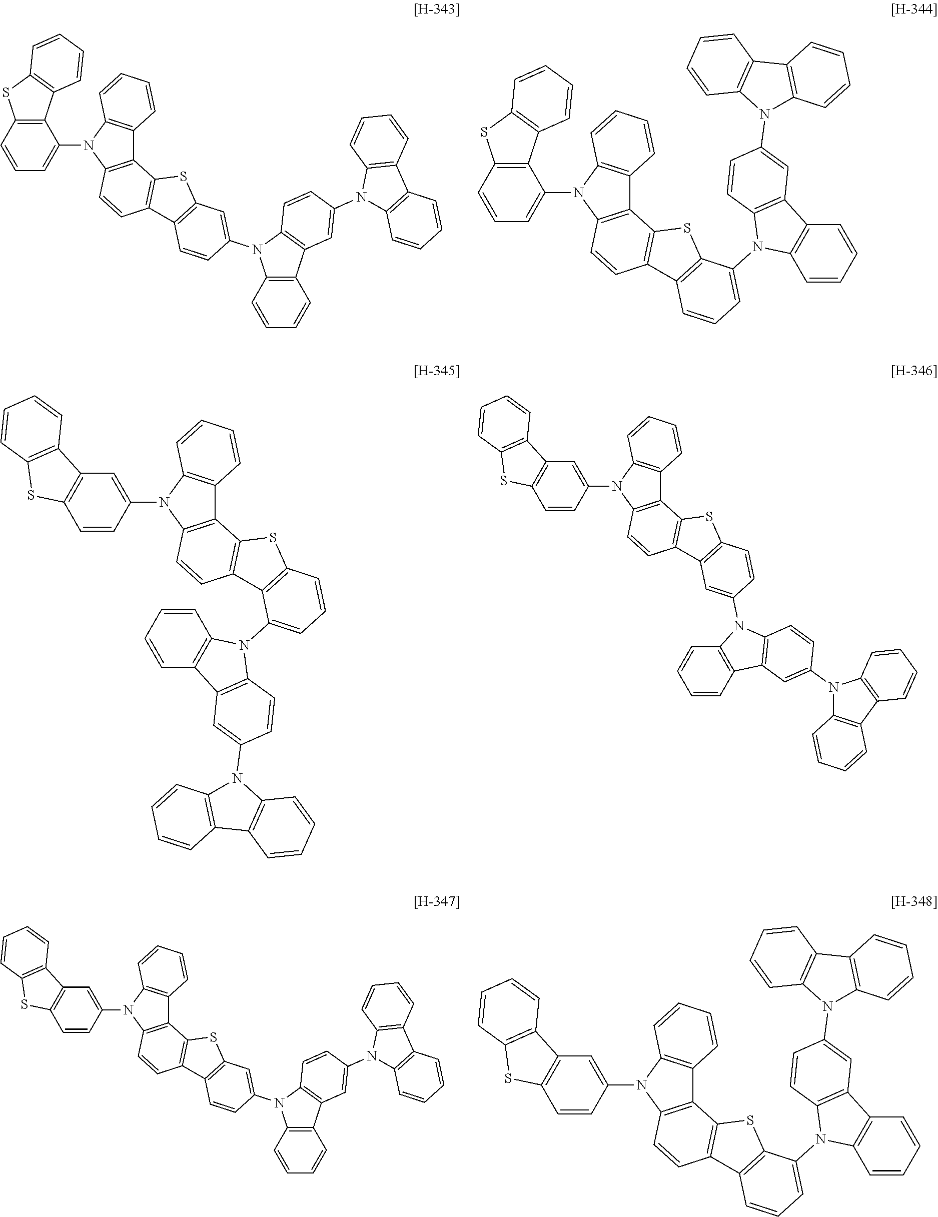

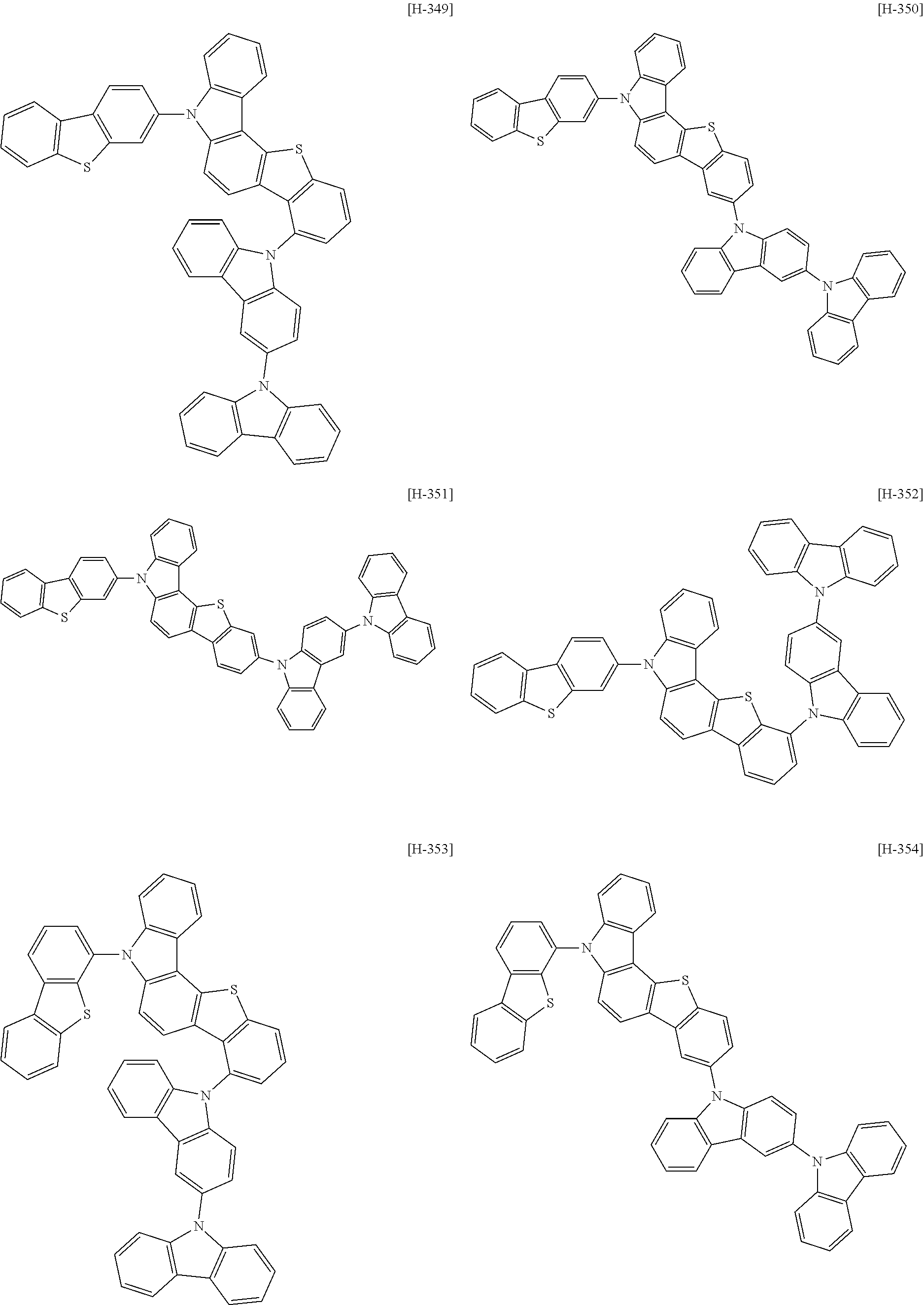

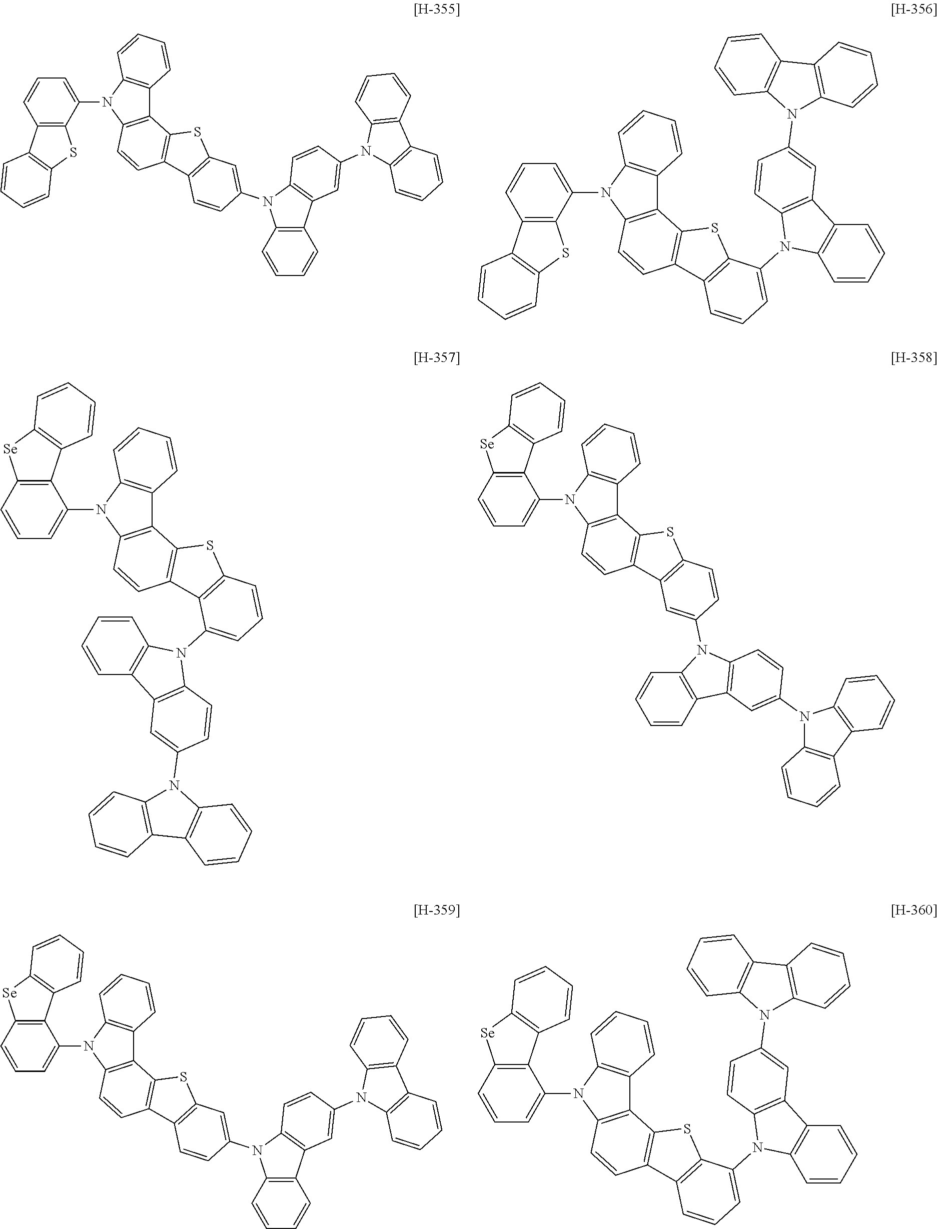

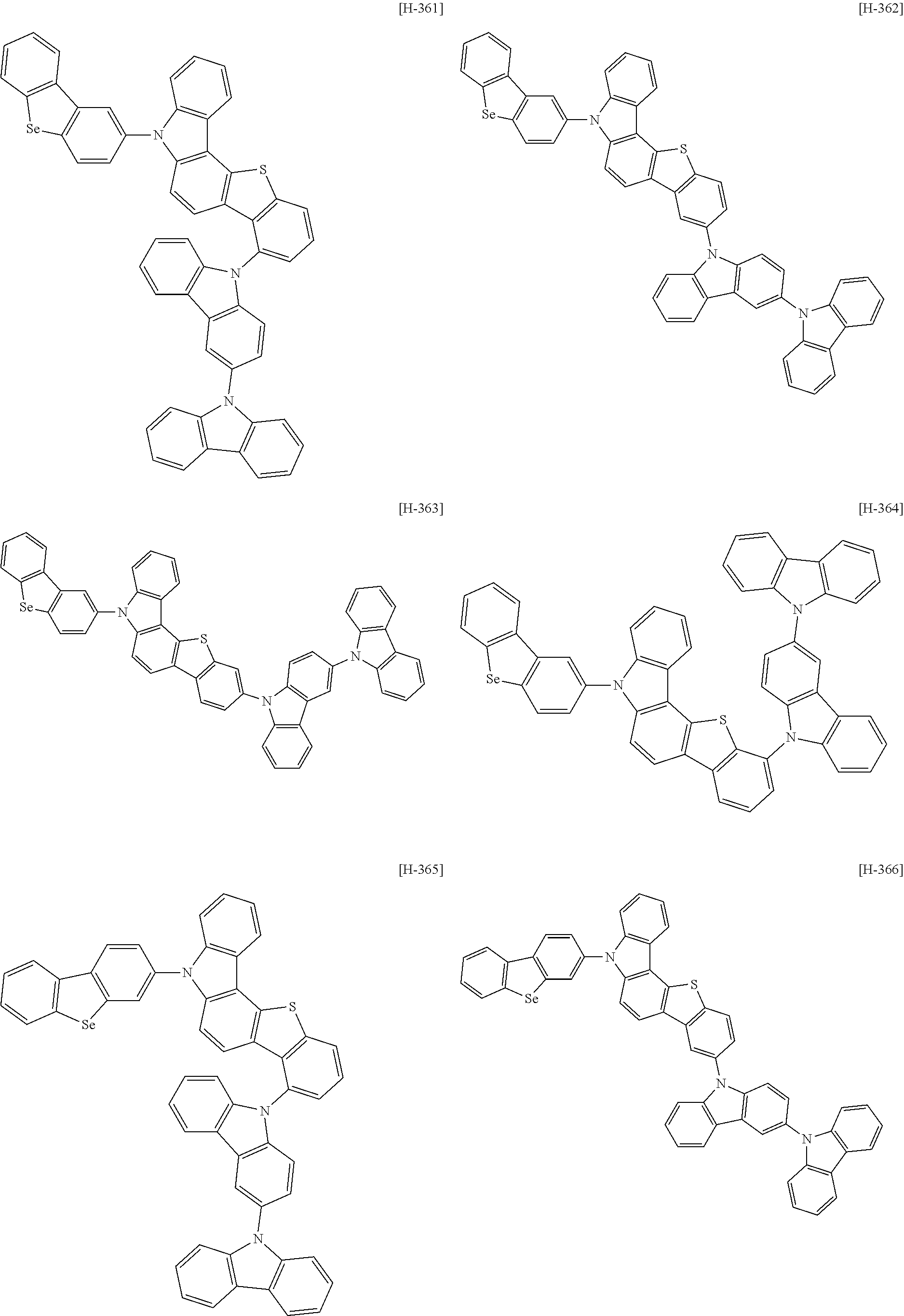

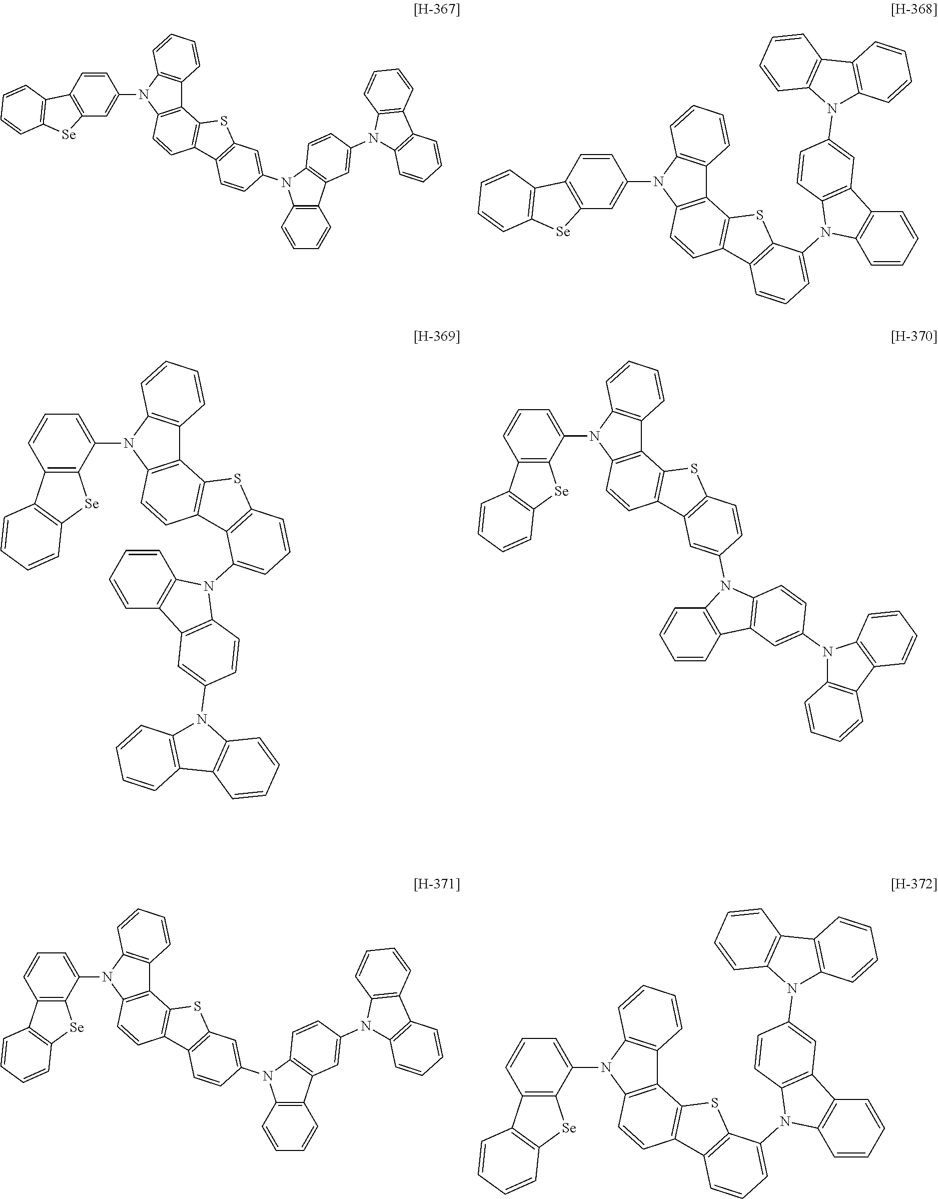

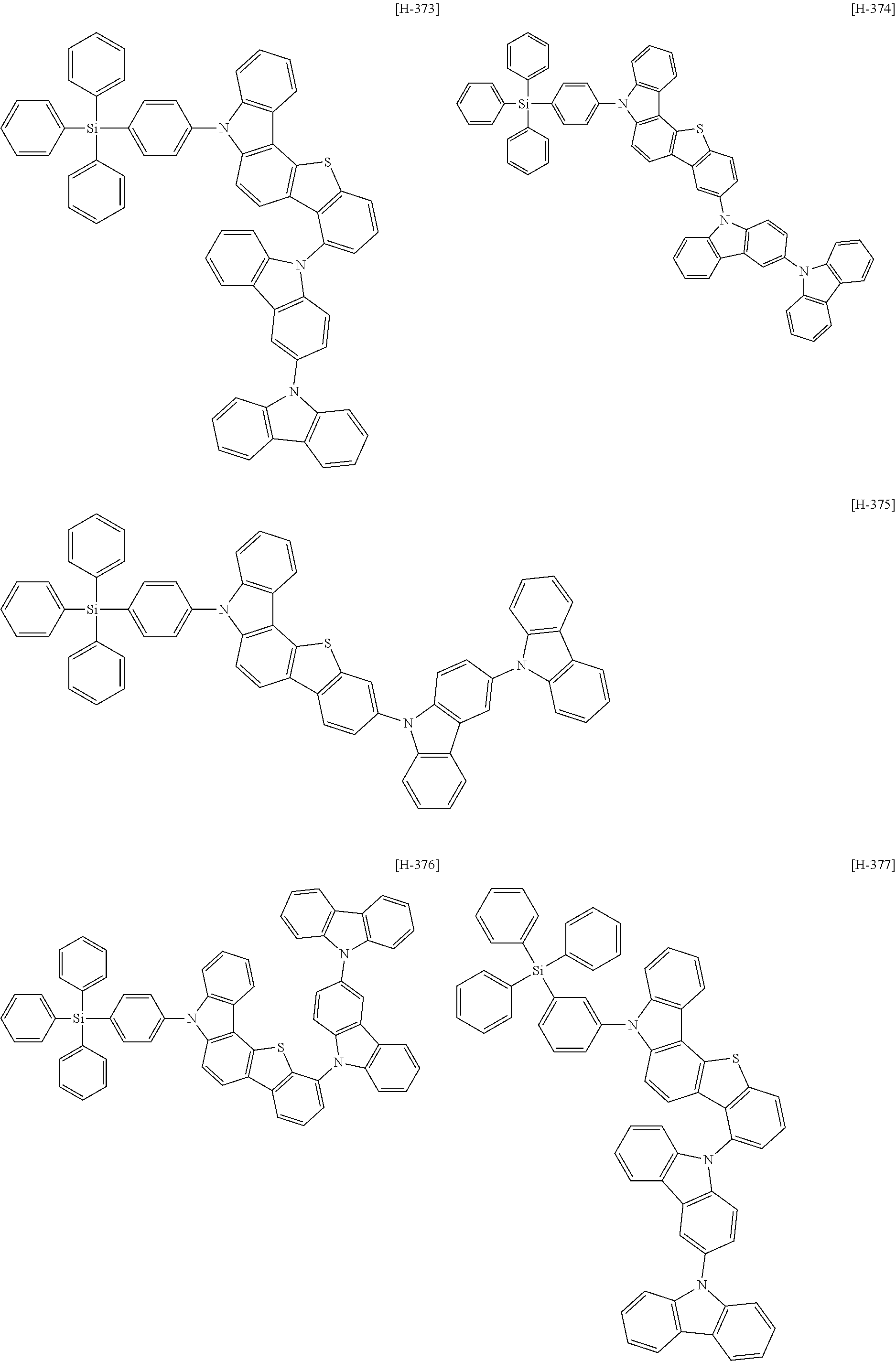

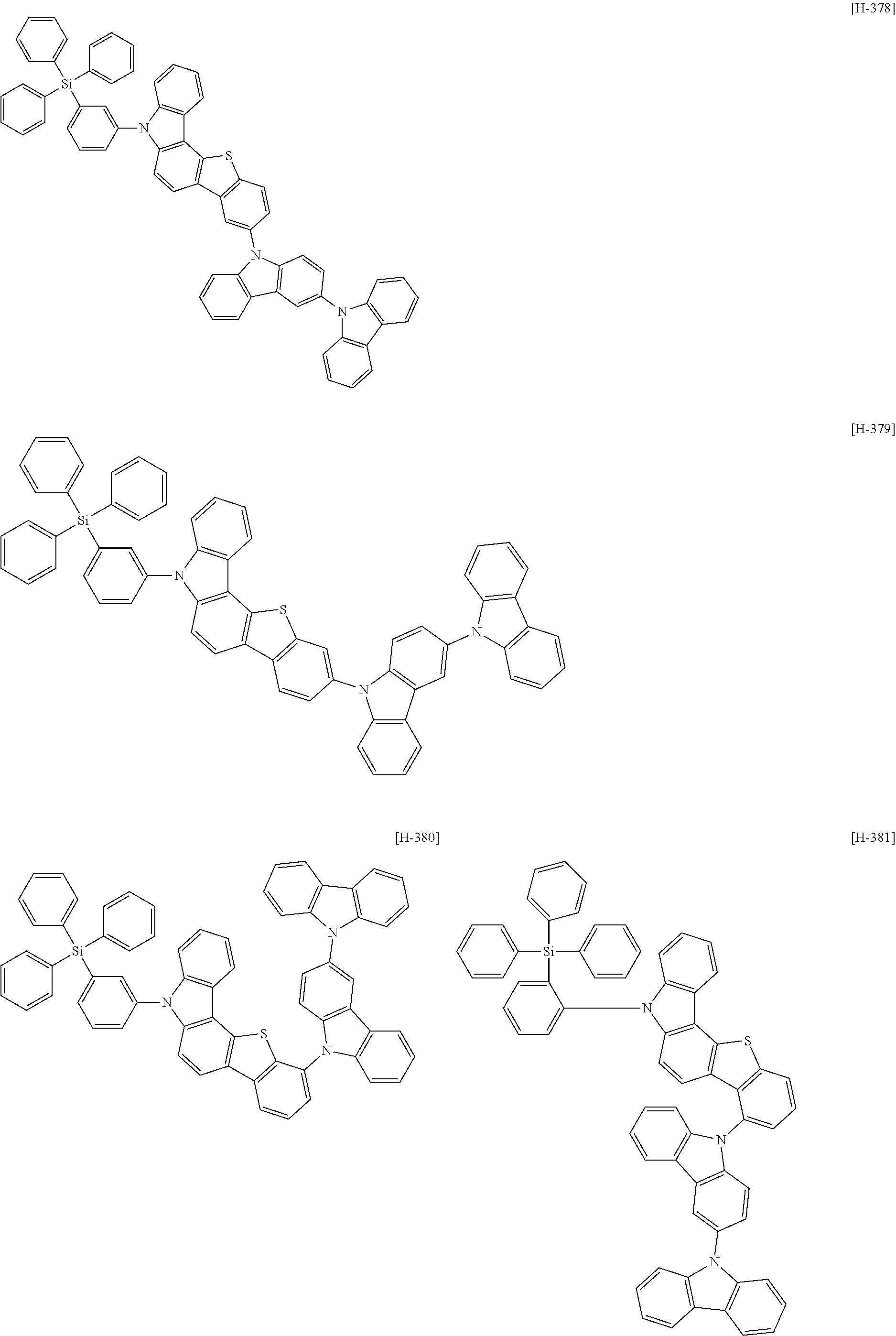

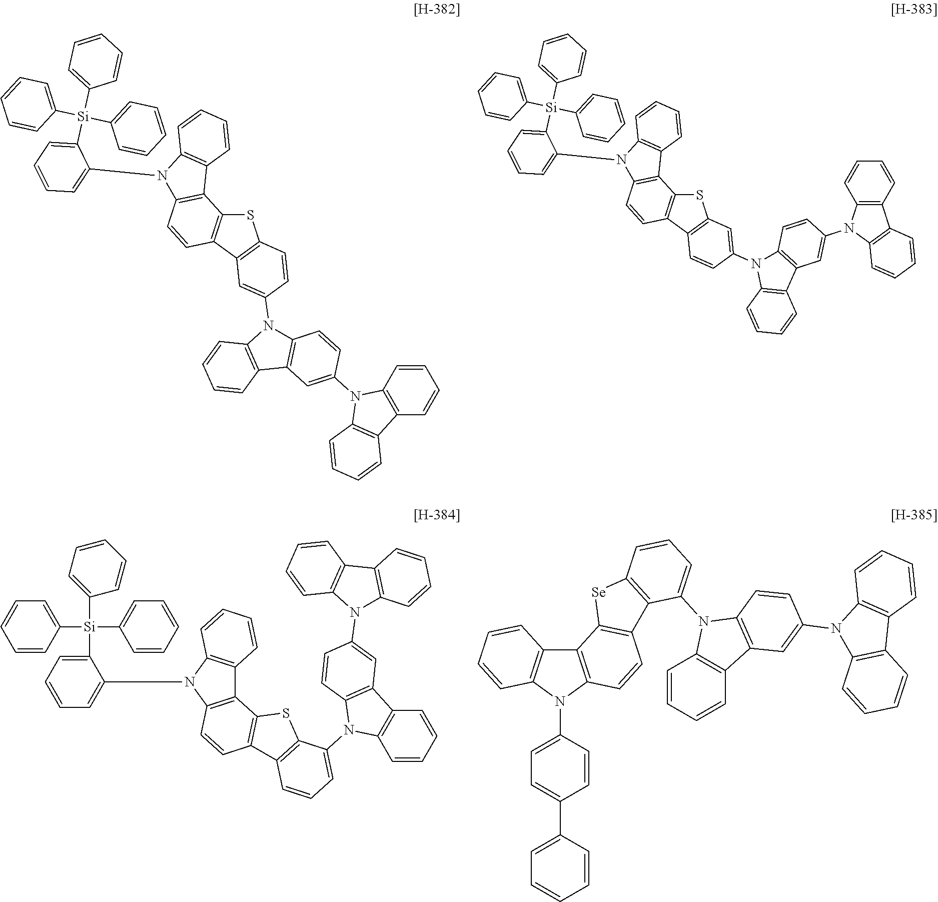 [SMILES: c1ccc([Si](c2ccccc2)(c2ccccc2)c2ccccc2-n2c3ccccc3c3c4sc5ccc(-n6c7ccccc7c7cc(-n8c9ccccc9c9ccccc98)ccc76)cc5c4ccc32)cc1 ; c1ccc([Si](c2ccccc2)(c2ccccc2)c2ccccc2-n2c3ccccc3c3c4sc5cc(-n6c7ccccc7c7cc(-n8c9ccccc9c9ccccc98)ccc76)ccc5c4ccc32)cc1 ; c1ccc([Si](c2ccccc2)(c2ccccc2)c2ccccc2-n2c3ccccc3c3c4sc5c(-n6c7ccccc7c7cc(-n8c9ccccc9c9ccccc98)ccc76)cccc5c4ccc32)cc1 ; c1ccc(-c2ccc(-n3c4ccccc4c4c5[se]c6cccc(-n7c8ccccc8c8cc(-n9c%10ccccc%10c%10ccccc%109)ccc87)c6c5ccc43)cc2)cc1]

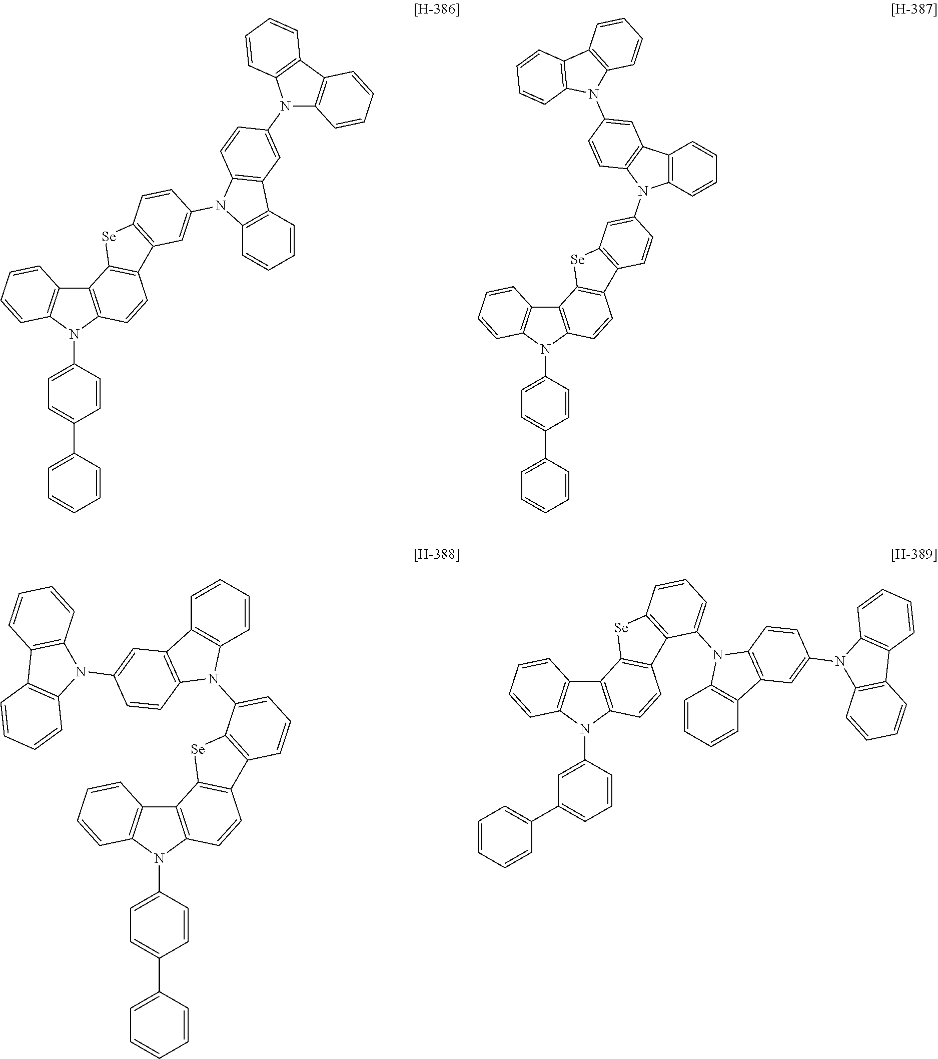 [SMILES: c1ccc(-c2ccc(-n3c4ccccc4c4c5[se]c6ccc(-n7c8ccccc8c8cc(-n9c%10ccccc%10c%10ccccc%109)ccc87)cc6c5ccc43)cc2)cc1 ; c1ccc(-c2ccc(-n3c4ccccc4c4c5[se]c6cc(-n7c8ccccc8c8cc(-n9c%10ccccc%10c%10ccccc%109)ccc87)ccc6c5ccc43)cc2)cc1 ; c1ccc(-c2ccc(-n3c4ccccc4c4c5[se]c6c(-n7c8ccccc8c8cc(-n9c%10ccccc%10c%10ccccc%109)ccc87)cccc6c5ccc43)cc2)cc1 ; c1ccc(-c2cccc(-n3c4ccccc4c4c5[se]c6cccc(-n7c8ccccc8c8cc(-n9c%10ccccc%10c%10ccccc%109)ccc87)c6c5ccc43)c2)cc1]

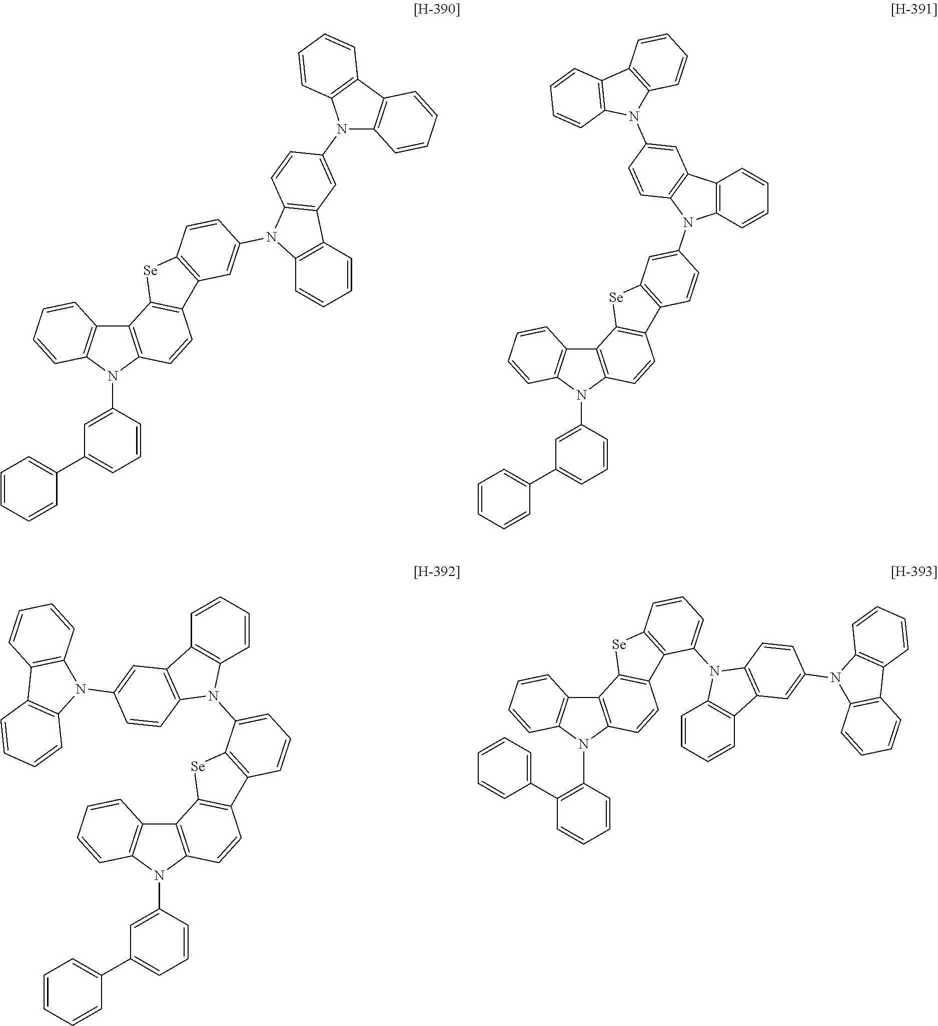 [SMILES: c1ccc(-c2cccc(-n3c4ccccc4c4c5[se]c6ccc(-n7c8ccccc8c8cc(-n9c%10ccccc%10c%10ccccc%109)ccc87)cc6c5ccc43)c2)cc1 ; c1ccc(-c2cccc(-n3c4ccccc4c4c5[se]c6cc(-n7c8ccccc8c8cc(-n9c%10ccccc%10c%10ccccc%109)ccc87)ccc6c5ccc43)c2)cc1 ; c1ccc(-c2cccc(-n3c4ccccc4c4c5[se]c6c(-n7c8ccccc8c8cc(-n9c%10ccccc%10c%10ccccc%109)ccc87)cccc6c5ccc43)c2)cc1 ; c1ccc(-c2ccccc2-n2c3ccccc3c3c4[se]c5cccc(-n6c7ccccc7c7cc(-n8c9ccccc9c9ccccc98)ccc76)c5c4ccc32)cc1]

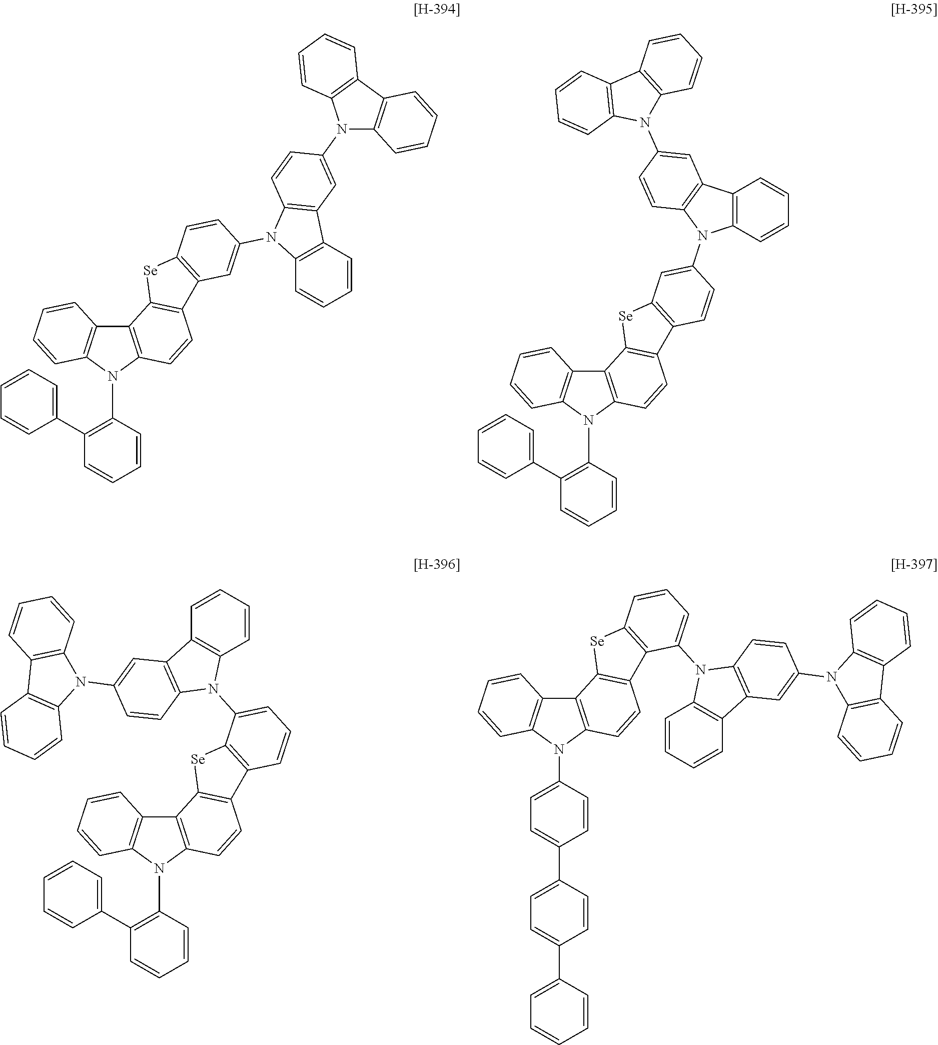 [SMILES: c1ccc(-c2ccccc2-n2c3ccccc3c3c4[se]c5ccc(-n6c7ccccc7c7cc(-n8c9ccccc9c9ccccc98)ccc76)cc5c4ccc32)cc1 ; c1ccc(-c2ccccc2-n2c3ccccc3c3c4[se]c5cc(-n6c7ccccc7c7cc(-n8c9ccccc9c9ccccc98)ccc76)ccc5c4ccc32)cc1 ; c1ccc(-c2ccccc2-n2c3ccccc3c3c4[se]c5c(-n6c7ccccc7c7cc(-n8c9ccccc9c9ccccc98)ccc76)cccc5c4ccc32)cc1 ; c1ccc(-c2ccc(-c3ccc(-n4c5ccccc5c5c6[se]c7cccc(-n8c9ccccc9c9cc(-n%10c%11ccccc%11c%11ccccc%11%10)ccc98)c7c6ccc54)cc3)cc2)cc1]

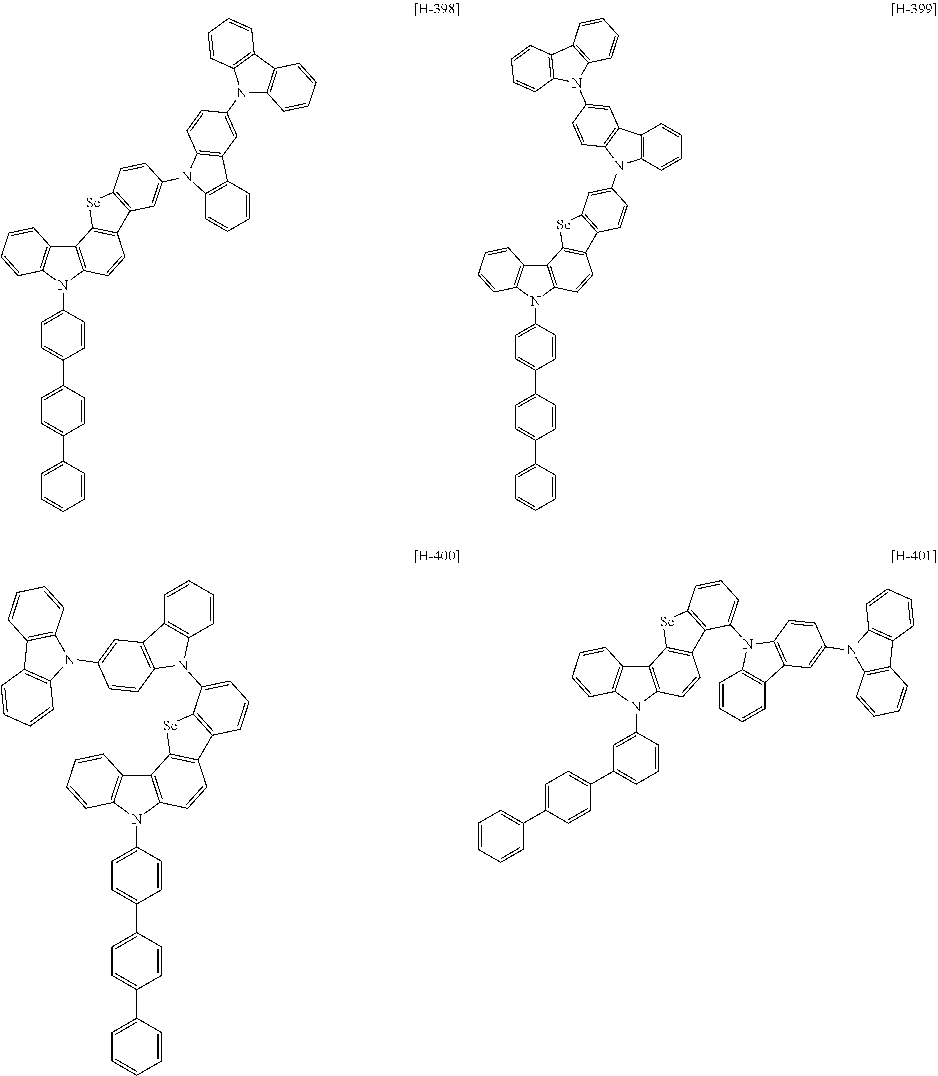 [SMILES: c1ccc(-c2ccc(-c3ccc(-n4c5ccccc5c5c6[se]c7ccc(-n8c9ccccc9c9cc(-n%10c%11ccccc%11c%11ccccc%11%10)ccc98)cc7c6ccc54)cc3)cc2)cc1 ; c1ccc(-c2ccc(-c3ccc(-n4c5ccccc5c5c6[se]c7cc(-n8c9ccccc9c9cc(-n%10c%11ccccc%11c%11ccccc%11%10)ccc98)ccc7c6ccc54)cc3)cc2)cc1 ; c1ccc(-c2ccc(-c3ccc(-n4c5ccccc5c5c6[se]c7c(-n8c9ccccc9c9cc(-n%10c%11ccccc%11c%11ccccc%11%10)ccc98)cccc7c6ccc54)cc3)cc2)cc1 ; c1ccc(-c2ccc(-c3cccc(-n4c5ccccc5c5c6[se]c7cccc(-n8c9ccccc9c9cc(-n%10c%11ccccc%11c%11ccccc%11%10)ccc98)c7c6ccc54)c3)cc2)cc1]

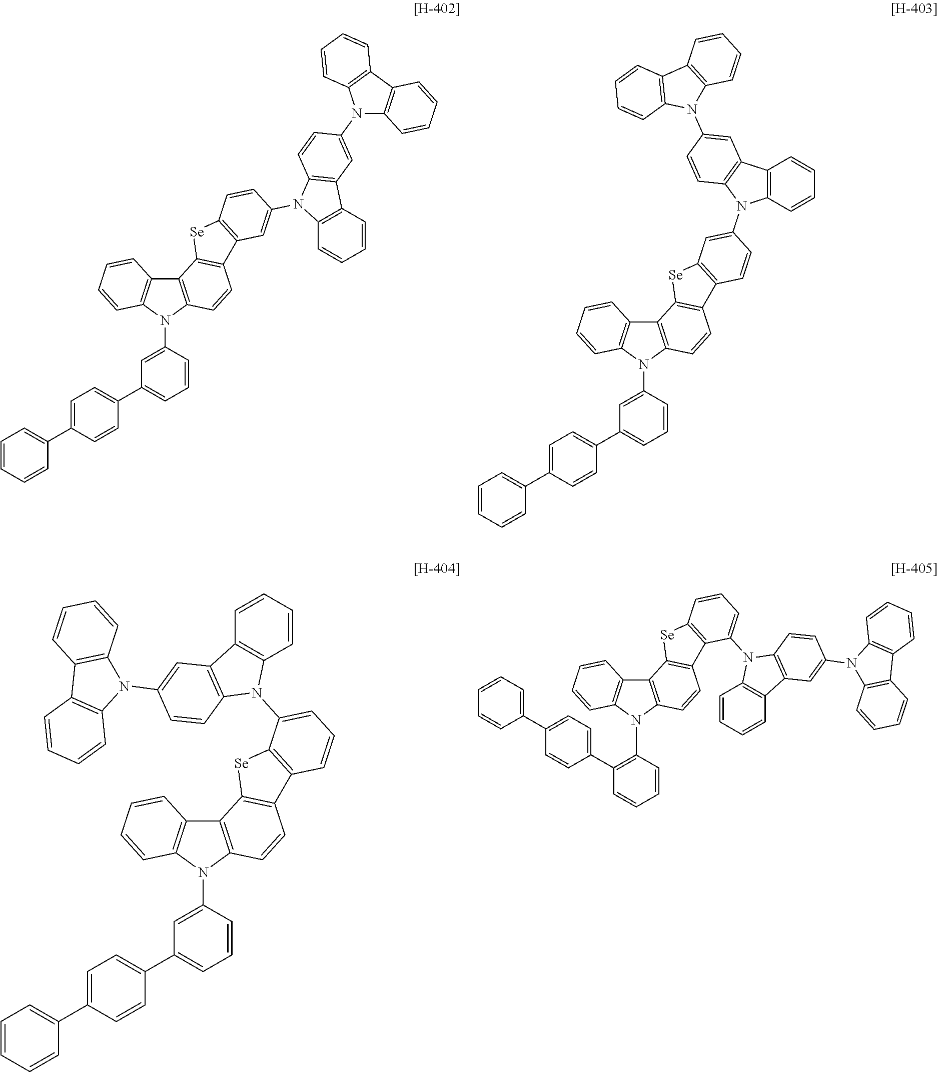 [SMILES: c1ccc(-c2ccc(-c3cccc(-n4c5ccccc5c5c6[se]c7ccc(-n8c9ccccc9c9cc(-n%10c%11ccccc%11c%11ccccc%11%10)ccc98)cc7c6ccc54)c3)cc2)cc1 ; c1ccc(-c2ccc(-c3cccc(-n4c5ccccc5c5c6[se]c7cc(-n8c9ccccc9c9cc(-n%10c%11ccccc%11c%11ccccc%11%10)ccc98)ccc7c6ccc54)c3)cc2)cc1 ; c1ccc(-c2ccc(-c3cccc(-n4c5ccccc5c5c6[se]c7c(-n8c9ccccc9c9cc(-n%10c%11ccccc%11c%11ccccc%11%10)ccc98)cccc7c6ccc54)c3)cc2)cc1 ; c1ccc(-c2ccc(-c3ccccc3-n3c4ccccc4c4c5[se]c6cccc(-n7c8ccccc8c8cc(-n9c%10ccccc%10c%10ccccc%109)ccc87)c6c5ccc43)cc2)cc1]

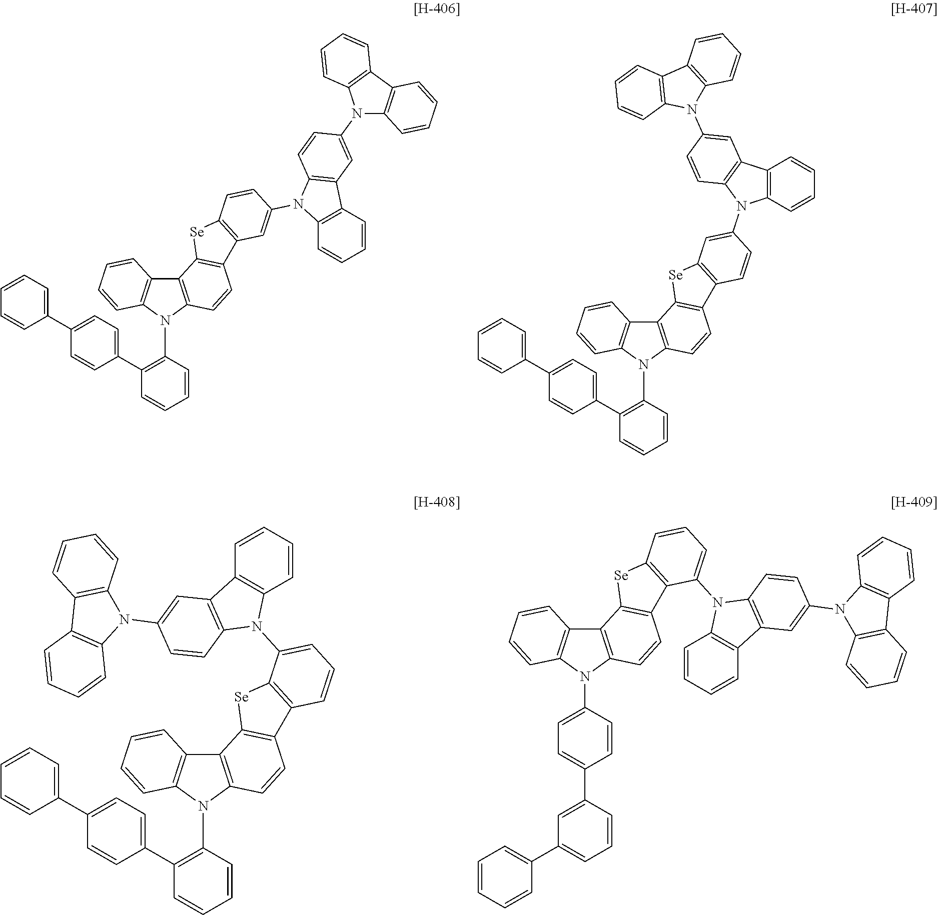 [SMILES: c1ccc(-c2ccc(-c3ccccc3-n3c4ccccc4c4c5[se]c6ccc(-n7c8ccccc8c8cc(-n9c%10ccccc%10c%10ccccc%109)ccc87)cc6c5ccc43)cc2)cc1 ; c1ccc(-c2ccc(-c3ccccc3-n3c4ccccc4c4c5[se]c6cc(-n7c8ccccc8c8cc(-n9c%10ccccc%10c%10ccccc%109)ccc87)ccc6c5ccc43)cc2)cc1 ; c1ccc(-c2ccc(-c3ccccc3-n3c4ccccc4c4c5[se]c6c(-n7c8ccccc8c8cc(-n9c%10ccccc%10c%10ccccc%109)ccc87)cccc6c5ccc43)cc2)cc1 ; c1ccc(-c2cccc(-c3ccc(-n4c5ccccc5c5c6[se]c7cccc(-n8c9ccccc9c9cc(-n%10c%11ccccc%11c%11ccccc%11%10)ccc98)c7c6ccc54)cc3)c2)cc1]

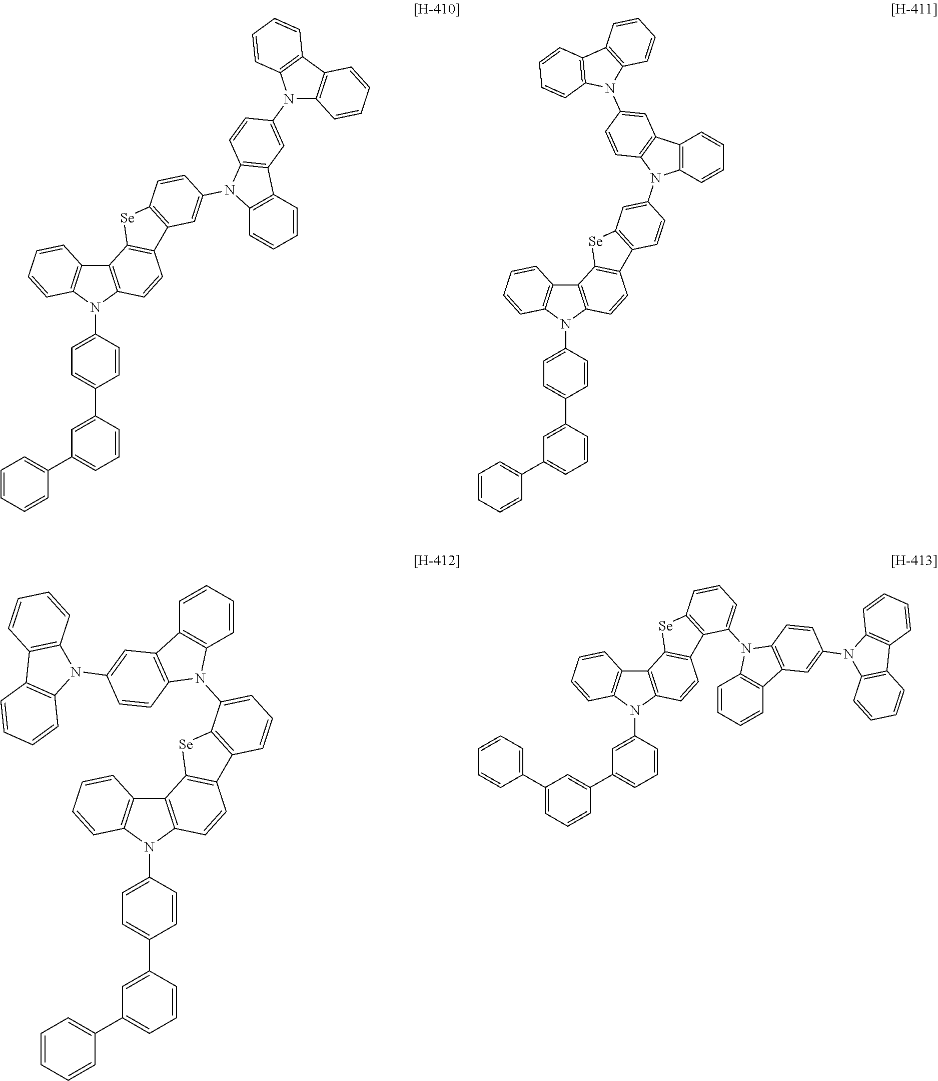 [SMILES: c1ccc(-c2cccc(-c3ccc(-n4c5ccccc5c5c6[se]c7ccc(-n8c9ccccc9c9cc(-n%10c%11ccccc%11c%11ccccc%11%10)ccc98)cc7c6ccc54)cc3)c2)cc1 ; c1ccc(-c2cccc(-c3ccc(-n4c5ccccc5c5c6[se]c7cc(-n8c9ccccc9c9cc(-n%10c%11ccccc%11c%11ccccc%11%10)ccc98)ccc7c6ccc54)cc3)c2)cc1 ; c1ccc(-c2cccc(-c3ccc(-n4c5ccccc5c5c6[se]c7c(-n8c9ccccc9c9cc(-n%10c%11ccccc%11c%11ccccc%11%10)ccc98)cccc7c6ccc54)cc3)c2)cc1 ; c1ccc(-c2cccc(-c3cccc(-n4c5ccccc5c5c6[se]c7cccc(-n8c9ccccc9c9cc(-n%10c%11ccccc%11c%11ccccc%11%10)ccc98)c7c6ccc54)c3)c2)cc1]

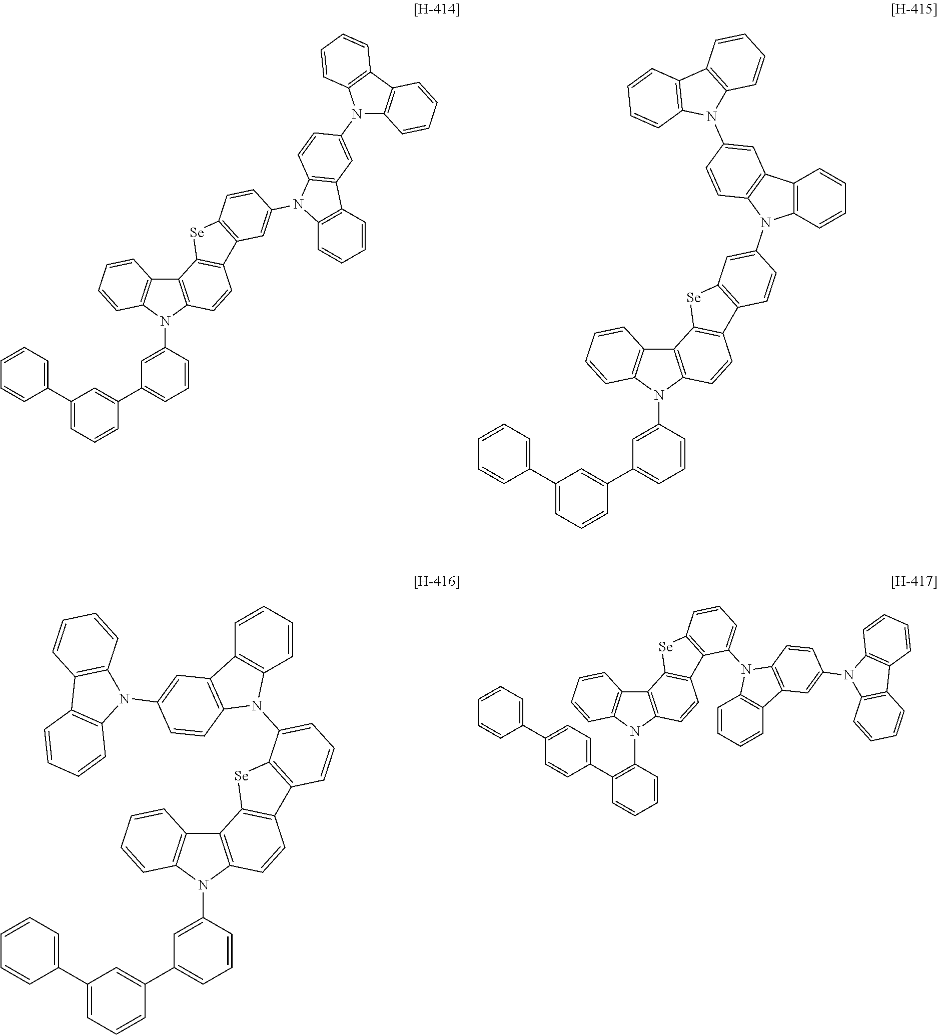 [SMILES: c1ccc(-c2cccc(-c3cccc(-n4c5ccccc5c5c6[se]c7ccc(-n8c9ccccc9c9cc(-n%10c%11ccccc%11c%11ccccc%11%10)ccc98)cc7c6ccc54)c3)c2)cc1 ; c1ccc(-c2cccc(-c3cccc(-n4c5ccccc5c5c6[se]c7cc(-n8c9ccccc9c9cc(-n%10c%11ccccc%11c%11ccccc%11%10)ccc98)ccc7c6ccc54)c3)c2)cc1 ; c1ccc(-c2cccc(-c3cccc(-n4c5ccccc5c5c6[se]c7c(-n8c9ccccc9c9cc(-n%10c%11ccccc%11c%11ccccc%11%10)ccc98)cccc7c6ccc54)c3)c2)cc1 ; c1ccc(-c2ccc(-c3ccccc3-n3c4ccccc4c4c5[se]c6cccc(-n7c8ccccc8c8cc(-n9c%10ccccc%10c%10ccccc%109)ccc87)c6c5ccc43)cc2)cc1]

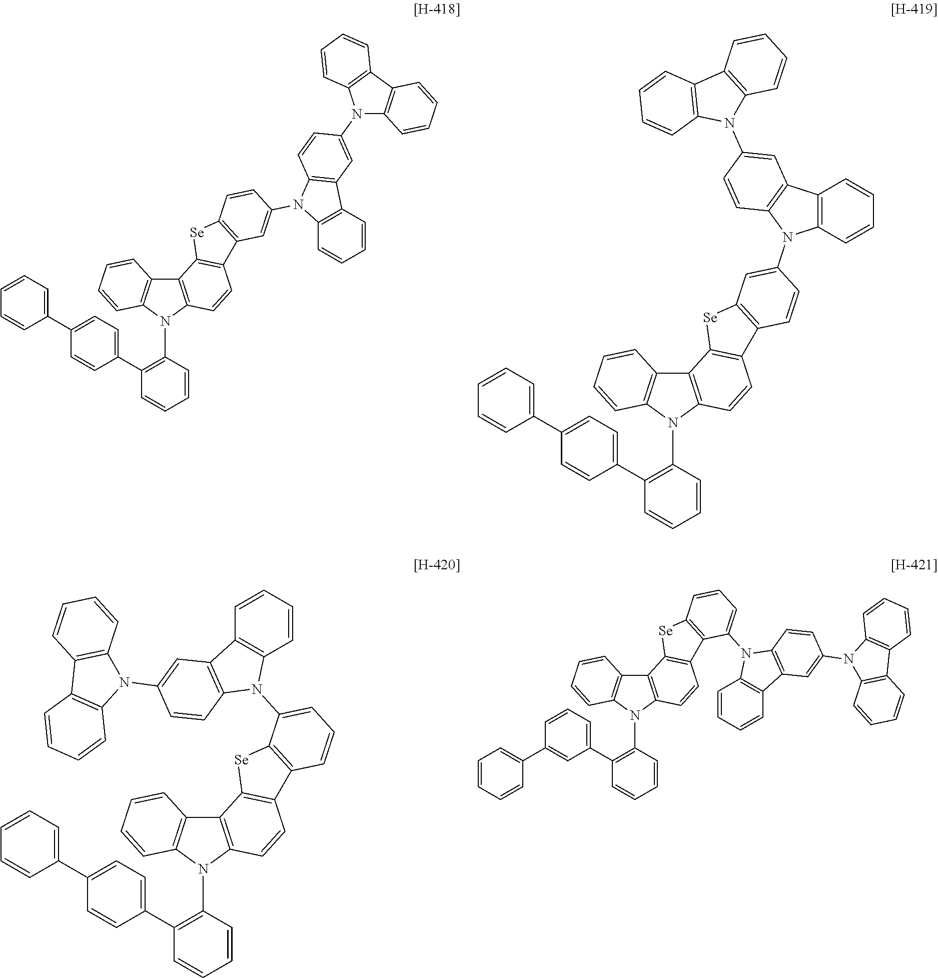 [SMILES: c1ccc(-c2ccc(-c3ccccc3-n3c4ccccc4c4c5[se]c6ccc(-n7c8ccccc8c8cc(-n9c%10ccccc%10c%10ccccc%109)ccc87)cc6c5ccc43)cc2)cc1 ; c1ccc(-c2ccc(-c3ccccc3-n3c4ccccc4c4c5[se]c6cc(-n7c8ccccc8c8cc(-n9c%10ccccc%10c%10ccccc%109)ccc87)ccc6c5ccc43)cc2)cc1 ; c1ccc(-c2ccc(-c3ccccc3-n3c4ccccc4c4c5[se]c6c(-n7c8ccccc8c8cc(-n9c%10ccccc%10c%10ccccc%109)ccc87)cccc6c5ccc43)cc2)cc1 ; c1ccc(-c2cccc(-c3ccccc3-n3c4ccccc4c4c5[se]c6cccc(-n7c8ccccc8c8cc(-n9c%10ccccc%10c%10ccccc%109)ccc87)c6c5ccc43)c2)cc1]

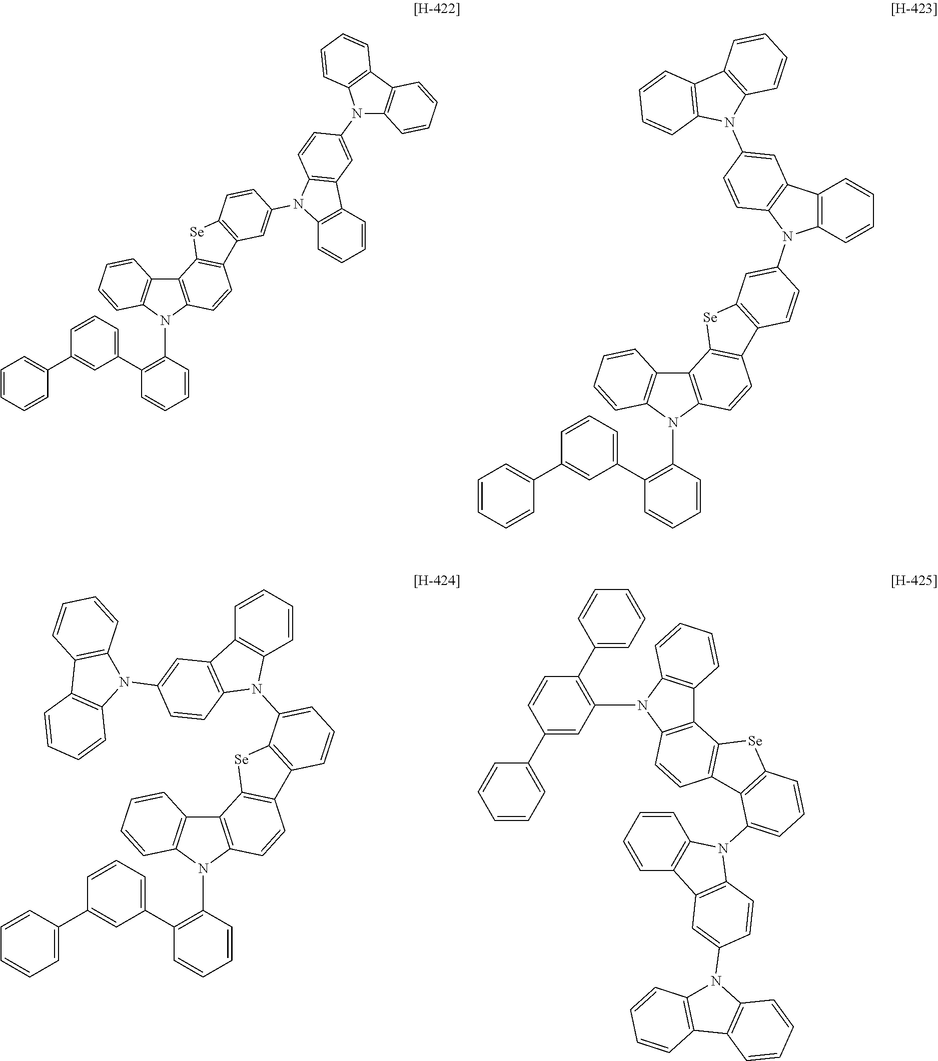 [SMILES: c1ccc(-c2cccc(-c3ccccc3-n3c4ccccc4c4c5[se]c6ccc(-n7c8ccccc8c8cc(-n9c%10ccccc%10c%10ccccc%109)ccc87)cc6c5ccc43)c2)cc1 ; c1ccc(-c2cccc(-c3ccccc3-n3c4ccccc4c4c5[se]c6cc(-n7c8ccccc8c8cc(-n9c%10ccccc%10c%10ccccc%109)ccc87)ccc6c5ccc43)c2)cc1 ; c1ccc(-c2cccc(-c3ccccc3-n3c4ccccc4c4c5[se]c6c(-n7c8ccccc8c8cc(-n9c%10ccccc%10c%10ccccc%109)ccc87)cccc6c5ccc43)c2)cc1 ; c1ccc(-c2ccc(-c3ccccc3)c(-n3c4ccccc4c4c5[se]c6cccc(-n7c8ccccc8c8cc(-n9c%10ccccc%10c%10ccccc%109)ccc87)c6c5ccc43)c2)cc1]

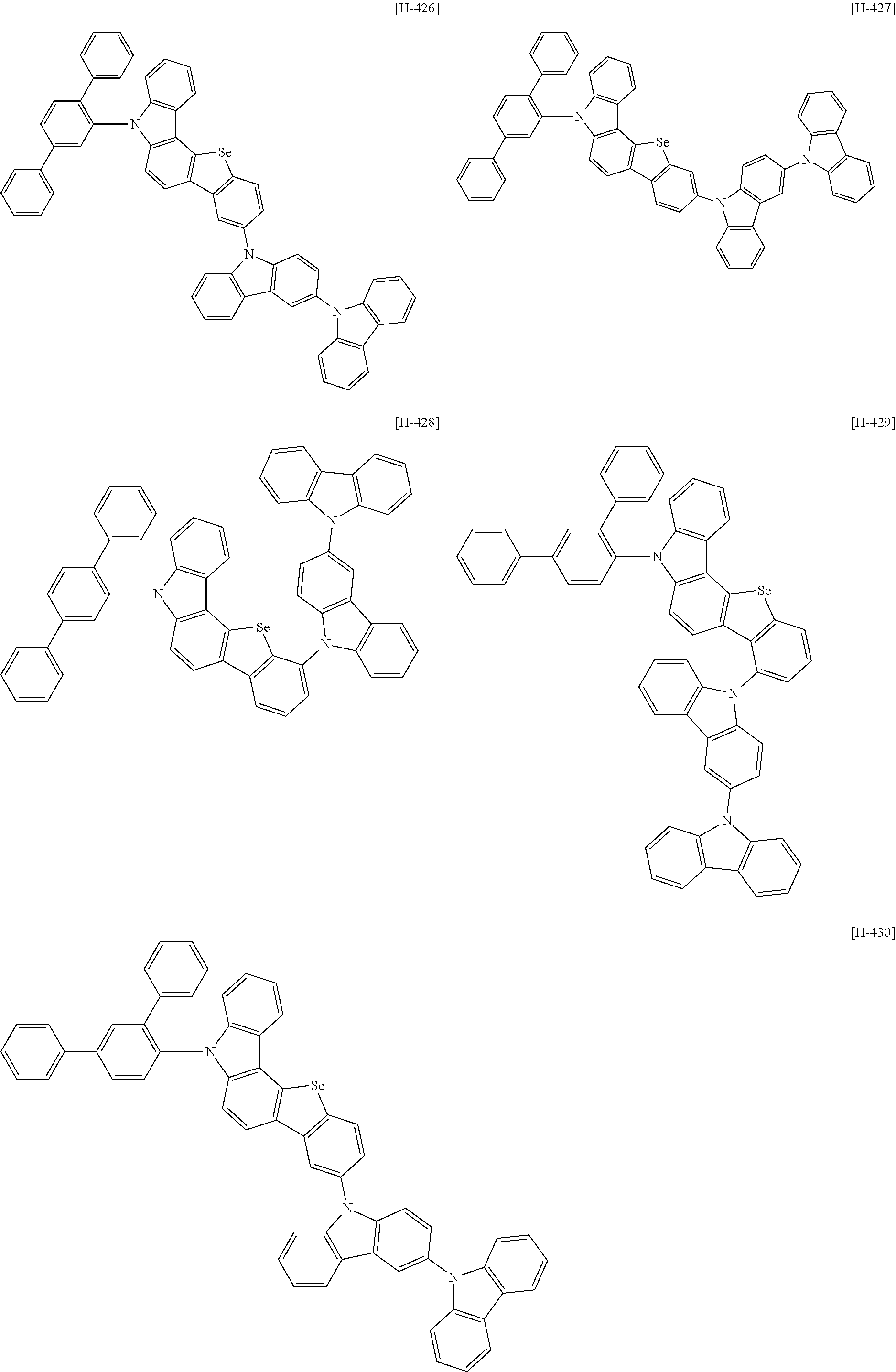

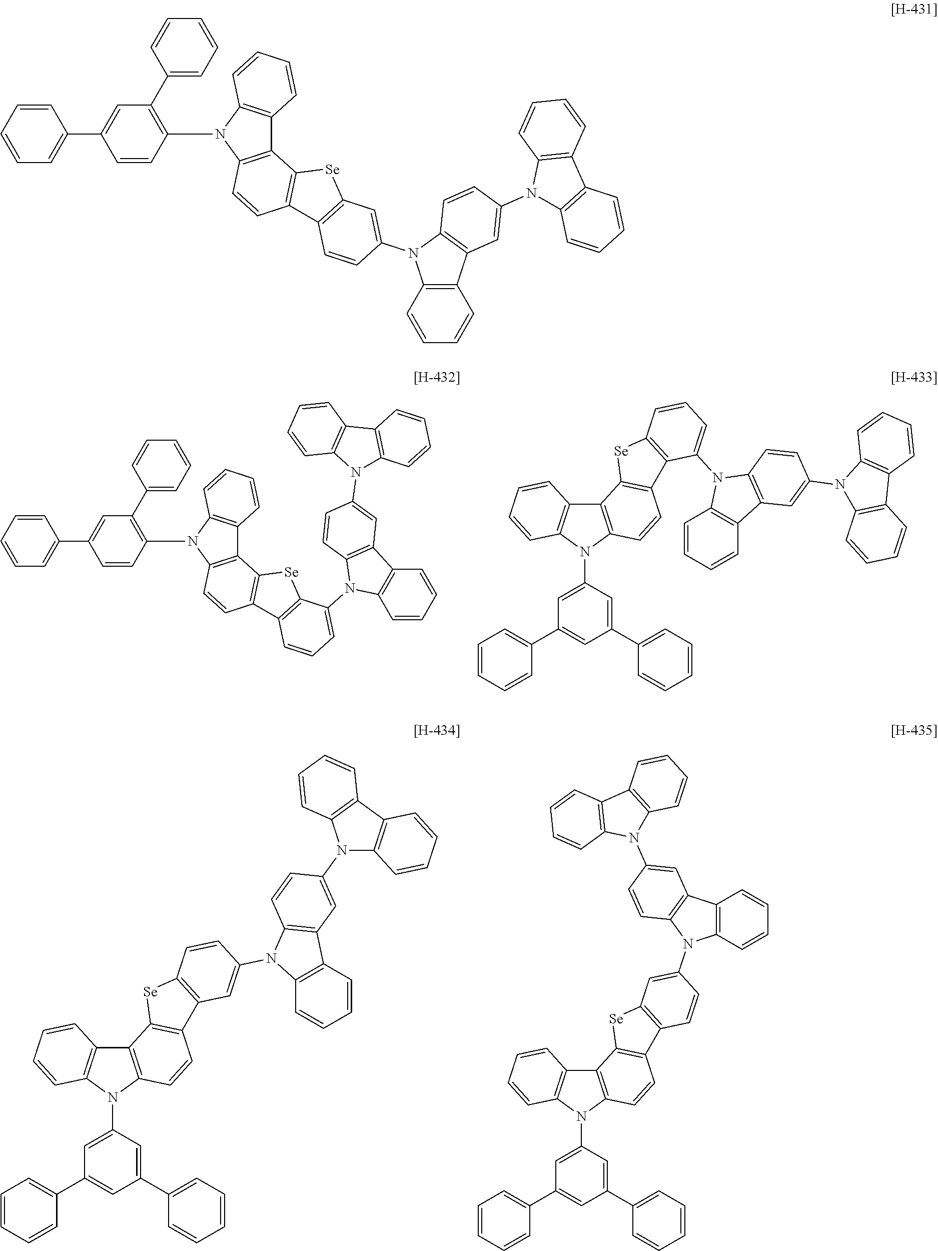

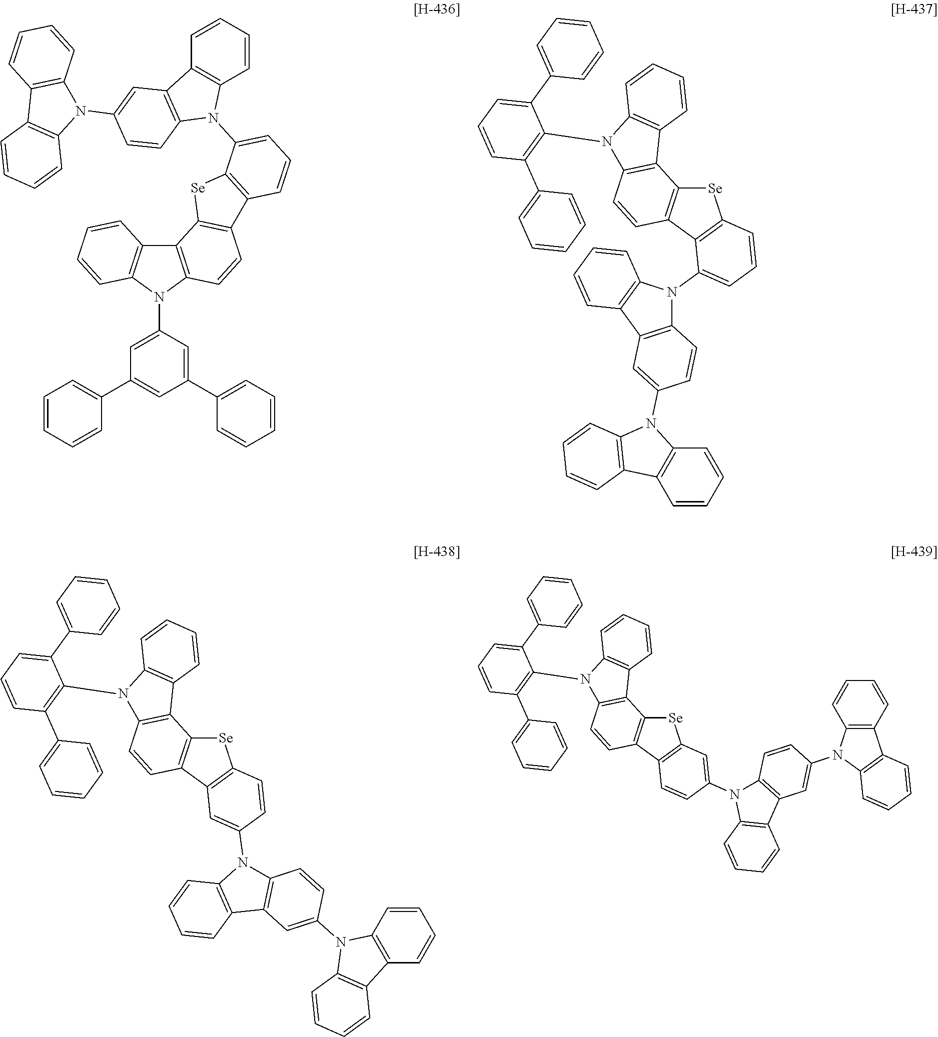 [SMILES: c1ccc(-c2cc(-c3ccccc3)cc(-n3c4ccccc4c4c5[se]c6c(-n7c8ccccc8c8cc(-n9c%10ccccc%10c%10ccccc%109)ccc87)cccc6c5ccc43)c2)cc1 ; c1ccc(-c2cccc(-c3ccccc3)c2-n2c3ccccc3c3c4[se]c5cccc(-n6c7ccccc7c7cc(-n8c9ccccc9c9ccccc98)ccc76)c5c4ccc32)cc1 ; c1ccc(-c2cccc(-c3ccccc3)c2-n2c3ccccc3c3c4[se]c5ccc(-n6c7ccccc7c7cc(-n8c9ccccc9c9ccccc98)ccc76)cc5c4ccc32)cc1 ; c1ccc(-c2cccc(-c3ccccc3)c2-n2c3ccccc3c3c4[se]c5cc(-n6c7ccccc7c7cc(-n8c9ccccc9c9ccccc98)ccc76)ccc5c4ccc32)cc1]

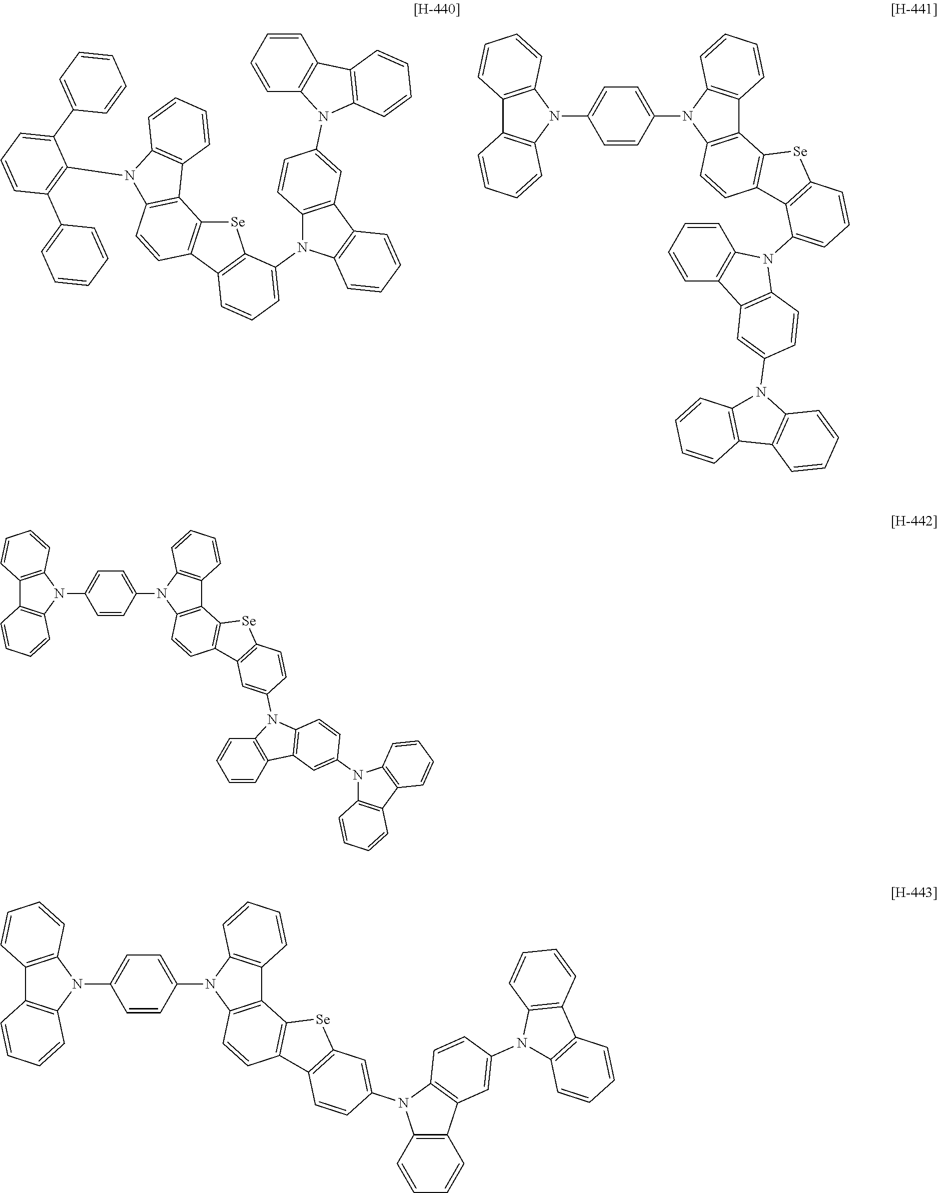 [SMILES: c1ccc(-c2cccc(-c3ccccc3)c2-n2c3ccccc3c3c4[se]c5c(-n6c7ccccc7c7cc(-n8c9ccccc9c9ccccc98)ccc76)cccc5c4ccc32)cc1 ; c1cc(-n2c3ccccc3c3cc(-n4c5ccccc5c5ccccc54)ccc32)c2c(c1)[se]c1c2ccc2c1c1ccccc1n2-c1ccc(-n2c3ccccc3c3ccccc32)cc1 ; c1ccc2c(c1)c1ccccc1n2-c1ccc(-n2c3ccccc3c3c4[se]c5ccc(-n6c7ccccc7c7cc(-n8c9ccccc9c9ccccc98)ccc76)cc5c4ccc32)cc1 ; c1ccc2c(c1)c1ccccc1n2-c1ccc(-n2c3ccccc3c3c4[se]c5cc(-n6c7ccccc7c7cc(-n8c9ccccc9c9ccccc98)ccc76)ccc5c4ccc32)cc1]

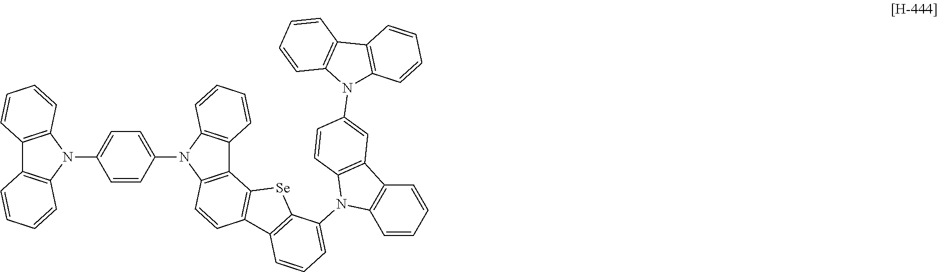 [SMILES: c1cc(-n2c3ccccc3c3cc(-n4c5ccccc5c5ccccc54)ccc32)c2[se]c3c(ccc4c3c3ccccc3n4-c3ccc(-n4c5ccccc5c5ccccc54)cc3)c2c1]

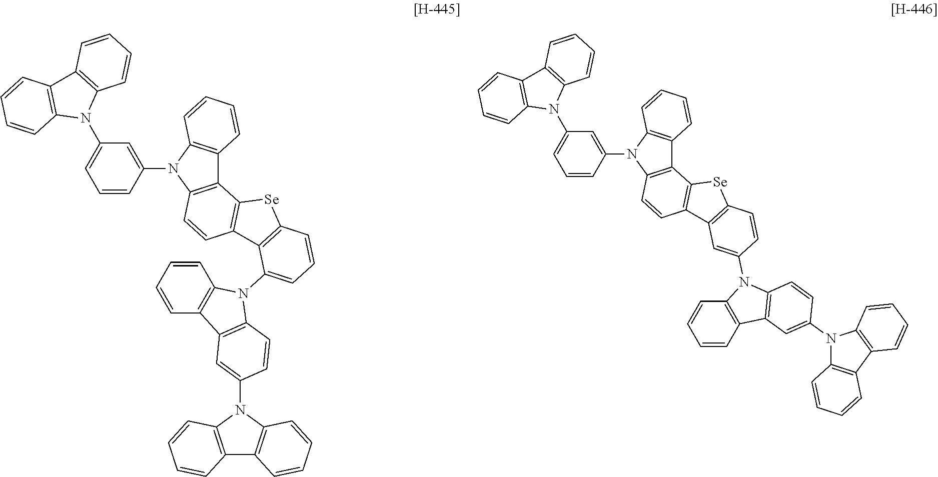 [SMILES: c1cc(-n2c3ccccc3c3ccccc32)cc(-n2c3ccccc3c3c4[se]c5cccc(-n6c7ccccc7c7cc(-n8c9ccccc9c9ccccc98)ccc76)c5c4ccc32)c1 ; c1cc(-n2c3ccccc3c3ccccc32)cc(-n2c3ccccc3c3c4[se]c5ccc(-n6c7ccccc7c7cc(-n8c9ccccc9c9ccccc98)ccc76)cc5c4ccc32)c1]

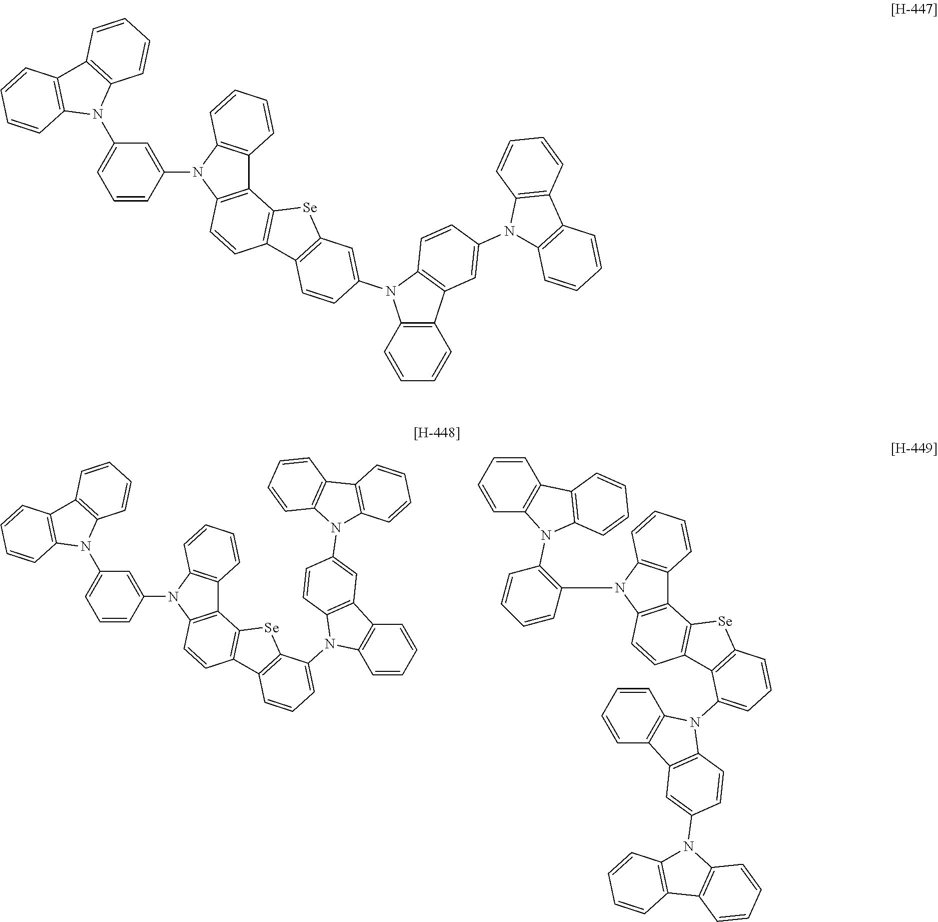 [SMILES: c1cc(-n2c3ccccc3c3ccccc32)cc(-n2c3ccccc3c3c4[se]c5cc(-n6c7ccccc7c7cc(-n8c9ccccc9c9ccccc98)ccc76)ccc5c4ccc32)c1 ; c1cc(-n2c3ccccc3c3ccccc32)cc(-n2c3ccccc3c3c4[se]c5c(-n6c7ccccc7c7cc(-n8c9ccccc9c9ccccc98)ccc76)cccc5c4ccc32)c1 ; c1ccc(-n2c3ccccc3c3c4[se]c5cccc(-n6c7ccccc7c7cc(-n8c9ccccc9c9ccccc98)ccc76)c5c4ccc32)c(-n2c3ccccc3c3ccccc32)c1]

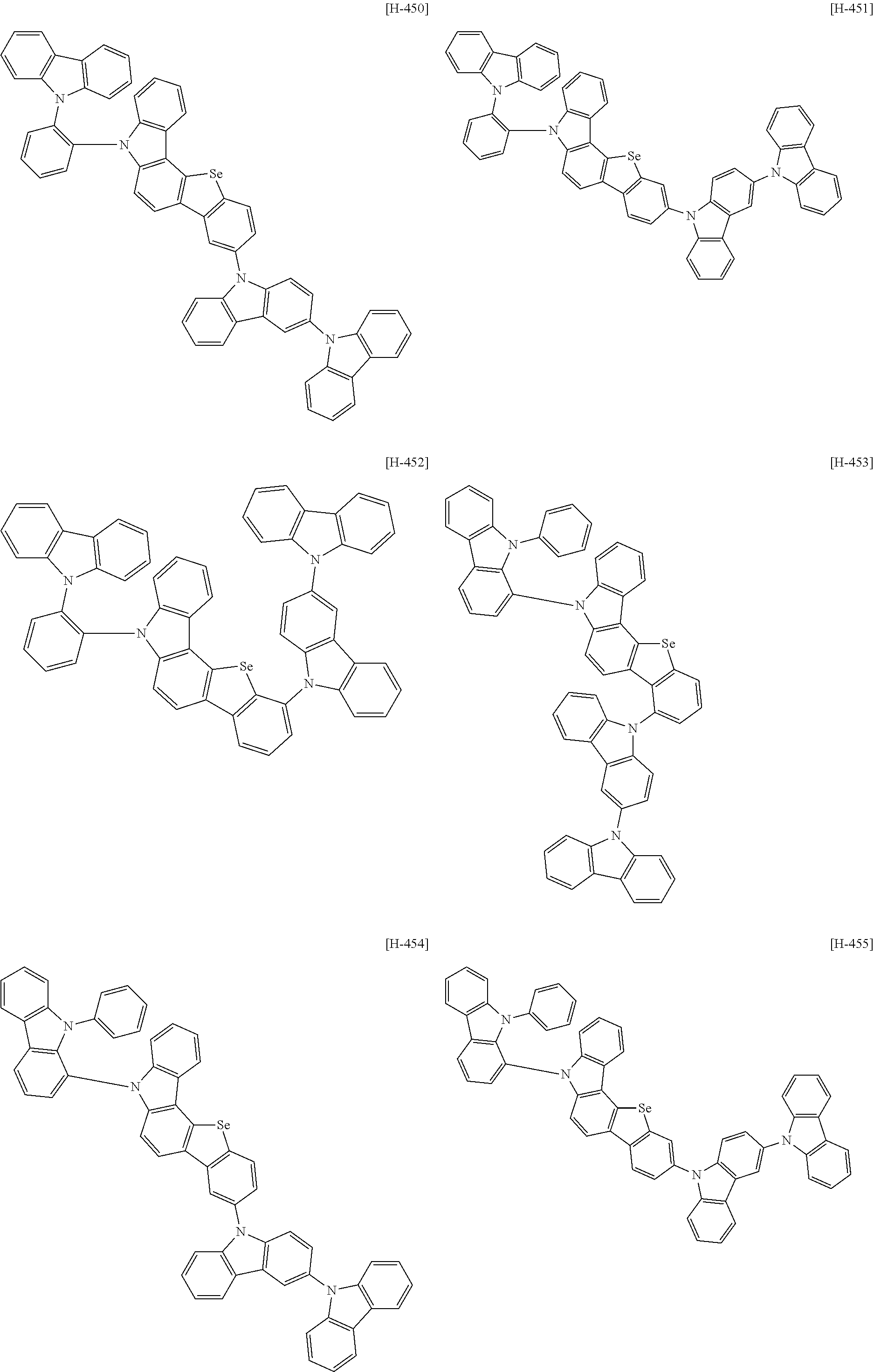

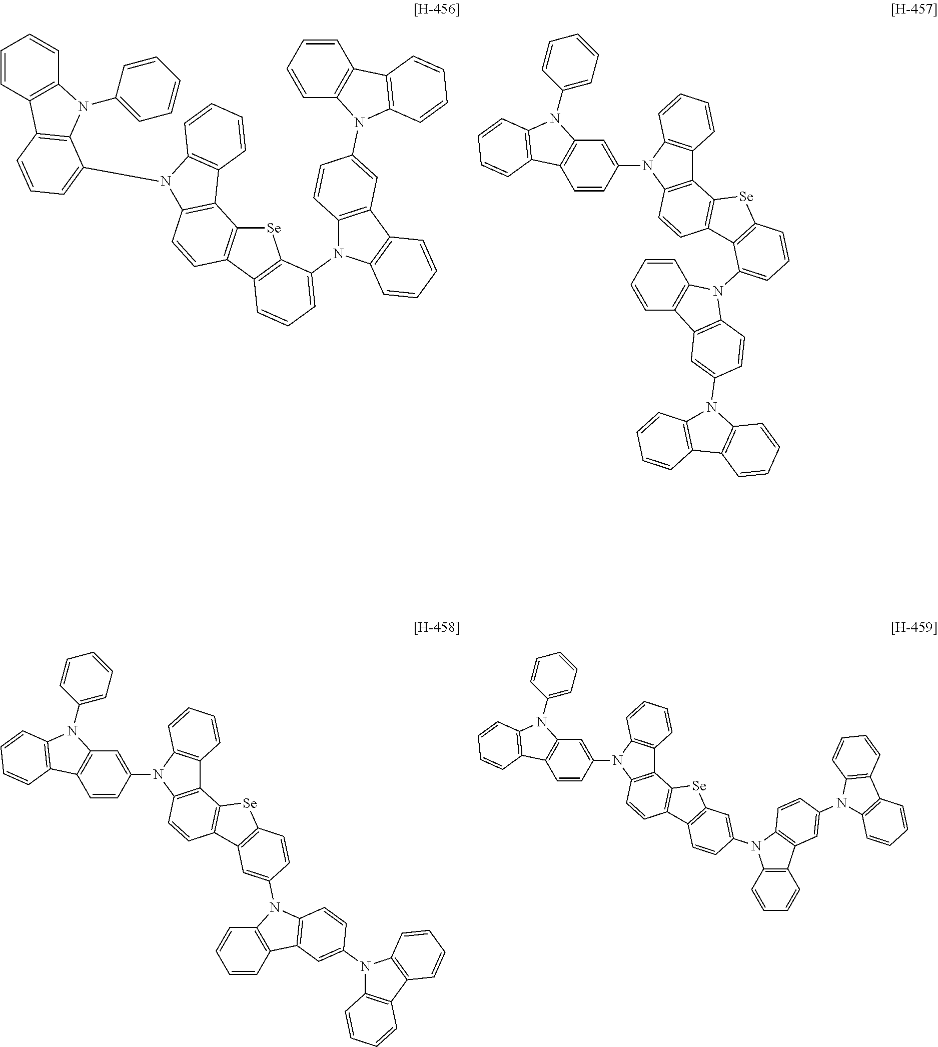 [SMILES: c1ccc(-n2c3ccccc3c3cccc(-n4c5ccccc5c5c6[se]c7c(-n8c9ccccc9c9cc(-n%10c%11ccccc%11c%11ccccc%11%10)ccc98)cccc7c6ccc54)c32)cc1 ; c1ccc(-n2c3ccccc3c3ccc(-n4c5ccccc5c5c6[se]c7cccc(-n8c9ccccc9c9cc(-n%10c%11ccccc%11c%11ccccc%11%10)ccc98)c7c6ccc54)cc32)cc1 ; c1ccc(-n2c3ccccc3c3ccc(-n4c5ccccc5c5c6[se]c7ccc(-n8c9ccccc9c9cc(-n%10c%11ccccc%11c%11ccccc%11%10)ccc98)cc7c6ccc54)cc32)cc1 ; c1ccc(-n2c3ccccc3c3ccc(-n4c5ccccc5c5c6[se]c7cc(-n8c9ccccc9c9cc(-n%10c%11ccccc%11c%11ccccc%11%10)ccc98)ccc7c6ccc54)cc32)cc1]

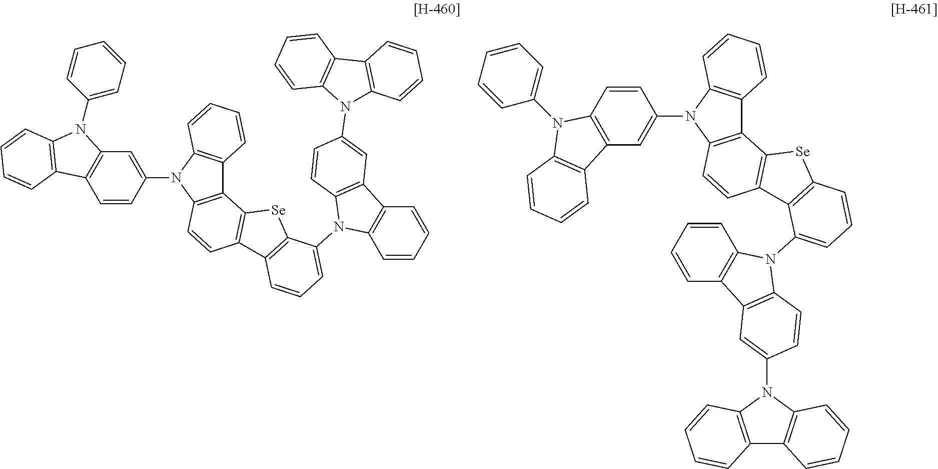 [SMILES: c1ccc(-n2c3ccccc3c3ccc(-n4c5ccccc5c5c6[se]c7c(-n8c9ccccc9c9cc(-n%10c%11ccccc%11c%11ccccc%11%10)ccc98)cccc7c6ccc54)cc32)cc1 ; c1ccc(-n2c3ccccc3c3cc(-n4c5ccccc5c5c6[se]c7cccc(-n8c9ccccc9c9cc(-n%10c%11ccccc%11c%11ccccc%11%10)ccc98)c7c6ccc54)ccc32)cc1]

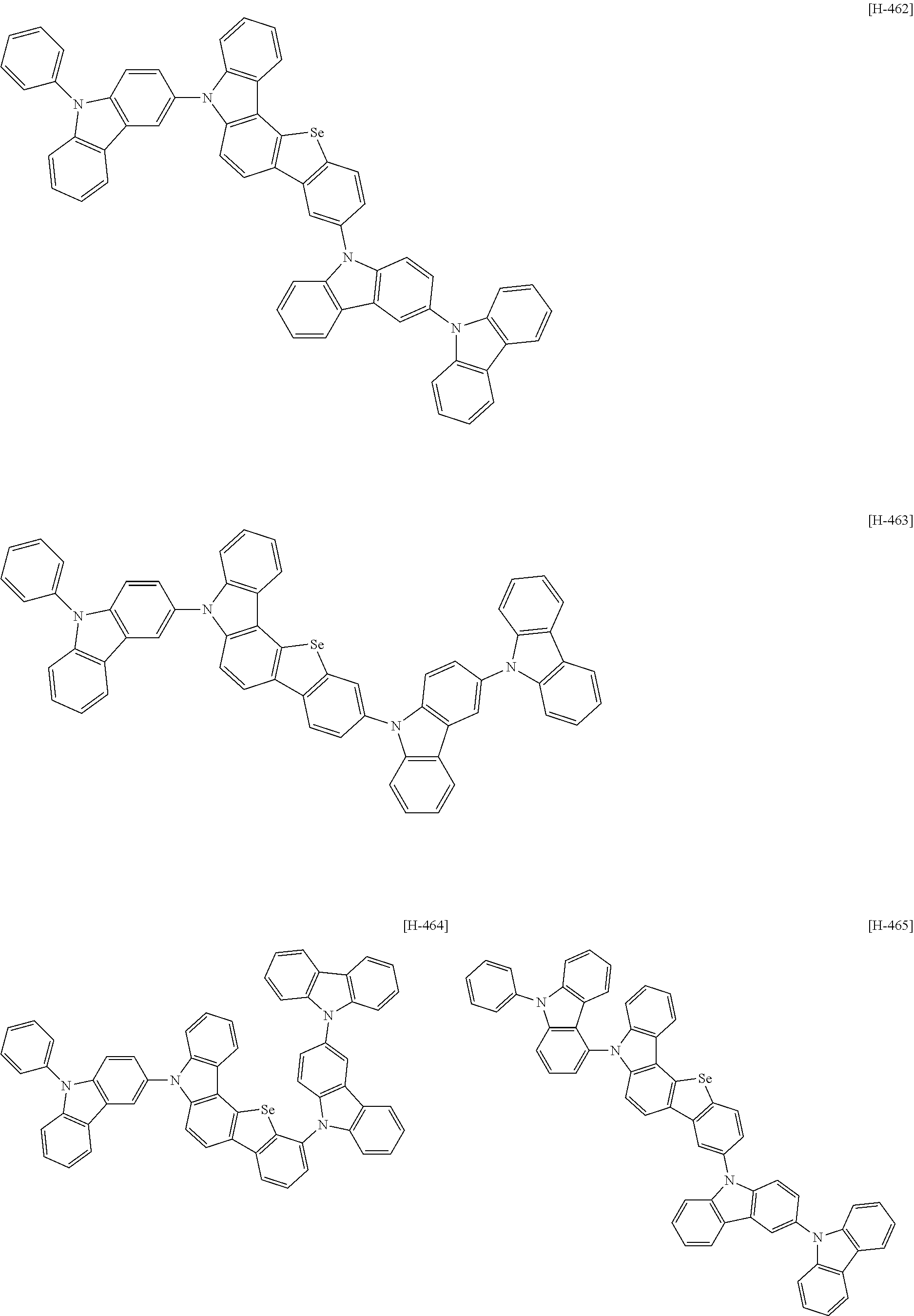 [SMILES: c1ccc(-n2c3ccccc3c3cc(-n4c5ccccc5c5c6[se]c7ccc(-n8c9ccccc9c9cc(-n%10c%11ccccc%11c%11ccccc%11%10)ccc98)cc7c6ccc54)ccc32)cc1 ; c1ccc(-n2c3ccccc3c3cc(-n4c5ccccc5c5c6[se]c7cc(-n8c9ccccc9c9cc(-n%10c%11ccccc%11c%11ccccc%11%10)ccc98)ccc7c6ccc54)ccc32)cc1 ; c1ccc(-n2c3ccccc3c3cc(-n4c5ccccc5c5c6[se]c7c(-n8c9ccccc9c9cc(-n%10c%11ccccc%11c%11ccccc%11%10)ccc98)cccc7c6ccc54)ccc32)cc1 ; c1ccc(-n2c3ccccc3c3c(-n4c5ccccc5c5c6[se]c7ccc(-n8c9ccccc9c9cc(-n%10c%11ccccc%11c%11ccccc%11%10)ccc98)cc7c6ccc54)cccc32)cc1]

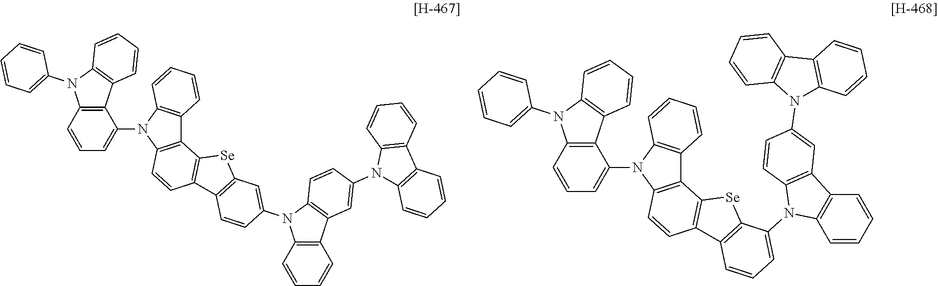 [SMILES: c1ccc(-n2c3ccccc3c3c(-n4c5ccccc5c5c6[se]c7cc(-n8c9ccccc9c9cc(-n%10c%11ccccc%11c%11ccccc%11%10)ccc98)ccc7c6ccc54)cccc32)cc1 ; c1ccc(-n2c3ccccc3c3c(-n4c5ccccc5c5c6[se]c7c(-n8c9ccccc9c9cc(-n%10c%11ccccc%11c%11ccccc%11%10)ccc98)cccc7c6ccc54)cccc32)cc1]

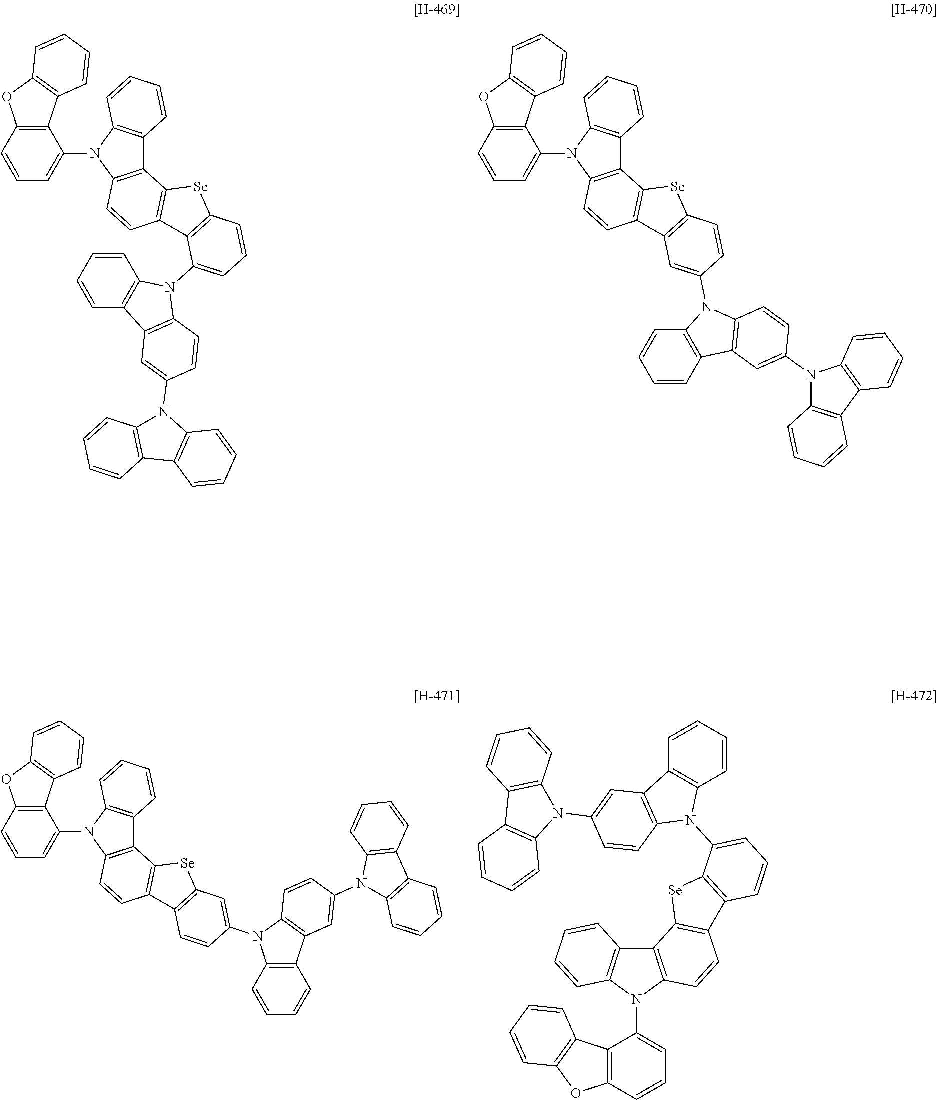 [SMILES: c1ccc2c(c1)oc1cccc(-n3c4ccccc4c4c5[se]c6cccc(-n7c8ccccc8c8cc(-n9c%10ccccc%10c%10ccccc%109)ccc87)c6c5ccc43)c12 ; c1ccc2c(c1)oc1cccc(-n3c4ccccc4c4c5[se]c6ccc(-n7c8ccccc8c8cc(-n9c%10ccccc%10c%10ccccc%109)ccc87)cc6c5ccc43)c12 ; c1ccc2c(c1)oc1cccc(-n3c4ccccc4c4c5[se]c6cc(-n7c8ccccc8c8cc(-n9c%10ccccc%10c%10ccccc%109)ccc87)ccc6c5ccc43)c12 ; c1ccc2c(c1)oc1cccc(-n3c4ccccc4c4c5[se]c6c(-n7c8ccccc8c8cc(-n9c%10ccccc%10c%10ccccc%109)ccc87)cccc6c5ccc43)c12]

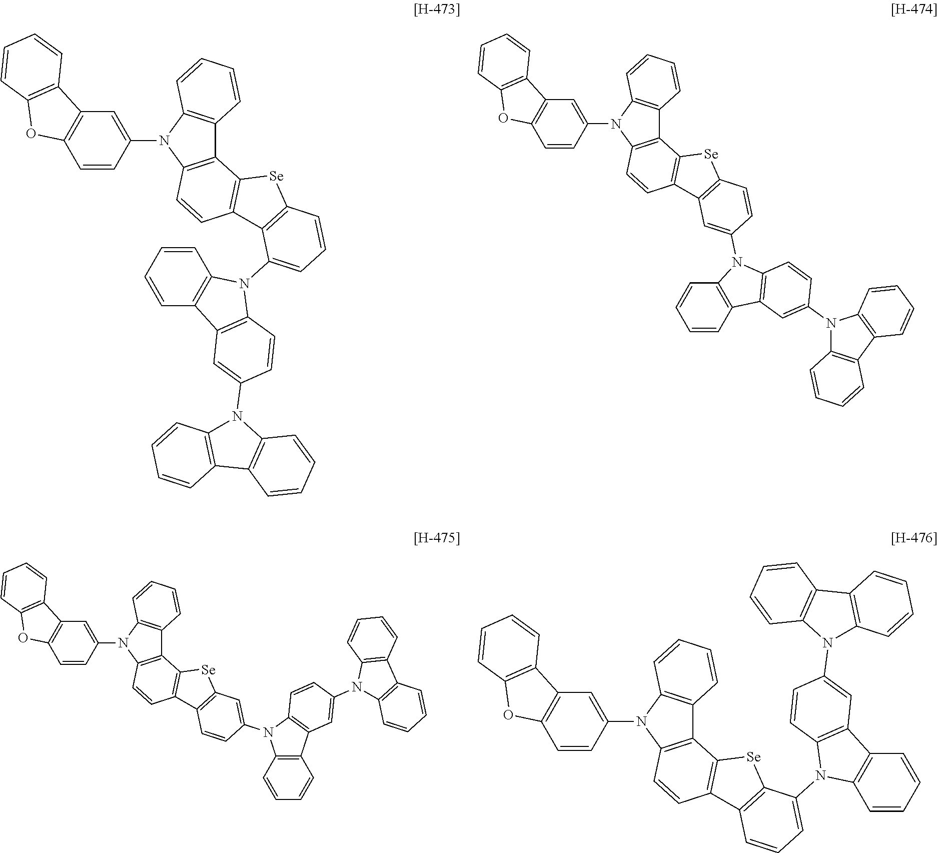 [SMILES: c1ccc2c(c1)oc1ccc(-n3c4ccccc4c4c5[se]c6cccc(-n7c8ccccc8c8cc(-n9c%10ccccc%10c%10ccccc%109)ccc87)c6c5ccc43)cc12 ; c1ccc2c(c1)oc1ccc(-n3c4ccccc4c4c5[se]c6ccc(-n7c8ccccc8c8cc(-n9c%10ccccc%10c%10ccccc%109)ccc87)cc6c5ccc43)cc12 ; c1ccc2c(c1)oc1ccc(-n3c4ccccc4c4c5[se]c6cc(-n7c8ccccc8c8cc(-n9c%10ccccc%10c%10ccccc%109)ccc87)ccc6c5ccc43)cc12 ; c1ccc2c(c1)oc1ccc(-n3c4ccccc4c4c5[se]c6c(-n7c8ccccc8c8cc(-n9c%10ccccc%10c%10ccccc%109)ccc87)cccc6c5ccc43)cc12]

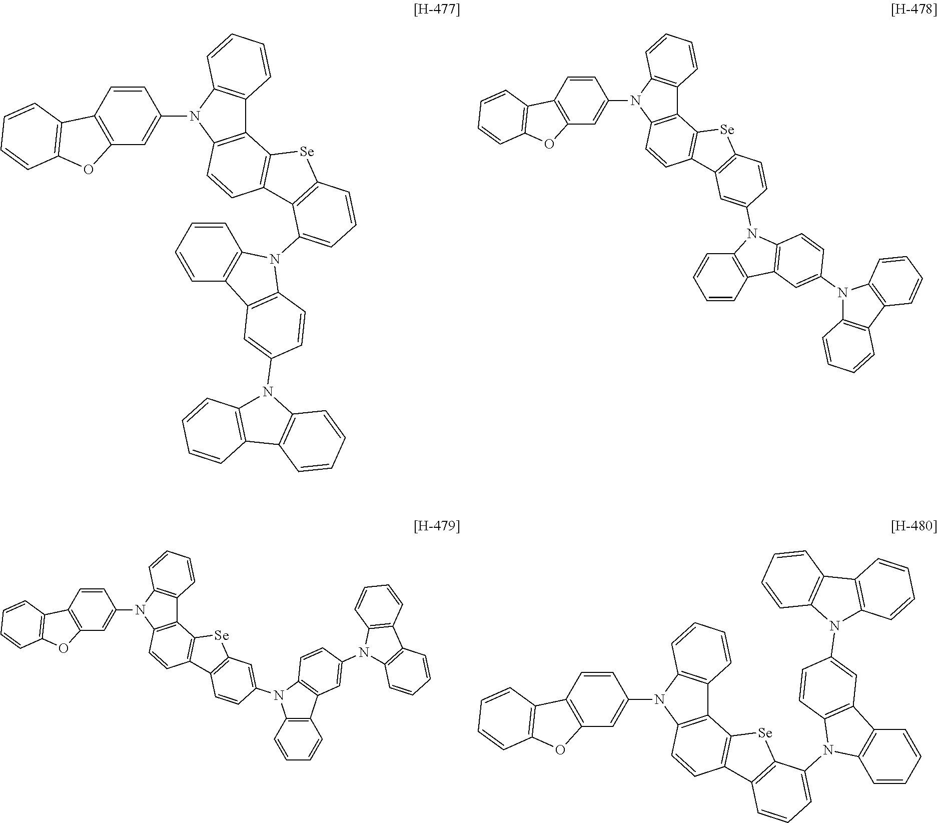 [SMILES: c1ccc2c(c1)oc1cc(-n3c4ccccc4c4c5[se]c6cccc(-n7c8ccccc8c8cc(-n9c%10ccccc%10c%10ccccc%109)ccc87)c6c5ccc43)ccc12 ; c1ccc2c(c1)oc1cc(-n3c4ccccc4c4c5[se]c6ccc(-n7c8ccccc8c8cc(-n9c%10ccccc%10c%10ccccc%109)ccc87)cc6c5ccc43)ccc12 ; c1ccc2c(c1)oc1cc(-n3c4ccccc4c4c5[se]c6cc(-n7c8ccccc8c8cc(-n9c%10ccccc%10c%10ccccc%109)ccc87)ccc6c5ccc43)ccc12 ; c1ccc2c(c1)oc1cc(-n3c4ccccc4c4c5[se]c6c(-n7c8ccccc8c8cc(-n9c%10ccccc%10c%10ccccc%109)ccc87)cccc6c5ccc43)ccc12]

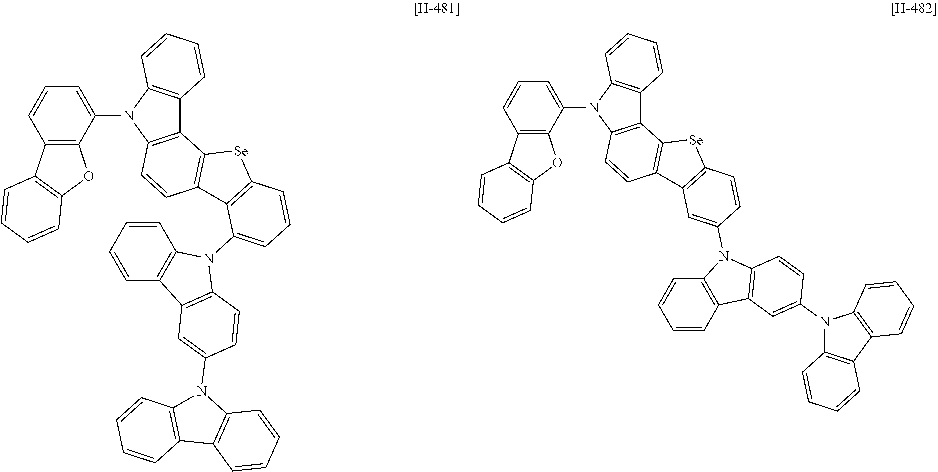 [SMILES: c1ccc2c(c1)oc1c(-n3c4ccccc4c4c5[se]c6cccc(-n7c8ccccc8c8cc(-n9c%10ccccc%10c%10ccccc%109)ccc87)c6c5ccc43)cccc12 ; c1ccc2c(c1)oc1c(-n3c4ccccc4c4c5[se]c6ccc(-n7c8ccccc8c8cc(-n9c%10ccccc%10c%10ccccc%109)ccc87)cc6c5ccc43)cccc12]

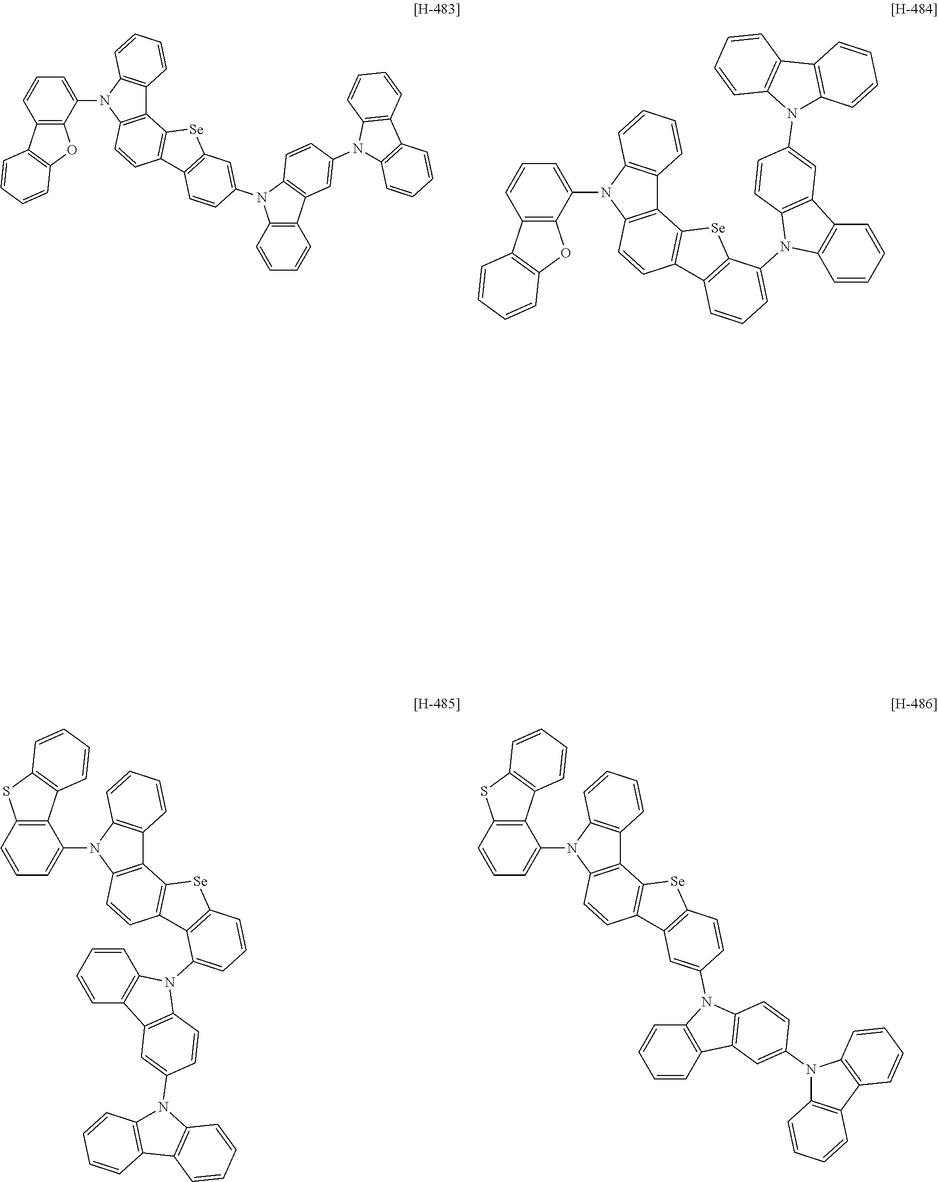 [SMILES: c1ccc2c(c1)oc1c(-n3c4ccccc4c4c5[se]c6cc(-n7c8ccccc8c8cc(-n9c%10ccccc%10c%10ccccc%109)ccc87)ccc6c5ccc43)cccc12 ; c1ccc2c(c1)oc1c(-n3c4ccccc4c4c5[se]c6c(-n7c8ccccc8c8cc(-n9c%10ccccc%10c%10ccccc%109)ccc87)cccc6c5ccc43)cccc12 ; c1ccc2c(c1)sc1cccc(-n3c4ccccc4c4c5[se]c6cccc(-n7c8ccccc8c8cc(-n9c%10ccccc%10c%10ccccc%109)ccc87)c6c5ccc43)c12 ; c1ccc2c(c1)sc1cccc(-n3c4ccccc4c4c5[se]c6ccc(-n7c8ccccc8c8cc(-n9c%10ccccc%10c%10ccccc%109)ccc87)cc6c5ccc43)c12]

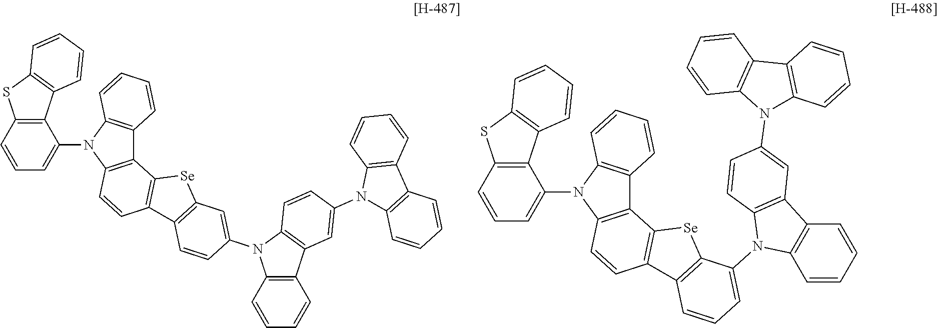

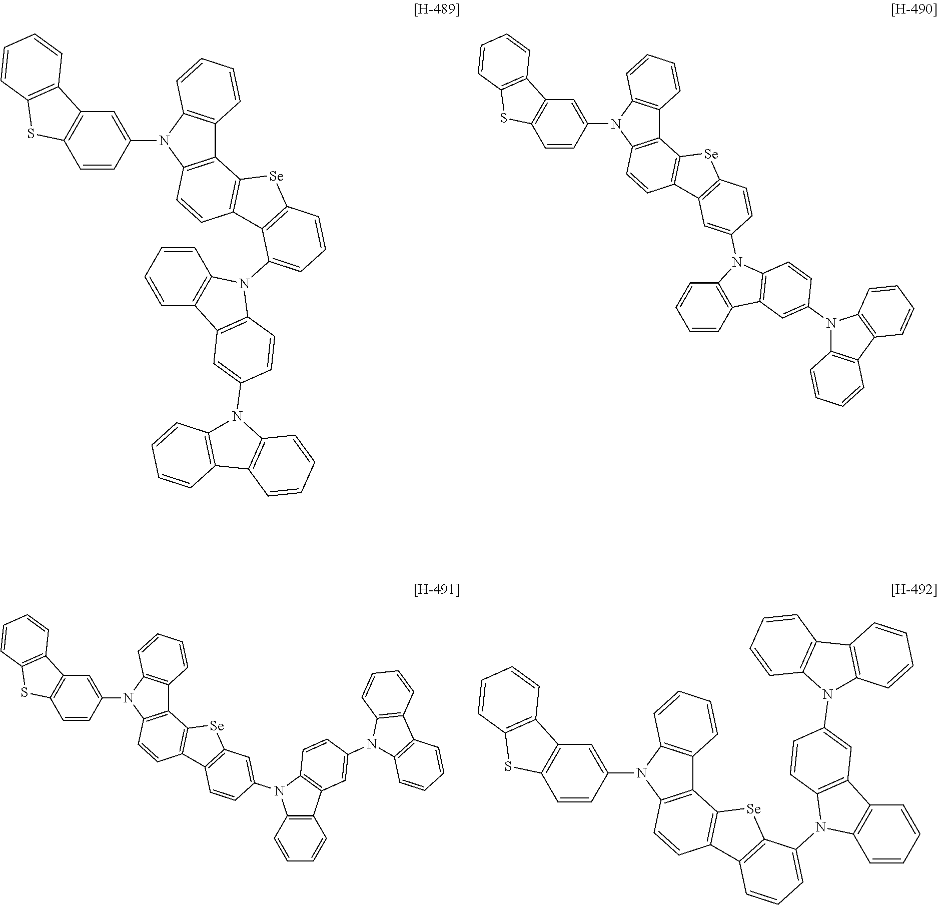 [SMILES: c1ccc2c(c1)sc1ccc(-n3c4ccccc4c4c5[se]c6cccc(-n7c8ccccc8c8cc(-n9c%10ccccc%10c%10ccccc%109)ccc87)c6c5ccc43)cc12 ; c1ccc2c(c1)sc1ccc(-n3c4ccccc4c4c5[se]c6ccc(-n7c8ccccc8c8cc(-n9c%10ccccc%10c%10ccccc%109)ccc87)cc6c5ccc43)cc12 ; c1ccc2c(c1)sc1ccc(-n3c4ccccc4c4c5[se]c6cc(-n7c8ccccc8c8cc(-n9c%10ccccc%10c%10ccccc%109)ccc87)ccc6c5ccc43)cc12 ; c1ccc2c(c1)sc1ccc(-n3c4ccccc4c4c5[se]c6c(-n7c8ccccc8c8cc(-n9c%10ccccc%10c%10ccccc%109)ccc87)cccc6c5ccc43)cc12]

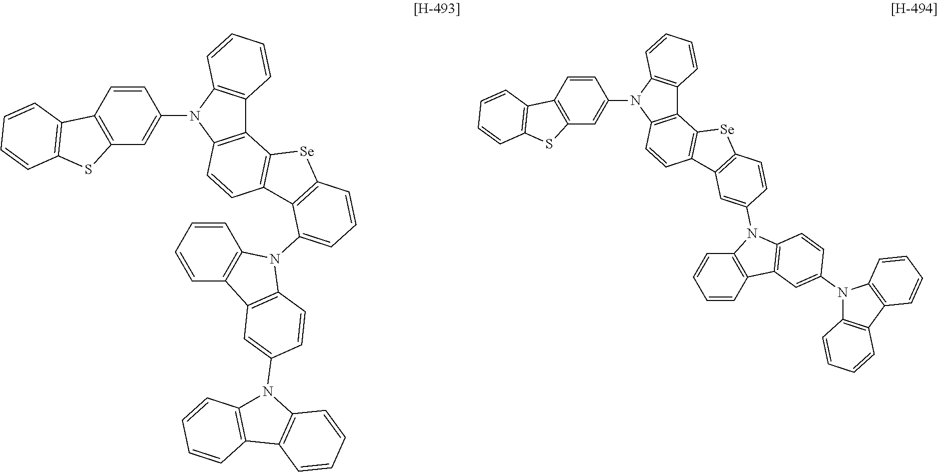 [SMILES: c1ccc2c(c1)sc1cc(-n3c4ccccc4c4c5[se]c6cccc(-n7c8ccccc8c8cc(-n9c%10ccccc%10c%10ccccc%109)ccc87)c6c5ccc43)ccc12 ; c1ccc2c(c1)sc1cc(-n3c4ccccc4c4c5[se]c6ccc(-n7c8ccccc8c8cc(-n9c%10ccccc%10c%10ccccc%109)ccc87)cc6c5ccc43)ccc12]

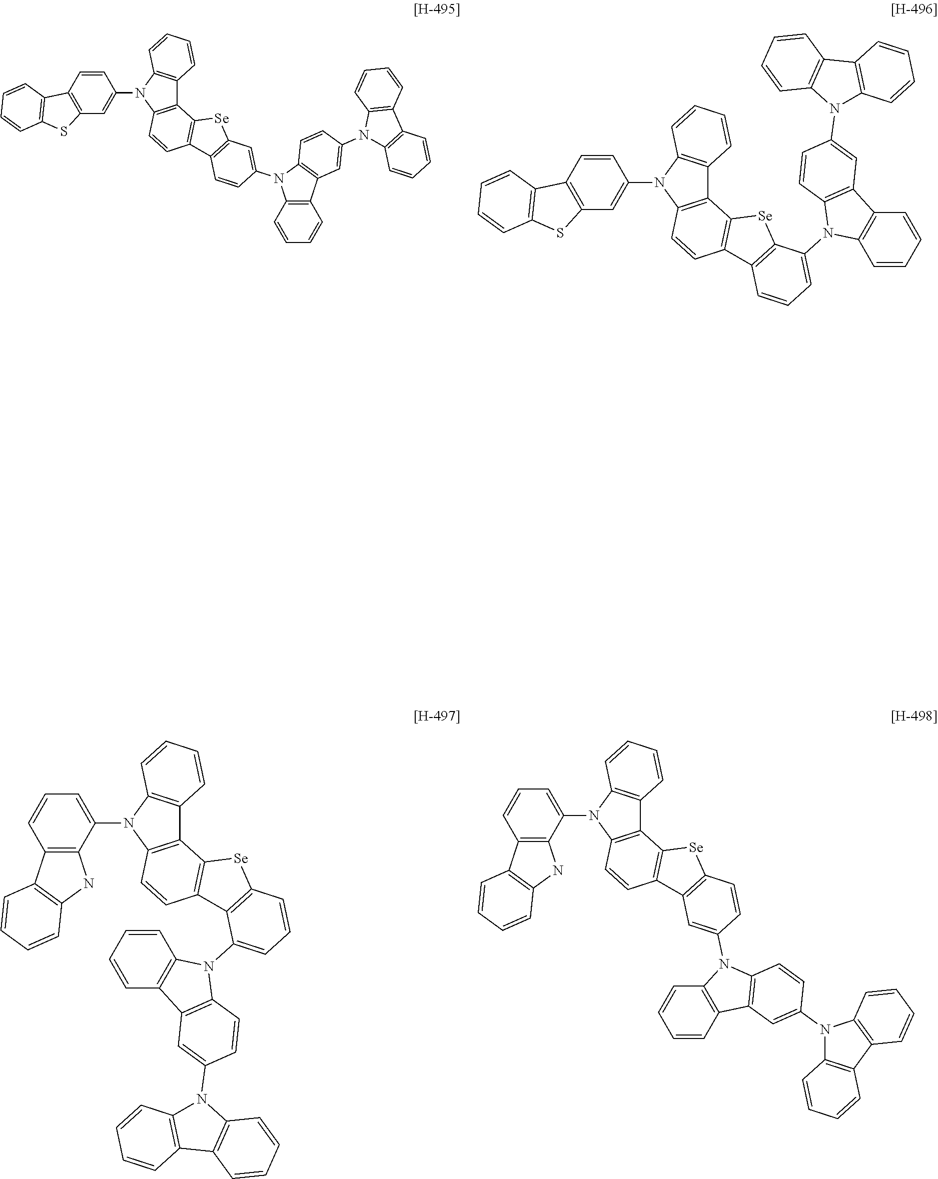 [SMILES: c1ccc2c(c1)sc1cc(-n3c4ccccc4c4c5[se]c6cc(-n7c8ccccc8c8cc(-n9c%10ccccc%10c%10ccccc%109)ccc87)ccc6c5ccc43)ccc12 ; c1ccc2c(c1)sc1cc(-n3c4ccccc4c4c5[se]c6c(-n7c8ccccc8c8cc(-n9c%10ccccc%10c%10ccccc%109)ccc87)cccc6c5ccc43)ccc12 ; c1ccc2c(c1)[nH]c1c(-n3c4ccccc4c4c5[se]c6cccc(-n7c8ccccc8c8cc(-n9c%10ccccc%10c%10ccccc%109)ccc87)c6c5ccc43)cccc12 ; c1ccc2c(c1)[nH]c1c(-n3c4ccccc4c4c5[se]c6ccc(-n7c8ccccc8c8cc(-n9c%10ccccc%10c%10ccccc%109)ccc87)cc6c5ccc43)cccc12]

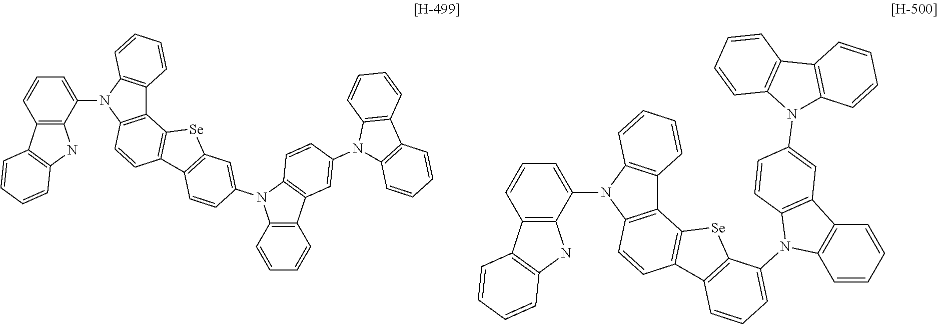 [SMILES: c1ccc2c(c1)[nH]c1c(-n3c4ccccc4c4c5[se]c6cc(-n7c8ccccc8c8cc(-n9c%10ccccc%10c%10ccccc%109)ccc87)ccc6c5ccc43)cccc12 ; c1ccc2c(c1)[nH]c1c(-n3c4ccccc4c4c5[se]c6c(-n7c8ccccc8c8cc(-n9c%10ccccc%10c%10ccccc%109)ccc87)cccc6c5ccc43)cccc12]

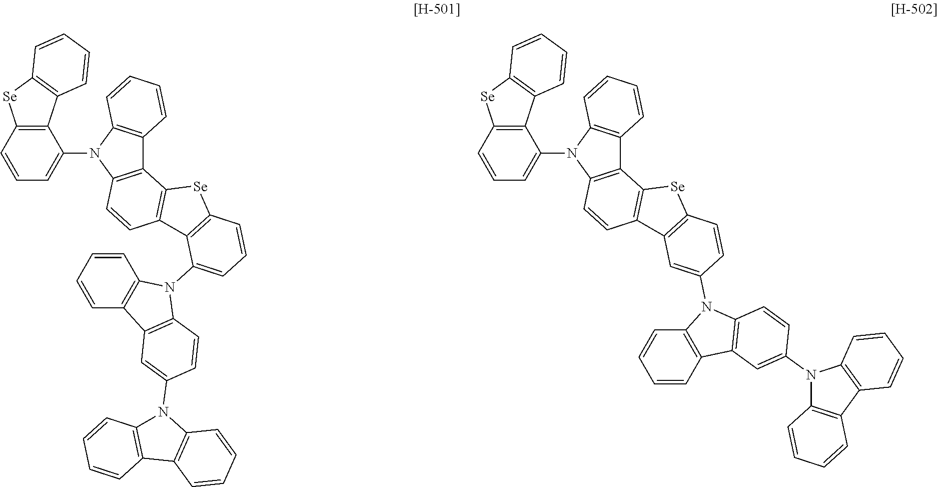 [SMILES: c1ccc2c(c1)[se]c1cccc(-n3c4ccccc4c4c5[se]c6cccc(-n7c8ccccc8c8cc(-n9c%10ccccc%10c%10ccccc%109)ccc87)c6c5ccc43)c12 ; c1ccc2c(c1)[se]c1cccc(-n3c4ccccc4c4c5[se]c6ccc(-n7c8ccccc8c8cc(-n9c%10ccccc%10c%10ccccc%109)ccc87)cc6c5ccc43)c12]

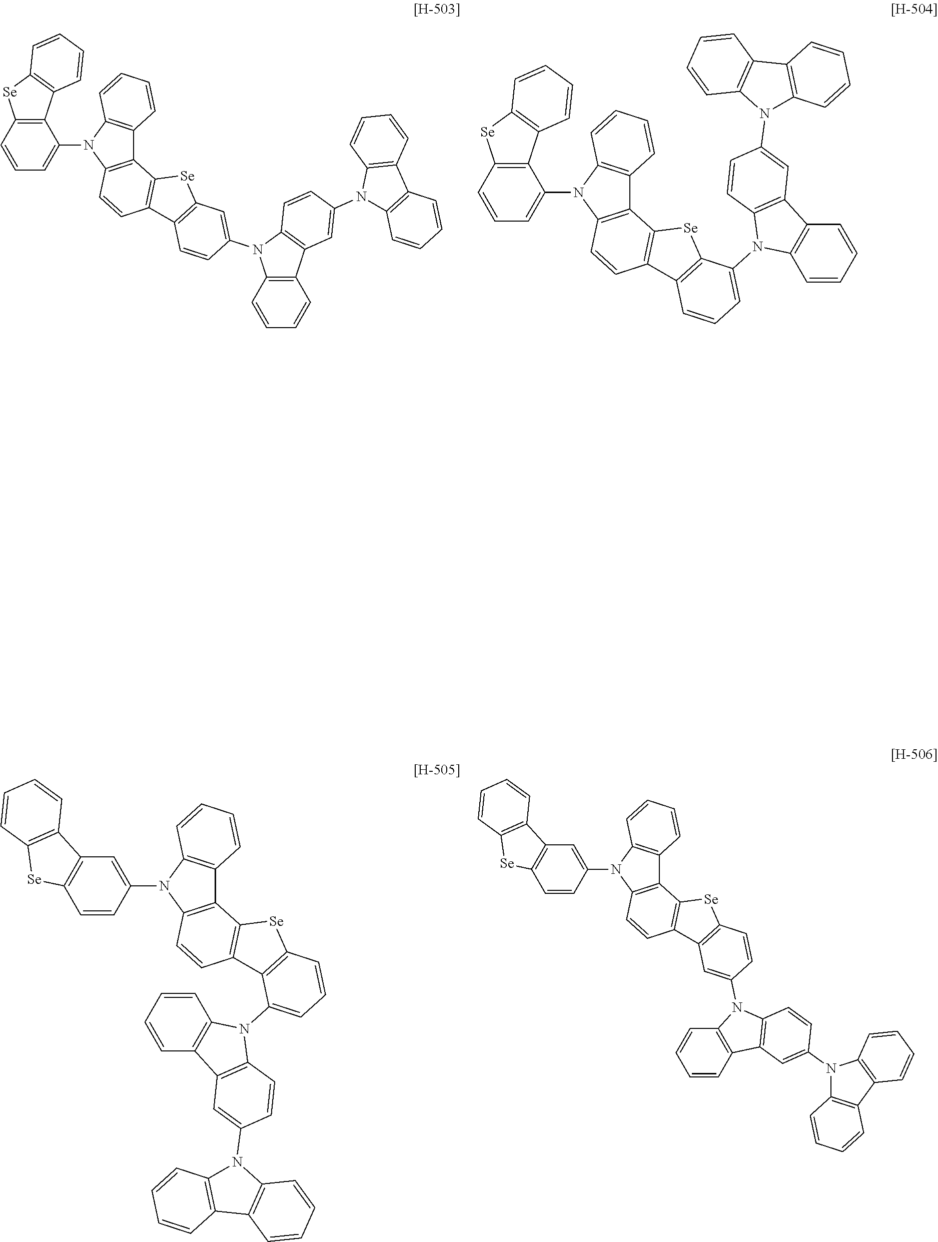

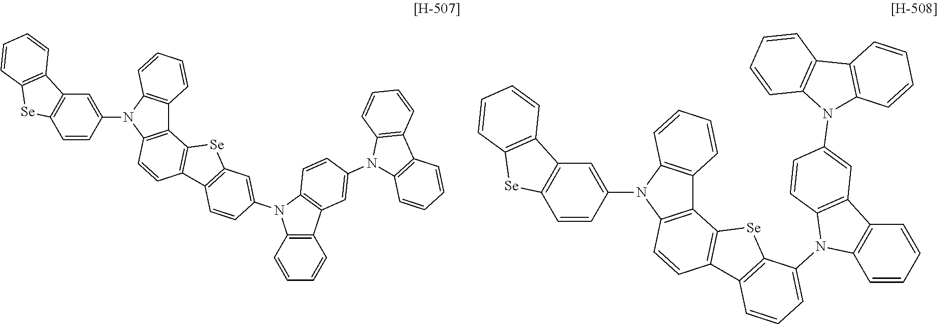 [SMILES: c1ccc2c(c1)[se]c1ccc(-n3c4ccccc4c4c5[se]c6cc(-n7c8ccccc8c8cc(-n9c%10ccccc%10c%10ccccc%109)ccc87)ccc6c5ccc43)cc12 ; c1ccc2c(c1)[se]c1ccc(-n3c4ccccc4c4c5[se]c6c(-n7c8ccccc8c8cc(-n9c%10ccccc%10c%10ccccc%109)ccc87)cccc6c5ccc43)cc12]

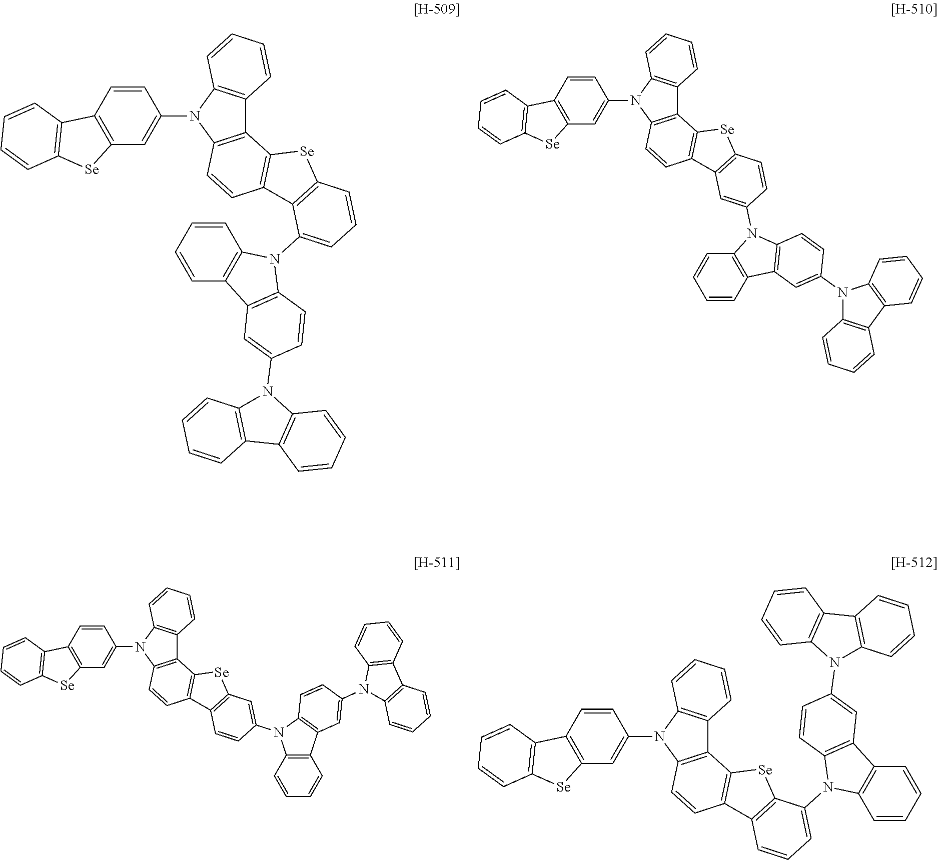 [SMILES: c1ccc2c(c1)[se]c1cc(-n3c4ccccc4c4c5[se]c6cccc(-n7c8ccccc8c8cc(-n9c%10ccccc%10c%10ccccc%109)ccc87)c6c5ccc43)ccc12 ; c1ccc2c(c1)[se]c1cc(-n3c4ccccc4c4c5[se]c6ccc(-n7c8ccccc8c8cc(-n9c%10ccccc%10c%10ccccc%109)ccc87)cc6c5ccc43)ccc12 ; c1ccc2c(c1)[se]c1cc(-n3c4ccccc4c4c5[se]c6cc(-n7c8ccccc8c8cc(-n9c%10ccccc%10c%10ccccc%109)ccc87)ccc6c5ccc43)ccc12 ; c1ccc2c(c1)[se]c1cc(-n3c4ccccc4c4c5[se]c6c(-n7c8ccccc8c8cc(-n9c%10ccccc%10c%10ccccc%109)ccc87)cccc6c5ccc43)ccc12]

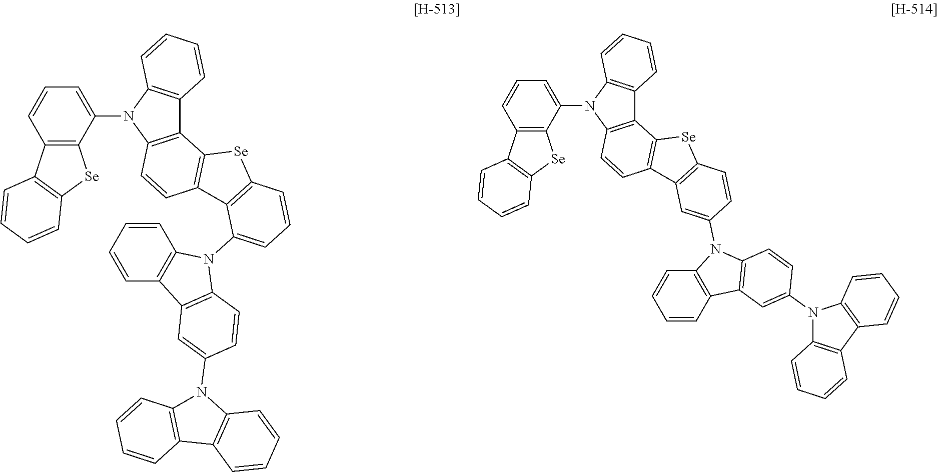 [SMILES: c1ccc2c(c1)[se]c1c(-n3c4ccccc4c4c5[se]c6cccc(-n7c8ccccc8c8cc(-n9c%10ccccc%10c%10ccccc%109)ccc87)c6c5ccc43)cccc12 ; c1ccc2c(c1)[se]c1c(-n3c4ccccc4c4c5[se]c6ccc(-n7c8ccccc8c8cc(-n9c%10ccccc%10c%10ccccc%109)ccc87)cc6c5ccc43)cccc12]

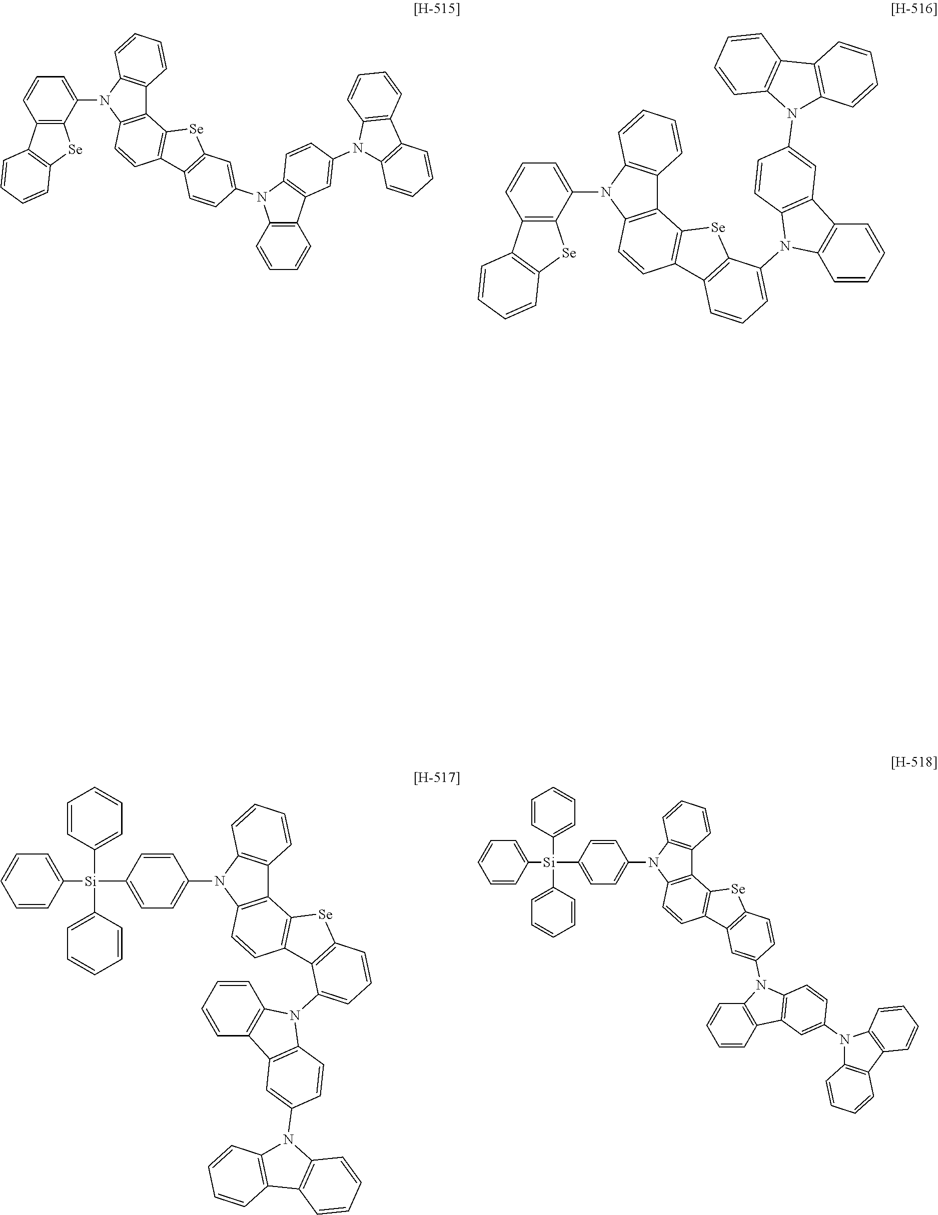 [SMILES: c1ccc2c(c1)[se]c1c(-n3c4ccccc4c4c5[se]c6cc(-n7c8ccccc8c8cc(-n9c%10ccccc%10c%10ccccc%109)ccc87)ccc6c5ccc43)cccc12 ; c1ccc2c(c1)[se]c1c(-n3c4ccccc4c4c5[se]c6c(-n7c8ccccc8c8cc(-n9c%10ccccc%10c%10ccccc%109)ccc87)cccc6c5ccc43)cccc12 ; c1ccc([Si](c2ccccc2)(c2ccccc2)c2ccc(-n3c4ccccc4c4c5[se]c6cccc(-n7c8ccccc8c8cc(-n9c%10ccccc%10c%10ccccc%109)ccc87)c6c5ccc43)cc2)cc1 ; c1ccc([Si](c2ccccc2)(c2ccccc2)c2ccc(-n3c4ccccc4c4c5[se]c6ccc(-n7c8ccccc8c8cc(-n9c%10ccccc%10c%10ccccc%109)ccc87)cc6c5ccc43)cc2)cc1]

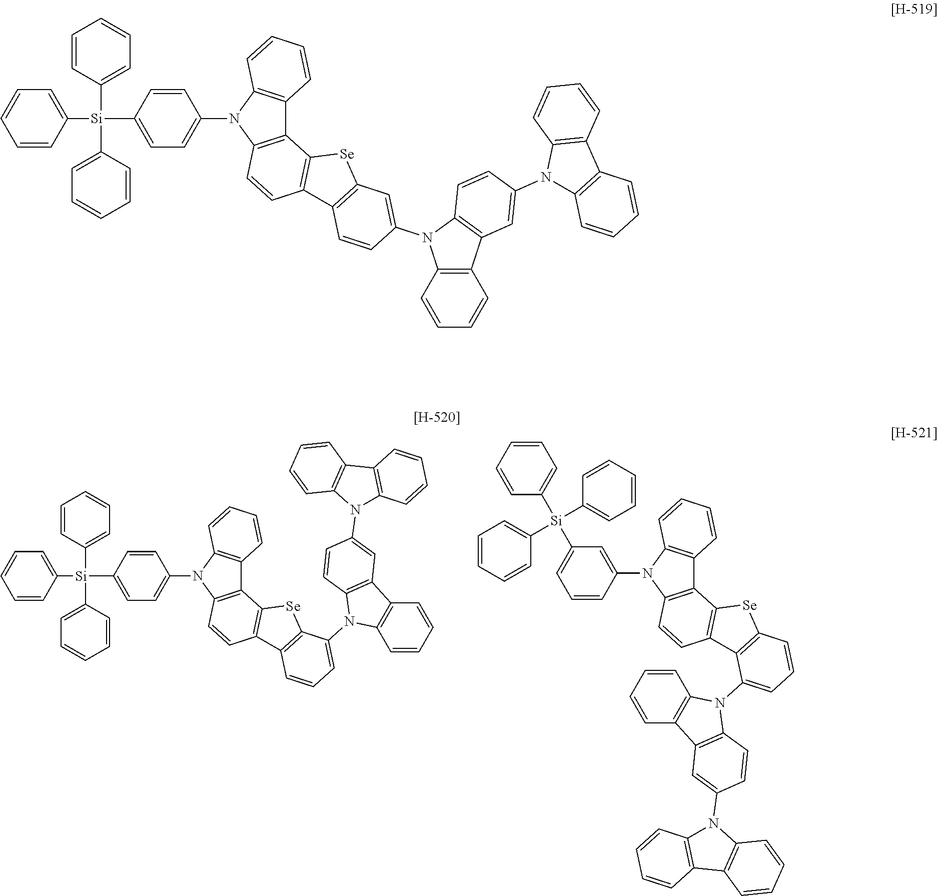 [SMILES: c1ccc([Si](c2ccccc2)(c2ccccc2)c2ccc(-n3c4ccccc4c4c5[se]c6cc(-n7c8ccccc8c8cc(-n9c%10ccccc%10c%10ccccc%109)ccc87)ccc6c5ccc43)cc2)cc1 ; c1ccc([Si](c2ccccc2)(c2ccccc2)c2ccc(-n3c4ccccc4c4c5[se]c6c(-n7c8ccccc8c8cc(-n9c%10ccccc%10c%10ccccc%109)ccc87)cccc6c5ccc43)cc2)cc1 ; c1ccc([Si](c2ccccc2)(c2ccccc2)c2cccc(-n3c4ccccc4c4c5[se]c6cccc(-n7c8ccccc8c8cc(-n9c%10ccccc%10c%10ccccc%109)ccc87)c6c5ccc43)c2)cc1]

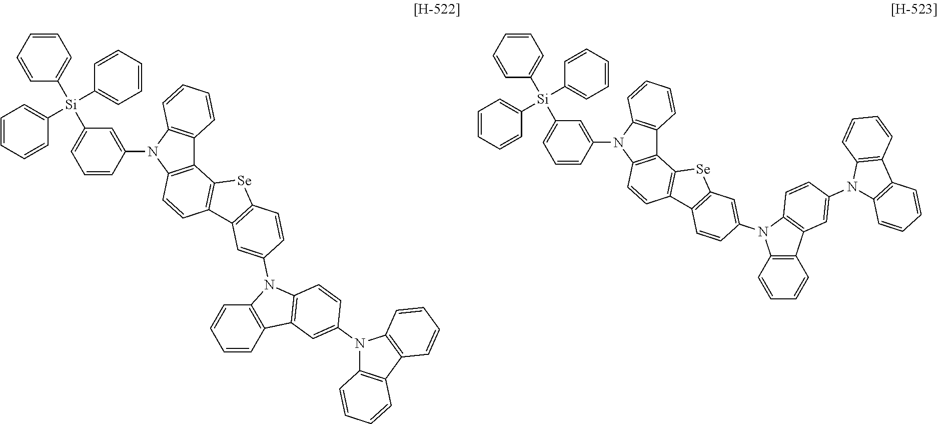 [SMILES: c1ccc([Si](c2ccccc2)(c2ccccc2)c2cccc(-n3c4ccccc4c4c5[se]c6ccc(-n7c8ccccc8c8cc(-n9c%10ccccc%10c%10ccccc%109)ccc87)cc6c5ccc43)c2)cc1 ; c1ccc([Si](c2ccccc2)(c2ccccc2)c2cccc(-n3c4ccccc4c4c5[se]c6cc(-n7c8ccccc8c8cc(-n9c%10ccccc%10c%10ccccc%109)ccc87)ccc6c5ccc43)c2)cc1]

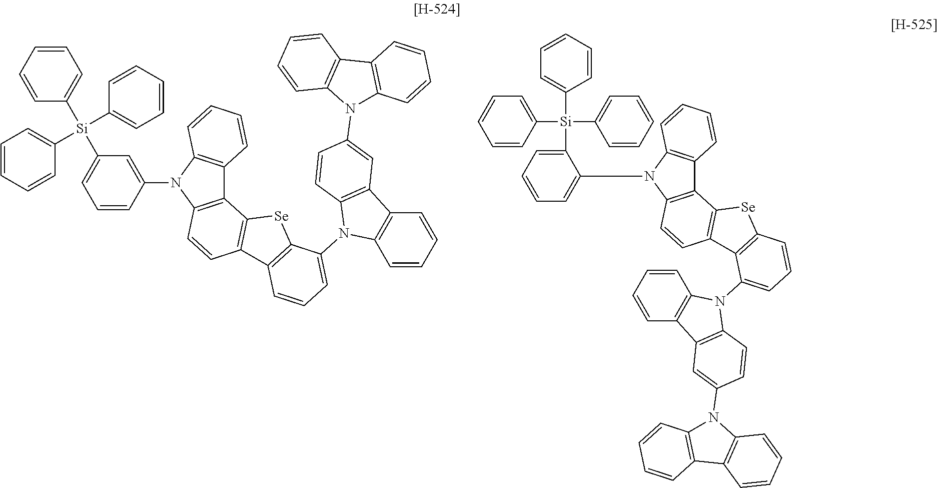 [SMILES: c1ccc([Si](c2ccccc2)(c2ccccc2)c2cccc(-n3c4ccccc4c4c5[se]c6c(-n7c8ccccc8c8cc(-n9c%10ccccc%10c%10ccccc%109)ccc87)cccc6c5ccc43)c2)cc1 ; c1ccc([Si](c2ccccc2)(c2ccccc2)c2ccccc2-n2c3ccccc3c3c4[se]c5cccc(-n6c7ccccc7c7cc(-n8c9ccccc9c9ccccc98)ccc76)c5c4ccc32)cc1]

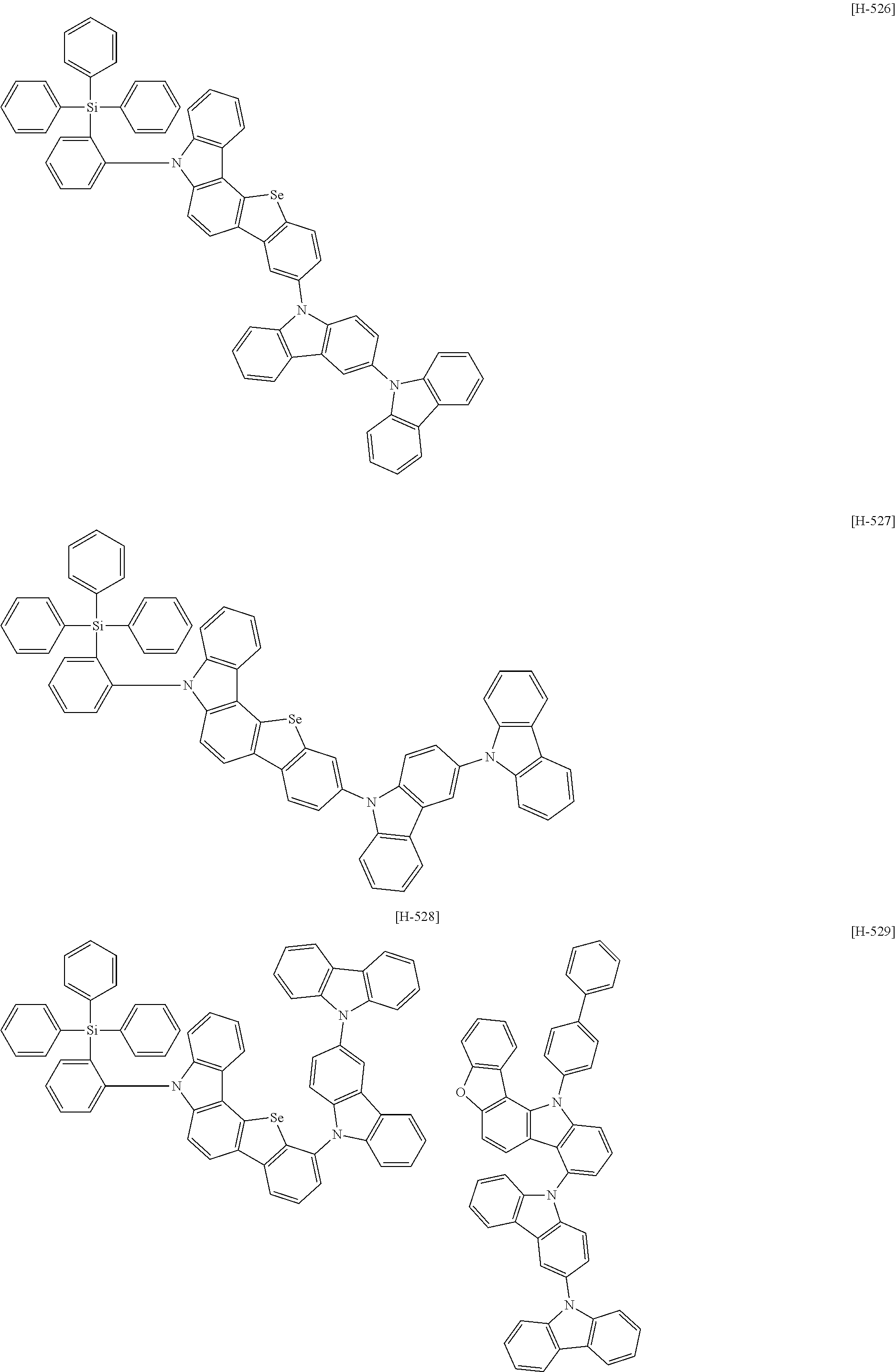 [SMILES: c1ccc([Si](c2ccccc2)(c2ccccc2)c2ccccc2-n2c3ccccc3c3c4[se]c5ccc(-n6c7ccccc7c7cc(-n8c9ccccc9c9ccccc98)ccc76)cc5c4ccc32)cc1 ; c1ccc([Si](c2ccccc2)(c2ccccc2)c2ccccc2-n2c3ccccc3c3c4[se]c5cc(-n6c7ccccc7c7cc(-n8c9ccccc9c9ccccc98)ccc76)ccc5c4ccc32)cc1 ; c1ccc([Si](c2ccccc2)(c2ccccc2)c2ccccc2-n2c3ccccc3c3c4[se]c5c(-n6c7ccccc7c7cc(-n8c9ccccc9c9ccccc98)ccc76)cccc5c4ccc32)cc1 ; c1ccc(-c2ccc(-n3c4cccc(-n5c6ccccc6c6cc(-n7c8ccccc8c8ccccc87)ccc65)c4c4ccc5oc6ccccc6c5c43)cc2)cc1]

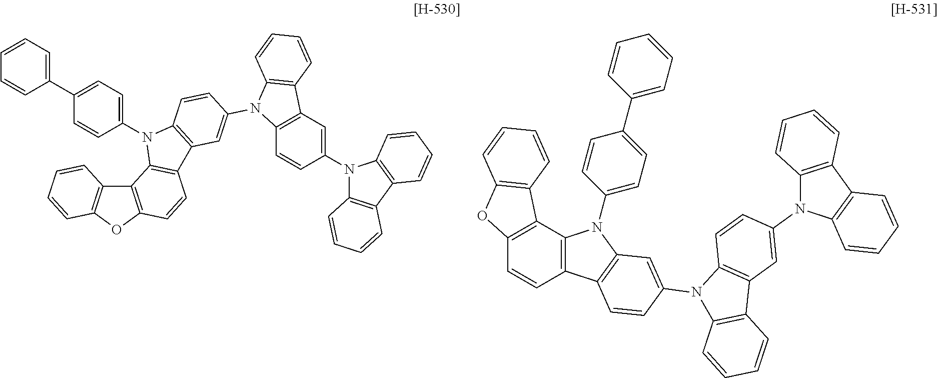 [SMILES: c1ccc(-c2ccc(-n3c4ccc(-n5c6ccccc6c6cc(-n7c8ccccc8c8ccccc87)ccc65)cc4c4ccc5oc6ccccc6c5c43)cc2)cc1 ; c1ccc(-c2ccc(-n3c4cc(-n5c6ccccc6c6cc(-n7c8ccccc8c8ccccc87)ccc65)ccc4c4ccc5oc6ccccc6c5c43)cc2)cc1]

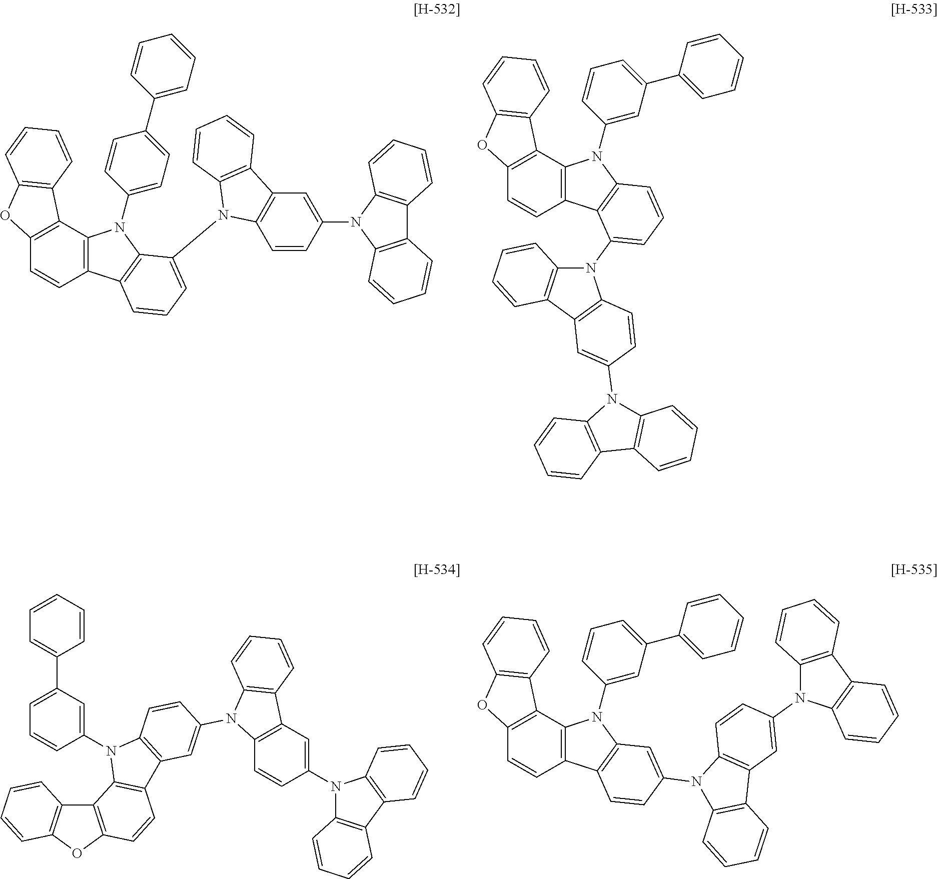 [SMILES: c1ccc(-c2ccc(-n3c4c(-n5c6ccccc6c6cc(-n7c8ccccc8c8ccccc87)ccc65)cccc4c4ccc5oc6ccccc6c5c43)cc2)cc1 ; c1ccc(-c2cccc(-n3c4cccc(-n5c6ccccc6c6cc(-n7c8ccccc8c8ccccc87)ccc65)c4c4ccc5oc6ccccc6c5c43)c2)cc1 ; c1ccc(-c2cccc(-n3c4ccc(-n5c6ccccc6c6cc(-n7c8ccccc8c8ccccc87)ccc65)cc4c4ccc5oc6ccccc6c5c43)c2)cc1 ; c1ccc(-c2cccc(-n3c4cc(-n5c6ccccc6c6cc(-n7c8ccccc8c8ccccc87)ccc65)ccc4c4ccc5oc6ccccc6c5c43)c2)cc1]

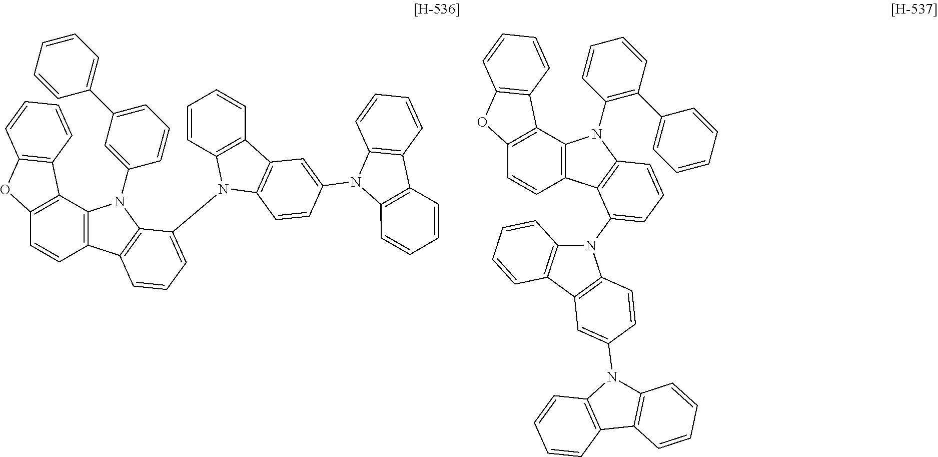 [SMILES: c1ccc(-c2cccc(-n3c4c(-n5c6ccccc6c6cc(-n7c8ccccc8c8ccccc87)ccc65)cccc4c4ccc5oc6ccccc6c5c43)c2)cc1 ; c1ccc(-c2ccccc2-n2c3cccc(-n4c5ccccc5c5cc(-n6c7ccccc7c7ccccc76)ccc54)c3c3ccc4oc5ccccc5c4c32)cc1]

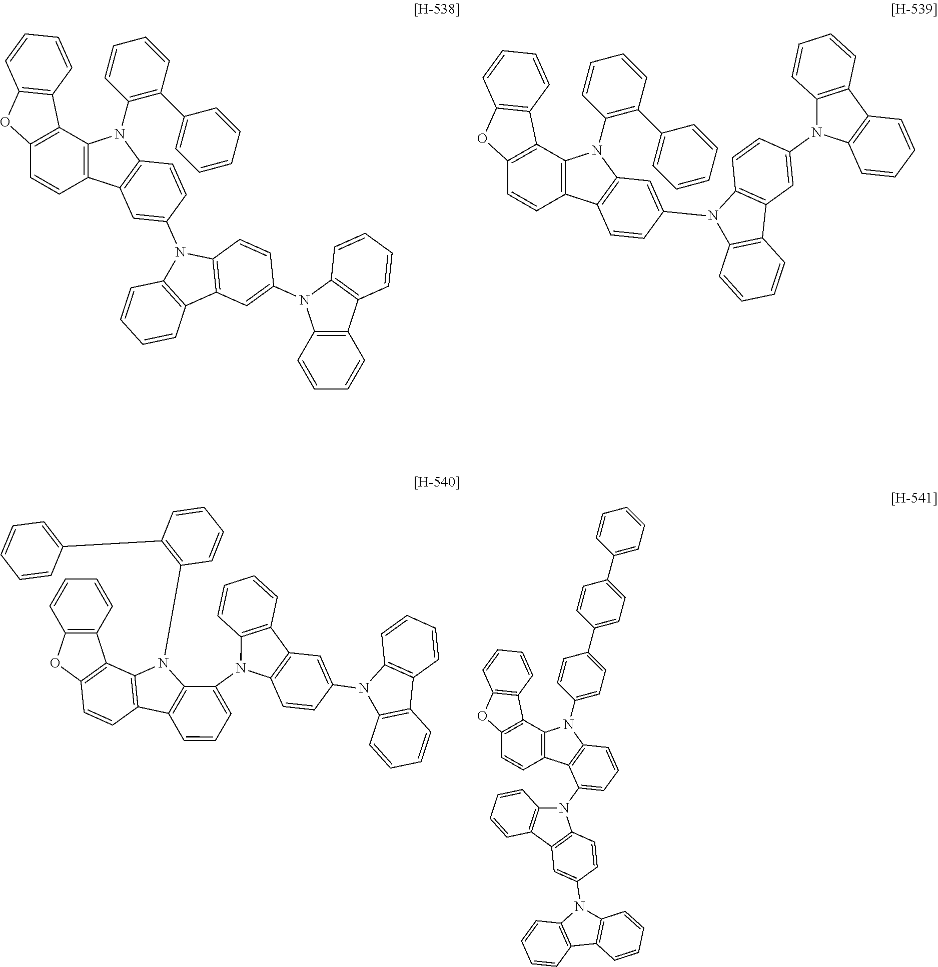 [SMILES: c1ccc(-c2ccccc2-n2c3ccc(-n4c5ccccc5c5cc(-n6c7ccccc7c7ccccc76)ccc54)cc3c3ccc4oc5ccccc5c4c32)cc1 ; c1ccc(-c2ccccc2-n2c3cc(-n4c5ccccc5c5cc(-n6c7ccccc7c7ccccc76)ccc54)ccc3c3ccc4oc5ccccc5c4c32)cc1 ; c1ccc(-c2ccccc2-n2c3c(-n4c5ccccc5c5cc(-n6c7ccccc7c7ccccc76)ccc54)cccc3c3ccc4oc5ccccc5c4c32)cc1 ; c1ccc(-c2ccc(-c3ccc(-n4c5cccc(-n6c7ccccc7c7cc(-n8c9ccccc9c9ccccc98)ccc76)c5c5ccc6oc7ccccc7c6c54)cc3)cc2)cc1]

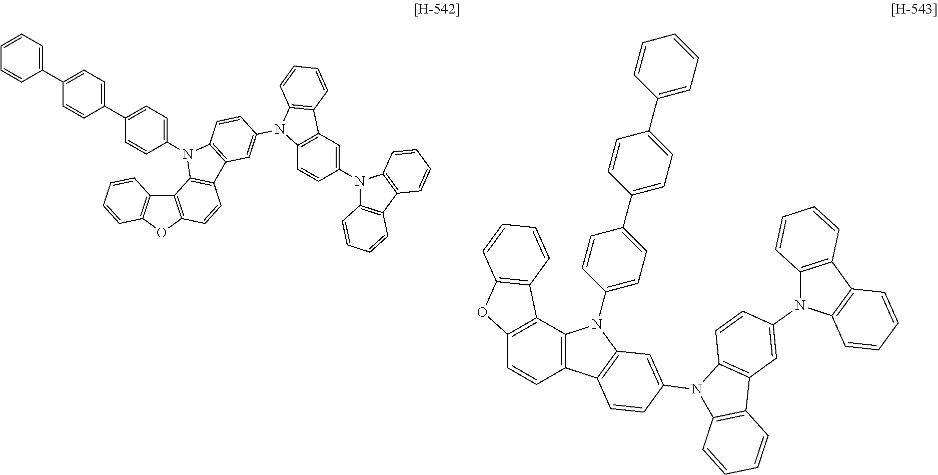 [SMILES: c1ccc(-c2ccc(-c3ccc(-n4c5ccc(-n6c7ccccc7c7cc(-n8c9ccccc9c9ccccc98)ccc76)cc5c5ccc6oc7ccccc7c6c54)cc3)cc2)cc1 ; c1ccc(-c2ccc(-c3ccc(-n4c5cc(-n6c7ccccc7c7cc(-n8c9ccccc9c9ccccc98)ccc76)ccc5c5ccc6oc7ccccc7c6c54)cc3)cc2)cc1]

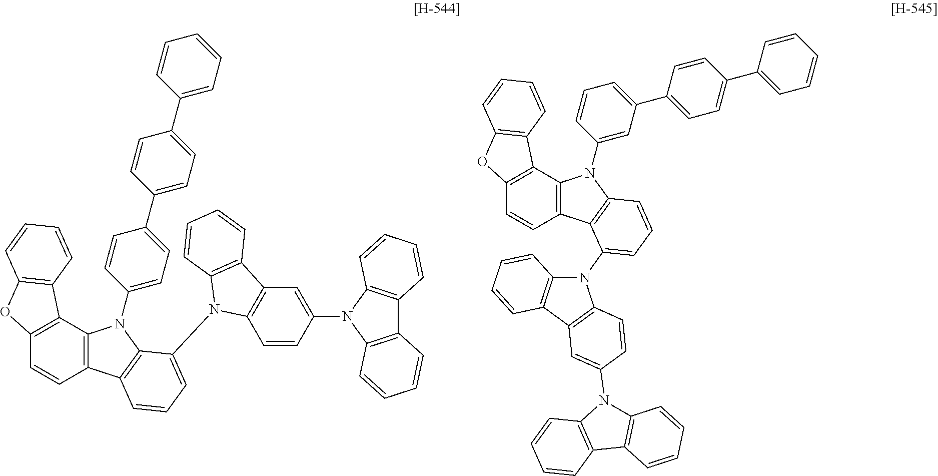 [SMILES: c1ccc(-c2ccc(-c3ccc(-n4c5c(-n6c7ccccc7c7cc(-n8c9ccccc9c9ccccc98)ccc76)cccc5c5ccc6oc7ccccc7c6c54)cc3)cc2)cc1 ; c1ccc(-c2ccc(-c3cccc(-n4c5cccc(-n6c7ccccc7c7cc(-n8c9ccccc9c9ccccc98)ccc76)c5c5ccc6oc7ccccc7c6c54)c3)cc2)cc1]

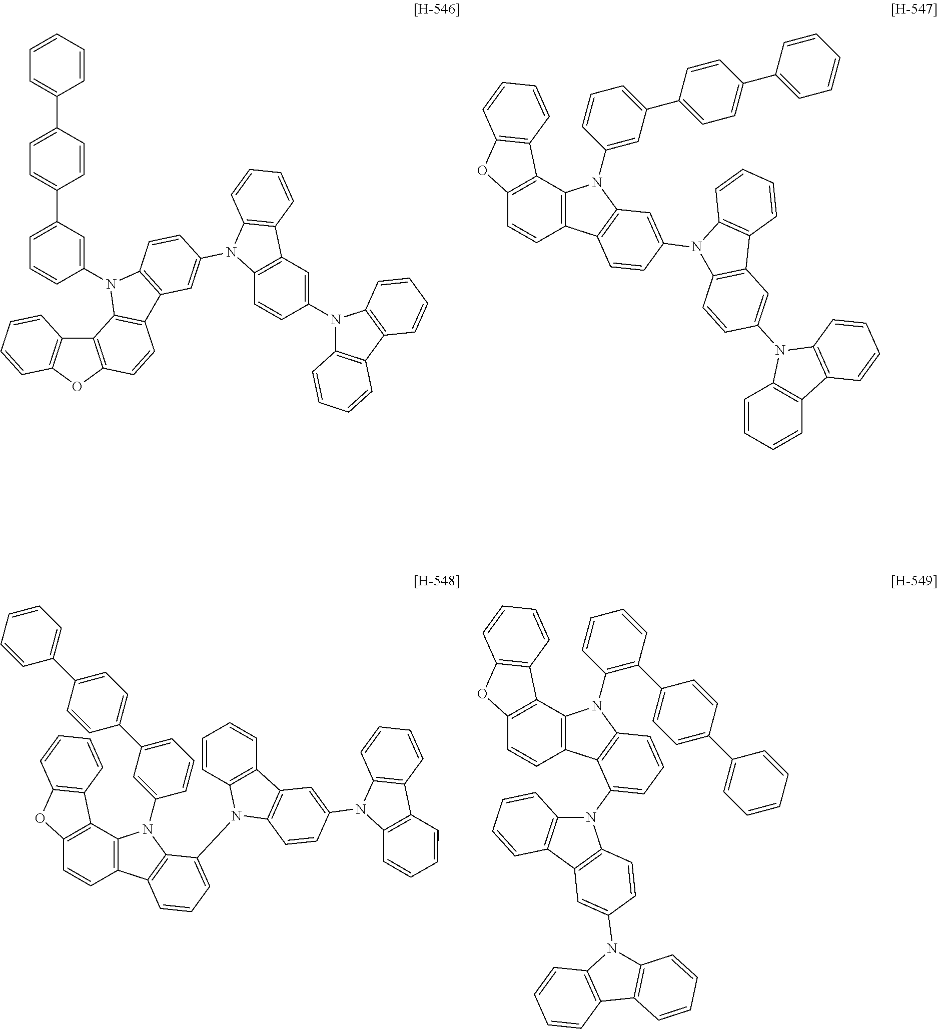 [SMILES: c1ccc(-c2ccc(-c3cccc(-n4c5ccc(-n6c7ccccc7c7cc(-n8c9ccccc9c9ccccc98)ccc76)cc5c5ccc6oc7ccccc7c6c54)c3)cc2)cc1 ; c1ccc(-c2ccc(-c3cccc(-n4c5cc(-n6c7ccccc7c7cc(-n8c9ccccc9c9ccccc98)ccc76)ccc5c5ccc6oc7ccccc7c6c54)c3)cc2)cc1 ; c1ccc(-c2ccc(-c3cccc(-n4c5c(-n6c7ccccc7c7cc(-n8c9ccccc9c9ccccc98)ccc76)cccc5c5ccc6oc7ccccc7c6c54)c3)cc2)cc1 ; c1ccc(-c2ccc(-c3ccccc3-n3c4cccc(-n5c6ccccc6c6cc(-n7c8ccccc8c8ccccc87)ccc65)c4c4ccc5oc6ccccc6c5c43)cc2)cc1]

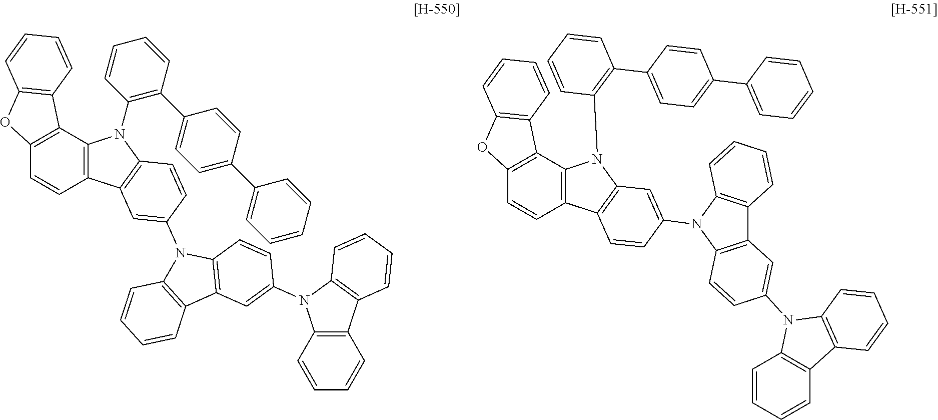 [SMILES: c1ccc(-c2ccc(-c3ccccc3-n3c4ccc(-n5c6ccccc6c6cc(-n7c8ccccc8c8ccccc87)ccc65)cc4c4ccc5oc6ccccc6c5c43)cc2)cc1 ; c1ccc(-c2ccc(-c3ccccc3-n3c4cc(-n5c6ccccc6c6cc(-n7c8ccccc8c8ccccc87)ccc65)ccc4c4ccc5oc6ccccc6c5c43)cc2)cc1]

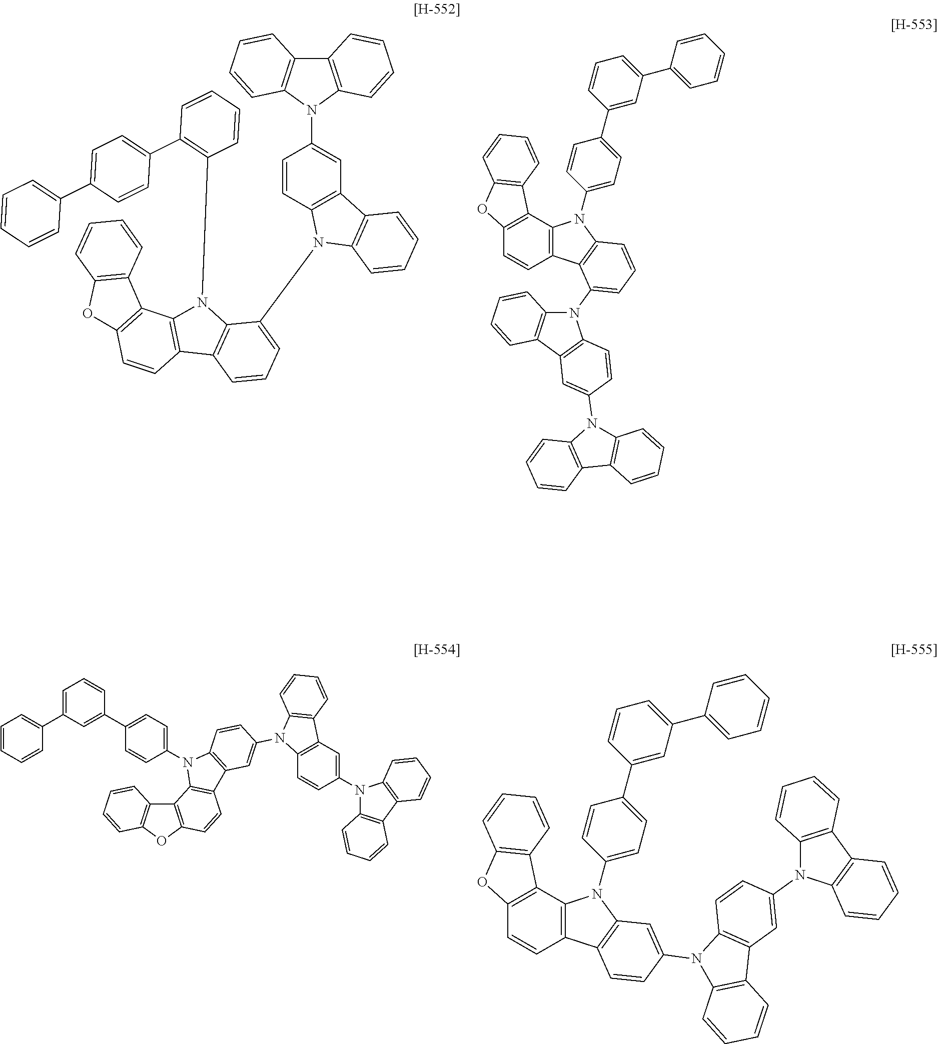 [SMILES: c1ccc(-c2ccc(-c3ccccc3-n3c4c(-n5c6ccccc6c6cc(-n7c8ccccc8c8ccccc87)ccc65)cccc4c4ccc5oc6ccccc6c5c43)cc2)cc1 ; c1ccc(-c2cccc(-c3ccc(-n4c5cccc(-n6c7ccccc7c7cc(-n8c9ccccc9c9ccccc98)ccc76)c5c5ccc6oc7ccccc7c6c54)cc3)c2)cc1 ; c1ccc(-c2cccc(-c3ccc(-n4c5ccc(-n6c7ccccc7c7cc(-n8c9ccccc9c9ccccc98)ccc76)cc5c5ccc6oc7ccccc7c6c54)cc3)c2)cc1 ; c1ccc(-c2cccc(-c3ccc(-n4c5cc(-n6c7ccccc7c7cc(-n8c9ccccc9c9ccccc98)ccc76)ccc5c5ccc6oc7ccccc7c6c54)cc3)c2)cc1]

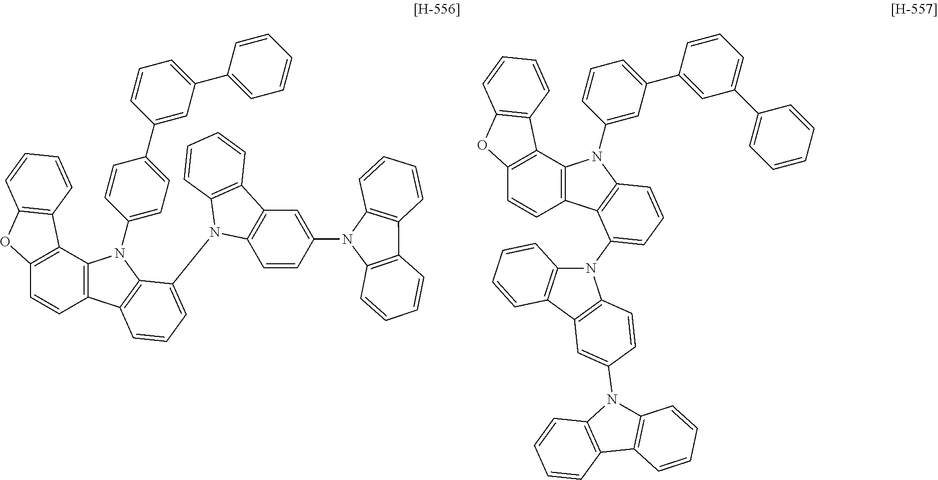 [SMILES: c1ccc(-c2cccc(-c3ccc(-n4c5c(-n6c7ccccc7c7cc(-n8c9ccccc9c9ccccc98)ccc76)cccc5c5ccc6oc7ccccc7c6c54)cc3)c2)cc1 ; c1ccc(-c2cccc(-c3cccc(-n4c5cccc(-n6c7ccccc7c7cc(-n8c9ccccc9c9ccccc98)ccc76)c5c5ccc6oc7ccccc7c6c54)c3)c2)cc1]

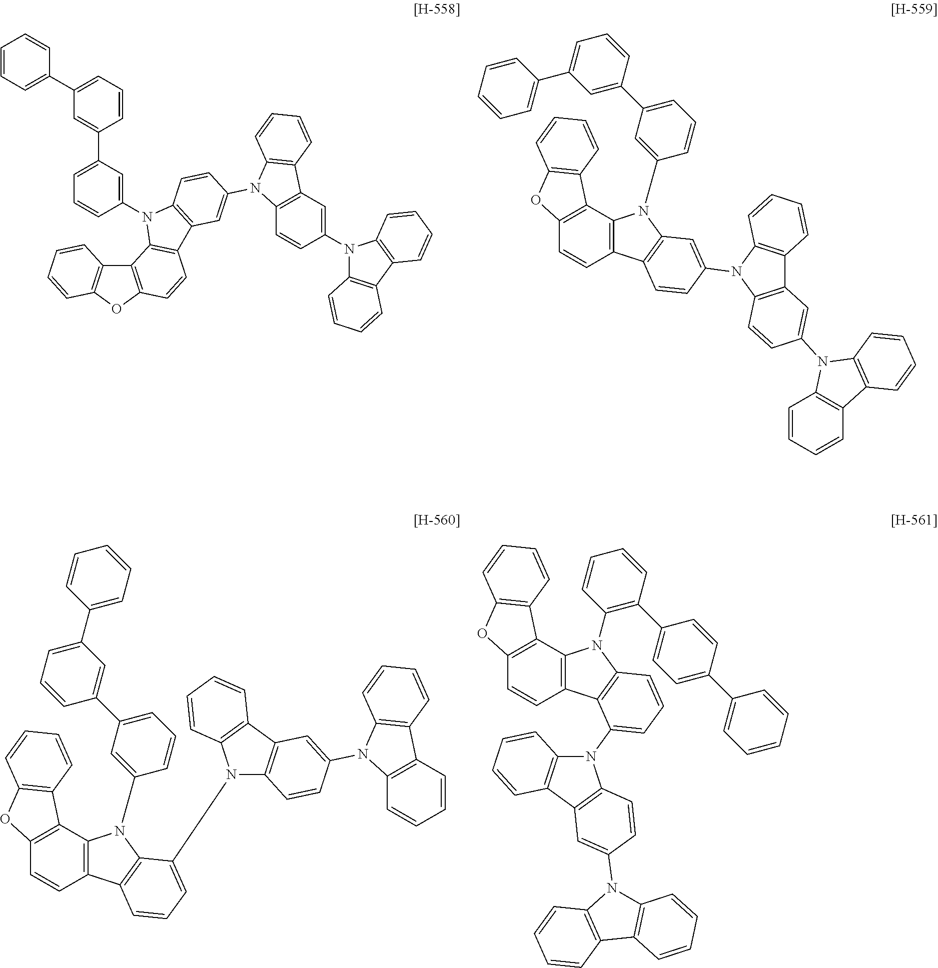 [SMILES: c1ccc(-c2cccc(-c3cccc(-n4c5ccc(-n6c7ccccc7c7cc(-n8c9ccccc9c9ccccc98)ccc76)cc5c5ccc6oc7ccccc7c6c54)c3)c2)cc1 ; c1ccc(-c2cccc(-c3cccc(-n4c5cc(-n6c7ccccc7c7cc(-n8c9ccccc9c9ccccc98)ccc76)ccc5c5ccc6oc7ccccc7c6c54)c3)c2)cc1 ; c1ccc(-c2cccc(-c3cccc(-n4c5c(-n6c7ccccc7c7cc(-n8c9ccccc9c9ccccc98)ccc76)cccc5c5ccc6oc7ccccc7c6c54)c3)c2)cc1 ; c1ccc(-c2ccc(-c3ccccc3-n3c4cccc(-n5c6ccccc6c6cc(-n7c8ccccc8c8ccccc87)ccc65)c4c4ccc5oc6ccccc6c5c43)cc2)cc1]

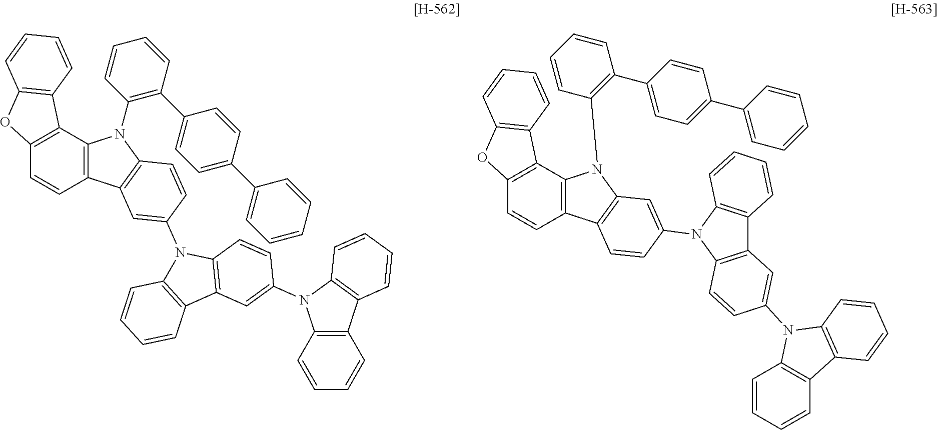 [SMILES: c1ccc(-c2ccc(-c3ccccc3-n3c4ccc(-n5c6ccccc6c6cc(-n7c8ccccc8c8ccccc87)ccc65)cc4c4ccc5oc6ccccc6c5c43)cc2)cc1 ; c1ccc(-c2ccc(-c3ccccc3-n3c4cc(-n5c6ccccc6c6cc(-n7c8ccccc8c8ccccc87)ccc65)ccc4c4ccc5oc6ccccc6c5c43)cc2)cc1]

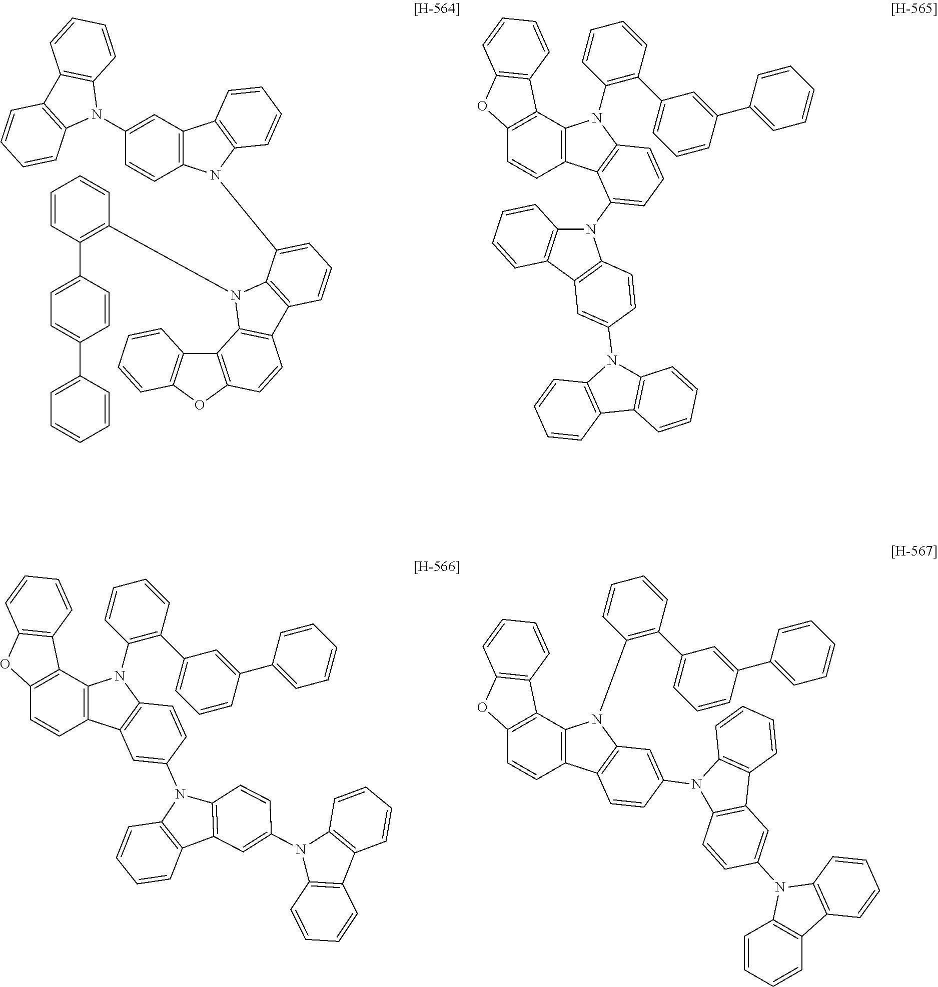 [SMILES: c1ccc(-c2ccc(-c3ccccc3-n3c4c(-n5c6ccccc6c6cc(-n7c8ccccc8c8ccccc87)ccc65)cccc4c4ccc5oc6ccccc6c5c43)cc2)cc1 ; c1ccc(-c2cccc(-c3ccccc3-n3c4cccc(-n5c6ccccc6c6cc(-n7c8ccccc8c8ccccc87)ccc65)c4c4ccc5oc6ccccc6c5c43)c2)cc1 ; c1ccc(-c2cccc(-c3ccccc3-n3c4ccc(-n5c6ccccc6c6cc(-n7c8ccccc8c8ccccc87)ccc65)cc4c4ccc5oc6ccccc6c5c43)c2)cc1 ; c1ccc(-c2cccc(-c3ccccc3-n3c4cc(-n5c6ccccc6c6cc(-n7c8ccccc8c8ccccc87)ccc65)ccc4c4ccc5oc6ccccc6c5c43)c2)cc1]

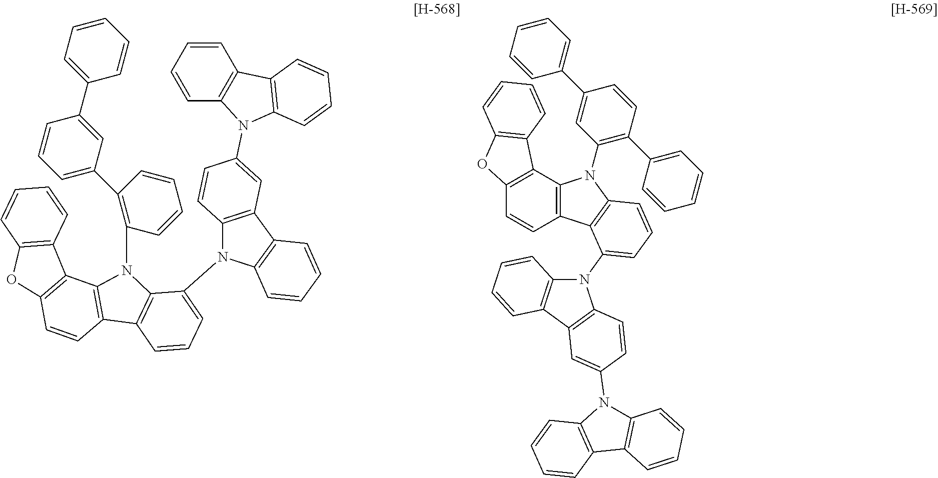 [SMILES: c1ccc(-c2cccc(-c3ccccc3-n3c4c(-n5c6ccccc6c6cc(-n7c8ccccc8c8ccccc87)ccc65)cccc4c4ccc5oc6ccccc6c5c43)c2)cc1 ; c1ccc(-c2ccc(-c3ccccc3)c(-n3c4cccc(-n5c6ccccc6c6cc(-n7c8ccccc8c8ccccc87)ccc65)c4c4ccc5oc6ccccc6c5c43)c2)cc1]

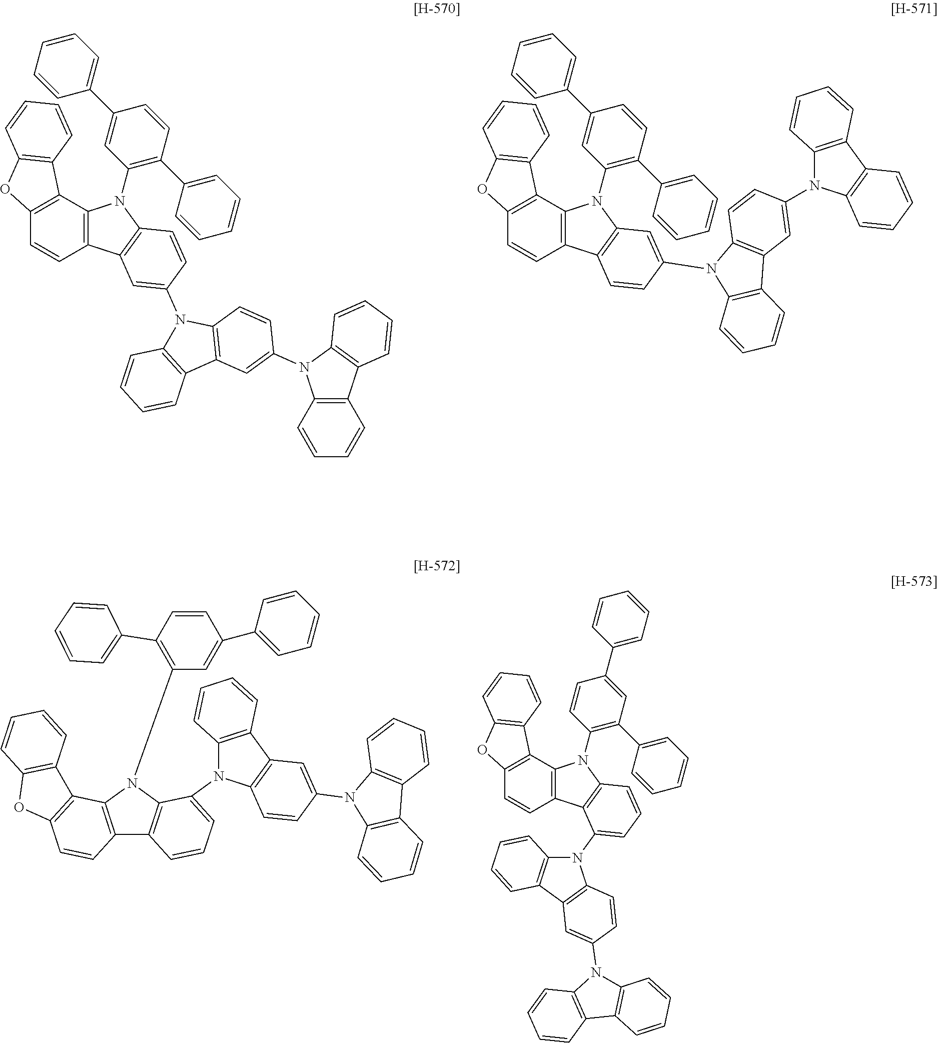 [SMILES: c1ccc(-c2ccc(-c3ccccc3)c(-n3c4ccc(-n5c6ccccc6c6cc(-n7c8ccccc8c8ccccc87)ccc65)cc4c4ccc5oc6ccccc6c5c43)c2)cc1 ; c1ccc(-c2ccc(-c3ccccc3)c(-n3c4cc(-n5c6ccccc6c6cc(-n7c8ccccc8c8ccccc87)ccc65)ccc4c4ccc5oc6ccccc6c5c43)c2)cc1 ; c1ccc(-c2ccc(-c3ccccc3)c(-n3c4c(-n5c6ccccc6c6cc(-n7c8ccccc8c8ccccc87)ccc65)cccc4c4ccc5oc6ccccc6c5c43)c2)cc1 ; c1ccc(-c2ccc(-n3c4cccc(-n5c6ccccc6c6cc(-n7c8ccccc8c8ccccc87)ccc65)c4c4ccc5oc6ccccc6c5c43)c(-c3ccccc3)c2)cc1]

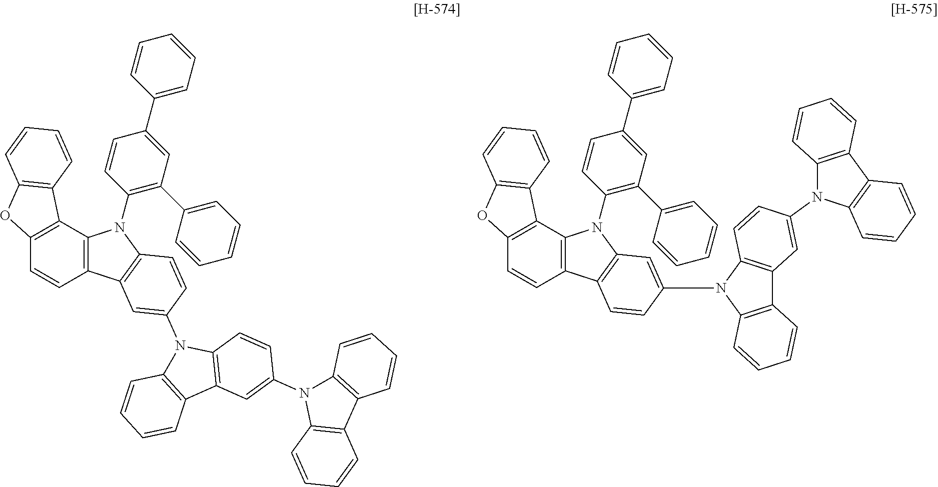 [SMILES: c1ccc(-c2ccc(-n3c4ccc(-n5c6ccccc6c6cc(-n7c8ccccc8c8ccccc87)ccc65)cc4c4ccc5oc6ccccc6c5c43)c(-c3ccccc3)c2)cc1 ; c1ccc(-c2ccc(-n3c4cc(-n5c6ccccc6c6cc(-n7c8ccccc8c8ccccc87)ccc65)ccc4c4ccc5oc6ccccc6c5c43)c(-c3ccccc3)c2)cc1]

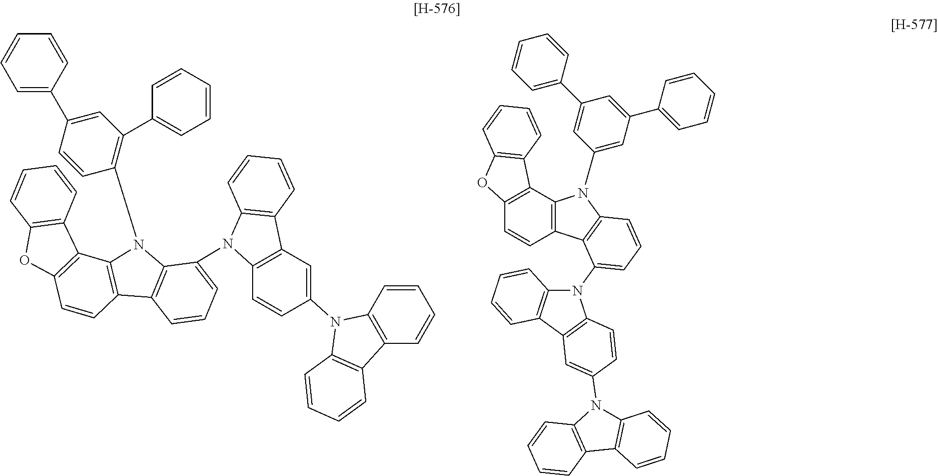 [SMILES: c1ccc(-c2ccc(-n3c4c(-n5c6ccccc6c6cc(-n7c8ccccc8c8ccccc87)ccc65)cccc4c4ccc5oc6ccccc6c5c43)c(-c3ccccc3)c2)cc1 ; c1ccc(-c2cc(-c3ccccc3)cc(-n3c4cccc(-n5c6ccccc6c6cc(-n7c8ccccc8c8ccccc87)ccc65)c4c4ccc5oc6ccccc6c5c43)c2)cc1]

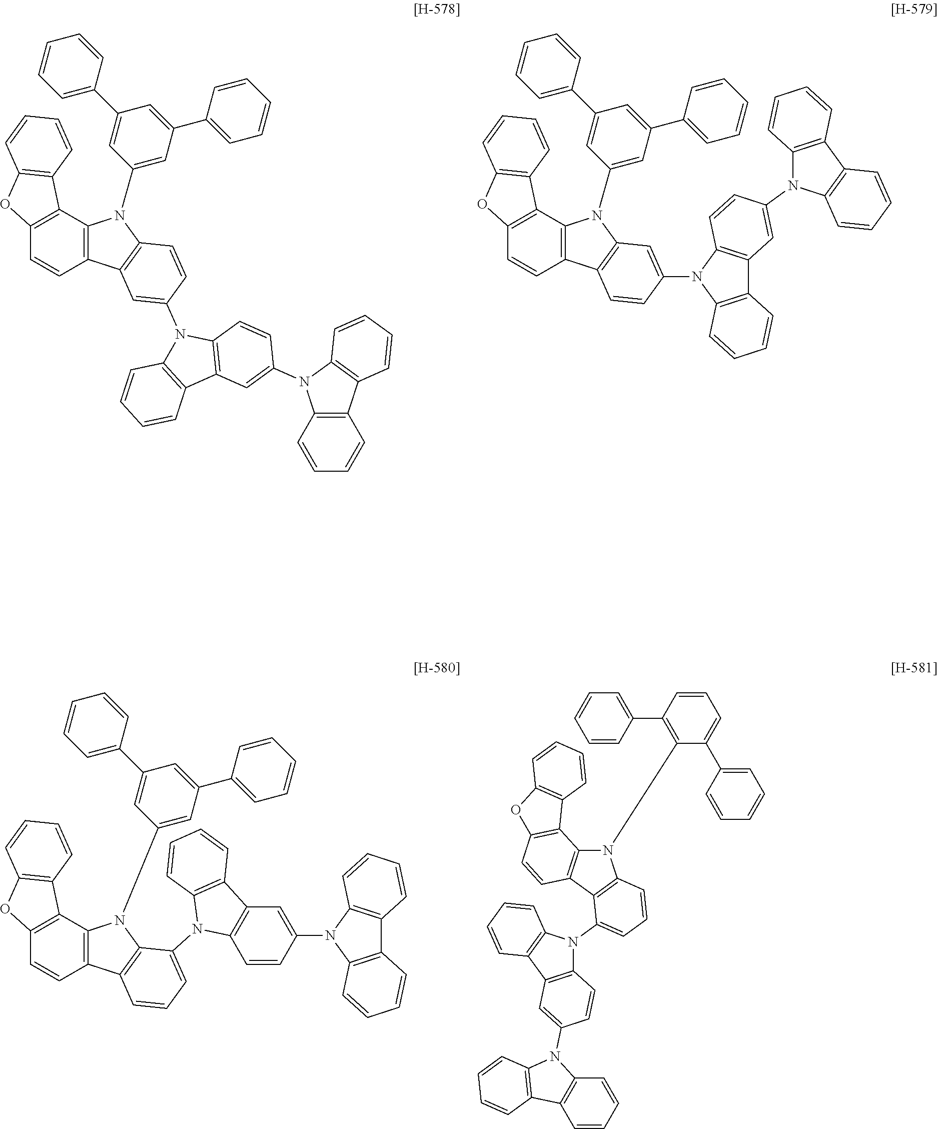 [SMILES: c1ccc(-c2cc(-c3ccccc3)cc(-n3c4ccc(-n5c6ccccc6c6cc(-n7c8ccccc8c8ccccc87)ccc65)cc4c4ccc5oc6ccccc6c5c43)c2)cc1 ; c1ccc(-c2cc(-c3ccccc3)cc(-n3c4cc(-n5c6ccccc6c6cc(-n7c8ccccc8c8ccccc87)ccc65)ccc4c4ccc5oc6ccccc6c5c43)c2)cc1 ; c1ccc(-c2cc(-c3ccccc3)cc(-n3c4c(-n5c6ccccc6c6cc(-n7c8ccccc8c8ccccc87)ccc65)cccc4c4ccc5oc6ccccc6c5c43)c2)cc1 ; c1ccc(-c2cccc(-c3ccccc3)c2-n2c3cccc(-n4c5ccccc5c5cc(-n6c7ccccc7c7ccccc76)ccc54)c3c3ccc4oc5ccccc5c4c32)cc1]

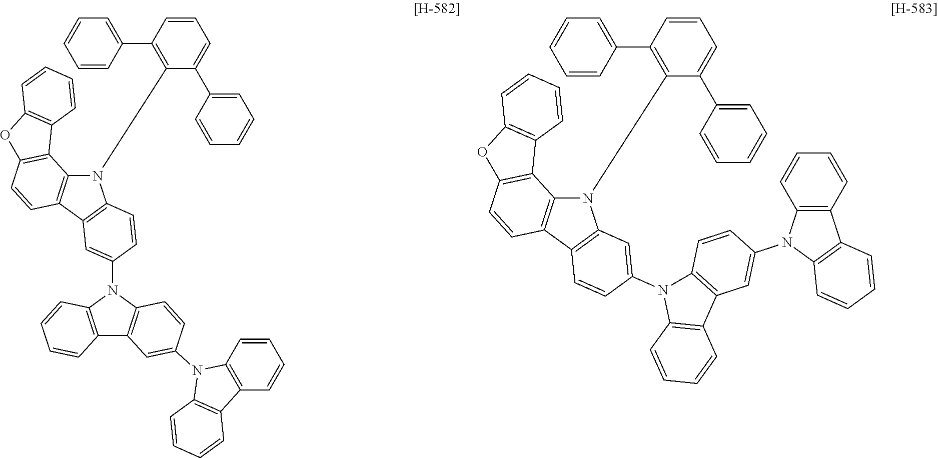 [SMILES: c1ccc(-c2cccc(-c3ccccc3)c2-n2c3ccc(-n4c5ccccc5c5cc(-n6c7ccccc7c7ccccc76)ccc54)cc3c3ccc4oc5ccccc5c4c32)cc1 ; c1ccc(-c2cccc(-c3ccccc3)c2-n2c3cc(-n4c5ccccc5c5cc(-n6c7ccccc7c7ccccc76)ccc54)ccc3c3ccc4oc5ccccc5c4c32)cc1]

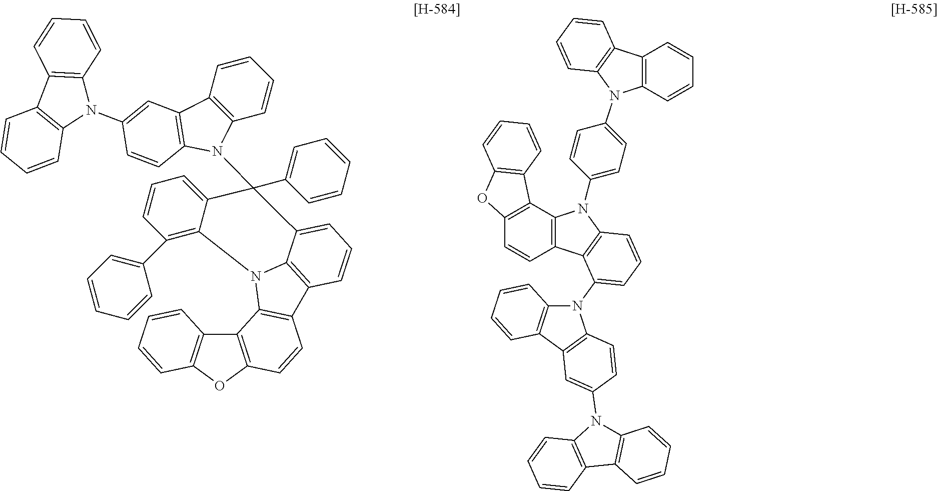 [SMILES: c1ccc(-c2cccc3c2-n2c4c(cccc4c4ccc5oc6ccccc6c5c42)C3(c2ccccc2)n2c3ccccc3c3cc(-n4c5ccccc5c5ccccc54)ccc32)cc1 ; c1ccc2c(c1)oc1ccc3c4c(-n5c6ccccc6c6cc(-n7c8ccccc8c8ccccc87)ccc65)cccc4n(-c4ccc(-n5c6ccccc6c6ccccc65)cc4)c3c12]

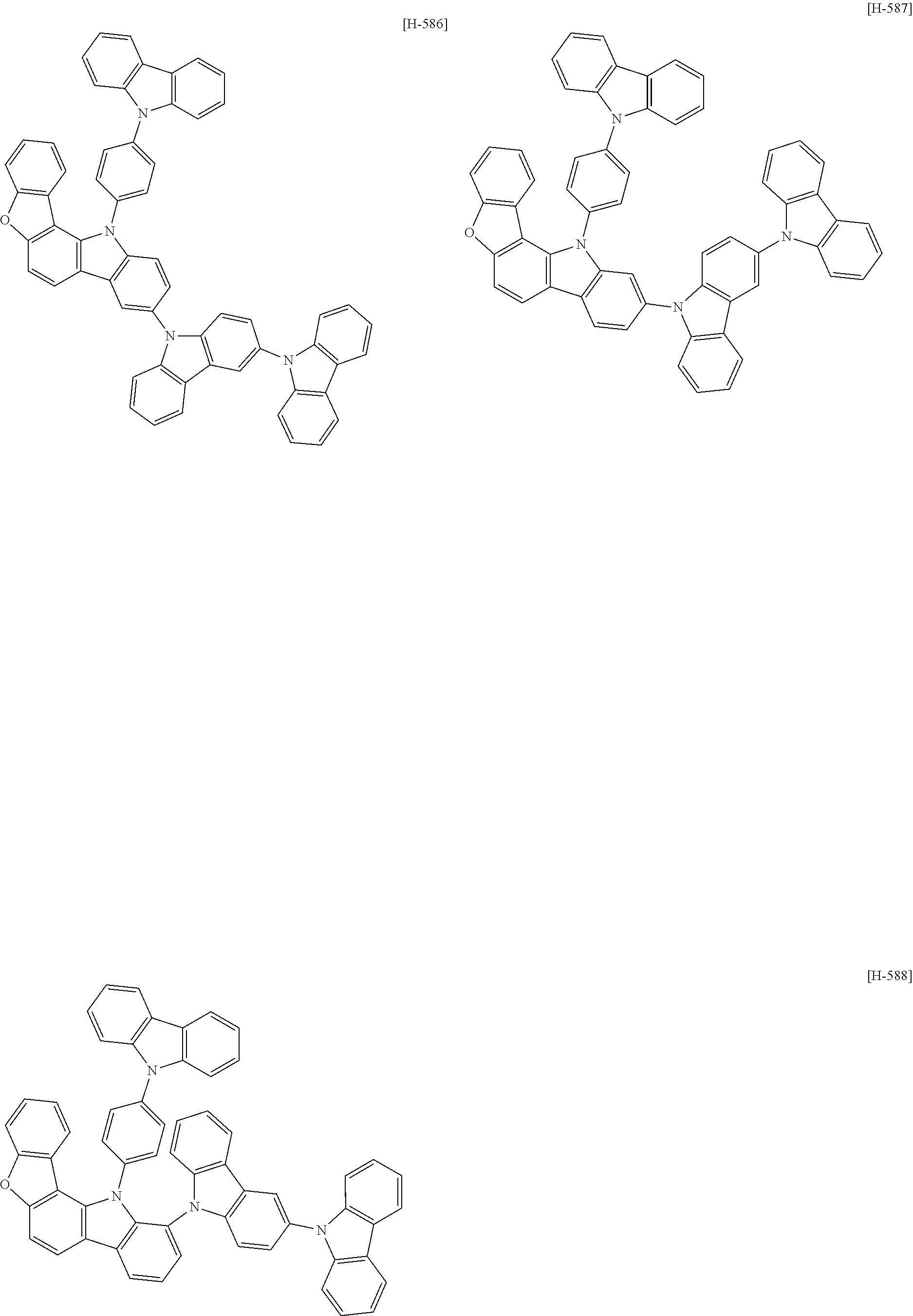 [SMILES: c1ccc2c(c1)oc1ccc3c4cc(-n5c6ccccc6c6cc(-n7c8ccccc8c8ccccc87)ccc65)ccc4n(-c4ccc(-n5c6ccccc6c6ccccc65)cc4)c3c12 ; c1ccc2c(c1)oc1ccc3c4ccc(-n5c6ccccc6c6cc(-n7c8ccccc8c8ccccc87)ccc65)cc4n(-c4ccc(-n5c6ccccc6c6ccccc65)cc4)c3c12 ; c1ccc2c(c1)oc1ccc3c4cccc(-n5c6ccccc6c6cc(-n7c8ccccc8c8ccccc87)ccc65)c4n(-c4ccc(-n5c6ccccc6c6ccccc65)cc4)c3c12]

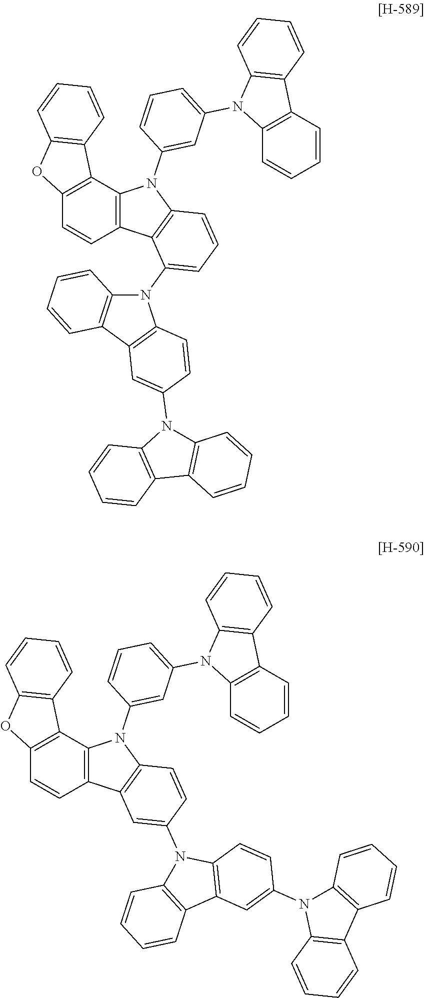 [SMILES: c1cc(-n2c3ccccc3c3ccccc32)cc(-n2c3cccc(-n4c5ccccc5c5cc(-n6c7ccccc7c7ccccc76)ccc54)c3c3ccc4oc5ccccc5c4c32)c1 ; c1cc(-n2c3ccccc3c3ccccc32)cc(-n2c3ccc(-n4c5ccccc5c5cc(-n6c7ccccc7c7ccccc76)ccc54)cc3c3ccc4oc5ccccc5c4c32)c1]

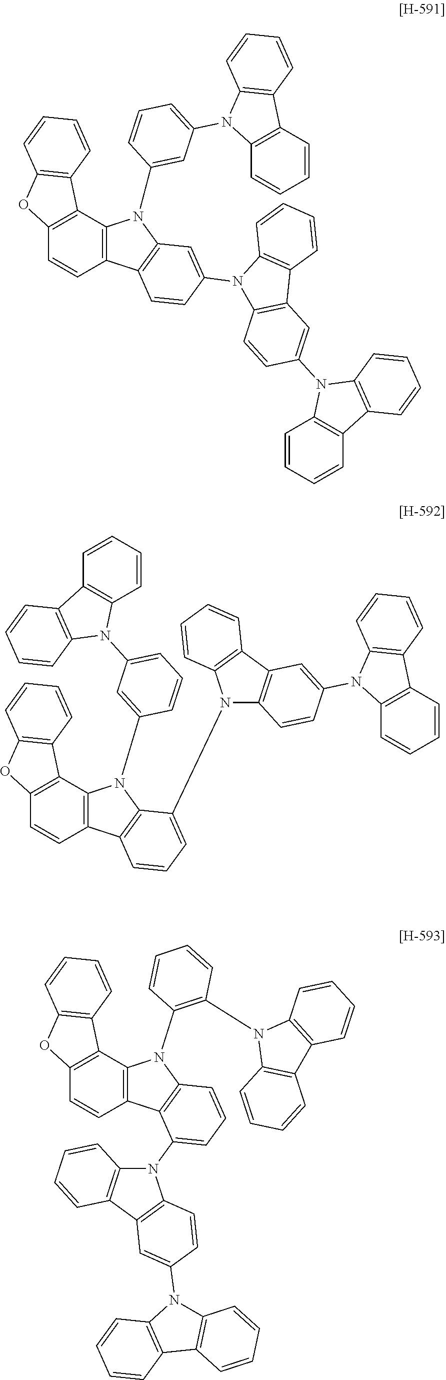 [SMILES: c1cc(-n2c3ccccc3c3ccccc32)cc(-n2c3cc(-n4c5ccccc5c5cc(-n6c7ccccc7c7ccccc76)ccc54)ccc3c3ccc4oc5ccccc5c4c32)c1 ; c1cc(-n2c3ccccc3c3ccccc32)cc(-n2c3c(-n4c5ccccc5c5cc(-n6c7ccccc7c7ccccc76)ccc54)cccc3c3ccc4oc5ccccc5c4c32)c1 ; c1ccc(-n2c3cccc(-n4c5ccccc5c5cc(-n6c7ccccc7c7ccccc76)ccc54)c3c3ccc4oc5ccccc5c4c32)c(-n2c3ccccc3c3ccccc32)c1]

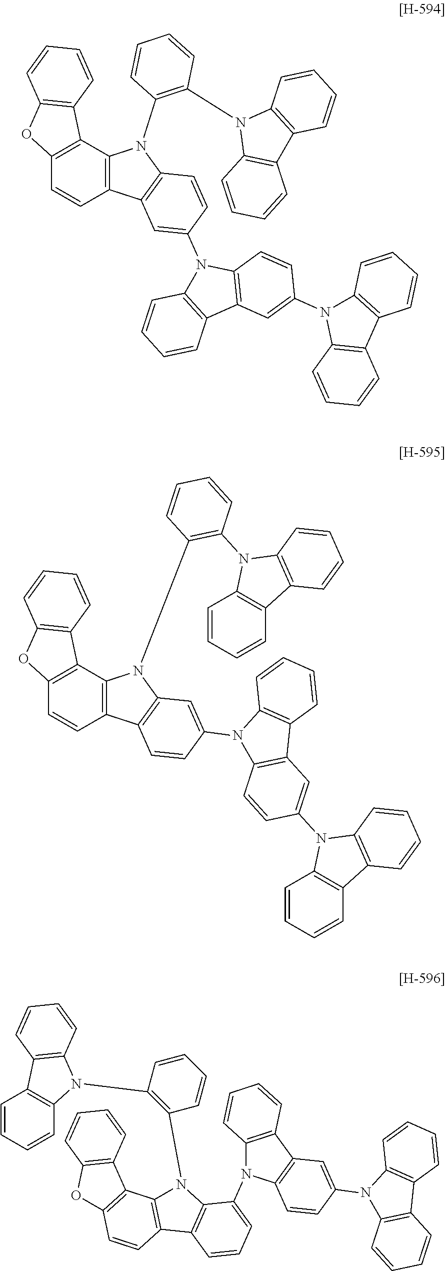 [SMILES: c1ccc(-n2c3ccc(-n4c5ccccc5c5cc(-n6c7ccccc7c7ccccc76)ccc54)cc3c3ccc4oc5ccccc5c4c32)c(-n2c3ccccc3c3ccccc32)c1 ; c1ccc(-n2c3cc(-n4c5ccccc5c5cc(-n6c7ccccc7c7ccccc76)ccc54)ccc3c3ccc4oc5ccccc5c4c32)c(-n2c3ccccc3c3ccccc32)c1 ; c1ccc(-n2c3c(-n4c5ccccc5c5cc(-n6c7ccccc7c7ccccc76)ccc54)cccc3c3ccc4oc5ccccc5c4c32)c(-n2c3ccccc3c3ccccc32)c1]

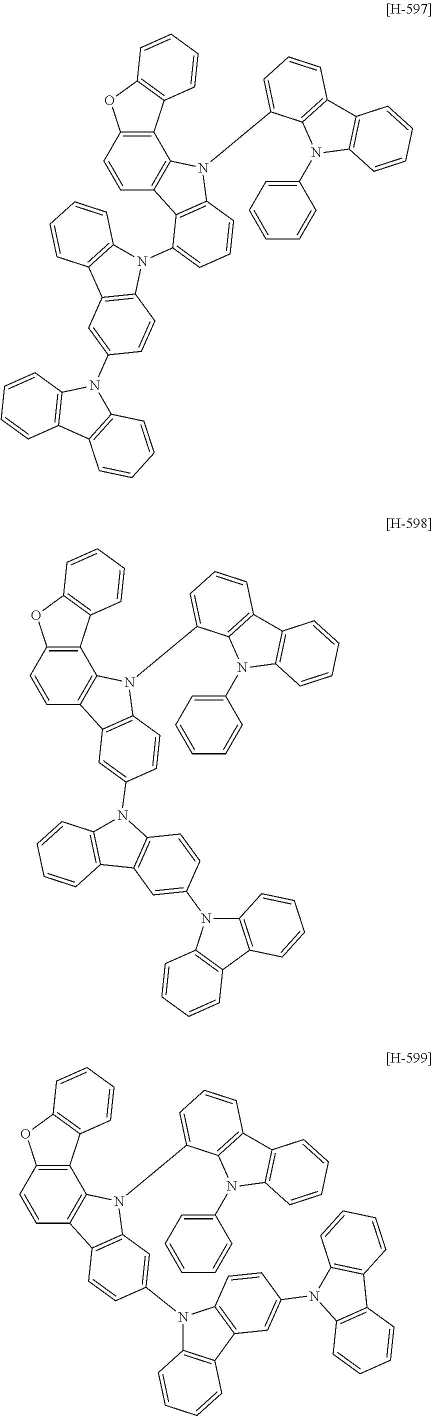 [SMILES: c1ccc(-n2c3ccccc3c3cccc(-n4c5cccc(-n6c7ccccc7c7cc(-n8c9ccccc9c9ccccc98)ccc76)c5c5ccc6oc7ccccc7c6c54)c32)cc1 ; c1ccc(-n2c3ccccc3c3cccc(-n4c5ccc(-n6c7ccccc7c7cc(-n8c9ccccc9c9ccccc98)ccc76)cc5c5ccc6oc7ccccc7c6c54)c32)cc1 ; c1ccc(-n2c3ccccc3c3cccc(-n4c5cc(-n6c7ccccc7c7cc(-n8c9ccccc9c9ccccc98)ccc76)ccc5c5ccc6oc7ccccc7c6c54)c32)cc1]

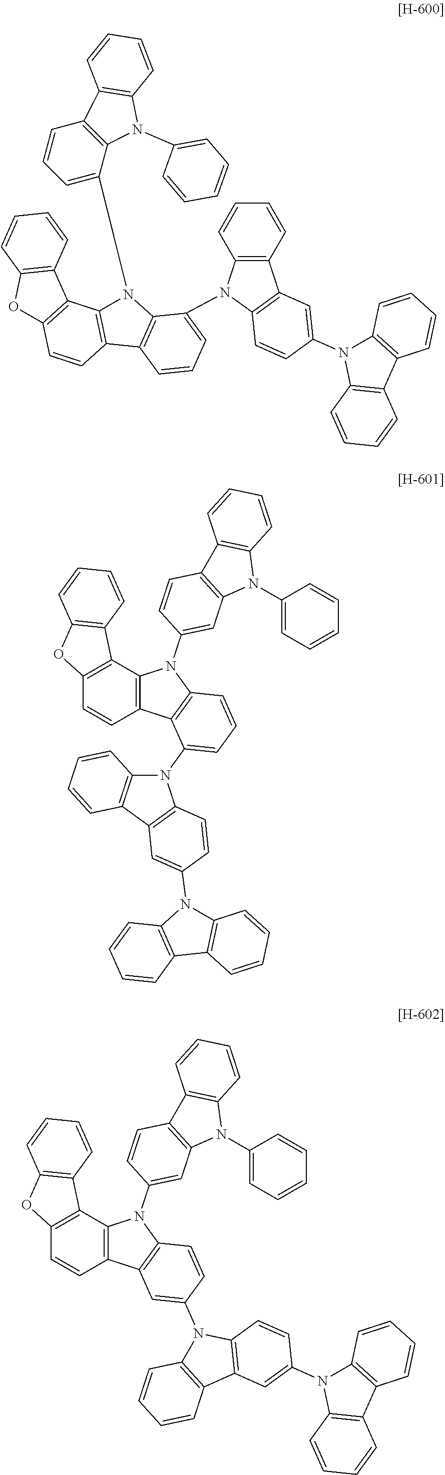 [SMILES: c1ccc(-n2c3ccccc3c3cccc(-n4c5c(-n6c7ccccc7c7cc(-n8c9ccccc9c9ccccc98)ccc76)cccc5c5ccc6oc7ccccc7c6c54)c32)cc1 ; c1ccc(-n2c3ccccc3c3ccc(-n4c5cccc(-n6c7ccccc7c7cc(-n8c9ccccc9c9ccccc98)ccc76)c5c5ccc6oc7ccccc7c6c54)cc32)cc1 ; c1ccc(-n2c3ccccc3c3ccc(-n4c5ccc(-n6c7ccccc7c7cc(-n8c9ccccc9c9ccccc98)ccc76)cc5c5ccc6oc7ccccc7c6c54)cc32)cc1]

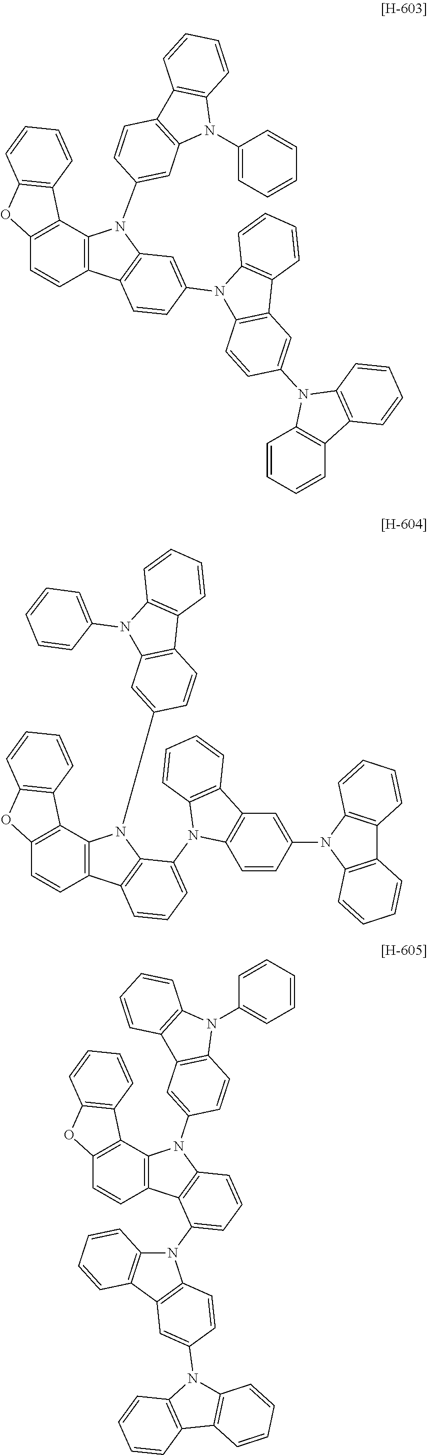 [SMILES: c1ccc(-n2c3ccccc3c3ccc(-n4c5cc(-n6c7ccccc7c7cc(-n8c9ccccc9c9ccccc98)ccc76)ccc5c5ccc6oc7ccccc7c6c54)cc32)cc1 ; c1ccc(-n2c3ccccc3c3ccc(-n4c5c(-n6c7ccccc7c7cc(-n8c9ccccc9c9ccccc98)ccc76)cccc5c5ccc6oc7ccccc7c6c54)cc32)cc1 ; c1ccc(-n2c3ccccc3c3cc(-n4c5cccc(-n6c7ccccc7c7cc(-n8c9ccccc9c9ccccc98)ccc76)c5c5ccc6oc7ccccc7c6c54)ccc32)cc1]

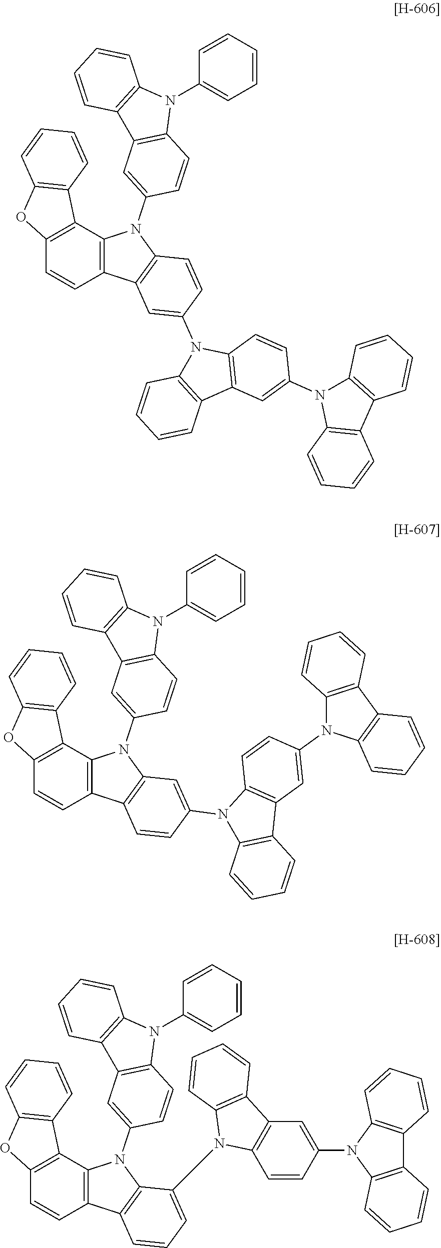 [SMILES: c1ccc(-n2c3ccccc3c3cc(-n4c5ccc(-n6c7ccccc7c7cc(-n8c9ccccc9c9ccccc98)ccc76)cc5c5ccc6oc7ccccc7c6c54)ccc32)cc1 ; c1ccc(-n2c3ccccc3c3cc(-n4c5cc(-n6c7ccccc7c7cc(-n8c9ccccc9c9ccccc98)ccc76)ccc5c5ccc6oc7ccccc7c6c54)ccc32)cc1 ; c1ccc(-n2c3ccccc3c3cc(-n4c5c(-n6c7ccccc7c7cc(-n8c9ccccc9c9ccccc98)ccc76)cccc5c5ccc6oc7ccccc7c6c54)ccc32)cc1]

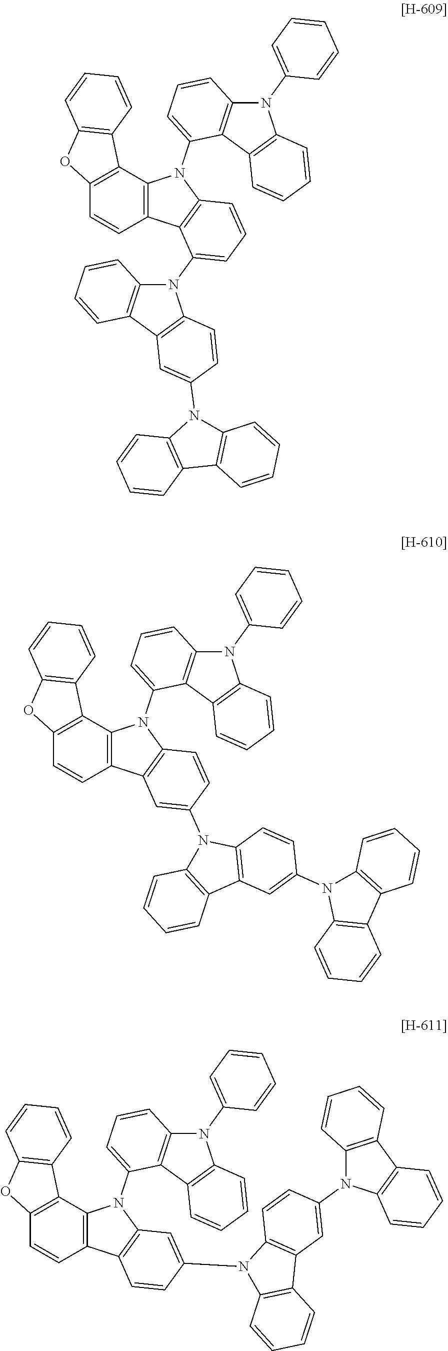 [SMILES: c1ccc(-n2c3ccccc3c3c(-n4c5cccc(-n6c7ccccc7c7cc(-n8c9ccccc9c9ccccc98)ccc76)c5c5ccc6oc7ccccc7c6c54)cccc32)cc1 ; c1ccc(-n2c3ccccc3c3c(-n4c5ccc(-n6c7ccccc7c7cc(-n8c9ccccc9c9ccccc98)ccc76)cc5c5ccc6oc7ccccc7c6c54)cccc32)cc1 ; c1ccc(-n2c3ccccc3c3c(-n4c5cc(-n6c7ccccc7c7cc(-n8c9ccccc9c9ccccc98)ccc76)ccc5c5ccc6oc7ccccc7c6c54)cccc32)cc1]

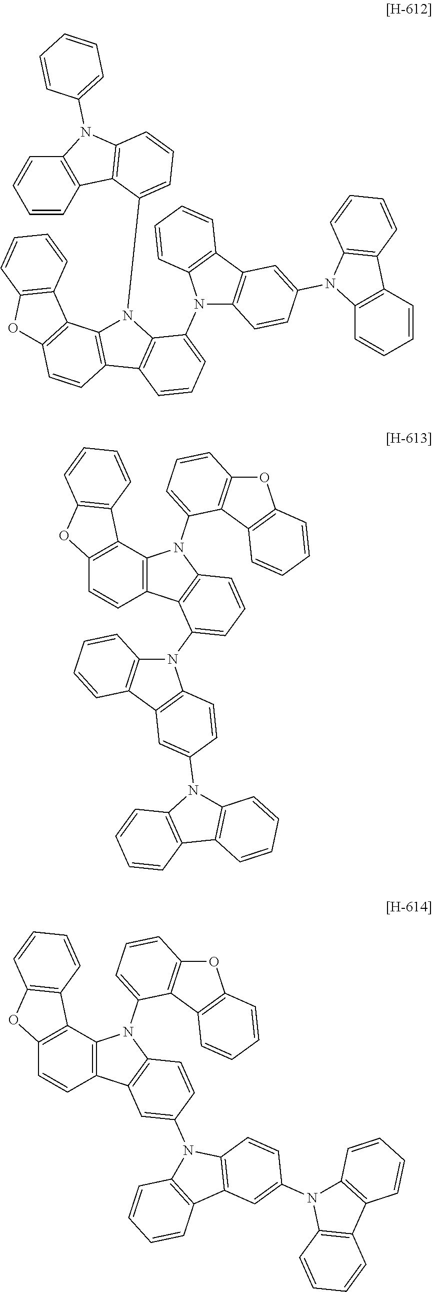 [SMILES: c1ccc(-n2c3ccccc3c3c(-n4c5c(-n6c7ccccc7c7cc(-n8c9ccccc9c9ccccc98)ccc76)cccc5c5ccc6oc7ccccc7c6c54)cccc32)cc1 ; c1ccc2c(c1)oc1cccc(-n3c4cccc(-n5c6ccccc6c6cc(-n7c8ccccc8c8ccccc87)ccc65)c4c4ccc5oc6ccccc6c5c43)c12 ; c1ccc2c(c1)oc1cccc(-n3c4ccc(-n5c6ccccc6c6cc(-n7c8ccccc8c8ccccc87)ccc65)cc4c4ccc5oc6ccccc6c5c43)c12]

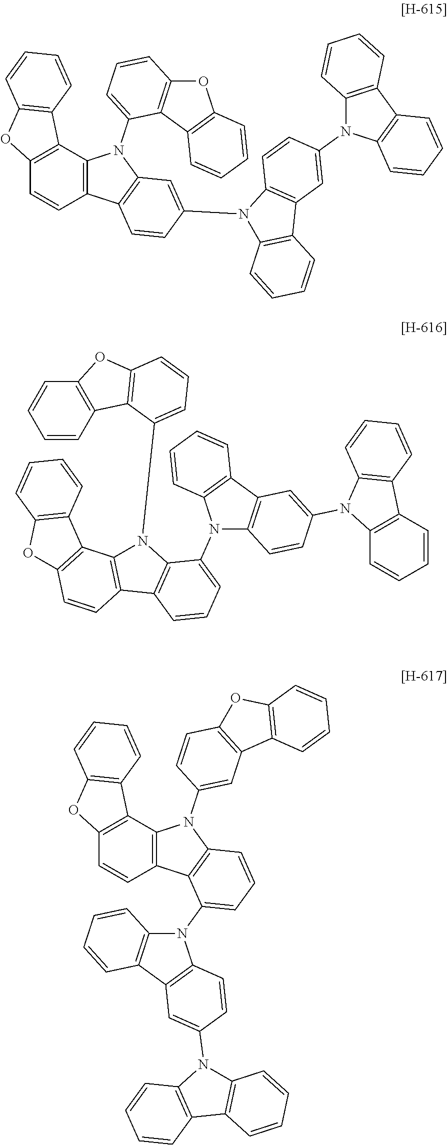 [SMILES: c1ccc2c(c1)oc1cccc(-n3c4cc(-n5c6ccccc6c6cc(-n7c8ccccc8c8ccccc87)ccc65)ccc4c4ccc5oc6ccccc6c5c43)c12 ; c1ccc2c(c1)oc1cccc(-n3c4c(-n5c6ccccc6c6cc(-n7c8ccccc8c8ccccc87)ccc65)cccc4c4ccc5oc6ccccc6c5c43)c12 ; c1ccc2c(c1)oc1ccc(-n3c4cccc(-n5c6ccccc6c6cc(-n7c8ccccc8c8ccccc87)ccc65)c4c4ccc5oc6ccccc6c5c43)cc12]

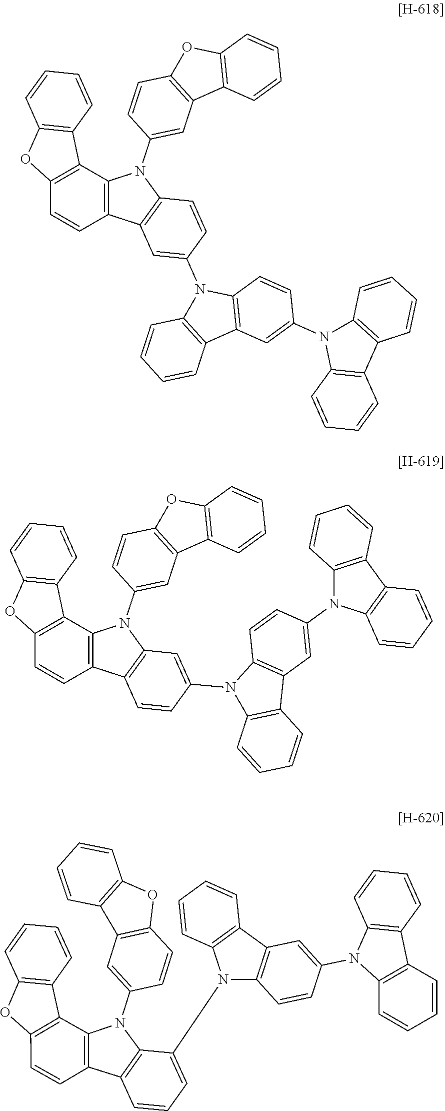 [SMILES: c1ccc2c(c1)oc1ccc(-n3c4ccc(-n5c6ccccc6c6cc(-n7c8ccccc8c8ccccc87)ccc65)cc4c4ccc5oc6ccccc6c5c43)cc12 ; c1ccc2c(c1)oc1ccc(-n3c4cc(-n5c6ccccc6c6cc(-n7c8ccccc8c8ccccc87)ccc65)ccc4c4ccc5oc6ccccc6c5c43)cc12 ; c1ccc2c(c1)oc1ccc(-n3c4c(-n5c6ccccc6c6cc(-n7c8ccccc8c8ccccc87)ccc65)cccc4c4ccc5oc6ccccc6c5c43)cc12]

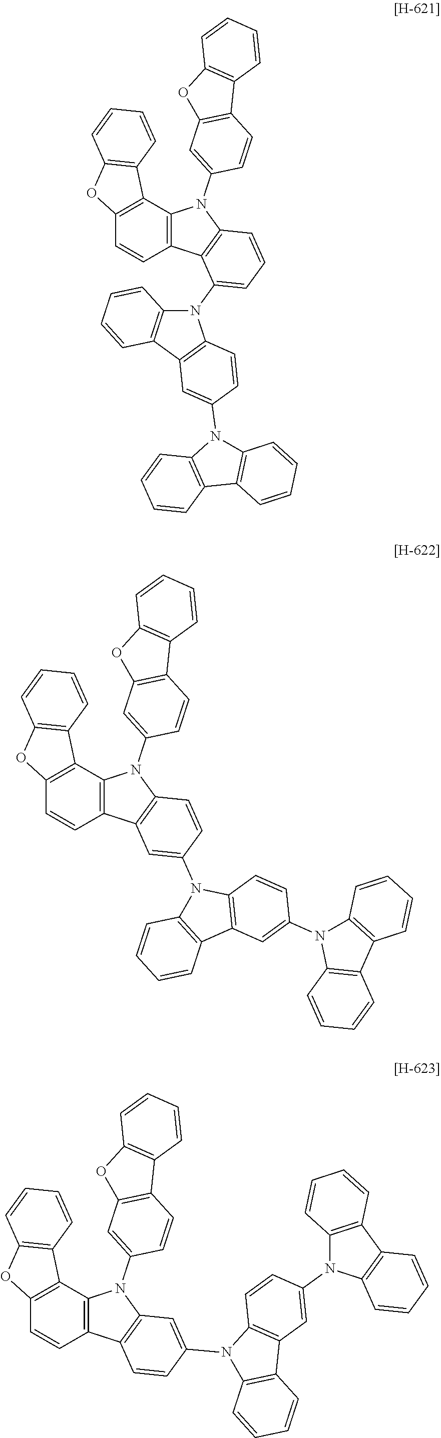 [SMILES: c1ccc2c(c1)oc1cc(-n3c4cccc(-n5c6ccccc6c6cc(-n7c8ccccc8c8ccccc87)ccc65)c4c4ccc5oc6ccccc6c5c43)ccc12 ; c1ccc2c(c1)oc1cc(-n3c4ccc(-n5c6ccccc6c6cc(-n7c8ccccc8c8ccccc87)ccc65)cc4c4ccc5oc6ccccc6c5c43)ccc12 ; c1ccc2c(c1)oc1cc(-n3c4cc(-n5c6ccccc6c6cc(-n7c8ccccc8c8ccccc87)ccc65)ccc4c4ccc5oc6ccccc6c5c43)ccc12]

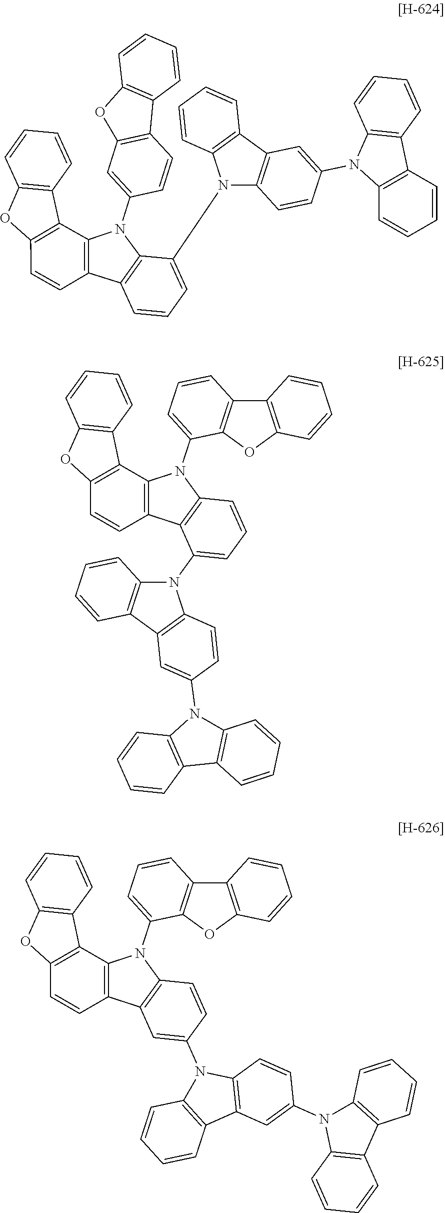 [SMILES: c1ccc2c(c1)oc1cc(-n3c4c(-n5c6ccccc6c6cc(-n7c8ccccc8c8ccccc87)ccc65)cccc4c4ccc5oc6ccccc6c5c43)ccc12 ; c1ccc2c(c1)oc1c(-n3c4cccc(-n5c6ccccc6c6cc(-n7c8ccccc8c8ccccc87)ccc65)c4c4ccc5oc6ccccc6c5c43)cccc12 ; c1ccc2c(c1)oc1c(-n3c4ccc(-n5c6ccccc6c6cc(-n7c8ccccc8c8ccccc87)ccc65)cc4c4ccc5oc6ccccc6c5c43)cccc12]

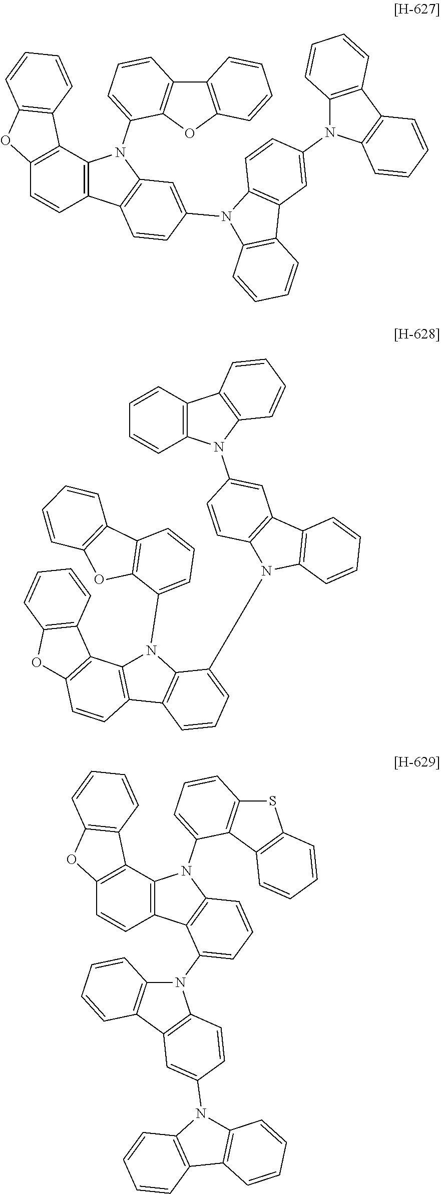 [SMILES: c1ccc2c(c1)oc1c(-n3c4cc(-n5c6ccccc6c6cc(-n7c8ccccc8c8ccccc87)ccc65)ccc4c4ccc5oc6ccccc6c5c43)cccc12 ; c1ccc2c(c1)oc1c(-n3c4c(-n5c6ccccc6c6cc(-n7c8ccccc8c8ccccc87)ccc65)cccc4c4ccc5oc6ccccc6c5c43)cccc12 ; c1ccc2c(c1)oc1ccc3c4c(-n5c6ccccc6c6cc(-n7c8ccccc8c8ccccc87)ccc65)cccc4n(-c4cccc5sc6ccccc6c45)c3c12]

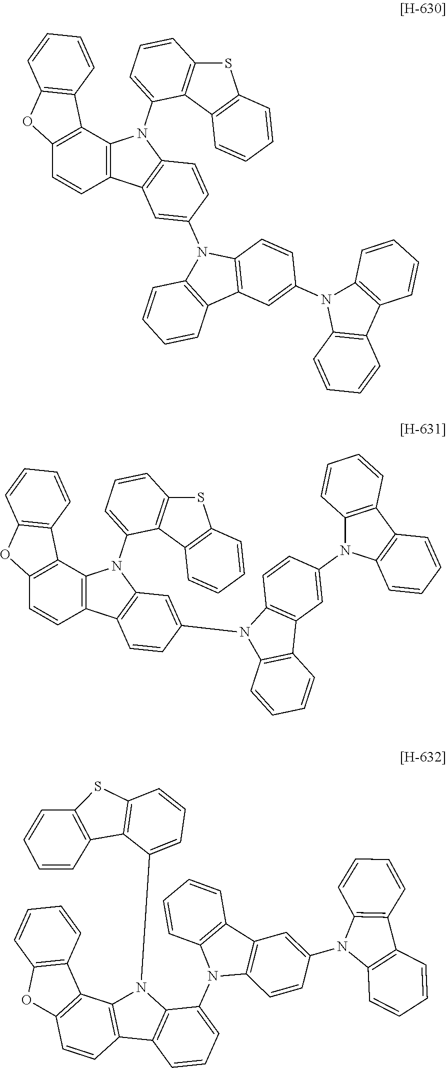 [SMILES: c1ccc2c(c1)oc1ccc3c4cc(-n5c6ccccc6c6cc(-n7c8ccccc8c8ccccc87)ccc65)ccc4n(-c4cccc5sc6ccccc6c45)c3c12 ; c1ccc2c(c1)oc1ccc3c4ccc(-n5c6ccccc6c6cc(-n7c8ccccc8c8ccccc87)ccc65)cc4n(-c4cccc5sc6ccccc6c45)c3c12 ; c1ccc2c(c1)oc1ccc3c4cccc(-n5c6ccccc6c6cc(-n7c8ccccc8c8ccccc87)ccc65)c4n(-c4cccc5sc6ccccc6c45)c3c12]

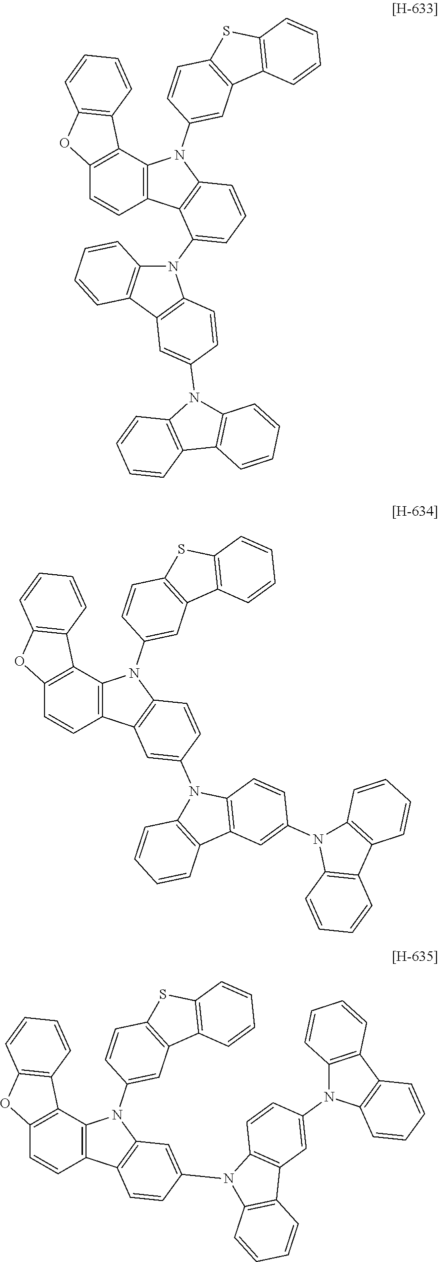 [SMILES: c1ccc2c(c1)oc1ccc3c4c(-n5c6ccccc6c6cc(-n7c8ccccc8c8ccccc87)ccc65)cccc4n(-c4ccc5sc6ccccc6c5c4)c3c12 ; c1ccc2c(c1)oc1ccc3c4cc(-n5c6ccccc6c6cc(-n7c8ccccc8c8ccccc87)ccc65)ccc4n(-c4ccc5sc6ccccc6c5c4)c3c12 ; c1ccc2c(c1)oc1ccc3c4ccc(-n5c6ccccc6c6cc(-n7c8ccccc8c8ccccc87)ccc65)cc4n(-c4ccc5sc6ccccc6c5c4)c3c12]

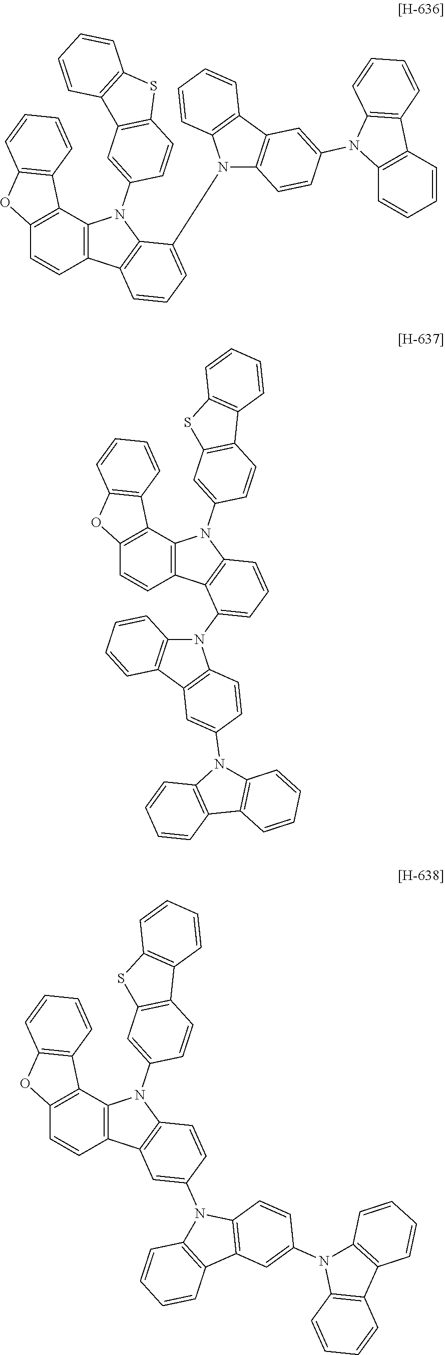 [SMILES: c1ccc2c(c1)oc1ccc3c4cccc(-n5c6ccccc6c6cc(-n7c8ccccc8c8ccccc87)ccc65)c4n(-c4ccc5sc6ccccc6c5c4)c3c12 ; c1ccc2c(c1)oc1ccc3c4c(-n5c6ccccc6c6cc(-n7c8ccccc8c8ccccc87)ccc65)cccc4n(-c4ccc5c(c4)sc4ccccc45)c3c12 ; c1ccc2c(c1)oc1ccc3c4cc(-n5c6ccccc6c6cc(-n7c8ccccc8c8ccccc87)ccc65)ccc4n(-c4ccc5c(c4)sc4ccccc45)c3c12]

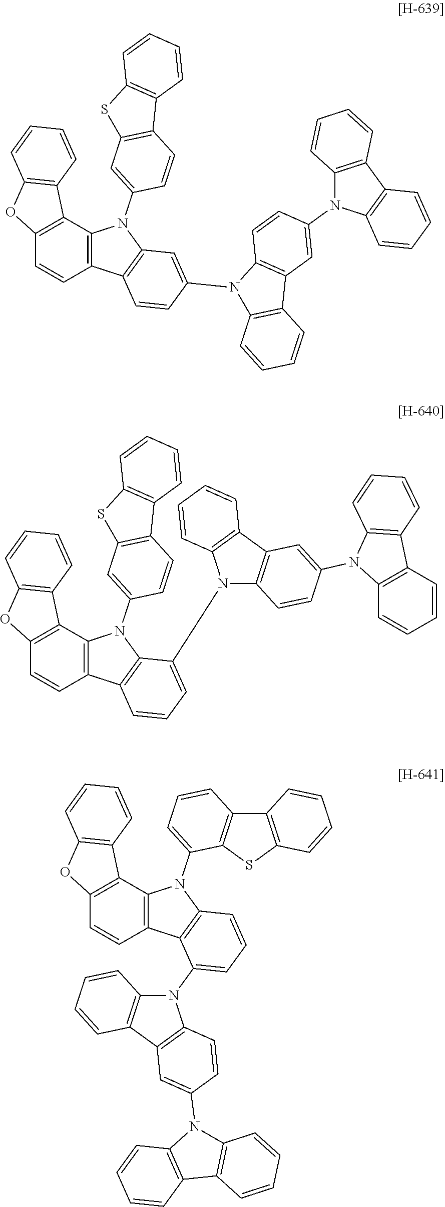 [SMILES: c1ccc2c(c1)oc1ccc3c4ccc(-n5c6ccccc6c6cc(-n7c8ccccc8c8ccccc87)ccc65)cc4n(-c4ccc5c(c4)sc4ccccc45)c3c12 ; c1ccc2c(c1)oc1ccc3c4cccc(-n5c6ccccc6c6cc(-n7c8ccccc8c8ccccc87)ccc65)c4n(-c4ccc5c(c4)sc4ccccc45)c3c12 ; c1ccc2c(c1)oc1ccc3c4c(-n5c6ccccc6c6cc(-n7c8ccccc8c8ccccc87)ccc65)cccc4n(-c4cccc5c4sc4ccccc45)c3c12]

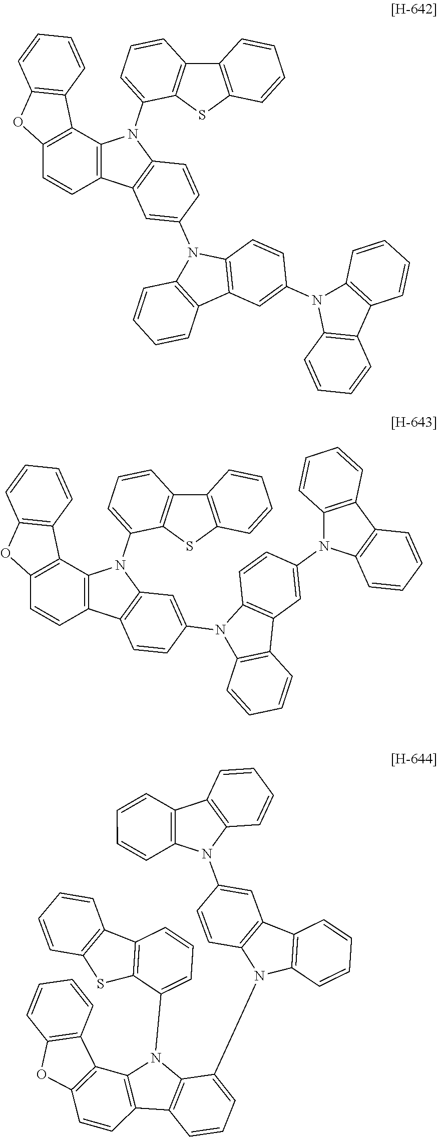 [SMILES: c1ccc2c(c1)oc1ccc3c4cc(-n5c6ccccc6c6cc(-n7c8ccccc8c8ccccc87)ccc65)ccc4n(-c4cccc5c4sc4ccccc45)c3c12 ; c1ccc2c(c1)oc1ccc3c4ccc(-n5c6ccccc6c6cc(-n7c8ccccc8c8ccccc87)ccc65)cc4n(-c4cccc5c4sc4ccccc45)c3c12 ; c1ccc2c(c1)oc1ccc3c4cccc(-n5c6ccccc6c6cc(-n7c8ccccc8c8ccccc87)ccc65)c4n(-c4cccc5c4sc4ccccc45)c3c12]

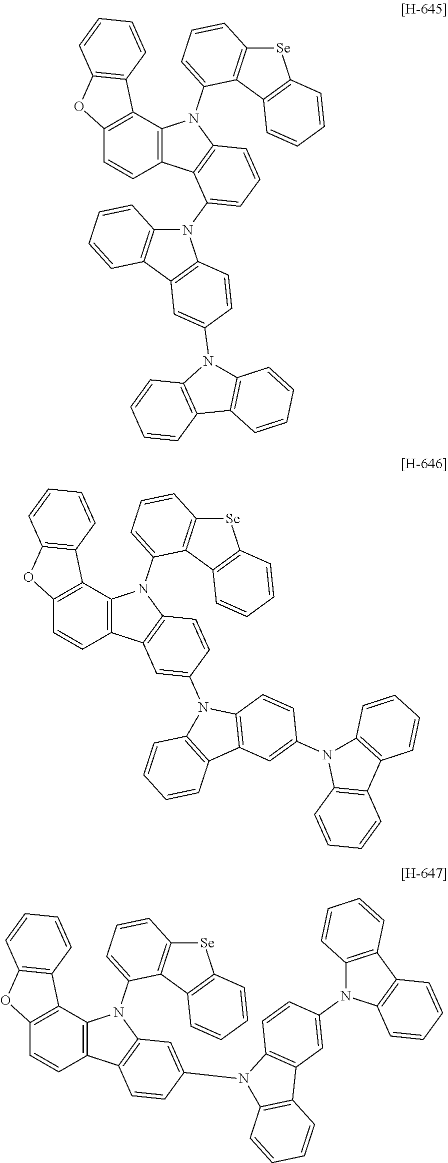 [SMILES: c1ccc2c(c1)oc1ccc3c4c(-n5c6ccccc6c6cc(-n7c8ccccc8c8ccccc87)ccc65)cccc4n(-c4cccc5[se]c6ccccc6c45)c3c12 ; c1ccc2c(c1)oc1ccc3c4cc(-n5c6ccccc6c6cc(-n7c8ccccc8c8ccccc87)ccc65)ccc4n(-c4cccc5[se]c6ccccc6c45)c3c12 ; c1ccc2c(c1)oc1ccc3c4ccc(-n5c6ccccc6c6cc(-n7c8ccccc8c8ccccc87)ccc65)cc4n(-c4cccc5[se]c6ccccc6c45)c3c12]

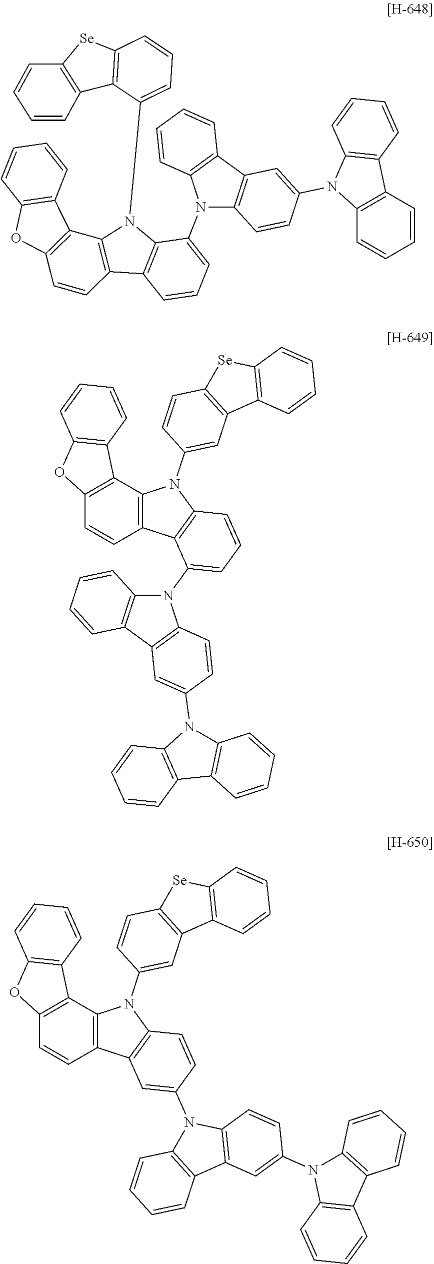 [SMILES: c1ccc2c(c1)oc1ccc3c4cccc(-n5c6ccccc6c6cc(-n7c8ccccc8c8ccccc87)ccc65)c4n(-c4cccc5[se]c6ccccc6c45)c3c12 ; c1ccc2c(c1)oc1ccc3c4c(-n5c6ccccc6c6cc(-n7c8ccccc8c8ccccc87)ccc65)cccc4n(-c4ccc5[se]c6ccccc6c5c4)c3c12 ; c1ccc2c(c1)oc1ccc3c4cc(-n5c6ccccc6c6cc(-n7c8ccccc8c8ccccc87)ccc65)ccc4n(-c4ccc5[se]c6ccccc6c5c4)c3c12]

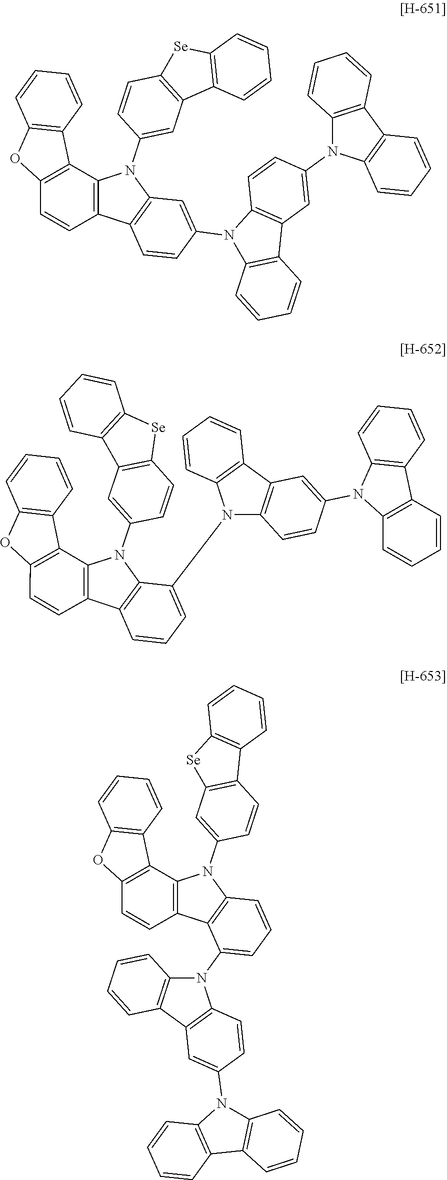 [SMILES: c1ccc2c(c1)oc1ccc3c4ccc(-n5c6ccccc6c6cc(-n7c8ccccc8c8ccccc87)ccc65)cc4n(-c4ccc5[se]c6ccccc6c5c4)c3c12 ; c1ccc2c(c1)oc1ccc3c4cccc(-n5c6ccccc6c6cc(-n7c8ccccc8c8ccccc87)ccc65)c4n(-c4ccc5[se]c6ccccc6c5c4)c3c12 ; c1ccc2c(c1)oc1ccc3c4c(-n5c6ccccc6c6cc(-n7c8ccccc8c8ccccc87)ccc65)cccc4n(-c4ccc5c(c4)[se]c4ccccc45)c3c12]

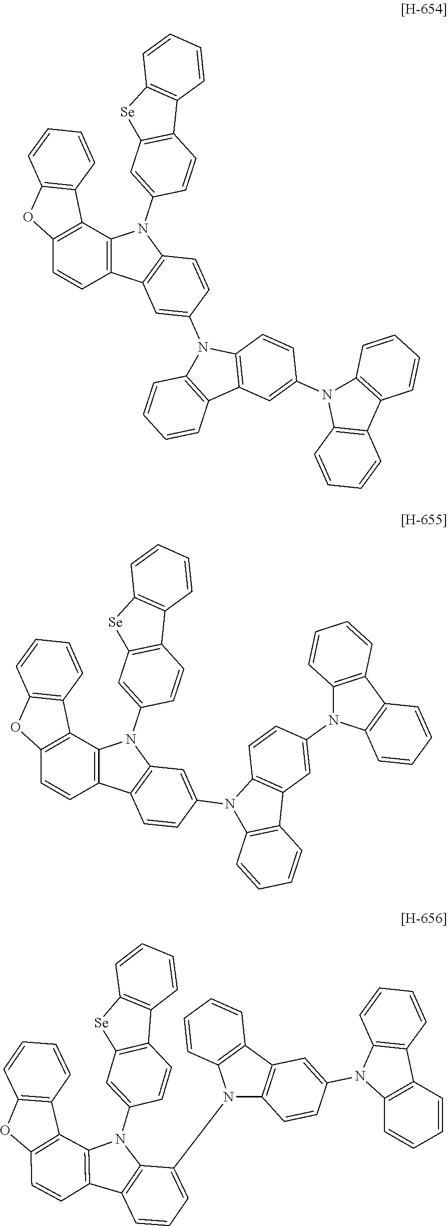 [SMILES: c1ccc2c(c1)oc1ccc3c4cc(-n5c6ccccc6c6cc(-n7c8ccccc8c8ccccc87)ccc65)ccc4n(-c4ccc5c(c4)[se]c4ccccc45)c3c12 ; c1ccc2c(c1)oc1ccc3c4ccc(-n5c6ccccc6c6cc(-n7c8ccccc8c8ccccc87)ccc65)cc4n(-c4ccc5c(c4)[se]c4ccccc45)c3c12 ; c1ccc2c(c1)oc1ccc3c4cccc(-n5c6ccccc6c6cc(-n7c8ccccc8c8ccccc87)ccc65)c4n(-c4ccc5c(c4)[se]c4ccccc45)c3c12]

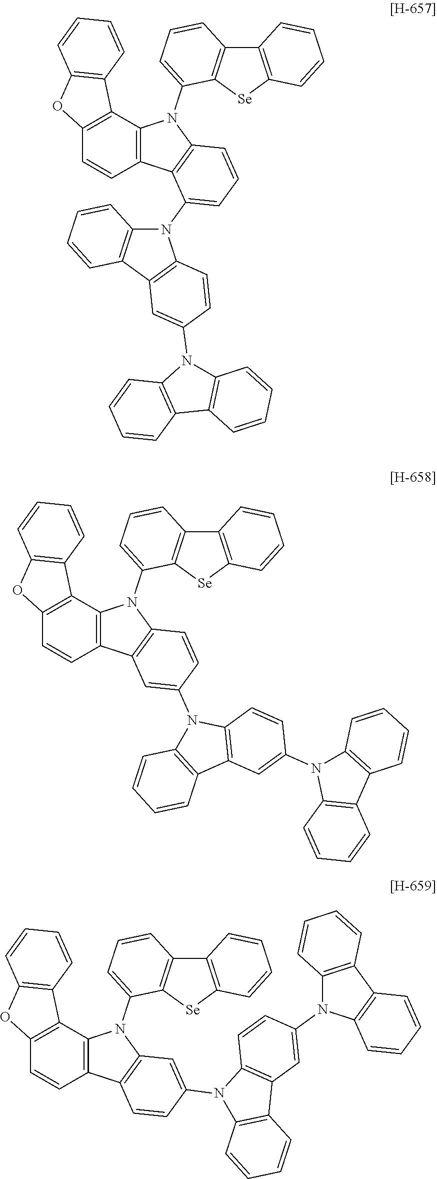 [SMILES: c1ccc2c(c1)oc1ccc3c4c(-n5c6ccccc6c6cc(-n7c8ccccc8c8ccccc87)ccc65)cccc4n(-c4cccc5c4[se]c4ccccc45)c3c12 ; c1ccc2c(c1)oc1ccc3c4cc(-n5c6ccccc6c6cc(-n7c8ccccc8c8ccccc87)ccc65)ccc4n(-c4cccc5c4[se]c4ccccc45)c3c12 ; c1ccc2c(c1)oc1ccc3c4ccc(-n5c6ccccc6c6cc(-n7c8ccccc8c8ccccc87)ccc65)cc4n(-c4cccc5c4[se]c4ccccc45)c3c12]

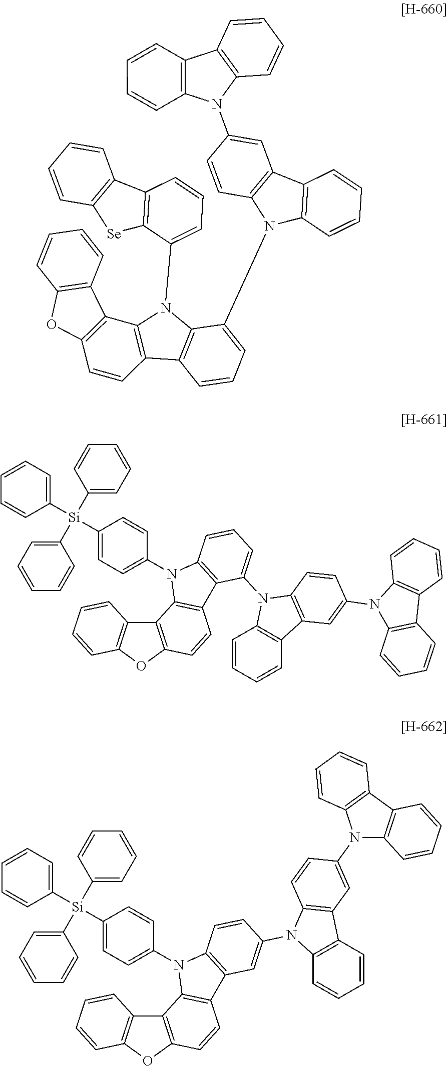 [SMILES: c1ccc2c(c1)oc1ccc3c4cccc(-n5c6ccccc6c6cc(-n7c8ccccc8c8ccccc87)ccc65)c4n(-c4cccc5c4[se]c4ccccc45)c3c12 ; c1ccc([Si](c2ccccc2)(c2ccccc2)c2ccc(-n3c4cccc(-n5c6ccccc6c6cc(-n7c8ccccc8c8ccccc87)ccc65)c4c4ccc5oc6ccccc6c5c43)cc2)cc1 ; c1ccc([Si](c2ccccc2)(c2ccccc2)c2ccc(-n3c4ccc(-n5c6ccccc6c6cc(-n7c8ccccc8c8ccccc87)ccc65)cc4c4ccc5oc6ccccc6c5c43)cc2)cc1]

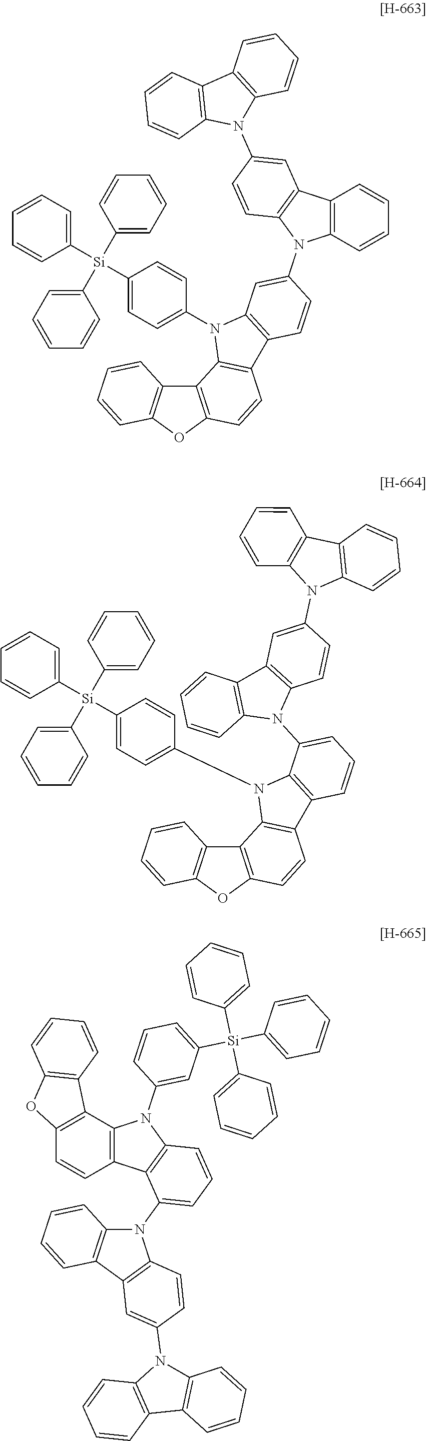

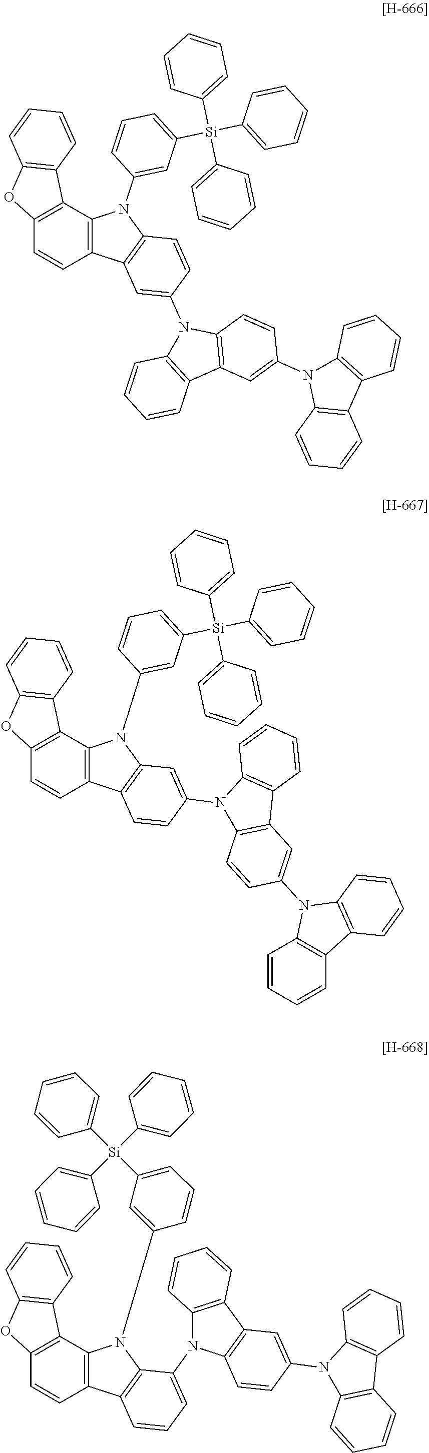 [SMILES: c1ccc([Si](c2ccccc2)(c2ccccc2)c2cccc(-n3c4ccc(-n5c6ccccc6c6cc(-n7c8ccccc8c8ccccc87)ccc65)cc4c4ccc5oc6ccccc6c5c43)c2)cc1 ; c1ccc([Si](c2ccccc2)(c2ccccc2)c2cccc(-n3c4cc(-n5c6ccccc6c6cc(-n7c8ccccc8c8ccccc87)ccc65)ccc4c4ccc5oc6ccccc6c5c43)c2)cc1 ; c1ccc([Si](c2ccccc2)(c2ccccc2)c2cccc(-n3c4c(-n5c6ccccc6c6cc(-n7c8ccccc8c8ccccc87)ccc65)cccc4c4ccc5oc6ccccc6c5c43)c2)cc1]

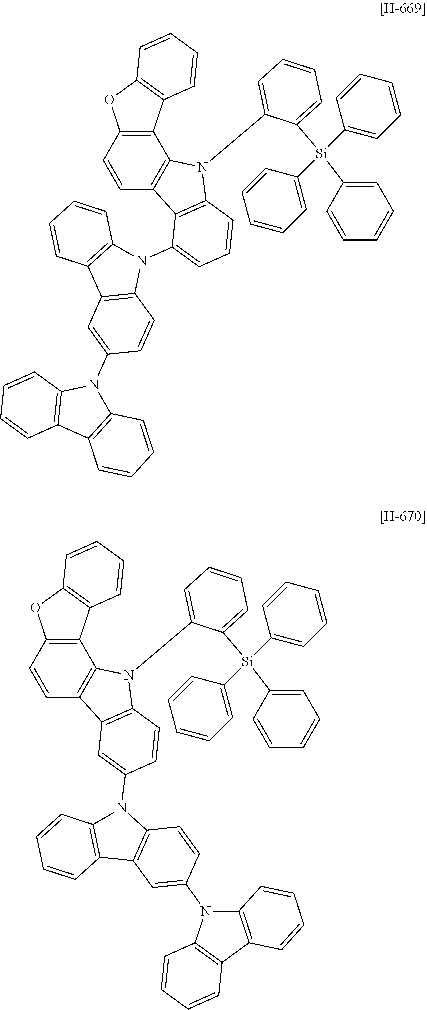 [SMILES: c1ccc([Si](c2ccccc2)(c2ccccc2)c2ccccc2-n2c3cccc(-n4c5ccccc5c5cc(-n6c7ccccc7c7ccccc76)ccc54)c3c3ccc4oc5ccccc5c4c32)cc1 ; c1ccc([Si](c2ccccc2)(c2ccccc2)c2ccccc2-n2c3ccc(-n4c5ccccc5c5cc(-n6c7ccccc7c7ccccc76)ccc54)cc3c3ccc4oc5ccccc5c4c32)cc1]

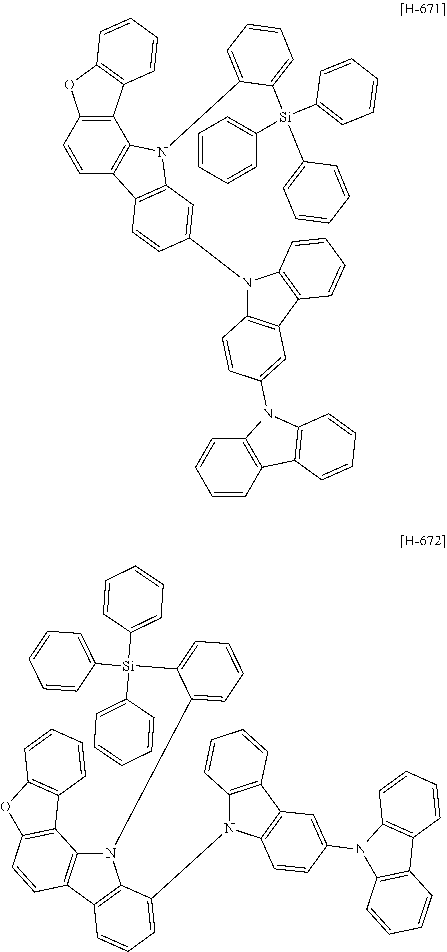 [SMILES: c1ccc([Si](c2ccccc2)(c2ccccc2)c2ccccc2-n2c3cc(-n4c5ccccc5c5cc(-n6c7ccccc7c7ccccc76)ccc54)ccc3c3ccc4oc5ccccc5c4c32)cc1 ; c1ccc([Si](c2ccccc2)(c2ccccc2)c2ccccc2-n2c3c(-n4c5ccccc5c5cc(-n6c7ccccc7c7ccccc76)ccc54)cccc3c3ccc4oc5ccccc5c4c32)cc1]

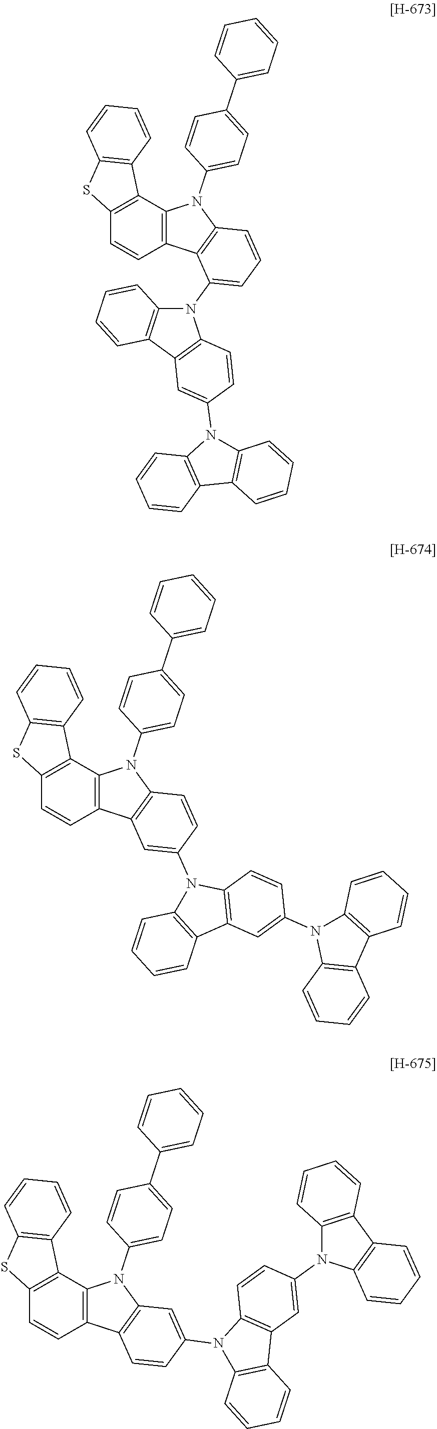 [SMILES: c1ccc(-c2ccc(-n3c4cccc(-n5c6ccccc6c6cc(-n7c8ccccc8c8ccccc87)ccc65)c4c4ccc5sc6ccccc6c5c43)cc2)cc1 ; c1ccc(-c2ccc(-n3c4ccc(-n5c6ccccc6c6cc(-n7c8ccccc8c8ccccc87)ccc65)cc4c4ccc5sc6ccccc6c5c43)cc2)cc1 ; c1ccc(-c2ccc(-n3c4cc(-n5c6ccccc6c6cc(-n7c8ccccc8c8ccccc87)ccc65)ccc4c4ccc5sc6ccccc6c5c43)cc2)cc1]

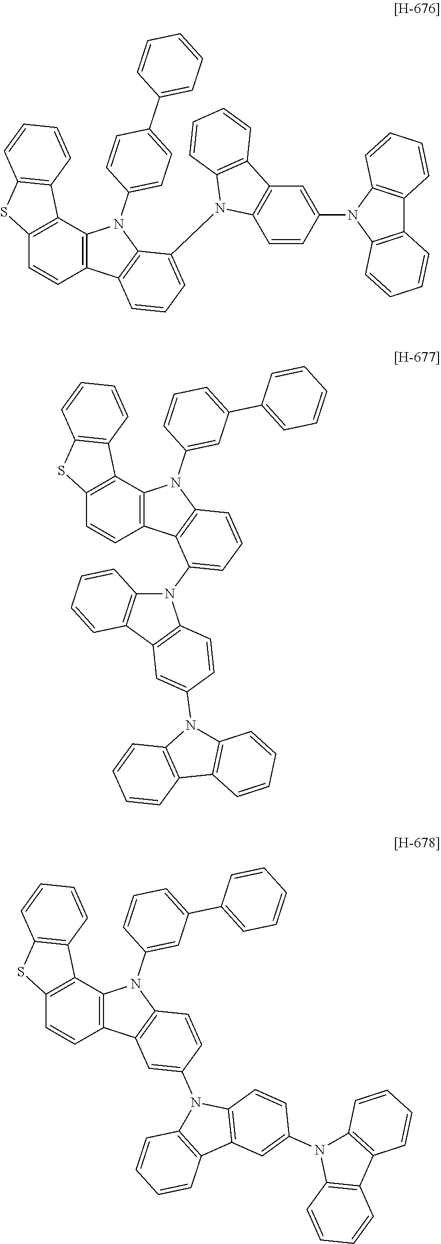 [SMILES: c1ccc(-c2ccc(-n3c4c(-n5c6ccccc6c6cc(-n7c8ccccc8c8ccccc87)ccc65)cccc4c4ccc5sc6ccccc6c5c43)cc2)cc1 ; c1ccc(-c2cccc(-n3c4cccc(-n5c6ccccc6c6cc(-n7c8ccccc8c8ccccc87)ccc65)c4c4ccc5sc6ccccc6c5c43)c2)cc1 ; c1ccc(-c2cccc(-n3c4ccc(-n5c6ccccc6c6cc(-n7c8ccccc8c8ccccc87)ccc65)cc4c4ccc5sc6ccccc6c5c43)c2)cc1]

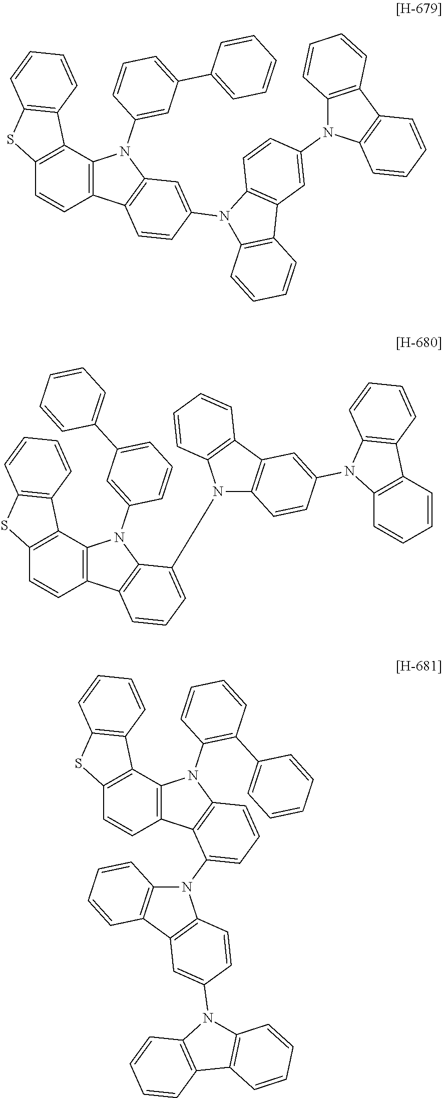 [SMILES: c1ccc(-c2cccc(-n3c4cc(-n5c6ccccc6c6cc(-n7c8ccccc8c8ccccc87)ccc65)ccc4c4ccc5sc6ccccc6c5c43)c2)cc1 ; c1ccc(-c2cccc(-n3c4c(-n5c6ccccc6c6cc(-n7c8ccccc8c8ccccc87)ccc65)cccc4c4ccc5sc6ccccc6c5c43)c2)cc1 ; c1ccc(-c2ccccc2-n2c3cccc(-n4c5ccccc5c5cc(-n6c7ccccc7c7ccccc76)ccc54)c3c3ccc4sc5ccccc5c4c32)cc1]

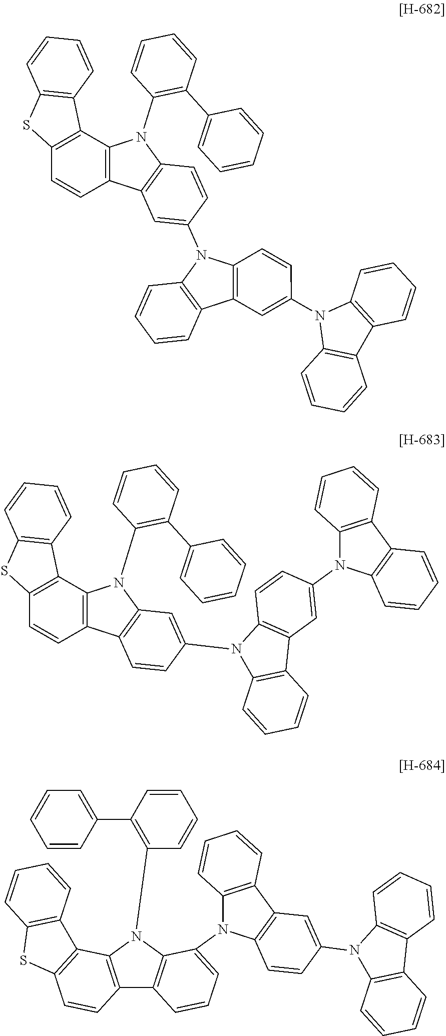 [SMILES: c1ccc(-c2ccccc2-n2c3ccc(-n4c5ccccc5c5cc(-n6c7ccccc7c7ccccc76)ccc54)cc3c3ccc4sc5ccccc5c4c32)cc1 ; c1ccc(-c2ccccc2-n2c3cc(-n4c5ccccc5c5cc(-n6c7ccccc7c7ccccc76)ccc54)ccc3c3ccc4sc5ccccc5c4c32)cc1 ; c1ccc(-c2ccccc2-n2c3c(-n4c5ccccc5c5cc(-n6c7ccccc7c7ccccc76)ccc54)cccc3c3ccc4sc5ccccc5c4c32)cc1]

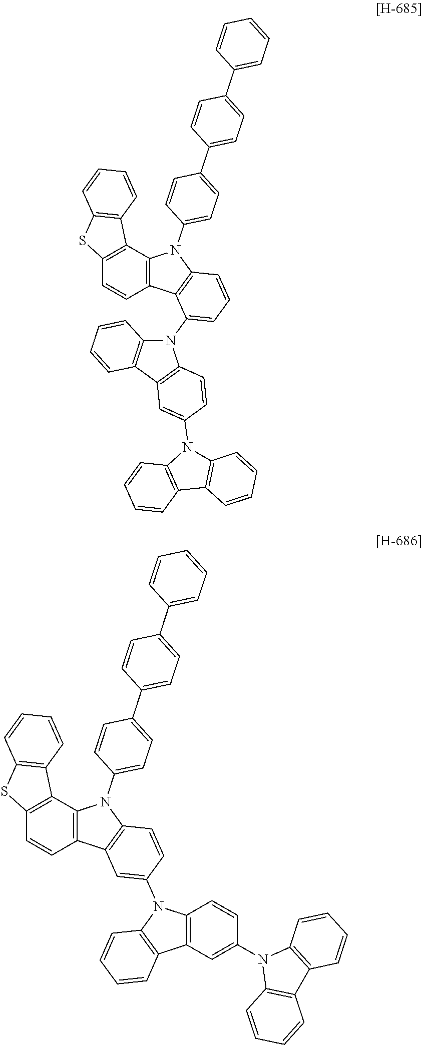 [SMILES: c1ccc(-c2ccc(-c3ccc(-n4c5cccc(-n6c7ccccc7c7cc(-n8c9ccccc9c9ccccc98)ccc76)c5c5ccc6sc7ccccc7c6c54)cc3)cc2)cc1 ; c1ccc(-c2ccc(-c3ccc(-n4c5ccc(-n6c7ccccc7c7cc(-n8c9ccccc9c9ccccc98)ccc76)cc5c5ccc6sc7ccccc7c6c54)cc3)cc2)cc1]

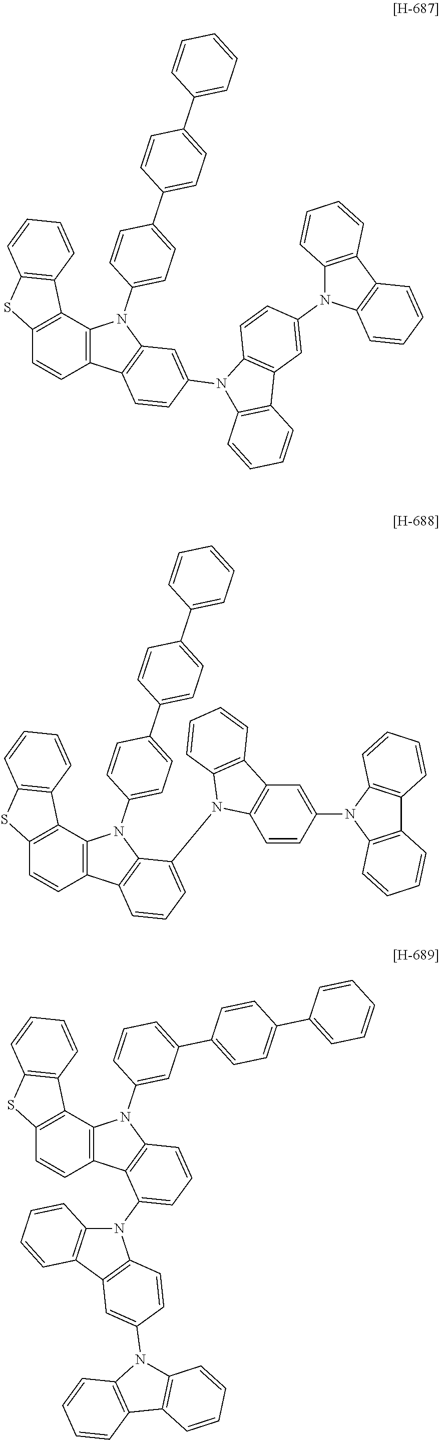 [SMILES: c1ccc(-c2ccc(-c3ccc(-n4c5cc(-n6c7ccccc7c7cc(-n8c9ccccc9c9ccccc98)ccc76)ccc5c5ccc6sc7ccccc7c6c54)cc3)cc2)cc1 ; c1ccc(-c2ccc(-c3ccc(-n4c5c(-n6c7ccccc7c7cc(-n8c9ccccc9c9ccccc98)ccc76)cccc5c5ccc6sc7ccccc7c6c54)cc3)cc2)cc1 ; c1ccc(-c2ccc(-c3cccc(-n4c5cccc(-n6c7ccccc7c7cc(-n8c9ccccc9c9ccccc98)ccc76)c5c5ccc6sc7ccccc7c6c54)c3)cc2)cc1]

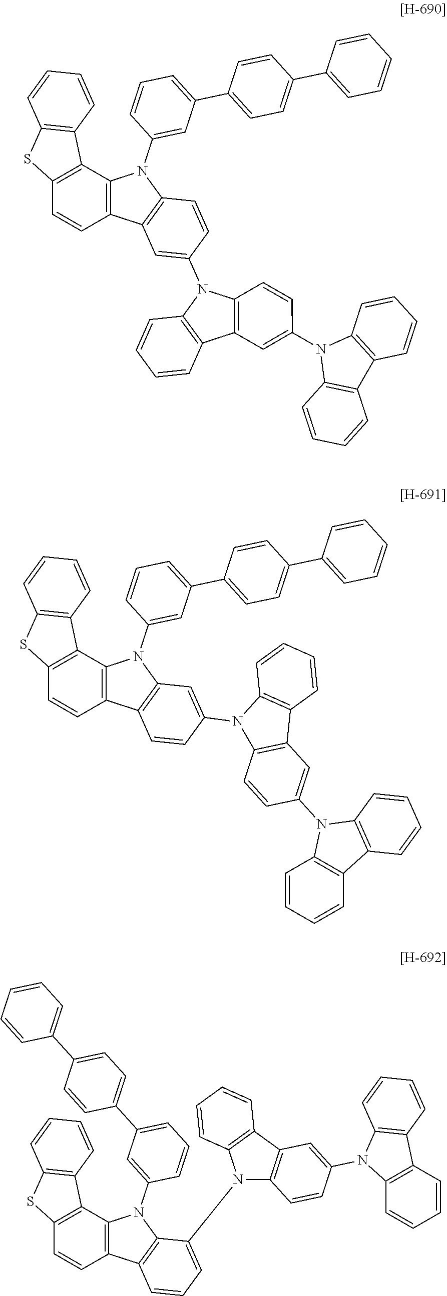 [SMILES: c1ccc(-c2ccc(-c3cccc(-n4c5ccc(-n6c7ccccc7c7cc(-n8c9ccccc9c9ccccc98)ccc76)cc5c5ccc6sc7ccccc7c6c54)c3)cc2)cc1 ; c1ccc(-c2ccc(-c3cccc(-n4c5cc(-n6c7ccccc7c7cc(-n8c9ccccc9c9ccccc98)ccc76)ccc5c5ccc6sc7ccccc7c6c54)c3)cc2)cc1 ; c1ccc(-c2ccc(-c3cccc(-n4c5c(-n6c7ccccc7c7cc(-n8c9ccccc9c9ccccc98)ccc76)cccc5c5ccc6sc7ccccc7c6c54)c3)cc2)cc1]

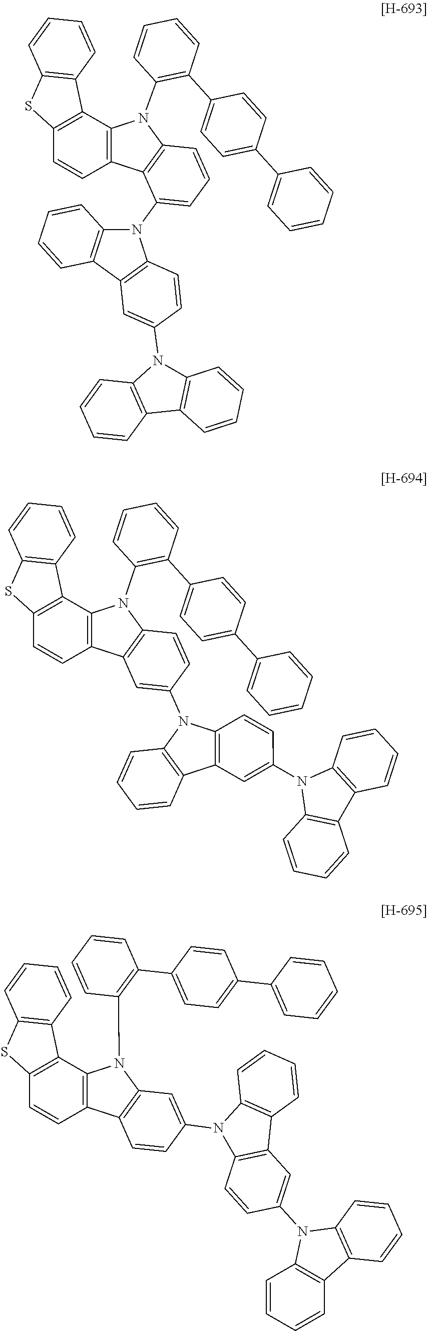 [SMILES: c1ccc(-c2ccc(-c3ccccc3-n3c4cccc(-n5c6ccccc6c6cc(-n7c8ccccc8c8ccccc87)ccc65)c4c4ccc5sc6ccccc6c5c43)cc2)cc1 ; c1ccc(-c2ccc(-c3ccccc3-n3c4ccc(-n5c6ccccc6c6cc(-n7c8ccccc8c8ccccc87)ccc65)cc4c4ccc5sc6ccccc6c5c43)cc2)cc1 ; c1ccc(-c2ccc(-c3ccccc3-n3c4cc(-n5c6ccccc6c6cc(-n7c8ccccc8c8ccccc87)ccc65)ccc4c4ccc5sc6ccccc6c5c43)cc2)cc1]

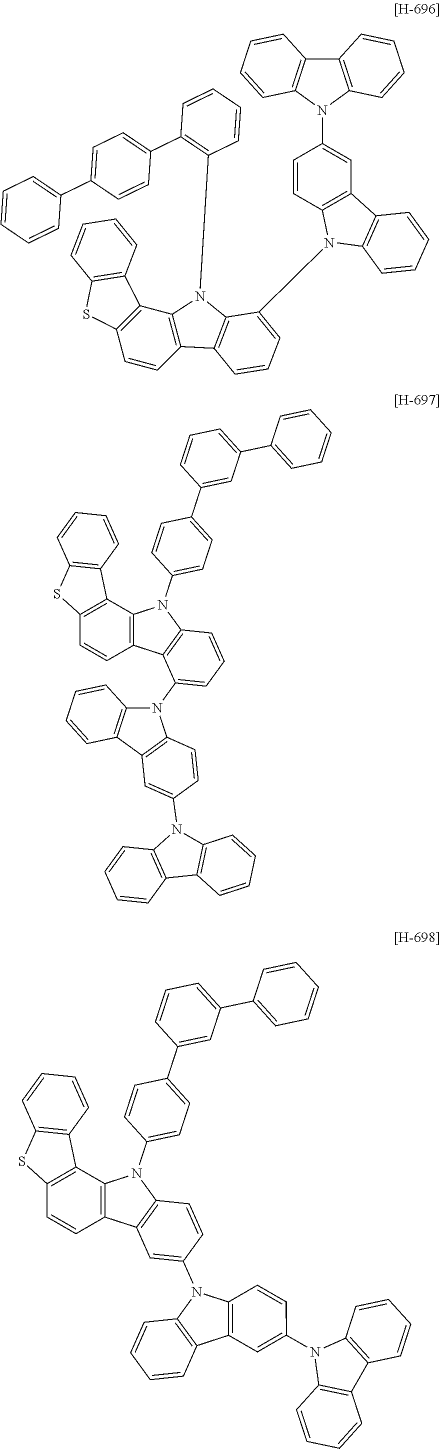 [SMILES: c1ccc(-c2ccc(-c3ccccc3-n3c4c(-n5c6ccccc6c6cc(-n7c8ccccc8c8ccccc87)ccc65)cccc4c4ccc5sc6ccccc6c5c43)cc2)cc1 ; c1ccc(-c2cccc(-c3ccc(-n4c5cccc(-n6c7ccccc7c7cc(-n8c9ccccc9c9ccccc98)ccc76)c5c5ccc6sc7ccccc7c6c54)cc3)c2)cc1 ; c1ccc(-c2cccc(-c3ccc(-n4c5ccc(-n6c7ccccc7c7cc(-n8c9ccccc9c9ccccc98)ccc76)cc5c5ccc6sc7ccccc7c6c54)cc3)c2)cc1]

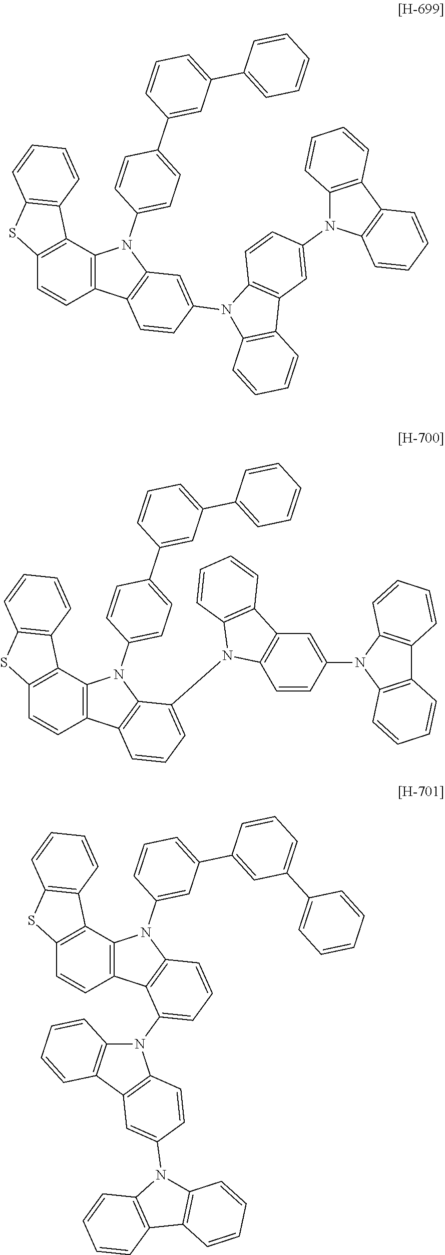 [SMILES: c1ccc(-c2cccc(-c3ccc(-n4c5cc(-n6c7ccccc7c7cc(-n8c9ccccc9c9ccccc98)ccc76)ccc5c5ccc6sc7ccccc7c6c54)cc3)c2)cc1 ; c1ccc(-c2cccc(-c3ccc(-n4c5c(-n6c7ccccc7c7cc(-n8c9ccccc9c9ccccc98)ccc76)cccc5c5ccc6sc7ccccc7c6c54)cc3)c2)cc1 ; c1ccc(-c2cccc(-c3cccc(-n4c5cccc(-n6c7ccccc7c7cc(-n8c9ccccc9c9ccccc98)ccc76)c5c5ccc6sc7ccccc7c6c54)c3)c2)cc1]

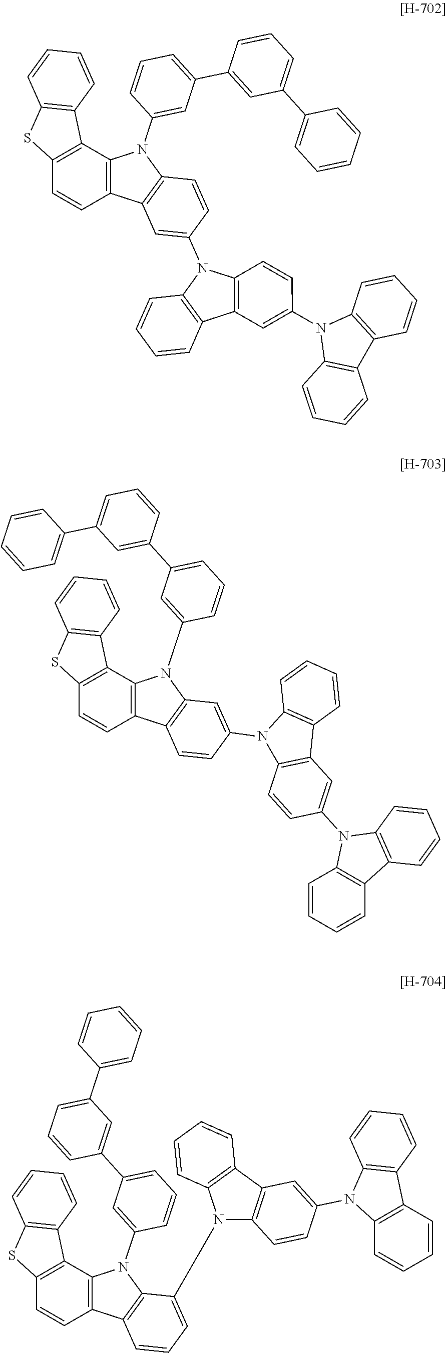 [SMILES: c1ccc(-c2cccc(-c3cccc(-n4c5ccc(-n6c7ccccc7c7cc(-n8c9ccccc9c9ccccc98)ccc76)cc5c5ccc6sc7ccccc7c6c54)c3)c2)cc1 ; c1ccc(-c2cccc(-c3cccc(-n4c5cc(-n6c7ccccc7c7cc(-n8c9ccccc9c9ccccc98)ccc76)ccc5c5ccc6sc7ccccc7c6c54)c3)c2)cc1 ; c1ccc(-c2cccc(-c3cccc(-n4c5c(-n6c7ccccc7c7cc(-n8c9ccccc9c9ccccc98)ccc76)cccc5c5ccc6sc7ccccc7c6c54)c3)c2)cc1]

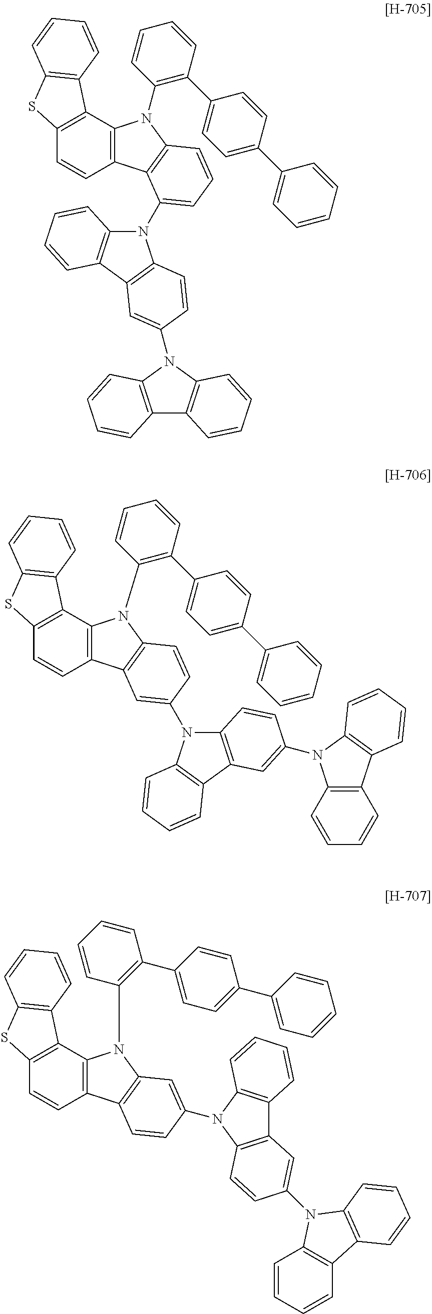 [SMILES: c1ccc(-c2ccc(-c3ccccc3-n3c4cccc(-n5c6ccccc6c6cc(-n7c8ccccc8c8ccccc87)ccc65)c4c4ccc5sc6ccccc6c5c43)cc2)cc1 ; c1ccc(-c2ccc(-c3ccccc3-n3c4ccc(-n5c6ccccc6c6cc(-n7c8ccccc8c8ccccc87)ccc65)cc4c4ccc5sc6ccccc6c5c43)cc2)cc1 ; c1ccc(-c2ccc(-c3ccccc3-n3c4cc(-n5c6ccccc6c6cc(-n7c8ccccc8c8ccccc87)ccc65)ccc4c4ccc5sc6ccccc6c5c43)cc2)cc1]

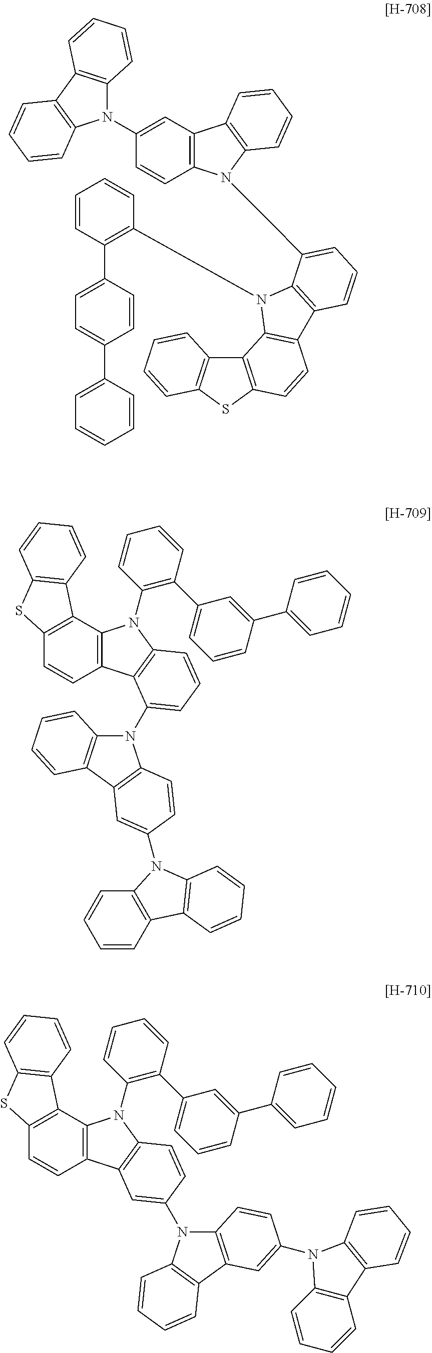 [SMILES: c1ccc(-c2ccc(-c3ccccc3-n3c4c(-n5c6ccccc6c6cc(-n7c8ccccc8c8ccccc87)ccc65)cccc4c4ccc5sc6ccccc6c5c43)cc2)cc1 ; c1ccc(-c2cccc(-c3ccccc3-n3c4cccc(-n5c6ccccc6c6cc(-n7c8ccccc8c8ccccc87)ccc65)c4c4ccc5sc6ccccc6c5c43)c2)cc1 ; c1ccc(-c2cccc(-c3ccccc3-n3c4ccc(-n5c6ccccc6c6cc(-n7c8ccccc8c8ccccc87)ccc65)cc4c4ccc5sc6ccccc6c5c43)c2)cc1]

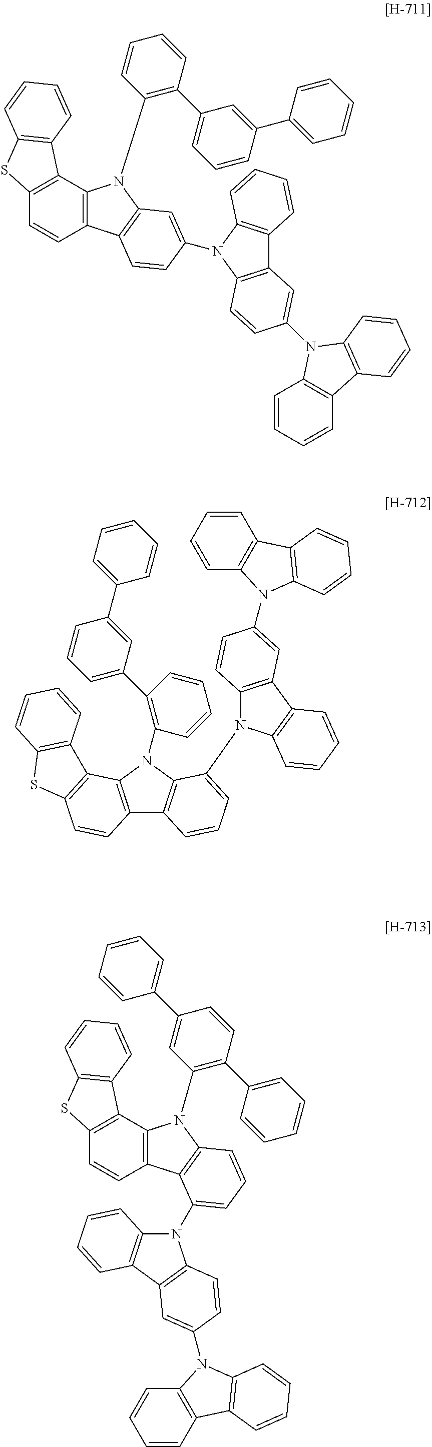 [SMILES: c1ccc(-c2cccc(-c3ccccc3-n3c4cc(-n5c6ccccc6c6cc(-n7c8ccccc8c8ccccc87)ccc65)ccc4c4ccc5sc6ccccc6c5c43)c2)cc1 ; c1ccc(-c2cccc(-c3ccccc3-n3c4c(-n5c6ccccc6c6cc(-n7c8ccccc8c8ccccc87)ccc65)cccc4c4ccc5sc6ccccc6c5c43)c2)cc1 ; c1ccc(-c2ccc(-c3ccccc3)c(-n3c4cccc(-n5c6ccccc6c6cc(-n7c8ccccc8c8ccccc87)ccc65)c4c4ccc5sc6ccccc6c5c43)c2)cc1]

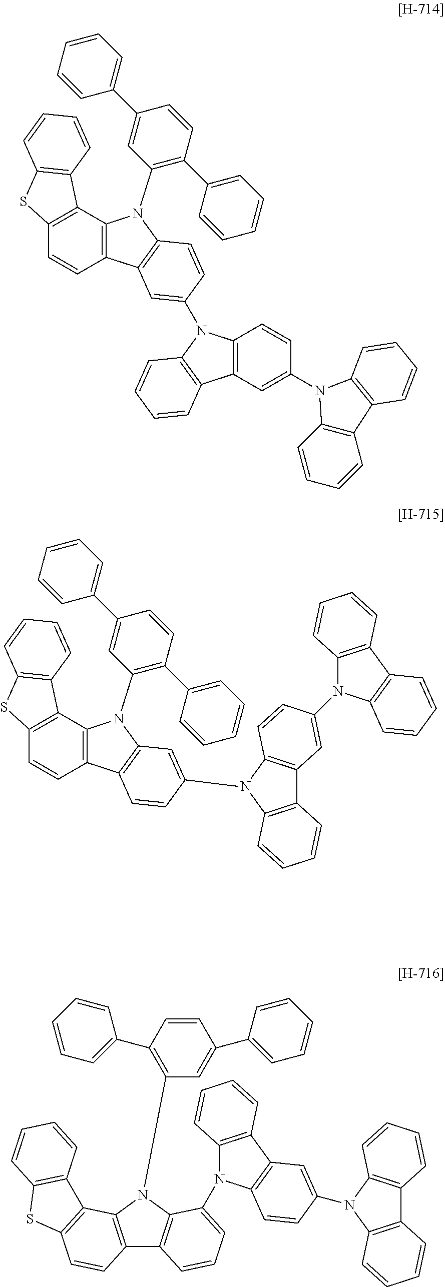 [SMILES: c1ccc(-c2ccc(-c3ccccc3)c(-n3c4ccc(-n5c6ccccc6c6cc(-n7c8ccccc8c8ccccc87)ccc65)cc4c4ccc5sc6ccccc6c5c43)c2)cc1 ; c1ccc(-c2ccc(-c3ccccc3)c(-n3c4cc(-n5c6ccccc6c6cc(-n7c8ccccc8c8ccccc87)ccc65)ccc4c4ccc5sc6ccccc6c5c43)c2)cc1 ; c1ccc(-c2ccc(-c3ccccc3)c(-n3c4c(-n5c6ccccc6c6cc(-n7c8ccccc8c8ccccc87)ccc65)cccc4c4ccc5sc6ccccc6c5c43)c2)cc1]

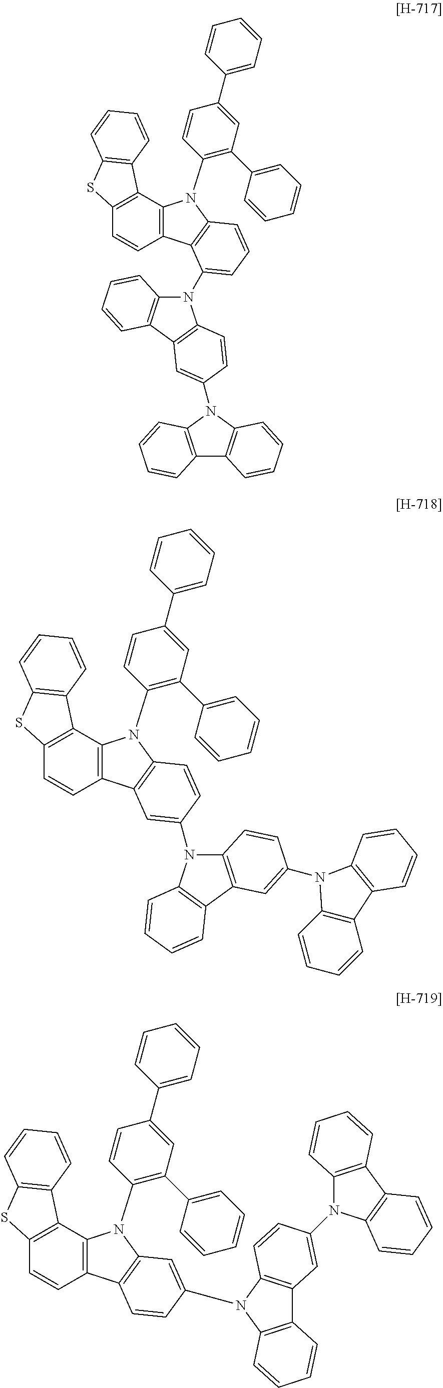 [SMILES: c1ccc(-c2ccc(-n3c4cccc(-n5c6ccccc6c6cc(-n7c8ccccc8c8ccccc87)ccc65)c4c4ccc5sc6ccccc6c5c43)c(-c3ccccc3)c2)cc1 ; c1ccc(-c2ccc(-n3c4ccc(-n5c6ccccc6c6cc(-n7c8ccccc8c8ccccc87)ccc65)cc4c4ccc5sc6ccccc6c5c43)c(-c3ccccc3)c2)cc1 ; c1ccc(-c2ccc(-n3c4cc(-n5c6ccccc6c6cc(-n7c8ccccc8c8ccccc87)ccc65)ccc4c4ccc5sc6ccccc6c5c43)c(-c3ccccc3)c2)cc1]

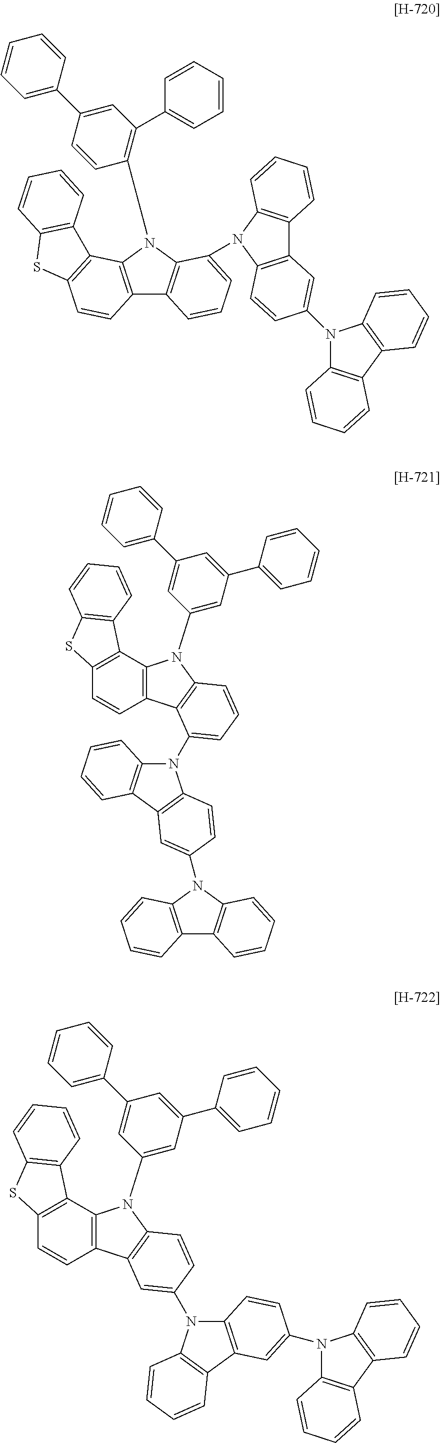 [SMILES: c1ccc(-c2ccc(-n3c4c(-n5c6ccccc6c6cc(-n7c8ccccc8c8ccccc87)ccc65)cccc4c4ccc5sc6ccccc6c5c43)c(-c3ccccc3)c2)cc1 ; c1ccc(-c2cc(-c3ccccc3)cc(-n3c4cccc(-n5c6ccccc6c6cc(-n7c8ccccc8c8ccccc87)ccc65)c4c4ccc5sc6ccccc6c5c43)c2)cc1 ; c1ccc(-c2cc(-c3ccccc3)cc(-n3c4ccc(-n5c6ccccc6c6cc(-n7c8ccccc8c8ccccc87)ccc65)cc4c4ccc5sc6ccccc6c5c43)c2)cc1]

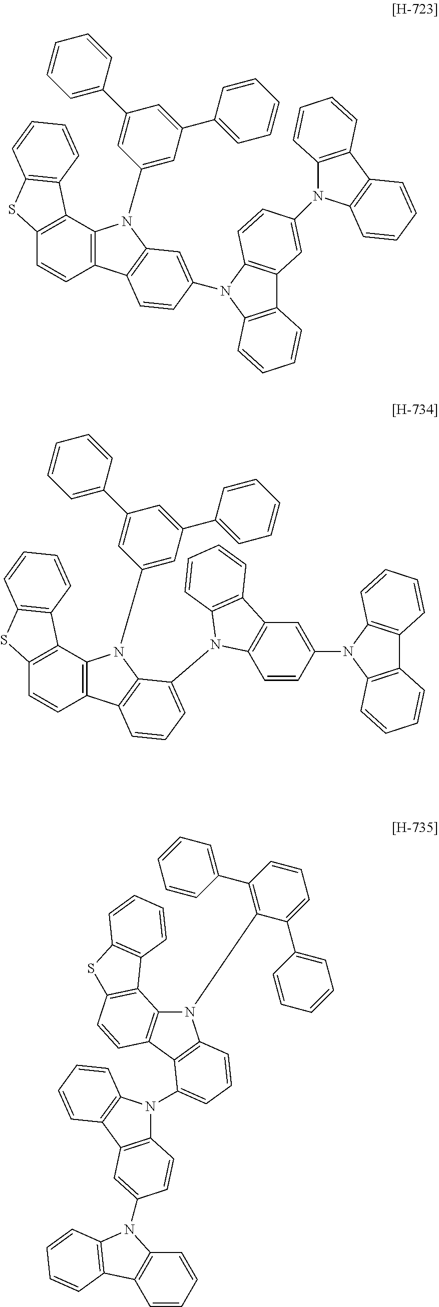 [SMILES: c1ccc(-c2cc(-c3ccccc3)cc(-n3c4cc(-n5c6ccccc6c6cc(-n7c8ccccc8c8ccccc87)ccc65)ccc4c4ccc5sc6ccccc6c5c43)c2)cc1 ; c1ccc(-c2cc(-c3ccccc3)cc(-n3c4c(-n5c6ccccc6c6cc(-n7c8ccccc8c8ccccc87)ccc65)cccc4c4ccc5sc6ccccc6c5c43)c2)cc1 ; c1ccc(-c2cccc(-c3ccccc3)c2-n2c3cccc(-n4c5ccccc5c5cc(-n6c7ccccc7c7ccccc76)ccc54)c3c3ccc4sc5ccccc5c4c32)cc1]

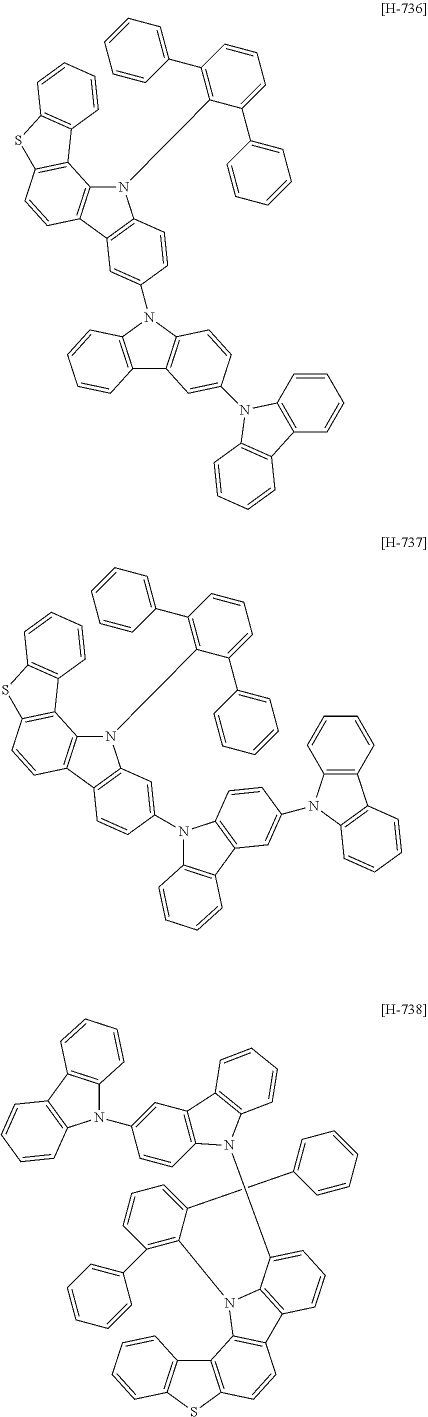 [SMILES: c1ccc(-c2cccc(-c3ccccc3)c2-n2c3ccc(-n4c5ccccc5c5cc(-n6c7ccccc7c7ccccc76)ccc54)cc3c3ccc4sc5ccccc5c4c32)cc1 ; c1ccc(-c2cccc(-c3ccccc3)c2-n2c3cc(-n4c5ccccc5c5cc(-n6c7ccccc7c7ccccc76)ccc54)ccc3c3ccc4sc5ccccc5c4c32)cc1 ; c1ccc(-c2cccc(-c3ccccc3)c2-n2c3c(-n4c5ccccc5c5cc(-n6c7ccccc7c7ccccc76)ccc54)cccc3c3ccc4sc5ccccc5c4c32)cc1]

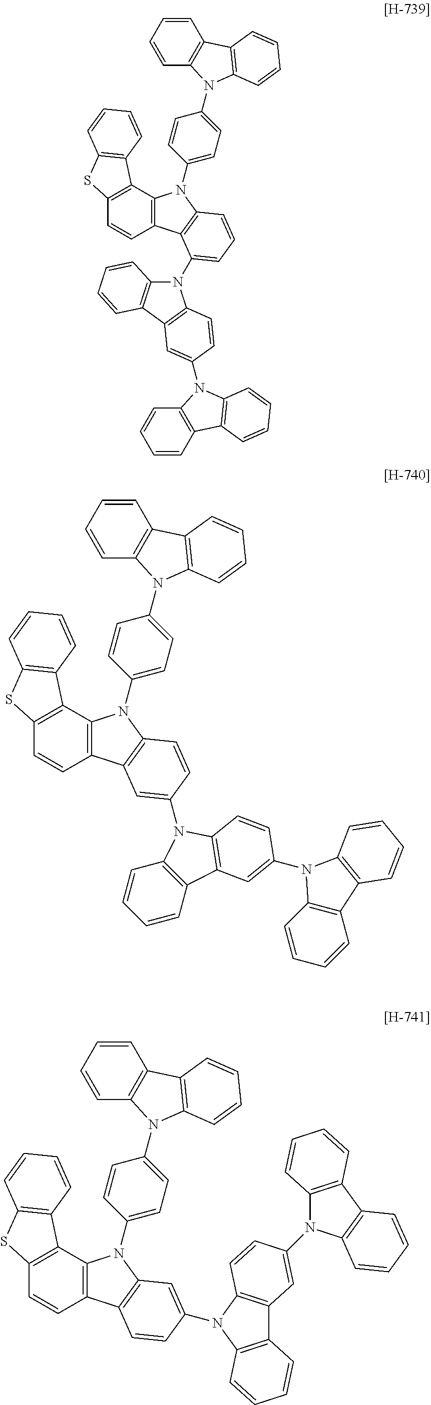 [SMILES: c1ccc2c(c1)sc1ccc3c4c(-n5c6ccccc6c6cc(-n7c8ccccc8c8ccccc87)ccc65)cccc4n(-c4ccc(-n5c6ccccc6c6ccccc65)cc4)c3c12 ; c1ccc2c(c1)sc1ccc3c4cc(-n5c6ccccc6c6cc(-n7c8ccccc8c8ccccc87)ccc65)ccc4n(-c4ccc(-n5c6ccccc6c6ccccc65)cc4)c3c12 ; c1ccc2c(c1)sc1ccc3c4ccc(-n5c6ccccc6c6cc(-n7c8ccccc8c8ccccc87)ccc65)cc4n(-c4ccc(-n5c6ccccc6c6ccccc65)cc4)c3c12]

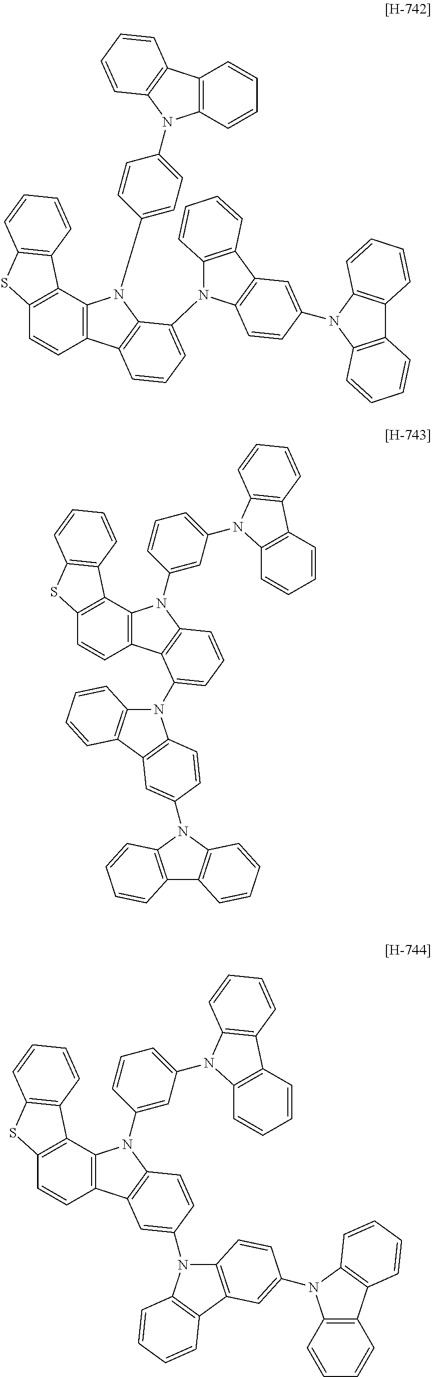 [SMILES: c1ccc2c(c1)sc1ccc3c4cccc(-n5c6ccccc6c6cc(-n7c8ccccc8c8ccccc87)ccc65)c4n(-c4ccc(-n5c6ccccc6c6ccccc65)cc4)c3c12 ; c1cc(-n2c3ccccc3c3ccccc32)cc(-n2c3cccc(-n4c5ccccc5c5cc(-n6c7ccccc7c7ccccc76)ccc54)c3c3ccc4sc5ccccc5c4c32)c1 ; c1cc(-n2c3ccccc3c3ccccc32)cc(-n2c3ccc(-n4c5ccccc5c5cc(-n6c7ccccc7c7ccccc76)ccc54)cc3c3ccc4sc5ccccc5c4c32)c1]

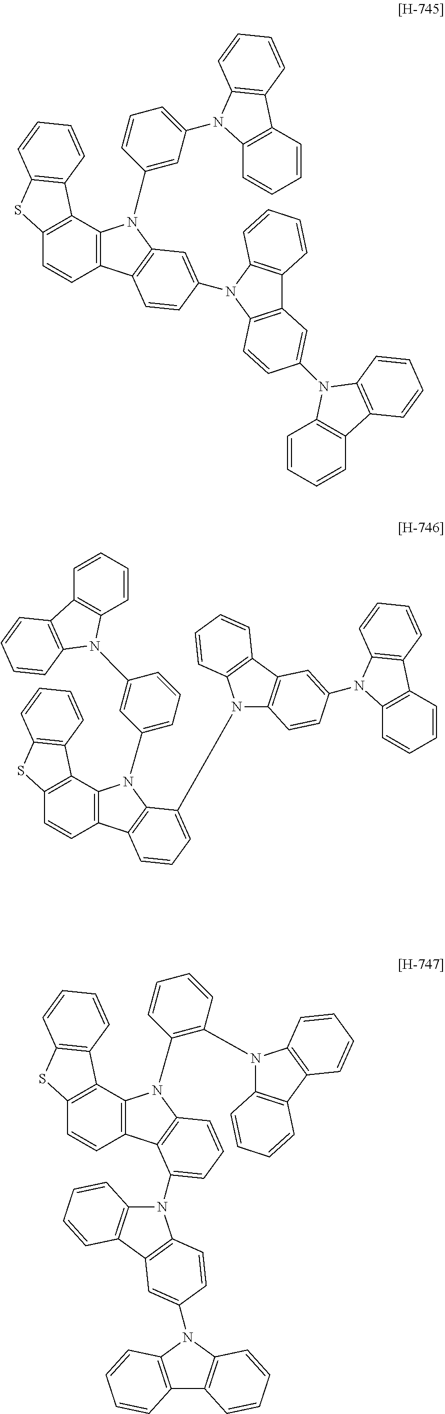 [SMILES: c1cc(-n2c3ccccc3c3ccccc32)cc(-n2c3cc(-n4c5ccccc5c5cc(-n6c7ccccc7c7ccccc76)ccc54)ccc3c3ccc4sc5ccccc5c4c32)c1 ; c1cc(-n2c3ccccc3c3ccccc32)cc(-n2c3c(-n4c5ccccc5c5cc(-n6c7ccccc7c7ccccc76)ccc54)cccc3c3ccc4sc5ccccc5c4c32)c1 ; c1ccc(-n2c3cccc(-n4c5ccccc5c5cc(-n6c7ccccc7c7ccccc76)ccc54)c3c3ccc4sc5ccccc5c4c32)c(-n2c3ccccc3c3ccccc32)c1]

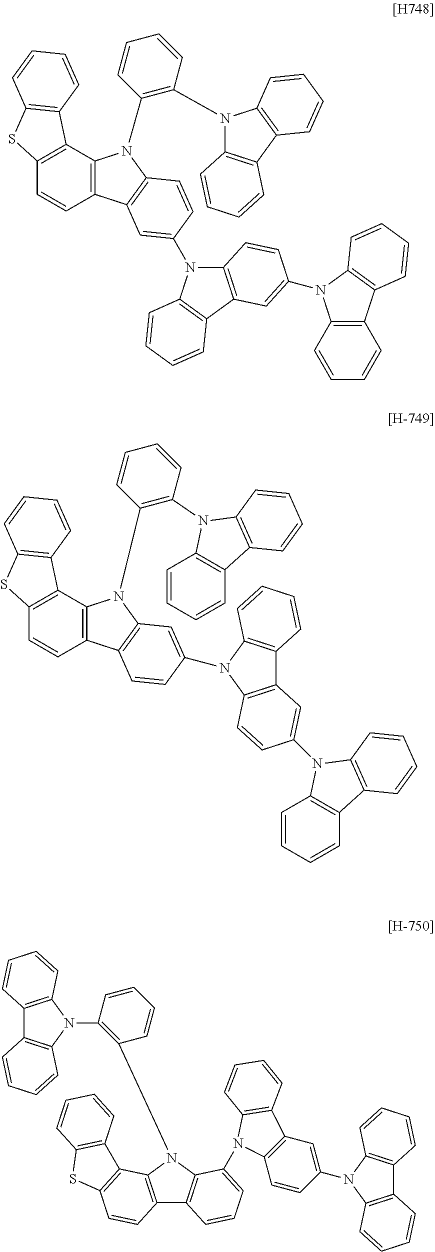 [SMILES: c1ccc(-n2c3ccc(-n4c5ccccc5c5cc(-n6c7ccccc7c7ccccc76)ccc54)cc3c3ccc4sc5ccccc5c4c32)c(-n2c3ccccc3c3ccccc32)c1 ; c1ccc(-n2c3cc(-n4c5ccccc5c5cc(-n6c7ccccc7c7ccccc76)ccc54)ccc3c3ccc4sc5ccccc5c4c32)c(-n2c3ccccc3c3ccccc32)c1 ; c1ccc(-n2c3c(-n4c5ccccc5c5cc(-n6c7ccccc7c7ccccc76)ccc54)cccc3c3ccc4sc5ccccc5c4c32)c(-n2c3ccccc3c3ccccc32)c1]

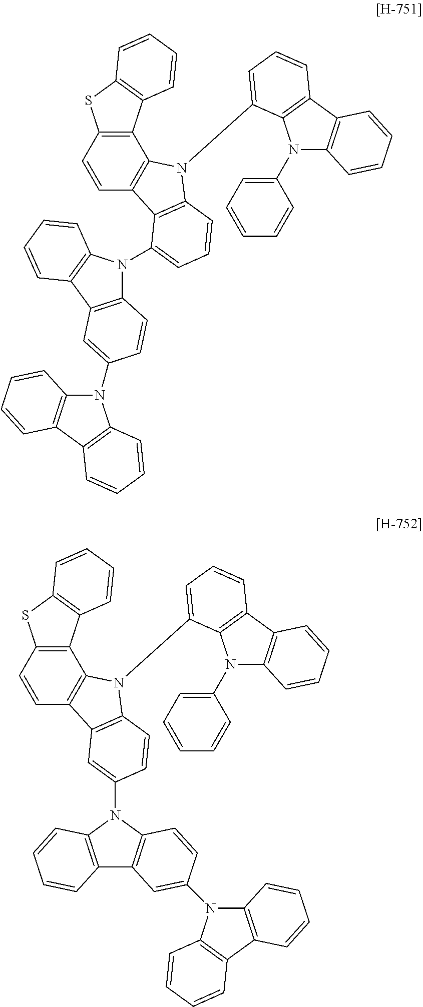 [SMILES: c1ccc(-n2c3ccccc3c3cccc(-n4c5cccc(-n6c7ccccc7c7cc(-n8c9ccccc9c9ccccc98)ccc76)c5c5ccc6sc7ccccc7c6c54)c32)cc1 ; c1ccc(-n2c3ccccc3c3cccc(-n4c5ccc(-n6c7ccccc7c7cc(-n8c9ccccc9c9ccccc98)ccc76)cc5c5ccc6sc7ccccc7c6c54)c32)cc1]

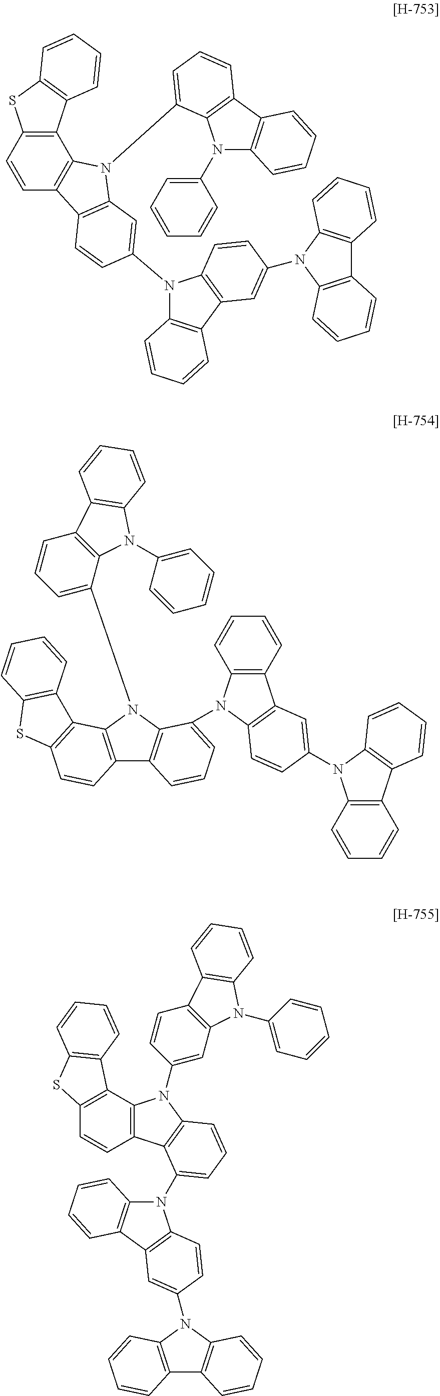 [SMILES: c1ccc(-n2c3ccccc3c3cccc(-n4c5cc(-n6c7ccccc7c7cc(-n8c9ccccc9c9ccccc98)ccc76)ccc5c5ccc6sc7ccccc7c6c54)c32)cc1 ; c1ccc(-n2c3ccccc3c3cccc(-n4c5c(-n6c7ccccc7c7cc(-n8c9ccccc9c9ccccc98)ccc76)cccc5c5ccc6sc7ccccc7c6c54)c32)cc1 ; c1ccc(-n2c3ccccc3c3ccc(-n4c5cccc(-n6c7ccccc7c7cc(-n8c9ccccc9c9ccccc98)ccc76)c5c5ccc6sc7ccccc7c6c54)cc32)cc1]

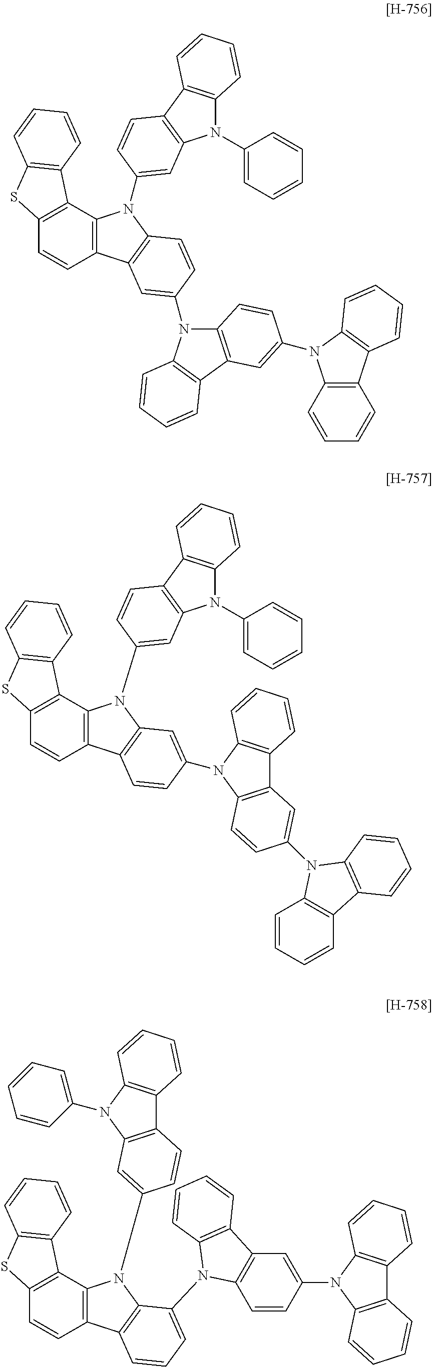 [SMILES: c1ccc(-n2c3ccccc3c3ccc(-n4c5ccc(-n6c7ccccc7c7cc(-n8c9ccccc9c9ccccc98)ccc76)cc5c5ccc6sc7ccccc7c6c54)cc32)cc1 ; c1ccc(-n2c3ccccc3c3ccc(-n4c5cc(-n6c7ccccc7c7cc(-n8c9ccccc9c9ccccc98)ccc76)ccc5c5ccc6sc7ccccc7c6c54)cc32)cc1 ; c1ccc(-n2c3ccccc3c3ccc(-n4c5c(-n6c7ccccc7c7cc(-n8c9ccccc9c9ccccc98)ccc76)cccc5c5ccc6sc7ccccc7c6c54)cc32)cc1]

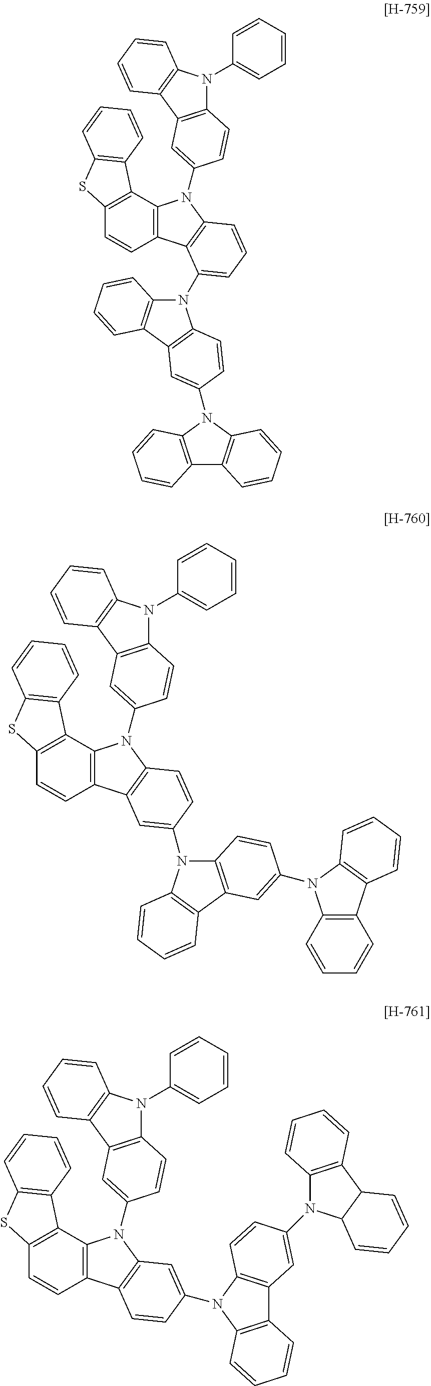 [SMILES: c1ccc(-n2c3ccccc3c3cc(-n4c5cccc(-n6c7ccccc7c7cc(-n8c9ccccc9c9ccccc98)ccc76)c5c5ccc6sc7ccccc7c6c54)ccc32)cc1 ; c1ccc(-n2c3ccccc3c3cc(-n4c5ccc(-n6c7ccccc7c7cc(-n8c9ccccc9c9ccccc98)ccc76)cc5c5ccc6sc7ccccc7c6c54)ccc32)cc1 ; C1=CC2c3ccccc3N(c3ccc4c(c3)c3ccccc3n4-c3ccc4c5ccc6sc7ccccc7c6c5n(-c5ccc6c(c5)c5ccccc5n6-c5ccccc5)c4c3)C2C=C1]

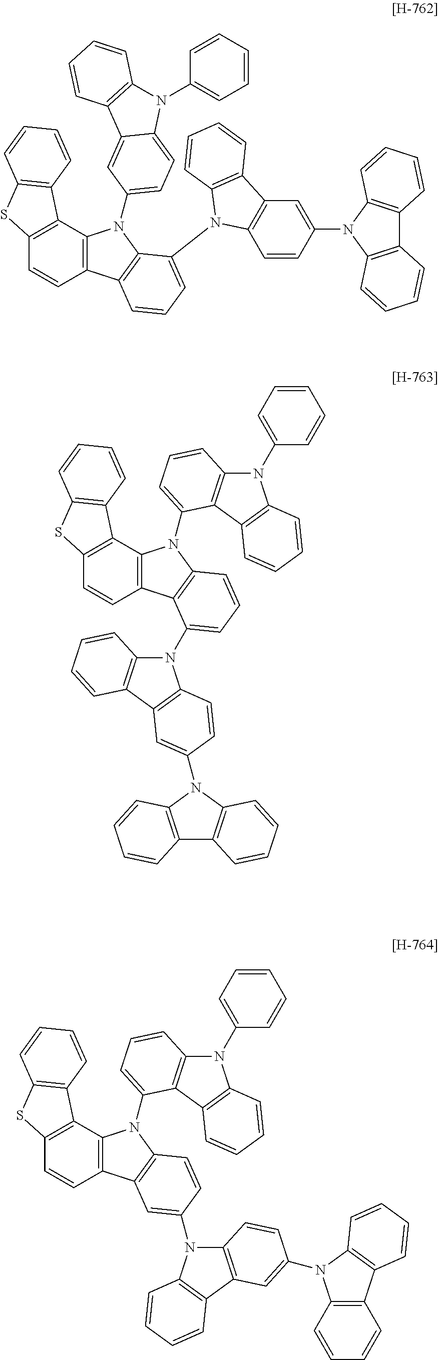 [SMILES: c1ccc(-n2c3ccccc3c3cc(-n4c5c(-n6c7ccccc7c7cc(-n8c9ccccc9c9ccccc98)ccc76)cccc5c5ccc6sc7ccccc7c6c54)ccc32)cc1 ; c1ccc(-n2c3ccccc3c3c(-n4c5cccc(-n6c7ccccc7c7cc(-n8c9ccccc9c9ccccc98)ccc76)c5c5ccc6sc7ccccc7c6c54)cccc32)cc1 ; c1ccc(-n2c3ccccc3c3c(-n4c5ccc(-n6c7ccccc7c7cc(-n8c9ccccc9c9ccccc98)ccc76)cc5c5ccc6sc7ccccc7c6c54)cccc32)cc1]

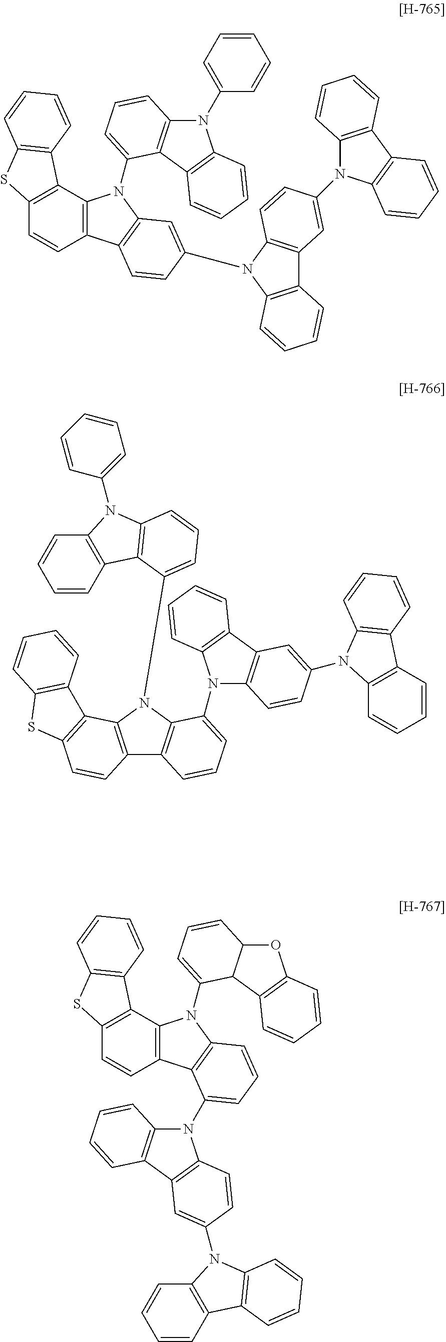 [SMILES: c1ccc(-n2c3ccccc3c3c(-n4c5cc(-n6c7ccccc7c7cc(-n8c9ccccc9c9ccccc98)ccc76)ccc5c5ccc6sc7ccccc7c6c54)cccc32)cc1 ; c1ccc(-n2c3ccccc3c3c(-n4c5c(-n6c7ccccc7c7cc(-n8c9ccccc9c9ccccc98)ccc76)cccc5c5ccc6sc7ccccc7c6c54)cccc32)cc1 ; C1=CC2Oc3ccccc3C2C(n2c3cccc(-n4c5ccccc5c5cc(-n6c7ccccc7c7ccccc76)ccc54)c3c3ccc4sc5ccccc5c4c32)=C1]

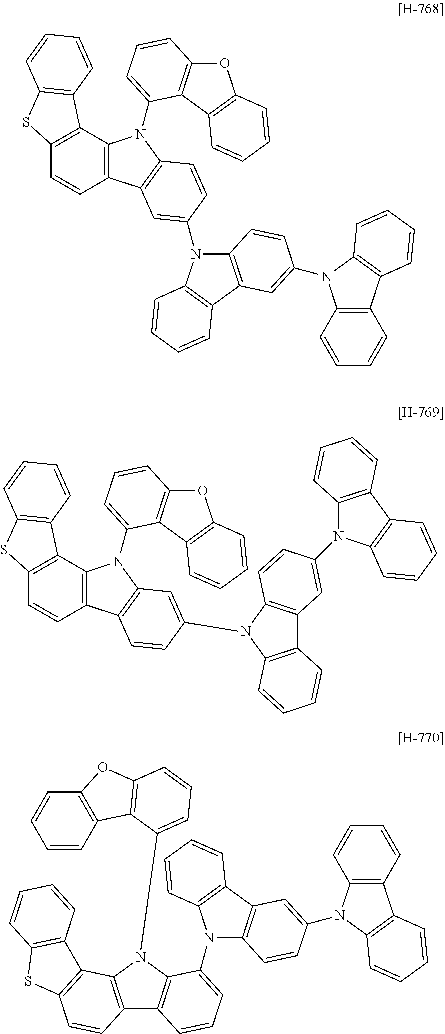 [SMILES: c1ccc2c(c1)oc1cccc(-n3c4ccc(-n5c6ccccc6c6cc(-n7c8ccccc8c8ccccc87)ccc65)cc4c4ccc5sc6ccccc6c5c43)c12 ; c1ccc2c(c1)oc1cccc(-n3c4cc(-n5c6ccccc6c6cc(-n7c8ccccc8c8ccccc87)ccc65)ccc4c4ccc5sc6ccccc6c5c43)c12 ; c1ccc2c(c1)oc1cccc(-n3c4c(-n5c6ccccc6c6cc(-n7c8ccccc8c8ccccc87)ccc65)cccc4c4ccc5sc6ccccc6c5c43)c12]

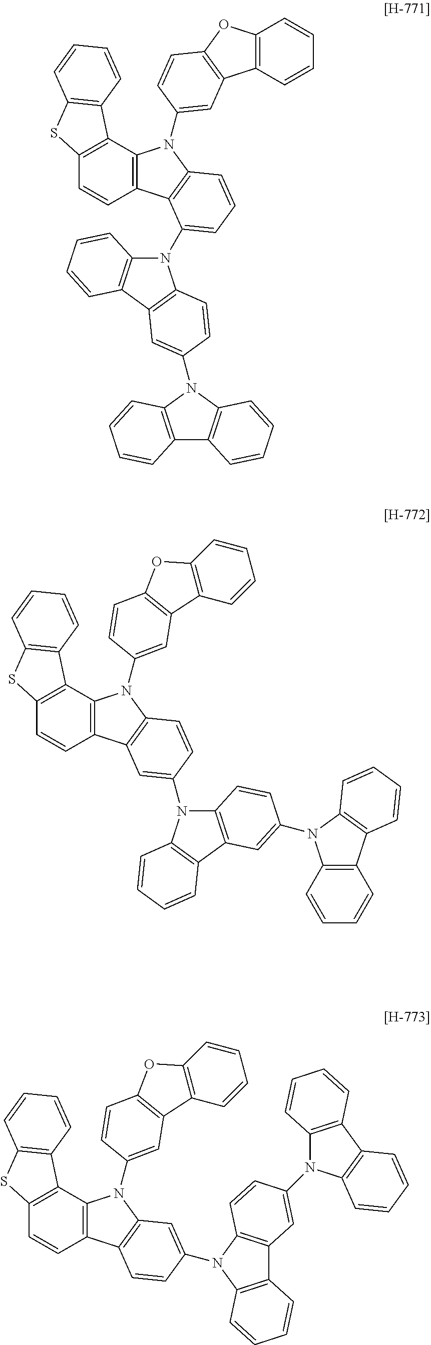 [SMILES: c1ccc2c(c1)oc1ccc(-n3c4cccc(-n5c6ccccc6c6cc(-n7c8ccccc8c8ccccc87)ccc65)c4c4ccc5sc6ccccc6c5c43)cc12 ; c1ccc2c(c1)oc1ccc(-n3c4ccc(-n5c6ccccc6c6cc(-n7c8ccccc8c8ccccc87)ccc65)cc4c4ccc5sc6ccccc6c5c43)cc12 ; c1ccc2c(c1)oc1ccc(-n3c4cc(-n5c6ccccc6c6cc(-n7c8ccccc8c8ccccc87)ccc65)ccc4c4ccc5sc6ccccc6c5c43)cc12]

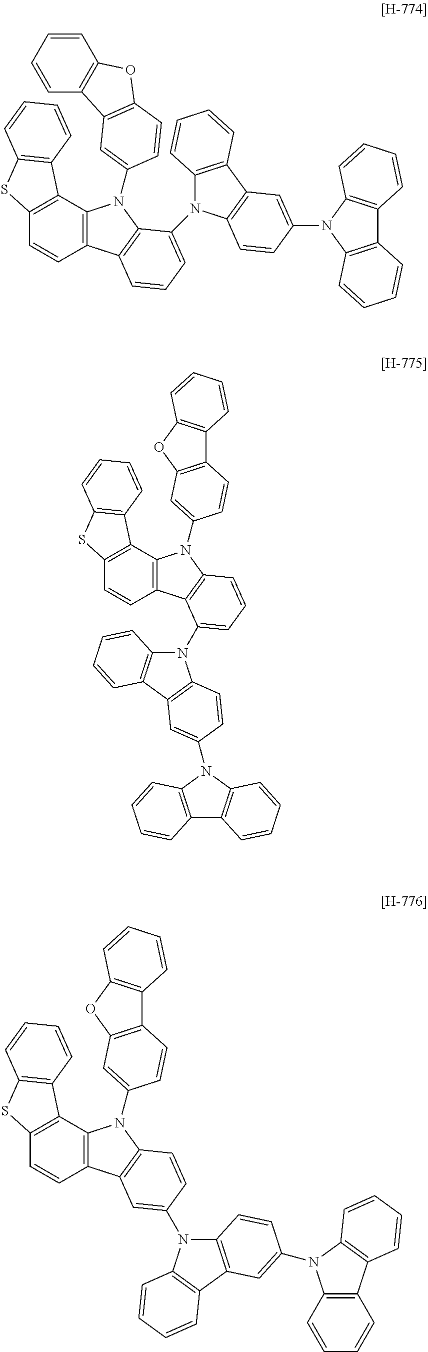 [SMILES: c1ccc2c(c1)oc1ccc(-n3c4c(-n5c6ccccc6c6cc(-n7c8ccccc8c8ccccc87)ccc65)cccc4c4ccc5sc6ccccc6c5c43)cc12 ; c1ccc2c(c1)oc1cc(-n3c4cccc(-n5c6ccccc6c6cc(-n7c8ccccc8c8ccccc87)ccc65)c4c4ccc5sc6ccccc6c5c43)ccc12 ; c1ccc2c(c1)oc1cc(-n3c4ccc(-n5c6ccccc6c6cc(-n7c8ccccc8c8ccccc87)ccc65)cc4c4ccc5sc6ccccc6c5c43)ccc12]

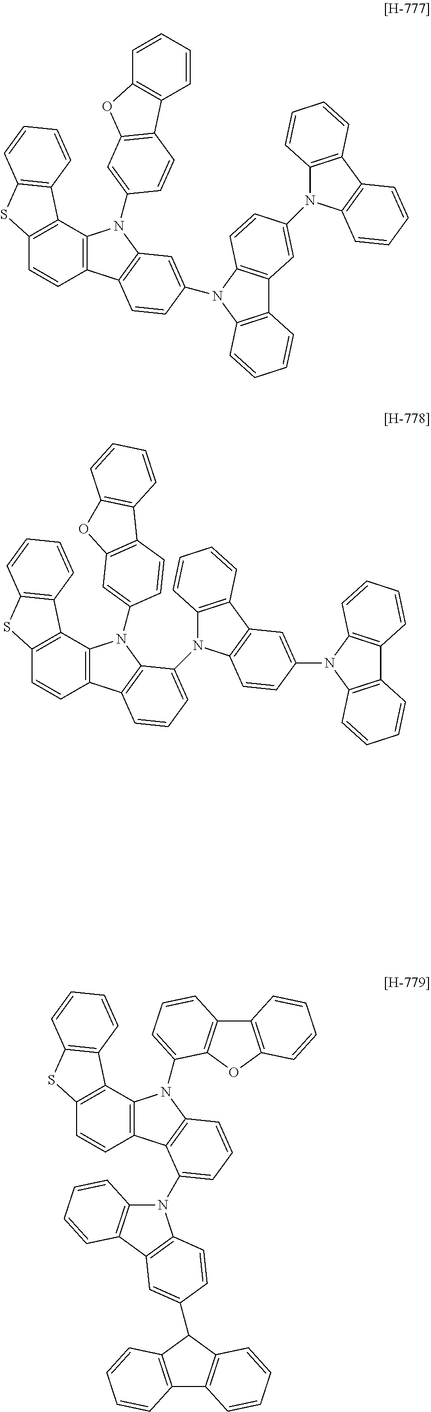 [SMILES: c1ccc2c(c1)oc1cc(-n3c4cc(-n5c6ccccc6c6cc(-n7c8ccccc8c8ccccc87)ccc65)ccc4c4ccc5sc6ccccc6c5c43)ccc12 ; c1ccc2c(c1)oc1cc(-n3c4c(-n5c6ccccc6c6cc(-n7c8ccccc8c8ccccc87)ccc65)cccc4c4ccc5sc6ccccc6c5c43)ccc12 ; c1ccc2c(c1)-c1ccccc1C2c1ccc2c(c1)c1ccccc1n2-c1cccc2c1c1ccc3sc4ccccc4c3c1n2-c1cccc2c1oc1ccccc12]

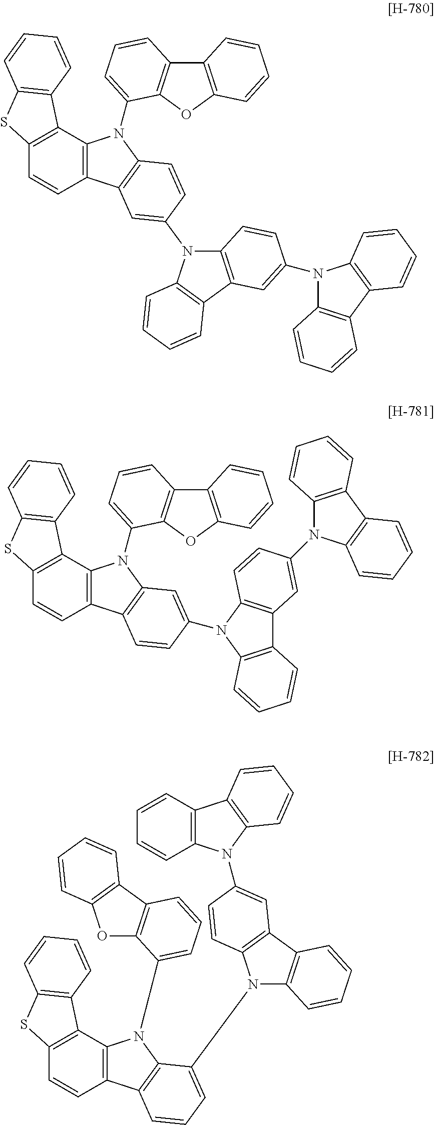 [SMILES: c1ccc2c(c1)oc1c(-n3c4ccc(-n5c6ccccc6c6cc(-n7c8ccccc8c8ccccc87)ccc65)cc4c4ccc5sc6ccccc6c5c43)cccc12 ; c1ccc2c(c1)oc1c(-n3c4cc(-n5c6ccccc6c6cc(-n7c8ccccc8c8ccccc87)ccc65)ccc4c4ccc5sc6ccccc6c5c43)cccc12 ; c1ccc2c(c1)oc1c(-n3c4c(-n5c6ccccc6c6cc(-n7c8ccccc8c8ccccc87)ccc65)cccc4c4ccc5sc6ccccc6c5c43)cccc12]

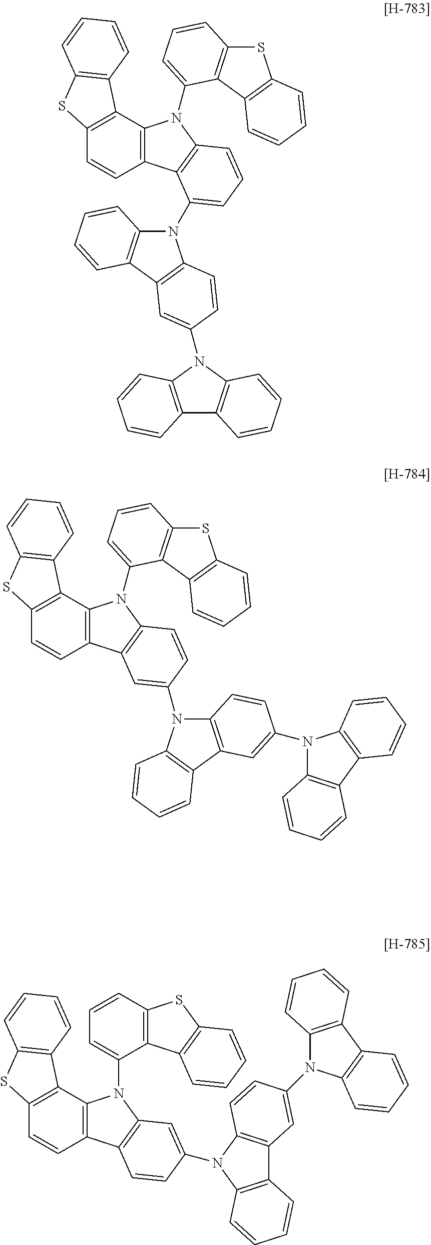 [SMILES: c1ccc2c(c1)sc1cccc(-n3c4cccc(-n5c6ccccc6c6cc(-n7c8ccccc8c8ccccc87)ccc65)c4c4ccc5sc6ccccc6c5c43)c12 ; c1ccc2c(c1)sc1cccc(-n3c4ccc(-n5c6ccccc6c6cc(-n7c8ccccc8c8ccccc87)ccc65)cc4c4ccc5sc6ccccc6c5c43)c12 ; c1ccc2c(c1)sc1cccc(-n3c4cc(-n5c6ccccc6c6cc(-n7c8ccccc8c8ccccc87)ccc65)ccc4c4ccc5sc6ccccc6c5c43)c12]

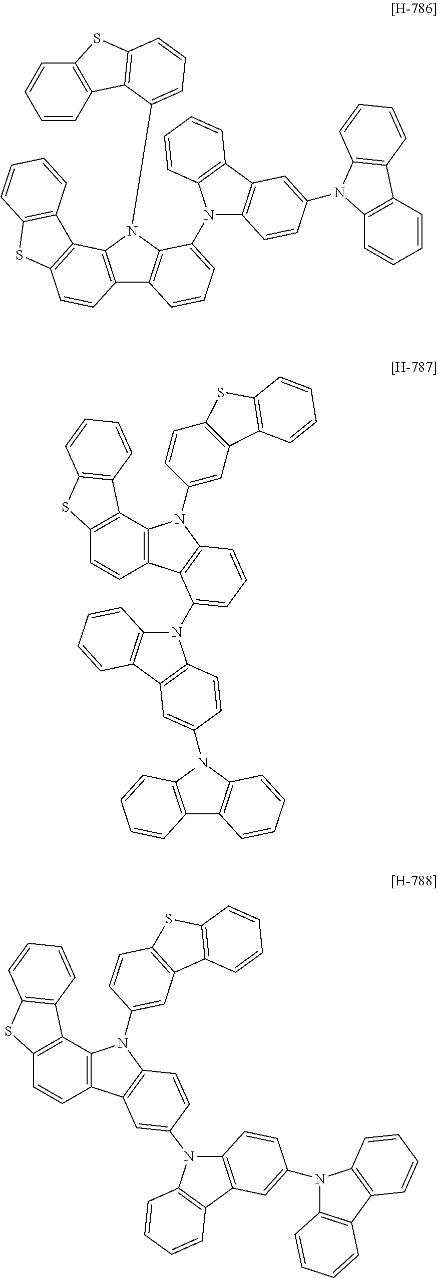 [SMILES: c1ccc2c(c1)sc1cccc(-n3c4c(-n5c6ccccc6c6cc(-n7c8ccccc8c8ccccc87)ccc65)cccc4c4ccc5sc6ccccc6c5c43)c12 ; c1ccc2c(c1)sc1ccc(-n3c4cccc(-n5c6ccccc6c6cc(-n7c8ccccc8c8ccccc87)ccc65)c4c4ccc5sc6ccccc6c5c43)cc12 ; c1ccc2c(c1)sc1ccc(-n3c4ccc(-n5c6ccccc6c6cc(-n7c8ccccc8c8ccccc87)ccc65)cc4c4ccc5sc6ccccc6c5c43)cc12]

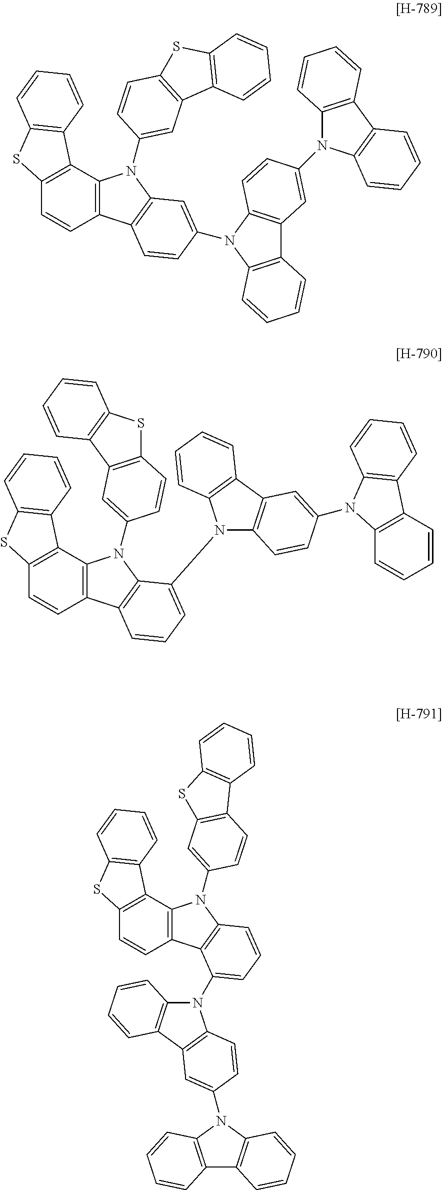 [SMILES: c1ccc2c(c1)sc1ccc(-n3c4cc(-n5c6ccccc6c6cc(-n7c8ccccc8c8ccccc87)ccc65)ccc4c4ccc5sc6ccccc6c5c43)cc12 ; c1ccc2c(c1)sc1ccc(-n3c4c(-n5c6ccccc6c6cc(-n7c8ccccc8c8ccccc87)ccc65)cccc4c4ccc5sc6ccccc6c5c43)cc12 ; c1ccc2c(c1)sc1cc(-n3c4cccc(-n5c6ccccc6c6cc(-n7c8ccccc8c8ccccc87)ccc65)c4c4ccc5sc6ccccc6c5c43)ccc12]

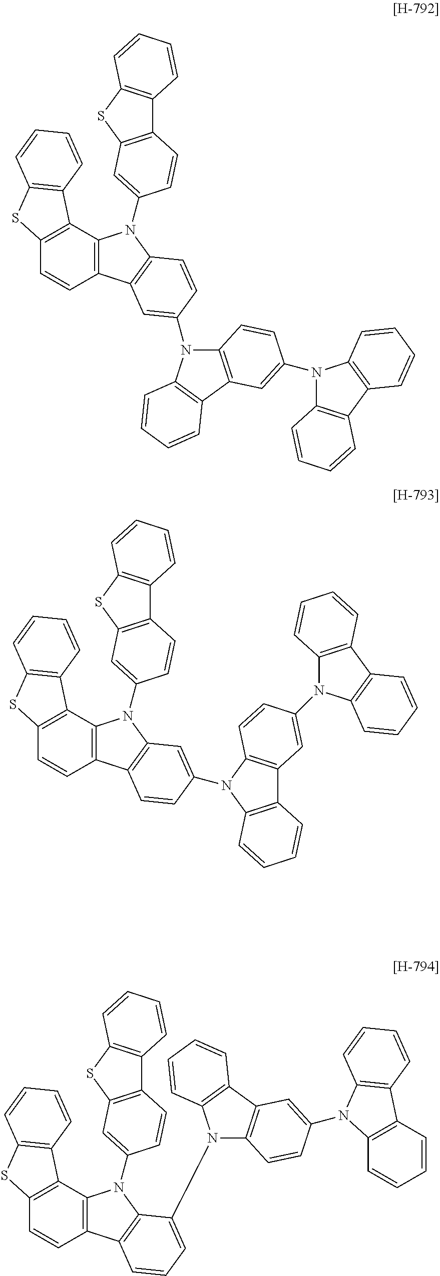 [SMILES: c1ccc2c(c1)sc1cc(-n3c4ccc(-n5c6ccccc6c6cc(-n7c8ccccc8c8ccccc87)ccc65)cc4c4ccc5sc6ccccc6c5c43)ccc12 ; c1ccc2c(c1)sc1cc(-n3c4cc(-n5c6ccccc6c6cc(-n7c8ccccc8c8ccccc87)ccc65)ccc4c4ccc5sc6ccccc6c5c43)ccc12 ; c1ccc2c(c1)sc1cc(-n3c4c(-n5c6ccccc6c6cc(-n7c8ccccc8c8ccccc87)ccc65)cccc4c4ccc5sc6ccccc6c5c43)ccc12]

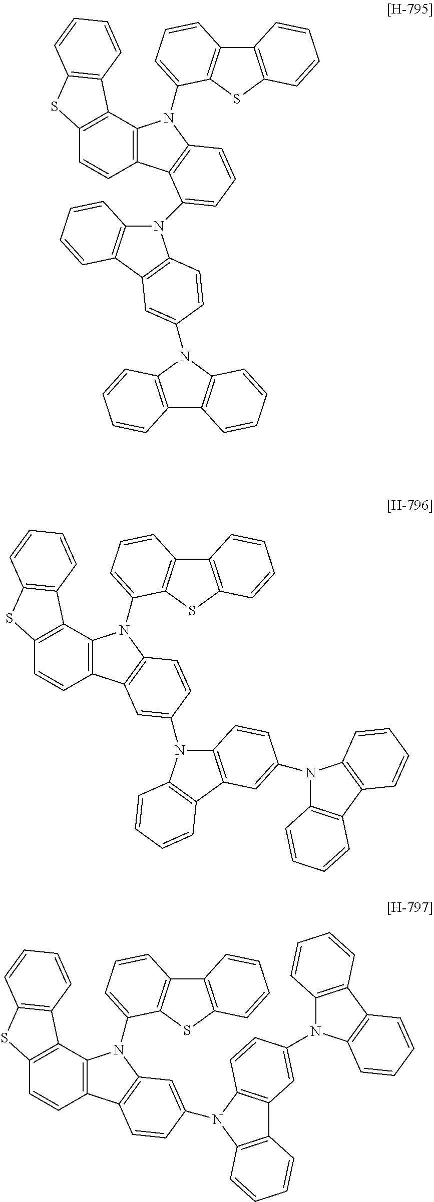 [SMILES: c1ccc2c(c1)sc1c(-n3c4cccc(-n5c6ccccc6c6cc(-n7c8ccccc8c8ccccc87)ccc65)c4c4ccc5sc6ccccc6c5c43)cccc12 ; c1ccc2c(c1)sc1c(-n3c4ccc(-n5c6ccccc6c6cc(-n7c8ccccc8c8ccccc87)ccc65)cc4c4ccc5sc6ccccc6c5c43)cccc12 ; c1ccc2c(c1)sc1c(-n3c4cc(-n5c6ccccc6c6cc(-n7c8ccccc8c8ccccc87)ccc65)ccc4c4ccc5sc6ccccc6c5c43)cccc12]

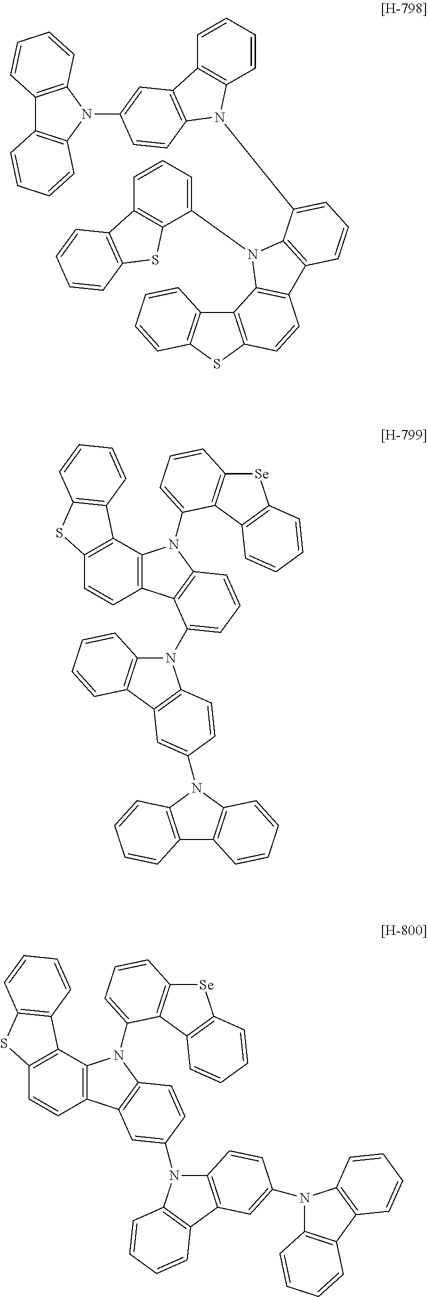 [SMILES: c1ccc2c(c1)sc1c(-n3c4c(-n5c6ccccc6c6cc(-n7c8ccccc8c8ccccc87)ccc65)cccc4c4ccc5sc6ccccc6c5c43)cccc12 ; c1ccc2c(c1)sc1ccc3c4c(-n5c6ccccc6c6cc(-n7c8ccccc8c8ccccc87)ccc65)cccc4n(-c4cccc5[se]c6ccccc6c45)c3c12 ; c1ccc2c(c1)sc1ccc3c4cc(-n5c6ccccc6c6cc(-n7c8ccccc8c8ccccc87)ccc65)ccc4n(-c4cccc5[se]c6ccccc6c45)c3c12]

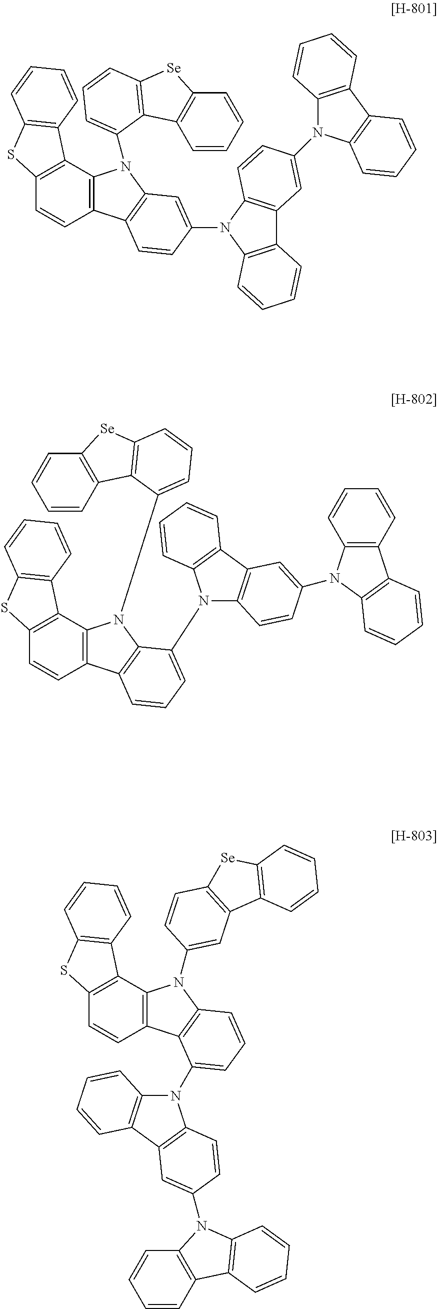 [SMILES: c1ccc2c(c1)sc1ccc3c4ccc(-n5c6ccccc6c6cc(-n7c8ccccc8c8ccccc87)ccc65)cc4n(-c4cccc5[se]c6ccccc6c45)c3c12 ; c1ccc2c(c1)sc1ccc3c4cccc(-n5c6ccccc6c6cc(-n7c8ccccc8c8ccccc87)ccc65)c4n(-c4cccc5[se]c6ccccc6c45)c3c12 ; c1ccc2c(c1)sc1ccc3c4c(-n5c6ccccc6c6cc(-n7c8ccccc8c8ccccc87)ccc65)cccc4n(-c4ccc5[se]c6ccccc6c5c4)c3c12]

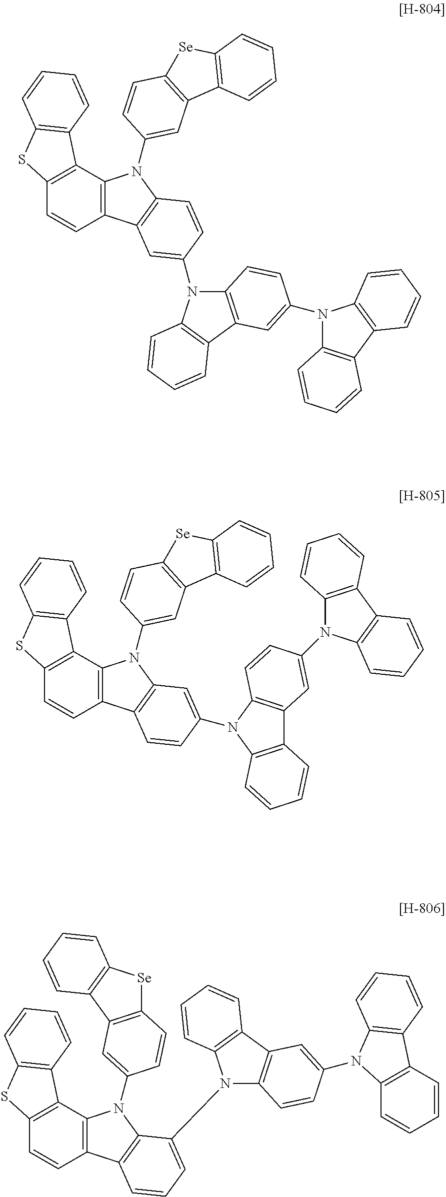 [SMILES: c1ccc2c(c1)sc1ccc3c4cc(-n5c6ccccc6c6cc(-n7c8ccccc8c8ccccc87)ccc65)ccc4n(-c4ccc5[se]c6ccccc6c5c4)c3c12 ; c1ccc2c(c1)sc1ccc3c4ccc(-n5c6ccccc6c6cc(-n7c8ccccc8c8ccccc87)ccc65)cc4n(-c4ccc5[se]c6ccccc6c5c4)c3c12 ; c1ccc2c(c1)sc1ccc3c4cccc(-n5c6ccccc6c6cc(-n7c8ccccc8c8ccccc87)ccc65)c4n(-c4ccc5[se]c6ccccc6c5c4)c3c12]

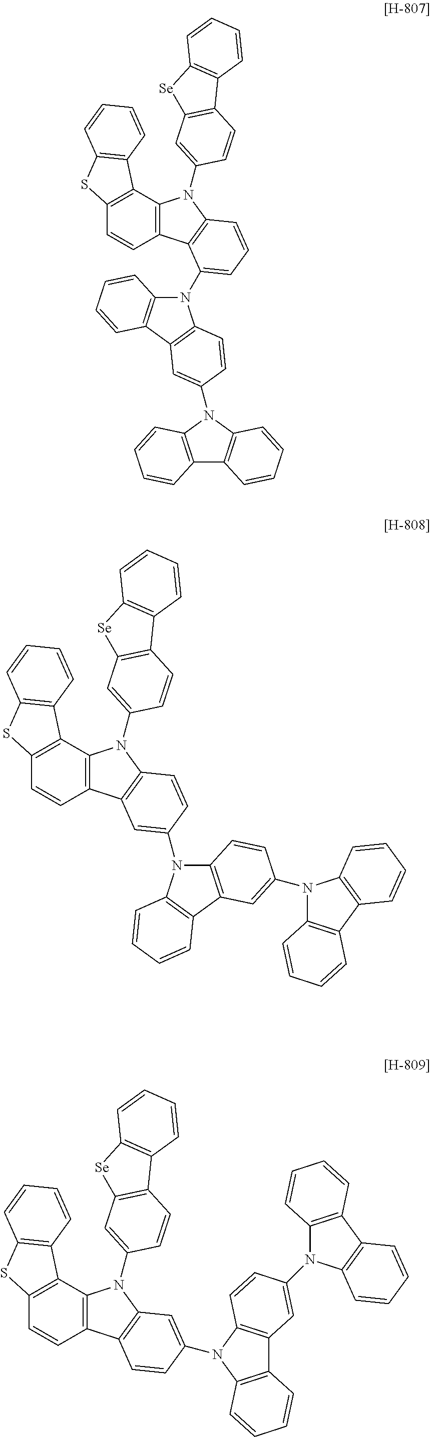 [SMILES: c1ccc2c(c1)sc1ccc3c4c(-n5c6ccccc6c6cc(-n7c8ccccc8c8ccccc87)ccc65)cccc4n(-c4ccc5c(c4)[se]c4ccccc45)c3c12 ; c1ccc2c(c1)sc1ccc3c4cc(-n5c6ccccc6c6cc(-n7c8ccccc8c8ccccc87)ccc65)ccc4n(-c4ccc5c(c4)[se]c4ccccc45)c3c12 ; c1ccc2c(c1)sc1ccc3c4ccc(-n5c6ccccc6c6cc(-n7c8ccccc8c8ccccc87)ccc65)cc4n(-c4ccc5c(c4)[se]c4ccccc45)c3c12]

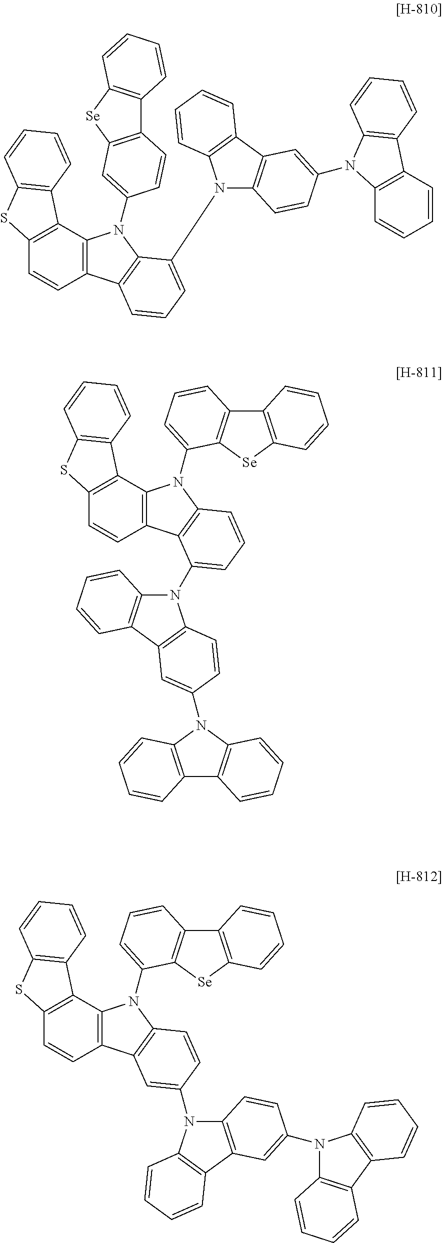 [SMILES: c1ccc2c(c1)sc1ccc3c4cccc(-n5c6ccccc6c6cc(-n7c8ccccc8c8ccccc87)ccc65)c4n(-c4ccc5c(c4)[se]c4ccccc45)c3c12 ; c1ccc2c(c1)sc1ccc3c4c(-n5c6ccccc6c6cc(-n7c8ccccc8c8ccccc87)ccc65)cccc4n(-c4cccc5c4[se]c4ccccc45)c3c12 ; c1ccc2c(c1)sc1ccc3c4cc(-n5c6ccccc6c6cc(-n7c8ccccc8c8ccccc87)ccc65)ccc4n(-c4cccc5c4[se]c4ccccc45)c3c12]

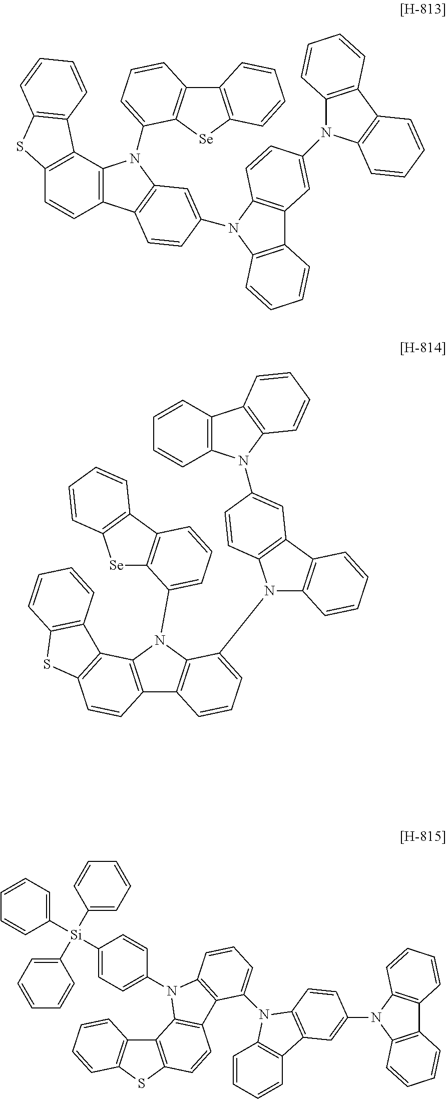 [SMILES: c1ccc2c(c1)sc1ccc3c4ccc(-n5c6ccccc6c6cc(-n7c8ccccc8c8ccccc87)ccc65)cc4n(-c4cccc5c4[se]c4ccccc45)c3c12 ; c1ccc2c(c1)sc1ccc3c4cccc(-n5c6ccccc6c6cc(-n7c8ccccc8c8ccccc87)ccc65)c4n(-c4cccc5c4[se]c4ccccc45)c3c12 ; c1ccc([Si](c2ccccc2)(c2ccccc2)c2ccc(-n3c4cccc(-n5c6ccccc6c6cc(-n7c8ccccc8c8ccccc87)ccc65)c4c4ccc5sc6ccccc6c5c43)cc2)cc1]

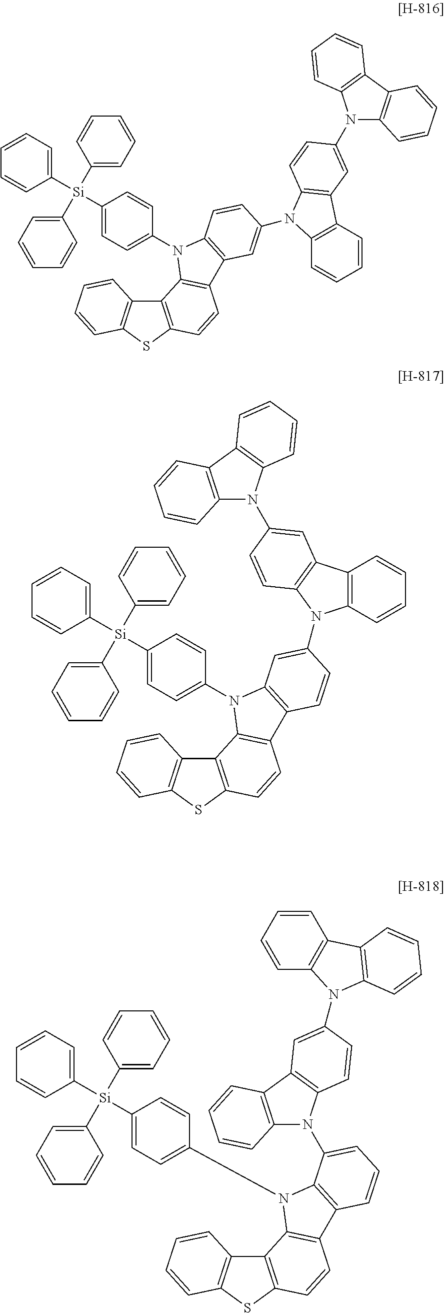 [SMILES: c1ccc([Si](c2ccccc2)(c2ccccc2)c2ccc(-n3c4ccc(-n5c6ccccc6c6cc(-n7c8ccccc8c8ccccc87)ccc65)cc4c4ccc5sc6ccccc6c5c43)cc2)cc1 ; c1ccc([Si](c2ccccc2)(c2ccccc2)c2ccc(-n3c4cc(-n5c6ccccc6c6cc(-n7c8ccccc8c8ccccc87)ccc65)ccc4c4ccc5sc6ccccc6c5c43)cc2)cc1 ; c1ccc([Si](c2ccccc2)(c2ccccc2)c2ccc(-n3c4c(-n5c6ccccc6c6cc(-n7c8ccccc8c8ccccc87)ccc65)cccc4c4ccc5sc6ccccc6c5c43)cc2)cc1]

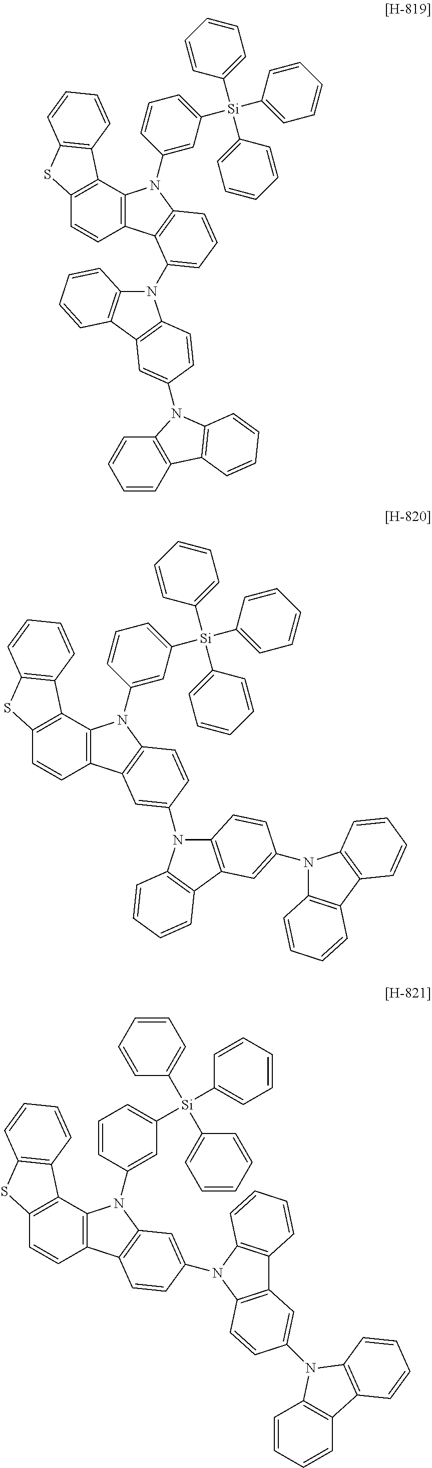 [SMILES: c1ccc([Si](c2ccccc2)(c2ccccc2)c2cccc(-n3c4cccc(-n5c6ccccc6c6cc(-n7c8ccccc8c8ccccc87)ccc65)c4c4ccc5sc6ccccc6c5c43)c2)cc1 ; c1ccc([Si](c2ccccc2)(c2ccccc2)c2cccc(-n3c4ccc(-n5c6ccccc6c6cc(-n7c8ccccc8c8ccccc87)ccc65)cc4c4ccc5sc6ccccc6c5c43)c2)cc1 ; c1ccc([Si](c2ccccc2)(c2ccccc2)c2cccc(-n3c4cc(-n5c6ccccc6c6cc(-n7c8ccccc8c8ccccc87)ccc65)ccc4c4ccc5sc6ccccc6c5c43)c2)cc1]

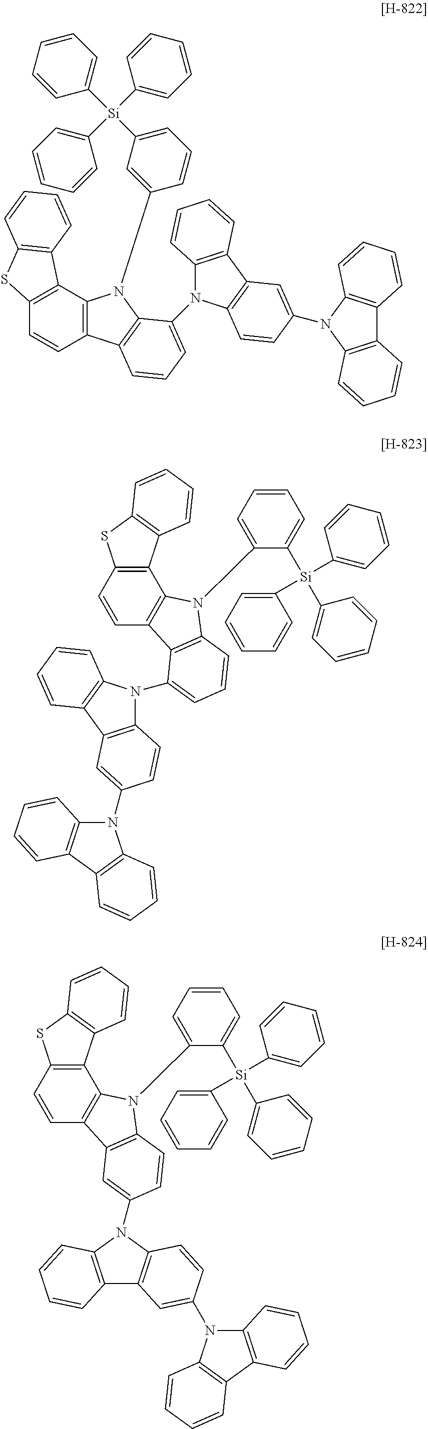 [SMILES: c1ccc([Si](c2ccccc2)(c2ccccc2)c2cccc(-n3c4c(-n5c6ccccc6c6cc(-n7c8ccccc8c8ccccc87)ccc65)cccc4c4ccc5sc6ccccc6c5c43)c2)cc1 ; c1ccc([Si](c2ccccc2)(c2ccccc2)c2ccccc2-n2c3cccc(-n4c5ccccc5c5cc(-n6c7ccccc7c7ccccc76)ccc54)c3c3ccc4sc5ccccc5c4c32)cc1 ; c1ccc([Si](c2ccccc2)(c2ccccc2)c2ccccc2-n2c3ccc(-n4c5ccccc5c5cc(-n6c7ccccc7c7ccccc76)ccc54)cc3c3ccc4sc5ccccc5c4c32)cc1]

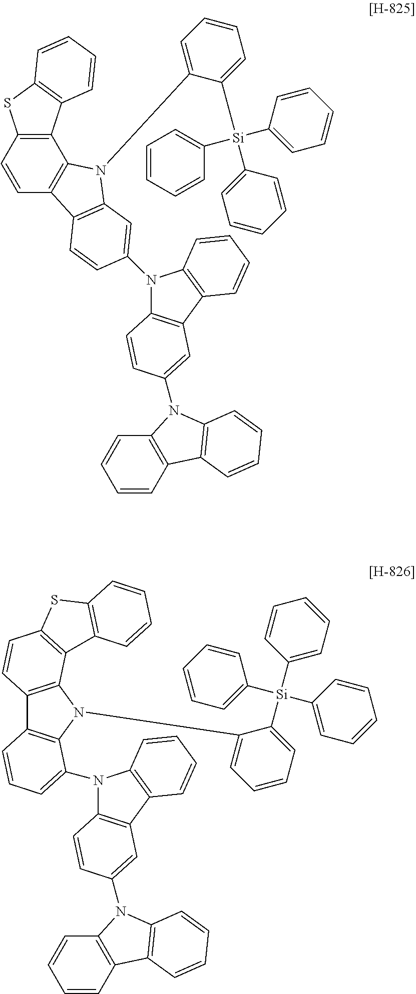 [SMILES: c1ccc([Si](c2ccccc2)(c2ccccc2)c2ccccc2-n2c3cc(-n4c5ccccc5c5cc(-n6c7ccccc7c7ccccc76)ccc54)ccc3c3ccc4sc5ccccc5c4c32)cc1 ; c1ccc([Si](c2ccccc2)(c2ccccc2)c2ccccc2-n2c3c(-n4c5ccccc5c5cc(-n6c7ccccc7c7ccccc76)ccc54)cccc3c3ccc4sc5ccccc5c4c32)cc1]

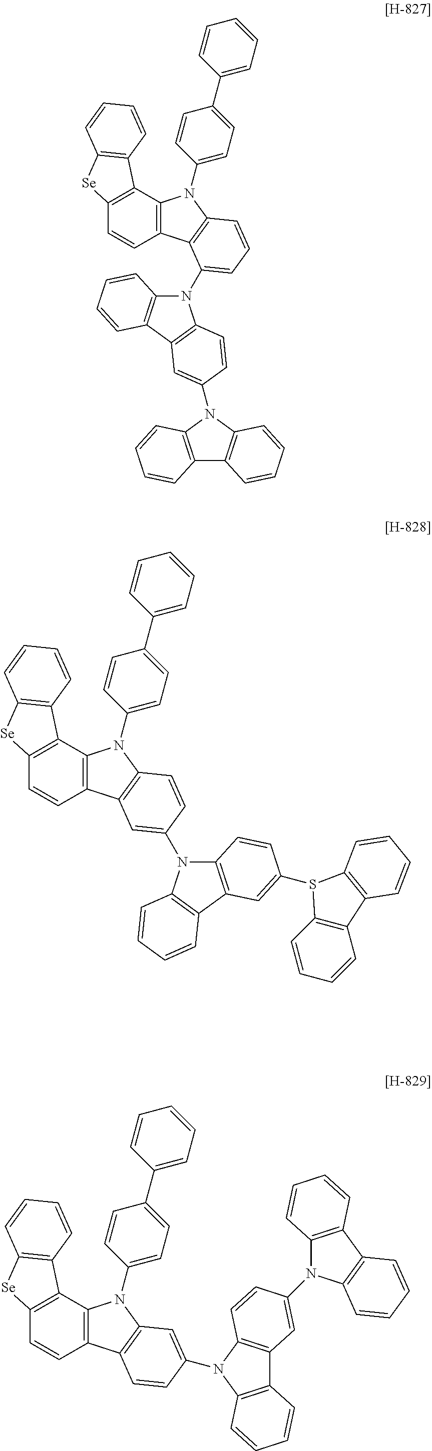

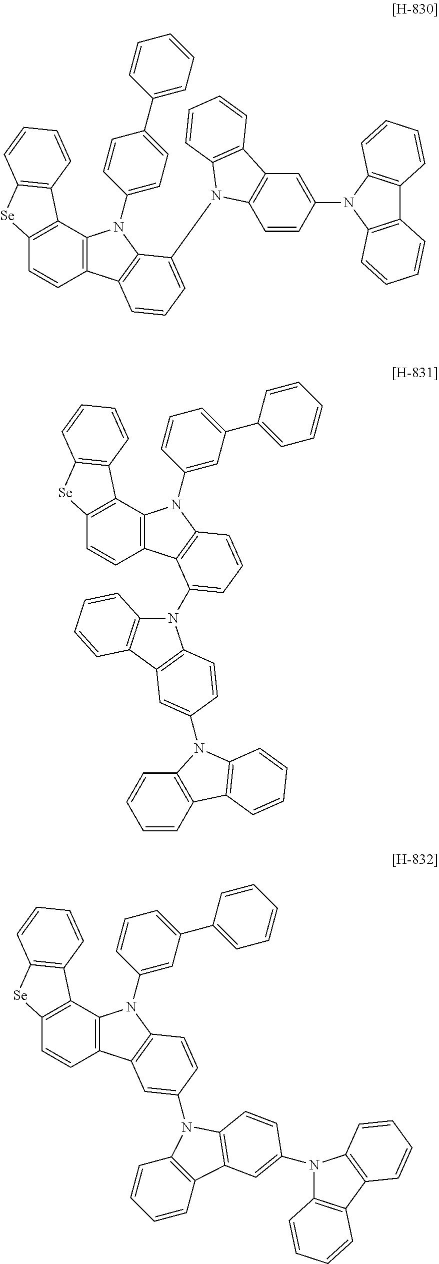 [SMILES: c1ccc(-c2ccc(-n3c4c(-n5c6ccccc6c6cc(-n7c8ccccc8c8ccccc87)ccc65)cccc4c4ccc5[se]c6ccccc6c5c43)cc2)cc1 ; c1ccc(-c2cccc(-n3c4cccc(-n5c6ccccc6c6cc(-n7c8ccccc8c8ccccc87)ccc65)c4c4ccc5[se]c6ccccc6c5c43)c2)cc1 ; c1ccc(-c2cccc(-n3c4ccc(-n5c6ccccc6c6cc(-n7c8ccccc8c8ccccc87)ccc65)cc4c4ccc5[se]c6ccccc6c5c43)c2)cc1]

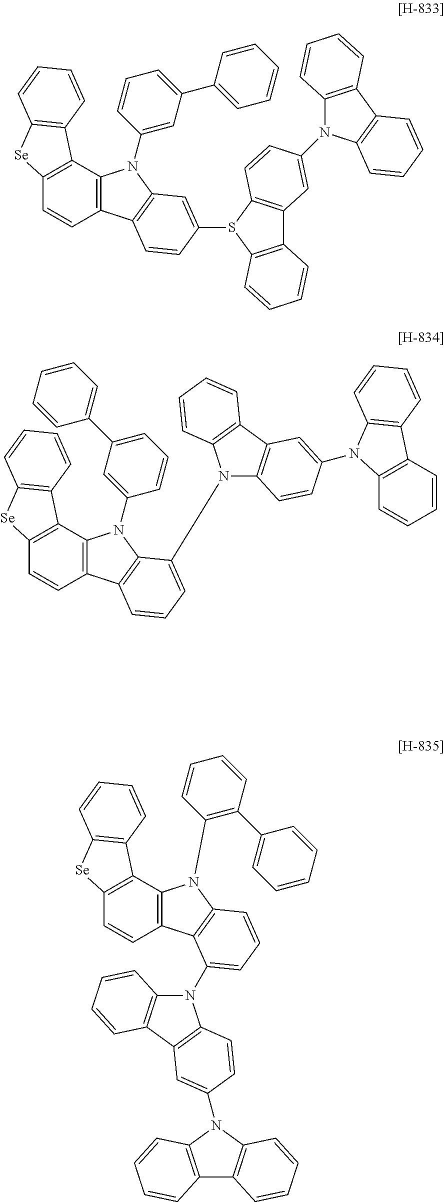

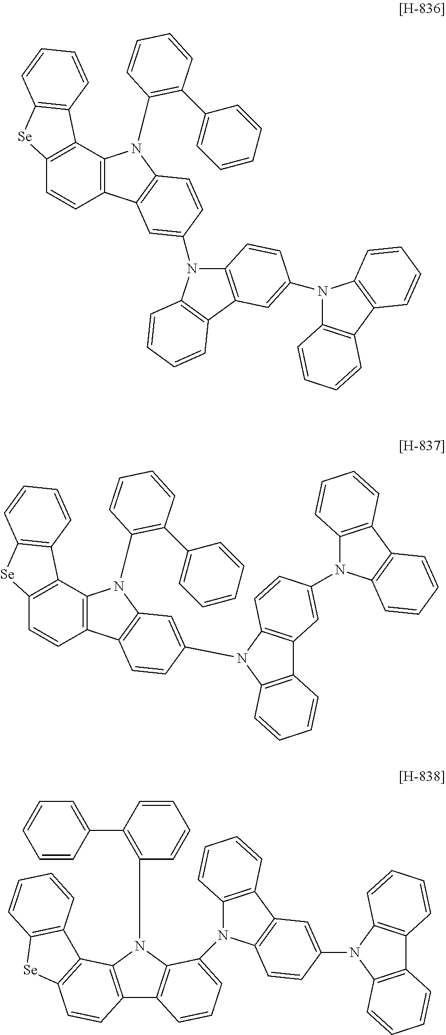 [SMILES: c1ccc(-c2ccccc2-n2c3ccc(-n4c5ccccc5c5cc(-n6c7ccccc7c7ccccc76)ccc54)cc3c3ccc4[se]c5ccccc5c4c32)cc1 ; c1ccc(-c2ccccc2-n2c3cc(-n4c5ccccc5c5cc(-n6c7ccccc7c7ccccc76)ccc54)ccc3c3ccc4[se]c5ccccc5c4c32)cc1 ; c1ccc(-c2ccccc2-n2c3c(-n4c5ccccc5c5cc(-n6c7ccccc7c7ccccc76)ccc54)cccc3c3ccc4[se]c5ccccc5c4c32)cc1]

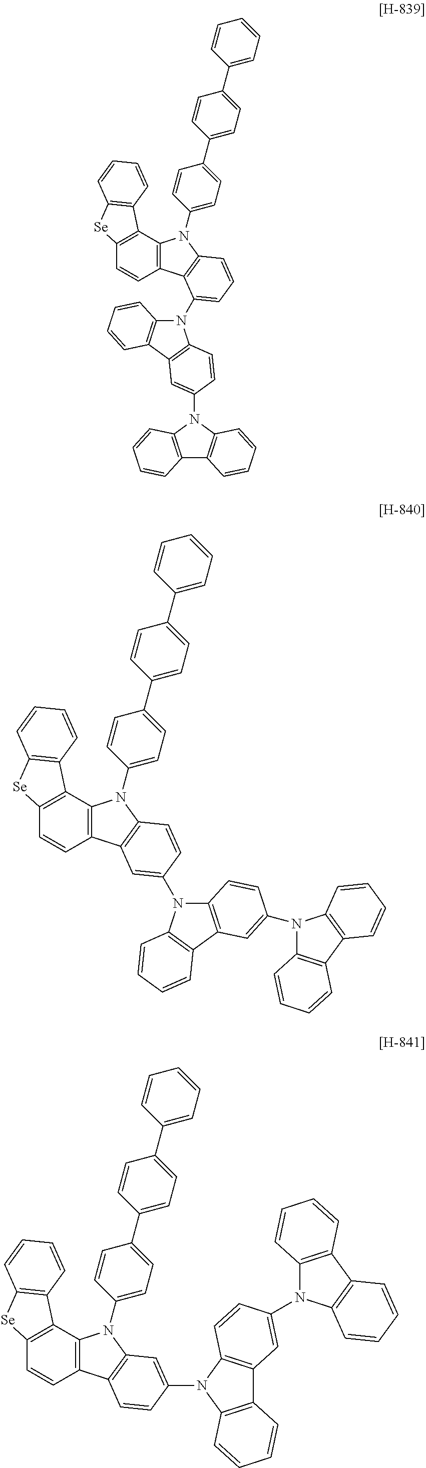 [SMILES: c1ccc(-c2ccc(-c3ccc(-n4c5cccc(-n6c7ccccc7c7cc(-n8c9ccccc9c9ccccc98)ccc76)c5c5ccc6[se]c7ccccc7c6c54)cc3)cc2)cc1 ; c1ccc(-c2ccc(-c3ccc(-n4c5ccc(-n6c7ccccc7c7cc(-n8c9ccccc9c9ccccc98)ccc76)cc5c5ccc6[se]c7ccccc7c6c54)cc3)cc2)cc1 ; c1ccc(-c2ccc(-c3ccc(-n4c5cc(-n6c7ccccc7c7cc(-n8c9ccccc9c9ccccc98)ccc76)ccc5c5ccc6[se]c7ccccc7c6c54)cc3)cc2)cc1]

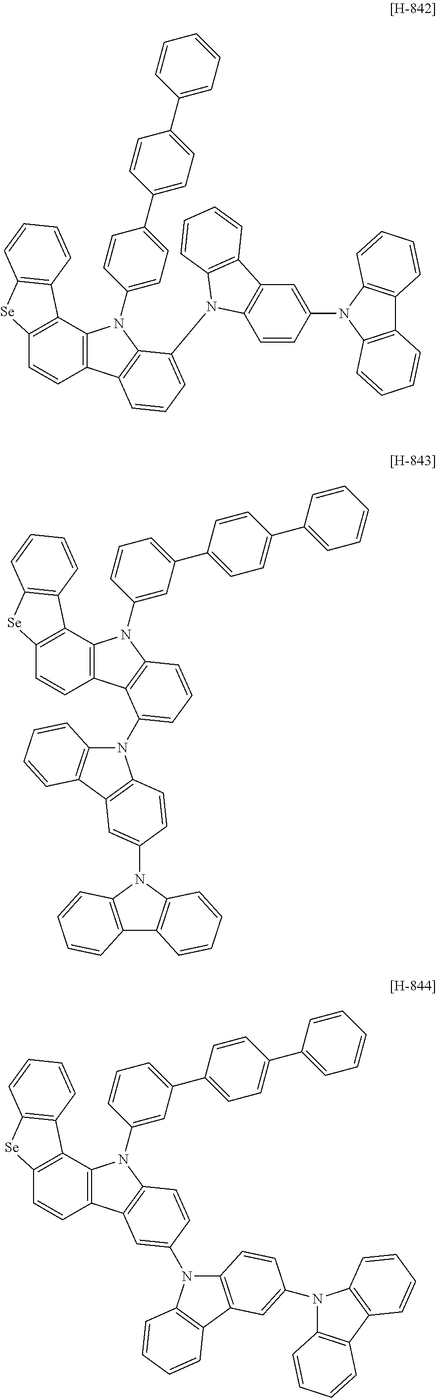 [SMILES: c1ccc(-c2ccc(-c3ccc(-n4c5c(-n6c7ccccc7c7cc(-n8c9ccccc9c9ccccc98)ccc76)cccc5c5ccc6[se]c7ccccc7c6c54)cc3)cc2)cc1 ; c1ccc(-c2ccc(-c3cccc(-n4c5cccc(-n6c7ccccc7c7cc(-n8c9ccccc9c9ccccc98)ccc76)c5c5ccc6[se]c7ccccc7c6c54)c3)cc2)cc1 ; c1ccc(-c2ccc(-c3cccc(-n4c5ccc(-n6c7ccccc7c7cc(-n8c9ccccc9c9ccccc98)ccc76)cc5c5ccc6[se]c7ccccc7c6c54)c3)cc2)cc1]

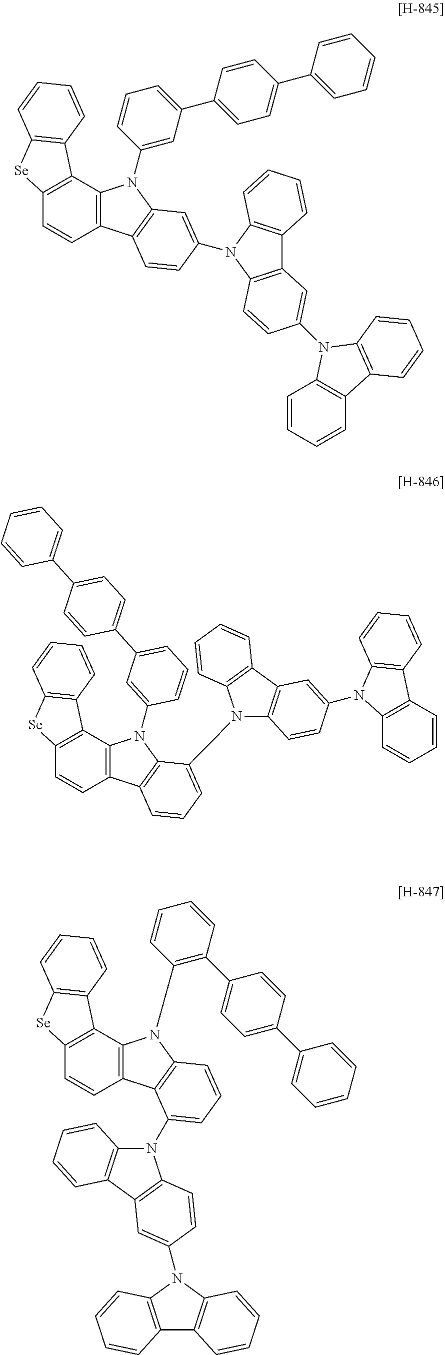 [SMILES: c1ccc(-c2ccc(-c3cccc(-n4c5cc(-n6c7ccccc7c7cc(-n8c9ccccc9c9ccccc98)ccc76)ccc5c5ccc6[se]c7ccccc7c6c54)c3)cc2)cc1 ; c1ccc(-c2ccc(-c3cccc(-n4c5c(-n6c7ccccc7c7cc(-n8c9ccccc9c9ccccc98)ccc76)cccc5c5ccc6[se]c7ccccc7c6c54)c3)cc2)cc1 ; c1ccc(-c2ccc(-c3ccccc3-n3c4cccc(-n5c6ccccc6c6cc(-n7c8ccccc8c8ccccc87)ccc65)c4c4ccc5[se]c6ccccc6c5c43)cc2)cc1]

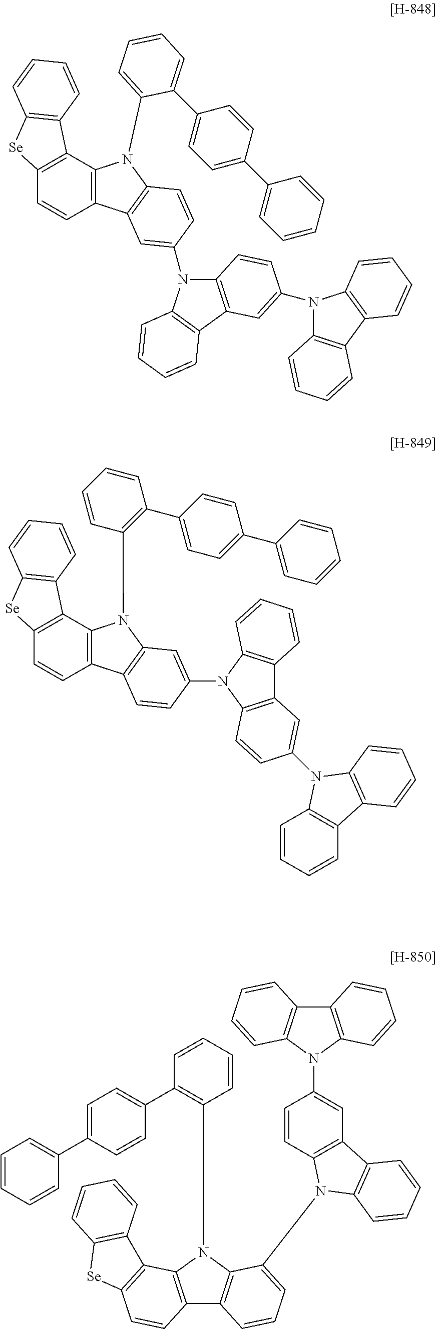 [SMILES: c1ccc(-c2ccc(-c3ccccc3-n3c4ccc(-n5c6ccccc6c6cc(-n7c8ccccc8c8ccccc87)ccc65)cc4c4ccc5[se]c6ccccc6c5c43)cc2)cc1 ; c1ccc(-c2ccc(-c3ccccc3-n3c4cc(-n5c6ccccc6c6cc(-n7c8ccccc8c8ccccc87)ccc65)ccc4c4ccc5[se]c6ccccc6c5c43)cc2)cc1 ; c1ccc(-c2ccc(-c3ccccc3-n3c4c(-n5c6ccccc6c6cc(-n7c8ccccc8c8ccccc87)ccc65)cccc4c4ccc5[se]c6ccccc6c5c43)cc2)cc1]

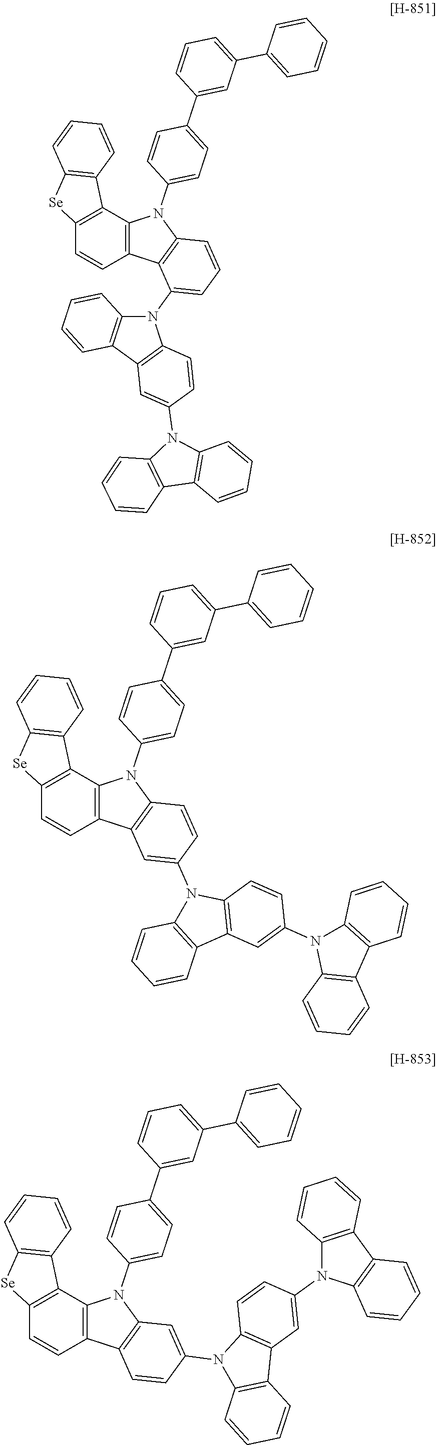 [SMILES: c1ccc(-c2cccc(-c3ccc(-n4c5cccc(-n6c7ccccc7c7cc(-n8c9ccccc9c9ccccc98)ccc76)c5c5ccc6[se]c7ccccc7c6c54)cc3)c2)cc1 ; c1ccc(-c2cccc(-c3ccc(-n4c5ccc(-n6c7ccccc7c7cc(-n8c9ccccc9c9ccccc98)ccc76)cc5c5ccc6[se]c7ccccc7c6c54)cc3)c2)cc1 ; c1ccc(-c2cccc(-c3ccc(-n4c5cc(-n6c7ccccc7c7cc(-n8c9ccccc9c9ccccc98)ccc76)ccc5c5ccc6[se]c7ccccc7c6c54)cc3)c2)cc1]

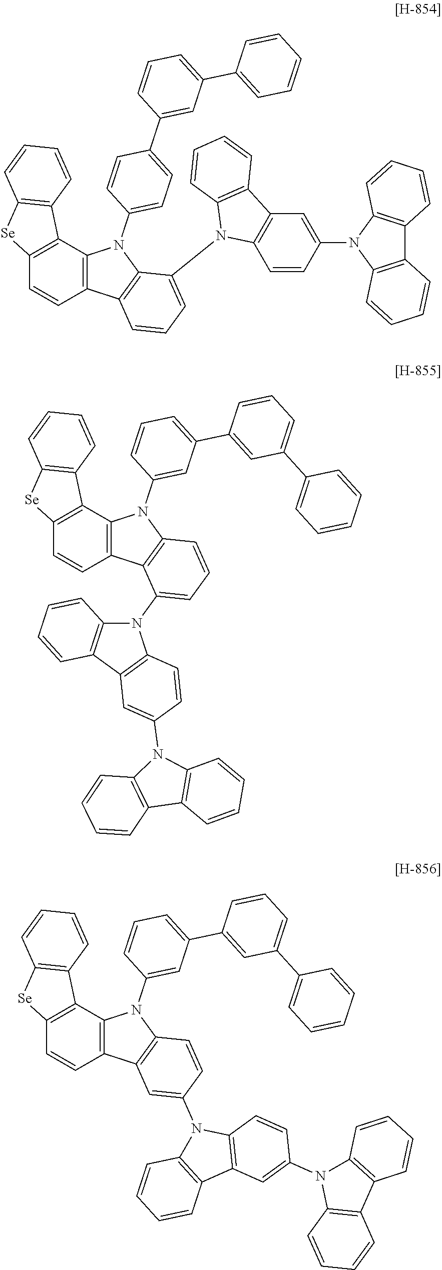 [SMILES: c1ccc(-c2cccc(-c3ccc(-n4c5c(-n6c7ccccc7c7cc(-n8c9ccccc9c9ccccc98)ccc76)cccc5c5ccc6[se]c7ccccc7c6c54)cc3)c2)cc1 ; c1ccc(-c2cccc(-c3cccc(-n4c5cccc(-n6c7ccccc7c7cc(-n8c9ccccc9c9ccccc98)ccc76)c5c5ccc6[se]c7ccccc7c6c54)c3)c2)cc1 ; c1ccc(-c2cccc(-c3cccc(-n4c5ccc(-n6c7ccccc7c7cc(-n8c9ccccc9c9ccccc98)ccc76)cc5c5ccc6[se]c7ccccc7c6c54)c3)c2)cc1]

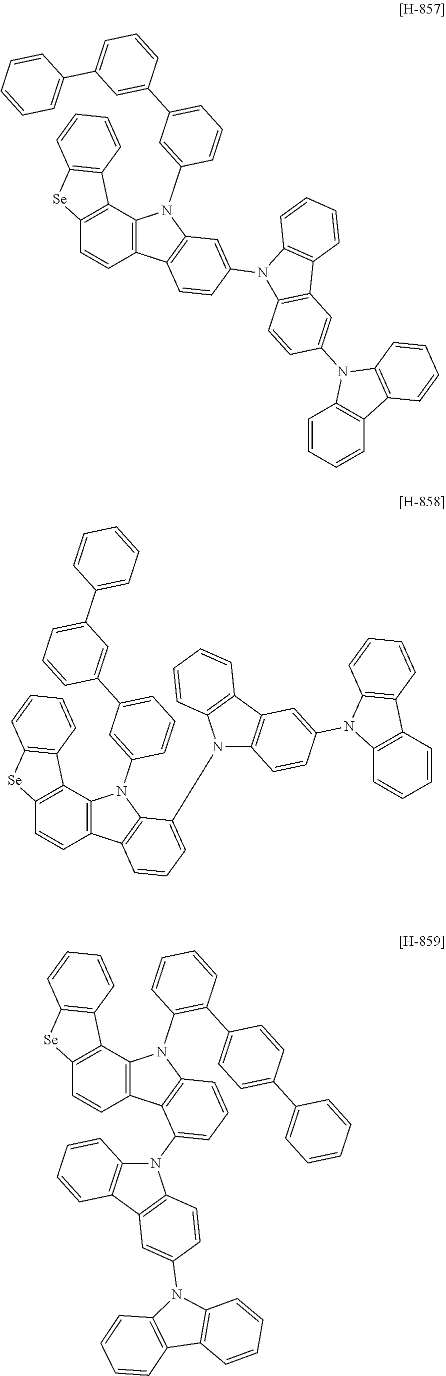 [SMILES: c1ccc(-c2cccc(-c3cccc(-n4c5cc(-n6c7ccccc7c7cc(-n8c9ccccc9c9ccccc98)ccc76)ccc5c5ccc6[se]c7ccccc7c6c54)c3)c2)cc1 ; c1ccc(-c2cccc(-c3cccc(-n4c5c(-n6c7ccccc7c7cc(-n8c9ccccc9c9ccccc98)ccc76)cccc5c5ccc6[se]c7ccccc7c6c54)c3)c2)cc1 ; c1ccc(-c2ccc(-c3ccccc3-n3c4cccc(-n5c6ccccc6c6cc(-n7c8ccccc8c8ccccc87)ccc65)c4c4ccc5[se]c6ccccc6c5c43)cc2)cc1]

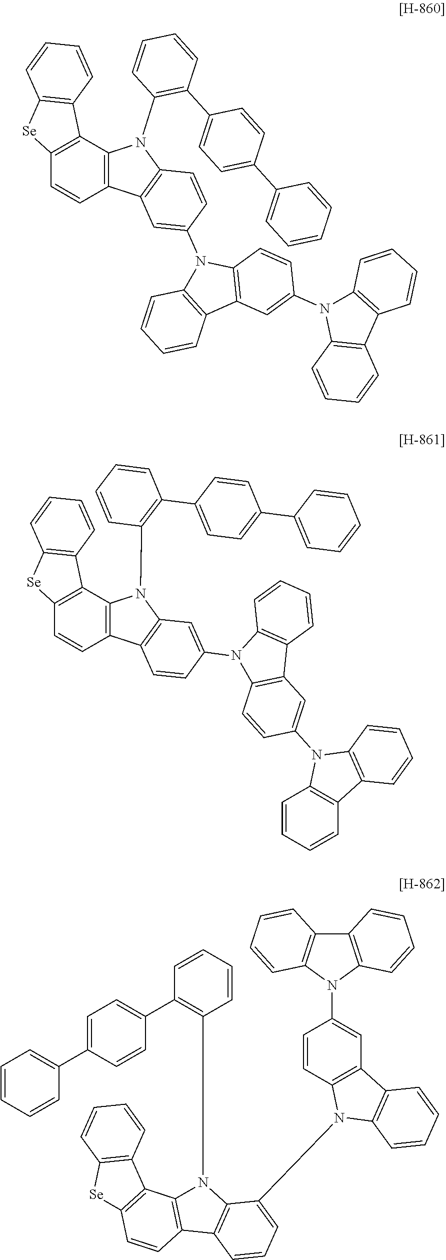 [SMILES: c1ccc(-c2ccc(-c3ccccc3-n3c4ccc(-n5c6ccccc6c6cc(-n7c8ccccc8c8ccccc87)ccc65)cc4c4ccc5[se]c6ccccc6c5c43)cc2)cc1 ; c1ccc(-c2ccc(-c3ccccc3-n3c4cc(-n5c6ccccc6c6cc(-n7c8ccccc8c8ccccc87)ccc65)ccc4c4ccc5[se]c6ccccc6c5c43)cc2)cc1 ; c1ccc(-c2ccc(-c3ccccc3-n3c4c(-n5c6ccccc6c6cc(-n7c8ccccc8c8ccccc87)ccc65)cccc4c4ccc5[se]c6ccccc6c5c43)cc2)cc1]

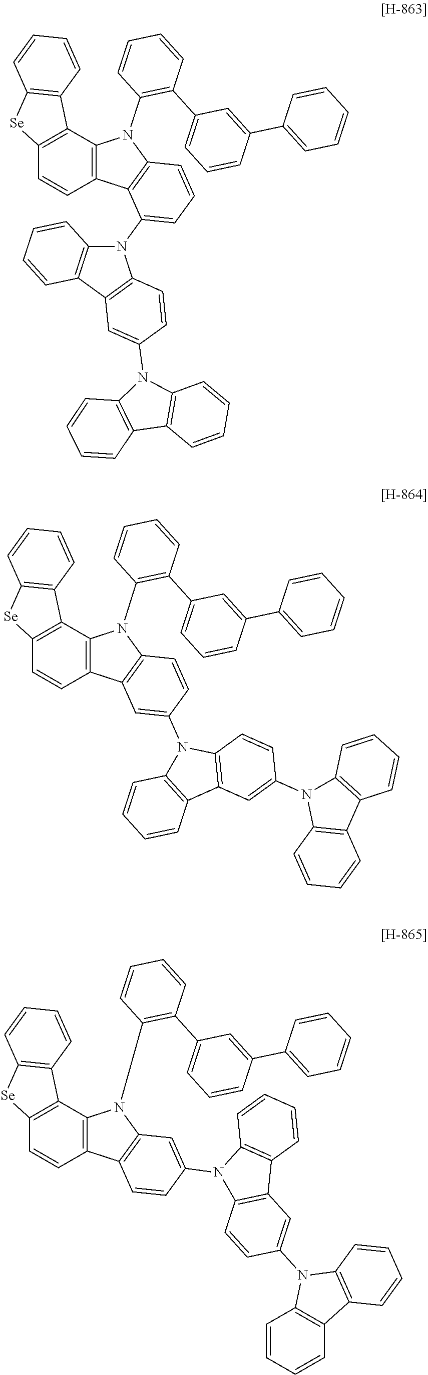 [SMILES: c1ccc(-c2cccc(-c3ccccc3-n3c4cccc(-n5c6ccccc6c6cc(-n7c8ccccc8c8ccccc87)ccc65)c4c4ccc5[se]c6ccccc6c5c43)c2)cc1 ; c1ccc(-c2cccc(-c3ccccc3-n3c4ccc(-n5c6ccccc6c6cc(-n7c8ccccc8c8ccccc87)ccc65)cc4c4ccc5[se]c6ccccc6c5c43)c2)cc1 ; c1ccc(-c2cccc(-c3ccccc3-n3c4cc(-n5c6ccccc6c6cc(-n7c8ccccc8c8ccccc87)ccc65)ccc4c4ccc5[se]c6ccccc6c5c43)c2)cc1]

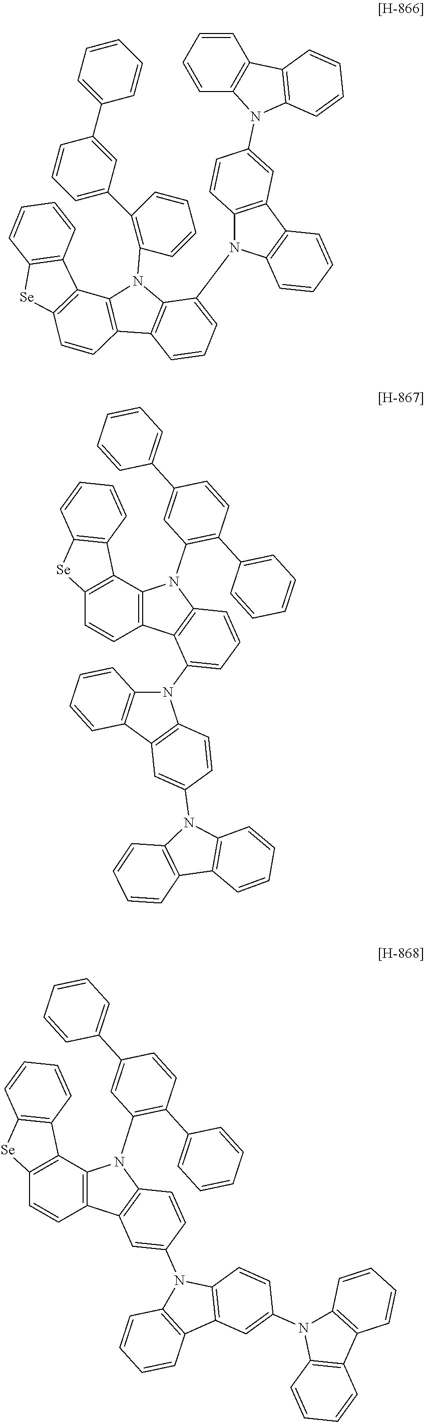 [SMILES: c1ccc(-c2cccc(-c3ccccc3-n3c4c(-n5c6ccccc6c6cc(-n7c8ccccc8c8ccccc87)ccc65)cccc4c4ccc5[se]c6ccccc6c5c43)c2)cc1 ; c1ccc(-c2ccc(-c3ccccc3)c(-n3c4cccc(-n5c6ccccc6c6cc(-n7c8ccccc8c8ccccc87)ccc65)c4c4ccc5[se]c6ccccc6c5c43)c2)cc1 ; c1ccc(-c2ccc(-c3ccccc3)c(-n3c4ccc(-n5c6ccccc6c6cc(-n7c8ccccc8c8ccccc87)ccc65)cc4c4ccc5[se]c6ccccc6c5c43)c2)cc1]

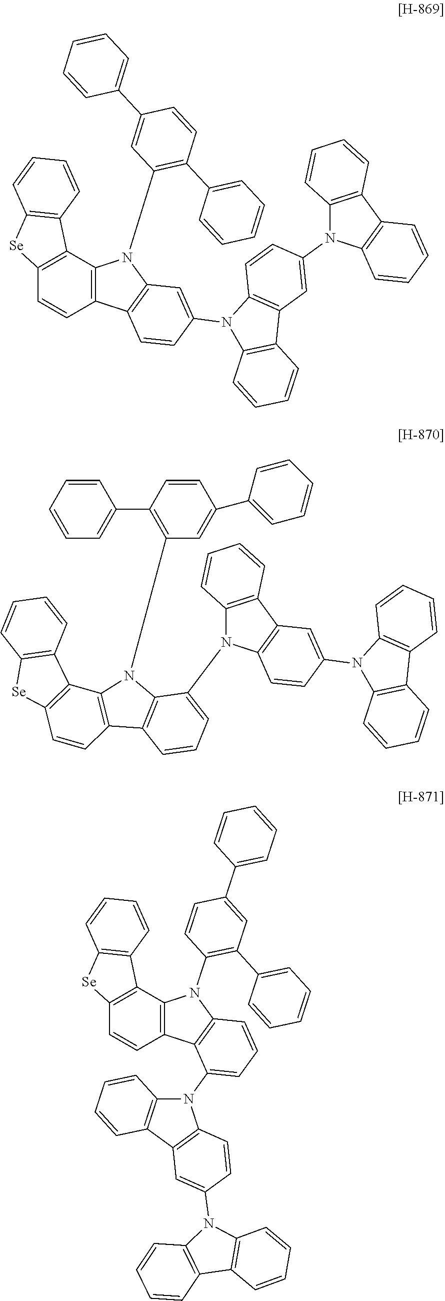 [SMILES: c1ccc(-c2ccc(-c3ccccc3)c(-n3c4cc(-n5c6ccccc6c6cc(-n7c8ccccc8c8ccccc87)ccc65)ccc4c4ccc5[se]c6ccccc6c5c43)c2)cc1 ; c1ccc(-c2ccc(-c3ccccc3)c(-n3c4c(-n5c6ccccc6c6cc(-n7c8ccccc8c8ccccc87)ccc65)cccc4c4ccc5[se]c6ccccc6c5c43)c2)cc1 ; c1ccc(-c2ccc(-n3c4cccc(-n5c6ccccc6c6cc(-n7c8ccccc8c8ccccc87)ccc65)c4c4ccc5[se]c6ccccc6c5c43)c(-c3ccccc3)c2)cc1]

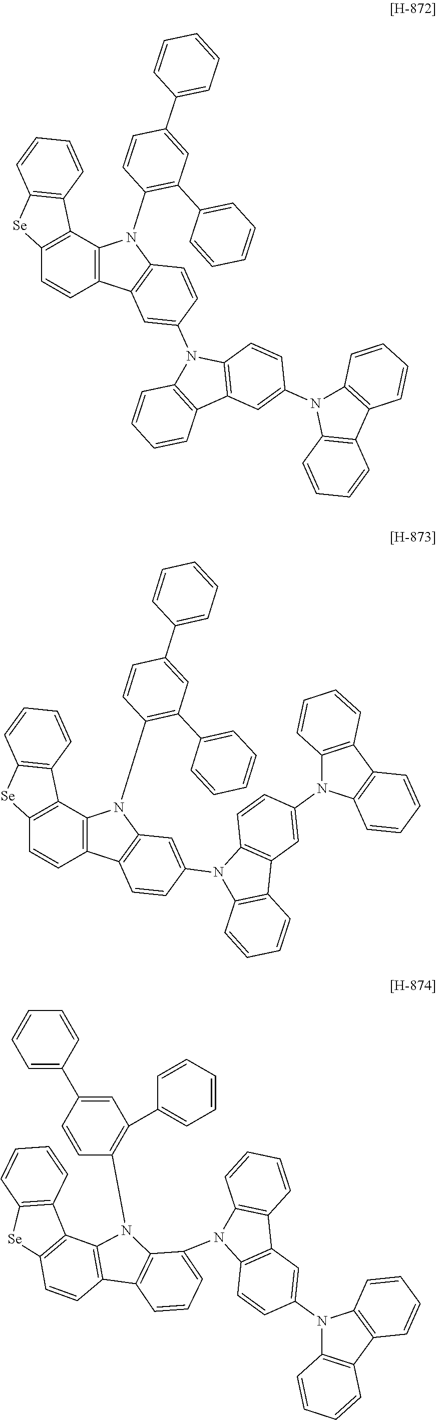 [SMILES: c1ccc(-c2ccc(-n3c4ccc(-n5c6ccccc6c6cc(-n7c8ccccc8c8ccccc87)ccc65)cc4c4ccc5[se]c6ccccc6c5c43)c(-c3ccccc3)c2)cc1 ; c1ccc(-c2ccc(-n3c4cc(-n5c6ccccc6c6cc(-n7c8ccccc8c8ccccc87)ccc65)ccc4c4ccc5[se]c6ccccc6c5c43)c(-c3ccccc3)c2)cc1 ; c1ccc(-c2ccc(-n3c4c(-n5c6ccccc6c6cc(-n7c8ccccc8c8ccccc87)ccc65)cccc4c4ccc5[se]c6ccccc6c5c43)c(-c3ccccc3)c2)cc1]

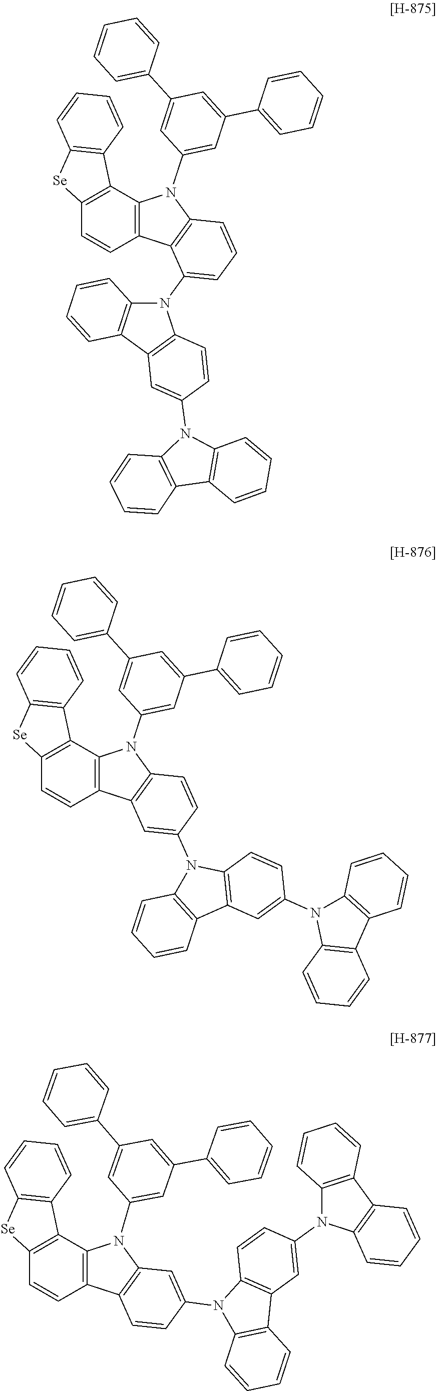 [SMILES: c1ccc(-c2cc(-c3ccccc3)cc(-n3c4cccc(-n5c6ccccc6c6cc(-n7c8ccccc8c8ccccc87)ccc65)c4c4ccc5[se]c6ccccc6c5c43)c2)cc1 ; c1ccc(-c2cc(-c3ccccc3)cc(-n3c4ccc(-n5c6ccccc6c6cc(-n7c8ccccc8c8ccccc87)ccc65)cc4c4ccc5[se]c6ccccc6c5c43)c2)cc1 ; c1ccc(-c2cc(-c3ccccc3)cc(-n3c4cc(-n5c6ccccc6c6cc(-n7c8ccccc8c8ccccc87)ccc65)ccc4c4ccc5[se]c6ccccc6c5c43)c2)cc1]

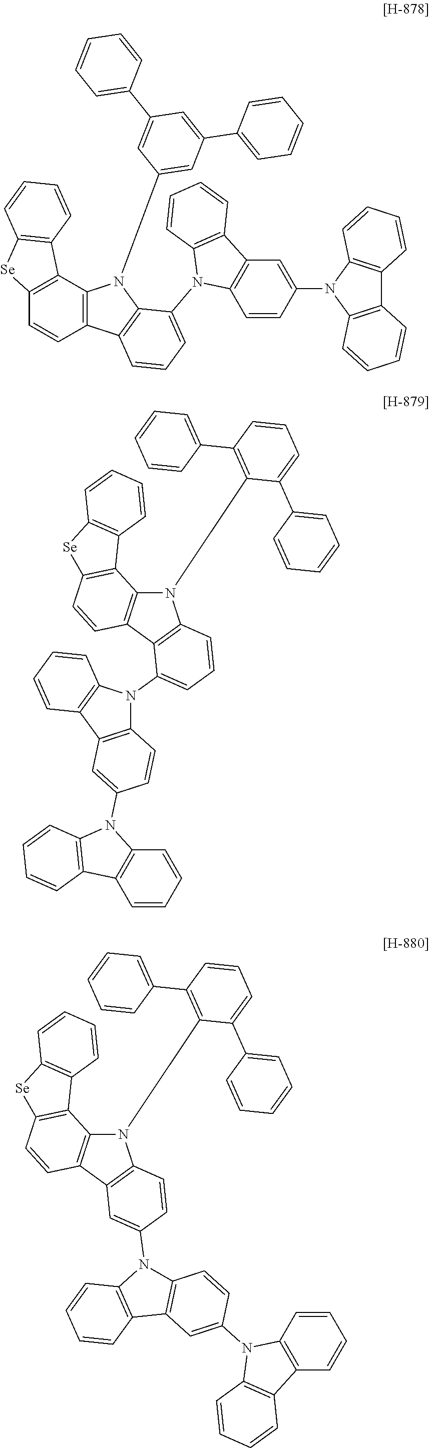

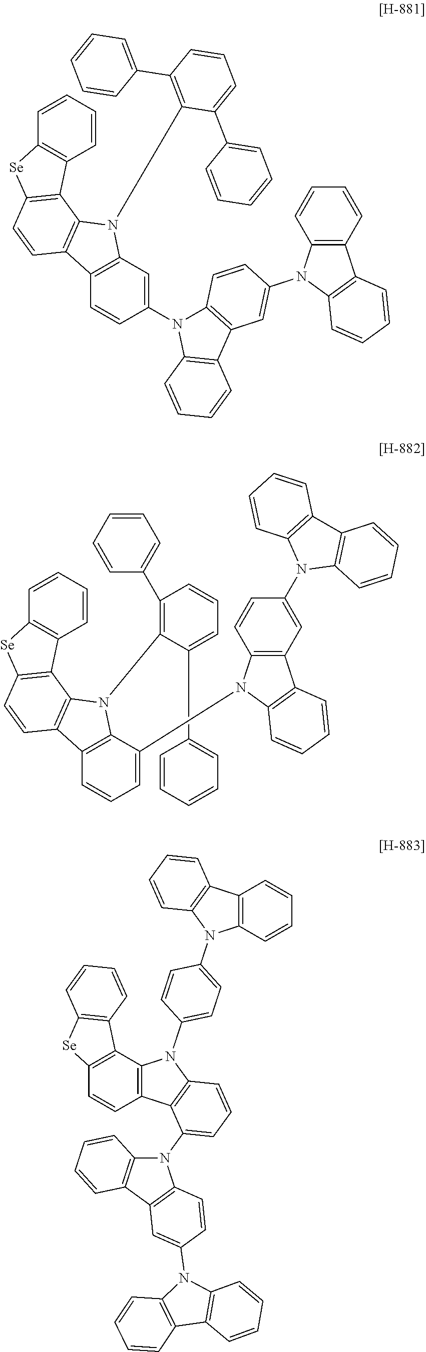 [SMILES: c1ccc(-c2cccc(-c3ccccc3)c2-n2c3cc(-n4c5ccccc5c5cc(-n6c7ccccc7c7ccccc76)ccc54)ccc3c3ccc4[se]c5ccccc5c4c32)cc1 ; c1ccc(-c2cccc(-c3ccccc3)c2-n2c3c(-n4c5ccccc5c5cc(-n6c7ccccc7c7ccccc76)ccc54)cccc3c3ccc4[se]c5ccccc5c4c32)cc1 ; c1ccc2c(c1)[se]c1ccc3c4c(-n5c6ccccc6c6cc(-n7c8ccccc8c8ccccc87)ccc65)cccc4n(-c4ccc(-n5c6ccccc6c6ccccc65)cc4)c3c12]

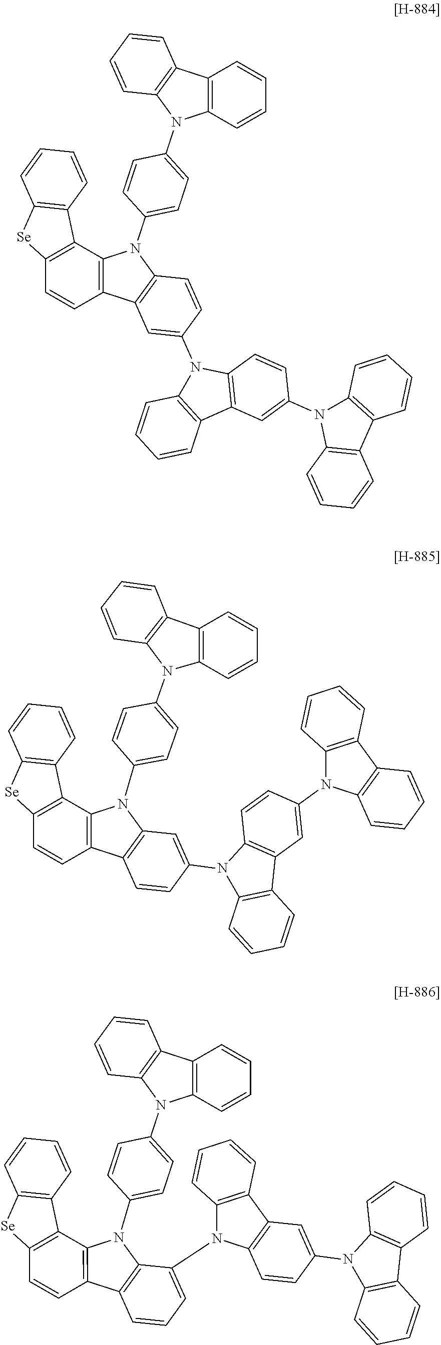 [SMILES: c1ccc2c(c1)[se]c1ccc3c4cc(-n5c6ccccc6c6cc(-n7c8ccccc8c8ccccc87)ccc65)ccc4n(-c4ccc(-n5c6ccccc6c6ccccc65)cc4)c3c12 ; c1ccc2c(c1)[se]c1ccc3c4ccc(-n5c6ccccc6c6cc(-n7c8ccccc8c8ccccc87)ccc65)cc4n(-c4ccc(-n5c6ccccc6c6ccccc65)cc4)c3c12 ; c1ccc2c(c1)[se]c1ccc3c4cccc(-n5c6ccccc6c6cc(-n7c8ccccc8c8ccccc87)ccc65)c4n(-c4ccc(-n5c6ccccc6c6ccccc65)cc4)c3c12]

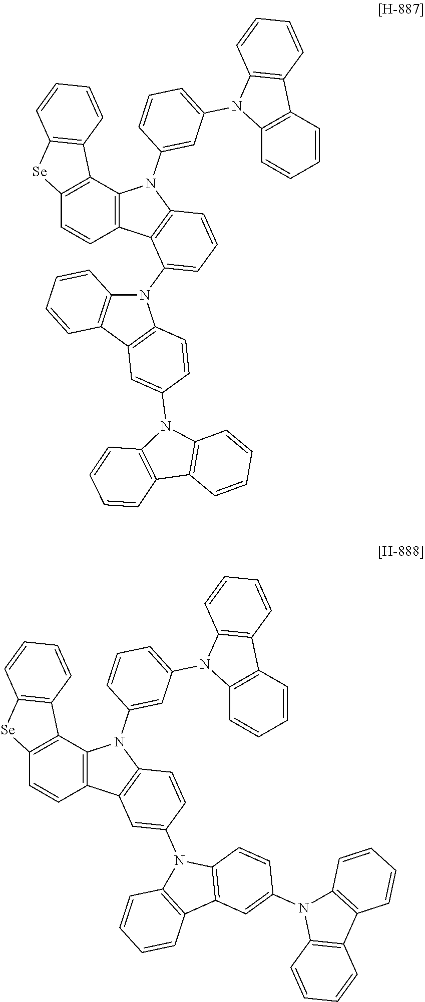 [SMILES: c1cc(-n2c3ccccc3c3ccccc32)cc(-n2c3cccc(-n4c5ccccc5c5cc(-n6c7ccccc7c7ccccc76)ccc54)c3c3ccc4[se]c5ccccc5c4c32)c1 ; c1cc(-n2c3ccccc3c3ccccc32)cc(-n2c3ccc(-n4c5ccccc5c5cc(-n6c7ccccc7c7ccccc76)ccc54)cc3c3ccc4[se]c5ccccc5c4c32)c1]

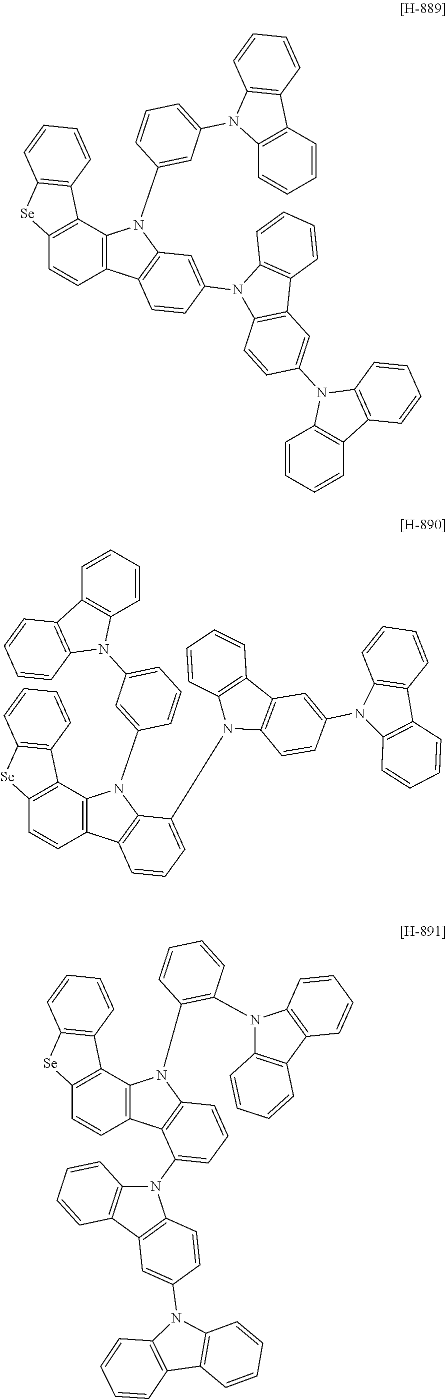 [SMILES: c1cc(-n2c3ccccc3c3ccccc32)cc(-n2c3cc(-n4c5ccccc5c5cc(-n6c7ccccc7c7ccccc76)ccc54)ccc3c3ccc4[se]c5ccccc5c4c32)c1 ; c1cc(-n2c3ccccc3c3ccccc32)cc(-n2c3c(-n4c5ccccc5c5cc(-n6c7ccccc7c7ccccc76)ccc54)cccc3c3ccc4[se]c5ccccc5c4c32)c1 ; c1ccc(-n2c3cccc(-n4c5ccccc5c5cc(-n6c7ccccc7c7ccccc76)ccc54)c3c3ccc4[se]c5ccccc5c4c32)c(-n2c3ccccc3c3ccccc32)c1]

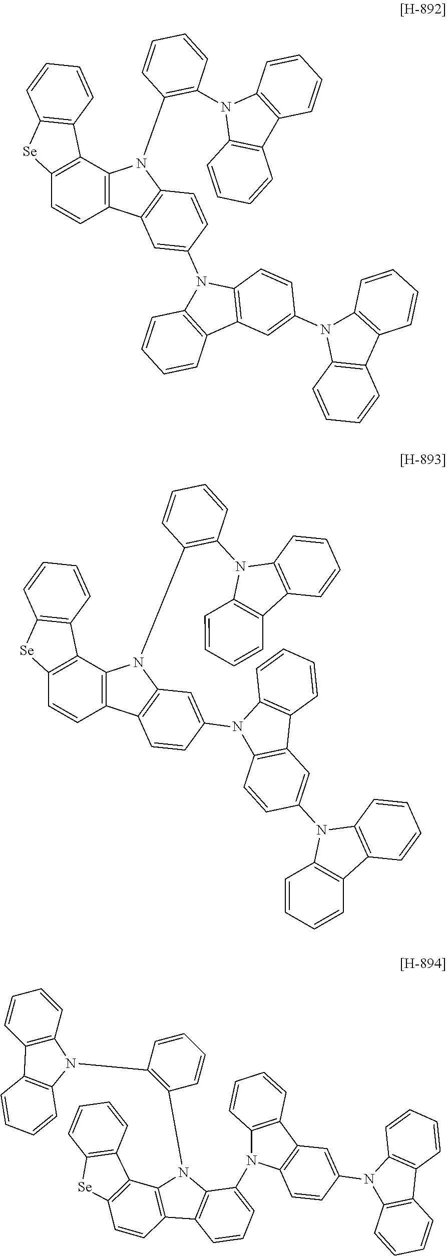 [SMILES: c1ccc(-n2c3ccc(-n4c5ccccc5c5cc(-n6c7ccccc7c7ccccc76)ccc54)cc3c3ccc4[se]c5ccccc5c4c32)c(-n2c3ccccc3c3ccccc32)c1 ; c1ccc(-n2c3cc(-n4c5ccccc5c5cc(-n6c7ccccc7c7ccccc76)ccc54)ccc3c3ccc4[se]c5ccccc5c4c32)c(-n2c3ccccc3c3ccccc32)c1 ; c1ccc(-n2c3c(-n4c5ccccc5c5cc(-n6c7ccccc7c7ccccc76)ccc54)cccc3c3ccc4[se]c5ccccc5c4c32)c(-n2c3ccccc3c3ccccc32)c1]

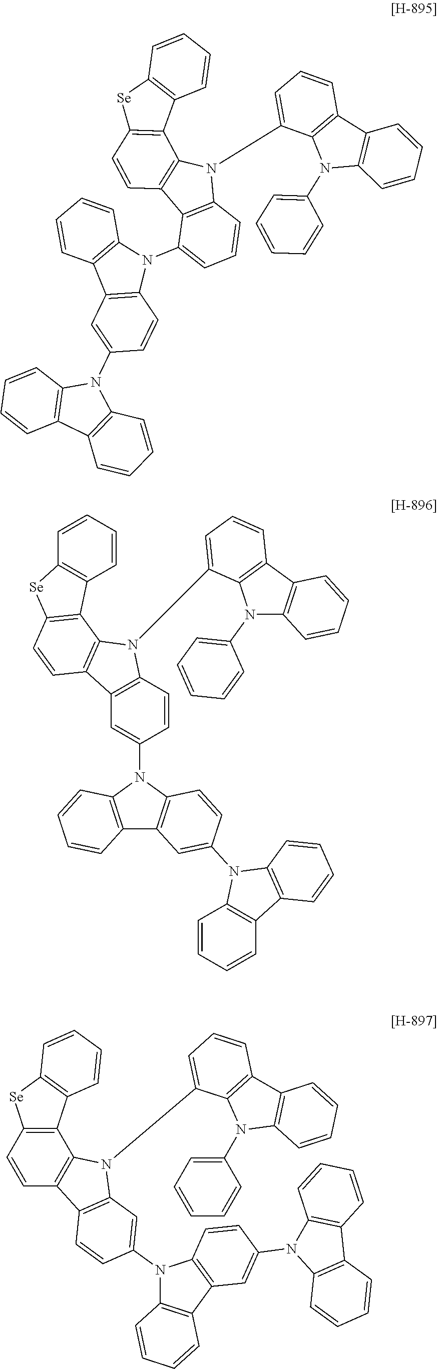 [SMILES: c1ccc(-n2c3ccccc3c3cccc(-n4c5cccc(-n6c7ccccc7c7cc(-n8c9ccccc9c9ccccc98)ccc76)c5c5ccc6[se]c7ccccc7c6c54)c32)cc1 ; c1ccc(-n2c3ccccc3c3cccc(-n4c5ccc(-n6c7ccccc7c7cc(-n8c9ccccc9c9ccccc98)ccc76)cc5c5ccc6[se]c7ccccc7c6c54)c32)cc1 ; c1ccc(-n2c3ccccc3c3cccc(-n4c5cc(-n6c7ccccc7c7cc(-n8c9ccccc9c9ccccc98)ccc76)ccc5c5ccc6[se]c7ccccc7c6c54)c32)cc1]

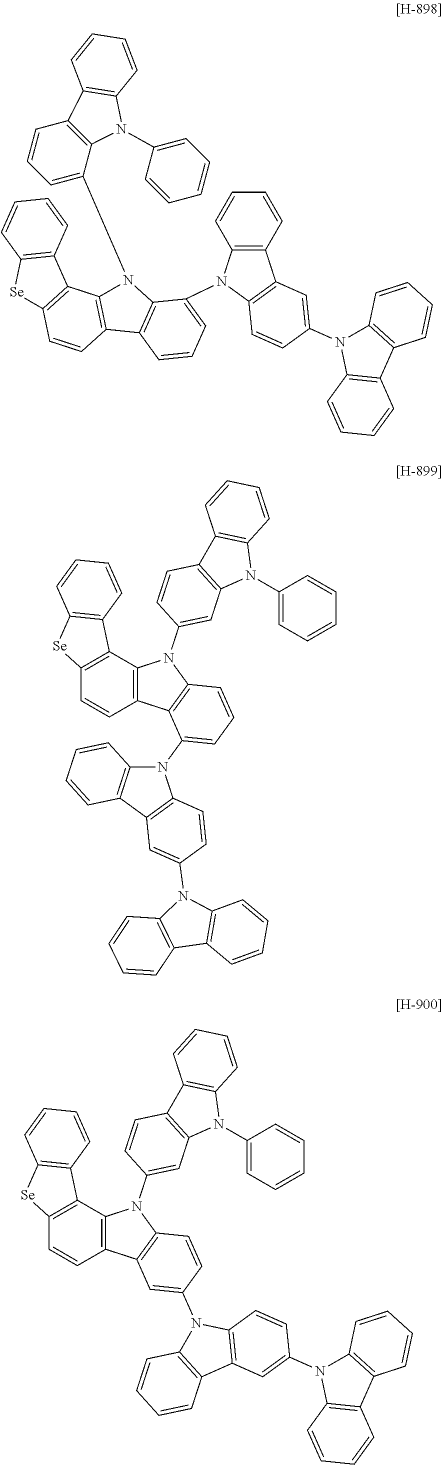 [SMILES: c1ccc(-n2c3ccccc3c3cccc(-n4c5c(-n6c7ccccc7c7cc(-n8c9ccccc9c9ccccc98)ccc76)cccc5c5ccc6[se]c7ccccc7c6c54)c32)cc1 ; c1ccc(-n2c3ccccc3c3ccc(-n4c5cccc(-n6c7ccccc7c7cc(-n8c9ccccc9c9ccccc98)ccc76)c5c5ccc6[se]c7ccccc7c6c54)cc32)cc1 ; c1ccc(-n2c3ccccc3c3ccc(-n4c5ccc(-n6c7ccccc7c7cc(-n8c9ccccc9c9ccccc98)ccc76)cc5c5ccc6[se]c7ccccc7c6c54)cc32)cc1]

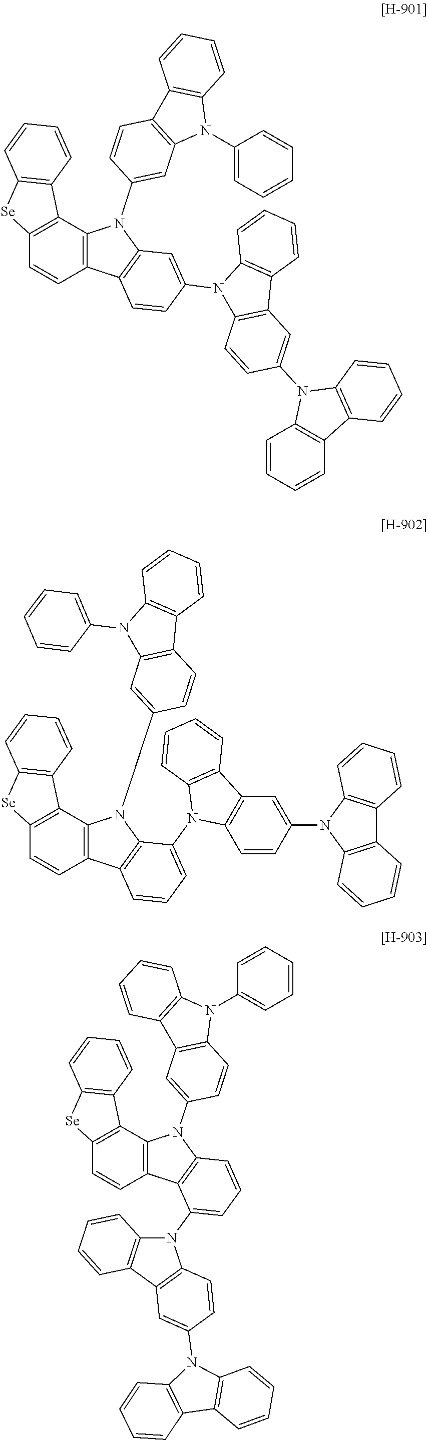 [SMILES: c1ccc(-n2c3ccccc3c3ccc(-n4c5cc(-n6c7ccccc7c7cc(-n8c9ccccc9c9ccccc98)ccc76)ccc5c5ccc6[se]c7ccccc7c6c54)cc32)cc1 ; c1ccc(-n2c3ccccc3c3ccc(-n4c5c(-n6c7ccccc7c7cc(-n8c9ccccc9c9ccccc98)ccc76)cccc5c5ccc6[se]c7ccccc7c6c54)cc32)cc1 ; c1ccc(-n2c3ccccc3c3cc(-n4c5cccc(-n6c7ccccc7c7cc(-n8c9ccccc9c9ccccc98)ccc76)c5c5ccc6[se]c7ccccc7c6c54)ccc32)cc1]

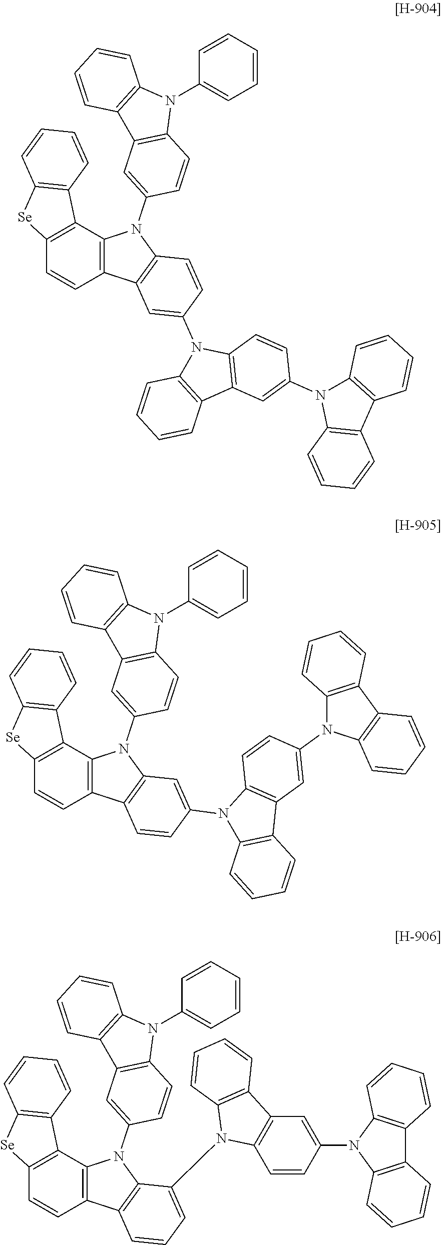 [SMILES: c1ccc(-n2c3ccccc3c3cc(-n4c5ccc(-n6c7ccccc7c7cc(-n8c9ccccc9c9ccccc98)ccc76)cc5c5ccc6[se]c7ccccc7c6c54)ccc32)cc1 ; c1ccc(-n2c3ccccc3c3cc(-n4c5cc(-n6c7ccccc7c7cc(-n8c9ccccc9c9ccccc98)ccc76)ccc5c5ccc6[se]c7ccccc7c6c54)ccc32)cc1 ; c1ccc(-n2c3ccccc3c3cc(-n4c5c(-n6c7ccccc7c7cc(-n8c9ccccc9c9ccccc98)ccc76)cccc5c5ccc6[se]c7ccccc7c6c54)ccc32)cc1]

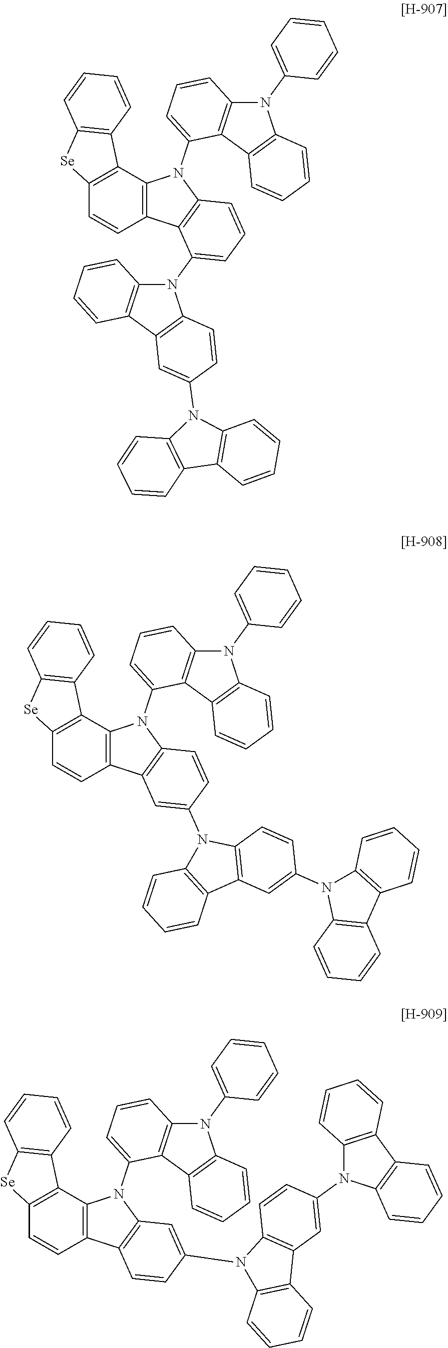 [SMILES: c1ccc(-n2c3ccccc3c3c(-n4c5cccc(-n6c7ccccc7c7cc(-n8c9ccccc9c9ccccc98)ccc76)c5c5ccc6[se]c7ccccc7c6c54)cccc32)cc1 ; c1ccc(-n2c3ccccc3c3c(-n4c5ccc(-n6c7ccccc7c7cc(-n8c9ccccc9c9ccccc98)ccc76)cc5c5ccc6[se]c7ccccc7c6c54)cccc32)cc1 ; c1ccc(-n2c3ccccc3c3c(-n4c5cc(-n6c7ccccc7c7cc(-n8c9ccccc9c9ccccc98)ccc76)ccc5c5ccc6[se]c7ccccc7c6c54)cccc32)cc1]

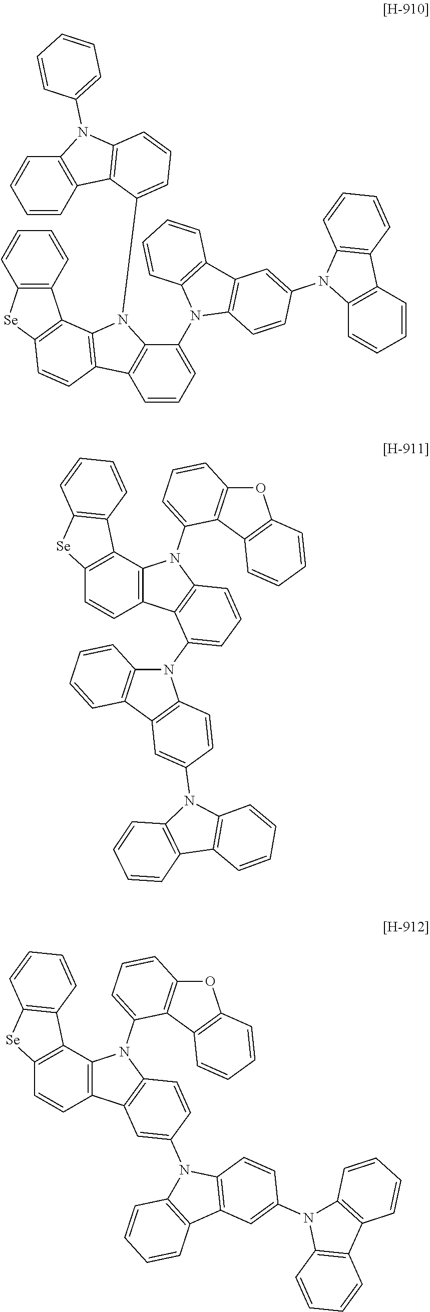 [SMILES: c1ccc(-n2c3ccccc3c3c(-n4c5c(-n6c7ccccc7c7cc(-n8c9ccccc9c9ccccc98)ccc76)cccc5c5ccc6[se]c7ccccc7c6c54)cccc32)cc1 ; c1ccc2c(c1)oc1cccc(-n3c4cccc(-n5c6ccccc6c6cc(-n7c8ccccc8c8ccccc87)ccc65)c4c4ccc5[se]c6ccccc6c5c43)c12 ; c1ccc2c(c1)oc1cccc(-n3c4ccc(-n5c6ccccc6c6cc(-n7c8ccccc8c8ccccc87)ccc65)cc4c4ccc5[se]c6ccccc6c5c43)c12]

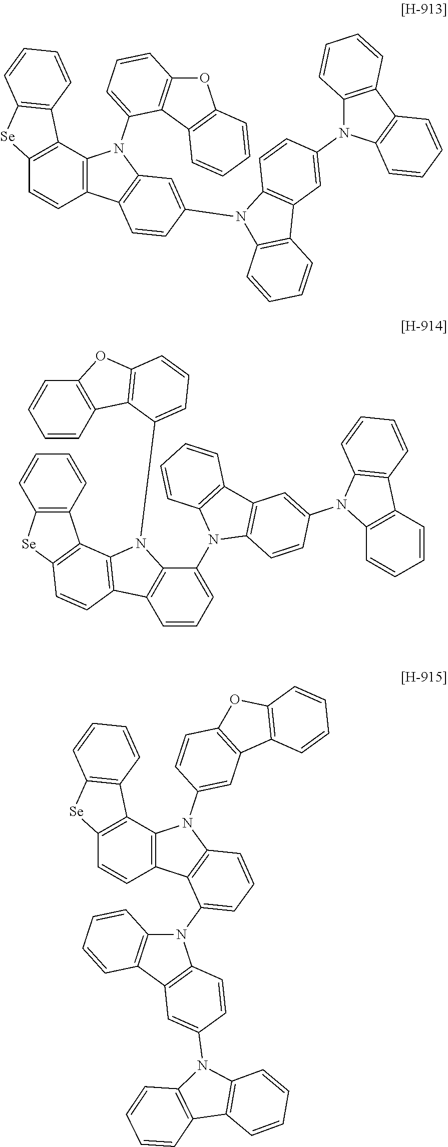 [SMILES: c1ccc2c(c1)oc1cccc(-n3c4cc(-n5c6ccccc6c6cc(-n7c8ccccc8c8ccccc87)ccc65)ccc4c4ccc5[se]c6ccccc6c5c43)c12 ; c1ccc2c(c1)oc1cccc(-n3c4c(-n5c6ccccc6c6cc(-n7c8ccccc8c8ccccc87)ccc65)cccc4c4ccc5[se]c6ccccc6c5c43)c12 ; c1ccc2c(c1)oc1ccc(-n3c4cccc(-n5c6ccccc6c6cc(-n7c8ccccc8c8ccccc87)ccc65)c4c4ccc5[se]c6ccccc6c5c43)cc12]

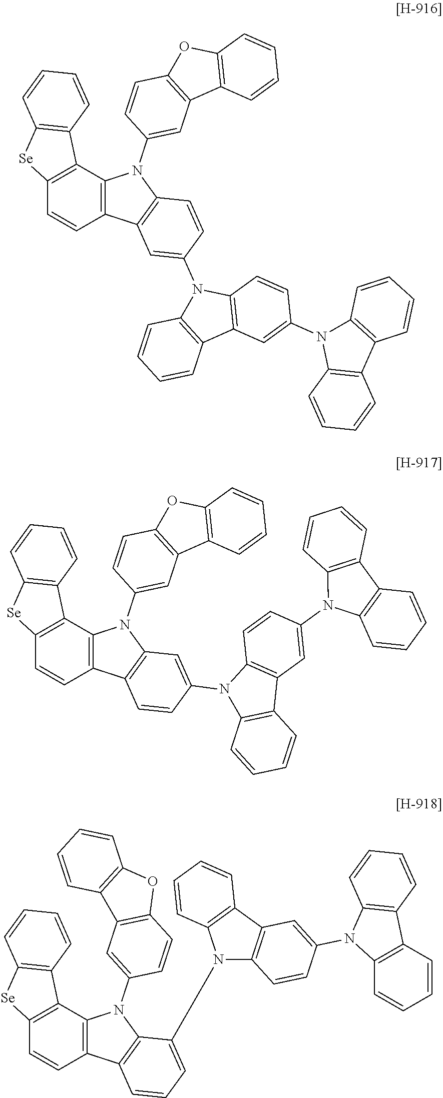 [SMILES: c1ccc2c(c1)oc1ccc(-n3c4ccc(-n5c6ccccc6c6cc(-n7c8ccccc8c8ccccc87)ccc65)cc4c4ccc5[se]c6ccccc6c5c43)cc12 ; c1ccc2c(c1)oc1ccc(-n3c4cc(-n5c6ccccc6c6cc(-n7c8ccccc8c8ccccc87)ccc65)ccc4c4ccc5[se]c6ccccc6c5c43)cc12 ; c1ccc2c(c1)oc1ccc(-n3c4c(-n5c6ccccc6c6cc(-n7c8ccccc8c8ccccc87)ccc65)cccc4c4ccc5[se]c6ccccc6c5c43)cc12]

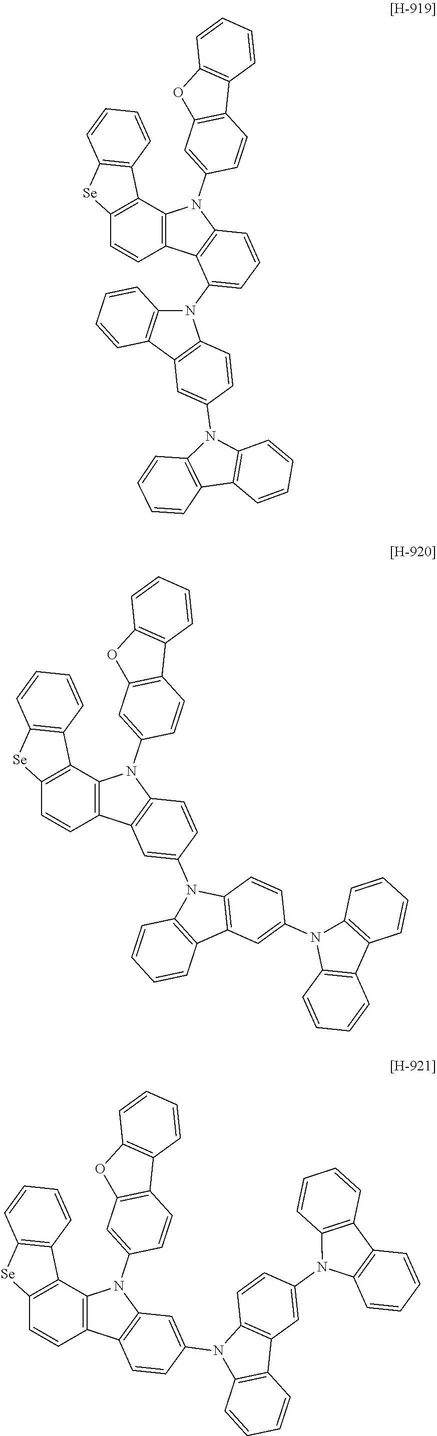 [SMILES: c1ccc2c(c1)oc1cc(-n3c4cccc(-n5c6ccccc6c6cc(-n7c8ccccc8c8ccccc87)ccc65)c4c4ccc5[se]c6ccccc6c5c43)ccc12 ; c1ccc2c(c1)oc1cc(-n3c4ccc(-n5c6ccccc6c6cc(-n7c8ccccc8c8ccccc87)ccc65)cc4c4ccc5[se]c6ccccc6c5c43)ccc12 ; c1ccc2c(c1)oc1cc(-n3c4cc(-n5c6ccccc6c6cc(-n7c8ccccc8c8ccccc87)ccc65)ccc4c4ccc5[se]c6ccccc6c5c43)ccc12]

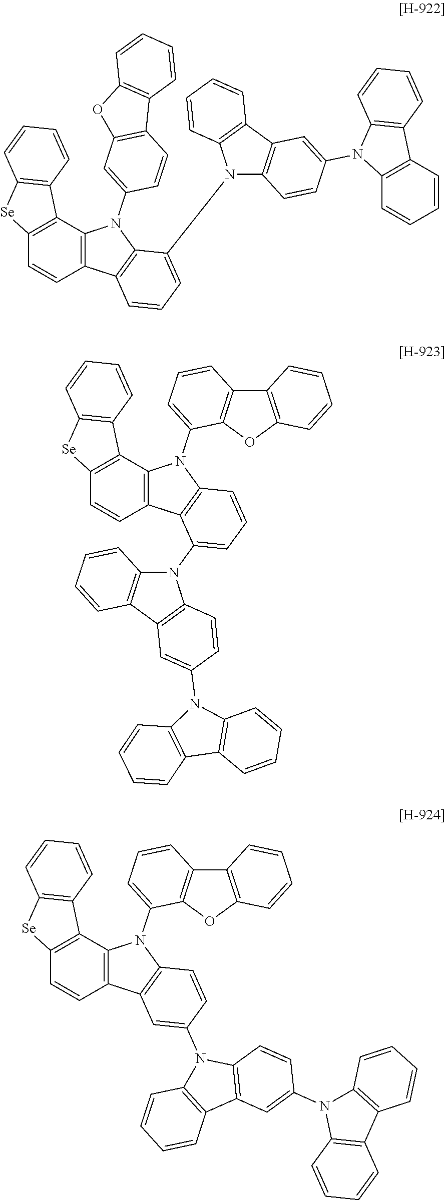 [SMILES: c1ccc2c(c1)oc1cc(-n3c4c(-n5c6ccccc6c6cc(-n7c8ccccc8c8ccccc87)ccc65)cccc4c4ccc5[se]c6ccccc6c5c43)ccc12 ; c1ccc2c(c1)oc1c(-n3c4cccc(-n5c6ccccc6c6cc(-n7c8ccccc8c8ccccc87)ccc65)c4c4ccc5[se]c6ccccc6c5c43)cccc12 ; c1ccc2c(c1)oc1c(-n3c4ccc(-n5c6ccccc6c6cc(-n7c8ccccc8c8ccccc87)ccc65)cc4c4ccc5[se]c6ccccc6c5c43)cccc12]

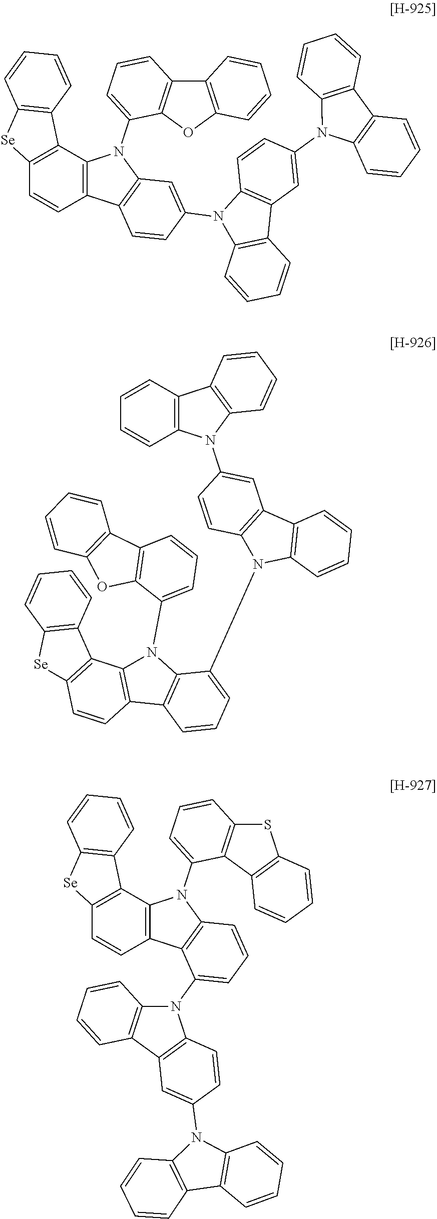 [SMILES: c1ccc2c(c1)oc1c(-n3c4cc(-n5c6ccccc6c6cc(-n7c8ccccc8c8ccccc87)ccc65)ccc4c4ccc5[se]c6ccccc6c5c43)cccc12 ; c1ccc2c(c1)oc1c(-n3c4c(-n5c6ccccc6c6cc(-n7c8ccccc8c8ccccc87)ccc65)cccc4c4ccc5[se]c6ccccc6c5c43)cccc12 ; c1ccc2c(c1)sc1cccc(-n3c4cccc(-n5c6ccccc6c6cc(-n7c8ccccc8c8ccccc87)ccc65)c4c4ccc5[se]c6ccccc6c5c43)c12]

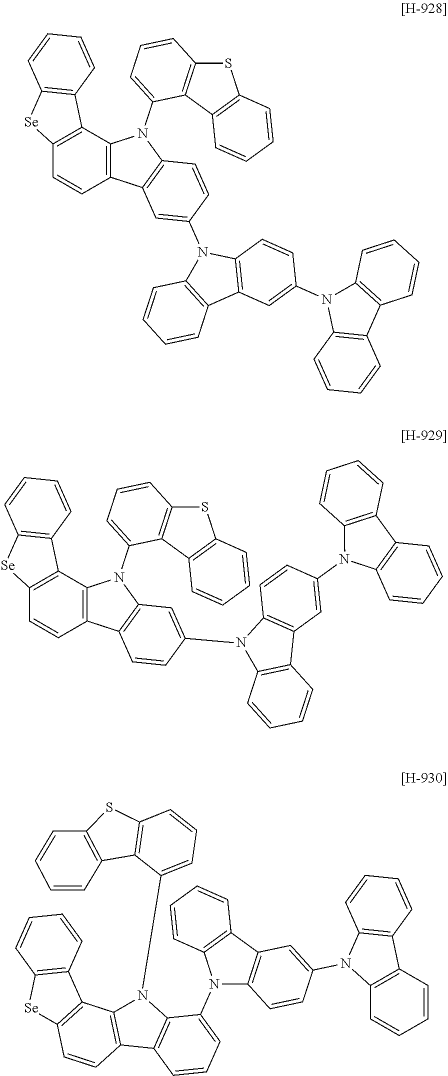 [SMILES: c1ccc2c(c1)sc1cccc(-n3c4ccc(-n5c6ccccc6c6cc(-n7c8ccccc8c8ccccc87)ccc65)cc4c4ccc5[se]c6ccccc6c5c43)c12 ; c1ccc2c(c1)sc1cccc(-n3c4cc(-n5c6ccccc6c6cc(-n7c8ccccc8c8ccccc87)ccc65)ccc4c4ccc5[se]c6ccccc6c5c43)c12 ; c1ccc2c(c1)sc1cccc(-n3c4c(-n5c6ccccc6c6cc(-n7c8ccccc8c8ccccc87)ccc65)cccc4c4ccc5[se]c6ccccc6c5c43)c12]

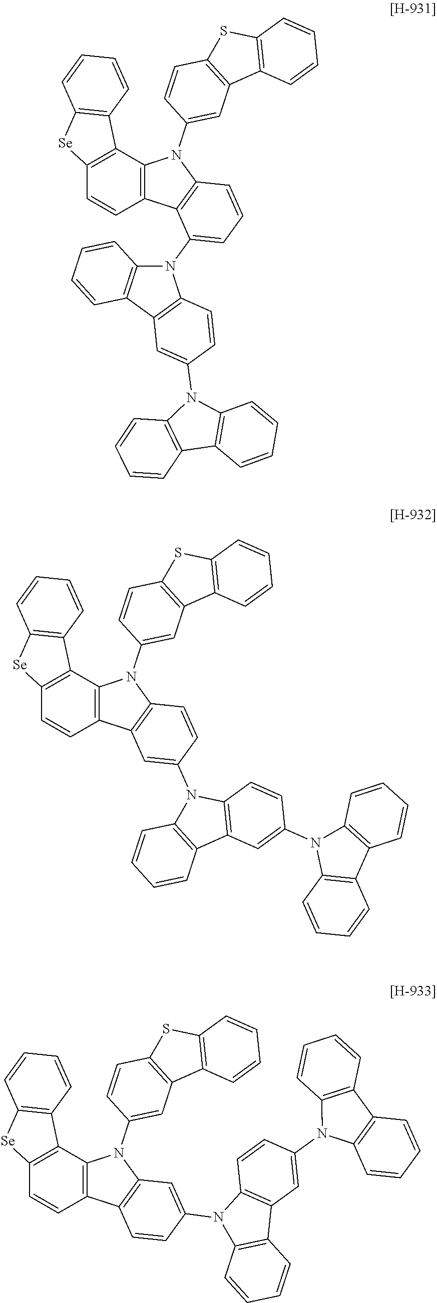 [SMILES: c1ccc2c(c1)sc1ccc(-n3c4cccc(-n5c6ccccc6c6cc(-n7c8ccccc8c8ccccc87)ccc65)c4c4ccc5[se]c6ccccc6c5c43)cc12 ; c1ccc2c(c1)sc1ccc(-n3c4ccc(-n5c6ccccc6c6cc(-n7c8ccccc8c8ccccc87)ccc65)cc4c4ccc5[se]c6ccccc6c5c43)cc12 ; c1ccc2c(c1)sc1ccc(-n3c4cc(-n5c6ccccc6c6cc(-n7c8ccccc8c8ccccc87)ccc65)ccc4c4ccc5[se]c6ccccc6c5c43)cc12]

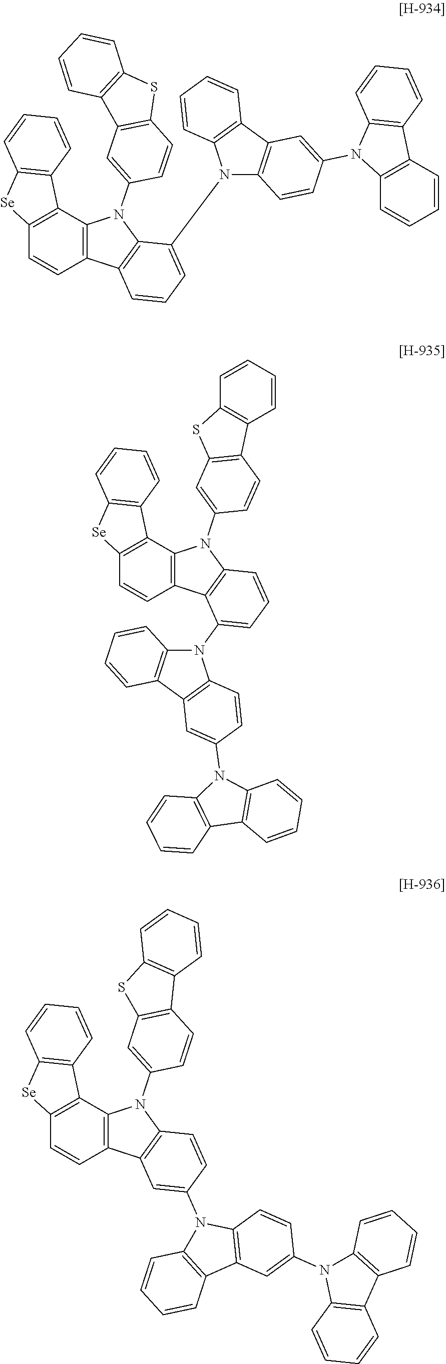 [SMILES: c1ccc2c(c1)sc1ccc(-n3c4c(-n5c6ccccc6c6cc(-n7c8ccccc8c8ccccc87)ccc65)cccc4c4ccc5[se]c6ccccc6c5c43)cc12 ; c1ccc2c(c1)sc1cc(-n3c4cccc(-n5c6ccccc6c6cc(-n7c8ccccc8c8ccccc87)ccc65)c4c4ccc5[se]c6ccccc6c5c43)ccc12 ; c1ccc2c(c1)sc1cc(-n3c4ccc(-n5c6ccccc6c6cc(-n7c8ccccc8c8ccccc87)ccc65)cc4c4ccc5[se]c6ccccc6c5c43)ccc12]

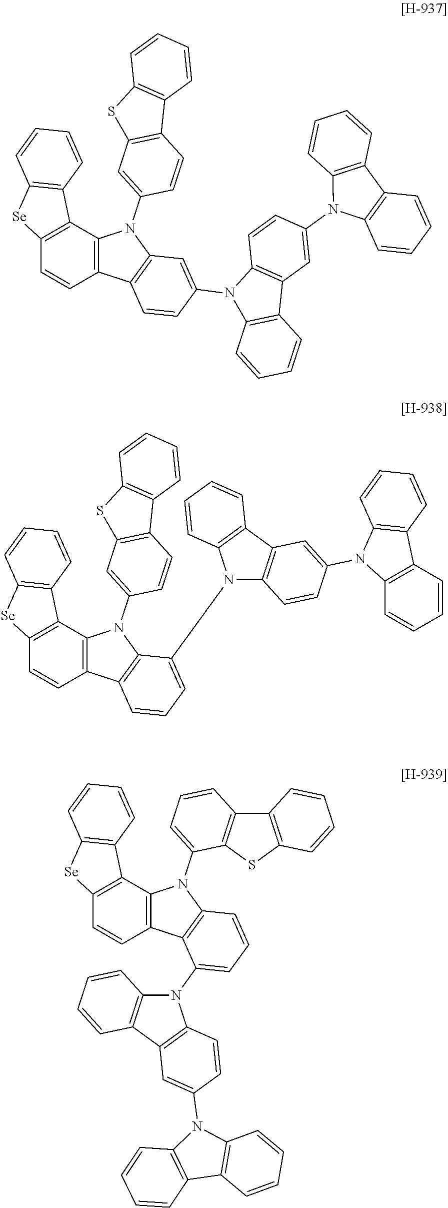 [SMILES: c1ccc2c(c1)sc1cc(-n3c4cc(-n5c6ccccc6c6cc(-n7c8ccccc8c8ccccc87)ccc65)ccc4c4ccc5[se]c6ccccc6c5c43)ccc12 ; c1ccc2c(c1)sc1cc(-n3c4c(-n5c6ccccc6c6cc(-n7c8ccccc8c8ccccc87)ccc65)cccc4c4ccc5[se]c6ccccc6c5c43)ccc12 ; c1ccc2c(c1)sc1c(-n3c4cccc(-n5c6ccccc6c6cc(-n7c8ccccc8c8ccccc87)ccc65)c4c4ccc5[se]c6ccccc6c5c43)cccc12]

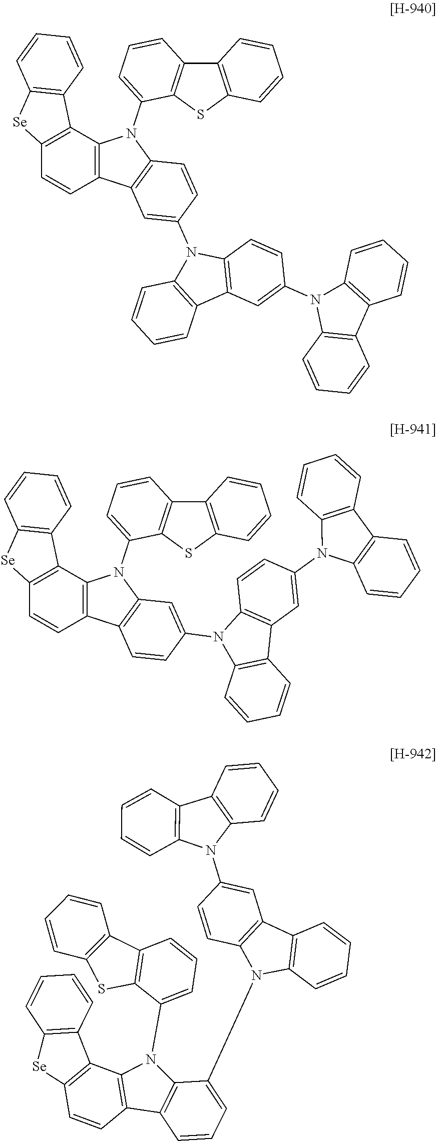

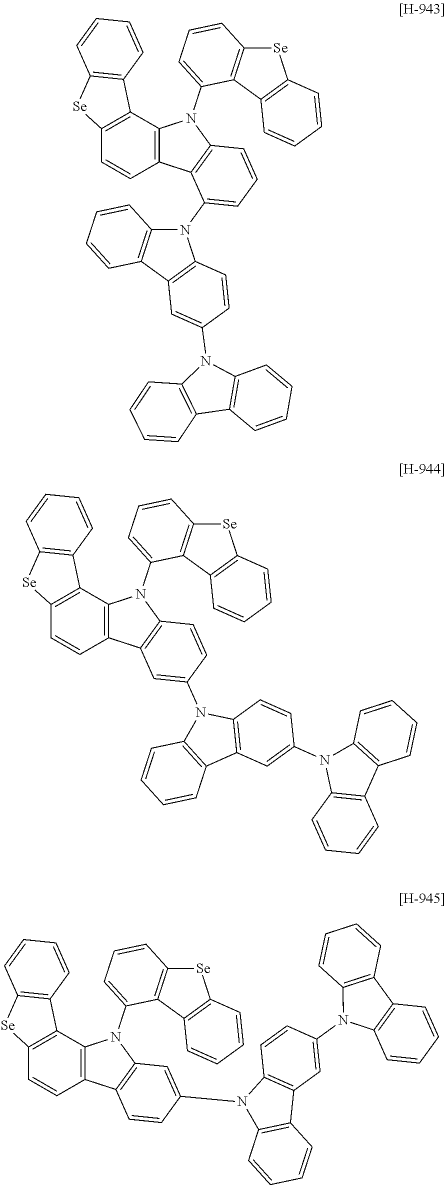 [SMILES: c1ccc2c(c1)[se]c1cccc(-n3c4cccc(-n5c6ccccc6c6cc(-n7c8ccccc8c8ccccc87)ccc65)c4c4ccc5[se]c6ccccc6c5c43)c12 ; c1ccc2c(c1)[se]c1cccc(-n3c4ccc(-n5c6ccccc6c6cc(-n7c8ccccc8c8ccccc87)ccc65)cc4c4ccc5[se]c6ccccc6c5c43)c12 ; c1ccc2c(c1)[se]c1cccc(-n3c4cc(-n5c6ccccc6c6cc(-n7c8ccccc8c8ccccc87)ccc65)ccc4c4ccc5[se]c6ccccc6c5c43)c12]

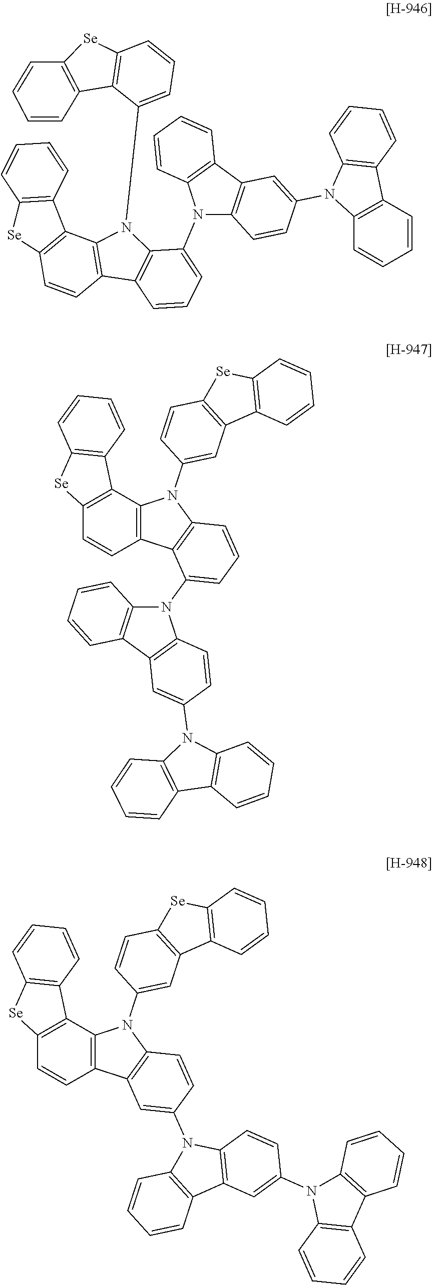 [SMILES: c1ccc2c(c1)[se]c1cccc(-n3c4c(-n5c6ccccc6c6cc(-n7c8ccccc8c8ccccc87)ccc65)cccc4c4ccc5[se]c6ccccc6c5c43)c12 ; c1ccc2c(c1)[se]c1ccc(-n3c4cccc(-n5c6ccccc6c6cc(-n7c8ccccc8c8ccccc87)ccc65)c4c4ccc5[se]c6ccccc6c5c43)cc12 ; c1ccc2c(c1)[se]c1ccc(-n3c4ccc(-n5c6ccccc6c6cc(-n7c8ccccc8c8ccccc87)ccc65)cc4c4ccc5[se]c6ccccc6c5c43)cc12]

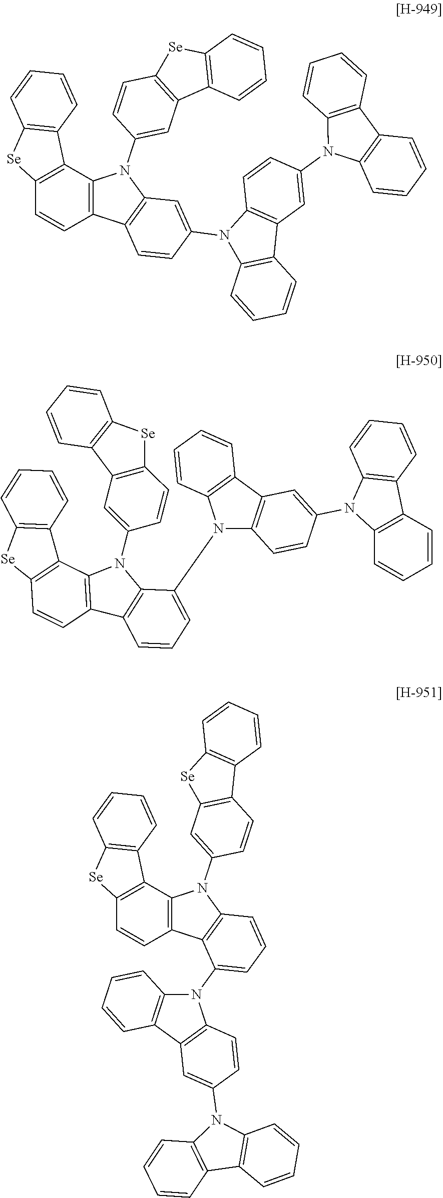 [SMILES: c1ccc2c(c1)[se]c1ccc(-n3c4cc(-n5c6ccccc6c6cc(-n7c8ccccc8c8ccccc87)ccc65)ccc4c4ccc5[se]c6ccccc6c5c43)cc12 ; c1ccc2c(c1)[se]c1ccc(-n3c4c(-n5c6ccccc6c6cc(-n7c8ccccc8c8ccccc87)ccc65)cccc4c4ccc5[se]c6ccccc6c5c43)cc12 ; c1ccc2c(c1)[se]c1cc(-n3c4cccc(-n5c6ccccc6c6cc(-n7c8ccccc8c8ccccc87)ccc65)c4c4ccc5[se]c6ccccc6c5c43)ccc12]

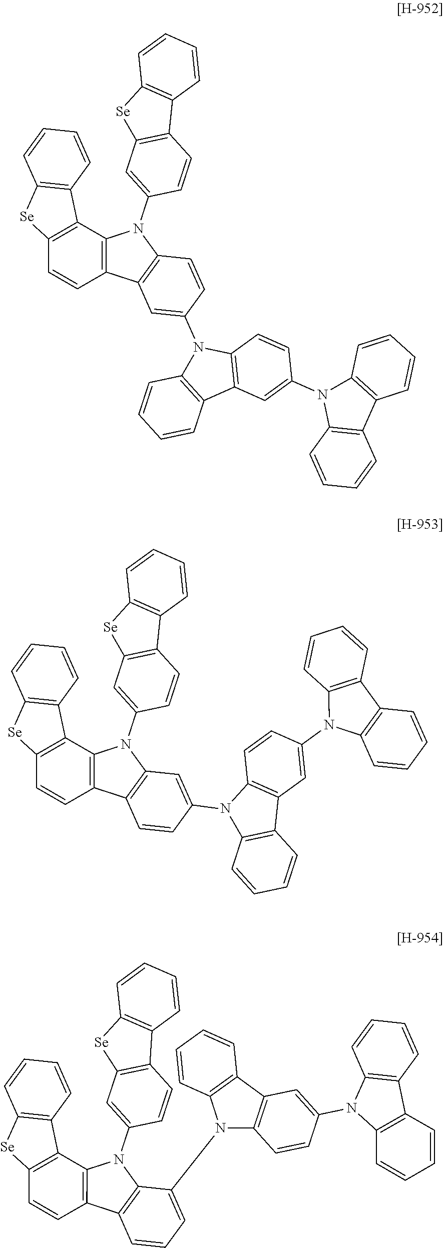 [SMILES: c1ccc2c(c1)[se]c1cc(-n3c4ccc(-n5c6ccccc6c6cc(-n7c8ccccc8c8ccccc87)ccc65)cc4c4ccc5[se]c6ccccc6c5c43)ccc12 ; c1ccc2c(c1)[se]c1cc(-n3c4cc(-n5c6ccccc6c6cc(-n7c8ccccc8c8ccccc87)ccc65)ccc4c4ccc5[se]c6ccccc6c5c43)ccc12 ; c1ccc2c(c1)[se]c1cc(-n3c4c(-n5c6ccccc6c6cc(-n7c8ccccc8c8ccccc87)ccc65)cccc4c4ccc5[se]c6ccccc6c5c43)ccc12]

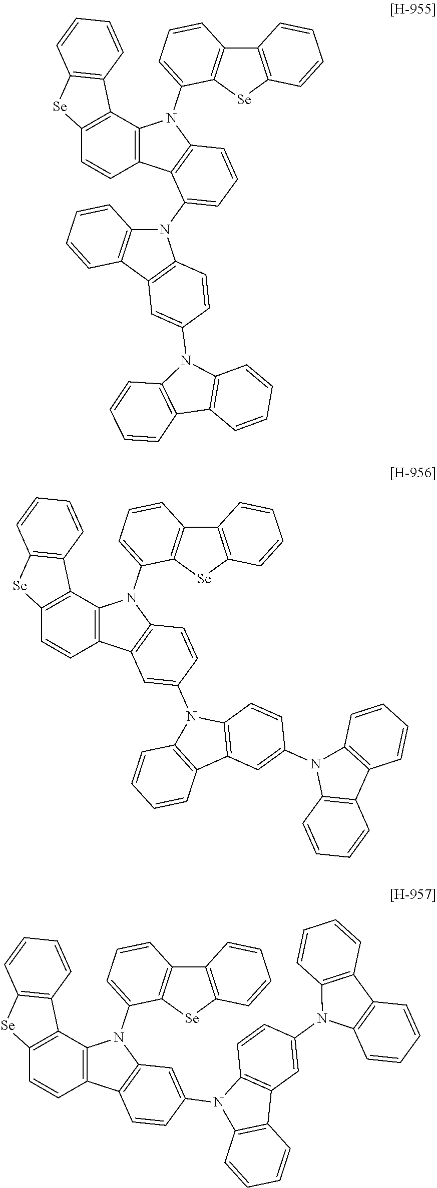 [SMILES: c1ccc2c(c1)[se]c1c(-n3c4cccc(-n5c6ccccc6c6cc(-n7c8ccccc8c8ccccc87)ccc65)c4c4ccc5[se]c6ccccc6c5c43)cccc12 ; c1ccc2c(c1)[se]c1c(-n3c4ccc(-n5c6ccccc6c6cc(-n7c8ccccc8c8ccccc87)ccc65)cc4c4ccc5[se]c6ccccc6c5c43)cccc12 ; c1ccc2c(c1)[se]c1c(-n3c4cc(-n5c6ccccc6c6cc(-n7c8ccccc8c8ccccc87)ccc65)ccc4c4ccc5[se]c6ccccc6c5c43)cccc12]

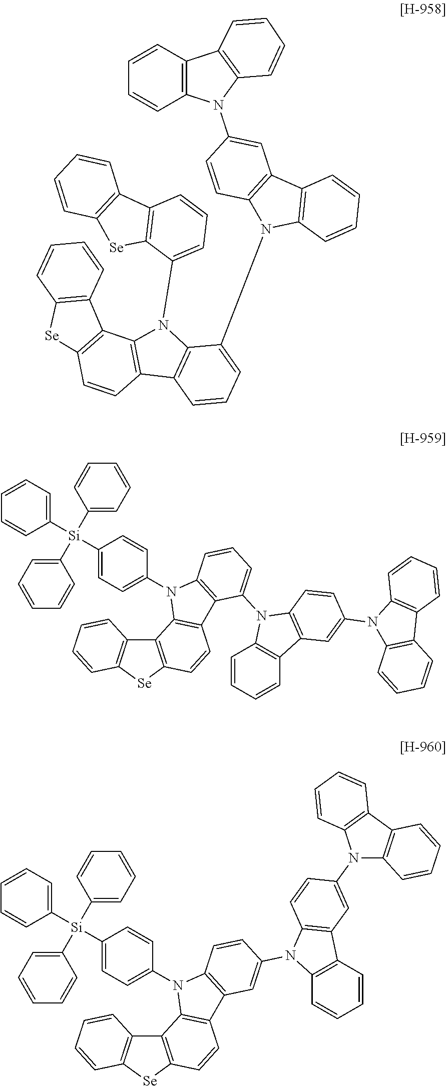 [SMILES: c1ccc2c(c1)[se]c1c(-n3c4c(-n5c6ccccc6c6cc(-n7c8ccccc8c8ccccc87)ccc65)cccc4c4ccc5[se]c6ccccc6c5c43)cccc12 ; c1ccc([Si](c2ccccc2)(c2ccccc2)c2ccc(-n3c4cccc(-n5c6ccccc6c6cc(-n7c8ccccc8c8ccccc87)ccc65)c4c4ccc5[se]c6ccccc6c5c43)cc2)cc1 ; c1ccc([Si](c2ccccc2)(c2ccccc2)c2ccc(-n3c4ccc(-n5c6ccccc6c6cc(-n7c8ccccc8c8ccccc87)ccc65)cc4c4ccc5[se]c6ccccc6c5c43)cc2)cc1]

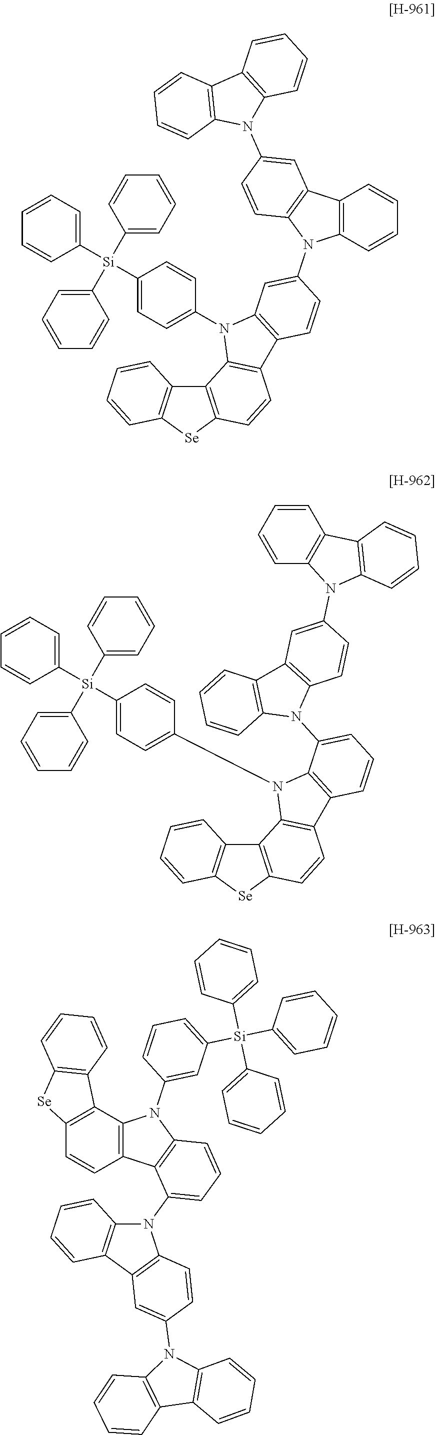 [SMILES: c1ccc([Si](c2ccccc2)(c2ccccc2)c2ccc(-n3c4cc(-n5c6ccccc6c6cc(-n7c8ccccc8c8ccccc87)ccc65)ccc4c4ccc5[se]c6ccccc6c5c43)cc2)cc1 ; c1ccc([Si](c2ccccc2)(c2ccccc2)c2ccc(-n3c4c(-n5c6ccccc6c6cc(-n7c8ccccc8c8ccccc87)ccc65)cccc4c4ccc5[se]c6ccccc6c5c43)cc2)cc1 ; c1ccc([Si](c2ccccc2)(c2ccccc2)c2cccc(-n3c4cccc(-n5c6ccccc6c6cc(-n7c8ccccc8c8ccccc87)ccc65)c4c4ccc5[se]c6ccccc6c5c43)c2)cc1]

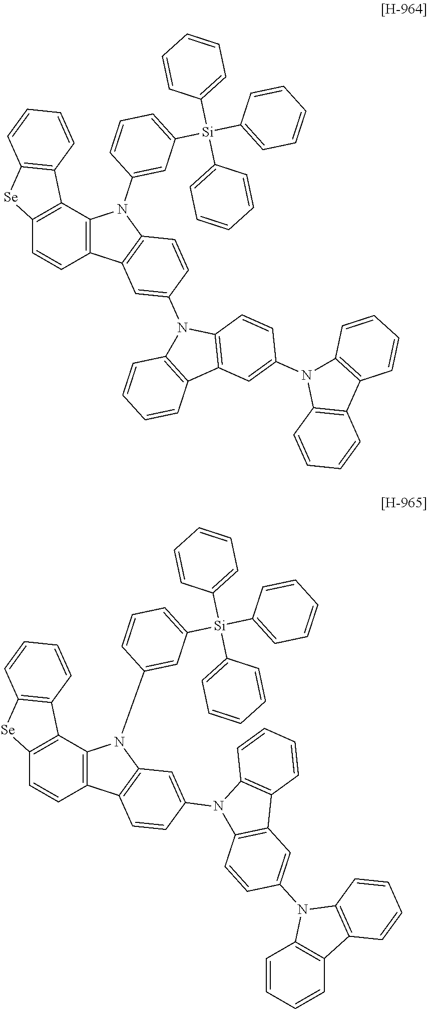 [SMILES: c1ccc([Si](c2ccccc2)(c2ccccc2)c2cccc(-n3c4ccc(-n5c6ccccc6c6cc(-n7c8ccccc8c8ccccc87)ccc65)cc4c4ccc5[se]c6ccccc6c5c43)c2)cc1 ; c1ccc([Si](c2ccccc2)(c2ccccc2)c2cccc(-n3c4cc(-n5c6ccccc6c6cc(-n7c8ccccc8c8ccccc87)ccc65)ccc4c4ccc5[se]c6ccccc6c5c43)c2)cc1]

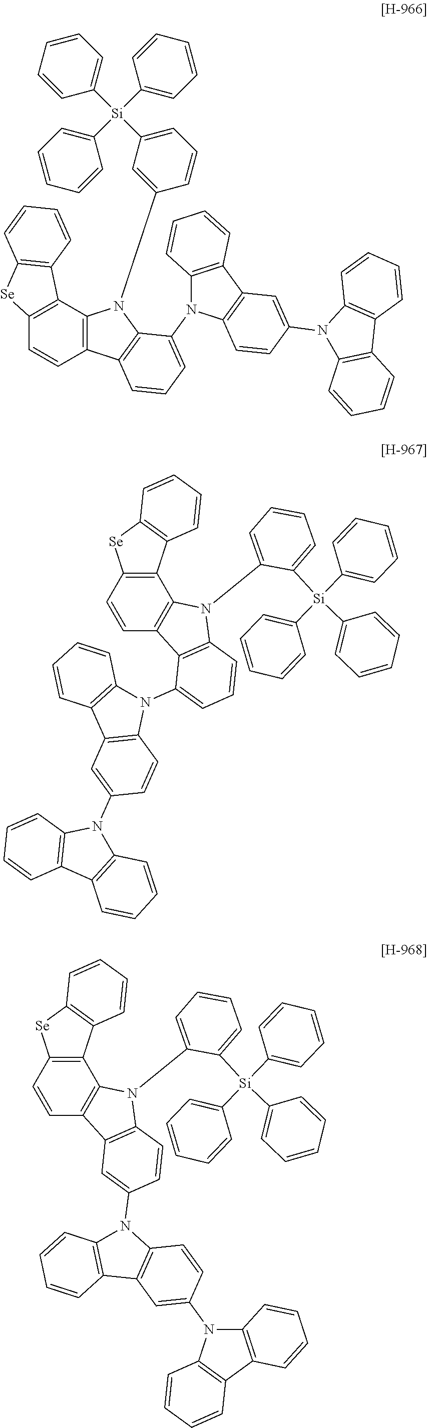 [SMILES: c1ccc([Si](c2ccccc2)(c2ccccc2)c2cccc(-n3c4c(-n5c6ccccc6c6cc(-n7c8ccccc8c8ccccc87)ccc65)cccc4c4ccc5[se]c6ccccc6c5c43)c2)cc1 ; c1ccc([Si](c2ccccc2)(c2ccccc2)c2ccccc2-n2c3cccc(-n4c5ccccc5c5cc(-n6c7ccccc7c7ccccc76)ccc54)c3c3ccc4[se]c5ccccc5c4c32)cc1 ; c1ccc([Si](c2ccccc2)(c2ccccc2)c2ccccc2-n2c3ccc(-n4c5ccccc5c5cc(-n6c7ccccc7c7ccccc76)ccc54)cc3c3ccc4[se]c5ccccc5c4c32)cc1]

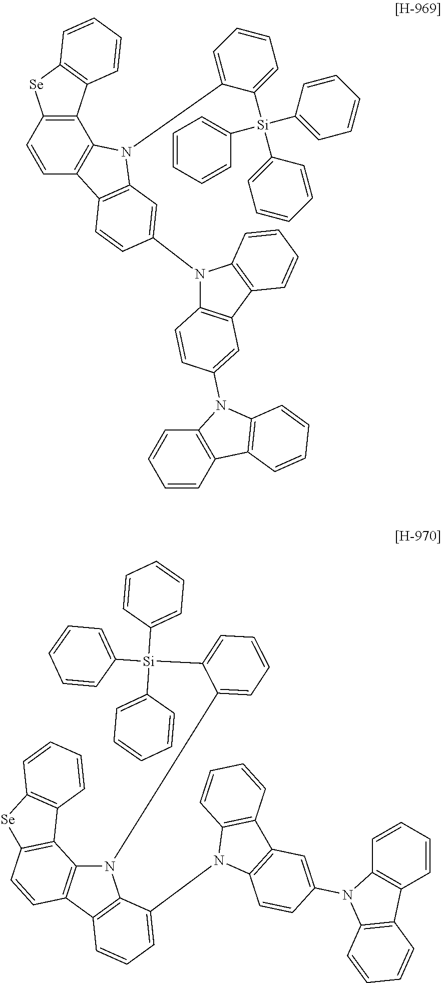 [SMILES: c1ccc([Si](c2ccccc2)(c2ccccc2)c2ccccc2-n2c3cc(-n4c5ccccc5c5cc(-n6c7ccccc7c7ccccc76)ccc54)ccc3c3ccc4[se]c5ccccc5c4c32)cc1 ; c1ccc([Si](c2ccccc2)(c2ccccc2)c2ccccc2-n2c3c(-n4c5ccccc5c5cc(-n6c7ccccc7c7ccccc76)ccc54)cccc3c3ccc4[se]c5ccccc5c4c32)cc1]

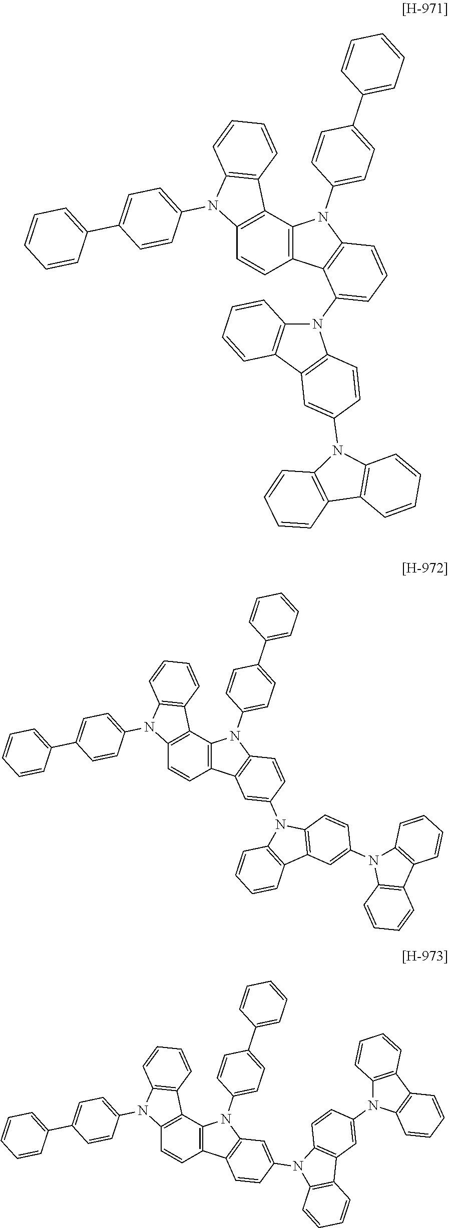 [SMILES: c1ccc(-c2ccc(-n3c4ccccc4c4c3ccc3c5c(-n6c7ccccc7c7cc(-n8c9ccccc9c9ccccc98)ccc76)cccc5n(-c5ccc(-c6ccccc6)cc5)c34)cc2)cc1 ; c1ccc(-c2ccc(-n3c4ccccc4c4c3ccc3c5cc(-n6c7ccccc7c7cc(-n8c9ccccc9c9ccccc98)ccc76)ccc5n(-c5ccc(-c6ccccc6)cc5)c34)cc2)cc1 ; c1ccc(-c2ccc(-n3c4ccccc4c4c3ccc3c5ccc(-n6c7ccccc7c7cc(-n8c9ccccc9c9ccccc98)ccc76)cc5n(-c5ccc(-c6ccccc6)cc5)c34)cc2)cc1]

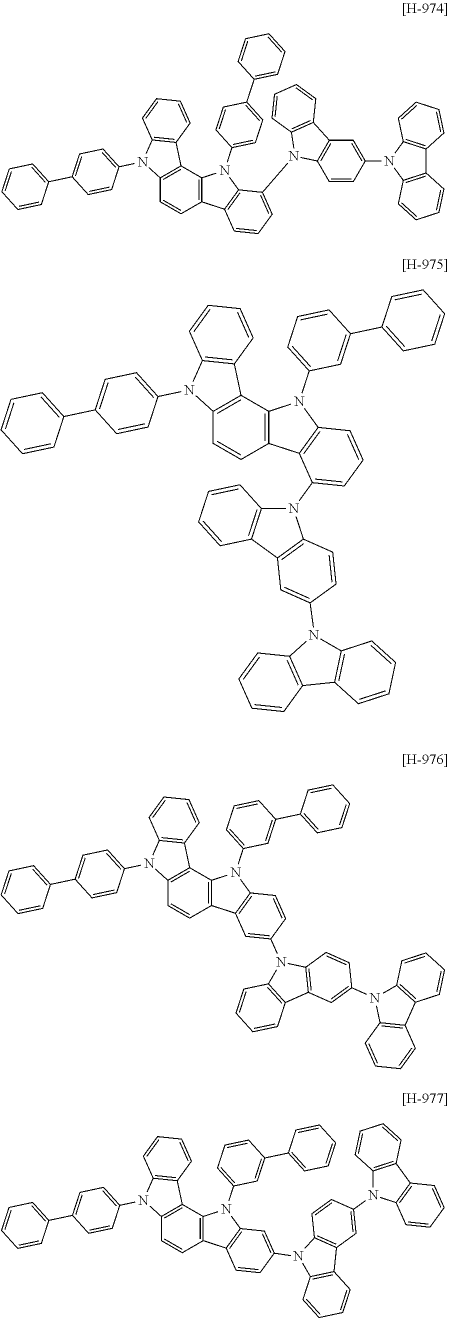 [SMILES: c1ccc(-c2ccc(-n3c4ccccc4c4c3ccc3c5cccc(-n6c7ccccc7c7cc(-n8c9ccccc9c9ccccc98)ccc76)c5n(-c5ccc(-c6ccccc6)cc5)c34)cc2)cc1 ; c1ccc(-c2ccc(-n3c4ccccc4c4c3ccc3c5c(-n6c7ccccc7c7cc(-n8c9ccccc9c9ccccc98)ccc76)cccc5n(-c5cccc(-c6ccccc6)c5)c34)cc2)cc1 ; c1ccc(-c2ccc(-n3c4ccccc4c4c3ccc3c5cc(-n6c7ccccc7c7cc(-n8c9ccccc9c9ccccc98)ccc76)ccc5n(-c5cccc(-c6ccccc6)c5)c34)cc2)cc1 ; c1ccc(-c2ccc(-n3c4ccccc4c4c3ccc3c5ccc(-n6c7ccccc7c7cc(-n8c9ccccc9c9ccccc98)ccc76)cc5n(-c5cccc(-c6ccccc6)c5)c34)cc2)cc1]

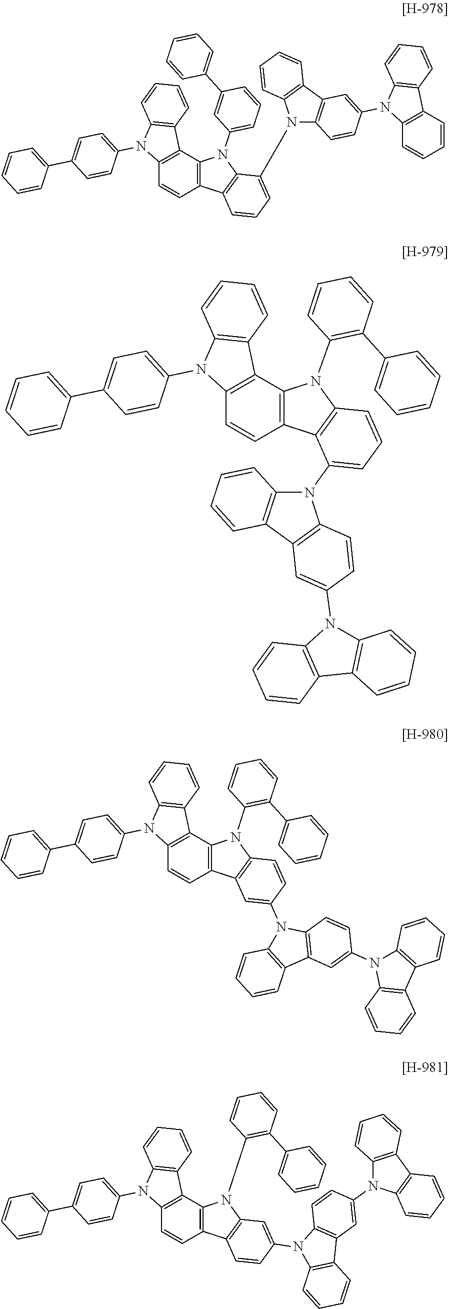 [SMILES: c1ccc(-c2ccc(-n3c4ccccc4c4c3ccc3c5cccc(-n6c7ccccc7c7cc(-n8c9ccccc9c9ccccc98)ccc76)c5n(-c5cccc(-c6ccccc6)c5)c34)cc2)cc1 ; c1ccc(-c2ccc(-n3c4ccccc4c4c3ccc3c5c(-n6c7ccccc7c7cc(-n8c9ccccc9c9ccccc98)ccc76)cccc5n(-c5ccccc5-c5ccccc5)c34)cc2)cc1 ; c1ccc(-c2ccc(-n3c4ccccc4c4c3ccc3c5cc(-n6c7ccccc7c7cc(-n8c9ccccc9c9ccccc98)ccc76)ccc5n(-c5ccccc5-c5ccccc5)c34)cc2)cc1 ; c1ccc(-c2ccc(-n3c4ccccc4c4c3ccc3c5ccc(-n6c7ccccc7c7cc(-n8c9ccccc9c9ccccc98)ccc76)cc5n(-c5ccccc5-c5ccccc5)c34)cc2)cc1]

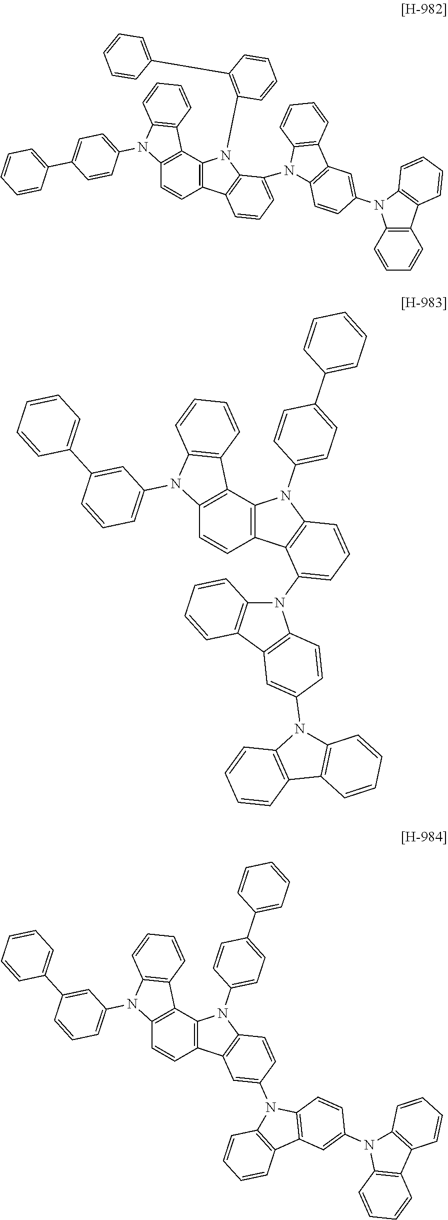 [SMILES: c1ccc(-c2ccc(-n3c4ccccc4c4c3ccc3c5cccc(-n6c7ccccc7c7cc(-n8c9ccccc9c9ccccc98)ccc76)c5n(-c5ccccc5-c5ccccc5)c34)cc2)cc1 ; c1ccc(-c2ccc(-n3c4cccc(-n5c6ccccc6c6cc(-n7c8ccccc8c8ccccc87)ccc65)c4c4ccc5c(c6ccccc6n5-c5cccc(-c6ccccc6)c5)c43)cc2)cc1 ; c1ccc(-c2ccc(-n3c4ccc(-n5c6ccccc6c6cc(-n7c8ccccc8c8ccccc87)ccc65)cc4c4ccc5c(c6ccccc6n5-c5cccc(-c6ccccc6)c5)c43)cc2)cc1]

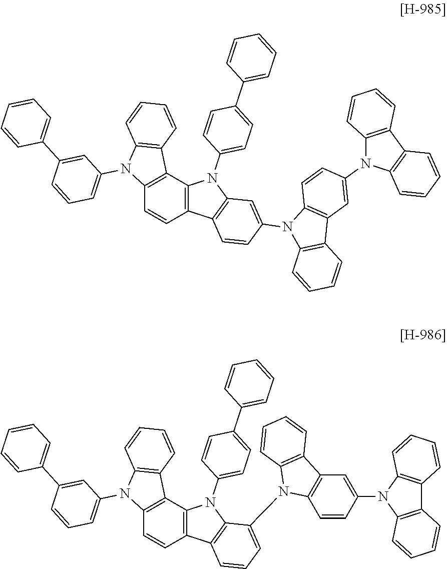 [SMILES: c1ccc(-c2ccc(-n3c4cc(-n5c6ccccc6c6cc(-n7c8ccccc8c8ccccc87)ccc65)ccc4c4ccc5c(c6ccccc6n5-c5cccc(-c6ccccc6)c5)c43)cc2)cc1 ; c1ccc(-c2ccc(-n3c4c(-n5c6ccccc6c6cc(-n7c8ccccc8c8ccccc87)ccc65)cccc4c4ccc5c(c6ccccc6n5-c5cccc(-c6ccccc6)c5)c43)cc2)cc1]

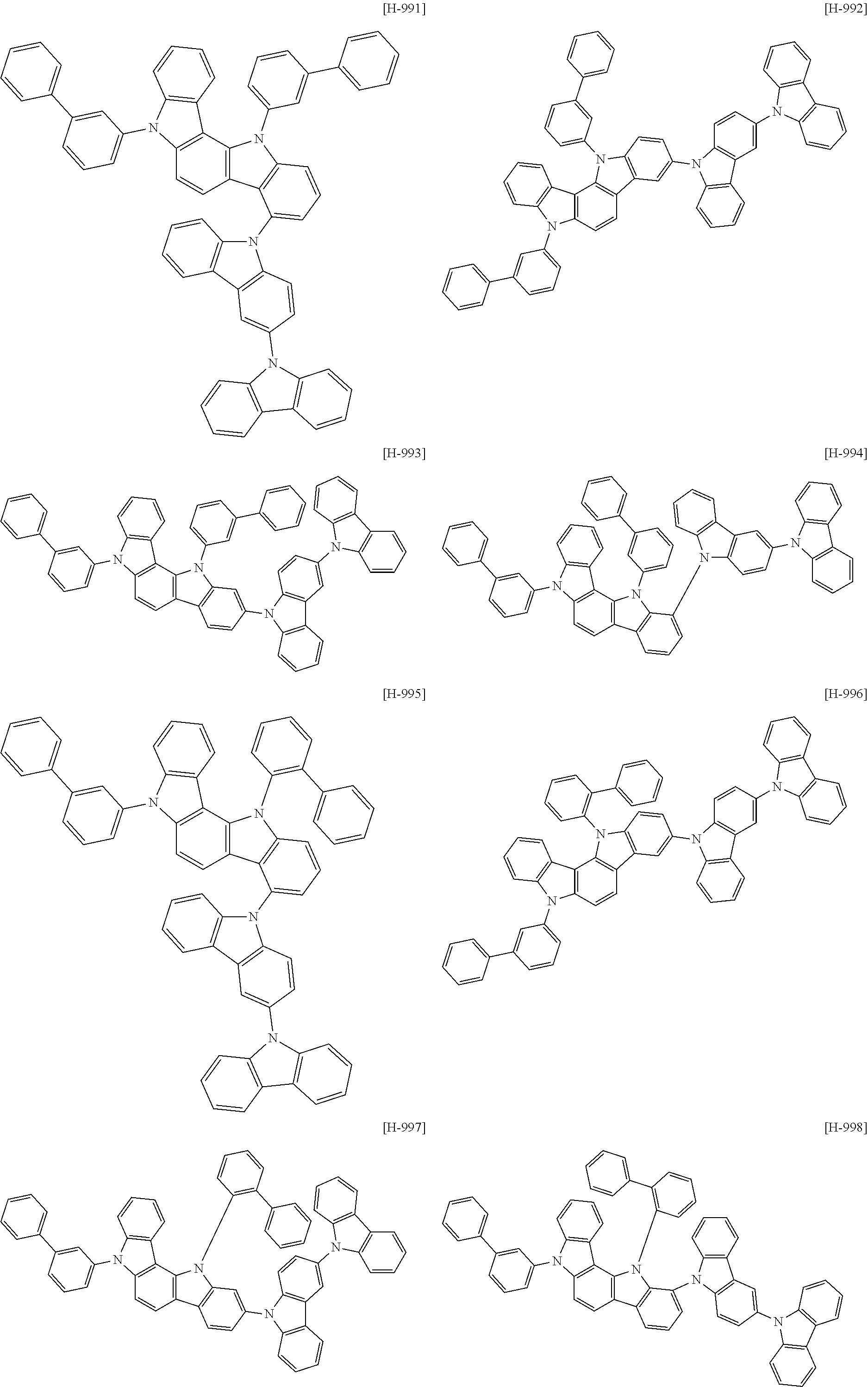

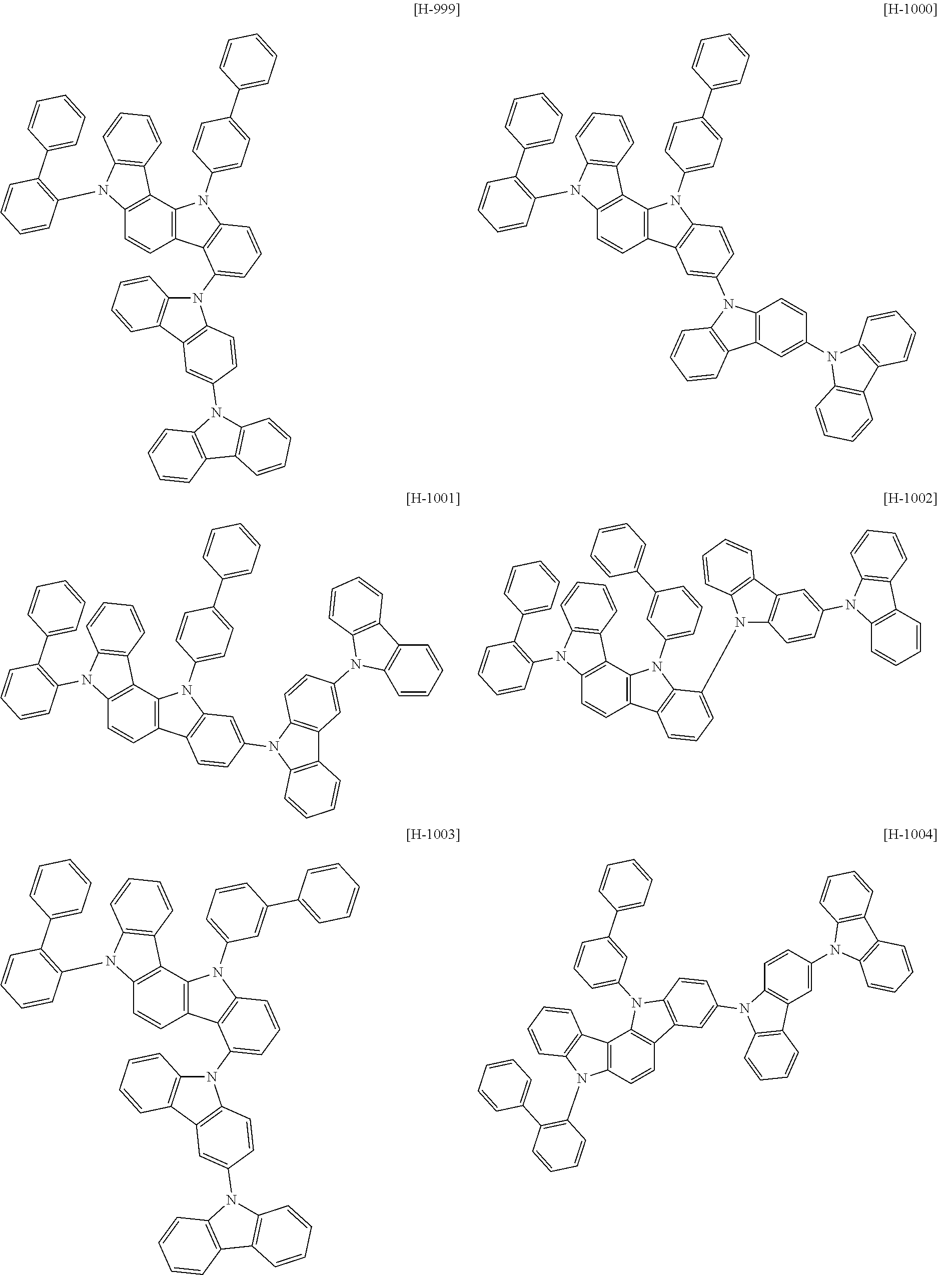

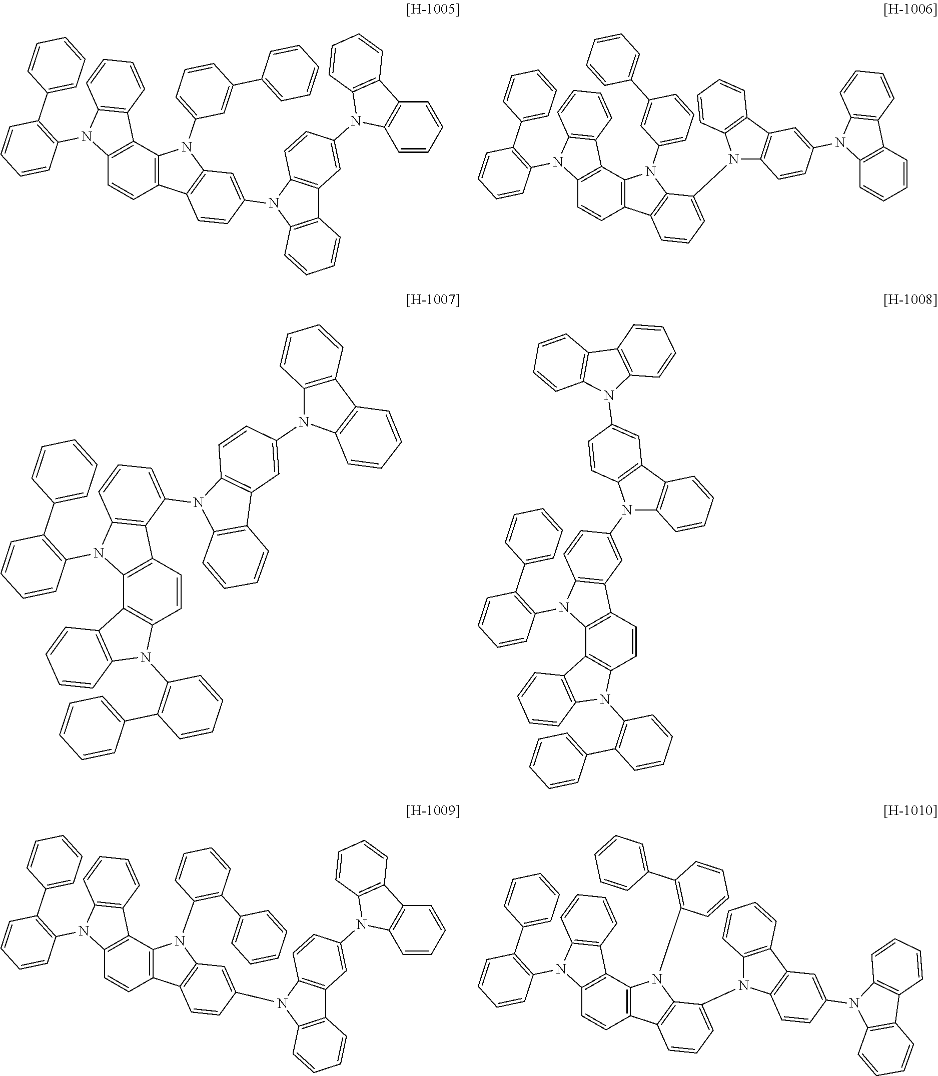

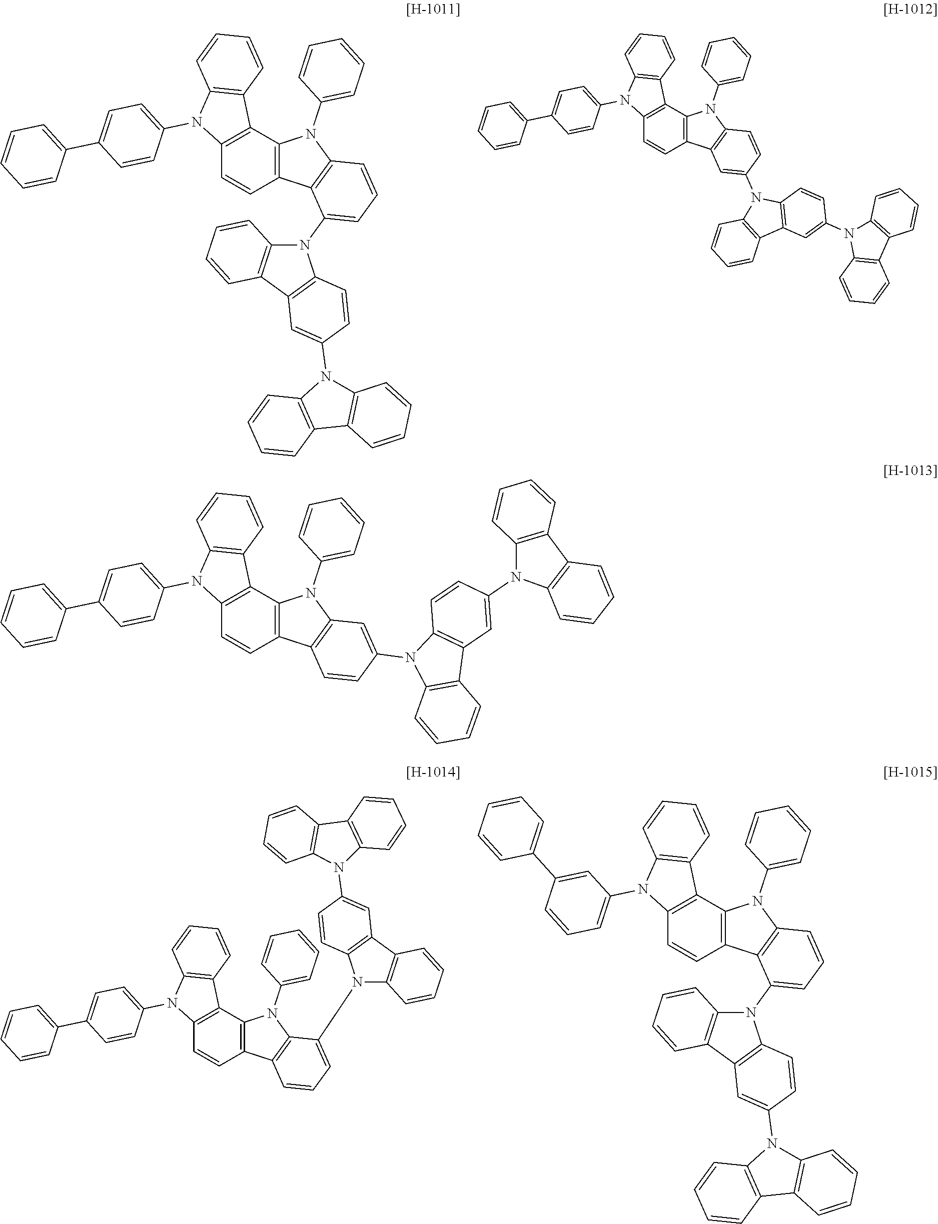

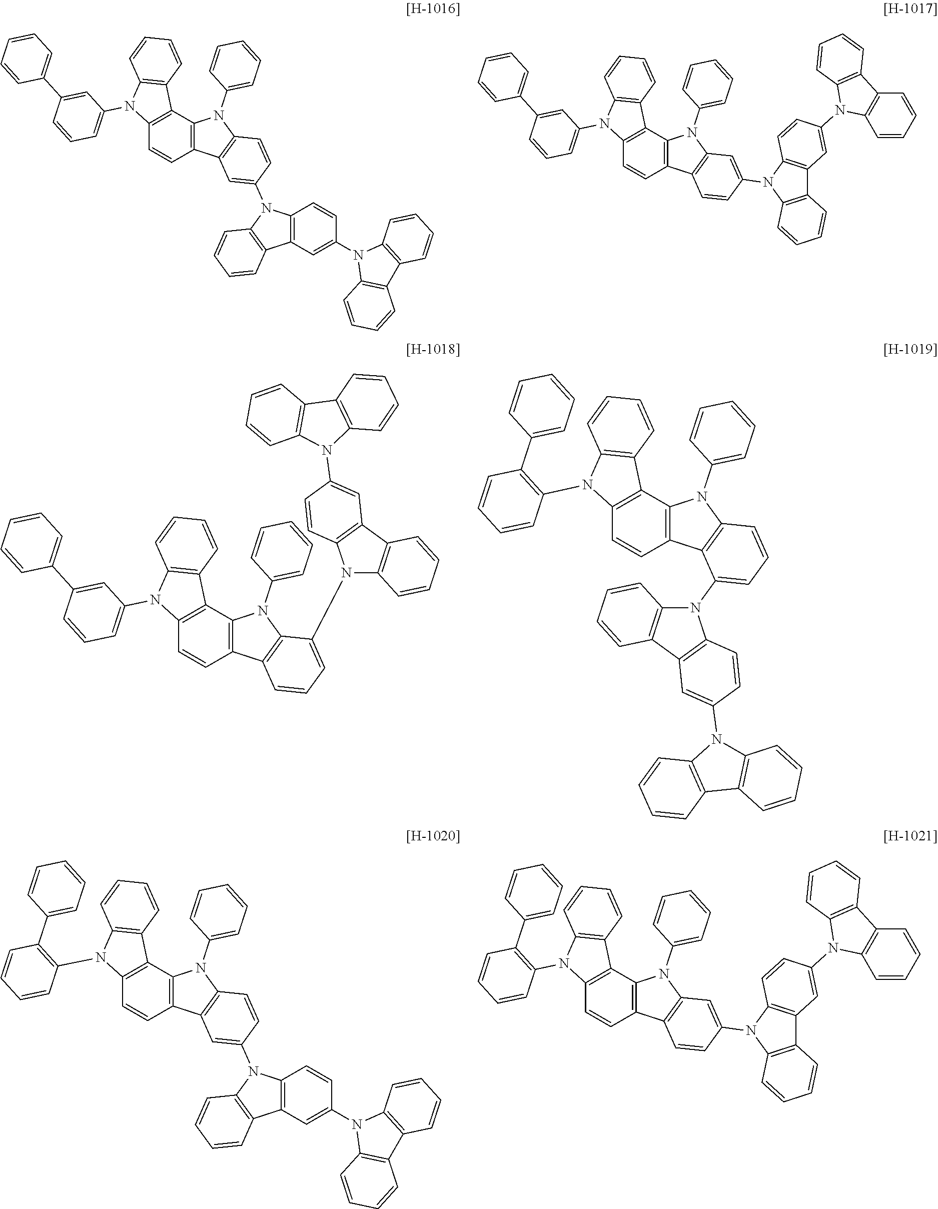

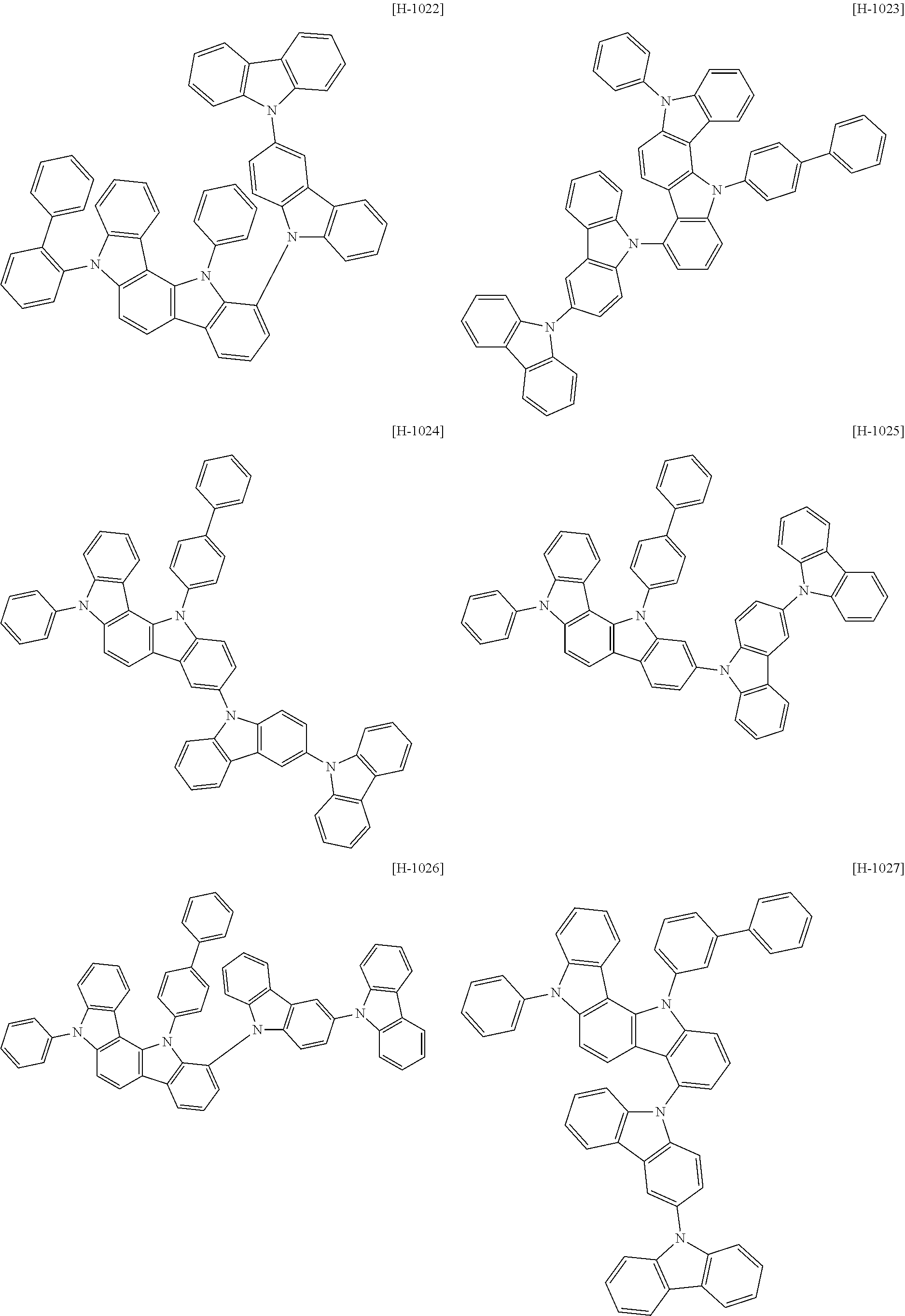

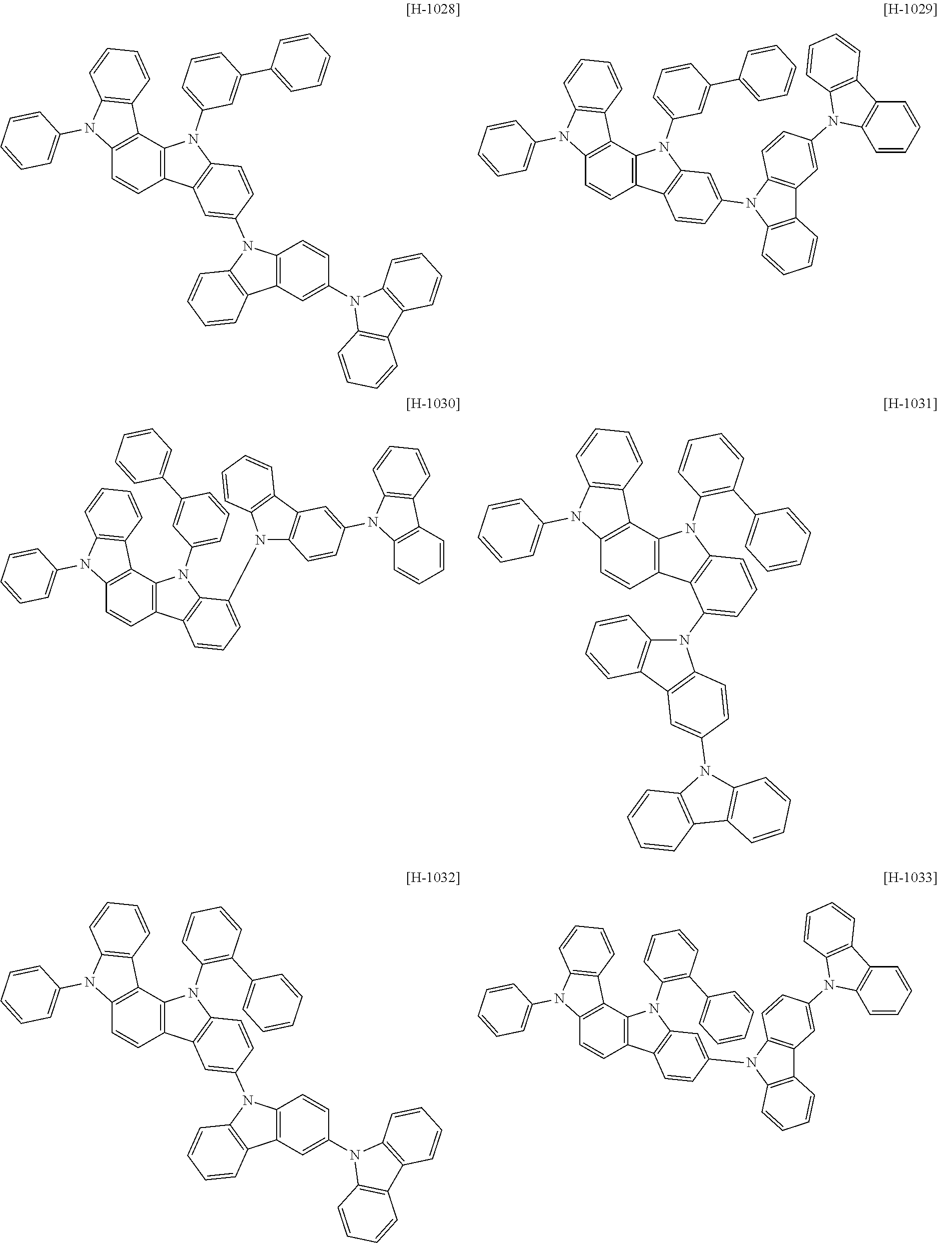

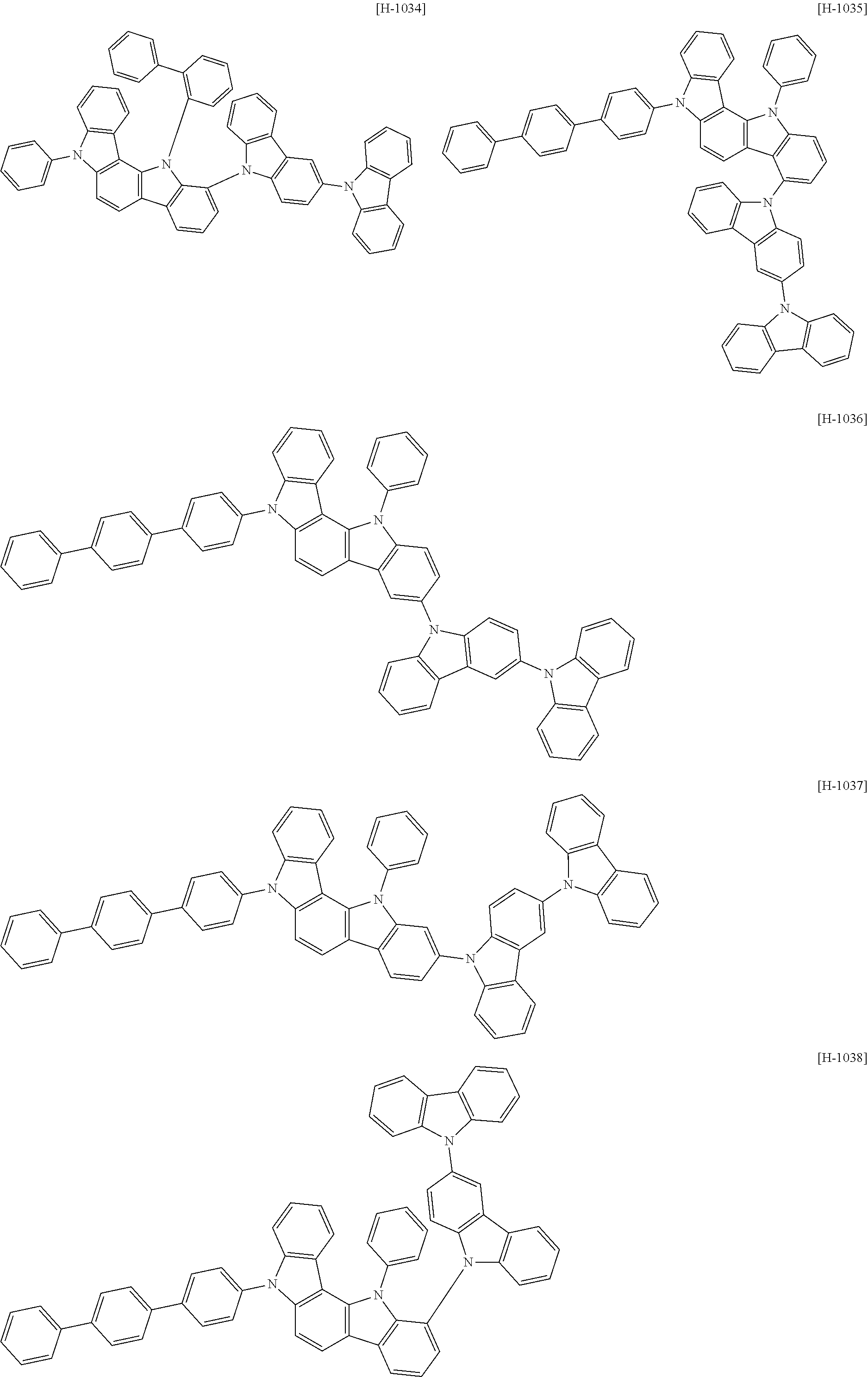

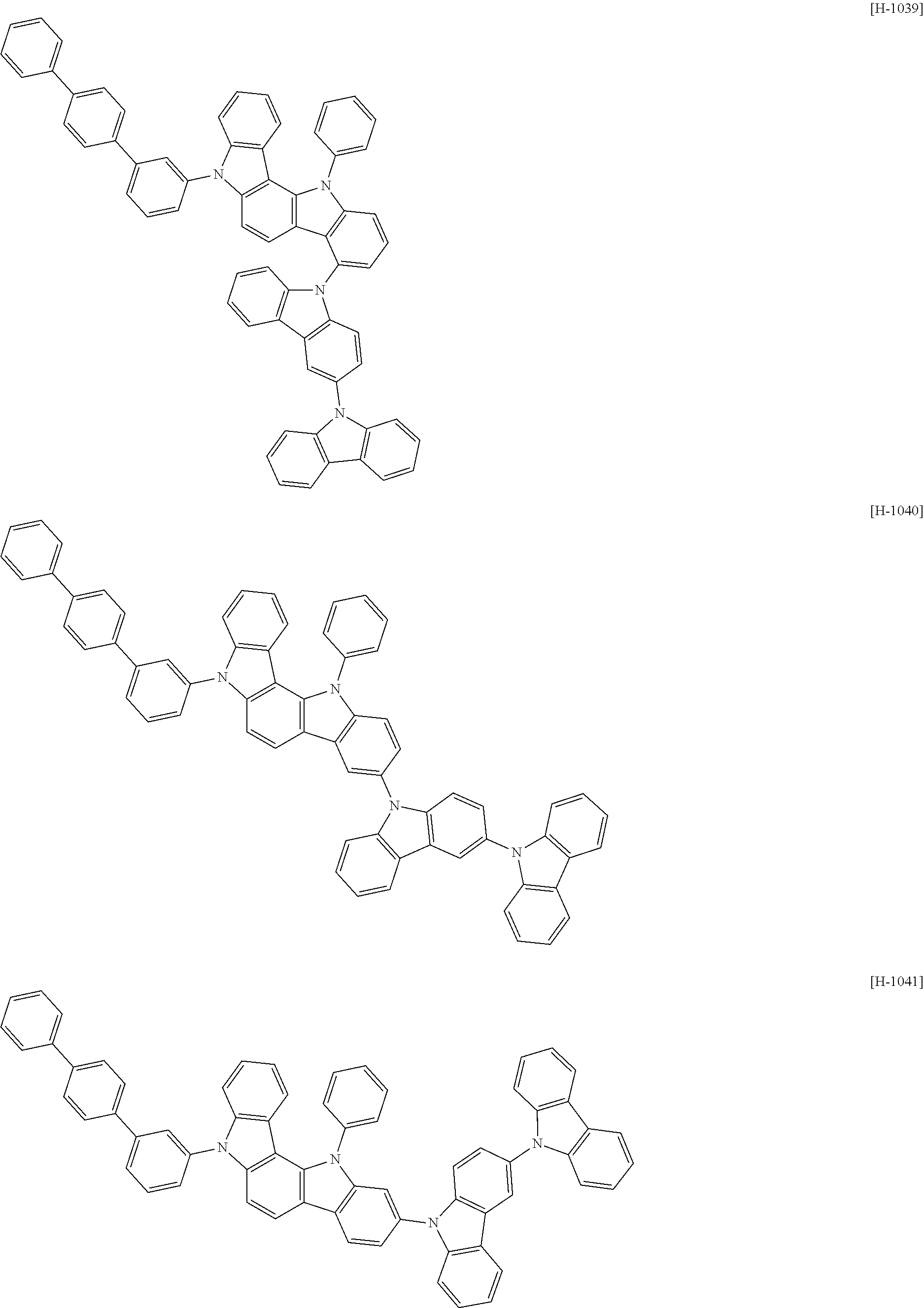 [SMILES: c1ccc(-c2ccc(-c3cccc(-n4c5ccccc5c5c4ccc4c6c(-n7c8ccccc8c8cc(-n9c%10ccccc%10c%10ccccc%109)ccc87)cccc6n(-c6ccccc6)c45)c3)cc2)cc1 ; c1ccc(-c2ccc(-c3cccc(-n4c5ccccc5c5c4ccc4c6cc(-n7c8ccccc8c8cc(-n9c%10ccccc%10c%10ccccc%109)ccc87)ccc6n(-c6ccccc6)c45)c3)cc2)cc1 ; c1ccc(-c2ccc(-c3cccc(-n4c5ccccc5c5c4ccc4c6ccc(-n7c8ccccc8c8cc(-n9c%10ccccc%10c%10ccccc%109)ccc87)cc6n(-c6ccccc6)c45)c3)cc2)cc1]

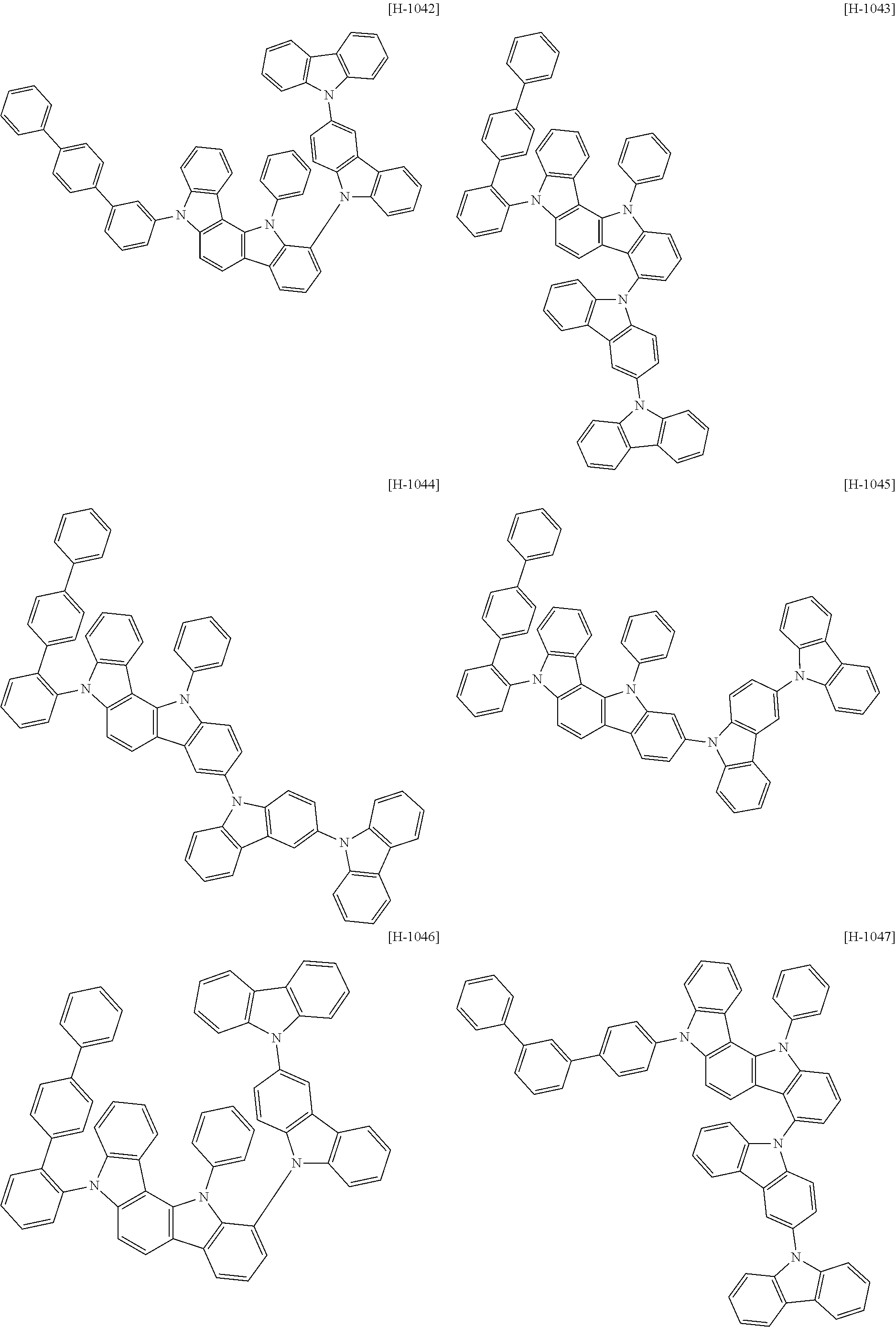

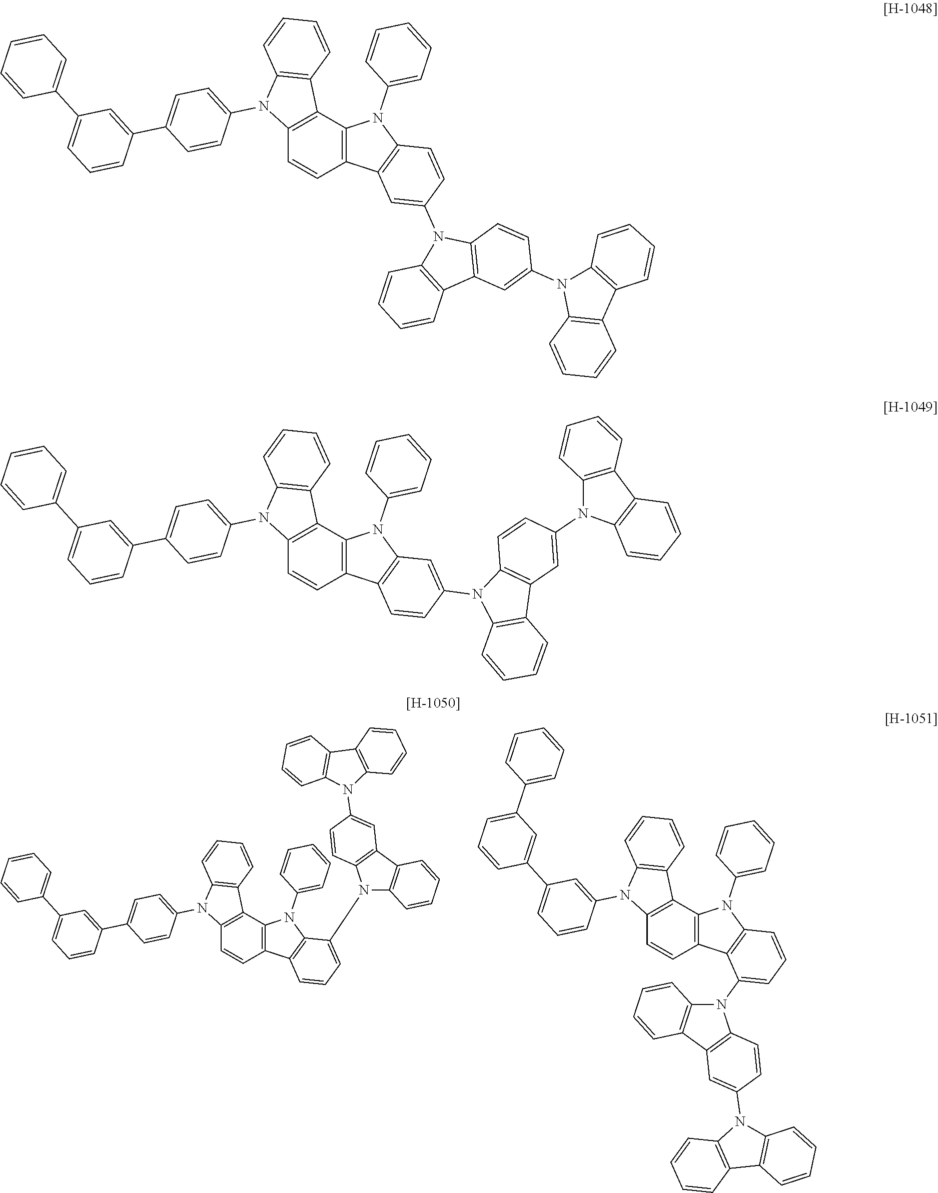

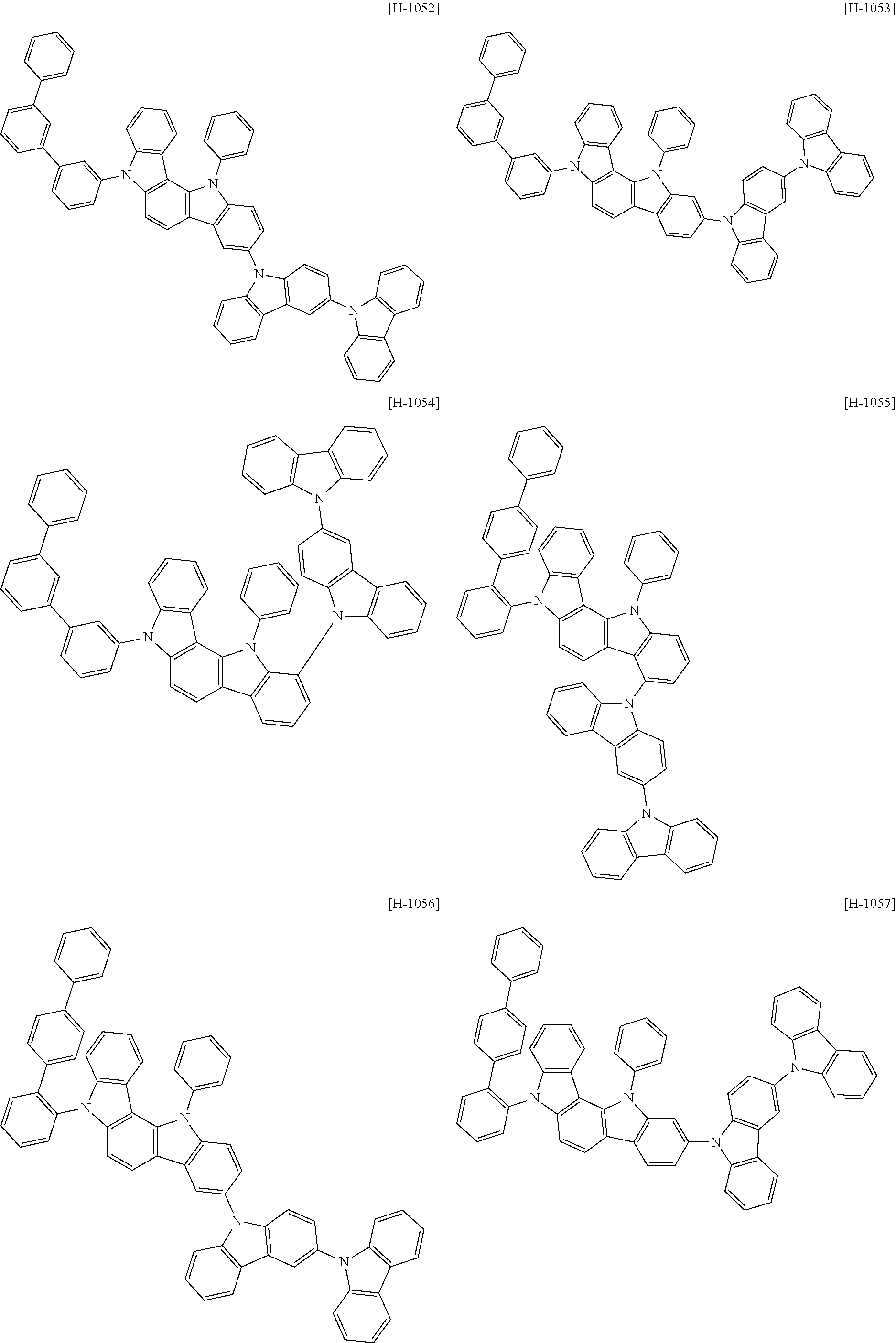

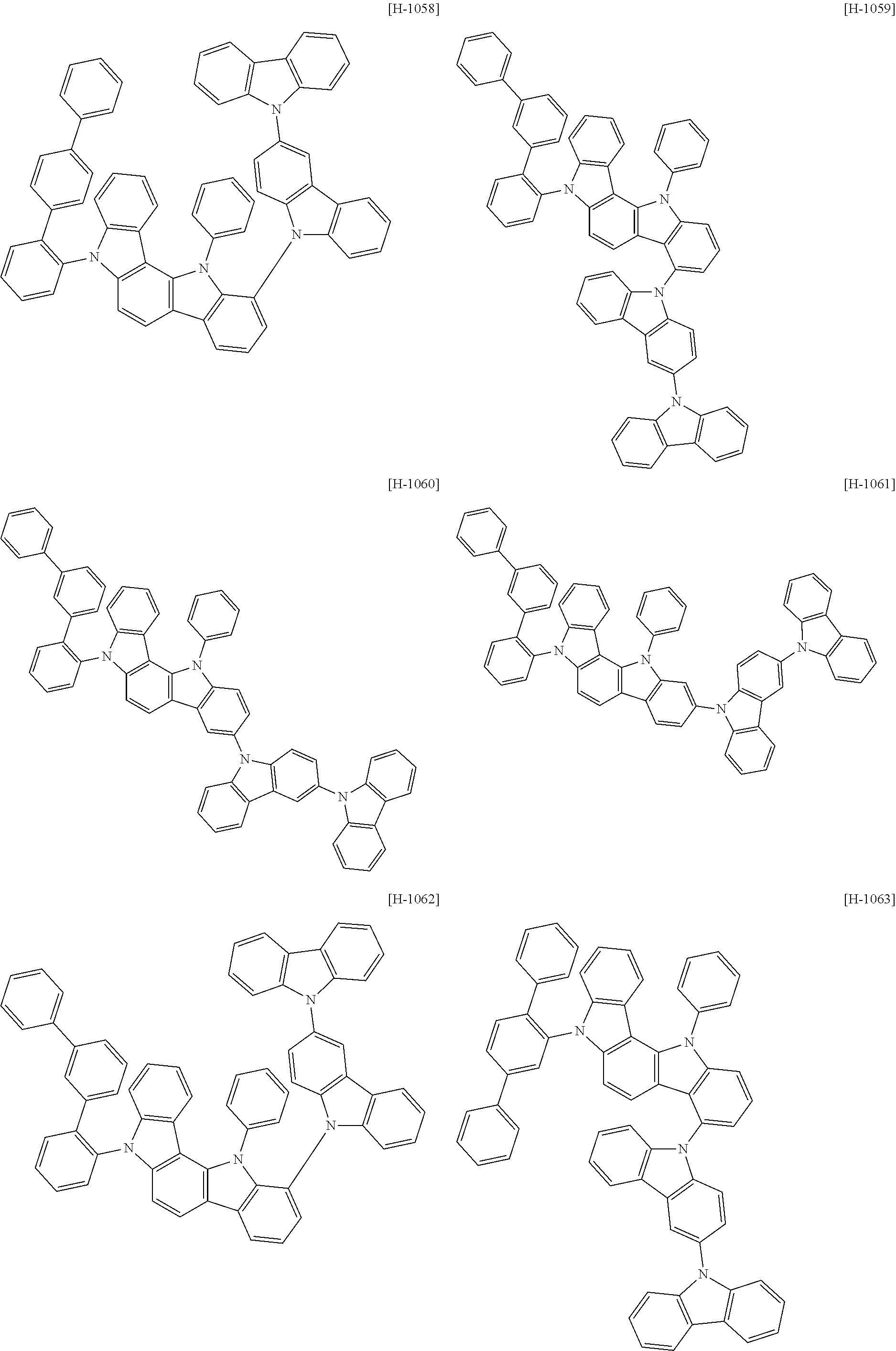

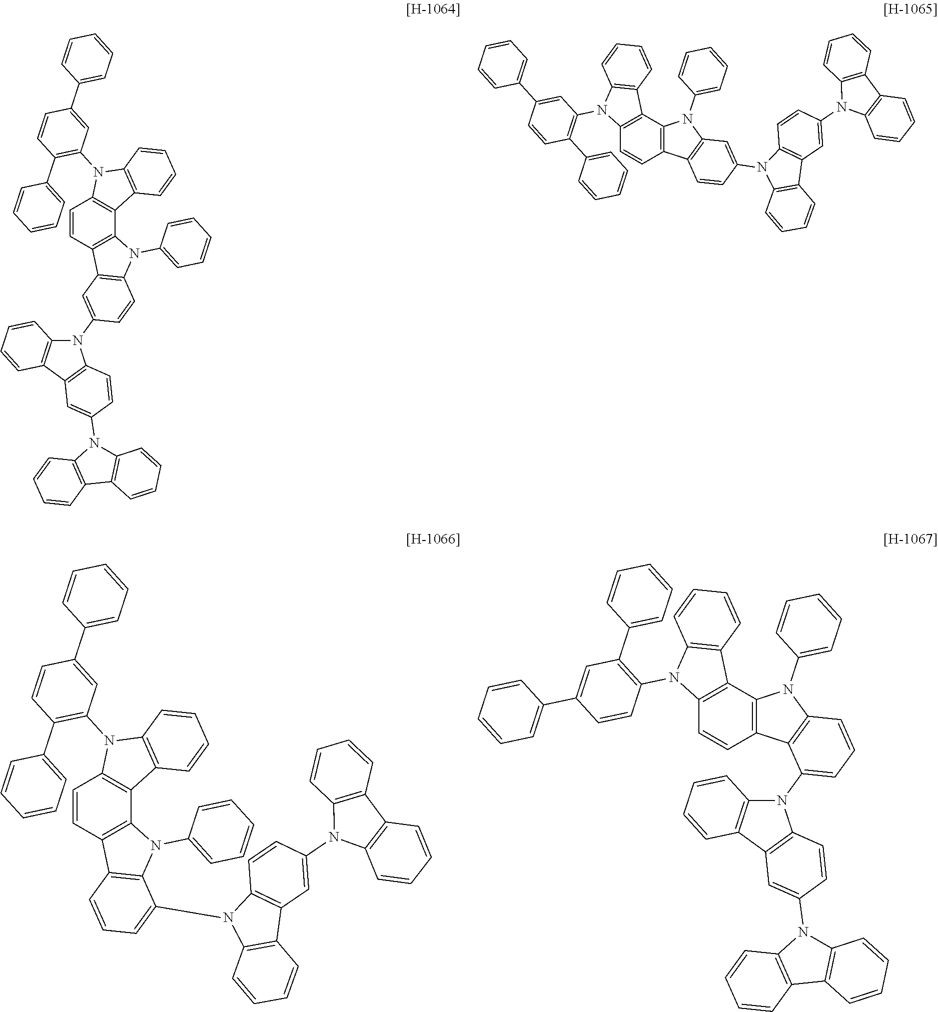 [SMILES: c1ccc(-c2ccc(-c3ccccc3)c(-n3c4ccccc4c4c3ccc3c5cc(-n6c7ccccc7c7cc(-n8c9ccccc9c9ccccc98)ccc76)ccc5n(-c5ccccc5)c34)c2)cc1 ; c1ccc(-c2ccc(-c3ccccc3)c(-n3c4ccccc4c4c3ccc3c5ccc(-n6c7ccccc7c7cc(-n8c9ccccc9c9ccccc98)ccc76)cc5n(-c5ccccc5)c34)c2)cc1 ; c1ccc(-c2ccc(-c3ccccc3)c(-n3c4ccccc4c4c3ccc3c5cccc(-n6c7ccccc7c7cc(-n8c9ccccc9c9ccccc98)ccc76)c5n(-c5ccccc5)c34)c2)cc1 ; c1ccc(-c2ccc(-n3c4ccccc4c4c3ccc3c5c(-n6c7ccccc7c7cc(-n8c9ccccc9c9ccccc98)ccc76)cccc5n(-c5ccccc5)c34)c(-c3ccccc3)c2)cc1]

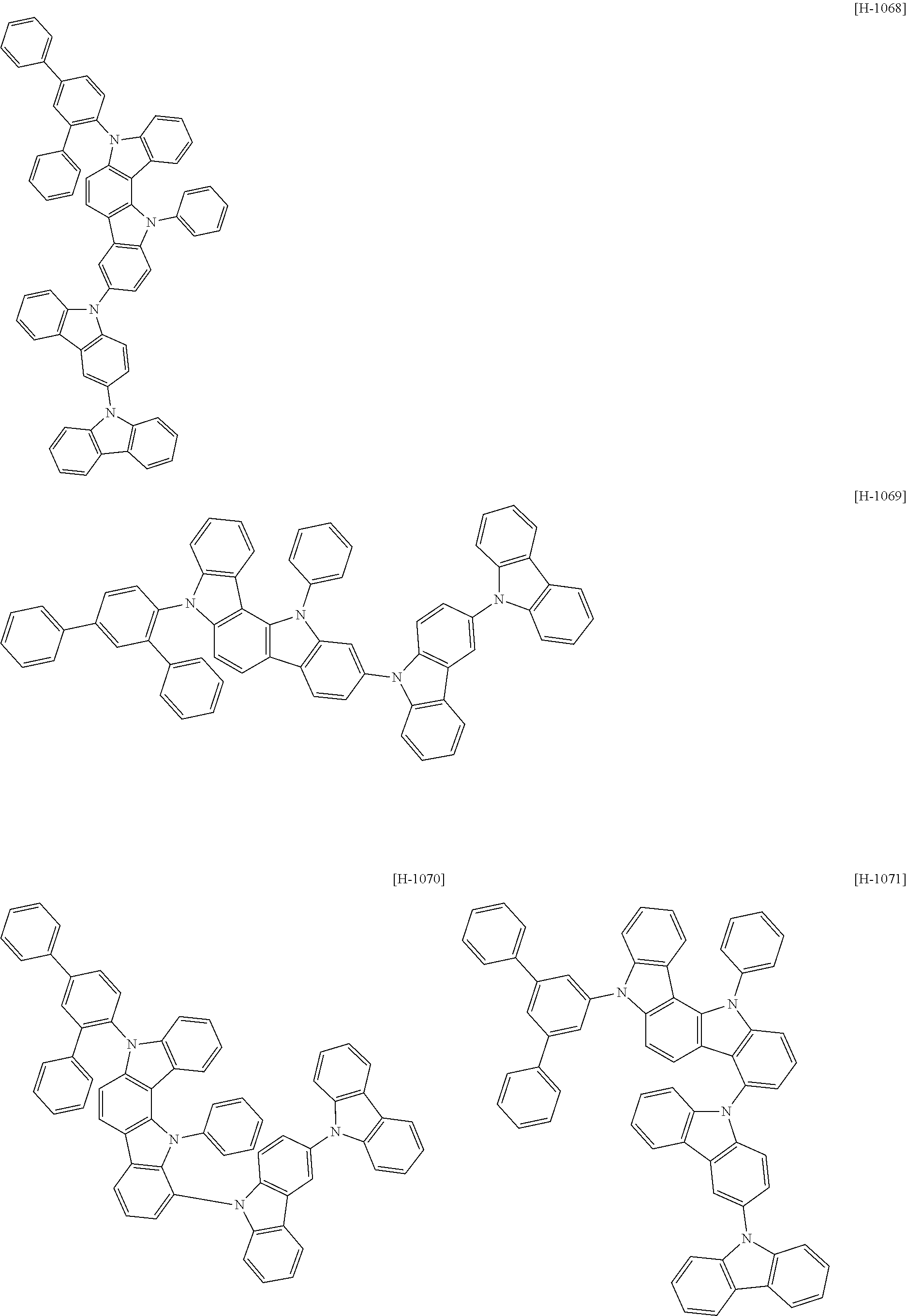 [SMILES: c1ccc(-c2ccc(-n3c4ccccc4c4c3ccc3c5cc(-n6c7ccccc7c7cc(-n8c9ccccc9c9ccccc98)ccc76)ccc5n(-c5ccccc5)c34)c(-c3ccccc3)c2)cc1 ; c1ccc(-c2ccc(-n3c4ccccc4c4c3ccc3c5ccc(-n6c7ccccc7c7cc(-n8c9ccccc9c9ccccc98)ccc76)cc5n(-c5ccccc5)c34)c(-c3ccccc3)c2)cc1 ; c1ccc(-c2ccc(-n3c4ccccc4c4c3ccc3c5cccc(-n6c7ccccc7c7cc(-n8c9ccccc9c9ccccc98)ccc76)c5n(-c5ccccc5)c34)c(-c3ccccc3)c2)cc1 ; c1ccc(-c2cc(-c3ccccc3)cc(-n3c4ccccc4c4c3ccc3c5c(-n6c7ccccc7c7cc(-n8c9ccccc9c9ccccc98)ccc76)cccc5n(-c5ccccc5)c34)c2)cc1]

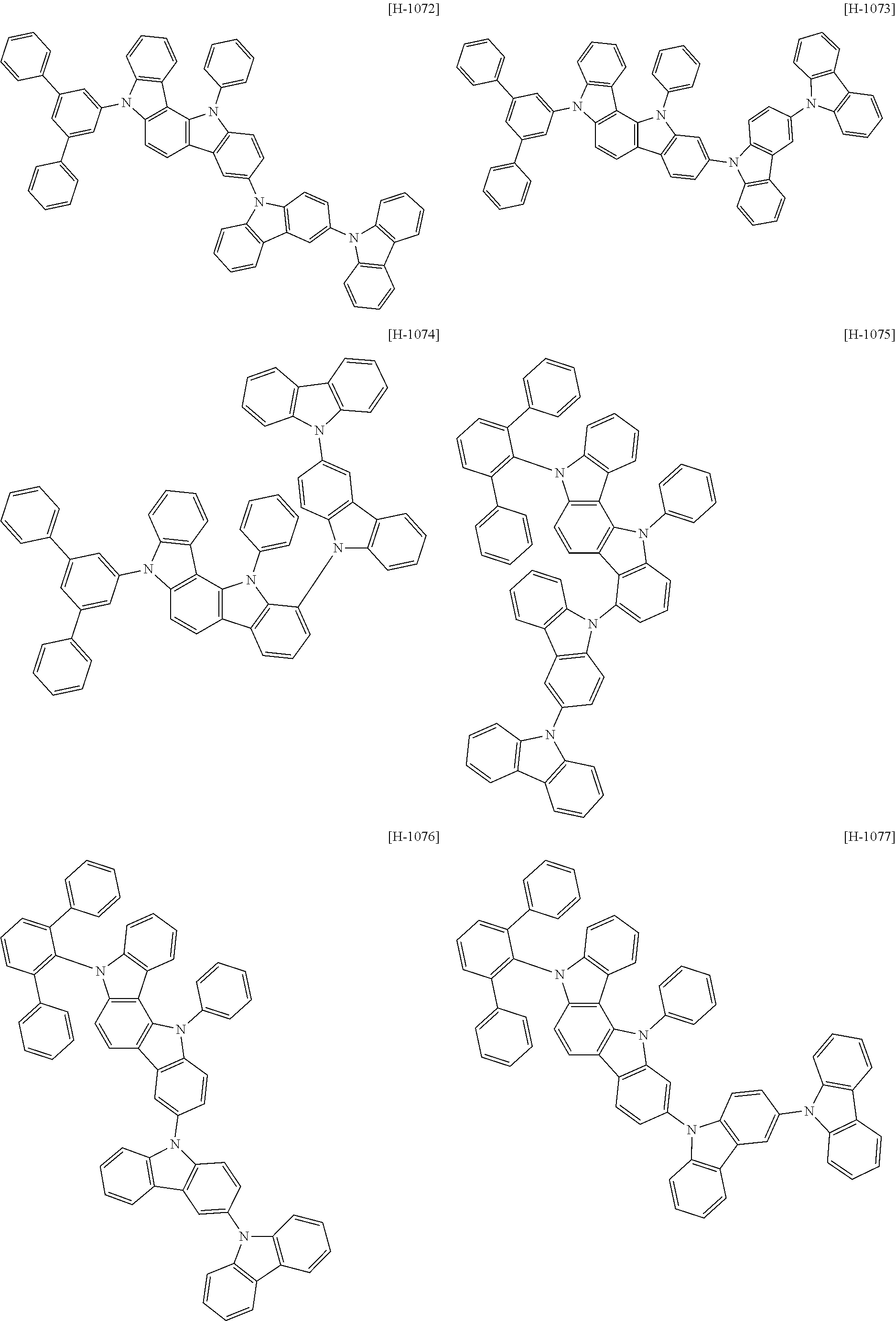

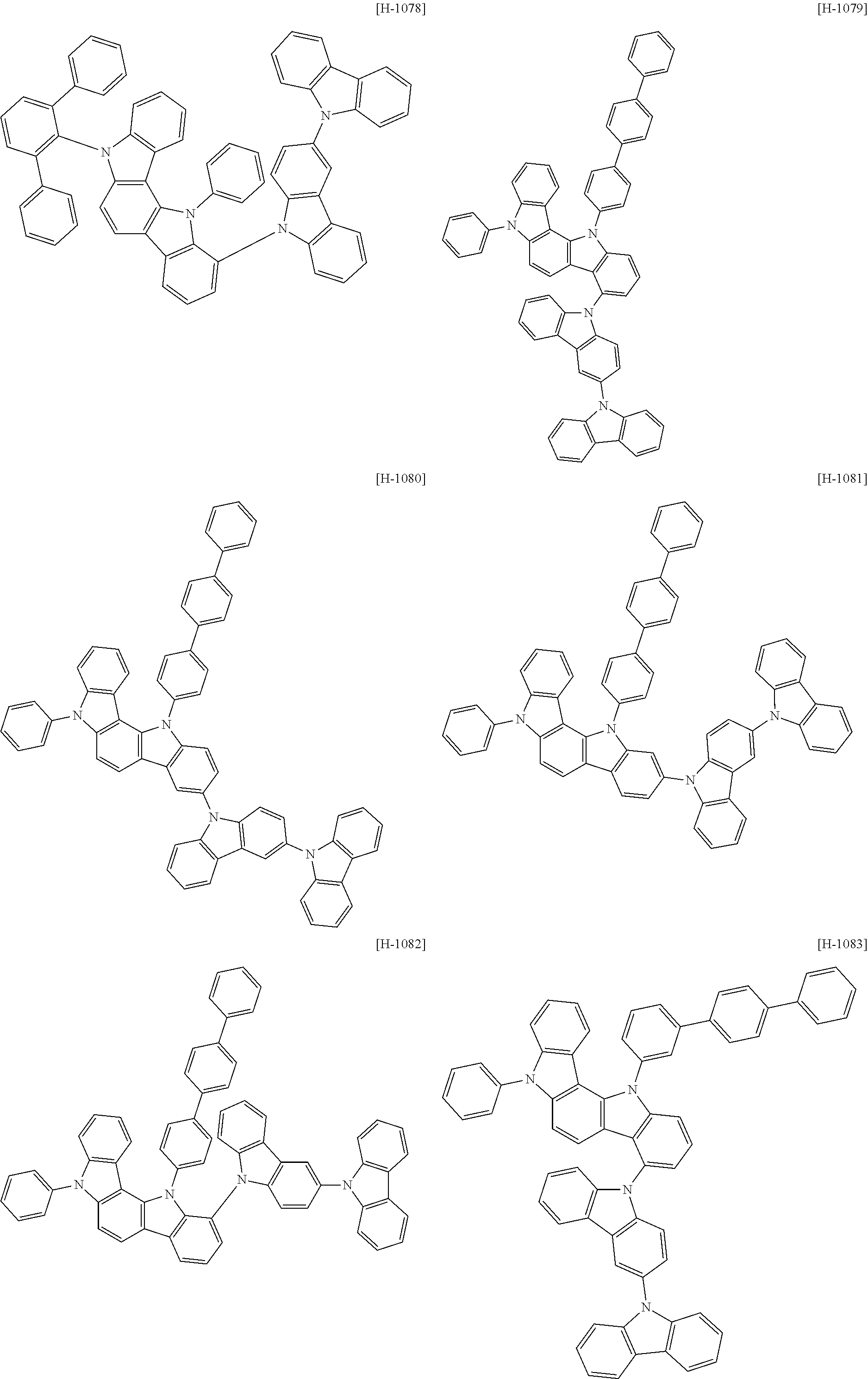

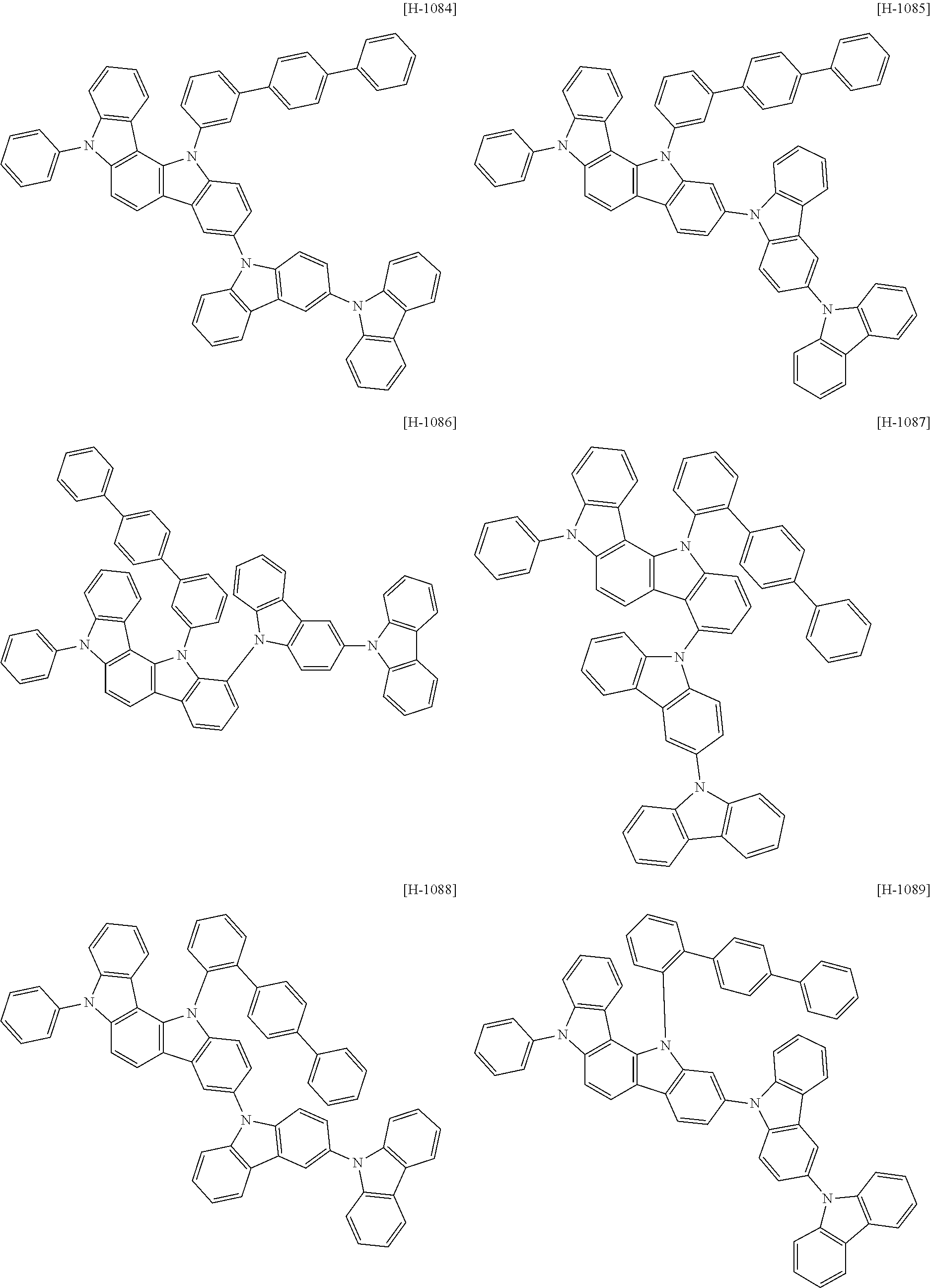

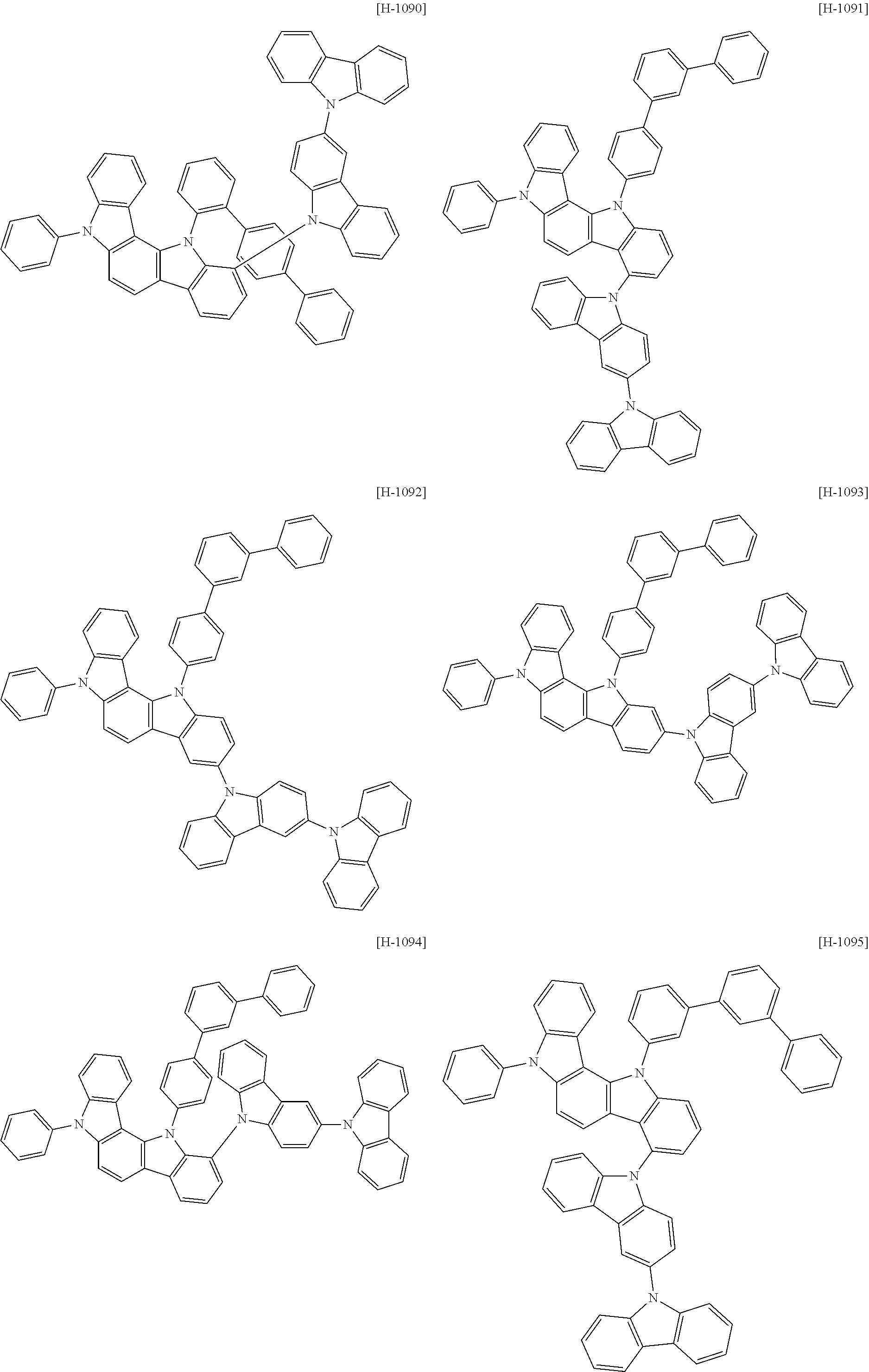

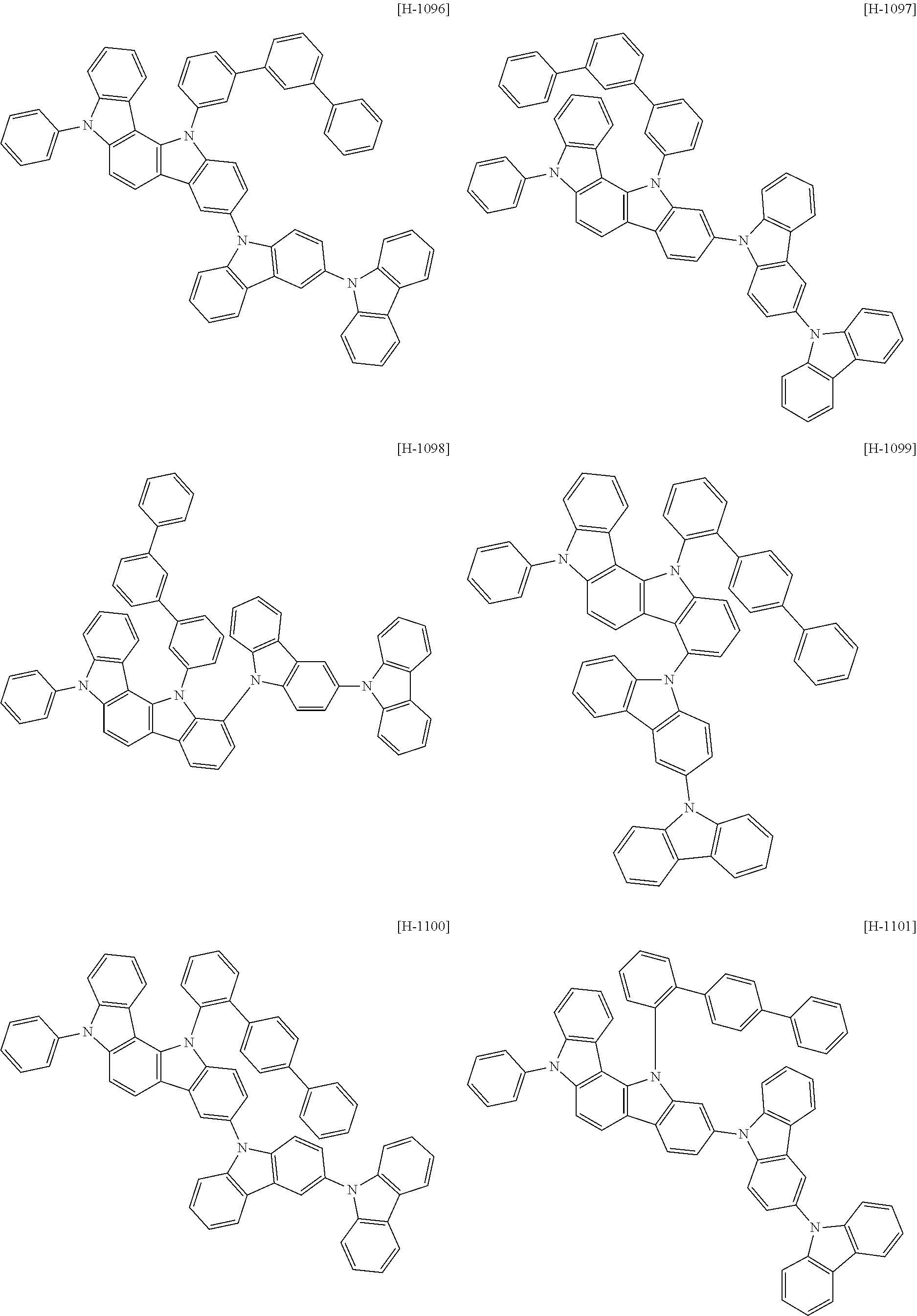

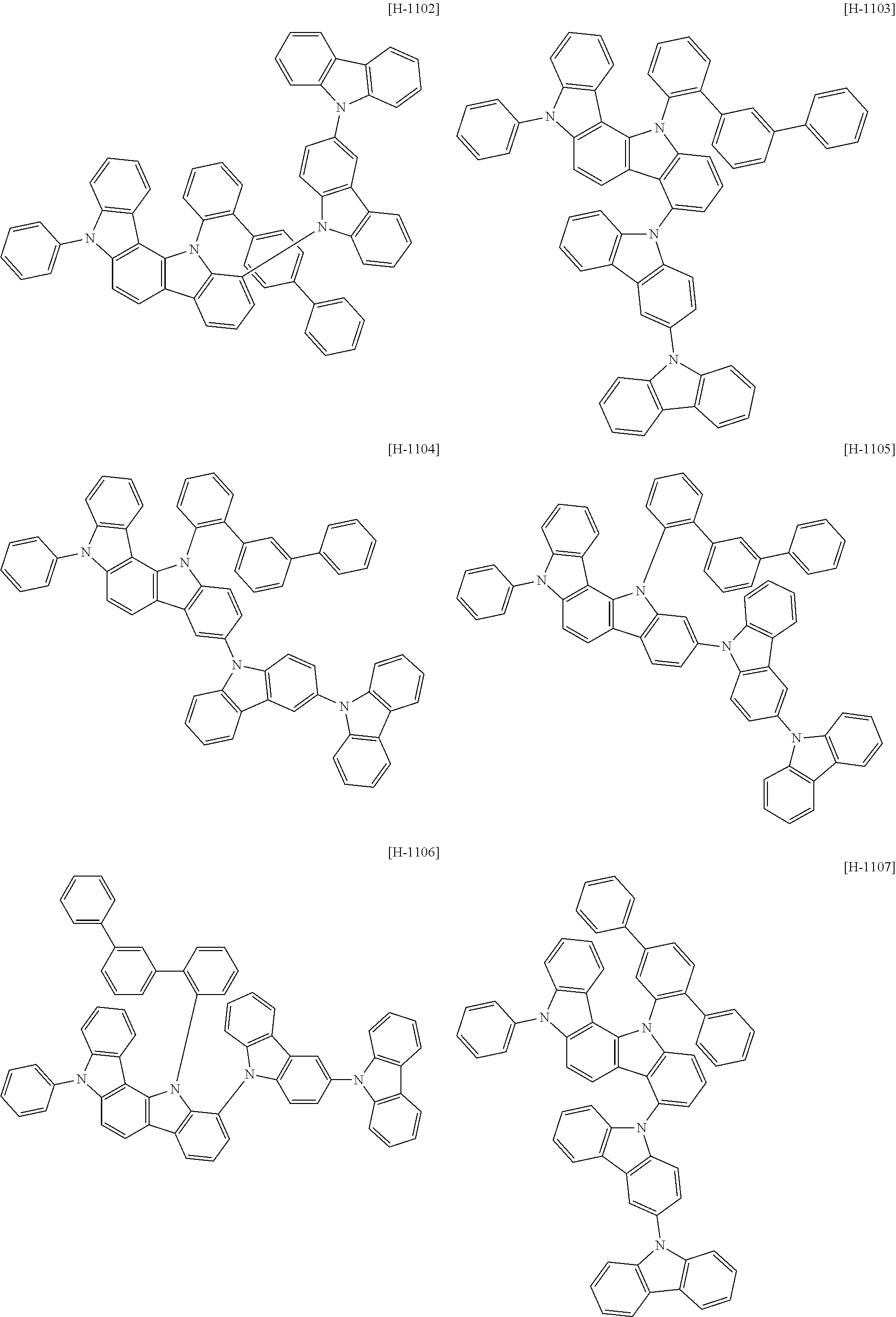

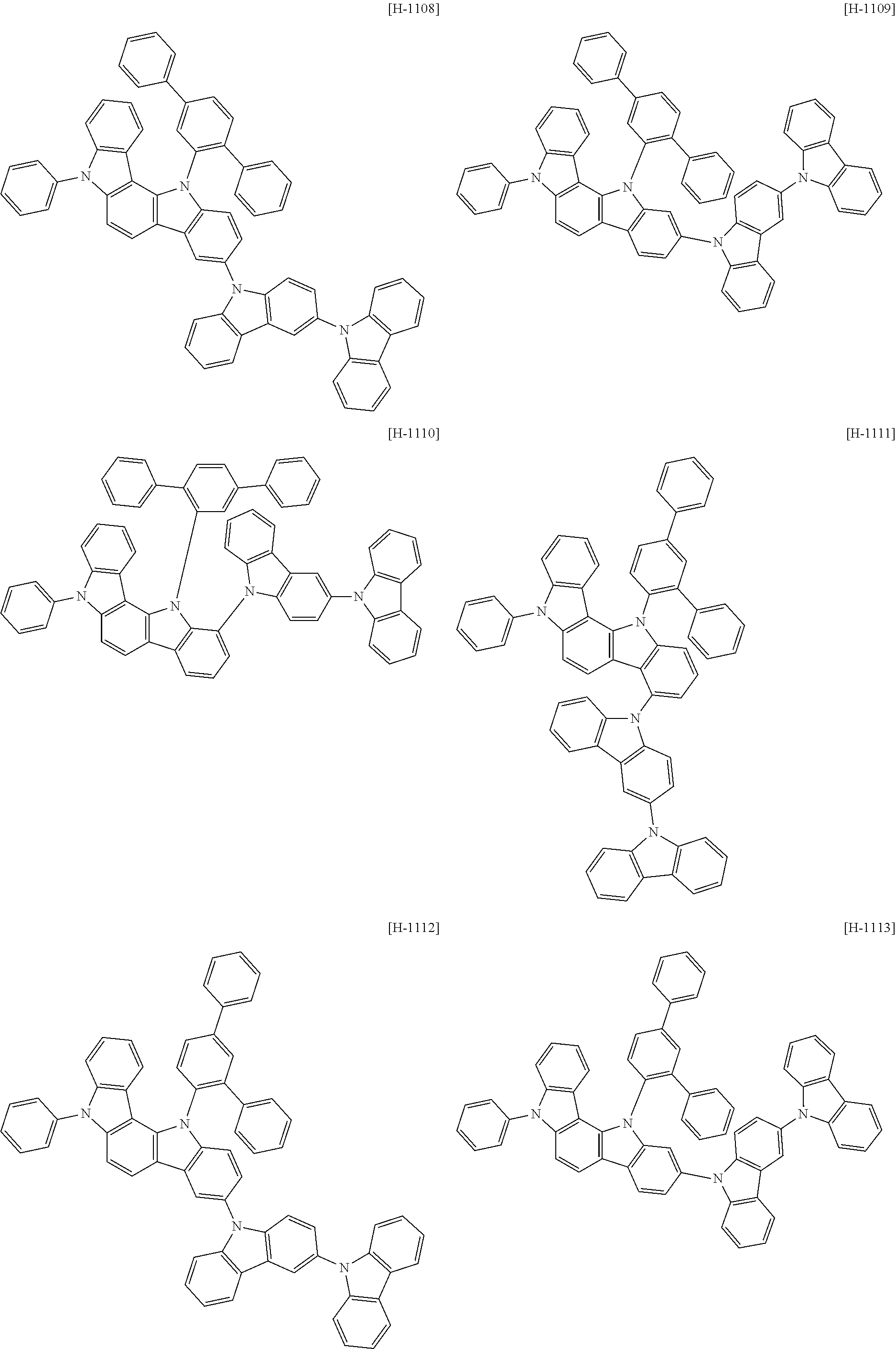

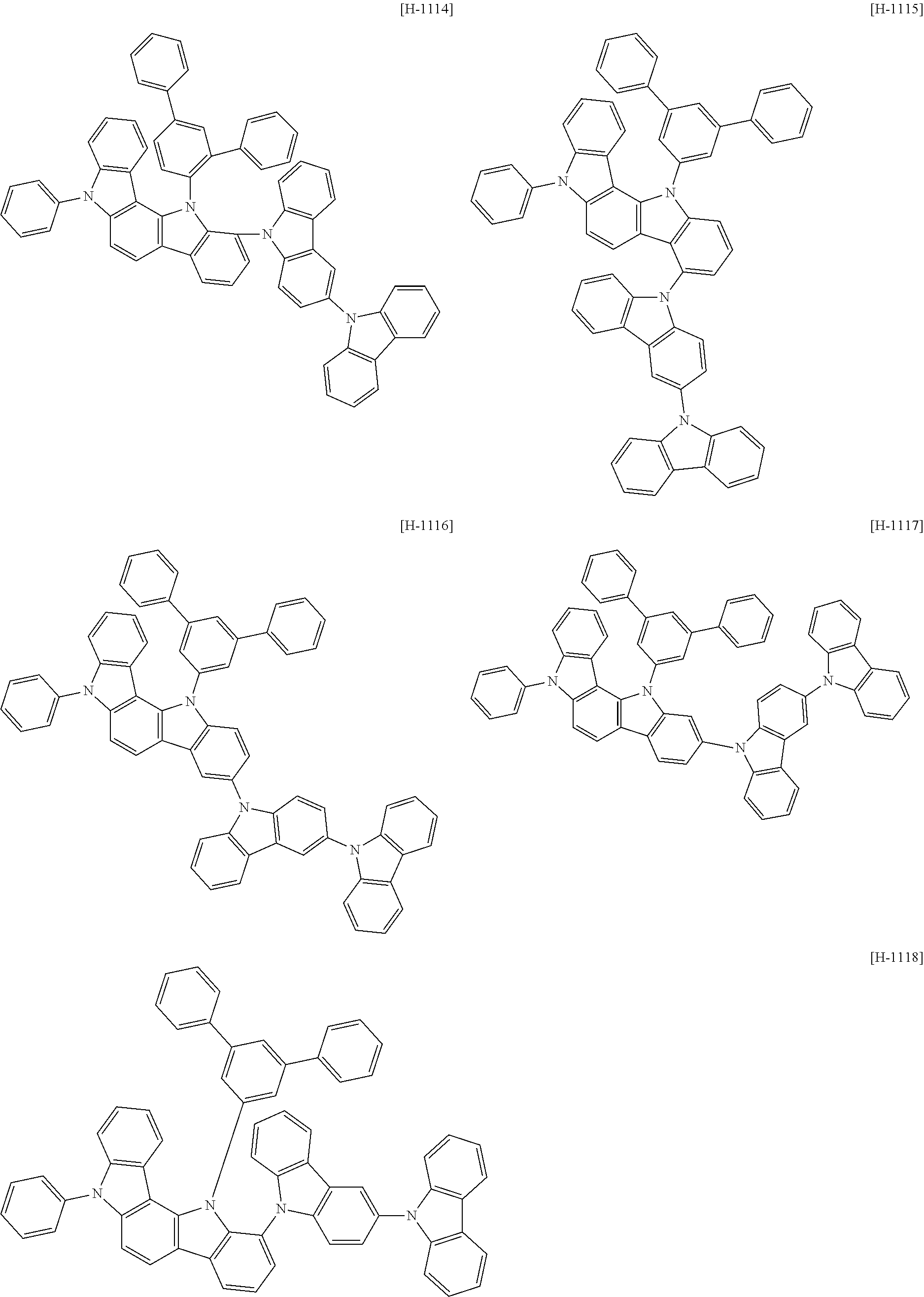

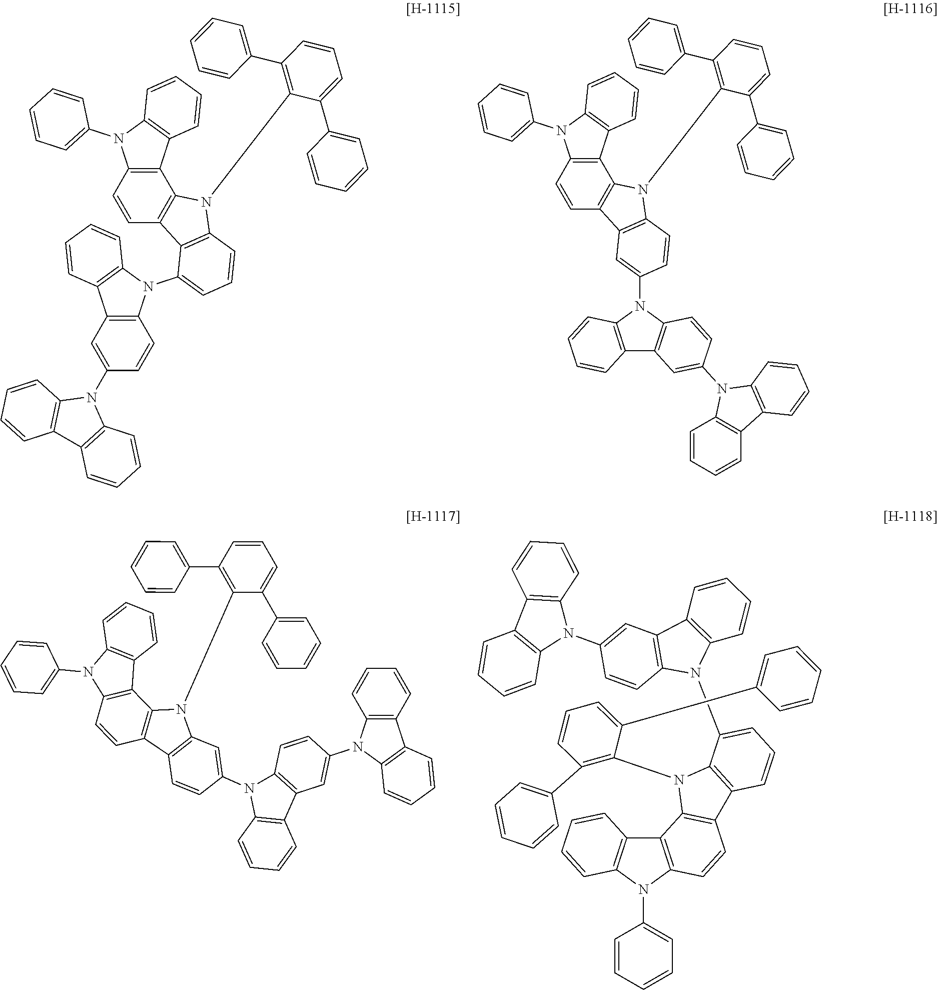 [SMILES: c1ccc(-c2cccc(-c3ccccc3)c2-n2c3cccc(-n4c5ccccc5c5cc(-n6c7ccccc7c7ccccc76)ccc54)c3c3ccc4c(c5ccccc5n4-c4ccccc4)c32)cc1 ; c1ccc(-c2cccc(-c3ccccc3)c2-n2c3ccc(-n4c5ccccc5c5cc(-n6c7ccccc7c7ccccc76)ccc54)cc3c3ccc4c(c5ccccc5n4-c4ccccc4)c32)cc1 ; c1ccc(-c2cccc(-c3ccccc3)c2-n2c3cc(-n4c5ccccc5c5cc(-n6c7ccccc7c7ccccc76)ccc54)ccc3c3ccc4c(c5ccccc5n4-c4ccccc4)c32)cc1 ; c1ccc(-c2cccc(-c3ccccc3)c2-n2c3c(-n4c5ccccc5c5cc(-n6c7ccccc7c7ccccc76)ccc54)cccc3c3ccc4c(c5ccccc5n4-c4ccccc4)c32)cc1]

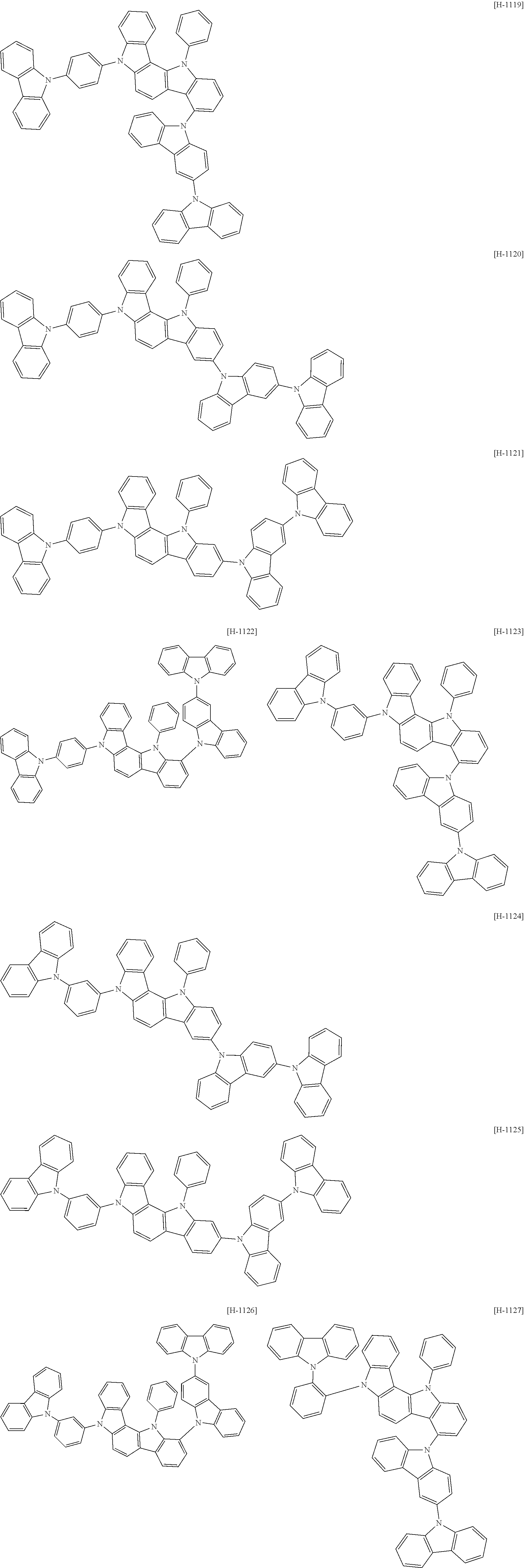

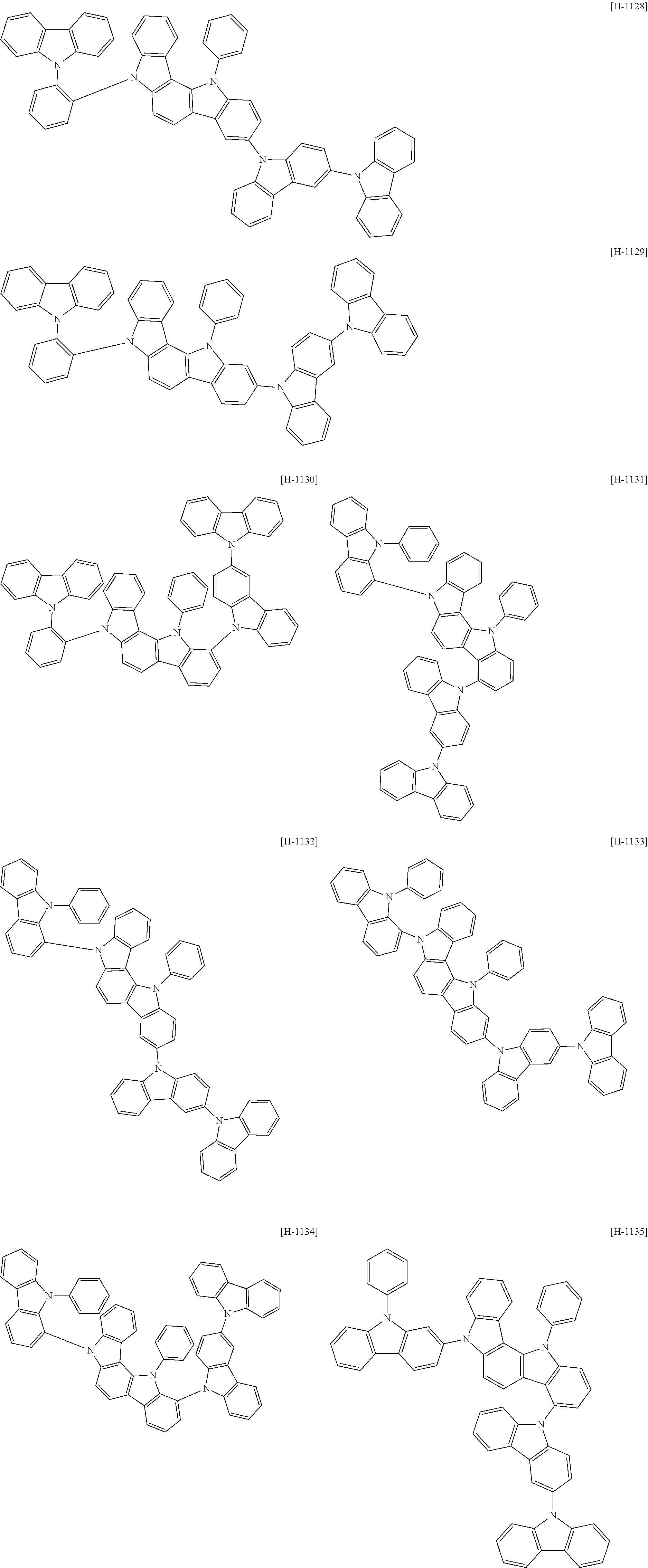

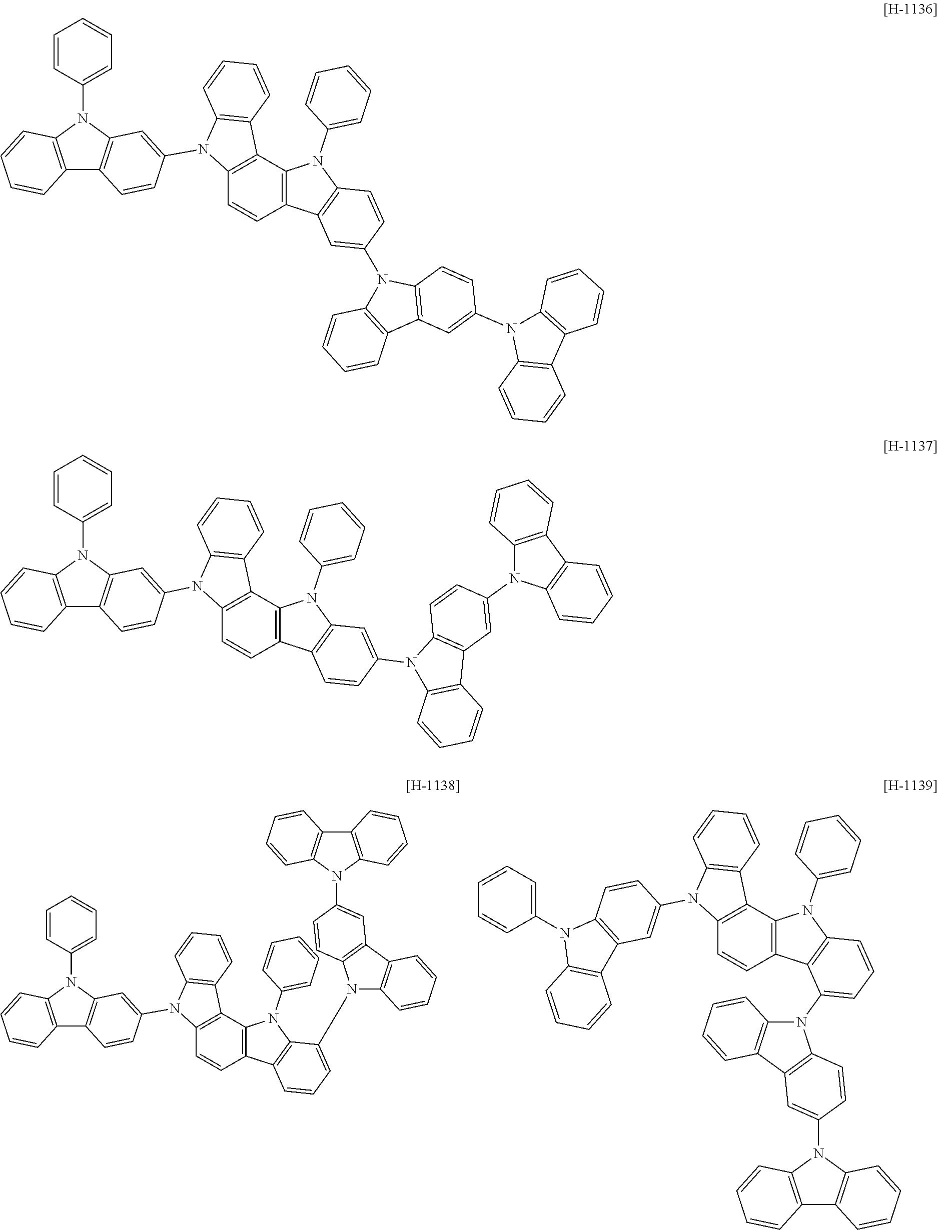

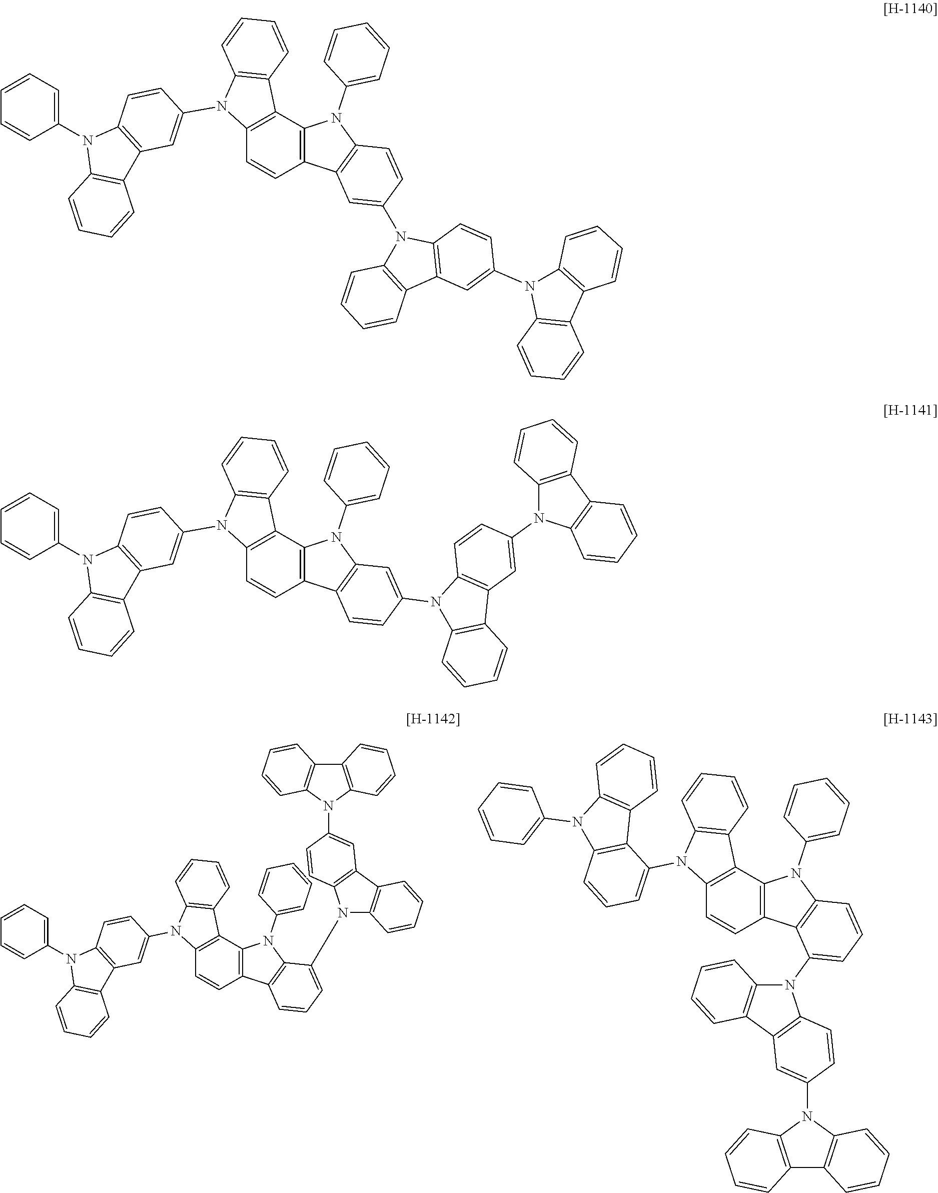

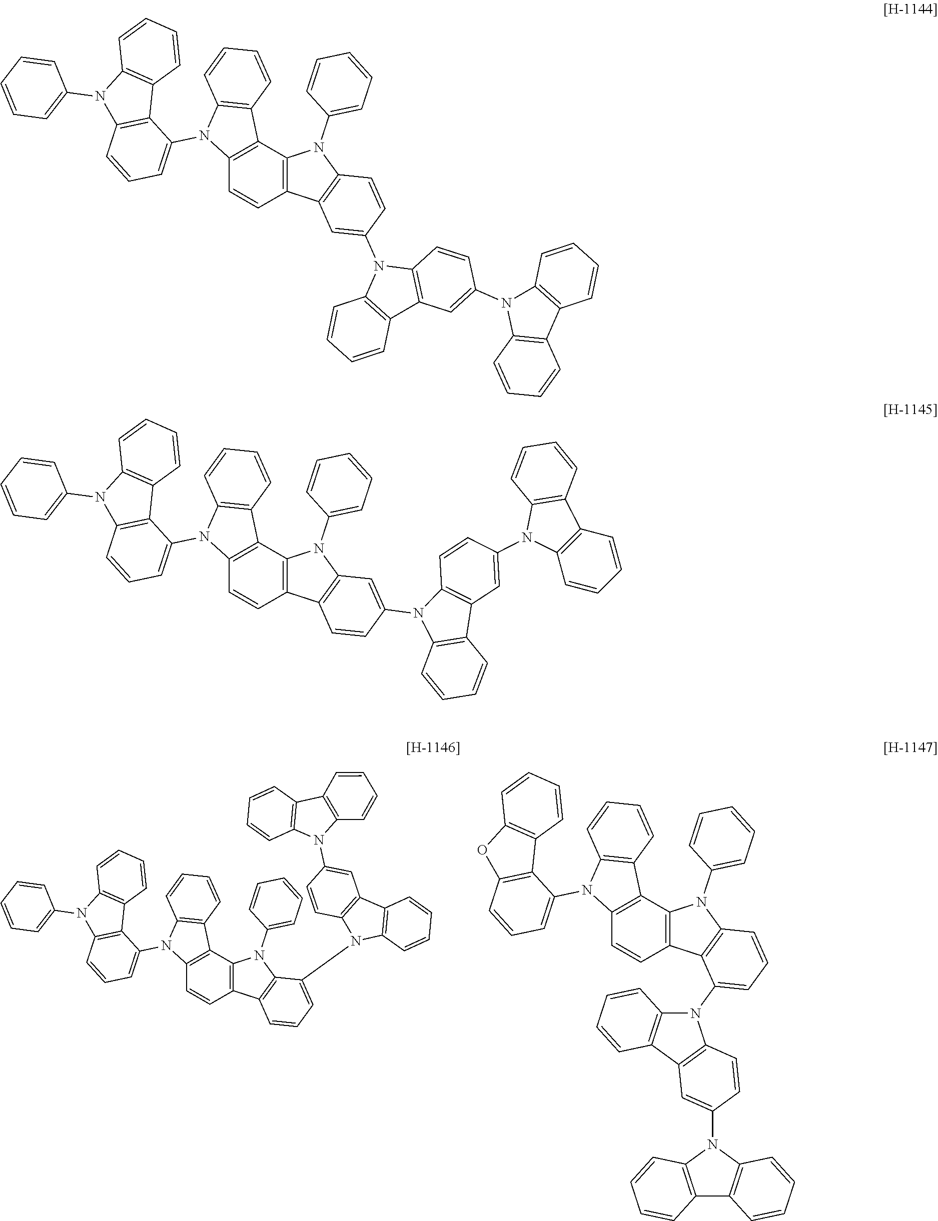 [SMILES: c1ccc(-n2c3ccccc3c3c(-n4c5ccccc5c5c4ccc4c6cc(-n7c8ccccc8c8cc(-n9c%10ccccc%10c%10ccccc%109)ccc87)ccc6n(-c6ccccc6)c45)cccc32)cc1 ; c1ccc(-n2c3ccccc3c3c(-n4c5ccccc5c5c4ccc4c6ccc(-n7c8ccccc8c8cc(-n9c%10ccccc%10c%10ccccc%109)ccc87)cc6n(-c6ccccc6)c45)cccc32)cc1 ; c1ccc(-n2c3ccccc3c3c(-n4c5ccccc5c5c4ccc4c6cccc(-n7c8ccccc8c8cc(-n9c%10ccccc%10c%10ccccc%109)ccc87)c6n(-c6ccccc6)c45)cccc32)cc1 ; c1ccc(-n2c3cccc(-n4c5ccccc5c5cc(-n6c7ccccc7c7ccccc76)ccc54)c3c3ccc4c(c5ccccc5n4-c4cccc5oc6ccccc6c45)c32)cc1]

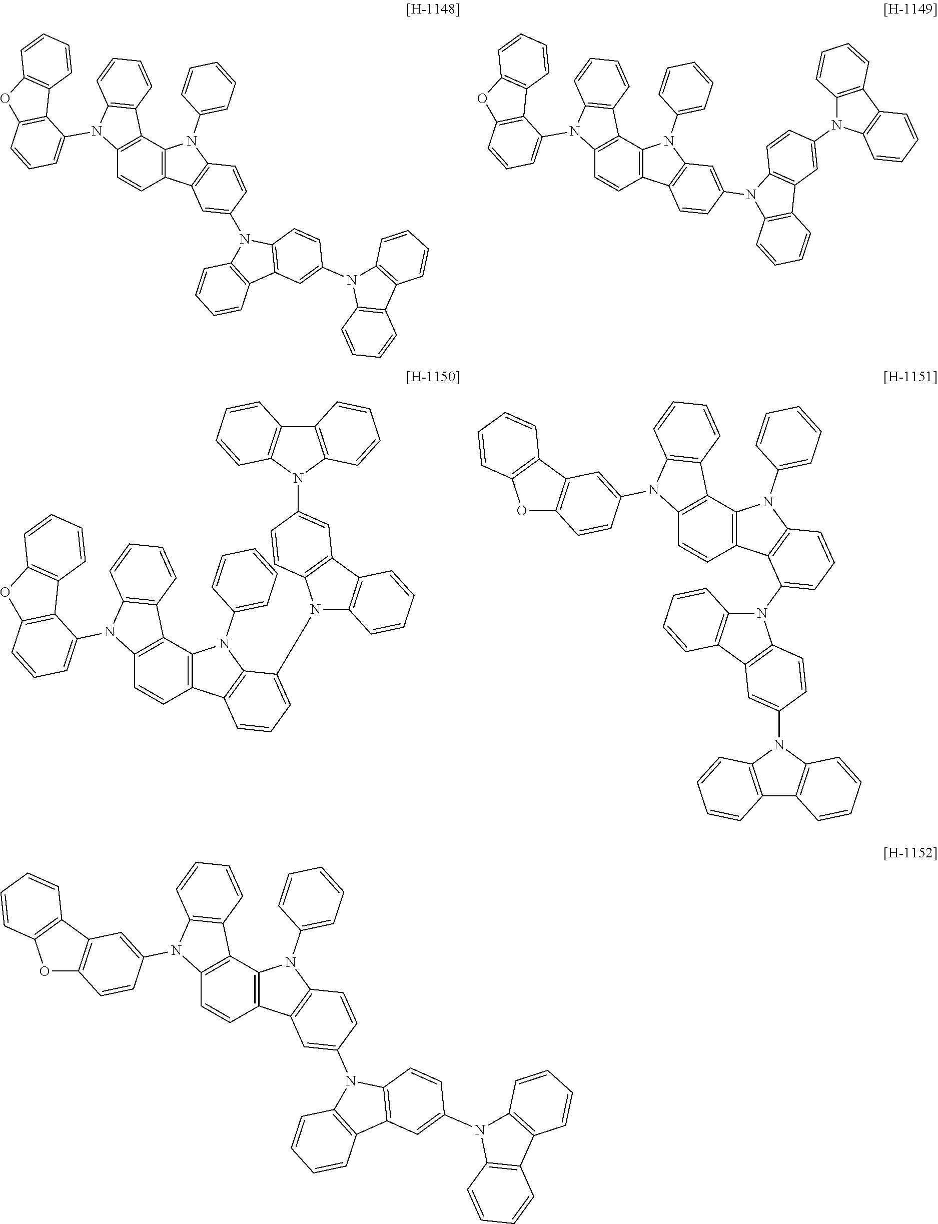

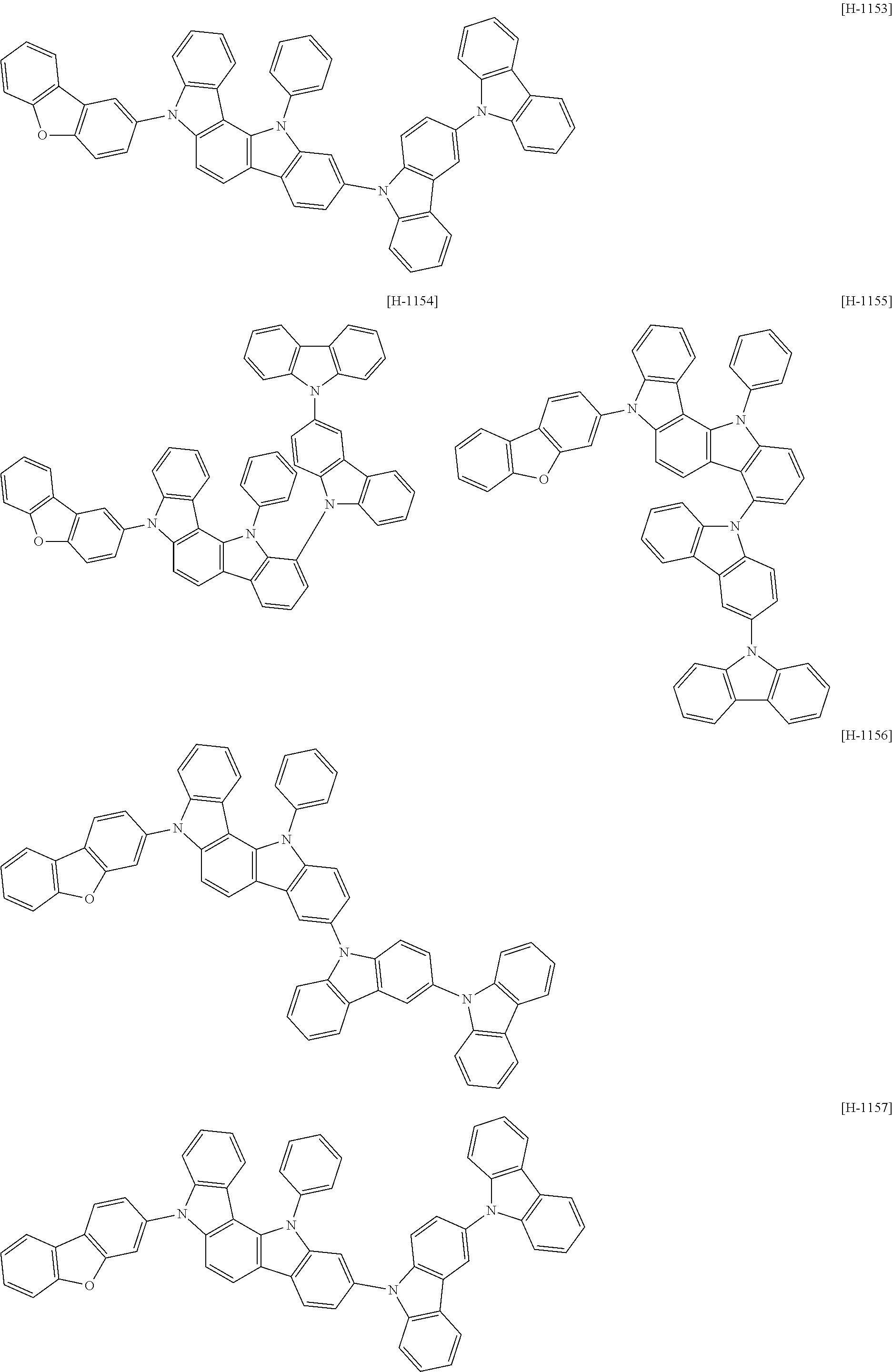

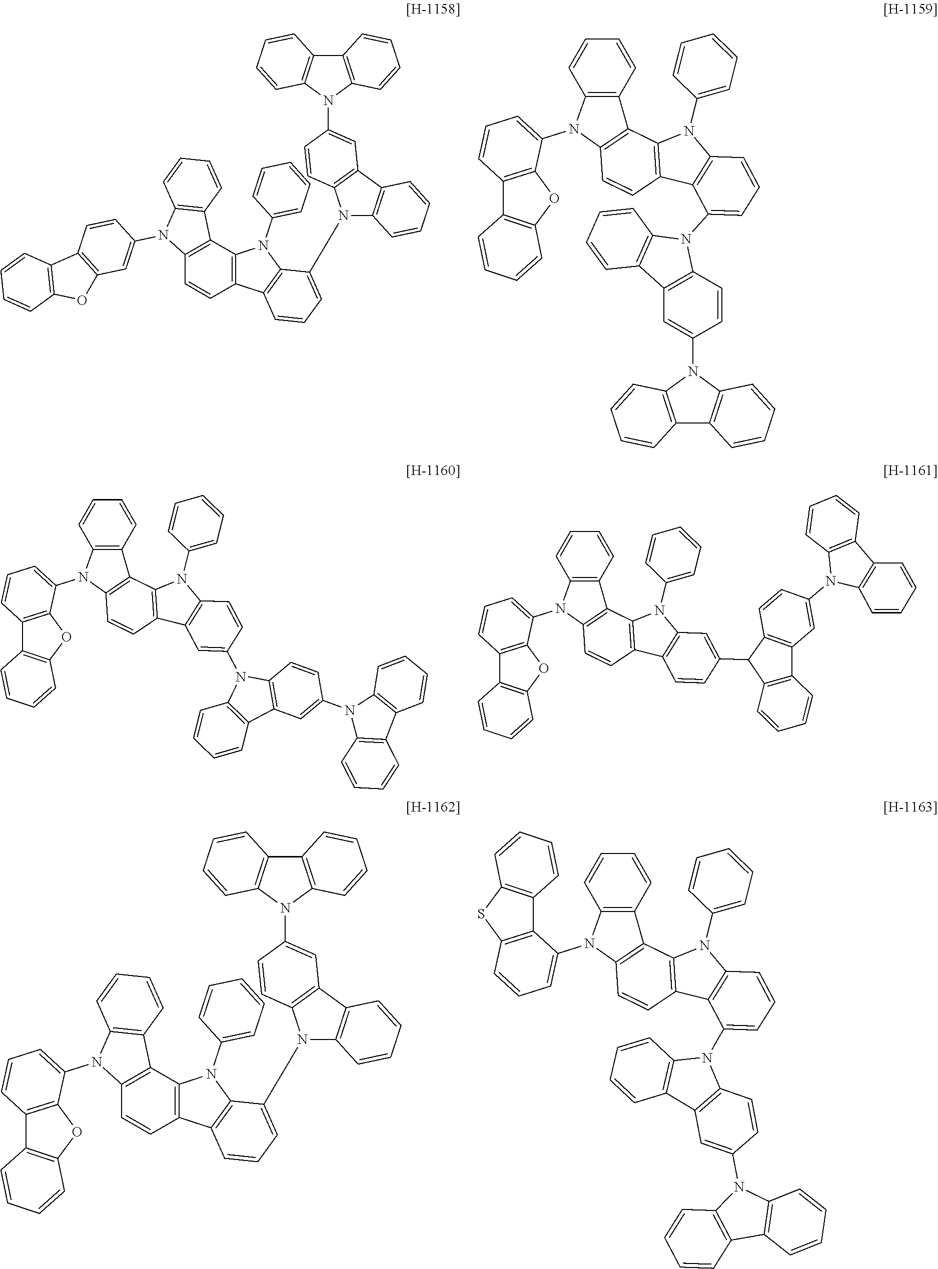

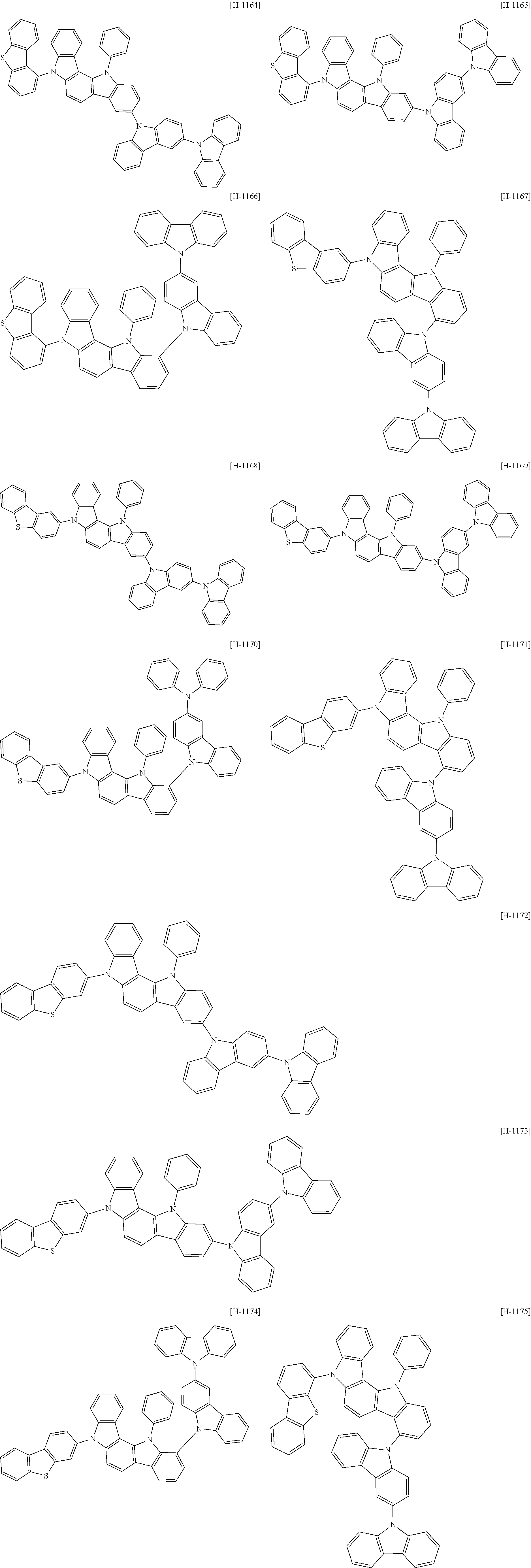

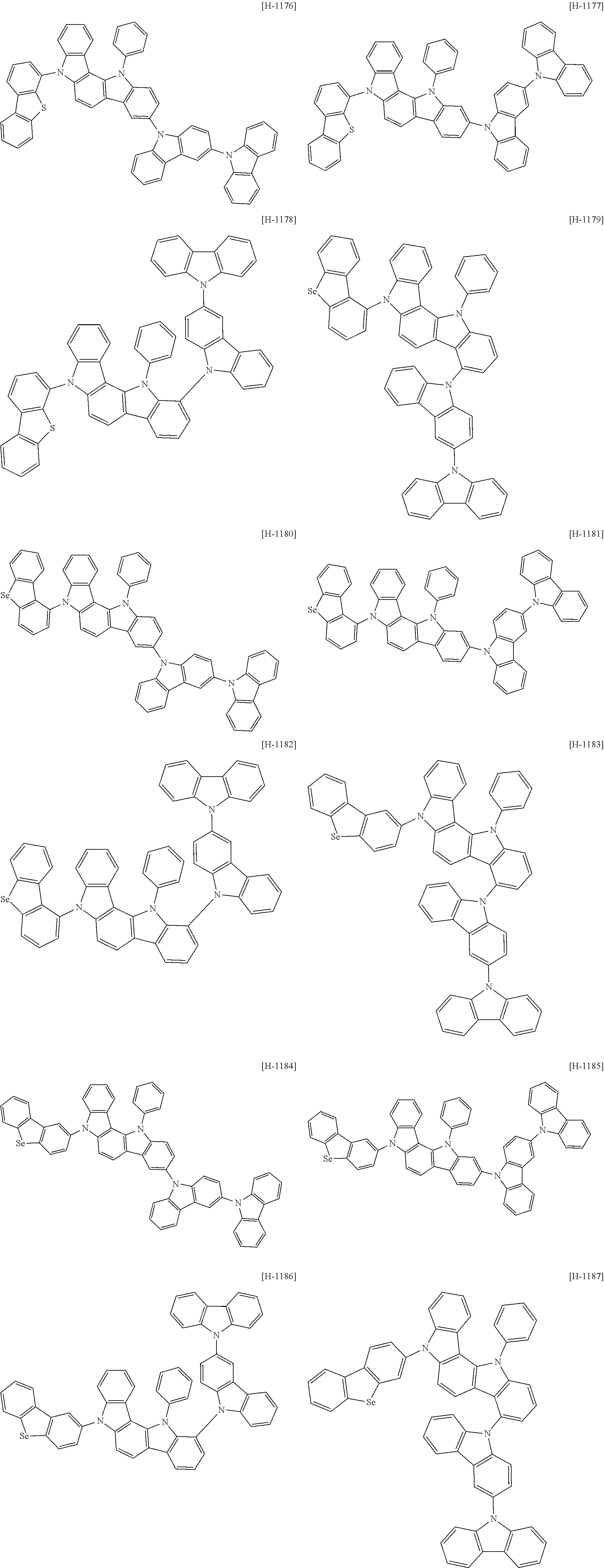

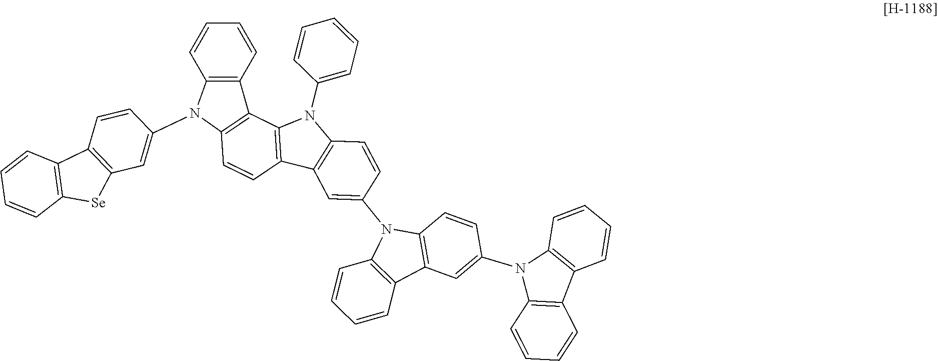 [SMILES: c1ccc(-n2c3ccc(-n4c5ccccc5c5cc(-n6c7ccccc7c7ccccc76)ccc54)cc3c3ccc4c(c5ccccc5n4-c4ccc5c(c4)[se]c4ccccc45)c32)cc1]

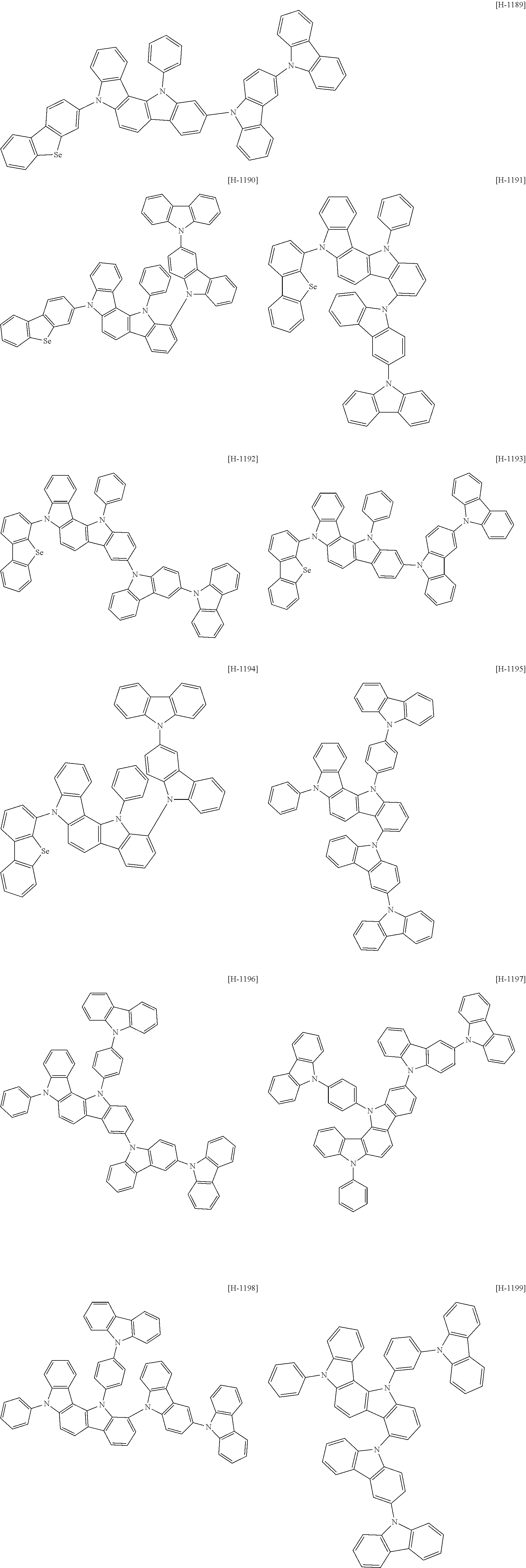

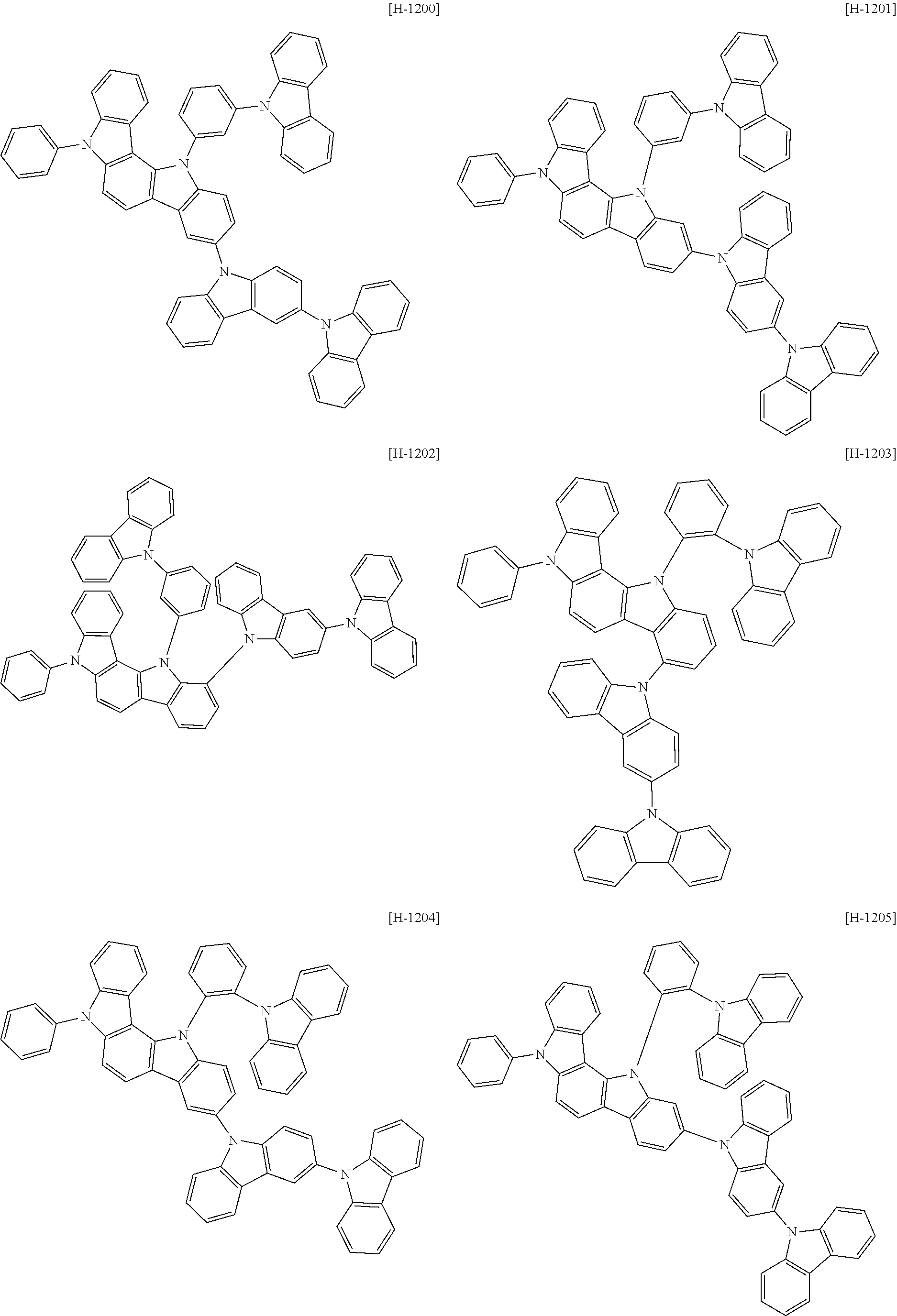

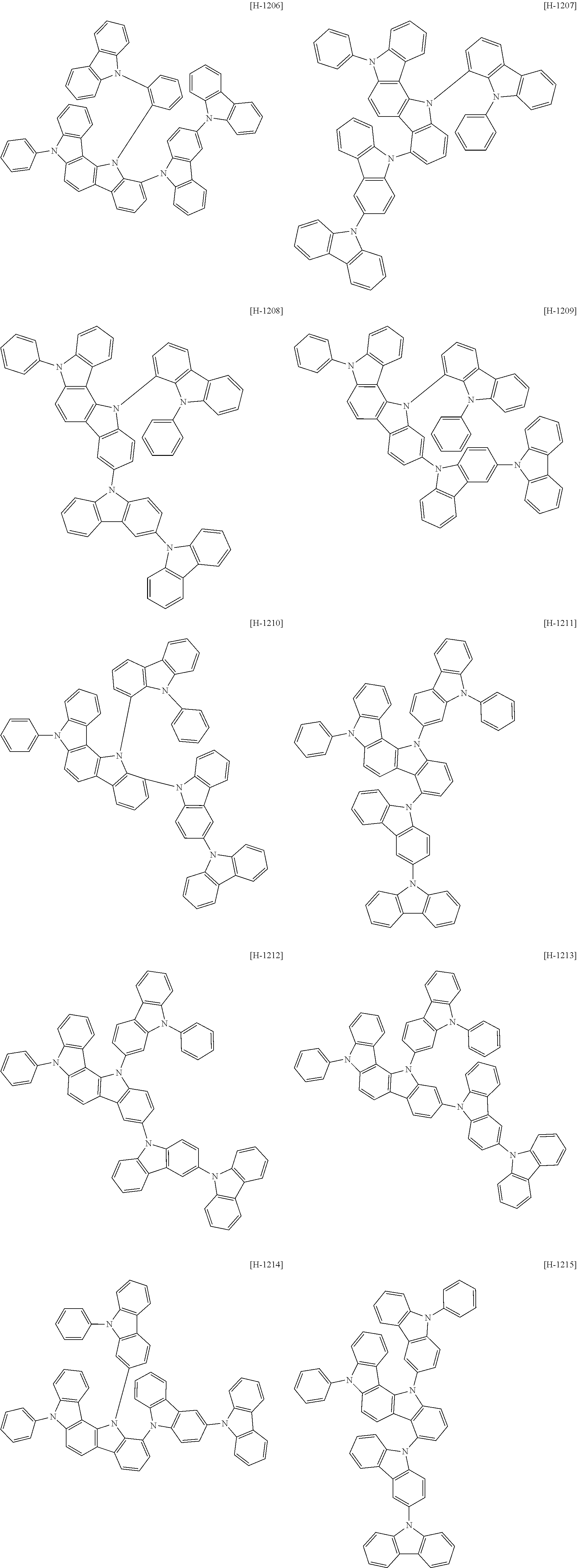

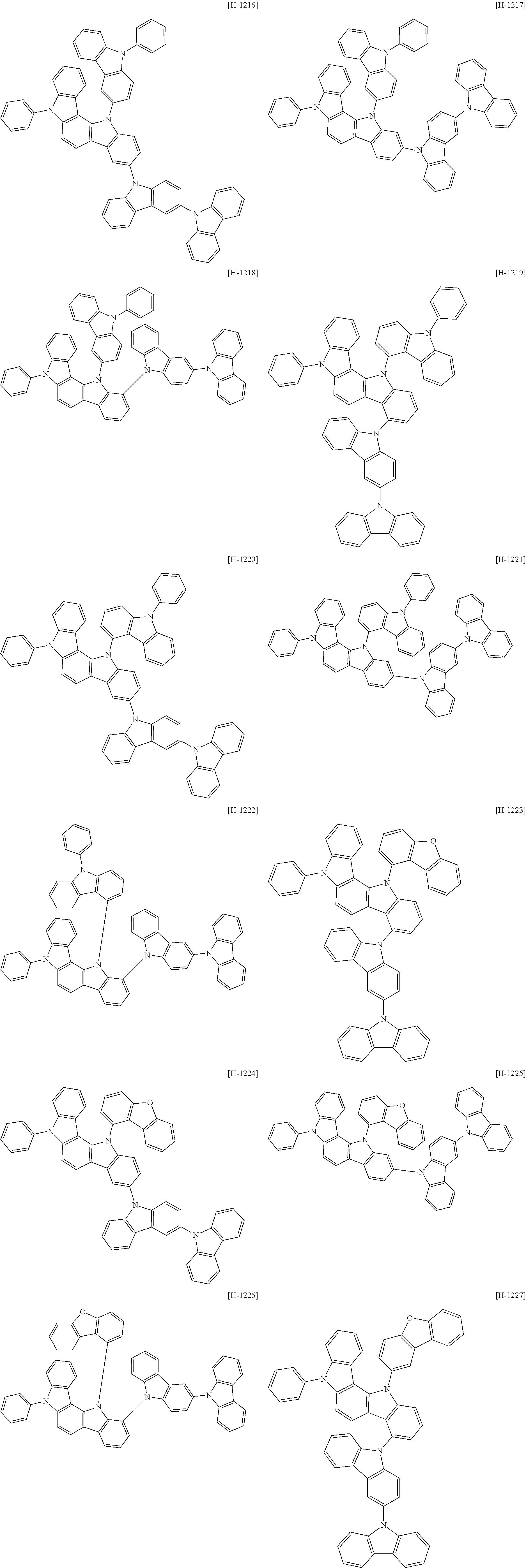

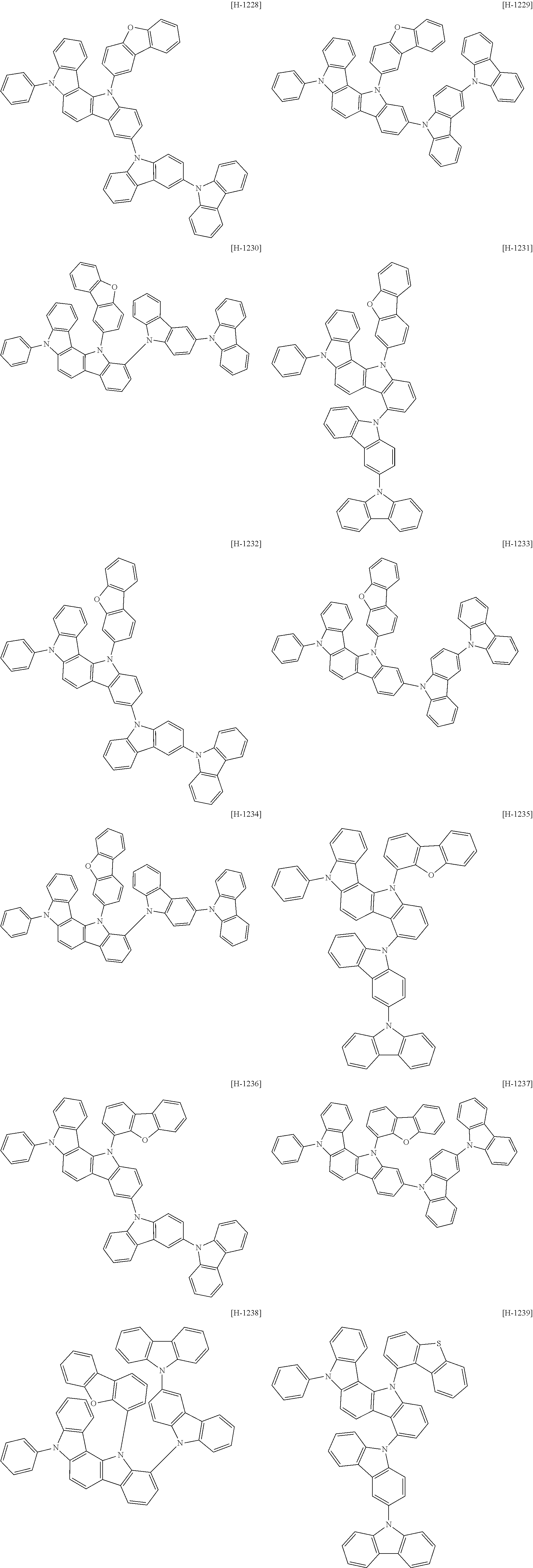

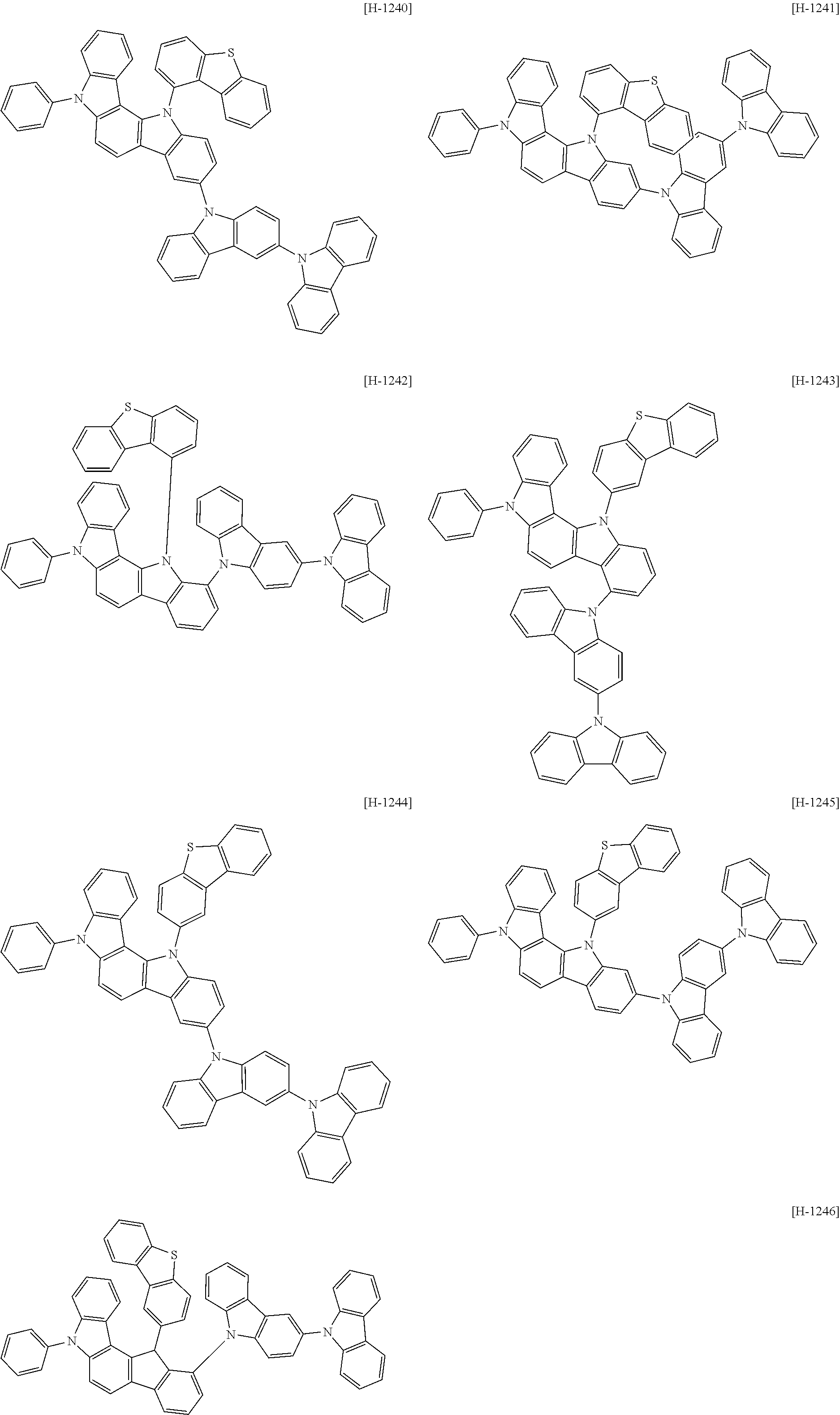

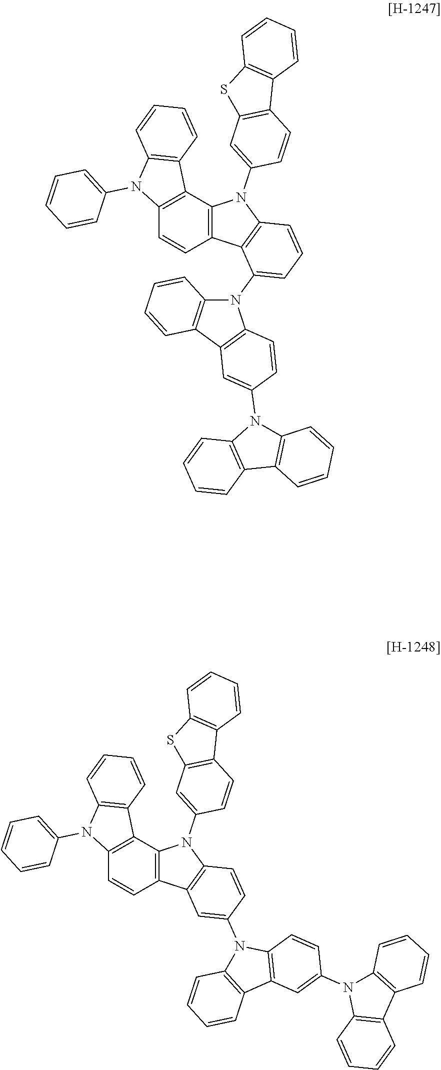 [SMILES: c1ccc(-n2c3ccccc3c3c2ccc2c4c(-n5c6ccccc6c6cc(-n7c8ccccc8c8ccccc87)ccc65)cccc4n(-c4ccc5c(c4)sc4ccccc45)c23)cc1 ; c1ccc(-n2c3ccccc3c3c2ccc2c4cc(-n5c6ccccc6c6cc(-n7c8ccccc8c8ccccc87)ccc65)ccc4n(-c4ccc5c(c4)sc4ccccc45)c23)cc1]

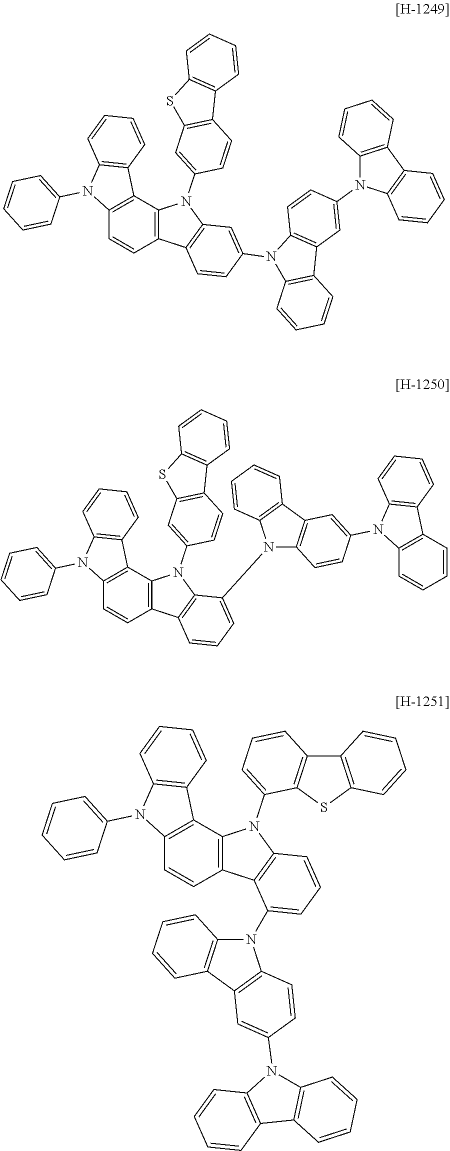 [SMILES: c1ccc(-n2c3ccccc3c3c2ccc2c4ccc(-n5c6ccccc6c6cc(-n7c8ccccc8c8ccccc87)ccc65)cc4n(-c4ccc5c(c4)sc4ccccc45)c23)cc1 ; c1ccc(-n2c3ccccc3c3c2ccc2c4cccc(-n5c6ccccc6c6cc(-n7c8ccccc8c8ccccc87)ccc65)c4n(-c4ccc5c(c4)sc4ccccc45)c23)cc1 ; c1ccc(-n2c3ccccc3c3c2ccc2c4c(-n5c6ccccc6c6cc(-n7c8ccccc8c8ccccc87)ccc65)cccc4n(-c4cccc5c4sc4ccccc45)c23)cc1]

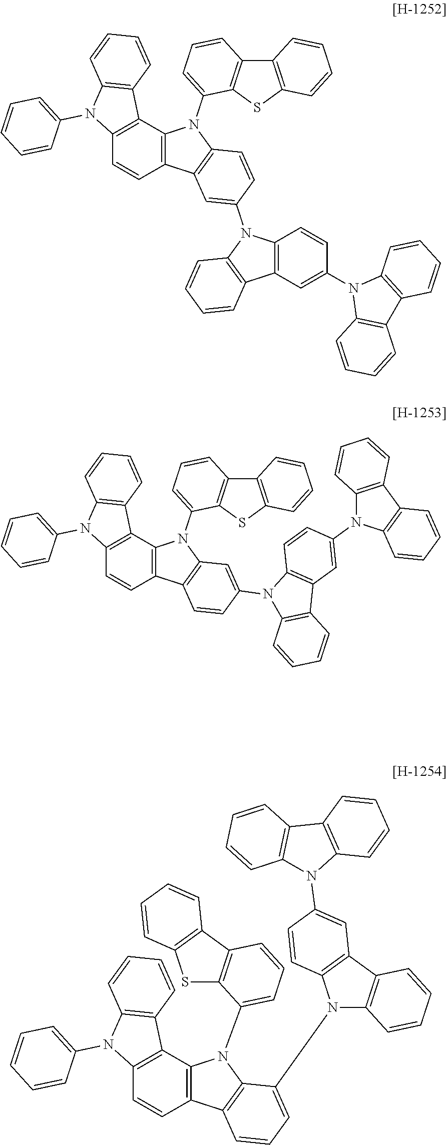 [SMILES: c1ccc(-n2c3ccccc3c3c2ccc2c4cc(-n5c6ccccc6c6cc(-n7c8ccccc8c8ccccc87)ccc65)ccc4n(-c4cccc5c4sc4ccccc45)c23)cc1 ; c1ccc(-n2c3ccccc3c3c2ccc2c4ccc(-n5c6ccccc6c6cc(-n7c8ccccc8c8ccccc87)ccc65)cc4n(-c4cccc5c4sc4ccccc45)c23)cc1 ; c1ccc(-n2c3ccccc3c3c2ccc2c4cccc(-n5c6ccccc6c6cc(-n7c8ccccc8c8ccccc87)ccc65)c4n(-c4cccc5c4sc4ccccc45)c23)cc1]

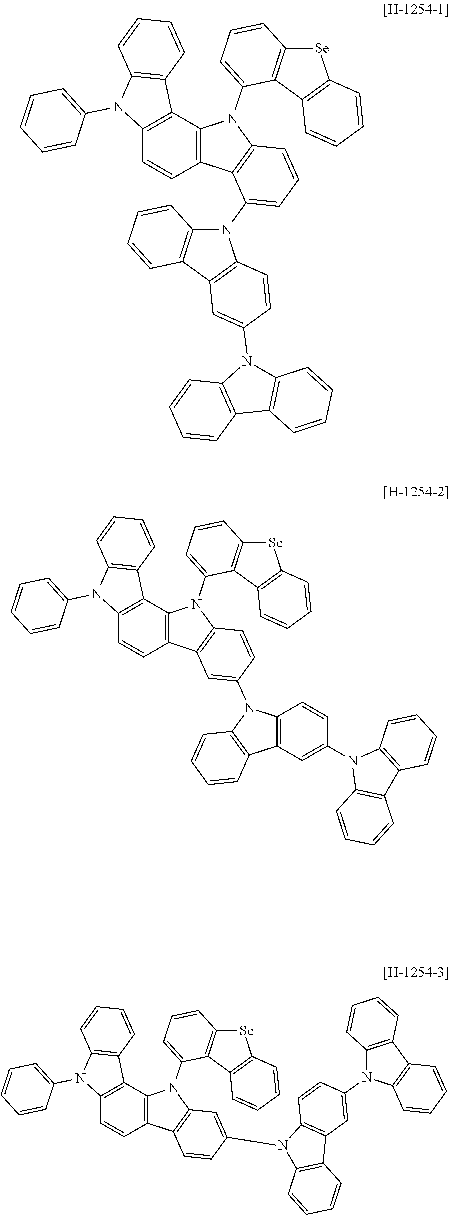 [SMILES: c1ccc(-n2c3ccccc3c3c2ccc2c4c(-n5c6ccccc6c6cc(-n7c8ccccc8c8ccccc87)ccc65)cccc4n(-c4cccc5[se]c6ccccc6c45)c23)cc1 ; c1ccc(-n2c3ccccc3c3c2ccc2c4cc(-n5c6ccccc6c6cc(-n7c8ccccc8c8ccccc87)ccc65)ccc4n(-c4cccc5[se]c6ccccc6c45)c23)cc1 ; c1ccc(-n2c3ccccc3c3c2ccc2c4ccc(-n5c6ccccc6c6cc(-n7c8ccccc8c8ccccc87)ccc65)cc4n(-c4cccc5[se]c6ccccc6c45)c23)cc1]

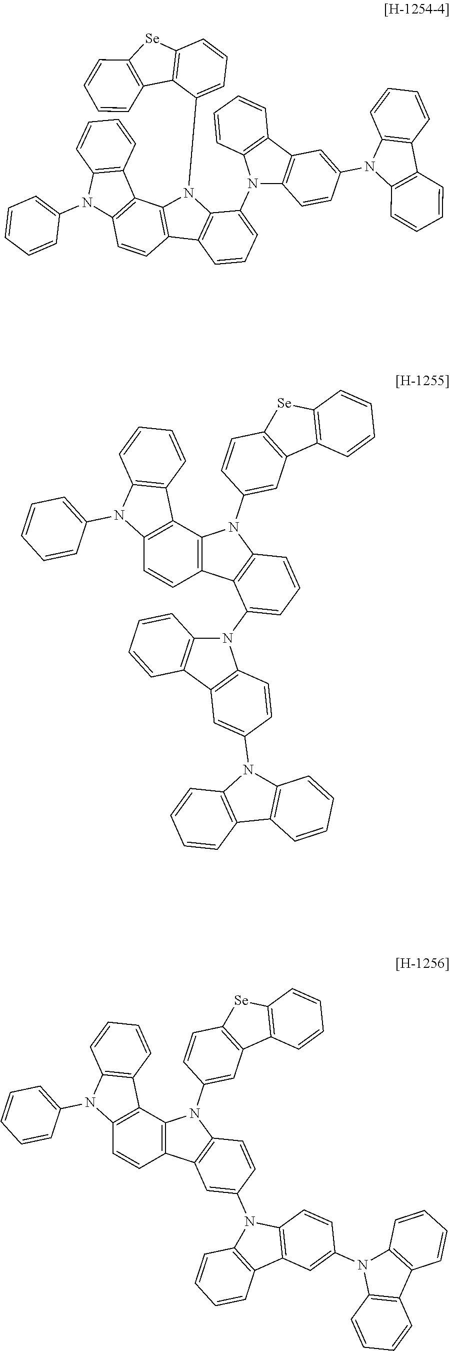 [SMILES: c1ccc(-n2c3ccccc3c3c2ccc2c4cccc(-n5c6ccccc6c6cc(-n7c8ccccc8c8ccccc87)ccc65)c4n(-c4cccc5[se]c6ccccc6c45)c23)cc1 ; c1ccc(-n2c3ccccc3c3c2ccc2c4c(-n5c6ccccc6c6cc(-n7c8ccccc8c8ccccc87)ccc65)cccc4n(-c4ccc5[se]c6ccccc6c5c4)c23)cc1 ; c1ccc(-n2c3ccccc3c3c2ccc2c4cc(-n5c6ccccc6c6cc(-n7c8ccccc8c8ccccc87)ccc65)ccc4n(-c4ccc5[se]c6ccccc6c5c4)c23)cc1]

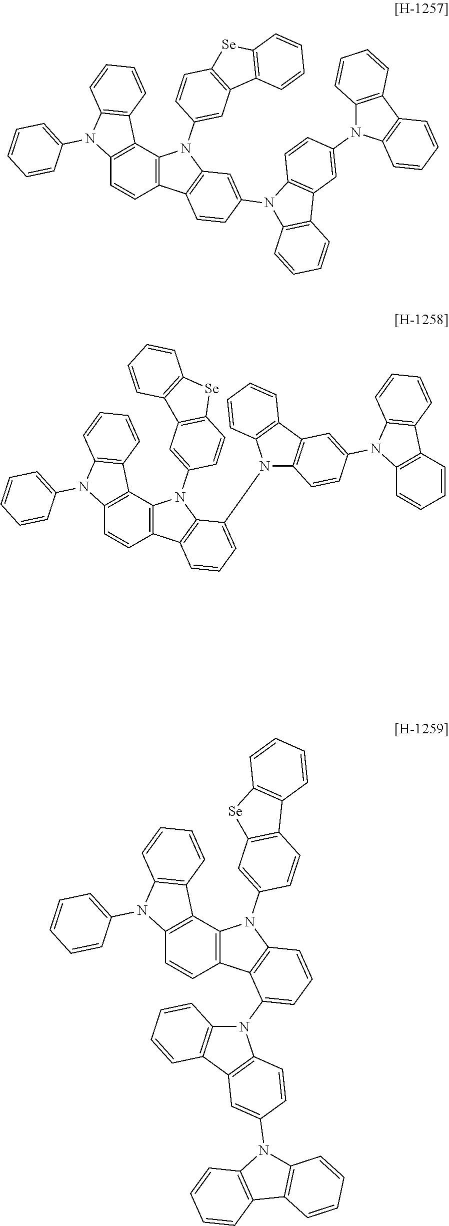 [SMILES: c1ccc(-n2c3ccccc3c3c2ccc2c4ccc(-n5c6ccccc6c6cc(-n7c8ccccc8c8ccccc87)ccc65)cc4n(-c4ccc5[se]c6ccccc6c5c4)c23)cc1 ; c1ccc(-n2c3ccccc3c3c2ccc2c4cccc(-n5c6ccccc6c6cc(-n7c8ccccc8c8ccccc87)ccc65)c4n(-c4ccc5[se]c6ccccc6c5c4)c23)cc1 ; c1ccc(-n2c3ccccc3c3c2ccc2c4c(-n5c6ccccc6c6cc(-n7c8ccccc8c8ccccc87)ccc65)cccc4n(-c4ccc5c(c4)[se]c4ccccc45)c23)cc1]

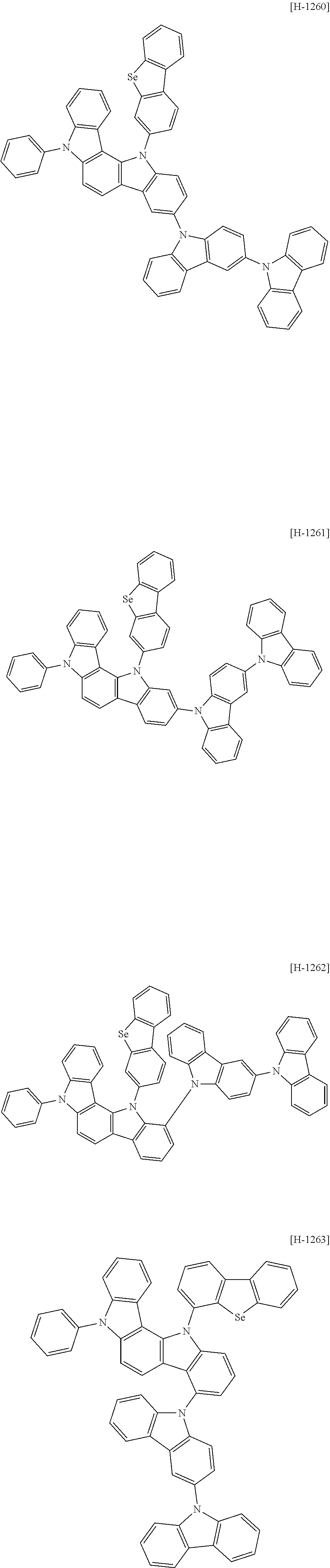 [SMILES: c1ccc(-n2c3ccccc3c3c2ccc2c4cc(-n5c6ccccc6c6cc(-n7c8ccccc8c8ccccc87)ccc65)ccc4n(-c4ccc5c(c4)[se]c4ccccc45)c23)cc1 ; c1ccc(-n2c3ccccc3c3c2ccc2c4ccc(-n5c6ccccc6c6cc(-n7c8ccccc8c8ccccc87)ccc65)cc4n(-c4ccc5c(c4)[se]c4ccccc45)c23)cc1 ; c1ccc(-n2c3ccccc3c3c2ccc2c4cccc(-n5c6ccccc6c6cc(-n7c8ccccc8c8ccccc87)ccc65)c4n(-c4ccc5c(c4)[se]c4ccccc45)c23)cc1 ; c1ccc(-n2c3ccccc3c3c2ccc2c4c(-n5c6ccccc6c6cc(-n7c8ccccc8c8ccccc87)ccc65)cccc4n(-c4cccc5c4[se]c4ccccc45)c23)cc1]

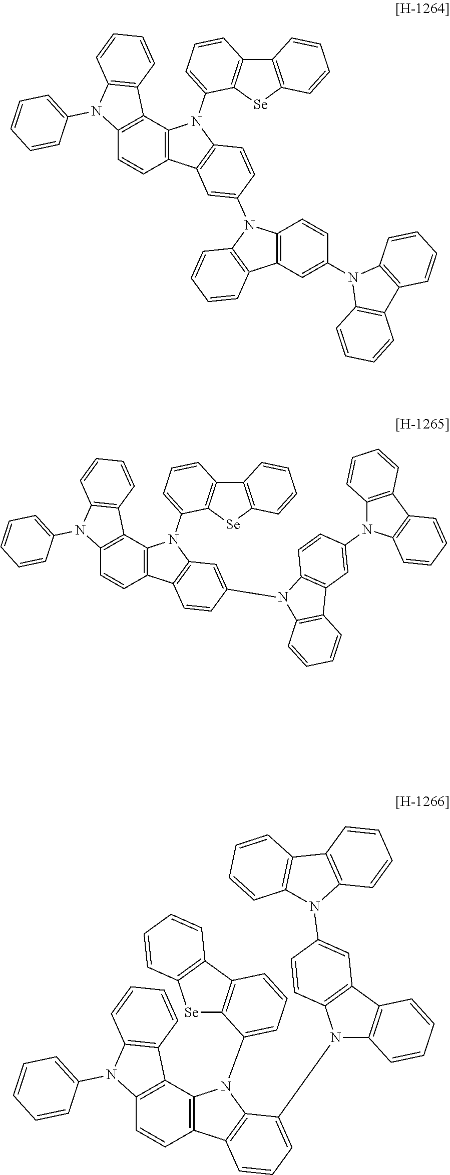 [SMILES: c1ccc(-n2c3ccccc3c3c2ccc2c4cc(-n5c6ccccc6c6cc(-n7c8ccccc8c8ccccc87)ccc65)ccc4n(-c4cccc5c4[se]c4ccccc45)c23)cc1 ; c1ccc(-n2c3ccccc3c3c2ccc2c4ccc(-n5c6ccccc6c6cc(-n7c8ccccc8c8ccccc87)ccc65)cc4n(-c4cccc5c4[se]c4ccccc45)c23)cc1 ; c1ccc(-n2c3ccccc3c3c2ccc2c4cccc(-n5c6ccccc6c6cc(-n7c8ccccc8c8ccccc87)ccc65)c4n(-c4cccc5c4[se]c4ccccc45)c23)cc1]

The aforementioned compound may be used as a composition together with other compounds to exhibit a synergistic effect in improving the performance of organic optoelectronic devices such as organic light emitting diodes.

A composition for an organic optoelectronic device according to some example embodiments may include the compound described above (the first compound) and a second compound different from the first compound. The second compound may include a substituted or unsubstituted nitrogen-containing six-membered ring moiety and may be represented, e.g., by Chemical Formula 2.

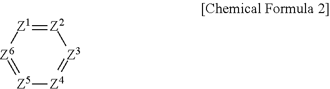

In Chemical Formula 2, Z¹ to Z⁶ may be each independently N or C-L^{a}-R^{e}, at least two of Z¹ to Z⁶ may be N, L^{a} may be each independently a single bond, a substituted or unsubstituted C6 to C20 arylene group, a substituted or unsubstituted divalent C2 to C20 heterocyclic group, or a combination thereof, R^{e} may be each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, and R^{e} may be each independently present, or two adjacent ones among Res may be linked to form a substituted or unsubstituted aliphatic, aromatic or heteroaromatic monocyclic or polycyclic ring.

The first compound and the second compound may be bipolar compounds having both electronic characteristics and hole characteristics, respectively. The first compound may be a bipolar compound with relatively strong hole characteristics, and the second compound may be a bipolar compound with relatively strong electronic characteristics. Additionally, the first compound and the second compound may exhibit good interfacial characteristics due to their structures.

The composition for an organic optoelectronic device includes the first compound and the second compound together to finely control the mobility of holes and electrons to balance holes and electrons in the active layer (e.g., light emitting layer) of the organic optoelectronic device.

In addition, the composition for an organic optoelectronic device may be used as a host for a light emitting layer, and has good electrical matching with a blue light-emitting dopant that emits light in a blue emission spectrum described later, thereby increasing efficiency of the organic optoelectronic device and suppressing deterioration of the organic optoelectronic device. As an example, at least one of the first compound and the second compound has a high triplet energy level of greater than or equal to about 2.8 eV, so that exciton transfer to the blue light-emitting dopant may be facilitated, thereby implementing an organic optoelectronic device having high efficiency and long life-span.

For example, in Chemical Formula 2, two of Z¹ to Z⁶ may be nitrogen (N) and the rest may be C-L^{a}-R^{e}.

For example, in Chemical Formula 2, Z¹ and Z³ may be nitrogen, Z² may be N or C-L^{a}-R^{e}, Z⁴ may be N or C-L^{a}-R^{e}, Z⁵ may be N or C-L^{a}-R^{e}, and Z⁶ may be N or C-L^{a}-R^{e}.

For example, in Chemical Formula 2, three of Z¹ to Z⁶ may be nitrogen (N) and the rest may be C-L^{a}-R^{e}.

For example, in Chemical Formula 2, Z¹, Z³, and Z⁵ may be nitrogen, Z² may be Nor C-L^{a}-R^{e}, Z⁴ may be N or C-L^{a}-R^{e} and Z⁶ may be N or C-L^{a}-R^{e}.

For example, the second compound represented by Chemical Formula 2 may be represented by any one of Chemical Formulas 2A to 2C, depending on the substituent of R^{e}.

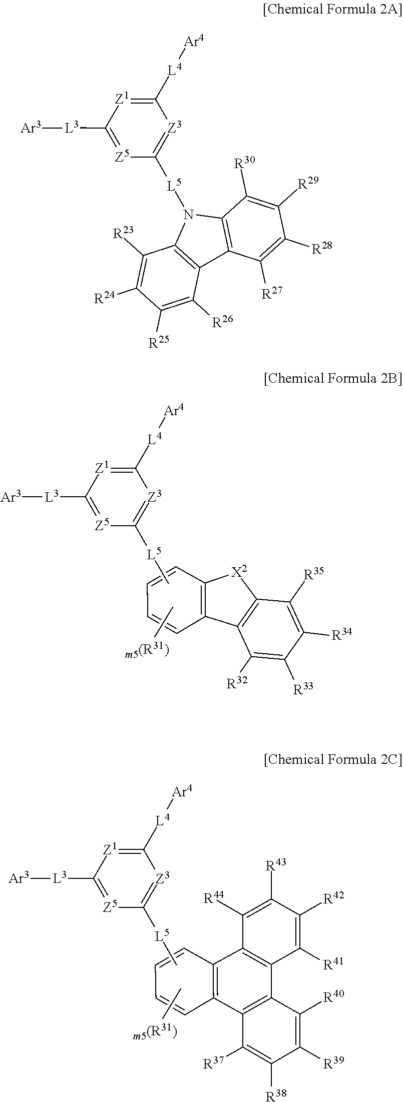

In Chemical Formula 2A to Chemical Formula 2C, Z¹, Z³, and Z⁵ may be each independently N or C-L^{a}-R^{e}, at least two of Z¹, Z³, and Z⁵ may be N, X² may be O, S, or NR^{f}, L^{a}, and L³ to L⁵ may be each independently a single bond, a substituted or unsubstituted C6 to C20 arylene group, a substituted or unsubstituted C2 to C20 heterocyclic group, or a combination thereof, R^{e}, R^{f} and R²³ to R⁴⁴ may be each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, R²³ to R³⁰ may be each independently present or two adjacent ones are linked to form a substituted or unsubstituted aromatic monocyclic or polycyclic ring, R³¹ to R³⁵ may be each independently present or two adjacent ones are linked to form a substituted or unsubstituted aromatic monocyclic or polycyclic ring, Ar³ and Ar⁴ may be each independently a substituted or unsubstituted C6 to C30 aryl group, a substituted or unsubstituted C2 to C30 heterocyclic group, or a combination thereof, R^{a}, Ar³, and Ar⁴ may be each independently present or two adjacent ones among R^{a}, Ar³, and Ar⁴ may be linked to form a substituted or unsubstituted aromatic or heteroaromatic monocyclic or polycyclic ring, and m5 and m6 are each independently one of integers of 1 to 3.

In Chemical Formula 2B, when m5 is 2 or more, each R³¹ may be the same or different. In Chemical Formula 2C, when m6 is 2 or more, each R³⁶ may be the same or different.

For example, the second compound represented by Chemical Formula 2A may be represented by any one of Chemical Formula 2A-I to Chemical Formula 2A-XVIII, below.

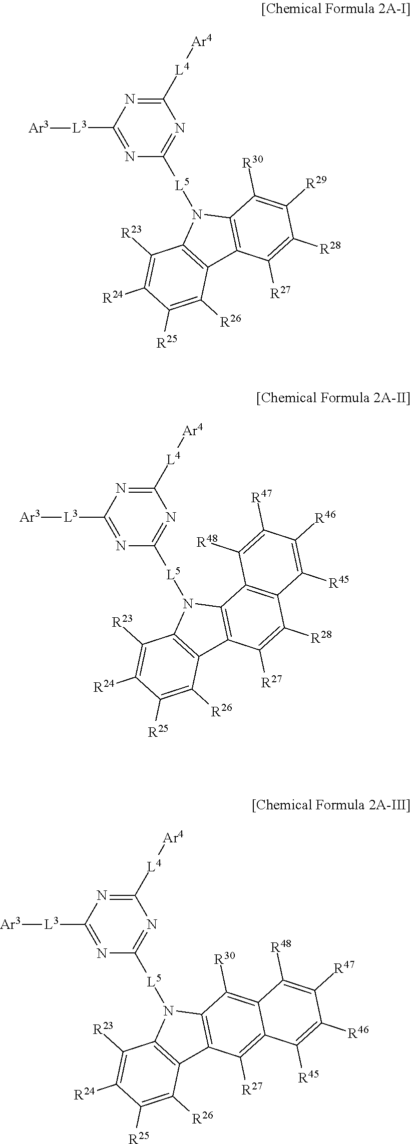

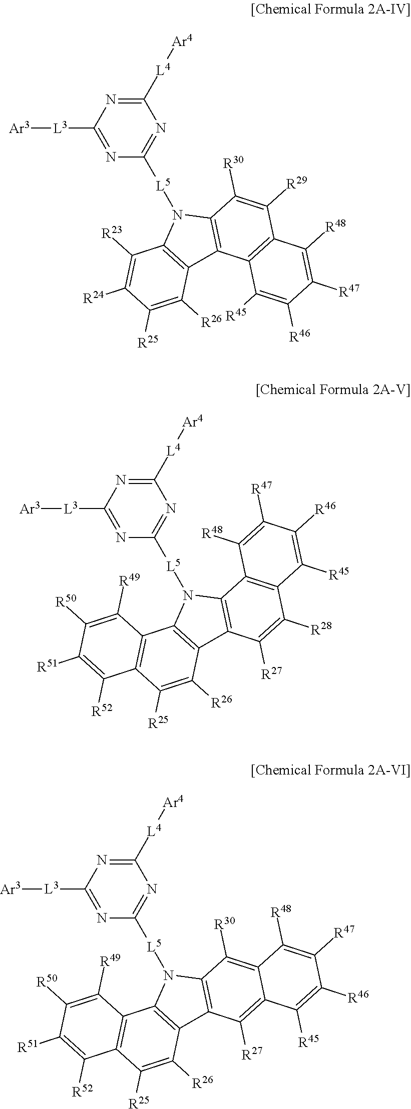

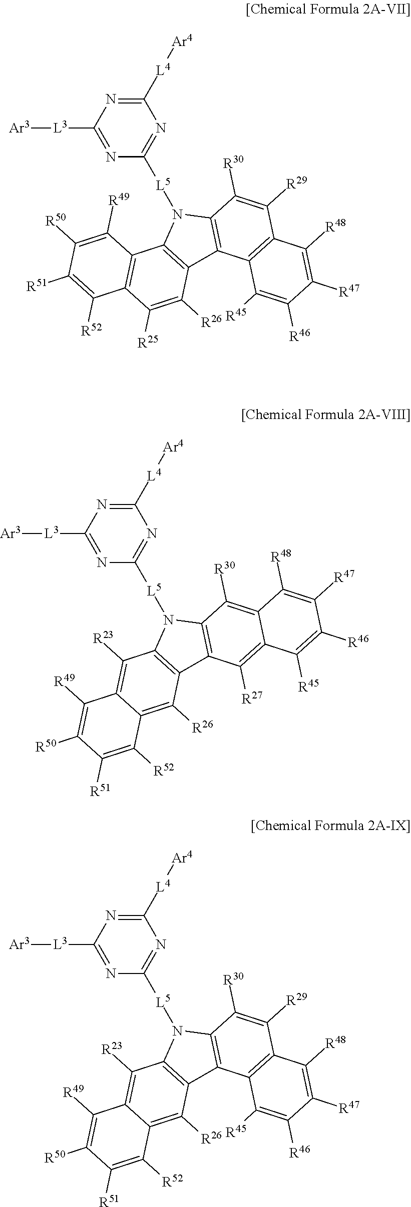

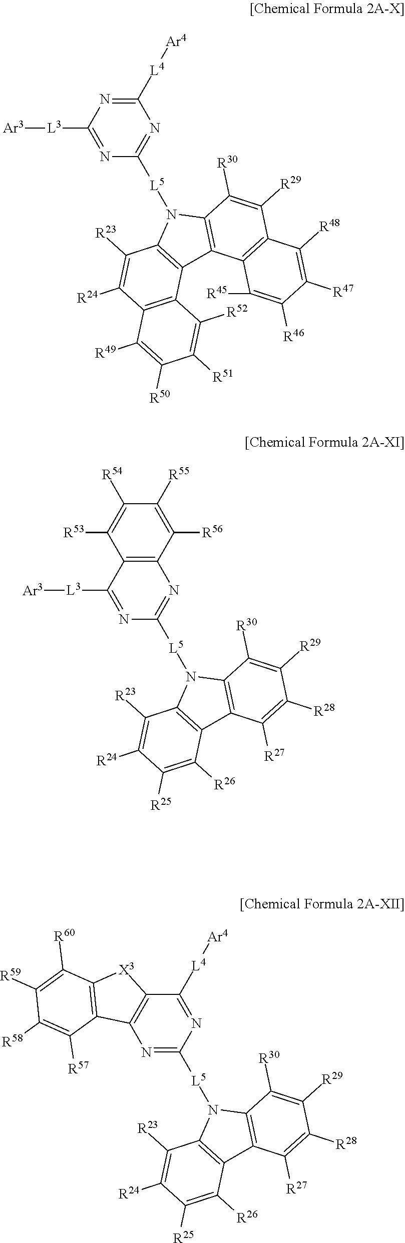

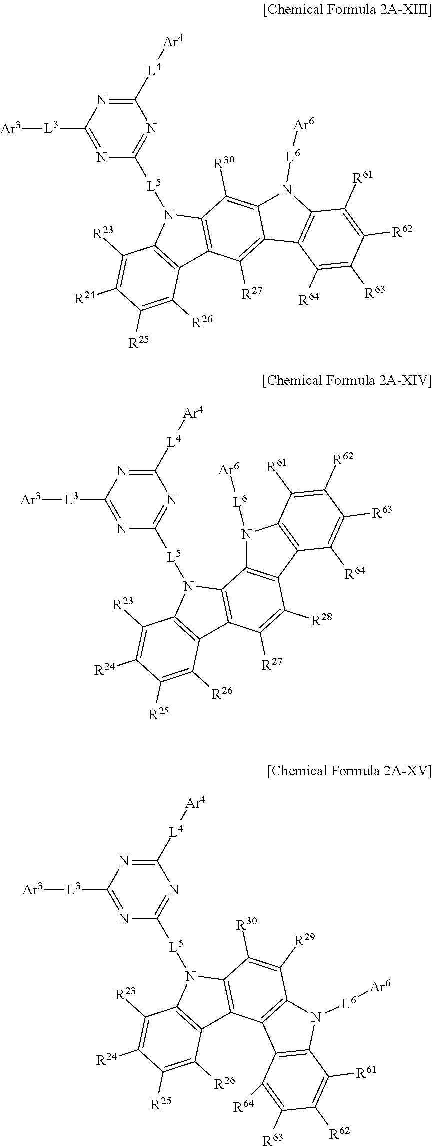

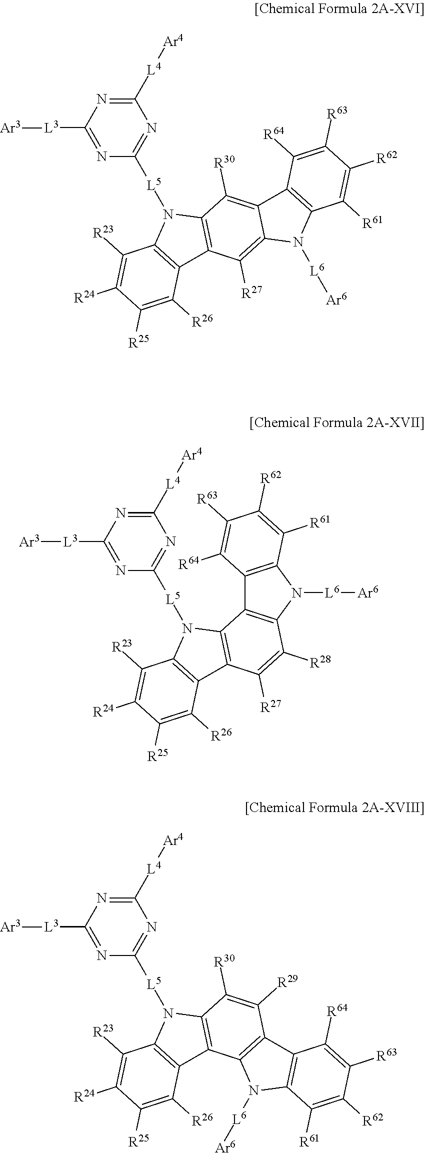

In Chemical Formula 2A-I to Chemical Formula 2A-XVIII, L² to L⁴, Ar³ and Art, and R²³ to R³⁰ may be the same as described above, X³ may be O or S, Ar⁶ may be a substituted or unsubstituted C6 to C30 aryl group or a substituted or unsubstituted C2 to C30 heterocyclic group, L⁶ may be a single bond, a substituted or unsubstituted C6 to C20 arylene group, or a substituted or unsubstituted C2 to C20 heterocyclic group, and R⁴⁵ to R⁶⁴ may be each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted C1 to C10 alkylsilyl group, a substituted or unsubstituted amine group, halogen, a cyano group, or a combination thereof.

For example, L³ to L⁶ may each independently be a single bond, a substituted or unsubstituted phenylene group, a substituted or unsubstituted biphenylene group, a substituted or unsubstituted naphthylene group, a substituted or unsubstituted dibenzofuranylene group, or a substituted or unsubstituted dibenzothiophenylene group.

For example, Ar³, Ar⁴, and Ar⁶ may each independently be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted triphenylene group, a substituted or unsubstituted carbazolyl group, a substituted or unsubstituted dibenzofuranyl group, or a substituted or unsubstituted dibenzothiophenyl group.

For example, R²³ to R³⁰ and R⁴⁵ to R⁶⁴ may each independently be hydrogen, deuterium, a cyano group, a substituted or unsubstituted C1 to C10 alkyl group, a substituted or unsubstituted C1 to C10 alkylsilyl group, a substituted or unsubstituted phenyl group, a substituted or unsubstituted dibenzofuranyl group, or a substituted or unsubstituted dibenzothiophenyl group.

For example, the second compound represented by Chemical Formula 2B may be represented by any one of Chemical Formula 2B-I to Chemical Formula 2B-IV, below.

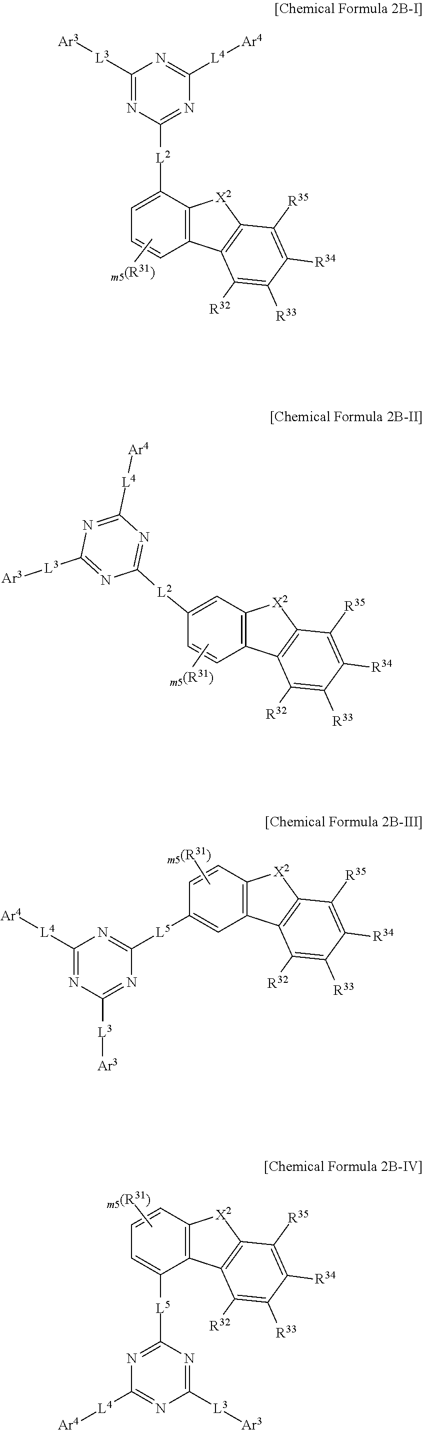

In Chemical Formula 2B-I to Chemical Formula 2B-IV, X², L³ to L⁵, Ar³ and Ar⁴, R³¹ to R³⁵, and m5 are the same as described above.

For example, a compound represented by Chemical Formula 2C may be represented by Chemical Formula 2C-I or Chemical Formula 2C-II, below.

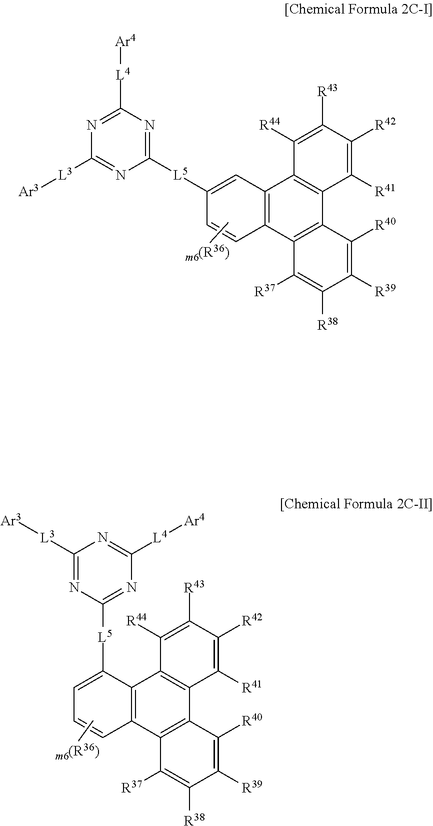

In Chemical Formula 2C-I and Chemical Formula 2C-II, L³ to L⁵, Ar³ and Ar⁴, R³⁶ to R⁴⁴, and m6 are the same as described above.

In some example embodiments, Chemical Formula 2 may be represented by Chemical Formula 2A-XIV or Chemical Formula 2C-I.

For example, in Chemical Formula 2A-XIV, L³ to L⁶ may each independently be a single bond or a substituted or unsubstituted C6 to C12 aryl group, Ar³, Ar⁴, and Ar⁶ may each independently be a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and R²³ to R²⁸ and R⁶¹ to R⁶⁴ may each independently be hydrogen, deuterium, a cyano group, a substituted or unsubstituted C1 to C10 alkyl group, a substituted or unsubstituted C1 to C10 alkylsilyl group, or a substituted or unsubstituted C6 to C12 aryl group.

For example, in Chemical Formula 2C-I, L³ to L⁵ may each independently be a single bond or a substituted or unsubstituted C6 to C12 aryl group, Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and R³⁶ to R⁴⁴ may each independently be hydrogen, deuterium, a cyano group, a substituted or unsubstituted C1 to C10 alkyl group, a substituted or unsubstituted C1 to C10 alkylsilyl group, or a substituted or unsubstituted C6 to C12 aryl group.

The second compound may be, e.g., one selected from the compounds listed in Group 2, below.

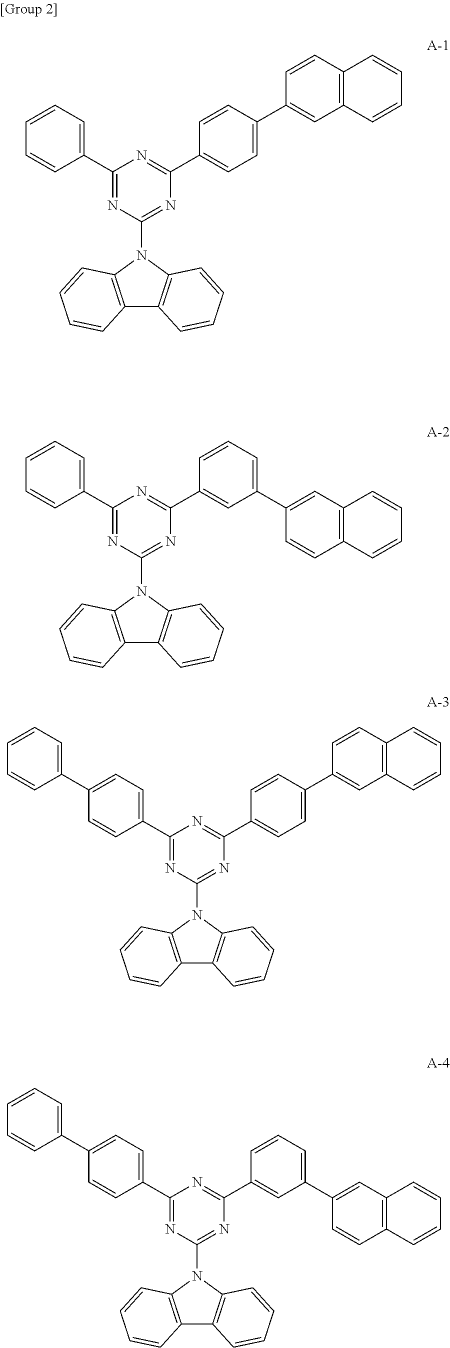 [SMILES: c1ccc(-c2nc(-c3ccc(-c4ccc5ccccc5c4)cc3)nc(-n3c4ccccc4c4ccccc43)n2)cc1 ; c1ccc(-c2nc(-c3cccc(-c4ccc5ccccc5c4)c3)nc(-n3c4ccccc4c4ccccc43)n2)cc1 ; c1ccc(-c2ccc(-c3nc(-c4ccc(-c5ccc6ccccc6c5)cc4)nc(-n4c5ccccc5c5ccccc54)n3)cc2)cc1 ; c1ccc(-c2ccc(-c3nc(-c4cccc(-c5ccc6ccccc6c5)c4)nc(-n4c5ccccc5c5ccccc54)n3)cc2)cc1]

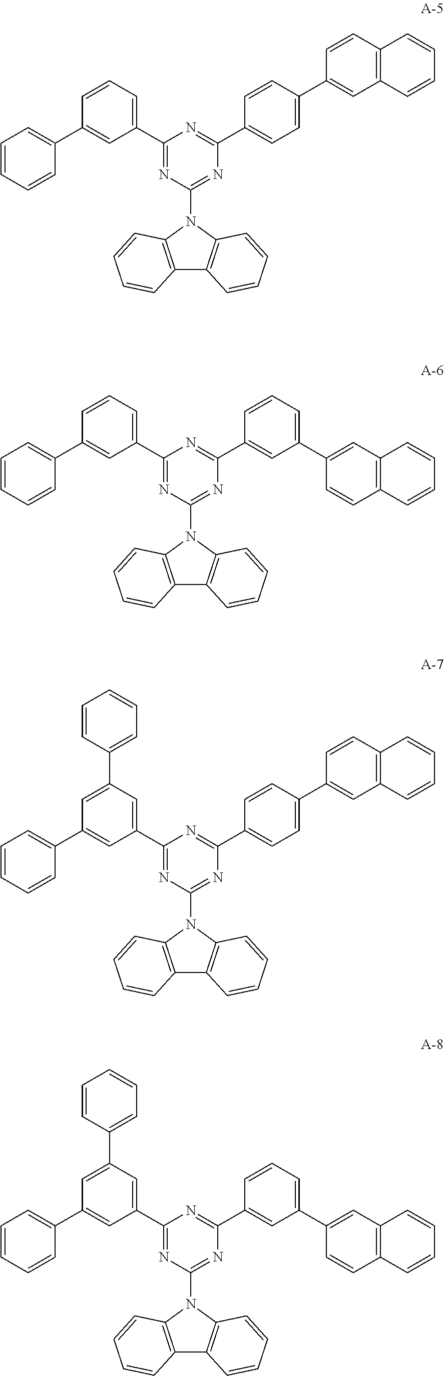 [SMILES: c1ccc(-c2cccc(-c3nc(-c4ccc(-c5ccc6ccccc6c5)cc4)nc(-n4c5ccccc5c5ccccc54)n3)c2)cc1 ; c1ccc(-c2cccc(-c3nc(-c4cccc(-c5ccc6ccccc6c5)c4)nc(-n4c5ccccc5c5ccccc54)n3)c2)cc1 ; c1ccc(-c2cc(-c3ccccc3)cc(-c3nc(-c4ccc(-c5ccc6ccccc6c5)cc4)nc(-n4c5ccccc5c5ccccc54)n3)c2)cc1 ; c1ccc(-c2cc(-c3ccccc3)cc(-c3nc(-c4cccc(-c5ccc6ccccc6c5)c4)nc(-n4c5ccccc5c5ccccc54)n3)c2)cc1]

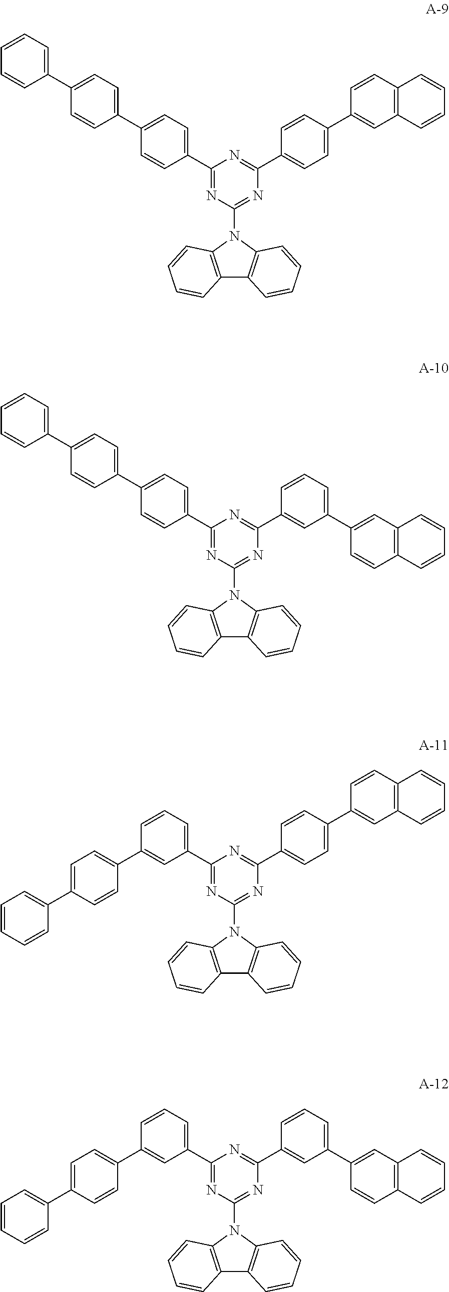 [SMILES: c1ccc(-c2ccc(-c3ccc(-c4nc(-c5ccc(-c6ccc7ccccc7c6)cc5)nc(-n5c6ccccc6c6ccccc65)n4)cc3)cc2)cc1 ; c1ccc(-c2ccc(-c3ccc(-c4nc(-c5cccc(-c6ccc7ccccc7c6)c5)nc(-n5c6ccccc6c6ccccc65)n4)cc3)cc2)cc1 ; c1ccc(-c2ccc(-c3cccc(-c4nc(-c5ccc(-c6ccc7ccccc7c6)cc5)nc(-n5c6ccccc6c6ccccc65)n4)c3)cc2)cc1 ; c1ccc(-c2ccc(-c3cccc(-c4nc(-c5cccc(-c6ccc7ccccc7c6)c5)nc(-n5c6ccccc6c6ccccc65)n4)c3)cc2)cc1]

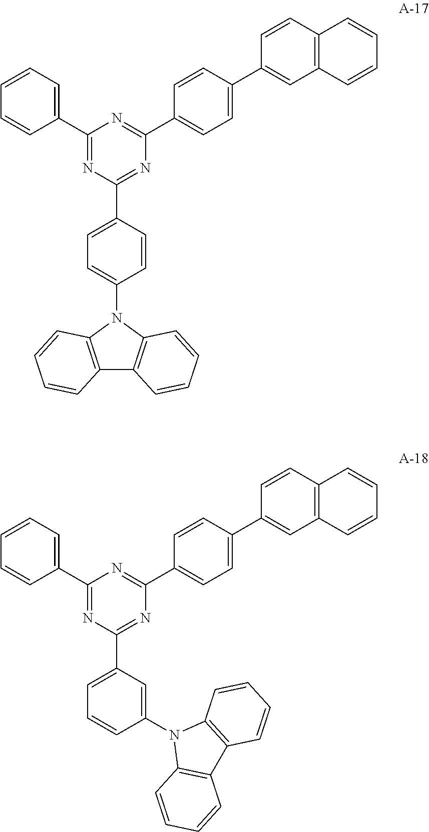 [SMILES: c1ccc(-c2nc(-c3ccc(-c4ccc5ccccc5c4)cc3)nc(-c3ccc(-n4c5ccccc5c5ccccc54)cc3)n2)cc1 ; c1ccc(-c2nc(-c3ccc(-c4ccc5ccccc5c4)cc3)nc(-c3cccc(-n4c5ccccc5c5ccccc54)c3)n2)cc1]

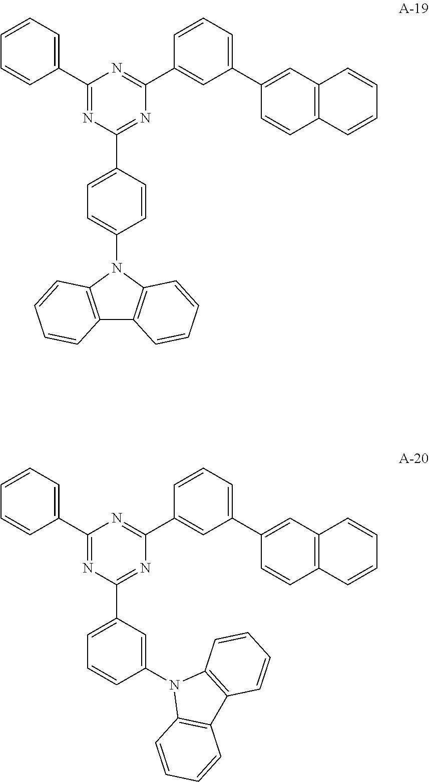 [SMILES: c1ccc(-c2nc(-c3ccc(-n4c5ccccc5c5ccccc54)cc3)nc(-c3cccc(-c4ccc5ccccc5c4)c3)n2)cc1 ; c1ccc(-c2nc(-c3cccc(-c4ccc5ccccc5c4)c3)nc(-c3cccc(-n4c5ccccc5c5ccccc54)c3)n2)cc1]

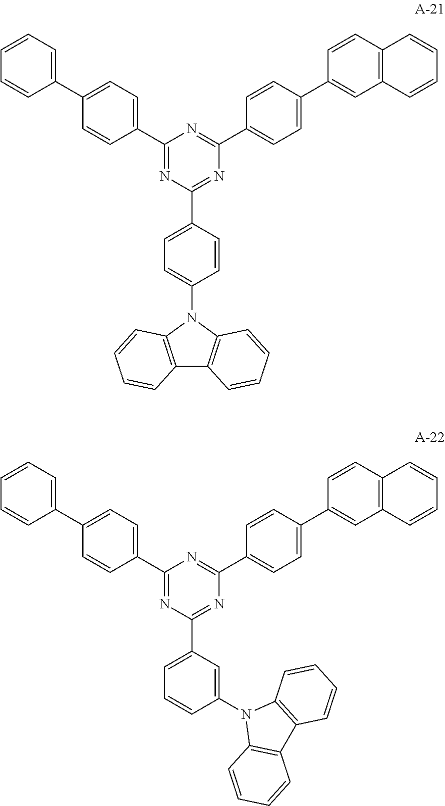 [SMILES: c1ccc(-c2ccc(-c3nc(-c4ccc(-c5ccc6ccccc6c5)cc4)nc(-c4ccc(-n5c6ccccc6c6ccccc65)cc4)n3)cc2)cc1 ; c1ccc(-c2ccc(-c3nc(-c4ccc(-c5ccc6ccccc6c5)cc4)nc(-c4cccc(-n5c6ccccc6c6ccccc65)c4)n3)cc2)cc1]

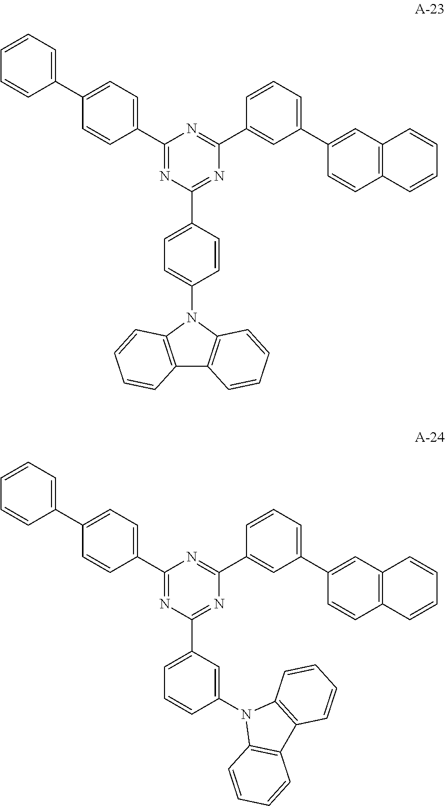 [SMILES: c1ccc(-c2ccc(-c3nc(-c4ccc(-n5c6ccccc6c6ccccc65)cc4)nc(-c4cccc(-c5ccc6ccccc6c5)c4)n3)cc2)cc1 ; c1ccc(-c2ccc(-c3nc(-c4cccc(-c5ccc6ccccc6c5)c4)nc(-c4cccc(-n5c6ccccc6c6ccccc65)c4)n3)cc2)cc1]

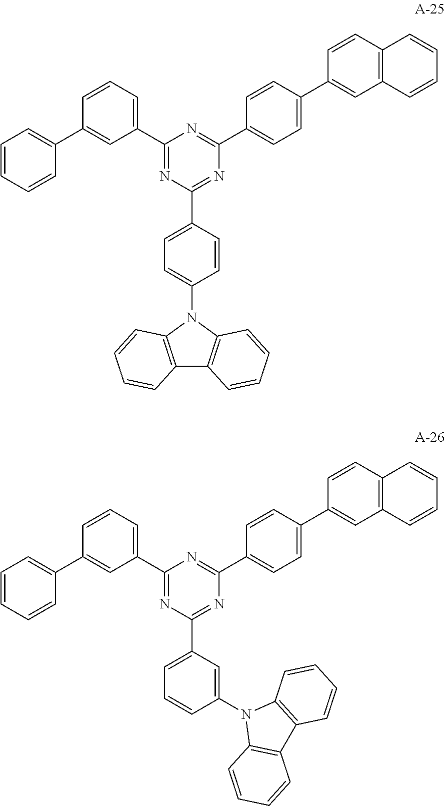 [SMILES: c1ccc(-c2cccc(-c3nc(-c4ccc(-c5ccc6ccccc6c5)cc4)nc(-c4ccc(-n5c6ccccc6c6ccccc65)cc4)n3)c2)cc1 ; c1ccc(-c2cccc(-c3nc(-c4ccc(-c5ccc6ccccc6c5)cc4)nc(-c4cccc(-n5c6ccccc6c6ccccc65)c4)n3)c2)cc1]

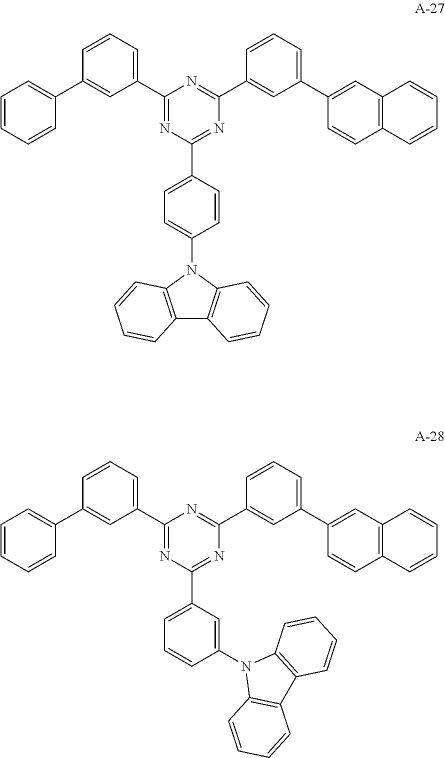 [SMILES: c1ccc(-c2cccc(-c3nc(-c4ccc(-n5c6ccccc6c6ccccc65)cc4)nc(-c4cccc(-c5ccc6ccccc6c5)c4)n3)c2)cc1 ; c1ccc(-c2cccc(-c3nc(-c4cccc(-c5ccc6ccccc6c5)c4)nc(-c4cccc(-n5c6ccccc6c6ccccc65)c4)n3)c2)cc1]

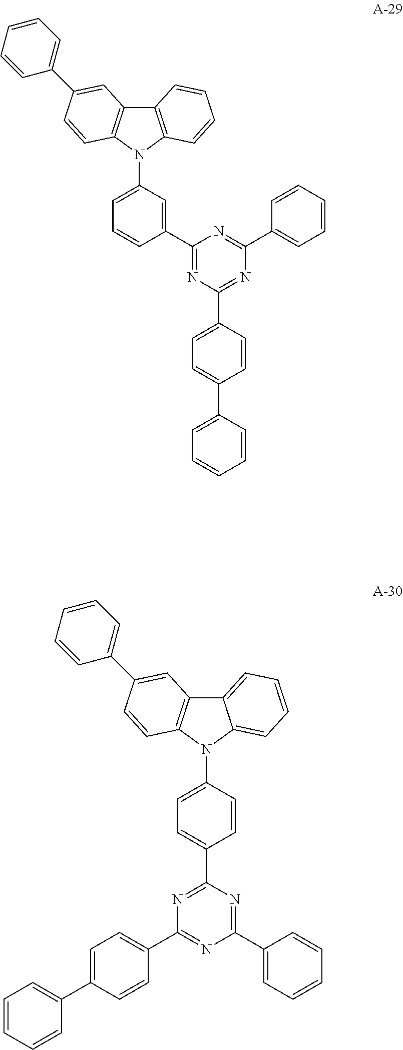 [SMILES: c1ccc(-c2ccc(-c3nc(-c4ccccc4)nc(-c4cccc(-n5c6ccccc6c6cc(-c7ccccc7)ccc65)c4)n3)cc2)cc1 ; c1ccc(-c2ccc(-c3nc(-c4ccccc4)nc(-c4ccc(-n5c6ccccc6c6cc(-c7ccccc7)ccc65)cc4)n3)cc2)cc1]

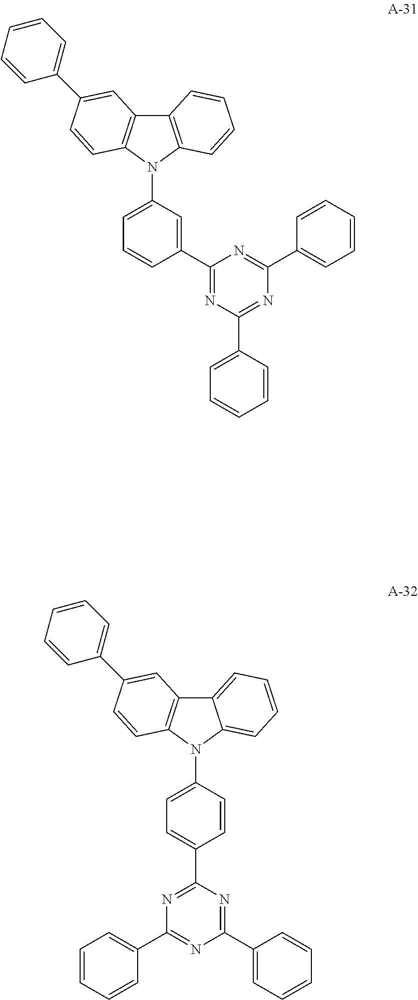 [SMILES: c1ccc(-c2ccc3c(c2)c2ccccc2n3-c2cccc(-c3nc(-c4ccccc4)nc(-c4ccccc4)n3)c2)cc1 ; c1ccc(-c2ccc3c(c2)c2ccccc2n3-c2ccc(-c3nc(-c4ccccc4)nc(-c4ccccc4)n3)cc2)cc1]

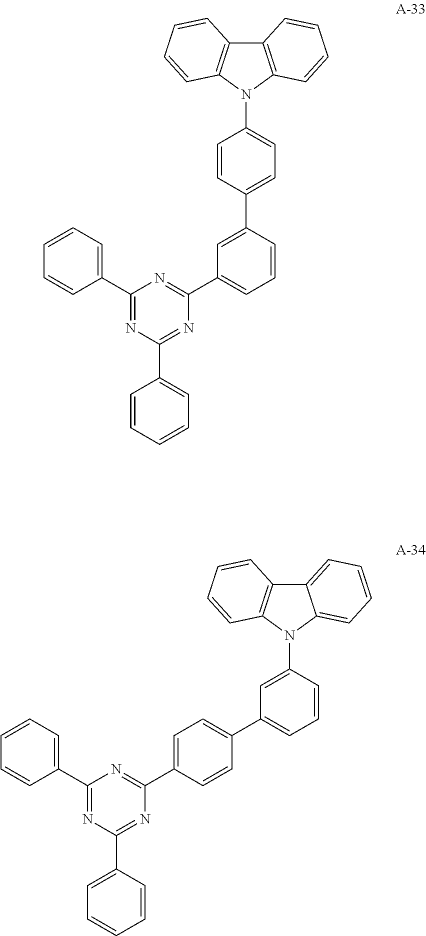 [SMILES: c1ccc(-c2nc(-c3ccccc3)nc(-c3cccc(-c4ccc(-n5c6ccccc6c6ccccc65)cc4)c3)n2)cc1 ; c1ccc(-c2nc(-c3ccccc3)nc(-c3ccc(-c4cccc(-n5c6ccccc6c6ccccc65)c4)cc3)n2)cc1]

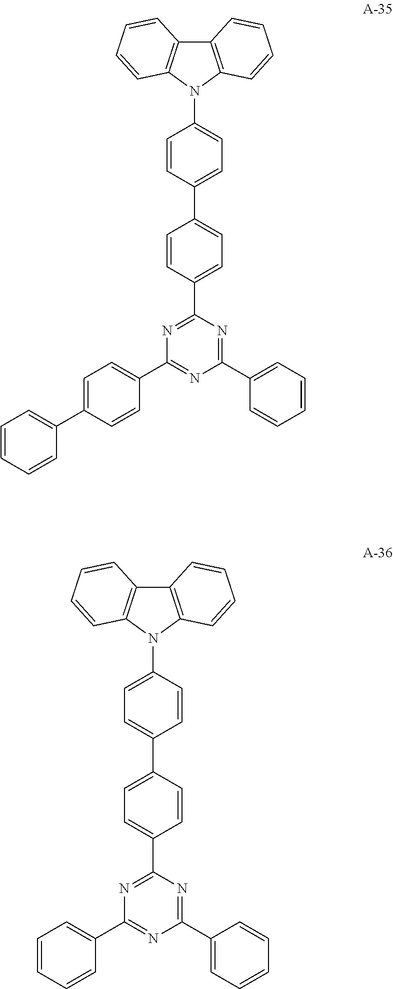 [SMILES: c1ccc(-c2ccc(-c3nc(-c4ccccc4)nc(-c4ccc(-c5ccc(-n6c7ccccc7c7ccccc76)cc5)cc4)n3)cc2)cc1 ; c1ccc(-c2nc(-c3ccccc3)nc(-c3ccc(-c4ccc(-n5c6ccccc6c6ccccc65)cc4)cc3)n2)cc1]

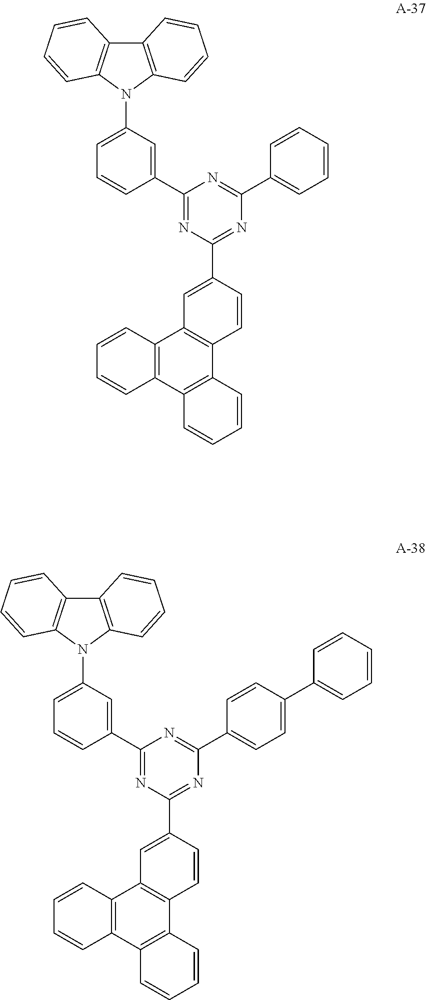 [SMILES: c1ccc(-c2nc(-c3cccc(-n4c5ccccc5c5ccccc54)c3)nc(-c3ccc4c5ccccc5c5ccccc5c4c3)n2)cc1 ; c1ccc(-c2ccc(-c3nc(-c4cccc(-n5c6ccccc6c6ccccc65)c4)nc(-c4ccc5c6ccccc6c6ccccc6c5c4)n3)cc2)cc1]

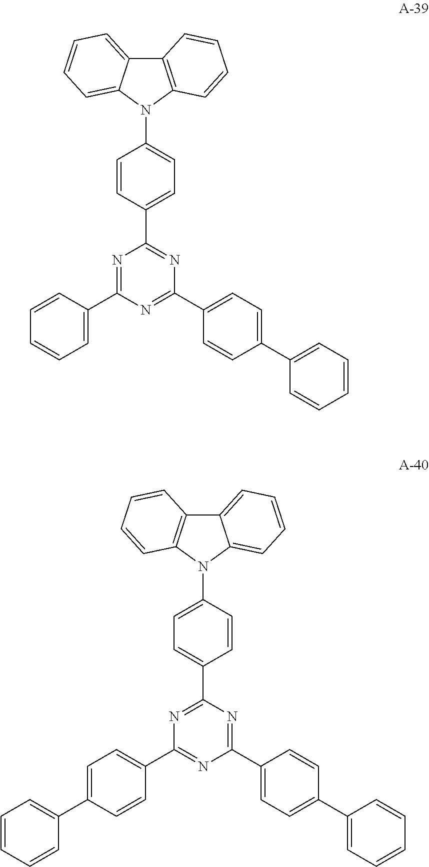 [SMILES: c1ccc(-c2ccc(-c3nc(-c4ccccc4)nc(-c4ccc(-n5c6ccccc6c6ccccc65)cc4)n3)cc2)cc1 ; c1ccc(-c2ccc(-c3nc(-c4ccc(-c5ccccc5)cc4)nc(-c4ccc(-n5c6ccccc6c6ccccc65)cc4)n3)cc2)cc1]

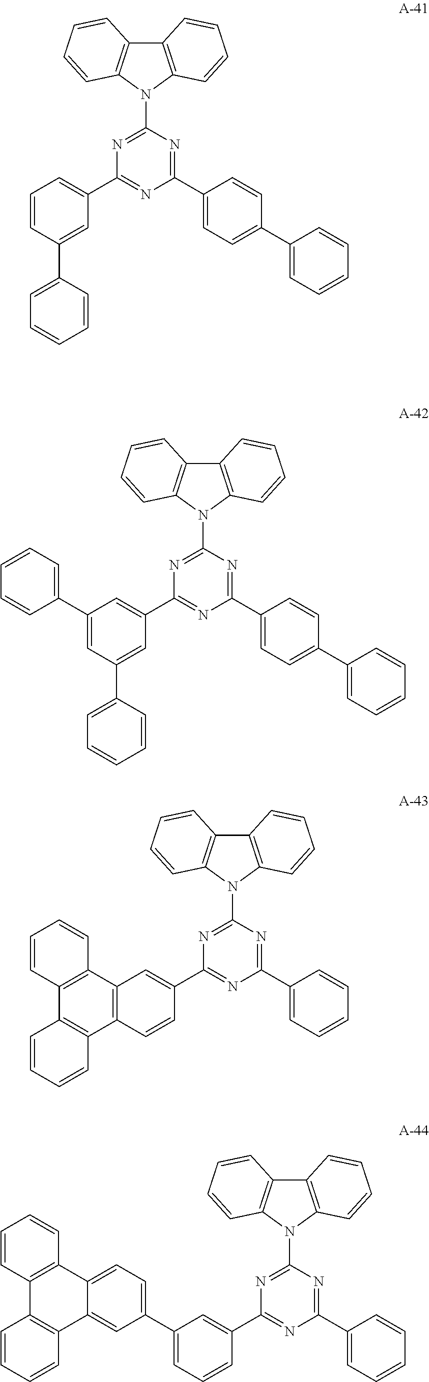 [SMILES: c1ccc(-c2ccc(-c3nc(-c4cccc(-c5ccccc5)c4)nc(-n4c5ccccc5c5ccccc54)n3)cc2)cc1 ; c1ccc(-c2ccc(-c3nc(-c4cc(-c5ccccc5)cc(-c5ccccc5)c4)nc(-n4c5ccccc5c5ccccc54)n3)cc2)cc1 ; c1ccc(-c2nc(-c3ccc4c5ccccc5c5ccccc5c4c3)nc(-n3c4ccccc4c4ccccc43)n2)cc1 ; c1ccc(-c2nc(-c3cccc(-c4ccc5c6ccccc6c6ccccc6c5c4)c3)nc(-n3c4ccccc4c4ccccc43)n2)cc1]

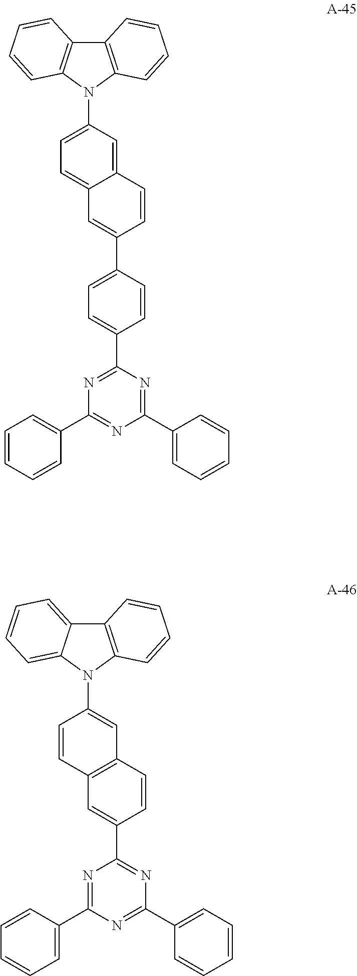 [SMILES: c1ccc(-c2nc(-c3ccccc3)nc(-c3ccc(-c4ccc5cc(-n6c7ccccc7c7ccccc76)ccc5c4)cc3)n2)cc1 ; c1ccc(-c2nc(-c3ccccc3)nc(-c3ccc4cc(-n5c6ccccc6c6ccccc65)ccc4c3)n2)cc1]

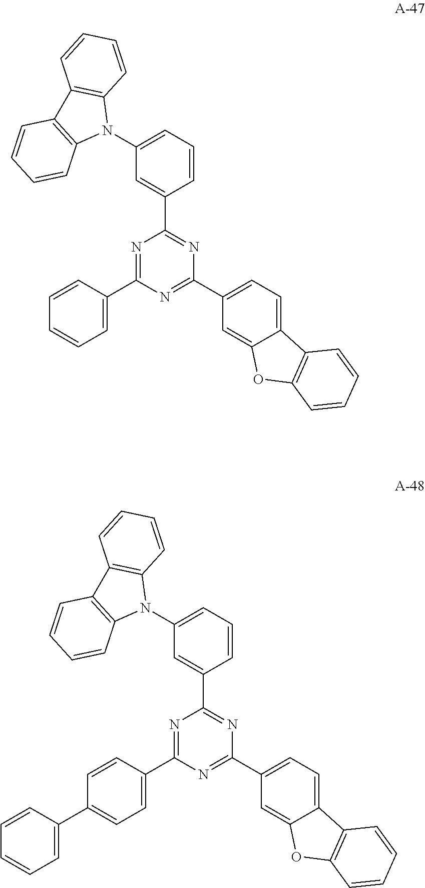 [SMILES: c1ccc(-c2nc(-c3cccc(-n4c5ccccc5c5ccccc54)c3)nc(-c3ccc4c(c3)oc3ccccc34)n2)cc1 ; c1ccc(-c2ccc(-c3nc(-c4cccc(-n5c6ccccc6c6ccccc65)c4)nc(-c4ccc5c(c4)oc4ccccc45)n3)cc2)cc1]

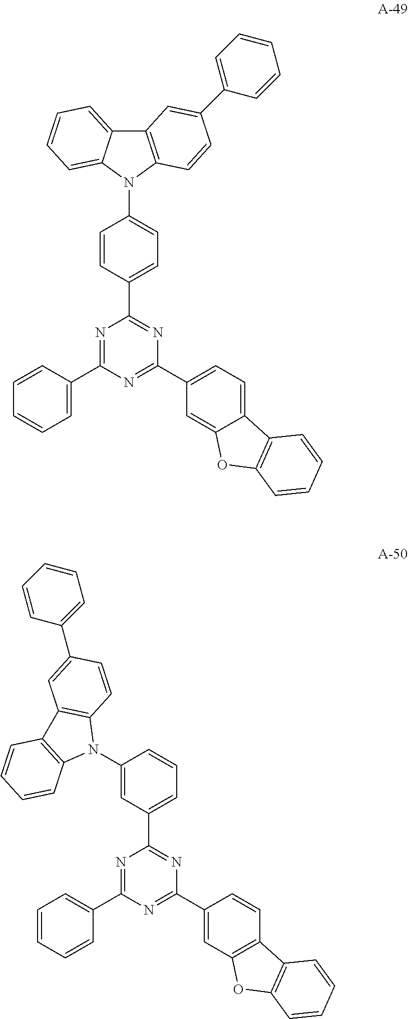 [SMILES: c1ccc(-c2ccc3c(c2)c2ccccc2n3-c2ccc(-c3nc(-c4ccccc4)nc(-c4ccc5c(c4)oc4ccccc45)n3)cc2)cc1 ; c1ccc(-c2ccc3c(c2)c2ccccc2n3-c2cccc(-c3nc(-c4ccccc4)nc(-c4ccc5c(c4)oc4ccccc45)n3)c2)cc1]

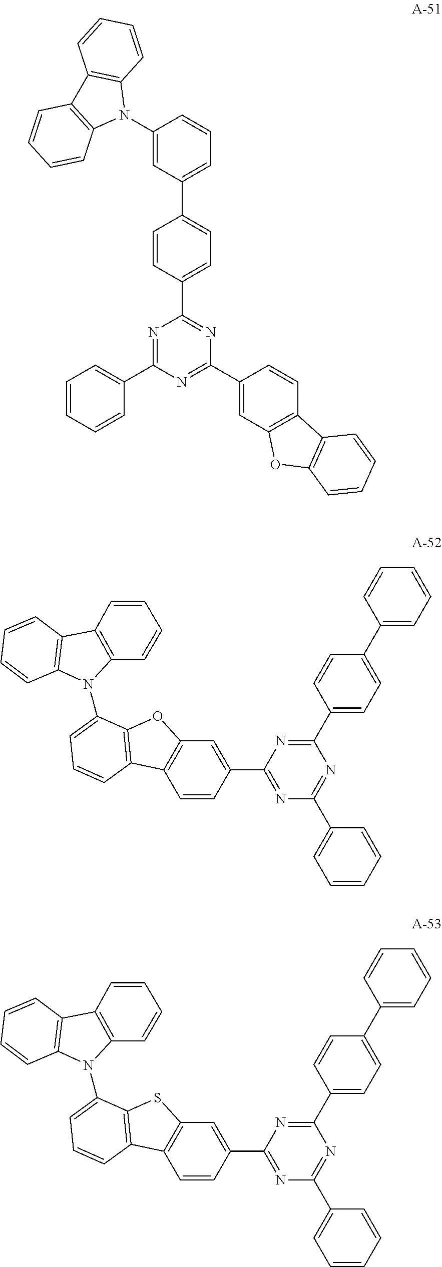 [SMILES: c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2ccc(-c3nc(-c4ccccc4)nc(-c4ccc5c(c4)oc4c(-n6c7ccccc7c7ccccc76)cccc45)n3)cc2)cc1 ; c1ccc(-c2ccc(-c3nc(-c4ccccc4)nc(-c4ccc5c(c4)sc4c(-n6c7ccccc7c7ccccc76)cccc45)n3)cc2)cc1]

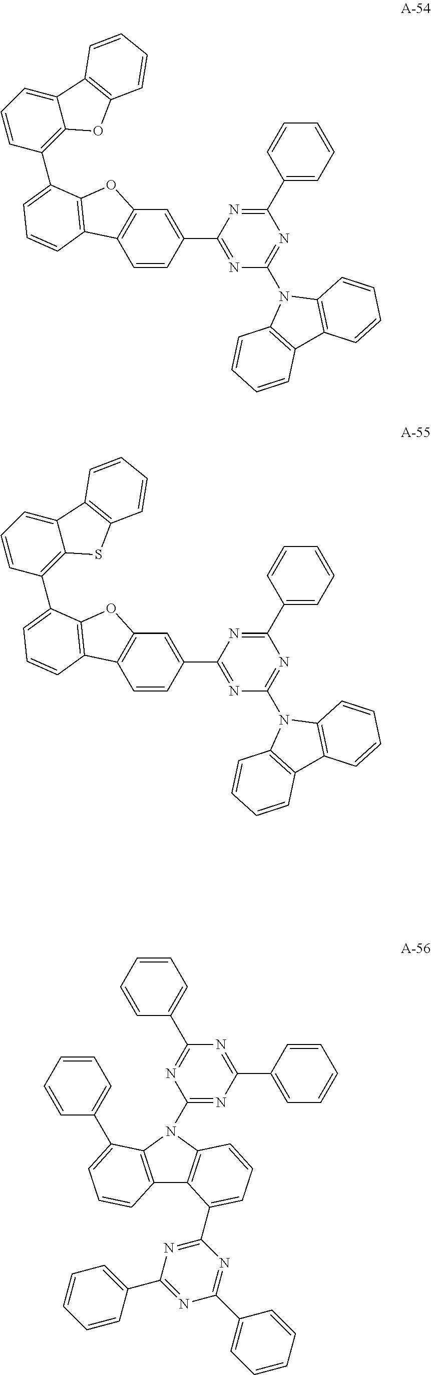 [SMILES: c1ccc(-c2nc(-c3ccc4c(c3)oc3c(-c5cccc6c5oc5ccccc56)cccc34)nc(-n3c4ccccc4c4ccccc43)n2)cc1 ; c1ccc(-c2nc(-c3ccc4c(c3)oc3c(-c5cccc6c5sc5ccccc56)cccc34)nc(-n3c4ccccc4c4ccccc43)n2)cc1 ; c1ccc(-c2nc(-c3ccccc3)nc(-c3cccc4c3c3cccc(-c5ccccc5)c3n4-c3nc(-c4ccccc4)nc(-c4ccccc4)n3)n2)cc1]

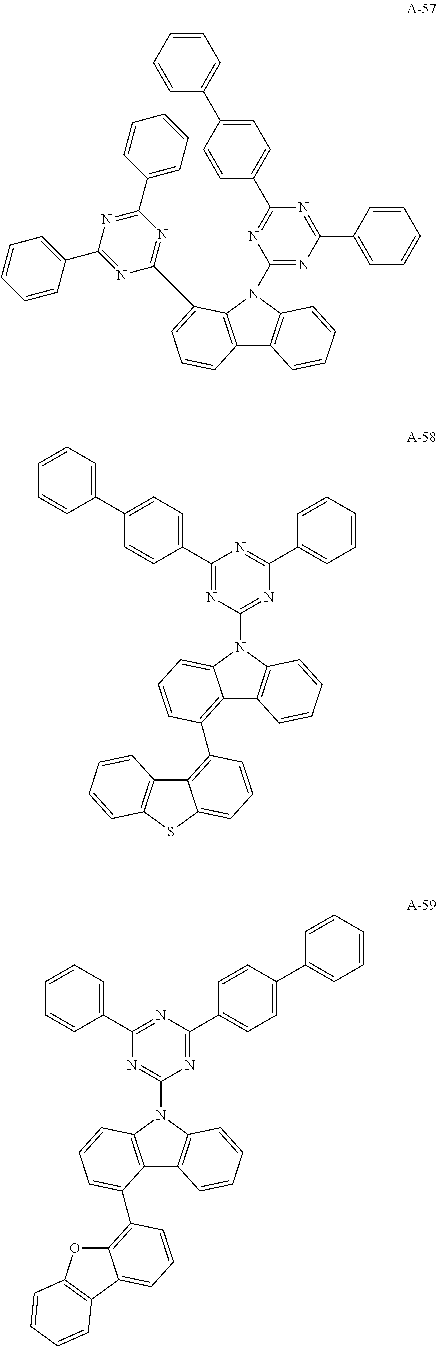 [SMILES: c1ccc(-c2ccc(-c3nc(-c4ccccc4)nc(-n4c5ccccc5c5cccc(-c6nc(-c7ccccc7)nc(-c7ccccc7)n6)c54)n3)cc2)cc1 ; c1ccc(-c2ccc(-c3nc(-c4ccccc4)nc(-n4c5ccccc5c5c(-c6cccc7sc8ccccc8c67)cccc54)n3)cc2)cc1 ; c1ccc(-c2ccc(-c3nc(-c4ccccc4)nc(-n4c5ccccc5c5c(-c6cccc7c6oc6ccccc67)cccc54)n3)cc2)cc1]

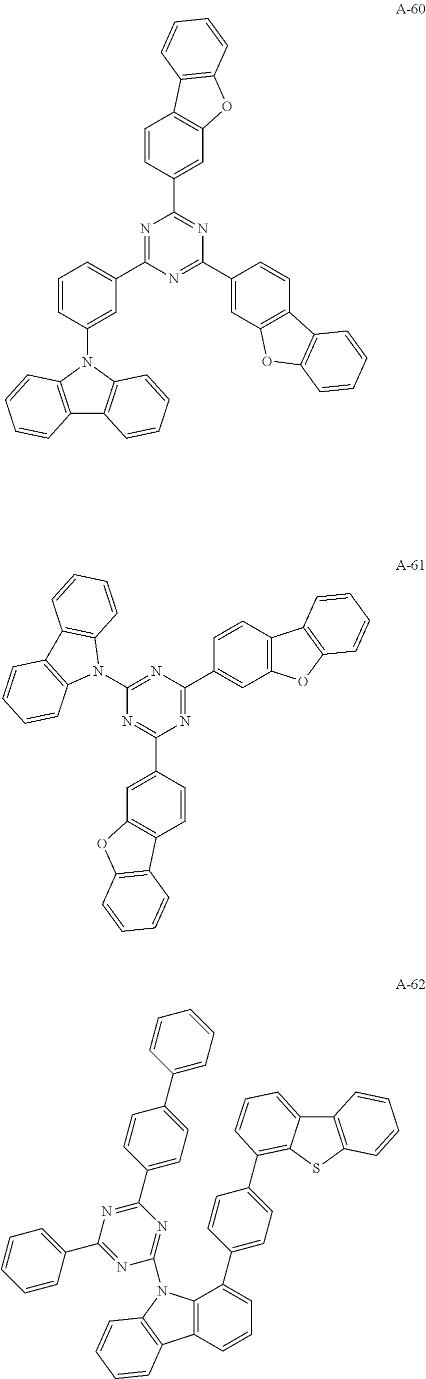 [SMILES: c1cc(-c2nc(-c3ccc4c(c3)oc3ccccc34)nc(-c3ccc4c(c3)oc3ccccc34)n2)cc(-n2c3ccccc3c3ccccc32)c1 ; c1ccc2c(c1)oc1cc(-c3nc(-c4ccc5c(c4)oc4ccccc45)nc(-n4c5ccccc5c5ccccc54)n3)ccc12 ; c1ccc(-c2ccc(-c3nc(-c4ccccc4)nc(-n4c5ccccc5c5cccc(-c6ccc(-c7cccc8c7sc7ccccc78)cc6)c54)n3)cc2)cc1]

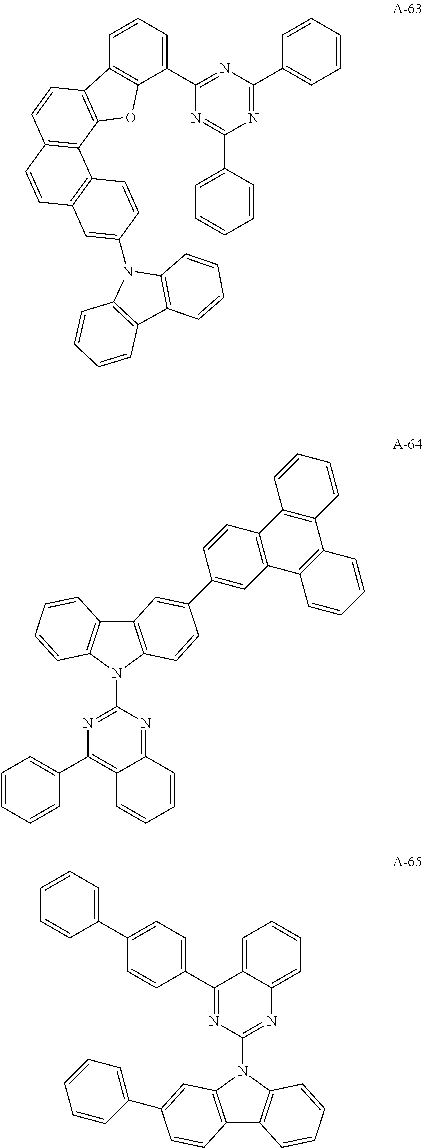 [SMILES: c1ccc(-c2nc(-c3ccccc3)nc(-c3cccc4c3oc3c4ccc4ccc5cc(-n6c7ccccc7c7ccccc76)ccc5c43)n2)cc1 ; c1ccc(-c2nc(-n3c4ccccc4c4cc(-c5ccc6c7ccccc7c7ccccc7c6c5)ccc43)nc3ccccc23)cc1 ; c1ccc(-c2ccc(-c3nc(-n4c5ccccc5c5ccc(-c6ccccc6)cc54)nc4ccccc34)cc2)cc1]

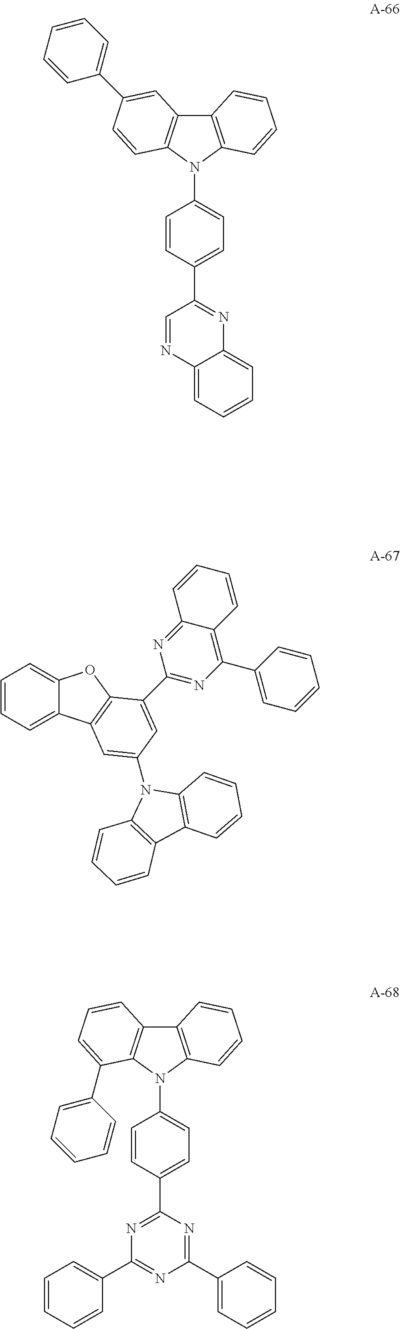 [SMILES: c1ccc(-c2ccc3c(c2)c2ccccc2n3-c2ccc(-c3cnc4ccccc4n3)cc2)cc1 ; c1ccc(-c2nc(-c3cc(-n4c5ccccc5c5ccccc54)cc4c3oc3ccccc34)nc3ccccc23)cc1 ; c1ccc(-c2nc(-c3ccccc3)nc(-c3ccc(-n4c5ccccc5c5cccc(-c6ccccc6)c54)cc3)n2)cc1]

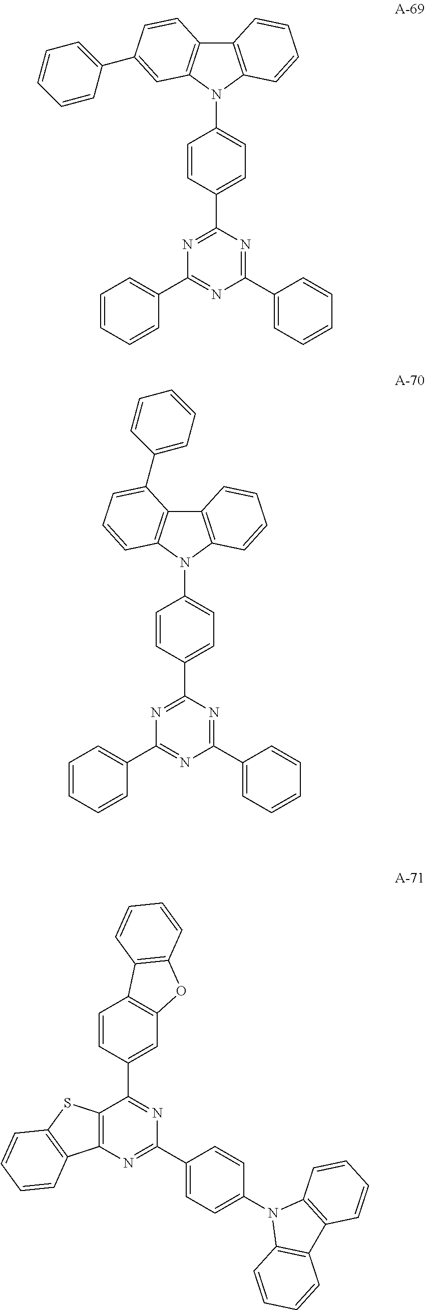 [SMILES: c1ccc(-c2ccc3c4ccccc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5c(-c6ccccc6)cccc54)cc3)n2)cc1 ; c1ccc2c(c1)oc1cc(-c3nc(-c4ccc(-n5c6ccccc6c6ccccc65)cc4)nc4c3sc3ccccc34)ccc12]

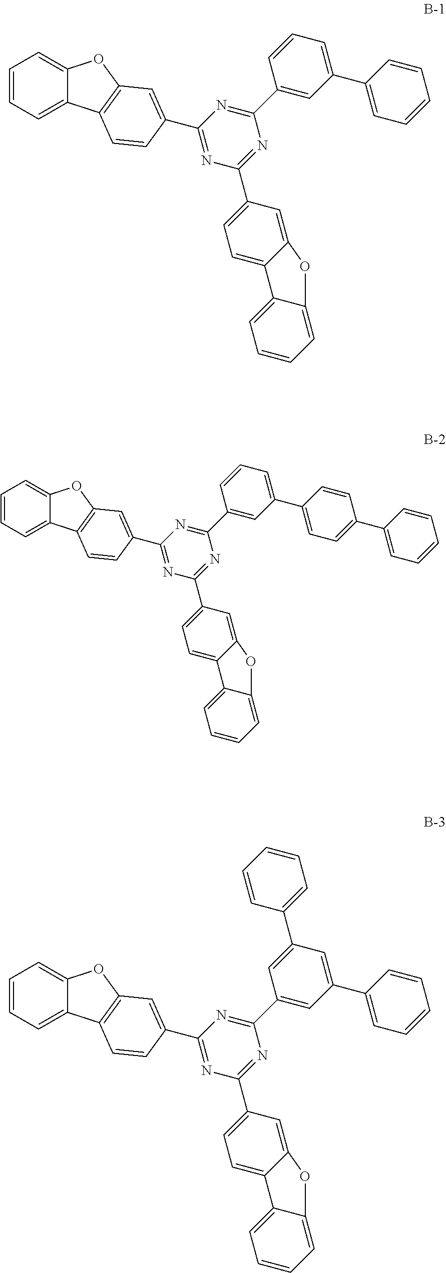 [SMILES: c1ccc(-c2cccc(-c3nc(-c4ccc5c(c4)oc4ccccc45)nc(-c4ccc5c(c4)oc4ccccc45)n3)c2)cc1 ; c1ccc(-c2ccc(-c3cccc(-c4nc(-c5ccc6c(c5)oc5ccccc56)nc(-c5ccc6c(c5)oc5ccccc56)n4)c3)cc2)cc1 ; c1ccc(-c2cc(-c3ccccc3)cc(-c3nc(-c4ccc5c(c4)oc4ccccc45)nc(-c4ccc5c(c4)oc4ccccc45)n3)c2)cc1]

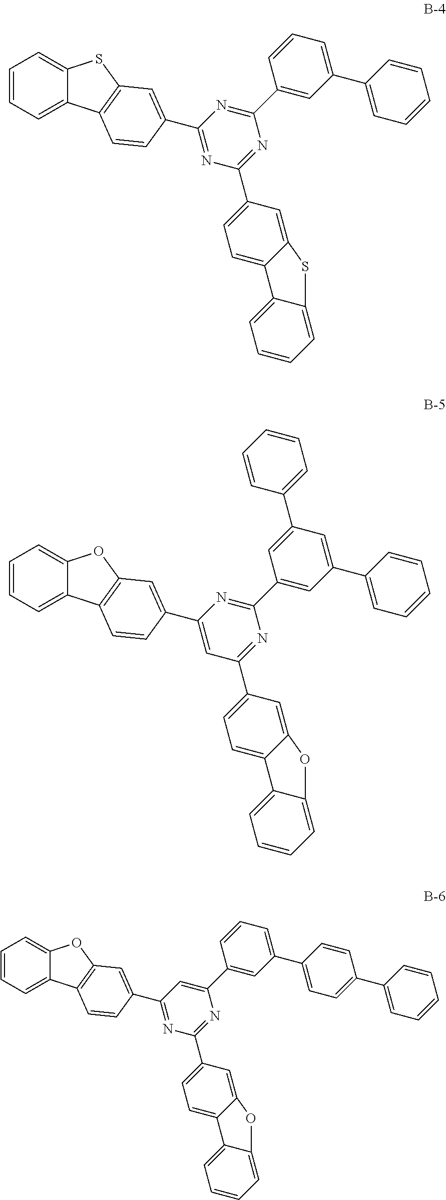 [SMILES: c1ccc(-c2cccc(-c3nc(-c4ccc5c(c4)sc4ccccc45)nc(-c4ccc5c(c4)sc4ccccc45)n3)c2)cc1 ; c1ccc(-c2cc(-c3ccccc3)cc(-c3nc(-c4ccc5c(c4)oc4ccccc45)cc(-c4ccc5c(c4)oc4ccccc45)n3)c2)cc1 ; c1ccc(-c2ccc(-c3cccc(-c4cc(-c5ccc6c(c5)oc5ccccc56)nc(-c5ccc6c(c5)oc5ccccc56)n4)c3)cc2)cc1]

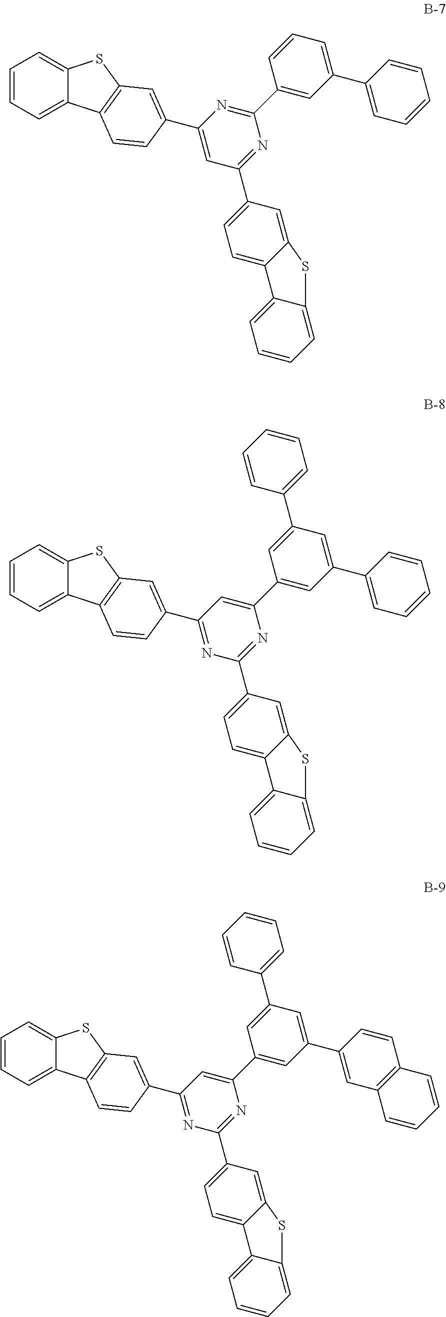 [SMILES: c1ccc(-c2cccc(-c3nc(-c4ccc5c(c4)sc4ccccc45)cc(-c4ccc5c(c4)sc4ccccc45)n3)c2)cc1 ; c1ccc(-c2cc(-c3ccccc3)cc(-c3cc(-c4ccc5c(c4)sc4ccccc45)nc(-c4ccc5c(c4)sc4ccccc45)n3)c2)cc1 ; c1ccc(-c2cc(-c3ccc4ccccc4c3)cc(-c3cc(-c4ccc5c(c4)sc4ccccc45)nc(-c4ccc5c(c4)sc4ccccc45)n3)c2)cc1]

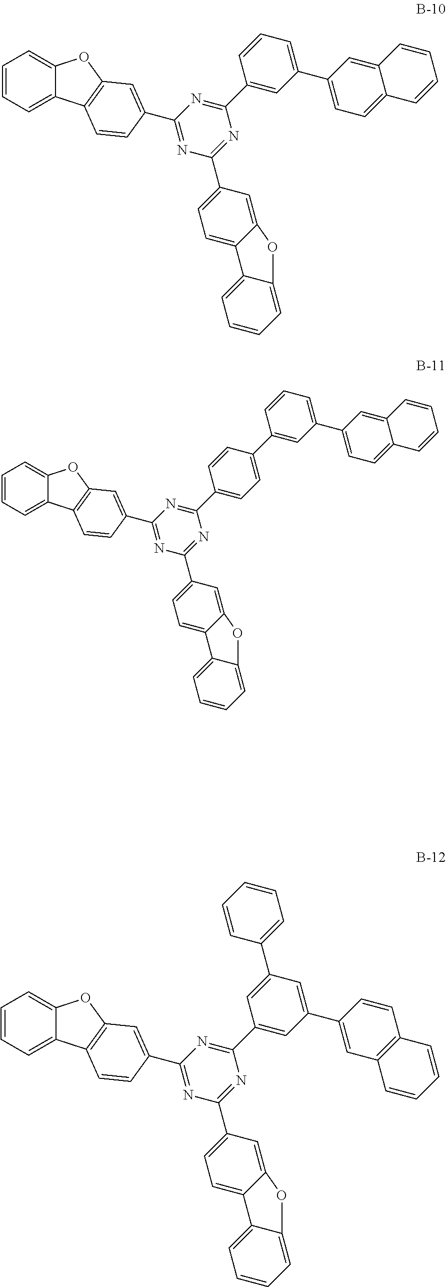 [SMILES: c1cc(-c2ccc3ccccc3c2)cc(-c2nc(-c3ccc4c(c3)oc3ccccc34)nc(-c3ccc4c(c3)oc3ccccc34)n2)c1 ; c1cc(-c2ccc(-c3nc(-c4ccc5c(c4)oc4ccccc45)nc(-c4ccc5c(c4)oc4ccccc45)n3)cc2)cc(-c2ccc3ccccc3c2)c1 ; c1ccc(-c2cc(-c3ccc4ccccc4c3)cc(-c3nc(-c4ccc5c(c4)oc4ccccc45)nc(-c4ccc5c(c4)oc4ccccc45)n3)c2)cc1]

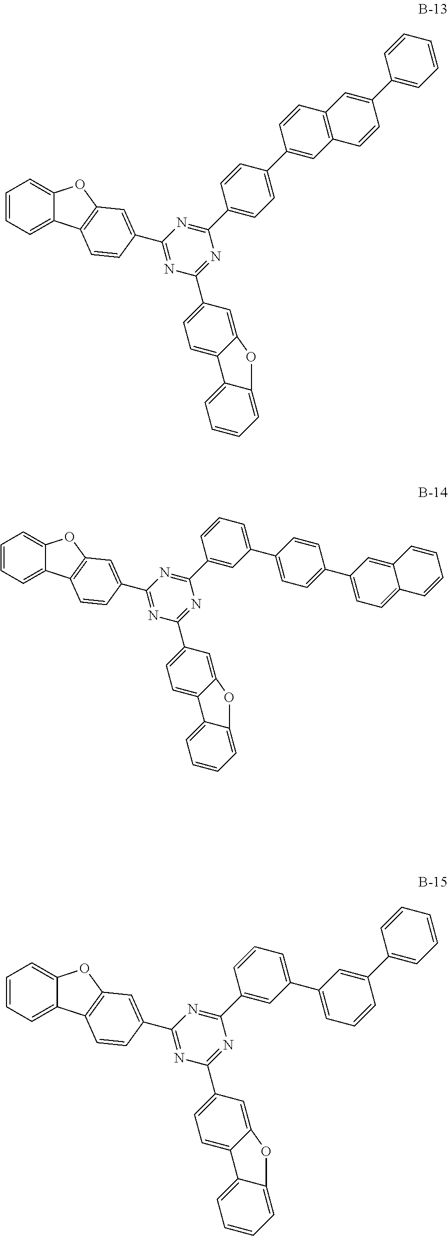 [SMILES: c1ccc(-c2ccc3cc(-c4ccc(-c5nc(-c6ccc7c(c6)oc6ccccc67)nc(-c6ccc7c(c6)oc6ccccc67)n5)cc4)ccc3c2)cc1 ; c1cc(-c2ccc(-c3ccc4ccccc4c3)cc2)cc(-c2nc(-c3ccc4c(c3)oc3ccccc34)nc(-c3ccc4c(c3)oc3ccccc34)n2)c1 ; c1ccc(-c2cccc(-c3cccc(-c4nc(-c5ccc6c(c5)oc5ccccc56)nc(-c5ccc6c(c5)oc5ccccc56)n4)c3)c2)cc1]

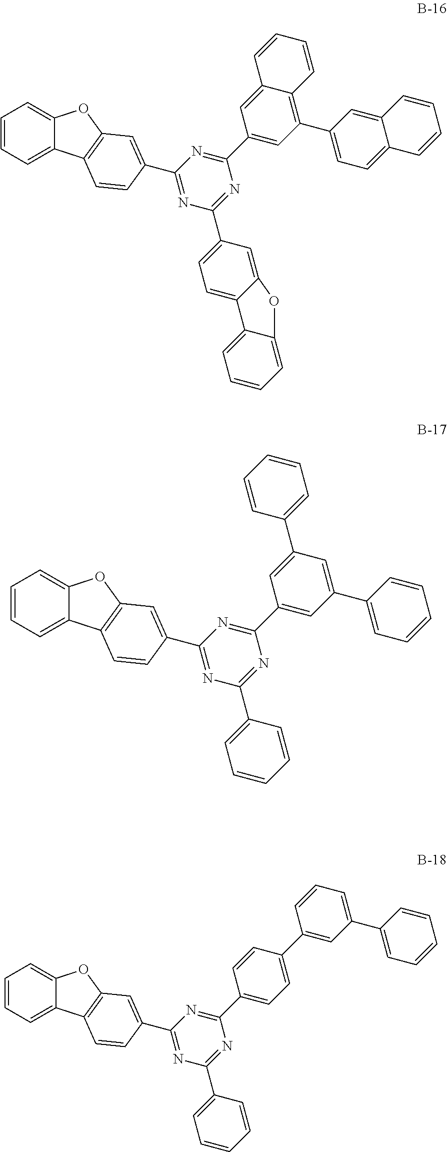 [SMILES: c1ccc2cc(-c3cc(-c4nc(-c5ccc6c(c5)oc5ccccc56)nc(-c5ccc6c(c5)oc5ccccc56)n4)cc4ccccc34)ccc2c1 ; c1ccc(-c2cc(-c3ccccc3)cc(-c3nc(-c4ccccc4)nc(-c4ccc5c(c4)oc4ccccc45)n3)c2)cc1 ; c1ccc(-c2cccc(-c3ccc(-c4nc(-c5ccccc5)nc(-c5ccc6c(c5)oc5ccccc56)n4)cc3)c2)cc1]

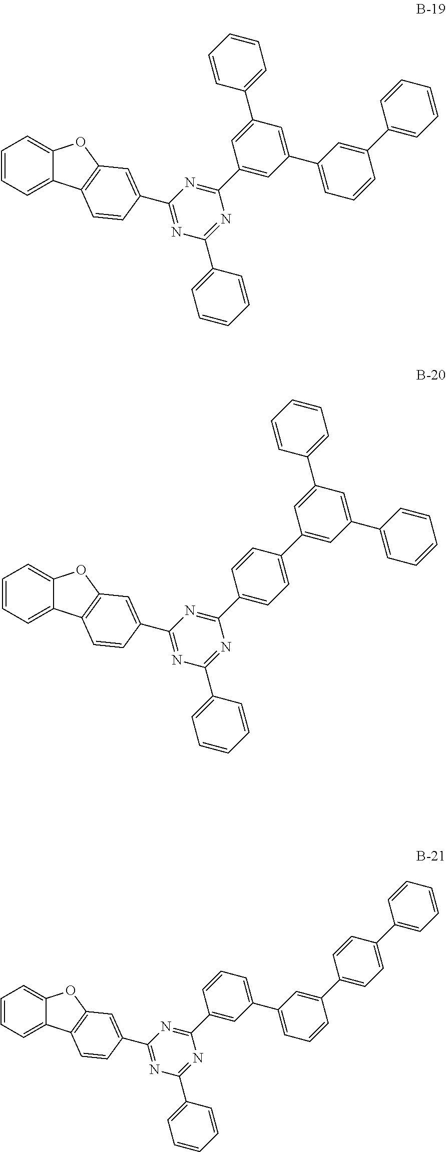 [SMILES: c1ccc(-c2cccc(-c3cc(-c4ccccc4)cc(-c4nc(-c5ccccc5)nc(-c5ccc6c(c5)oc5ccccc56)n4)c3)c2)cc1 ; c1ccc(-c2cc(-c3ccccc3)cc(-c3ccc(-c4nc(-c5ccccc5)nc(-c5ccc6c(c5)oc5ccccc56)n4)cc3)c2)cc1 ; c1ccc(-c2ccc(-c3cccc(-c4cccc(-c5nc(-c6ccccc6)nc(-c6ccc7c(c6)oc6ccccc67)n5)c4)c3)cc2)cc1]

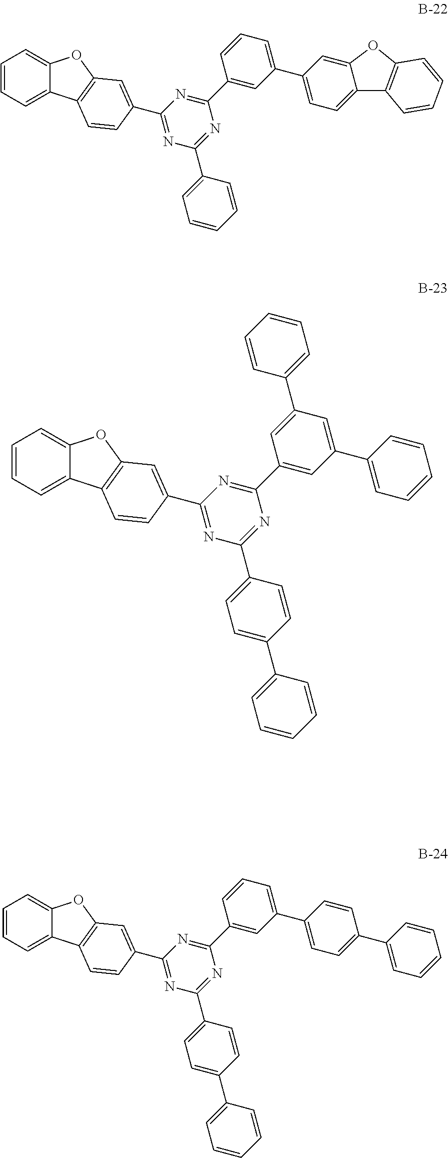 [SMILES: c1ccc(-c2nc(-c3cccc(-c4ccc5c(c4)oc4ccccc45)c3)nc(-c3ccc4c(c3)oc3ccccc34)n2)cc1 ; c1ccc(-c2ccc(-c3nc(-c4cc(-c5ccccc5)cc(-c5ccccc5)c4)nc(-c4ccc5c(c4)oc4ccccc45)n3)cc2)cc1 ; c1ccc(-c2ccc(-c3cccc(-c4nc(-c5ccc(-c6ccccc6)cc5)nc(-c5ccc6c(c5)oc5ccccc56)n4)c3)cc2)cc1]

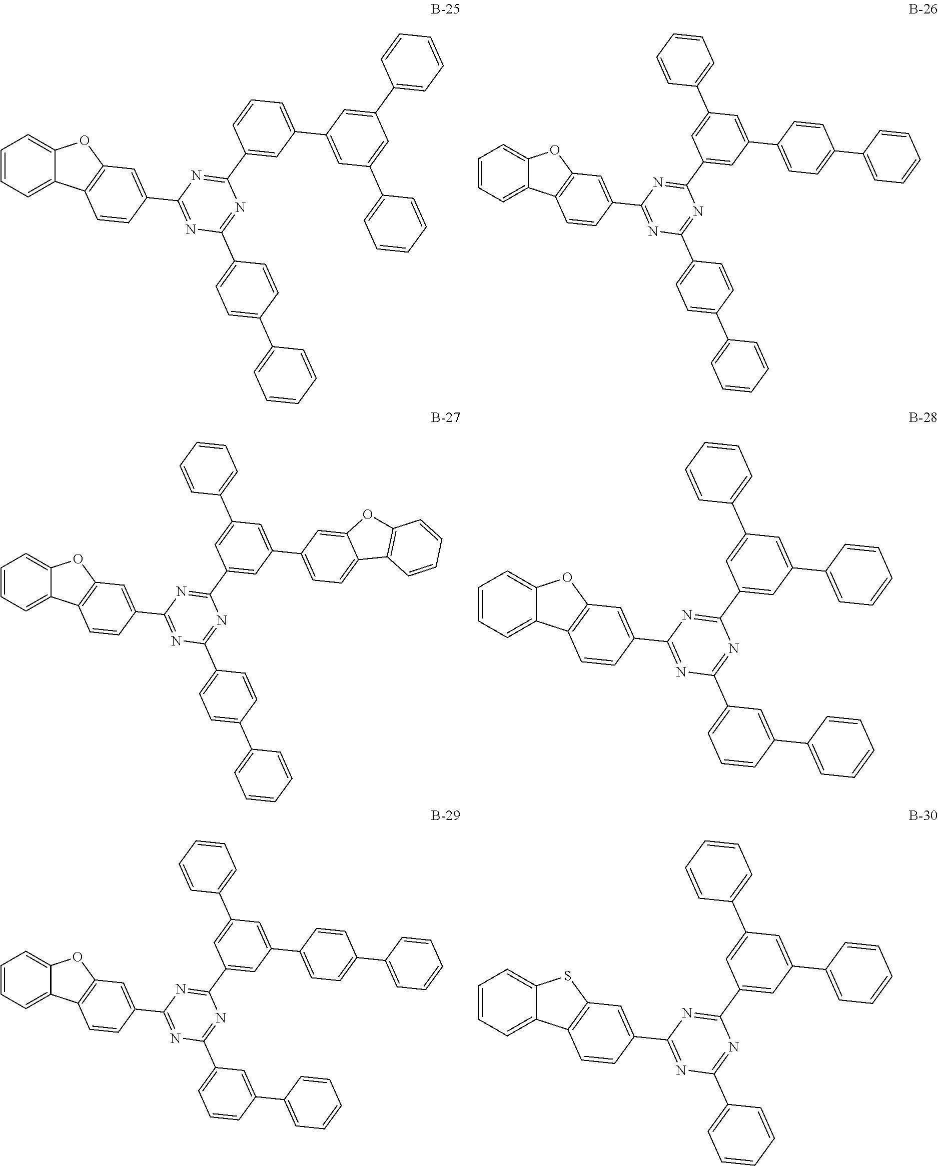

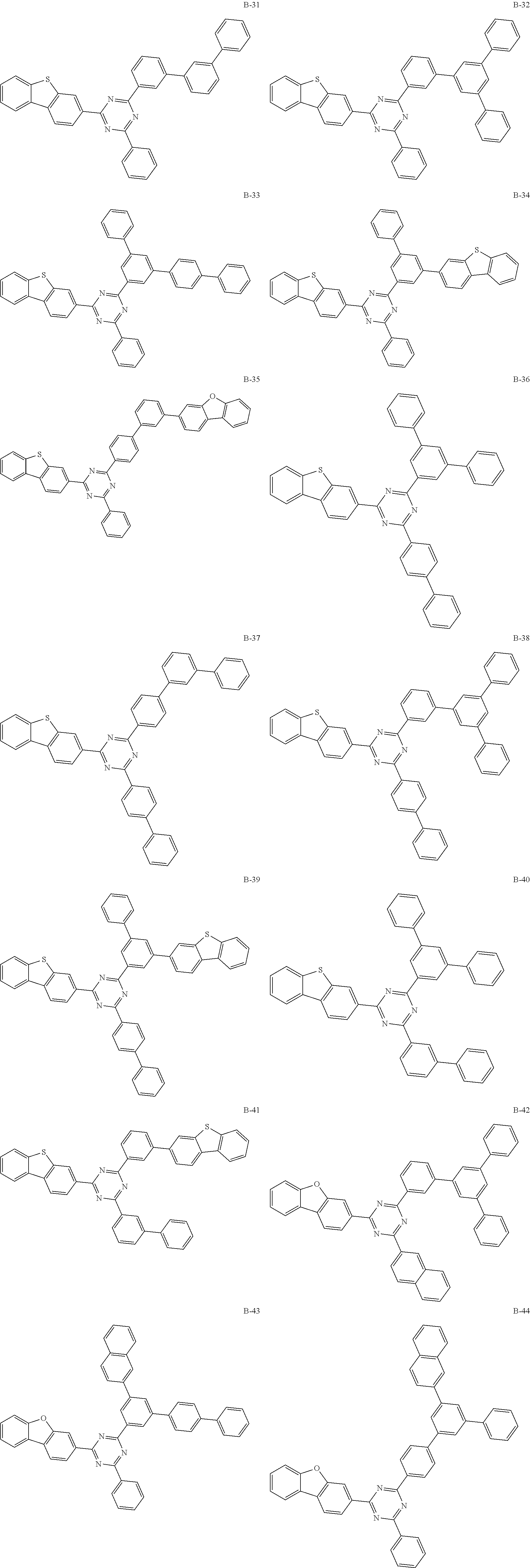

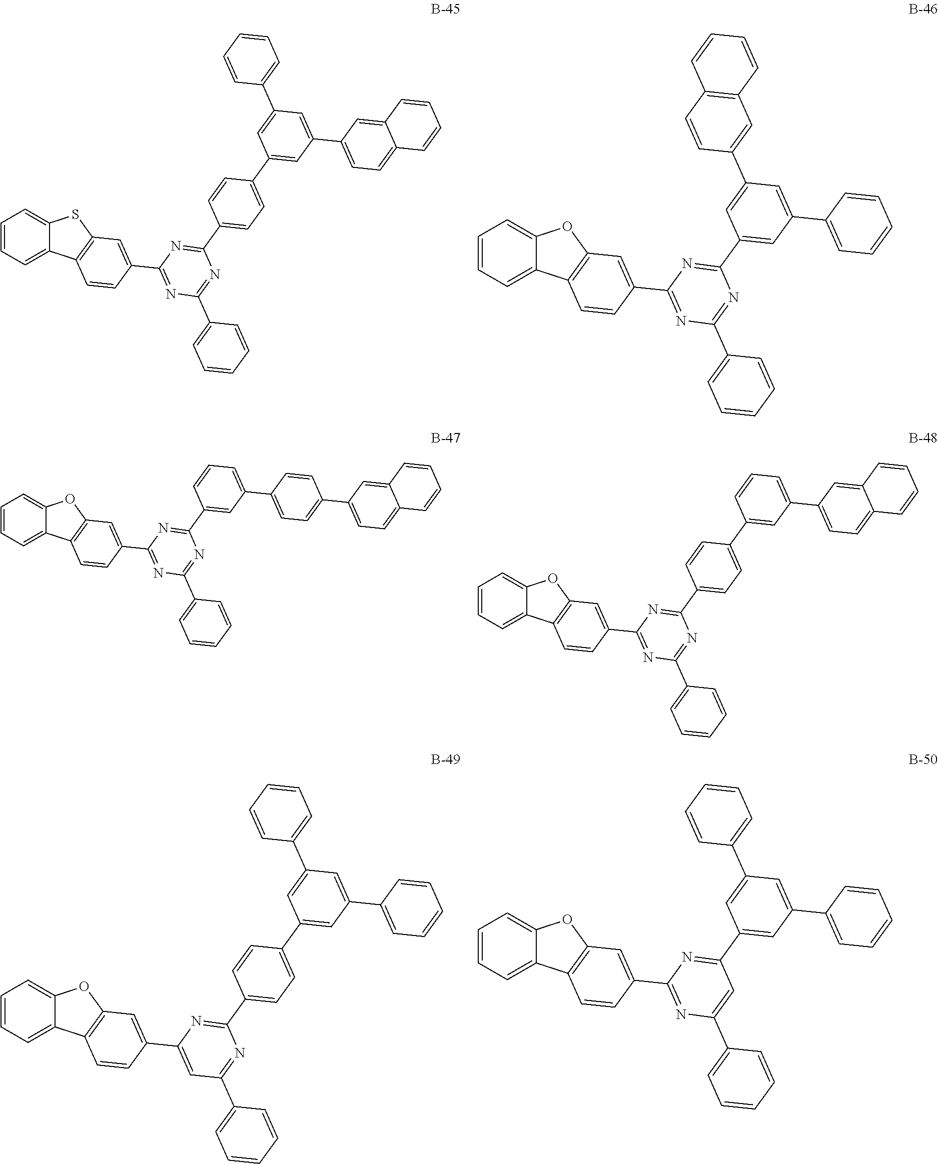

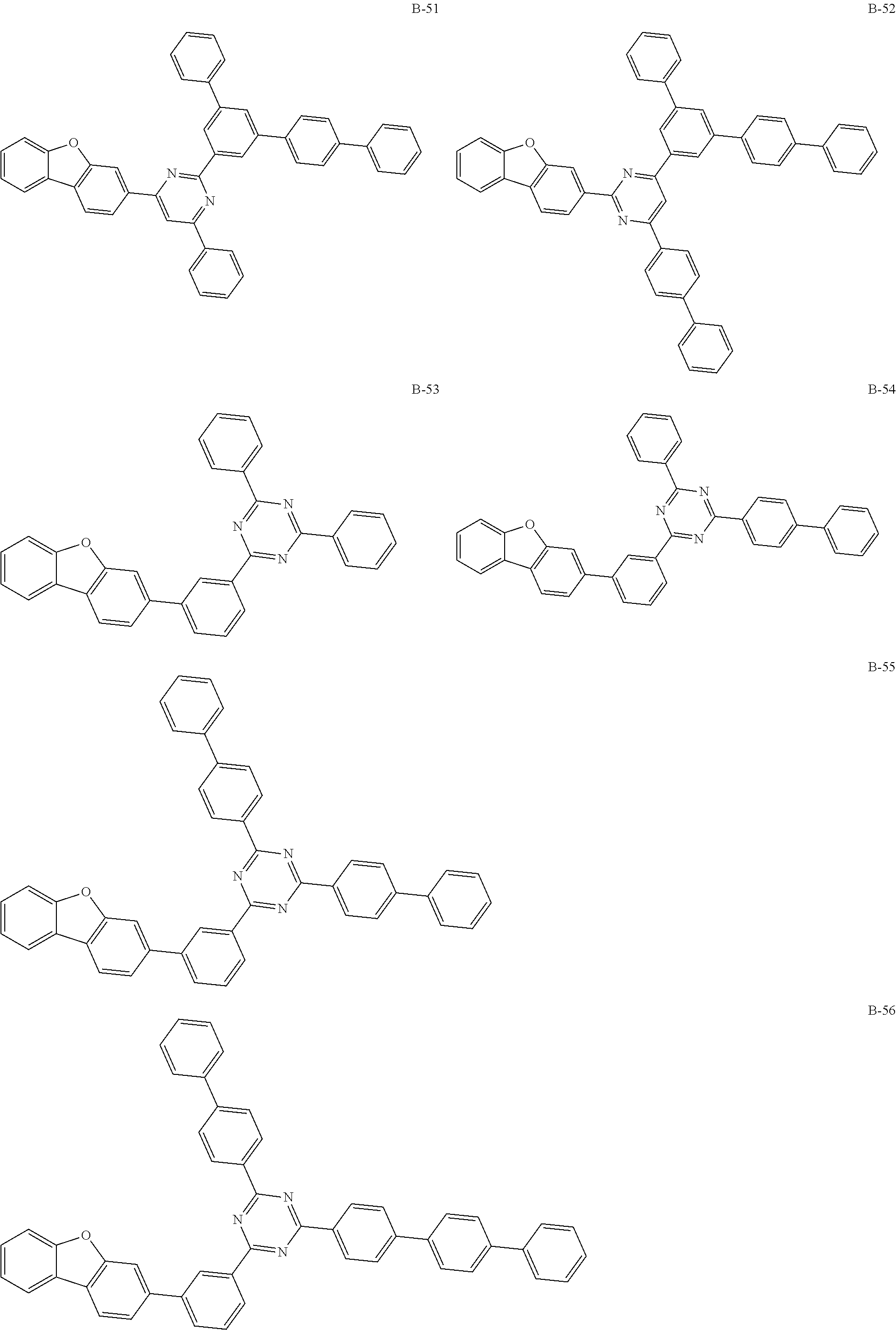

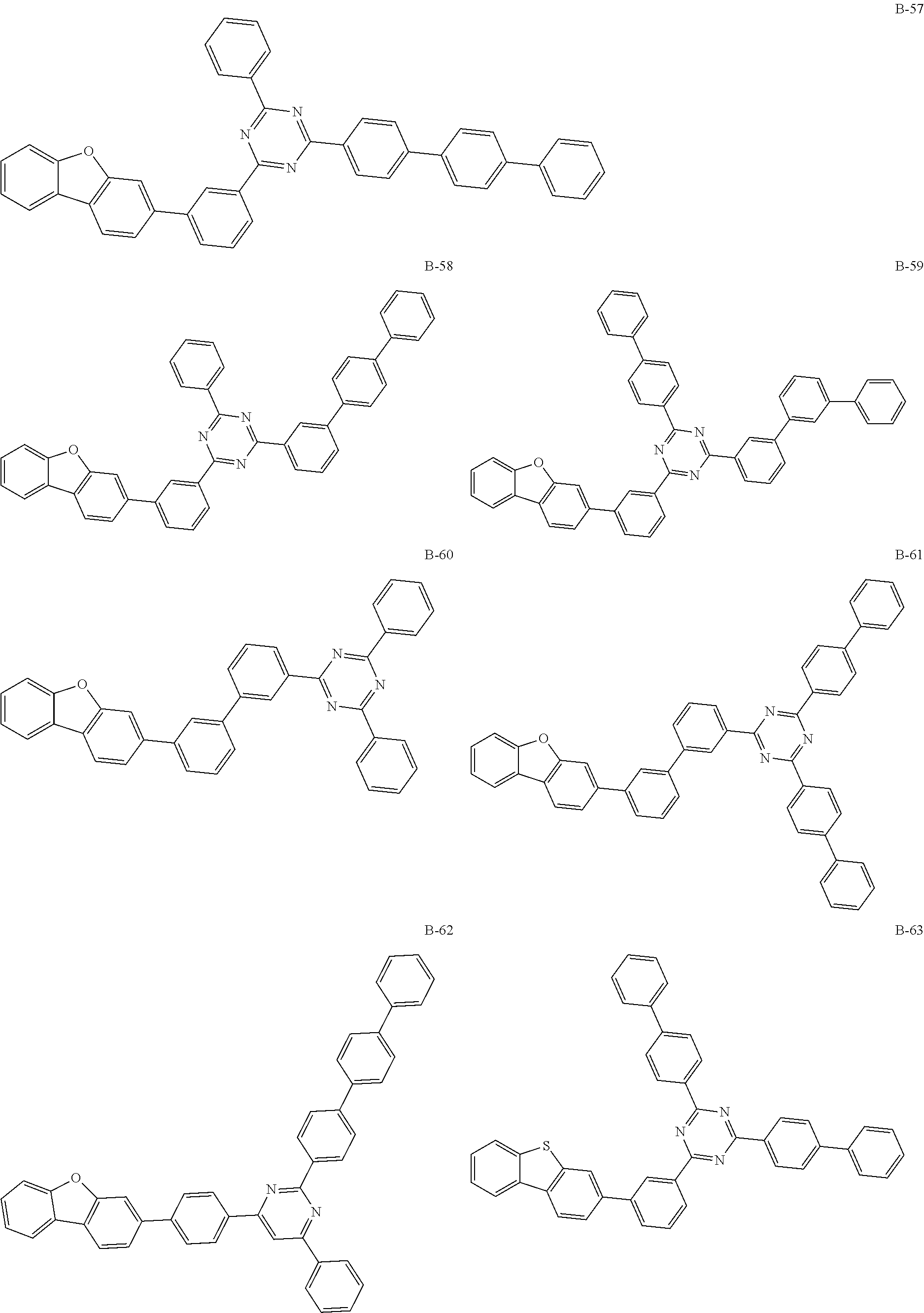

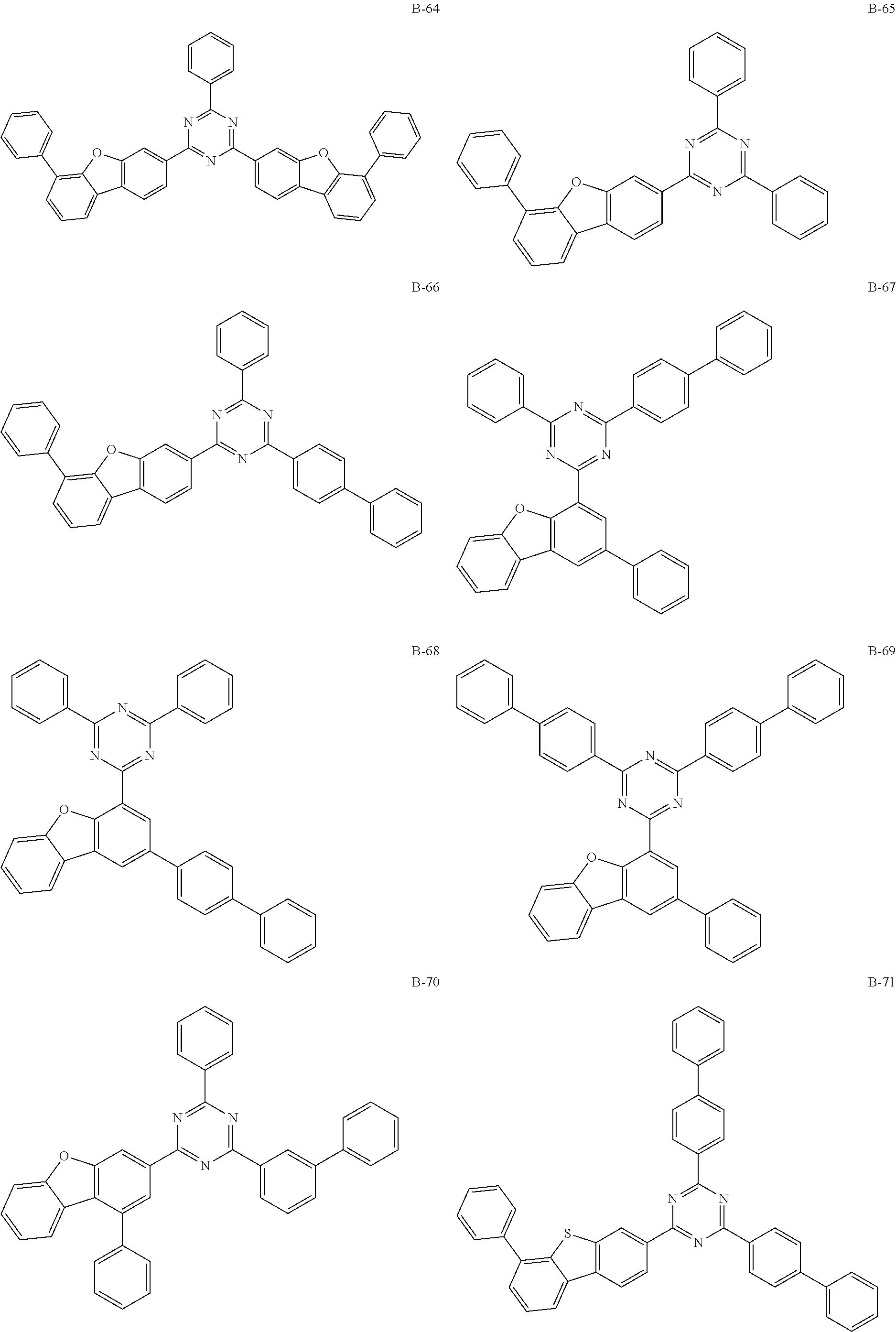

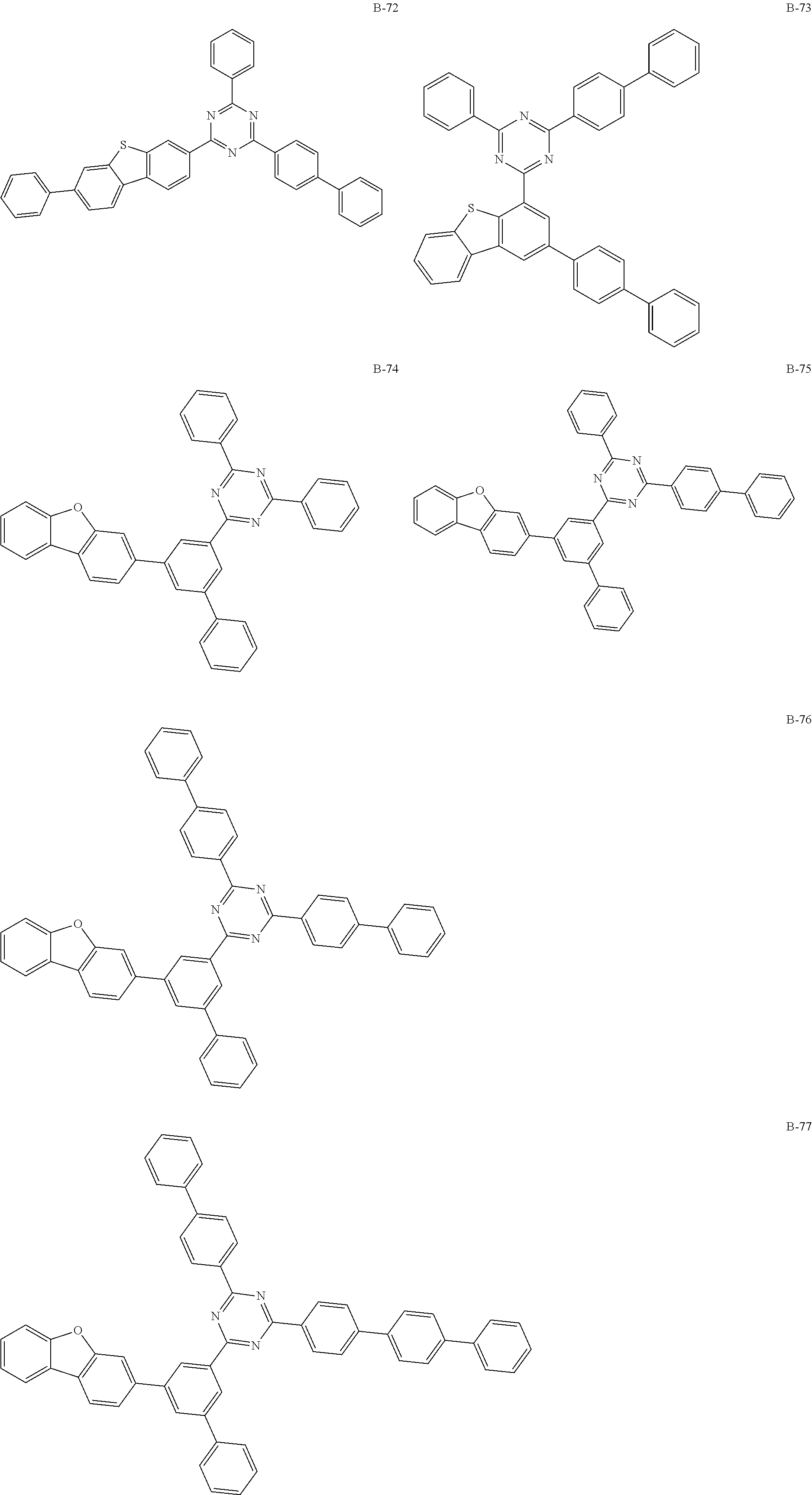

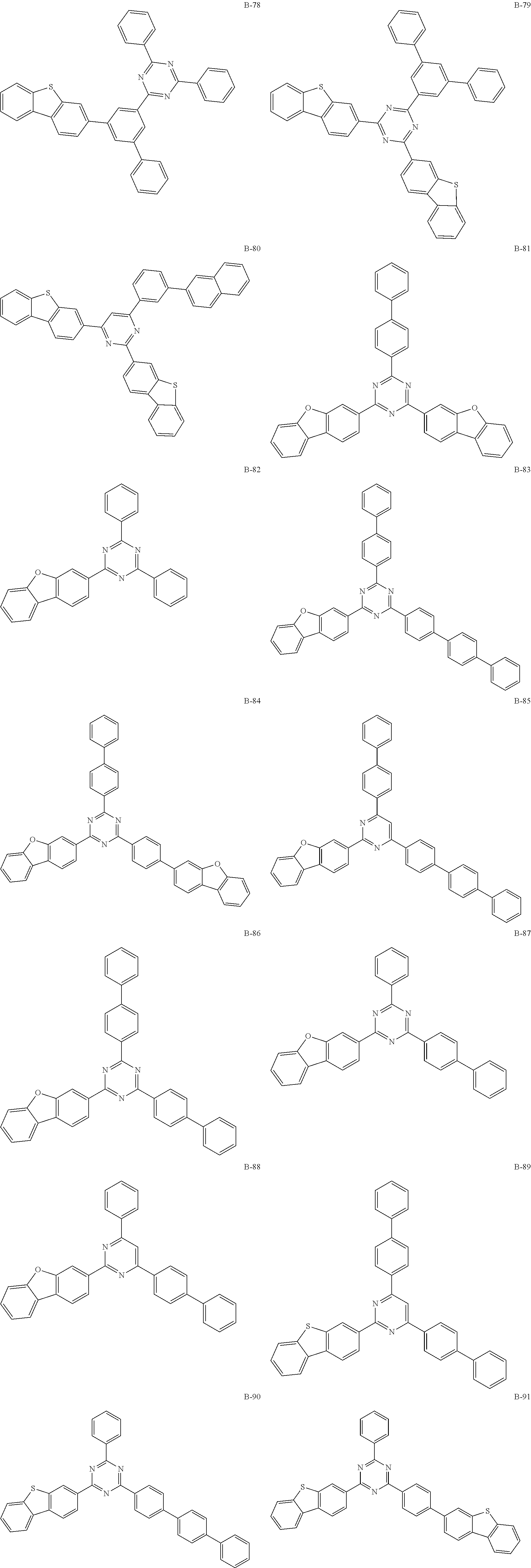

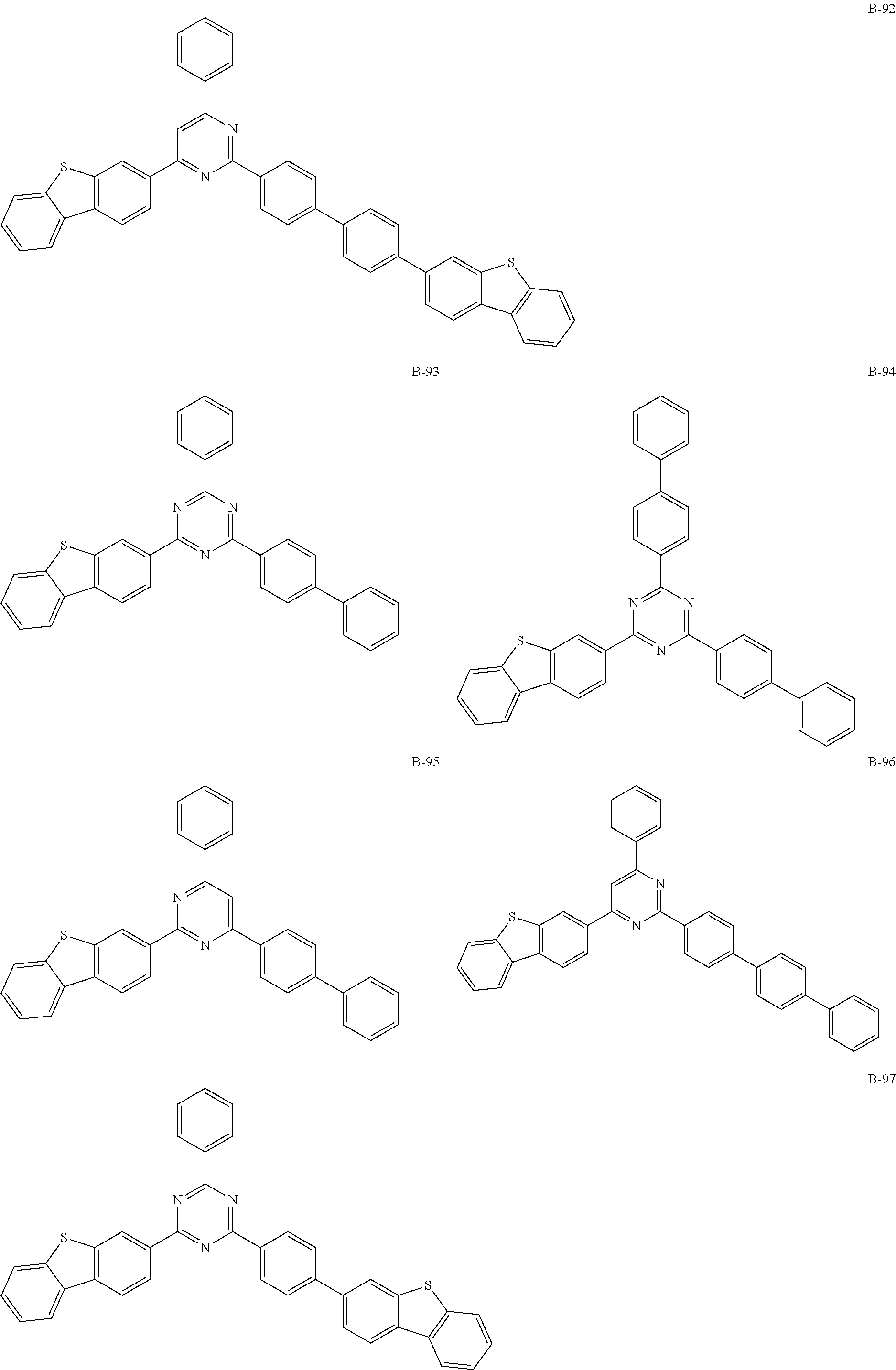 [SMILES: c1ccc(-c2cc(-c3ccc4c(c3)sc3ccccc34)nc(-c3ccc(-c4ccc(-c5ccc6c(c5)sc5ccccc56)cc4)cc3)n2)cc1 ; c1ccc(-c2ccc(-c3nc(-c4ccccc4)nc(-c4ccc5c(c4)sc4ccccc45)n3)cc2)cc1 ; c1ccc(-c2ccc(-c3nc(-c4ccc(-c5ccccc5)cc4)nc(-c4ccc5c(c4)sc4ccccc45)n3)cc2)cc1 ; c1ccc(-c2ccc(-c3cc(-c4ccccc4)nc(-c4ccc5c(c4)sc4ccccc45)n3)cc2)cc1 ; c1ccc(-c2ccc(-c3ccc(-c4nc(-c5ccccc5)cc(-c5ccc6c(c5)sc5ccccc56)n4)cc3)cc2)cc1 ; c1ccc(-c2nc(-c3ccc(-c4ccc5c(c4)sc4ccccc45)cc3)nc(-c3ccc4c(c3)sc3ccccc34)n2)cc1]

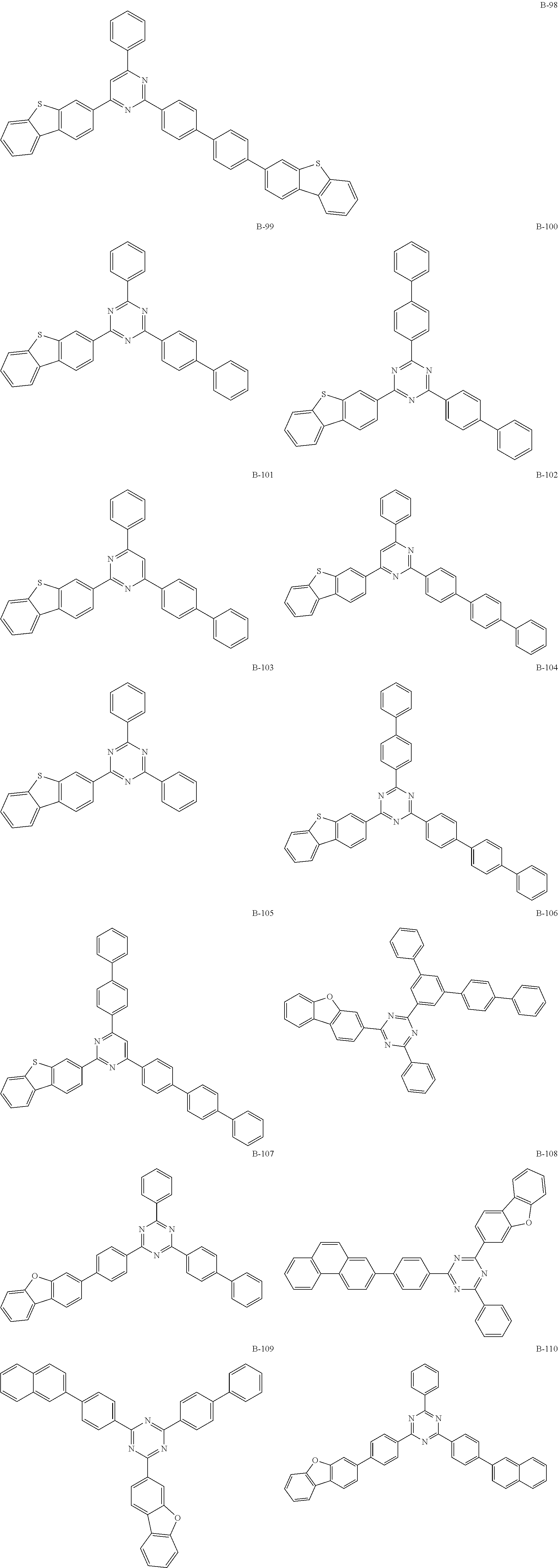

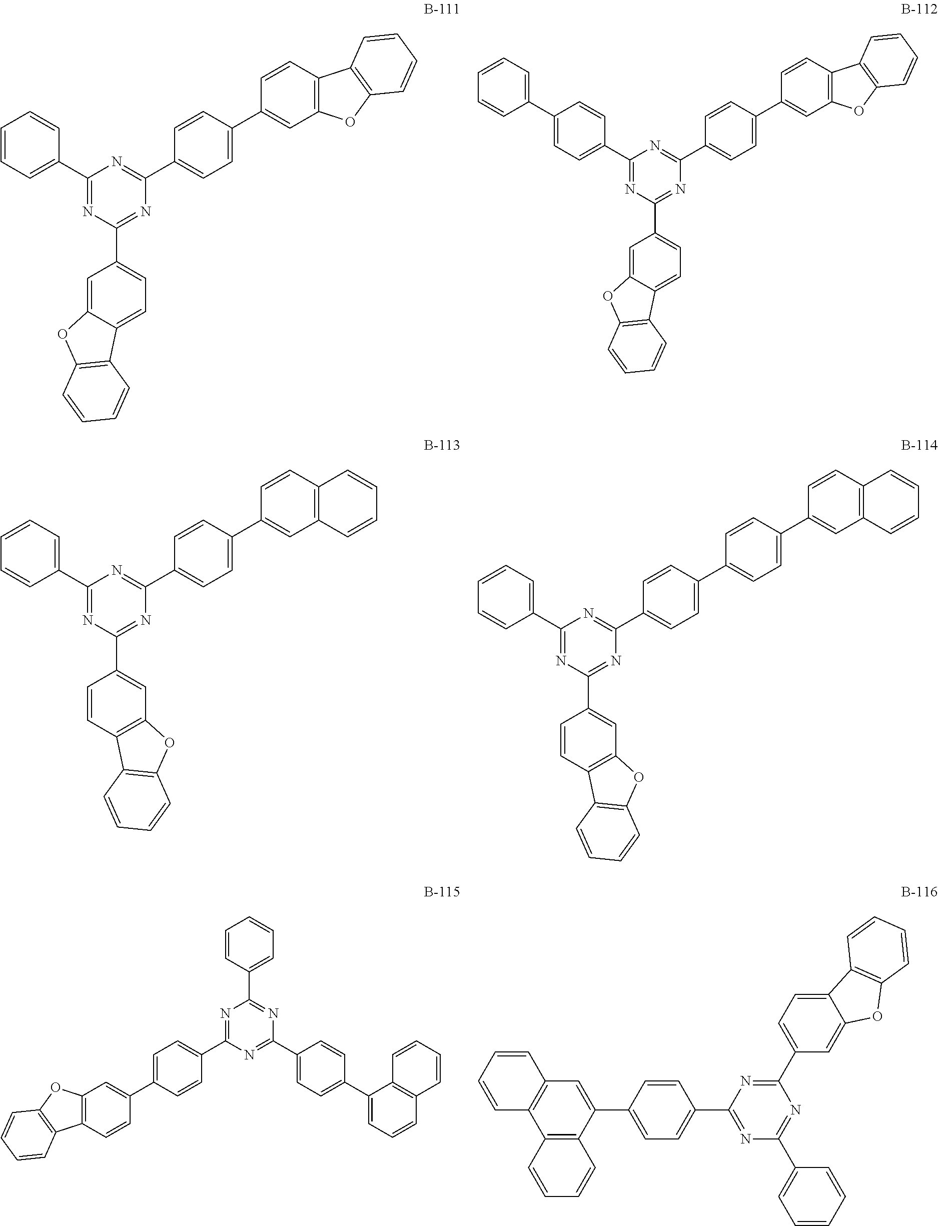

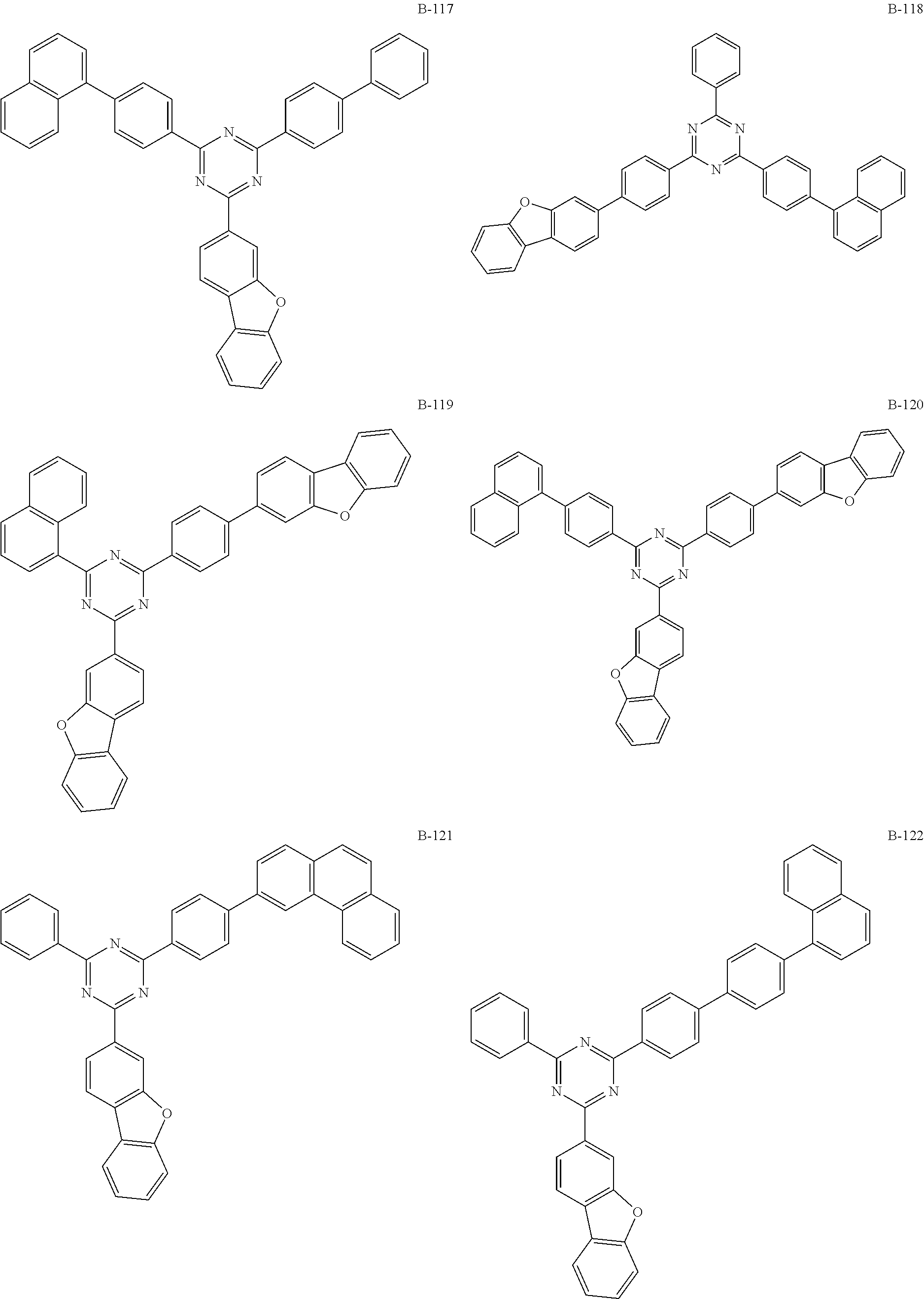

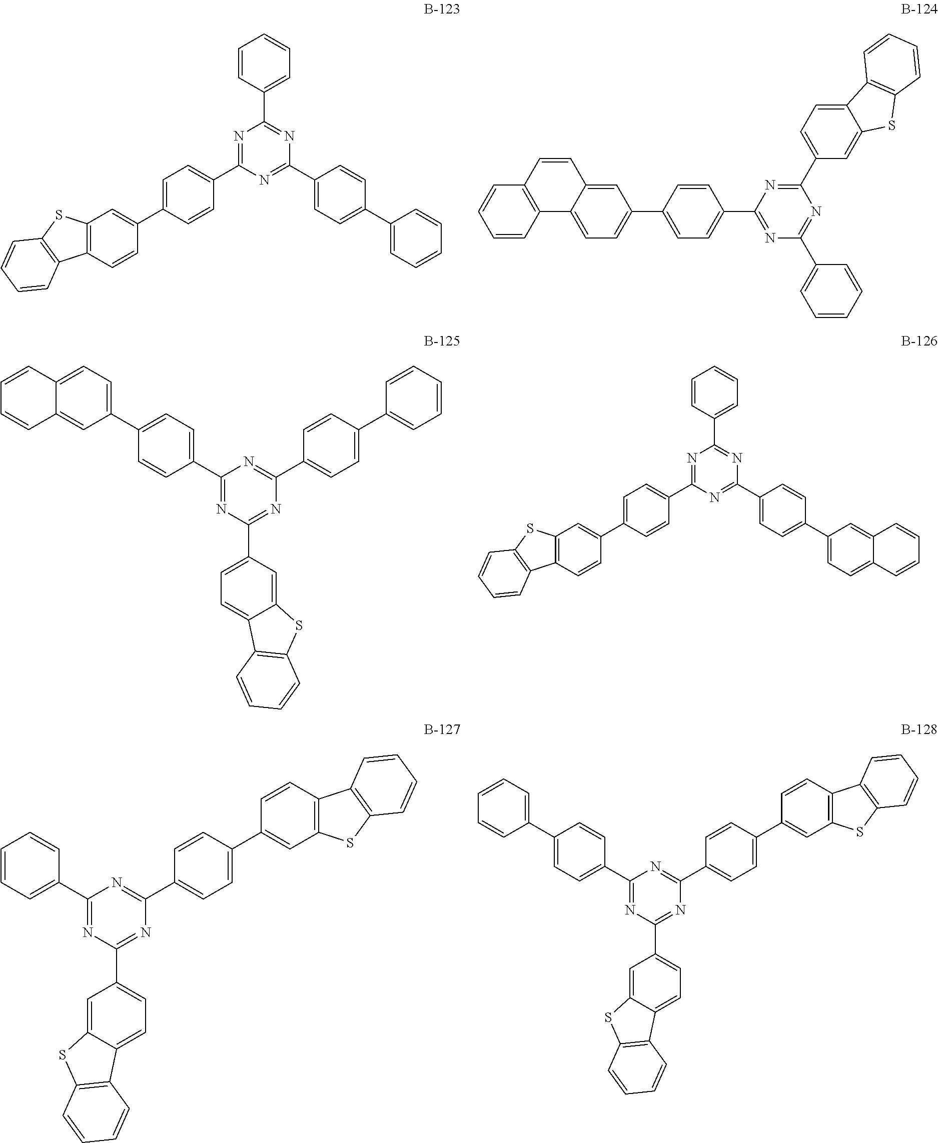

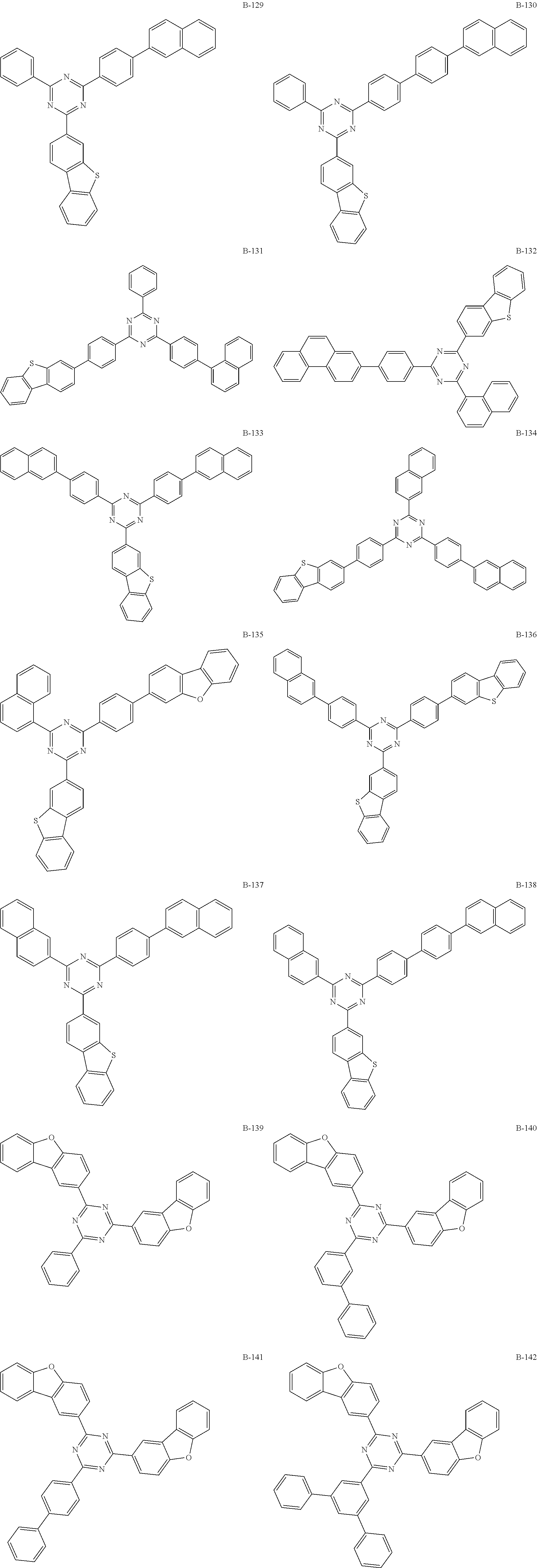

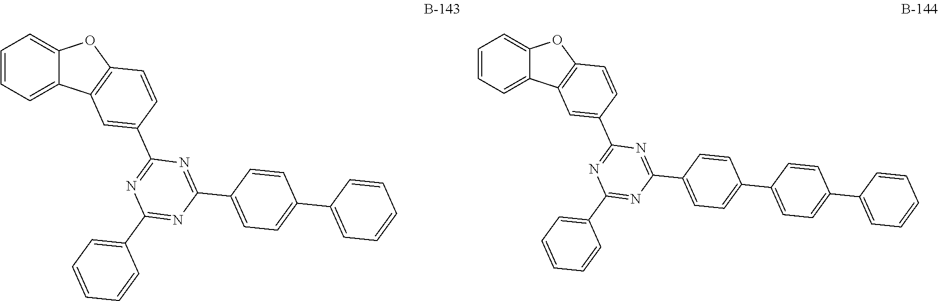 [SMILES: c1ccc(-c2ccc(-c3nc(-c4ccccc4)nc(-c4ccc5oc6ccccc6c5c4)n3)cc2)cc1 ; c1ccc(-c2ccc(-c3ccc(-c4nc(-c5ccccc5)nc(-c5ccc6oc7ccccc7c6c5)n4)cc3)cc2)cc1]

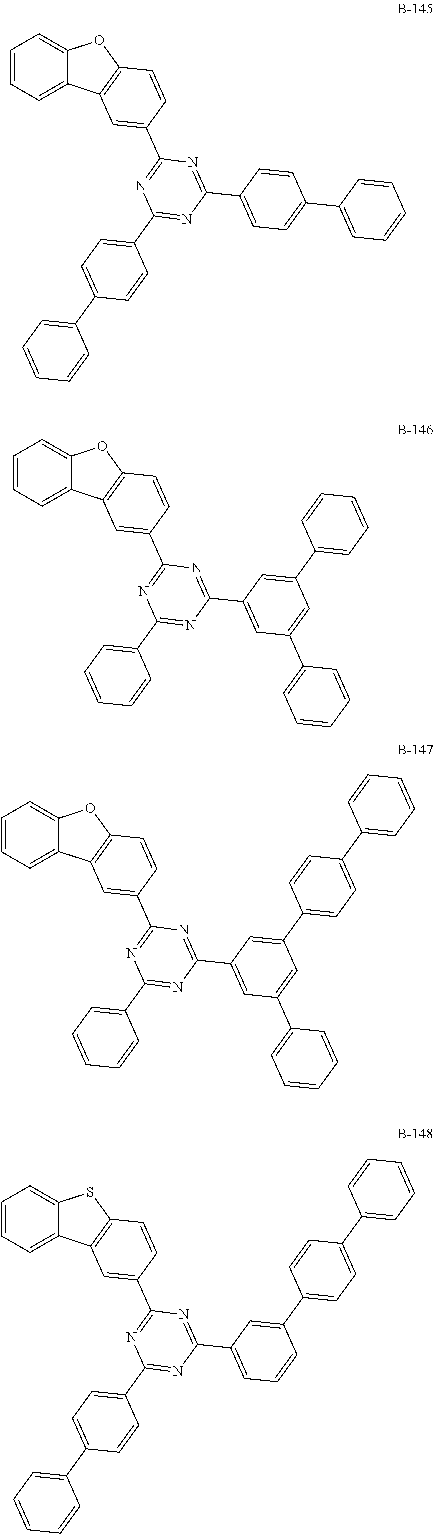 [SMILES: c1ccc(-c2ccc(-c3nc(-c4ccc(-c5ccccc5)cc4)nc(-c4ccc5oc6ccccc6c5c4)n3)cc2)cc1 ; c1ccc(-c2cc(-c3ccccc3)cc(-c3nc(-c4ccccc4)nc(-c4ccc5oc6ccccc6c5c4)n3)c2)cc1 ; c1ccc(-c2ccc(-c3cc(-c4ccccc4)cc(-c4nc(-c5ccccc5)nc(-c5ccc6oc7ccccc7c6c5)n4)c3)cc2)cc1 ; c1ccc(-c2ccc(-c3cccc(-c4nc(-c5ccc(-c6ccccc6)cc5)nc(-c5ccc6sc7ccccc7c6c5)n4)c3)cc2)cc1]

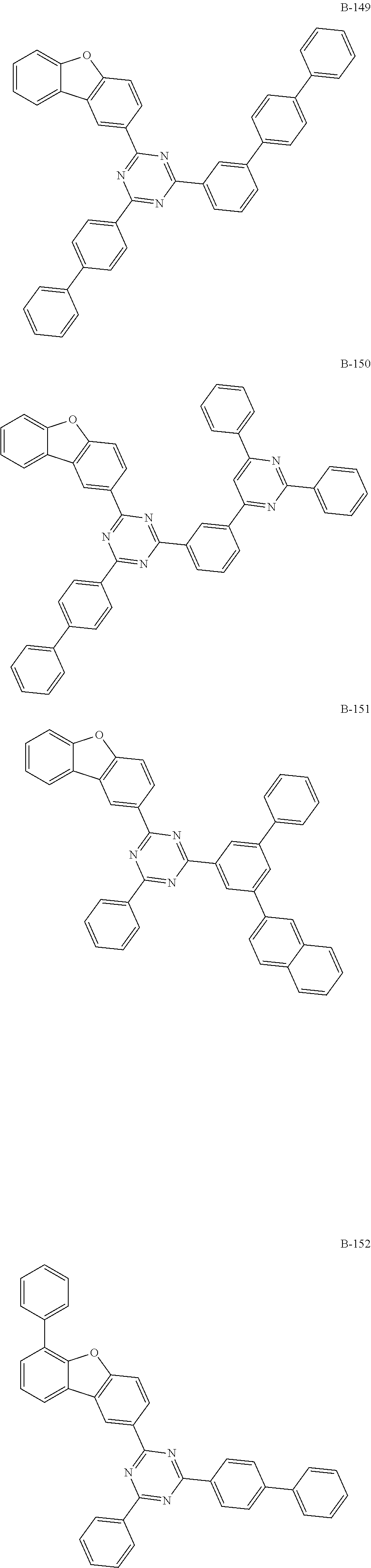

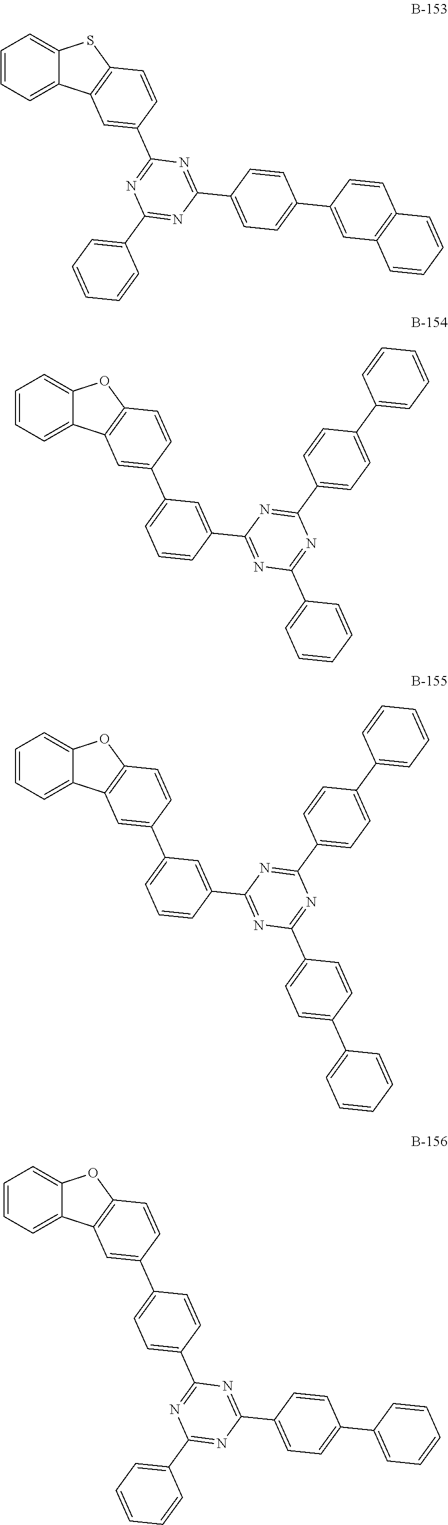 [SMILES: c1ccc(-c2nc(-c3ccc(-c4ccc5ccccc5c4)cc3)nc(-c3ccc4sc5ccccc5c4c3)n2)cc1 ; c1ccc(-c2ccc(-c3nc(-c4ccccc4)nc(-c4cccc(-c5ccc6oc7ccccc7c6c5)c4)n3)cc2)cc1 ; c1ccc(-c2ccc(-c3nc(-c4ccc(-c5ccccc5)cc4)nc(-c4cccc(-c5ccc6oc7ccccc7c6c5)c4)n3)cc2)cc1 ; c1ccc(-c2ccc(-c3nc(-c4ccccc4)nc(-c4ccc(-c5ccc6oc7ccccc7c6c5)cc4)n3)cc2)cc1]

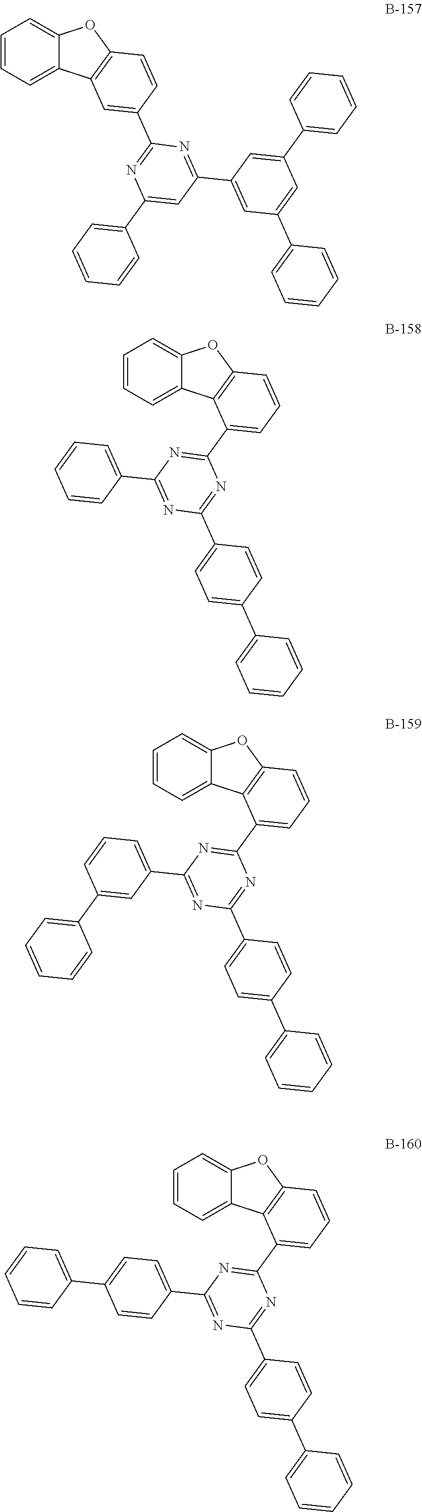 [SMILES: c1ccc(-c2cc(-c3ccccc3)cc(-c3cc(-c4ccccc4)nc(-c4ccc5oc6ccccc6c5c4)n3)c2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1]

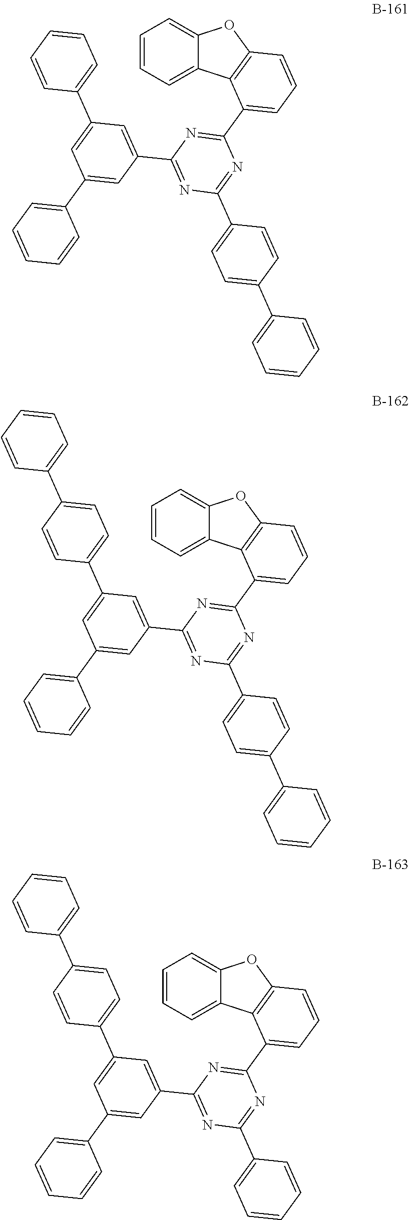 [SMILES: c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cc(-c4ccccc4)cc(-c4nc(-c5ccc(-c6ccccc6)cc5)nc(-c5cccc6oc7ccccc7c56)n4)c3)cc2)cc1 ; c1ccc(-c2ccc(-c3cc(-c4ccccc4)cc(-c4nc(-c5ccccc5)nc(-c5cccc6oc7ccccc7c56)n4)c3)cc2)cc1]

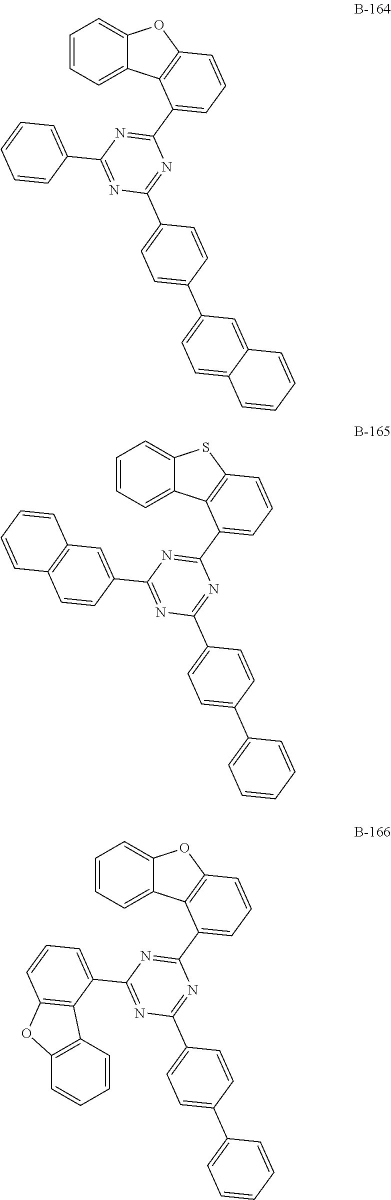 [SMILES: c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc5ccccc5c4)nc(-c4cccc5sc6ccccc6c45)n3)cc2)cc1 ; c1ccc(-c2ccc(-c3nc(-c4cccc5oc6ccccc6c45)nc(-c4cccc5oc6ccccc6c45)n3)cc2)cc1]

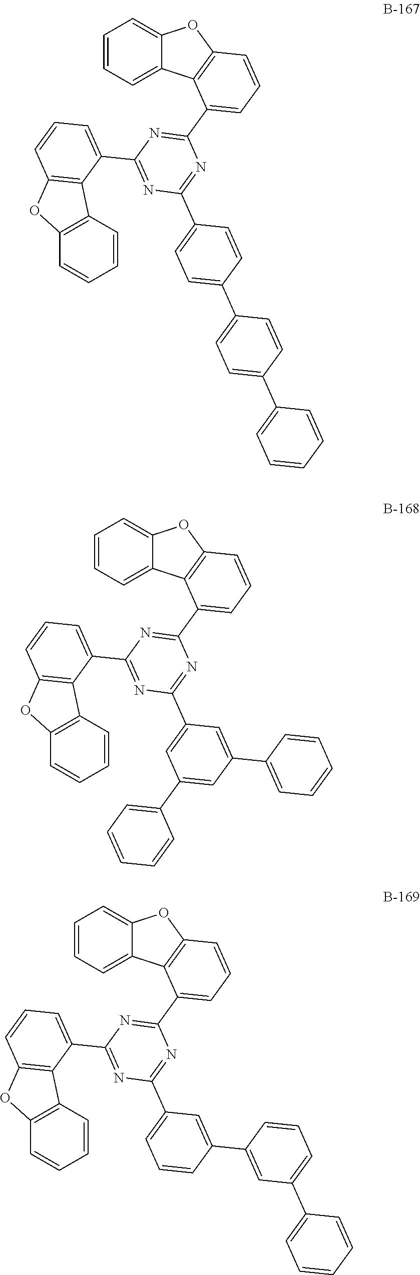 [SMILES: c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1 ; c1ccc(-c2cccc(-c3cccc(-c4nc(-c5cccc6oc7ccccc7c56)nc(-c5cccc6oc7ccccc7c56)n4)c3)c2)cc1]

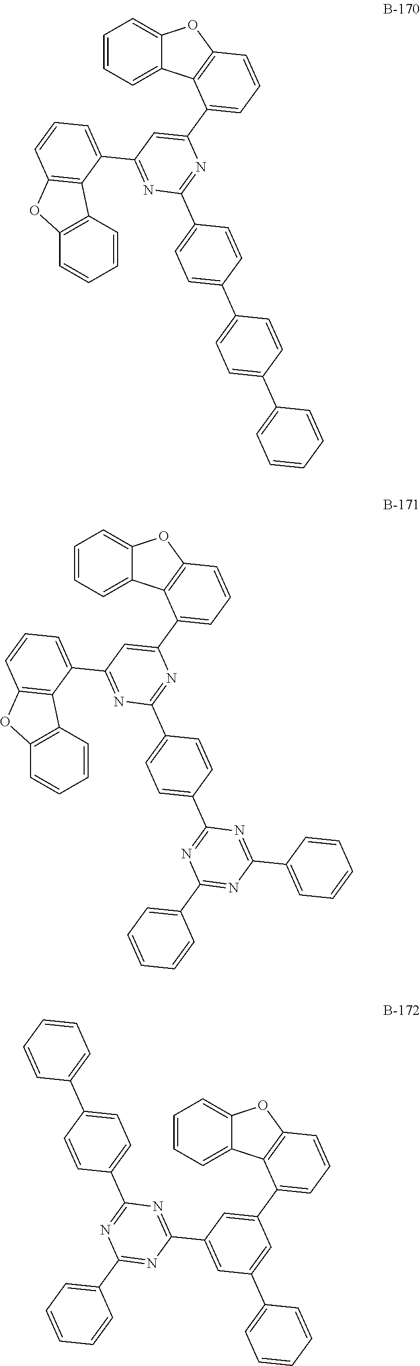 [SMILES: c1ccc(-c2ccc(-c3ccc(-c4nc(-c5cccc6oc7ccccc7c56)cc(-c5cccc6oc7ccccc7c56)n4)cc3)cc2)cc1 ; c1ccc(-c2nc(-c3ccccc3)nc(-c3ccc(-c4nc(-c5cccc6oc7ccccc7c56)cc(-c5cccc6oc7ccccc7c56)n4)cc3)n2)cc1 ; c1ccc(-c2ccc(-c3nc(-c4ccccc4)nc(-c4cc(-c5ccccc5)cc(-c5cccc6oc7ccccc7c56)c4)n3)cc2)cc1]

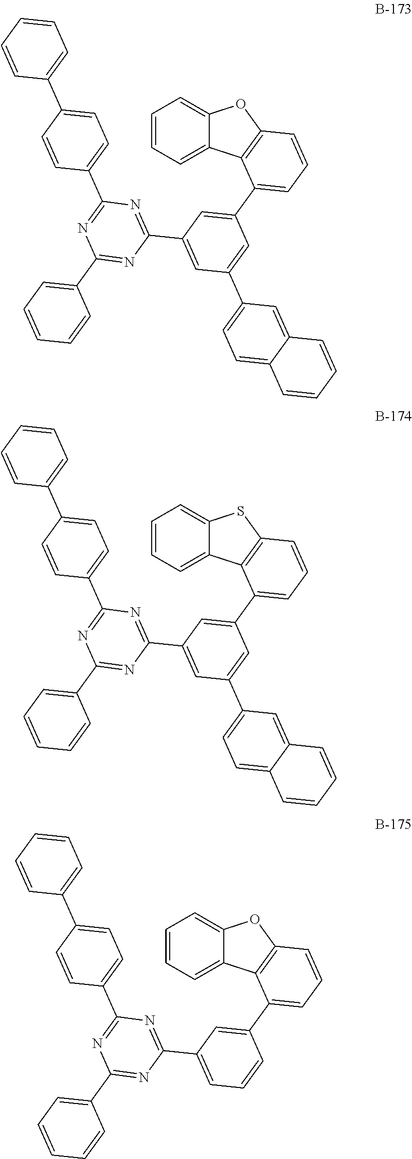 [SMILES: c1ccc(-c2ccc(-c3nc(-c4ccccc4)nc(-c4cc(-c5ccc6ccccc6c5)cc(-c5cccc6oc7ccccc7c56)c4)n3)cc2)cc1 ; c1ccc(-c2ccc(-c3nc(-c4ccccc4)nc(-c4cc(-c5ccc6ccccc6c5)cc(-c5cccc6sc7ccccc7c56)c4)n3)cc2)cc1 ; c1ccc(-c2ccc(-c3nc(-c4ccccc4)nc(-c4cccc(-c5cccc6oc7ccccc7c56)c4)n3)cc2)cc1]

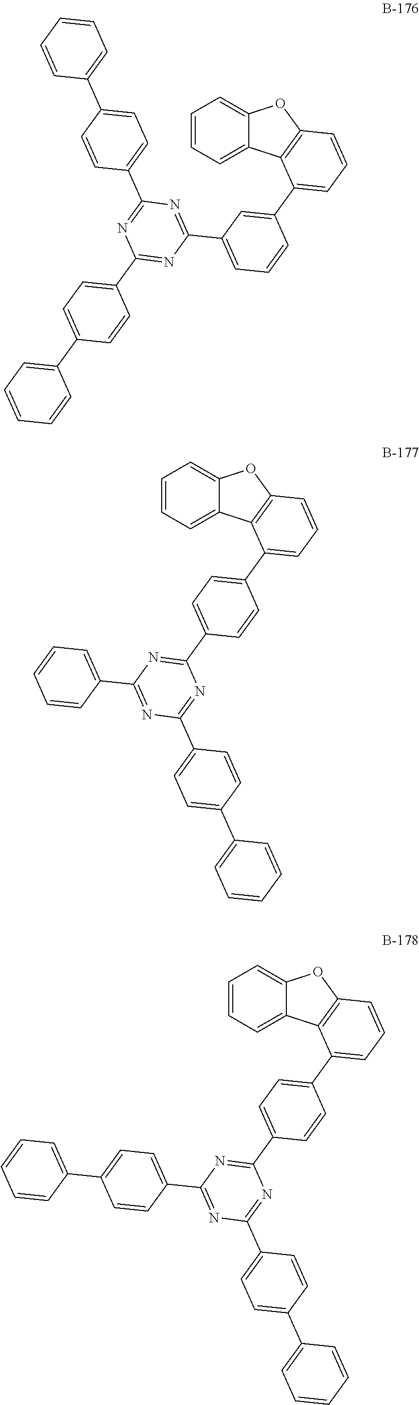 [SMILES: c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

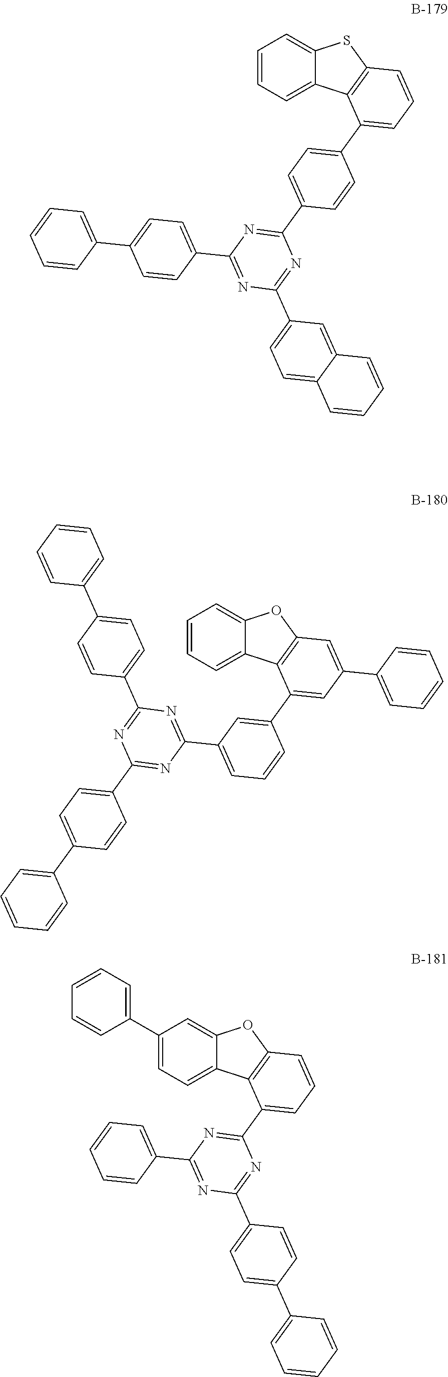 [SMILES: c1ccc(-c2ccc(-c3nc(-c4ccc(-c5cccc6sc7ccccc7c56)cc4)nc(-c4ccc5ccccc5c4)n3)cc2)cc1 ; c1ccc(-c2ccc(-c3nc(-c4ccc(-c5ccccc5)cc4)nc(-c4cccc(-c5cc(-c6ccccc6)cc6oc7ccccc7c56)c4)n3)cc2)cc1 ; c1ccc(-c2ccc(-c3nc(-c4ccccc4)nc(-c4cccc5oc6cc(-c7ccccc7)ccc6c45)n3)cc2)cc1]

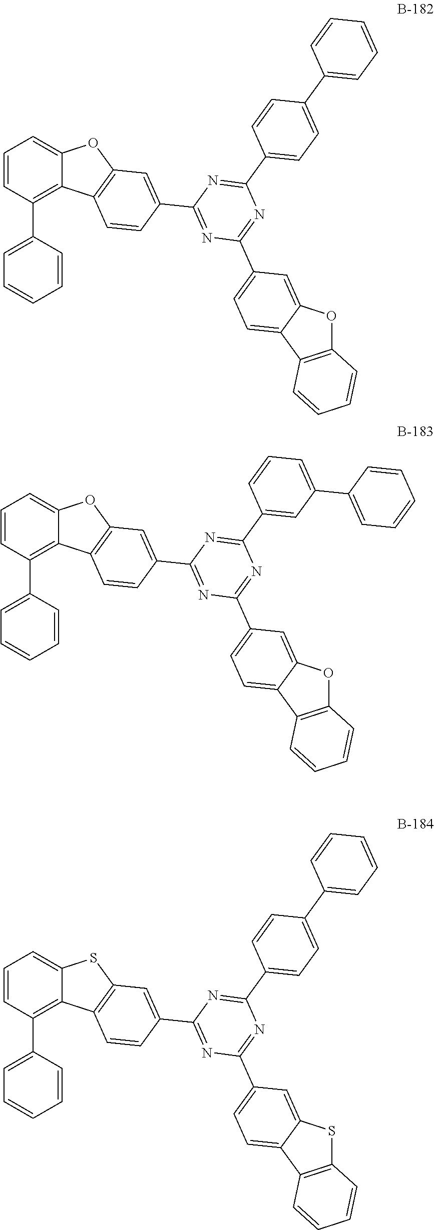 [SMILES: c1ccc(-c2ccc(-c3nc(-c4ccc5c(c4)oc4ccccc45)nc(-c4ccc5c(c4)oc4cccc(-c6ccccc6)c45)n3)cc2)cc1 ; c1ccc(-c2cccc(-c3nc(-c4ccc5c(c4)oc4ccccc45)nc(-c4ccc5c(c4)oc4cccc(-c6ccccc6)c45)n3)c2)cc1 ; c1ccc(-c2ccc(-c3nc(-c4ccc5c(c4)sc4ccccc45)nc(-c4ccc5c(c4)sc4cccc(-c6ccccc6)c45)n3)cc2)cc1]

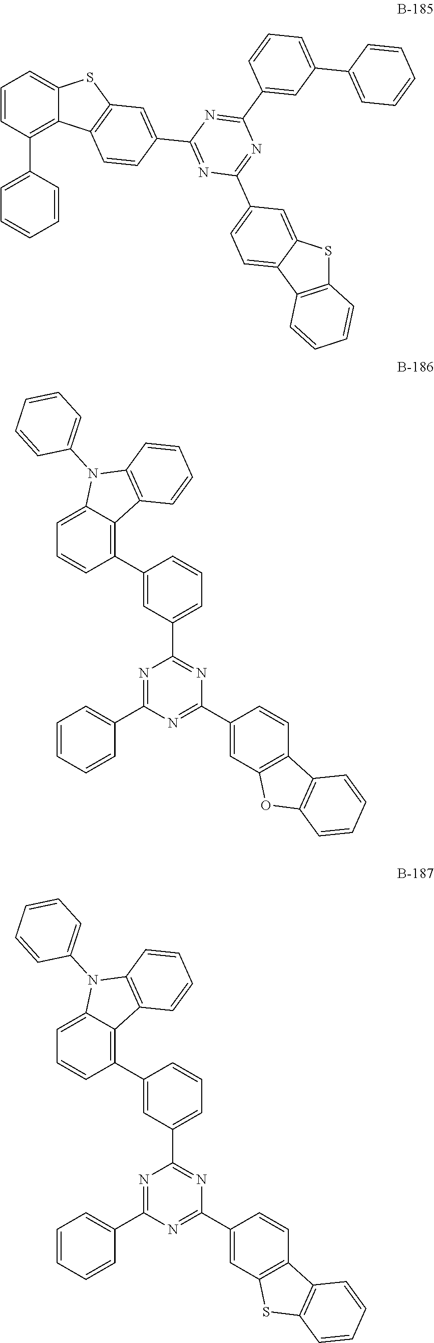 [SMILES: c1ccc(-c2cccc(-c3nc(-c4ccc5c(c4)sc4ccccc45)nc(-c4ccc5c(c4)sc4cccc(-c6ccccc6)c45)n3)c2)cc1 ; c1ccc(-c2nc(-c3cccc(-c4cccc5c4c4ccccc4n5-c4ccccc4)c3)nc(-c3ccc4c(c3)oc3ccccc34)n2)cc1 ; c1ccc(-c2nc(-c3cccc(-c4cccc5c4c4ccccc4n5-c4ccccc4)c3)nc(-c3ccc4c(c3)sc3ccccc34)n2)cc1]

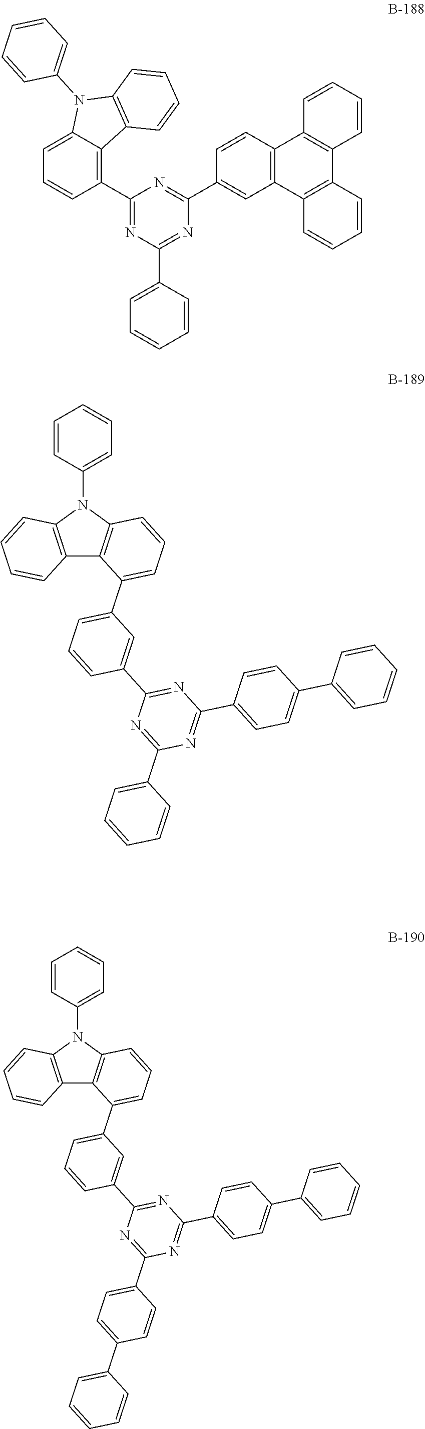 [SMILES: c1ccc(-c2nc(-c3ccc4c5ccccc5c5ccccc5c4c3)nc(-c3cccc4c3c3ccccc3n4-c3ccccc3)n2)cc1 ; c1ccc(-c2ccc(-c3nc(-c4ccccc4)nc(-c4cccc(-c5cccc6c5c5ccccc5n6-c5ccccc5)c4)n3)cc2)cc1 ; c1ccc(-c2ccc(-c3nc(-c4ccc(-c5ccccc5)cc4)nc(-c4cccc(-c5cccc6c5c5ccccc5n6-c5ccccc5)c4)n3)cc2)cc1]

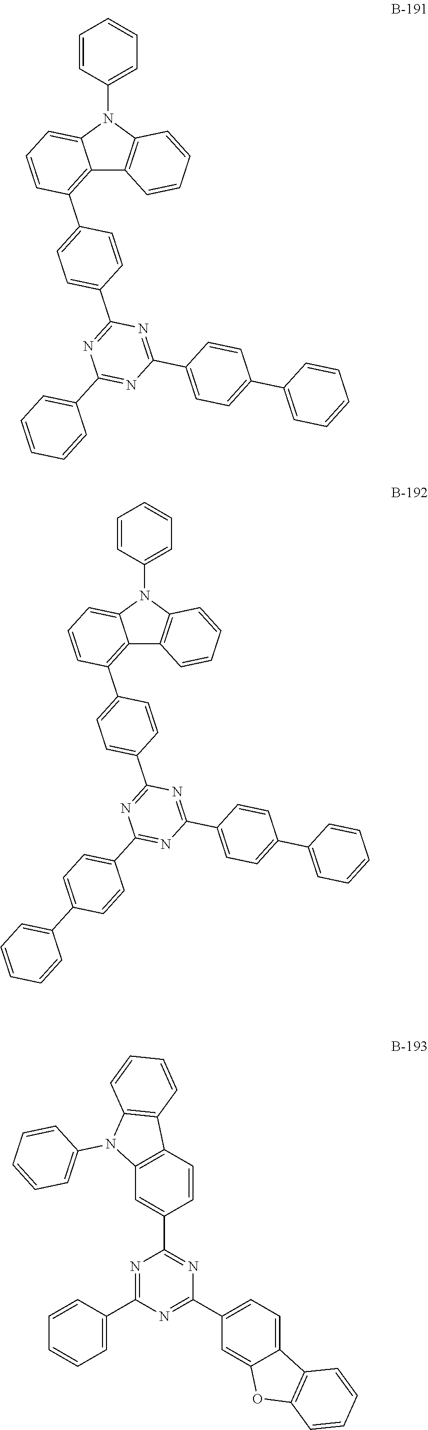 [SMILES: c1ccc(-c2ccc(-c3nc(-c4ccccc4)nc(-c4ccc(-c5cccc6c5c5ccccc5n6-c5ccccc5)cc4)n3)cc2)cc1 ; c1ccc(-c2ccc(-c3nc(-c4ccc(-c5ccccc5)cc4)nc(-c4ccc(-c5cccc6c5c5ccccc5n6-c5ccccc5)cc4)n3)cc2)cc1 ; c1ccc(-c2nc(-c3ccc4c(c3)oc3ccccc34)nc(-c3ccc4c5ccccc5n(-c5ccccc5)c4c3)n2)cc1]

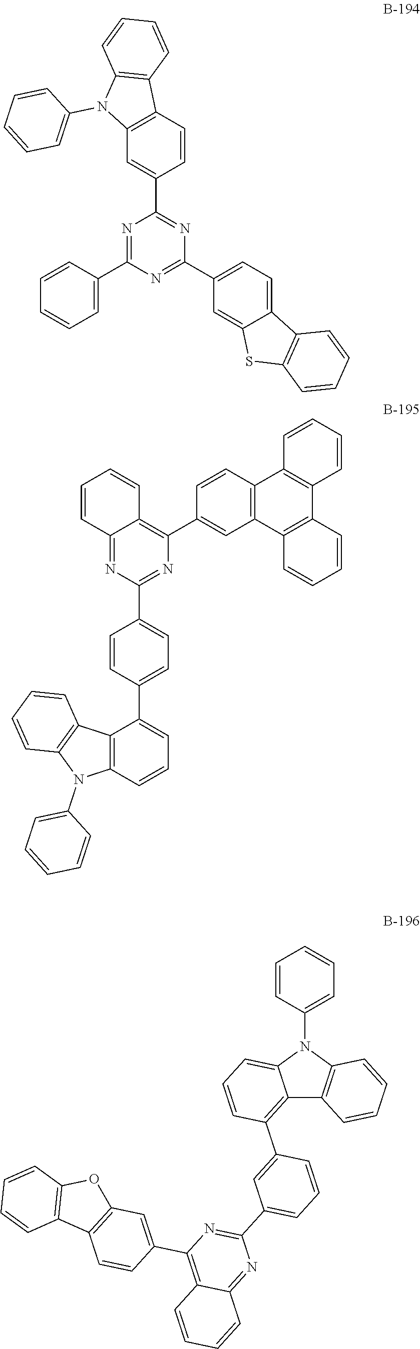

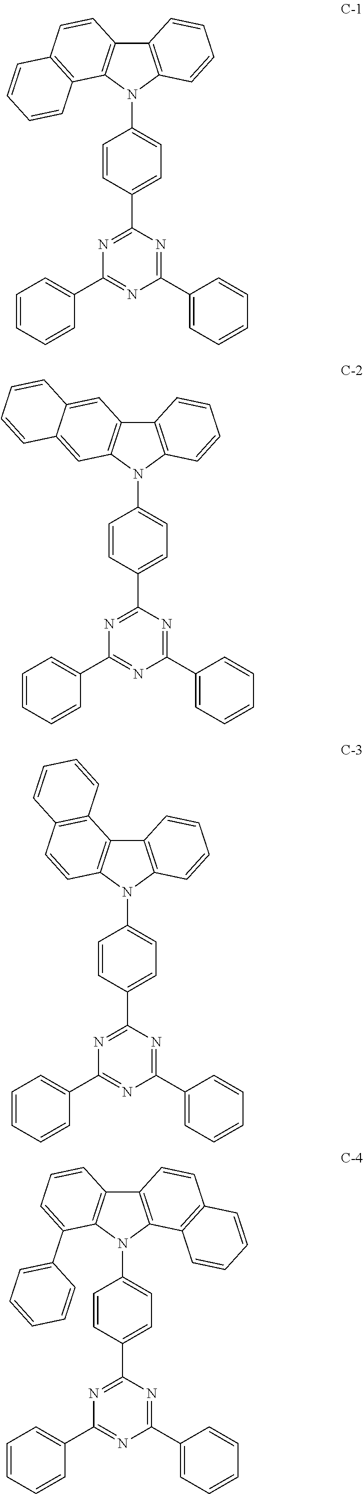

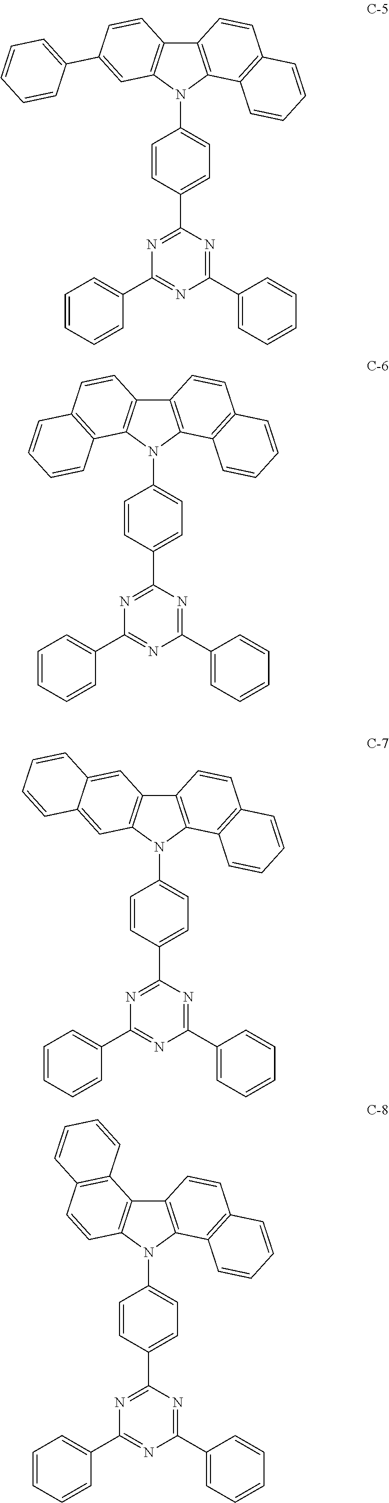

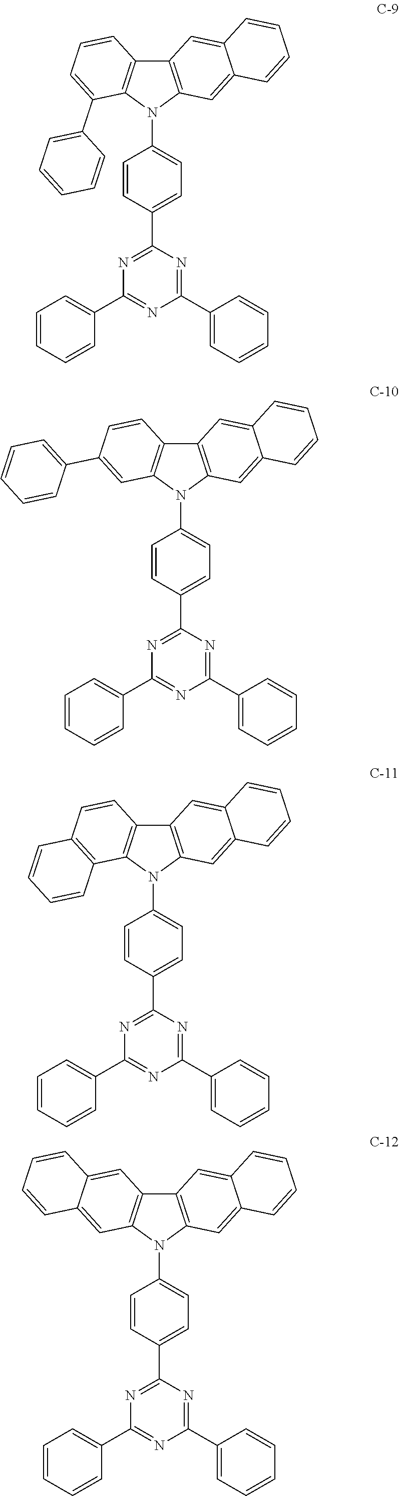

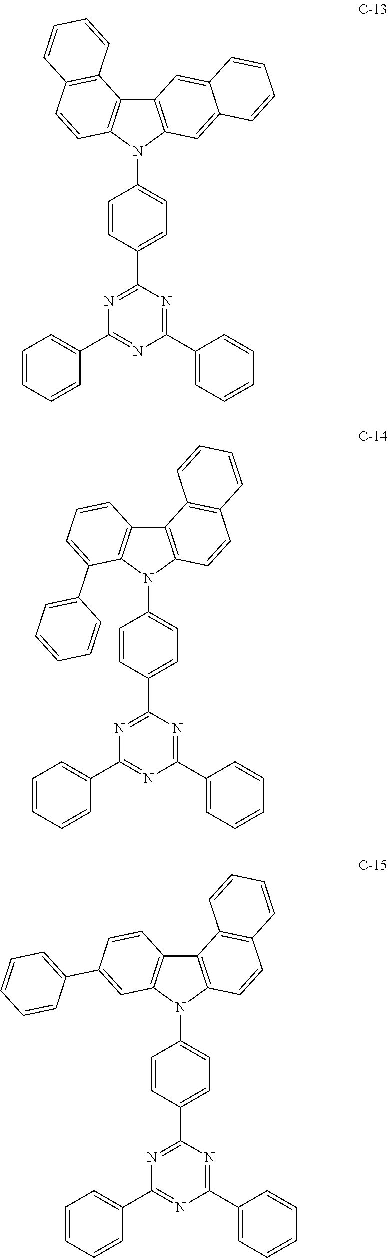

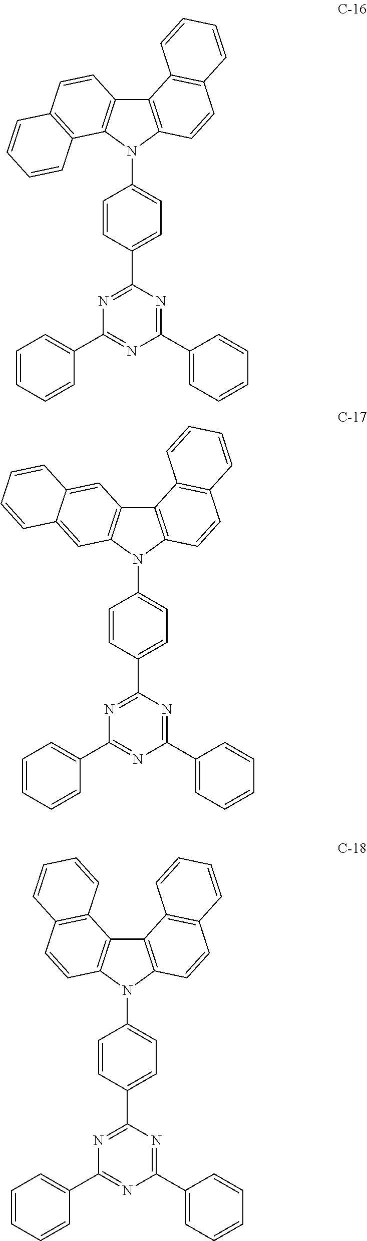

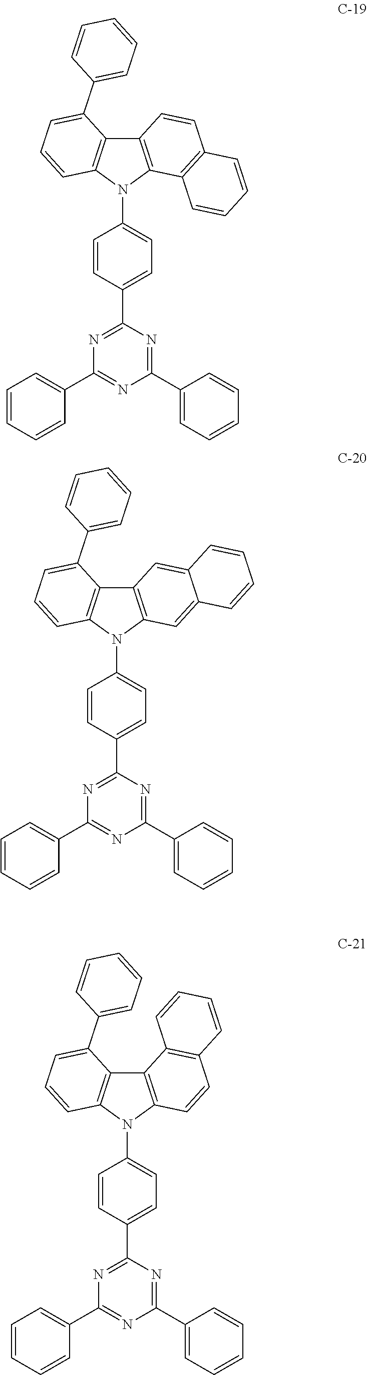 [SMILES: c1ccc(-c2nc(-c3ccccc3)nc(-c3ccc(-n4c5cccc(-c6ccccc6)c5c5ccc6ccccc6c54)cc3)n2)cc1 ; c1ccc(-c2nc(-c3ccccc3)nc(-c3ccc(-n4c5cc6ccccc6cc5c5c(-c6ccccc6)cccc54)cc3)n2)cc1 ; c1ccc(-c2nc(-c3ccccc3)nc(-c3ccc(-n4c5cccc(-c6ccccc6)c5c5c6ccccc6ccc54)cc3)n2)cc1]

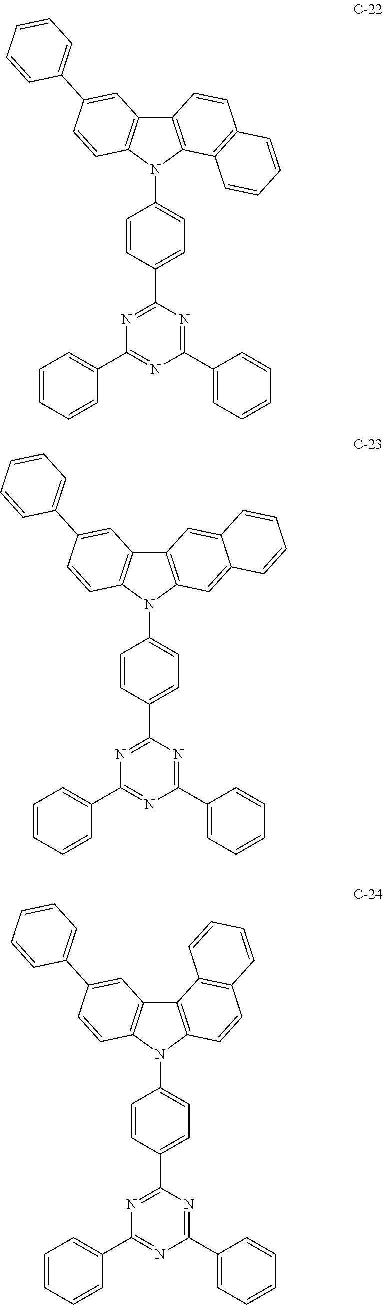

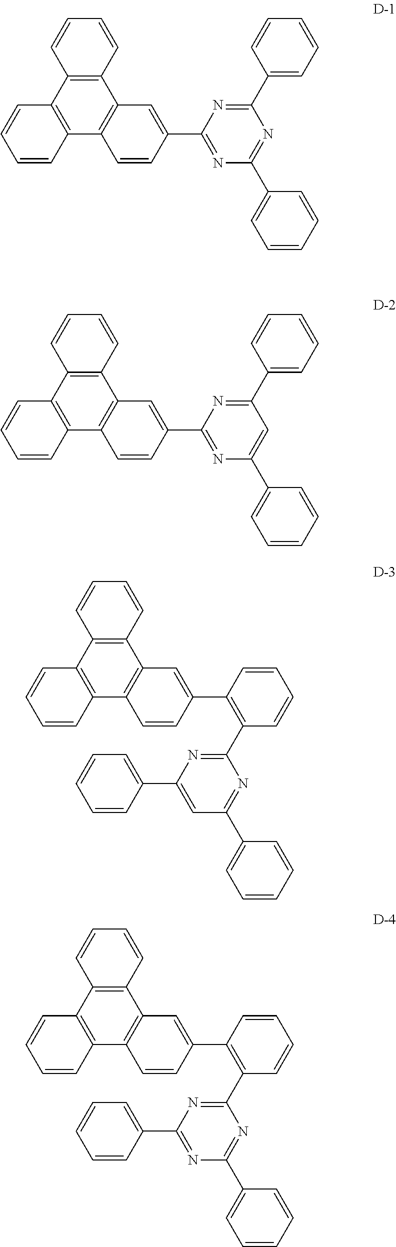 [SMILES: c1ccc(-c2nc(-c3ccccc3)nc(-c3ccc4c5ccccc5c5ccccc5c4c3)n2)cc1 ; c1ccc(-c2cc(-c3ccccc3)nc(-c3ccc4c5ccccc5c5ccccc5c4c3)n2)cc1 ; c1ccc(-c2cc(-c3ccccc3)nc(-c3ccccc3-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1]

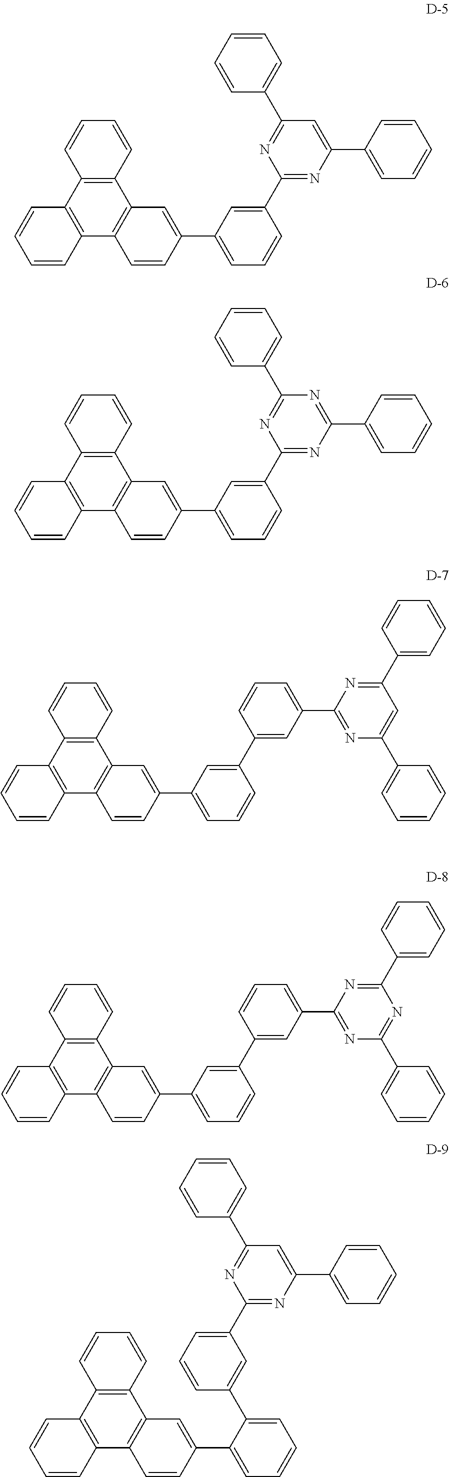

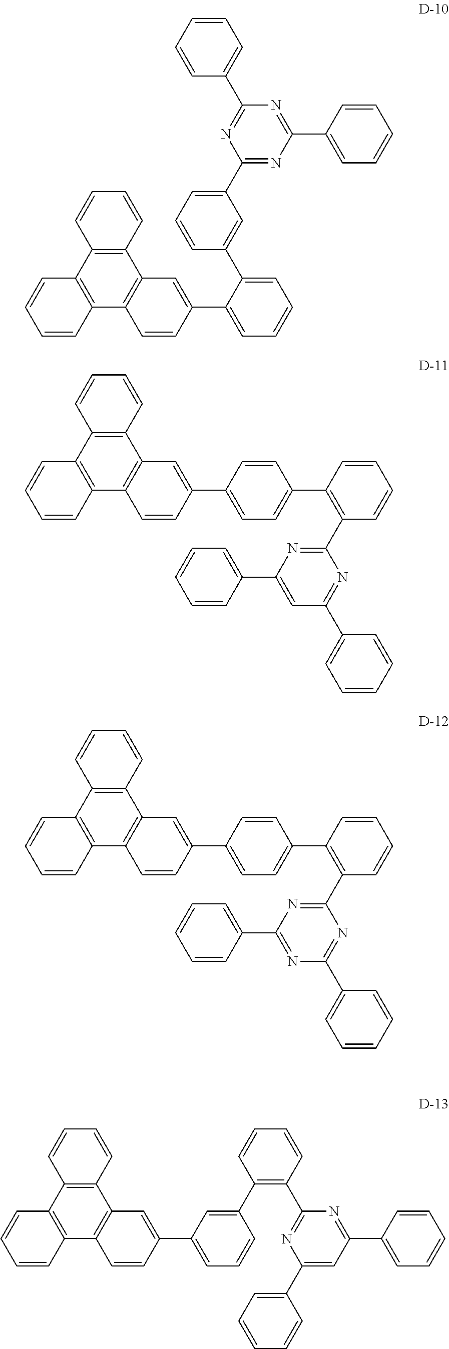

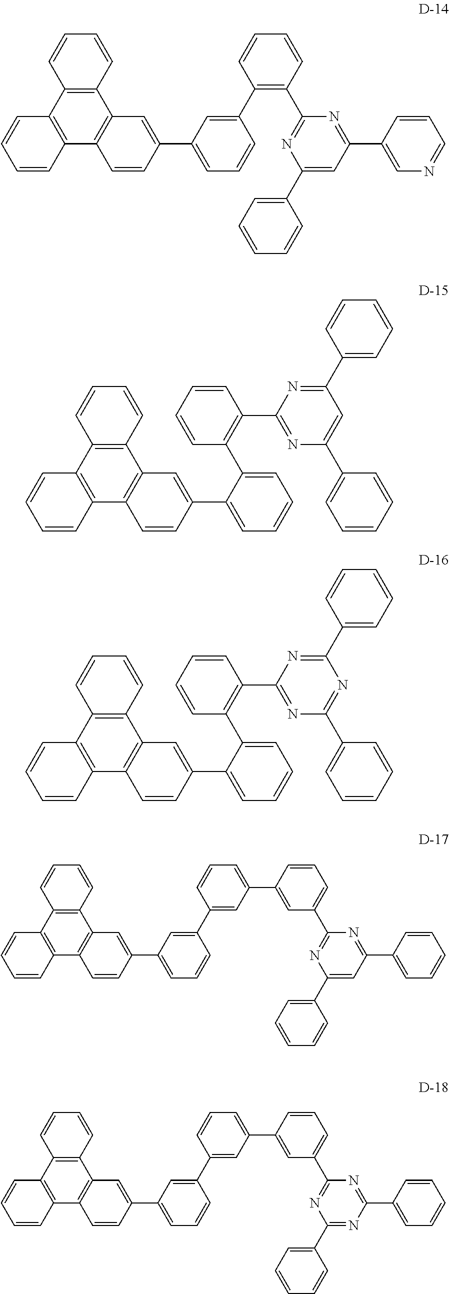

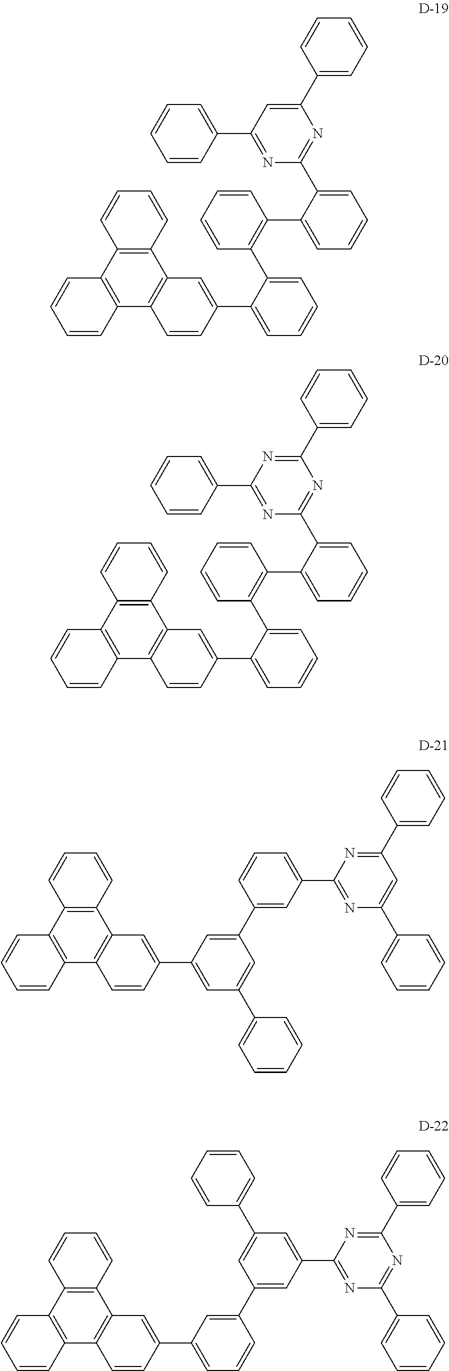

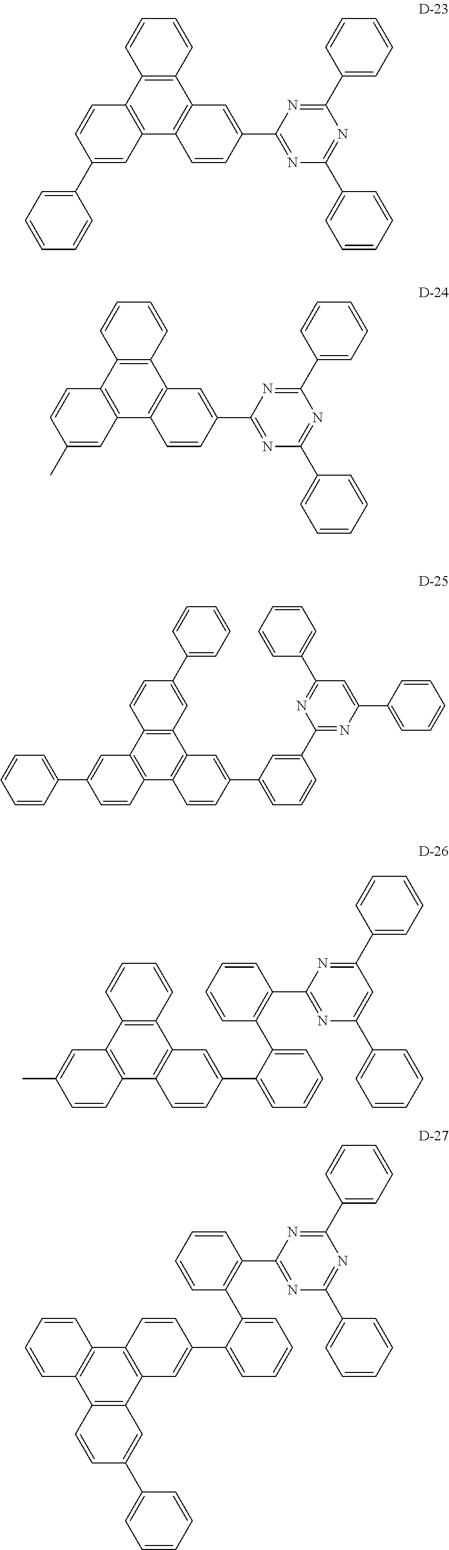

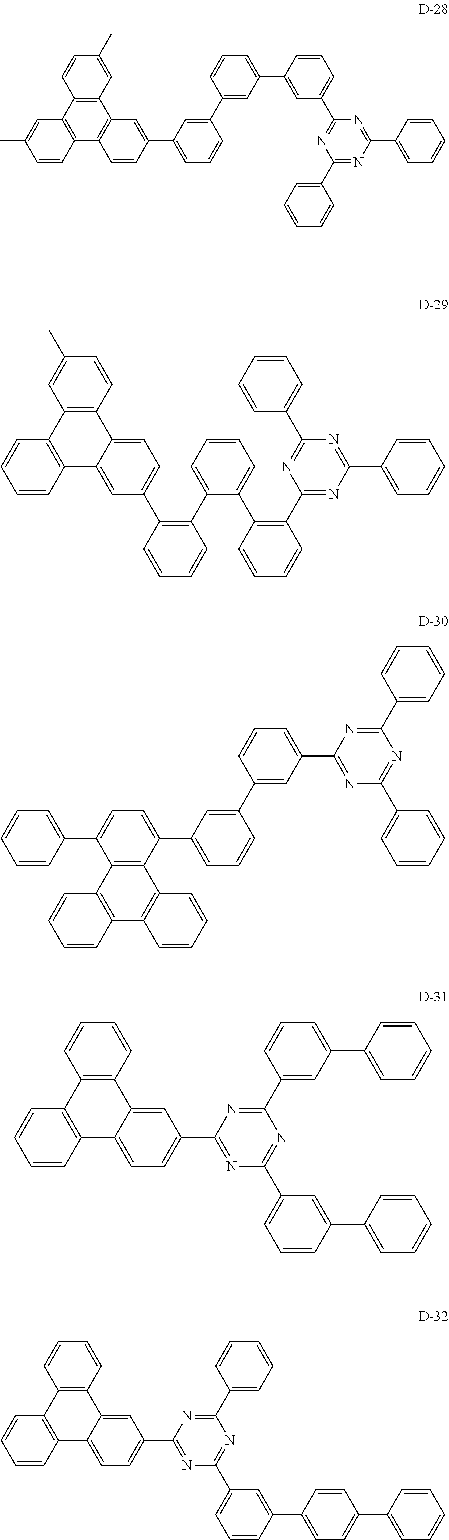 [SMILES: Cc1ccc2c3ccc(-c4cccc(-c5cccc(-c6cccc(-c7nc(-c8ccccc8)nc(-c8ccccc8)n7)c6)c5)c4)cc3c3cc(C)ccc3c2c1 ; Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2cccc(-c3nc(-c4cccc(-c5ccccc5)c4)nc(-c4ccc5c6ccccc6c6ccccc6c5c4)n3)c2)cc1 ; c1ccc(-c2ccc(-c3cccc(-c4nc(-c5ccccc5)nc(-c5ccc6c7ccccc7c7ccccc7c6c5)n4)c3)cc2)cc1]

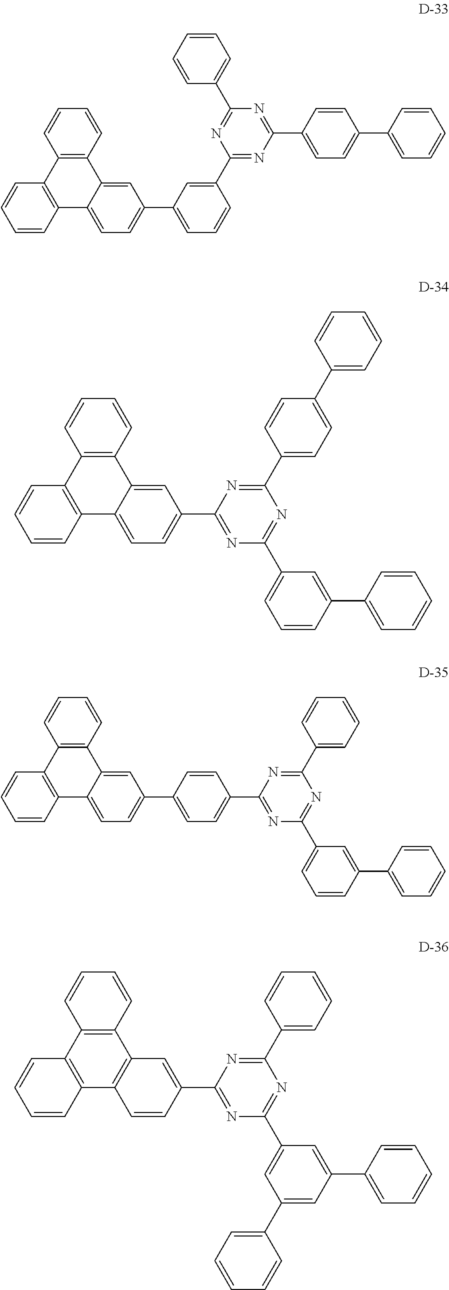 [SMILES: c1ccc(-c2ccc(-c3nc(-c4ccccc4)nc(-c4cccc(-c5ccc6c7ccccc7c7ccccc7c6c5)c4)n3)cc2)cc1 ; c1ccc(-c2ccc(-c3nc(-c4cccc(-c5ccccc5)c4)nc(-c4ccc5c6ccccc6c6ccccc6c5c4)n3)cc2)cc1 ; c1ccc(-c2cccc(-c3nc(-c4ccccc4)nc(-c4ccc(-c5ccc6c7ccccc7c7ccccc7c6c5)cc4)n3)c2)cc1 ; c1ccc(-c2cc(-c3ccccc3)cc(-c3nc(-c4ccccc4)nc(-c4ccc5c6ccccc6c6ccccc6c5c4)n3)c2)cc1]

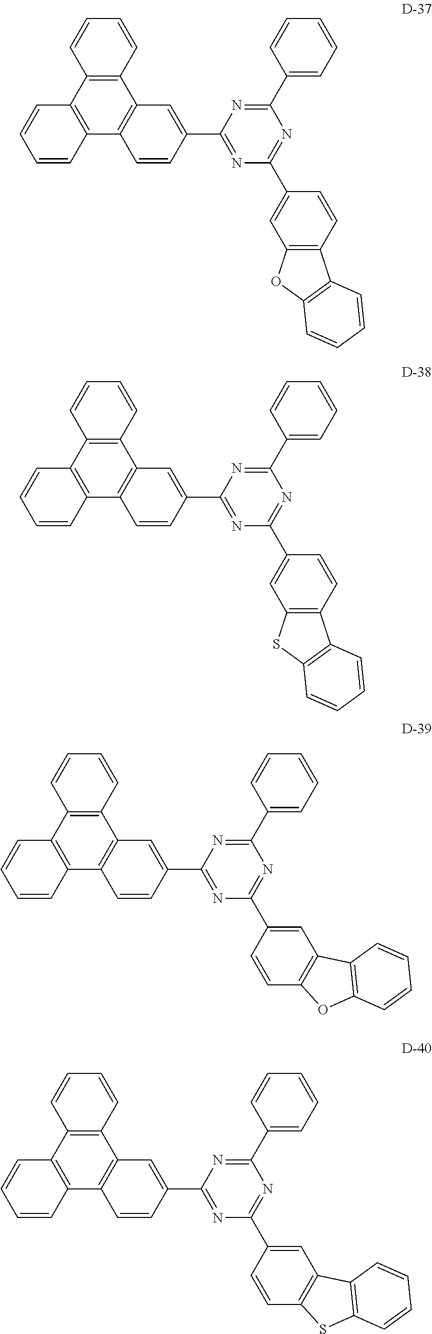

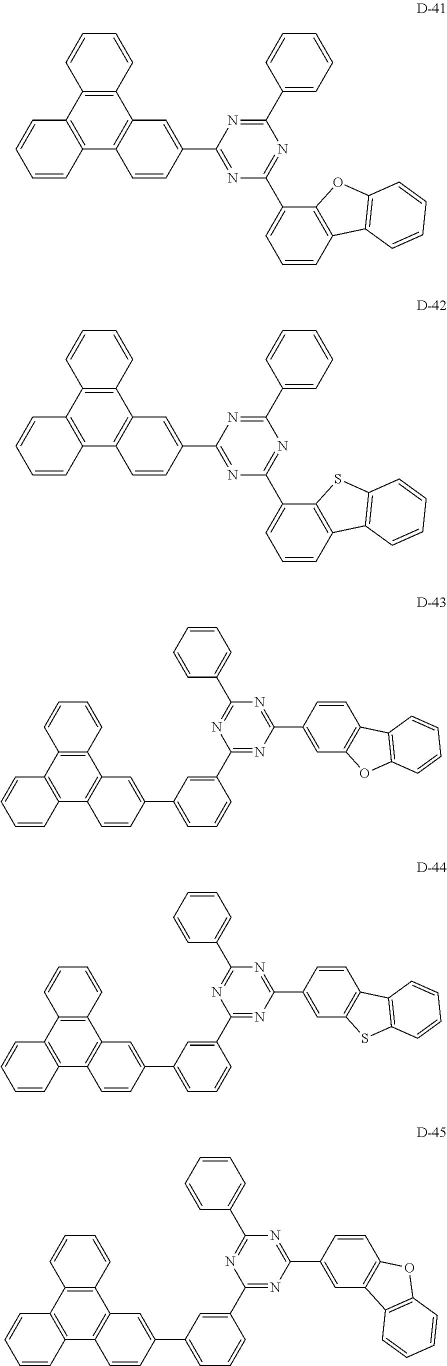 [SMILES: c1ccc(-c2nc(-c3ccc4c5ccccc5c5ccccc5c4c3)nc(-c3cccc4c3oc3ccccc34)n2)cc1 ; c1ccc(-c2nc(-c3ccc4c5ccccc5c5ccccc5c4c3)nc(-c3cccc4c3sc3ccccc34)n2)cc1 ; c1ccc(-c2nc(-c3cccc(-c4ccc5c6ccccc6c6ccccc6c5c4)c3)nc(-c3ccc4c(c3)oc3ccccc34)n2)cc1 ; c1ccc(-c2nc(-c3cccc(-c4ccc5c6ccccc6c6ccccc6c5c4)c3)nc(-c3ccc4c(c3)sc3ccccc34)n2)cc1 ; c1ccc(-c2nc(-c3cccc(-c4ccc5c6ccccc6c6ccccc6c5c4)c3)nc(-c3ccc4oc5ccccc5c4c3)n2)cc1]

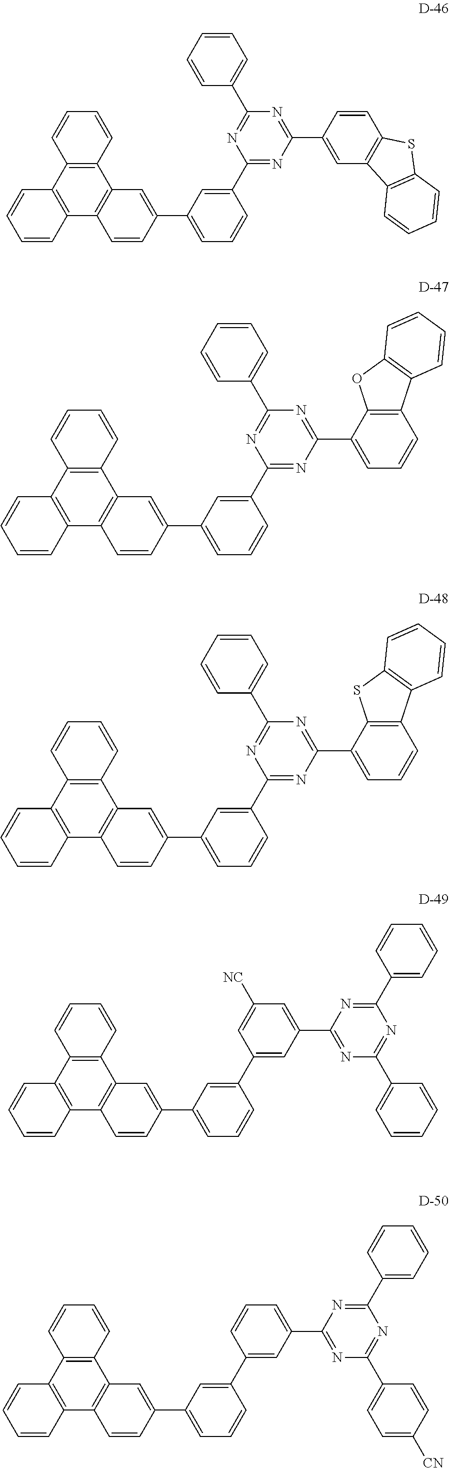 [SMILES: c1ccc(-c2nc(-c3cccc(-c4ccc5c6ccccc6c6ccccc6c5c4)c3)nc(-c3ccc4sc5ccccc5c4c3)n2)cc1 ; c1ccc(-c2nc(-c3cccc(-c4ccc5c6ccccc6c6ccccc6c5c4)c3)nc(-c3cccc4c3oc3ccccc34)n2)cc1 ; c1ccc(-c2nc(-c3cccc(-c4ccc5c6ccccc6c6ccccc6c5c4)c3)nc(-c3cccc4c3sc3ccccc34)n2)cc1 ; N#Cc1cc(-c2cccc(-c3ccc4c5ccccc5c5ccccc5c4c3)c2)cc(-c2nc(-c3ccccc3)nc(-c3ccccc3)n2)c1 ; N#Cc1ccc(-c2nc(-c3ccccc3)nc(-c3cccc(-c4cccc(-c5ccc6c7ccccc7c7ccccc7c6c5)c4)c3)n2)cc1]

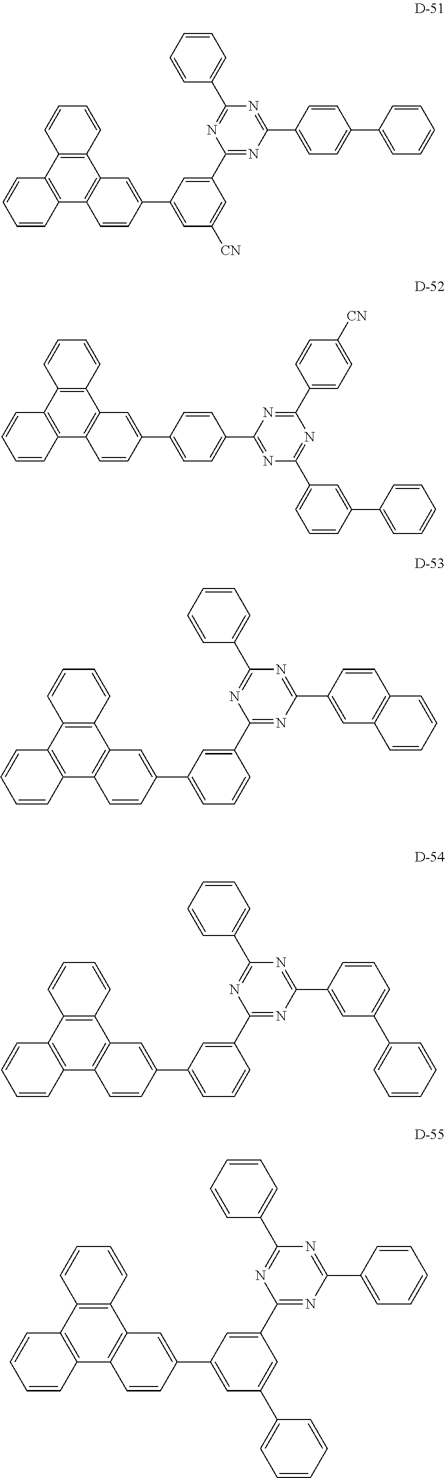 [SMILES: N#Cc1cc(-c2ccc3c4ccccc4c4ccccc4c3c2)cc(-c2nc(-c3ccccc3)nc(-c3ccc(-c4ccccc4)cc3)n2)c1 ; N#Cc1ccc(-c2nc(-c3ccc(-c4ccc5c6ccccc6c6ccccc6c5c4)cc3)nc(-c3cccc(-c4ccccc4)c3)n2)cc1 ; c1ccc(-c2nc(-c3cccc(-c4ccc5c6ccccc6c6ccccc6c5c4)c3)nc(-c3ccc4ccccc4c3)n2)cc1 ; c1ccc(-c2cccc(-c3nc(-c4ccccc4)nc(-c4cccc(-c5ccc6c7ccccc7c7ccccc7c6c5)c4)n3)c2)cc1 ; c1ccc(-c2cc(-c3ccc4c5ccccc5c5ccccc5c4c3)cc(-c3nc(-c4ccccc4)nc(-c4ccccc4)n3)c2)cc1]

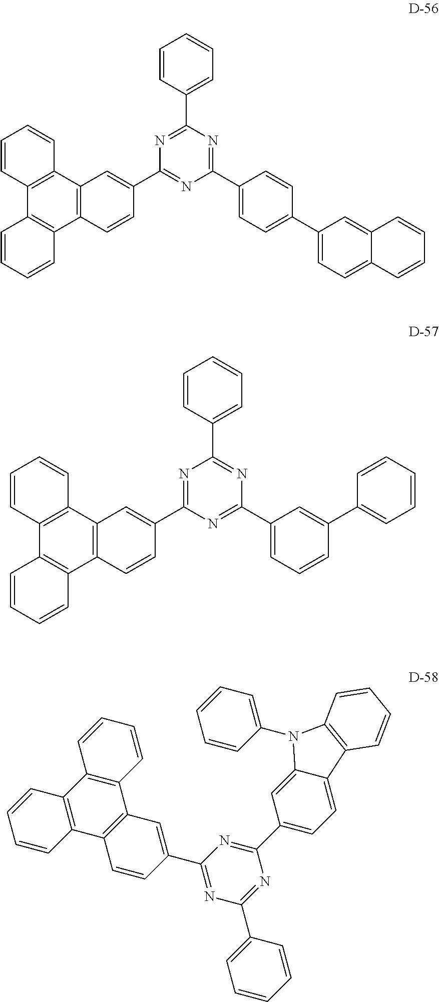 [SMILES: c1ccc(-c2nc(-c3ccc(-c4ccc5ccccc5c4)cc3)nc(-c3ccc4c5ccccc5c5ccccc5c4c3)n2)cc1 ; c1ccc(-c2cccc(-c3nc(-c4ccccc4)nc(-c4ccc5c6ccccc6c6ccccc6c5c4)n3)c2)cc1 ; c1ccc(-c2nc(-c3ccc4c5ccccc5c5ccccc5c4c3)nc(-c3ccc4c5ccccc5n(-c5ccccc5)c4c3)n2)cc1]

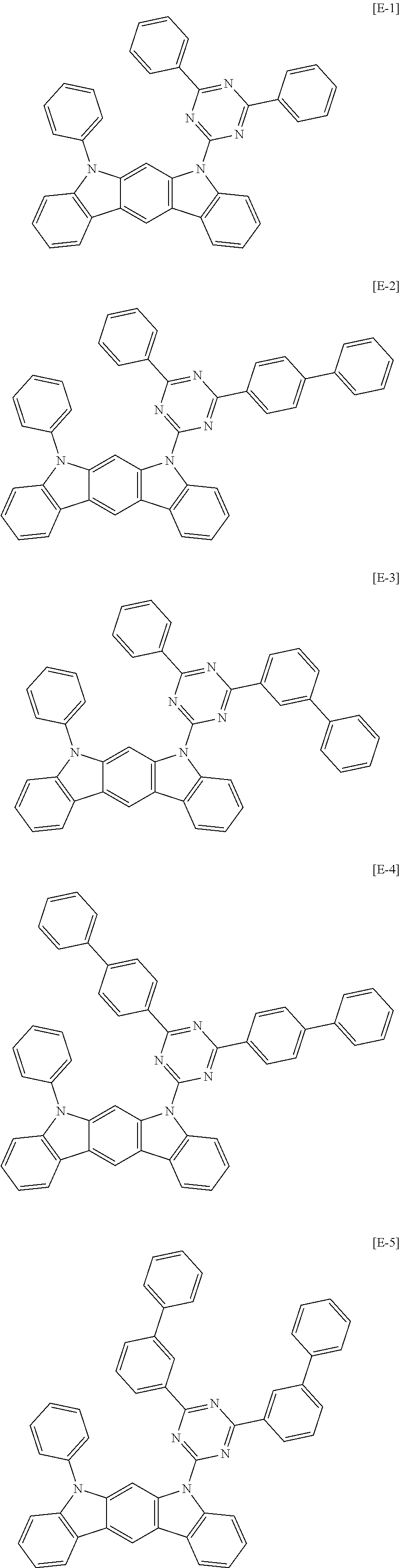 [SMILES: c1ccc(-c2nc(-c3ccccc3)nc(-n3c4ccccc4c4cc5c6ccccc6n(-c6ccccc6)c5cc43)n2)cc1 ; c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2ccc(-c3nc(-c4ccc(-c5ccccc5)cc4)nc(-n4c5ccccc5c5cc6c7ccccc7n(-c7ccccc7)c6cc54)n3)cc2)cc1 ; c1ccc(-c2cccc(-c3nc(-c4cccc(-c5ccccc5)c4)nc(-n4c5ccccc5c5cc6c7ccccc7n(-c7ccccc7)c6cc54)n3)c2)cc1]

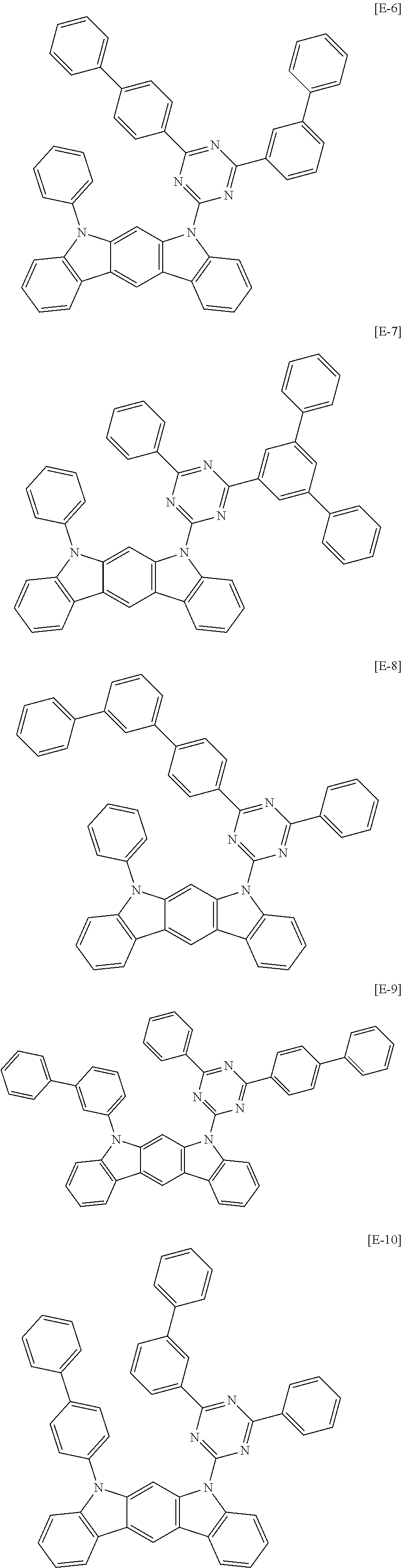 [SMILES: c1ccc(-c2ccc(-c3nc(-c4cccc(-c5ccccc5)c4)nc(-n4c5ccccc5c5cc6c7ccccc7n(-c7ccccc7)c6cc54)n3)cc2)cc1 ; c1ccc(-c2cc(-c3ccccc3)cc(-c3nc(-c4ccccc4)nc(-n4c5ccccc5c5cc6c7ccccc7n(-c7ccccc7)c6cc54)n3)c2)cc1 ; c1ccc(-c2cccc(-c3ccc(-c4nc(-c5ccccc5)nc(-n5c6ccccc6c6cc7c8ccccc8n(-c8ccccc8)c7cc65)n4)cc3)c2)cc1 ; c1ccc(-c2ccc(-c3nc(-c4ccccc4)nc(-n4c5ccccc5c5cc6c7ccccc7n(-c7cccc(-c8ccccc8)c7)c6cc54)n3)cc2)cc1 ; c1ccc(-c2ccc(-n3c4ccccc4c4cc5c6ccccc6n(-c6nc(-c7ccccc7)nc(-c7cccc(-c8ccccc8)c7)n6)c5cc43)cc2)cc1]

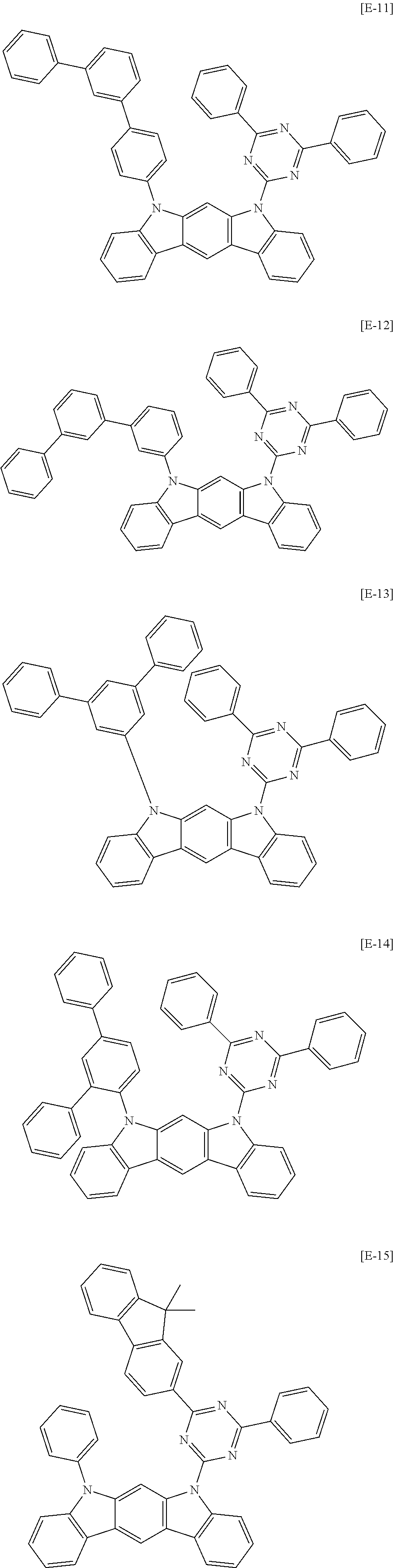 [SMILES: c1ccc(-c2cccc(-c3ccc(-n4c5ccccc5c5cc6c7ccccc7n(-c7nc(-c8ccccc8)nc(-c8ccccc8)n7)c6cc54)cc3)c2)cc1 ; c1ccc(-c2cccc(-c3cccc(-n4c5ccccc5c5cc6c7ccccc7n(-c7nc(-c8ccccc8)nc(-c8ccccc8)n7)c6cc54)c3)c2)cc1 ; c1ccc(-c2cc(-c3ccccc3)cc(-n3c4ccccc4c4cc5c6ccccc6n(-c6nc(-c7ccccc7)nc(-c7ccccc7)n6)c5cc43)c2)cc1 ; c1ccc(-c2ccc(-n3c4ccccc4c4cc5c6ccccc6n(-c6nc(-c7ccccc7)nc(-c7ccccc7)n6)c5cc43)c(-c3ccccc3)c2)cc1 ; CC1(C)c2ccccc2-c2ccc(-c3nc(-c4ccccc4)nc(-n4c5ccccc5c5cc6c7ccccc7n(-c7ccccc7)c6cc54)n3)cc21]

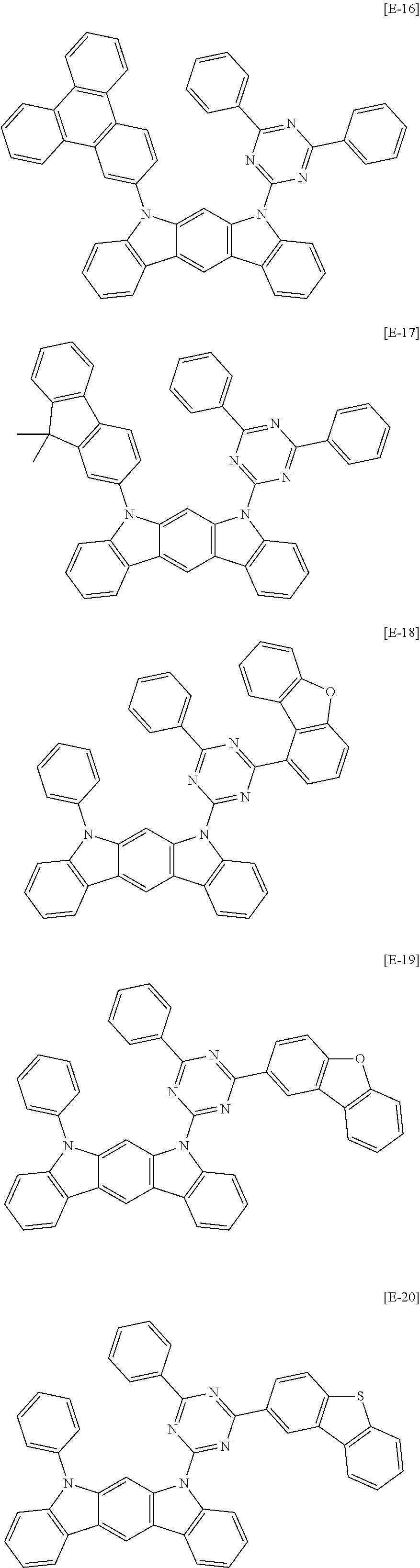 [SMILES: c1ccc(-c2nc(-c3ccccc3)nc(-n3c4ccccc4c4cc5c6ccccc6n(-c6ccc7c8ccccc8c8ccccc8c7c6)c5cc43)n2)cc1 ; CC1(C)c2ccccc2-c2ccc(-n3c4ccccc4c4cc5c6ccccc6n(-c6nc(-c7ccccc7)nc(-c7ccccc7)n6)c5cc43)cc21 ; c1ccc(-c2nc(-c3cccc4oc5ccccc5c34)nc(-n3c4ccccc4c4cc5c6ccccc6n(-c6ccccc6)c5cc43)n2)cc1 ; c1ccc(-c2nc(-c3ccc4oc5ccccc5c4c3)nc(-n3c4ccccc4c4cc5c6ccccc6n(-c6ccccc6)c5cc43)n2)cc1 ; c1ccc(-c2nc(-c3ccc4sc5ccccc5c4c3)nc(-n3c4ccccc4c4cc5c6ccccc6n(-c6ccccc6)c5cc43)n2)cc1]

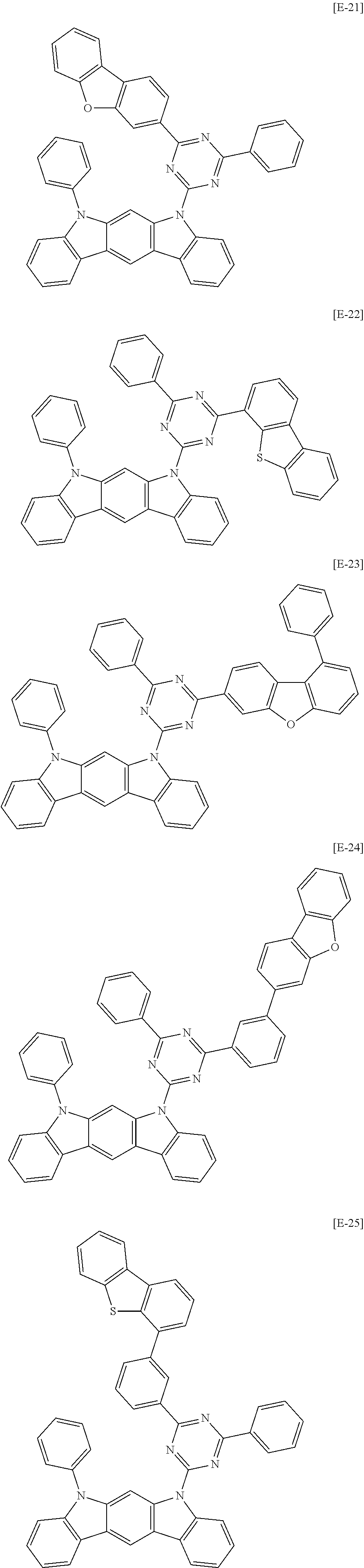 [SMILES: c1ccc(-c2nc(-c3ccc4c(c3)oc3ccccc34)nc(-n3c4ccccc4c4cc5c6ccccc6n(-c6ccccc6)c5cc43)n2)cc1 ; c1ccc(-c2nc(-c3cccc4c3sc3ccccc34)nc(-n3c4ccccc4c4cc5c6ccccc6n(-c6ccccc6)c5cc43)n2)cc1 ; c1ccc(-c2nc(-c3ccc4c(c3)oc3cccc(-c5ccccc5)c34)nc(-n3c4ccccc4c4cc5c6ccccc6n(-c6ccccc6)c5cc43)n2)cc1 ; c1ccc(-c2nc(-c3cccc(-c4ccc5c(c4)oc4ccccc45)c3)nc(-n3c4ccccc4c4cc5c6ccccc6n(-c6ccccc6)c5cc43)n2)cc1 ; c1ccc(-c2nc(-c3cccc(-c4cccc5c4sc4ccccc45)c3)nc(-n3c4ccccc4c4cc5c6ccccc6n(-c6ccccc6)c5cc43)n2)cc1]

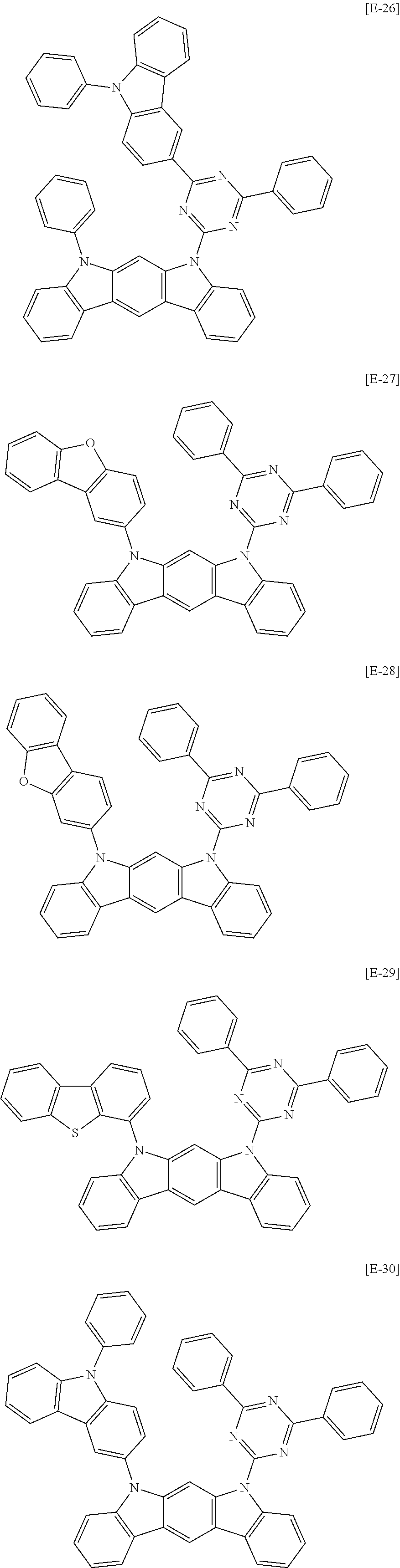 [SMILES: c1ccc(-c2nc(-c3ccc4c(c3)c3ccccc3n4-c3ccccc3)nc(-n3c4ccccc4c4cc5c6ccccc6n(-c6ccccc6)c5cc43)n2)cc1 ; c1ccc(-c2nc(-c3ccccc3)nc(-n3c4ccccc4c4cc5c6ccccc6n(-c6ccc7oc8ccccc8c7c6)c5cc43)n2)cc1 ; c1ccc(-c2nc(-c3ccccc3)nc(-n3c4ccccc4c4cc5c6ccccc6n(-c6ccc7c(c6)oc6ccccc67)c5cc43)n2)cc1 ; c1ccc(-c2nc(-c3ccccc3)nc(-n3c4ccccc4c4cc5c6ccccc6n(-c6cccc7c6sc6ccccc67)c5cc43)n2)cc1 ; c1ccc(-c2nc(-c3ccccc3)nc(-n3c4ccccc4c4cc5c6ccccc6n(-c6ccc7c(c6)c6ccccc6n7-c6ccccc6)c5cc43)n2)cc1]

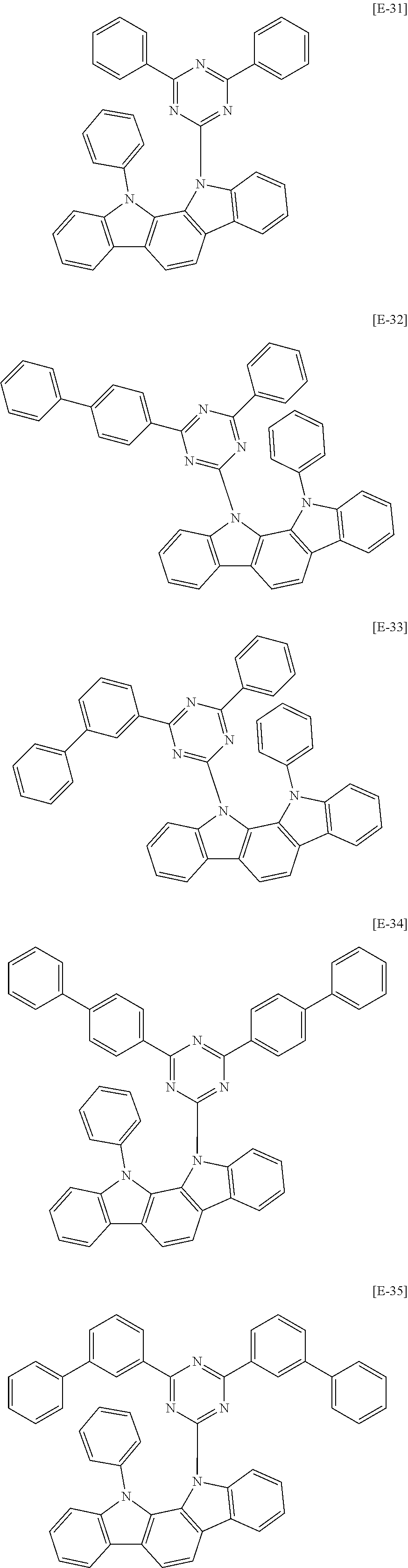 [SMILES: c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2cccc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1 ; c1ccc(-c2cccc(-c3nc(-c4cccc(-c5ccccc5)c4)nc(-n4c5ccccc5c5ccc6c7ccccc7n(-c7ccccc7)c6c54)n3)c2)cc1]

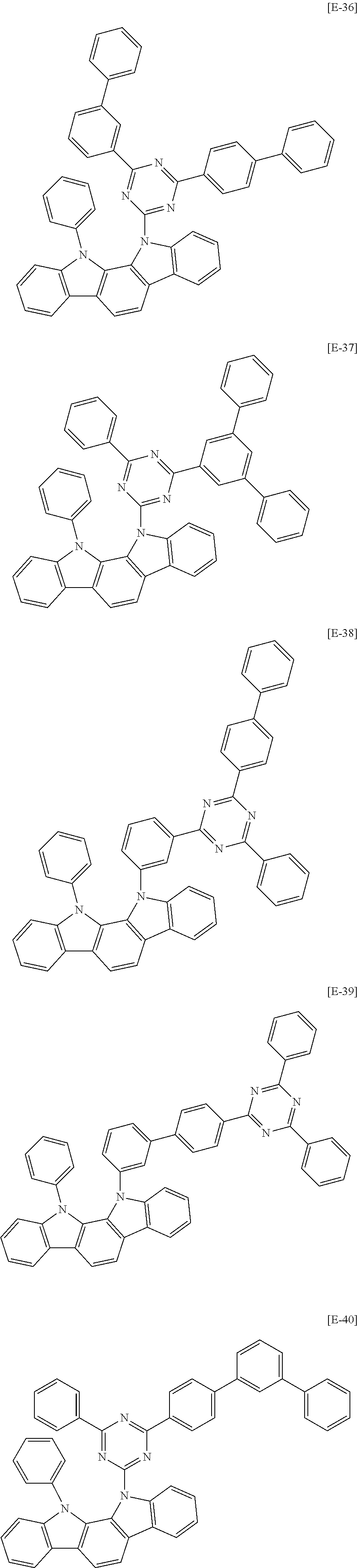 [SMILES: c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1 ; c1ccc(-c2cc(-c3ccccc3)cc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccccc4)nc(-c4cccc(-n5c6ccccc6c6ccc7c8ccccc8n(-c8ccccc8)c7c65)c4)n3)cc2)cc1 ; c1ccc(-c2nc(-c3ccccc3)nc(-c3ccc(-c4cccc(-n5c6ccccc6c6ccc7c8ccccc8n(-c8ccccc8)c7c65)c4)cc3)n2)cc1 ; c1ccc(-c2cccc(-c3ccc(-c4nc(-c5ccccc5)nc(-n5c6ccccc6c6ccc7c8ccccc8n(-c8ccccc8)c7c65)n4)cc3)c2)cc1]

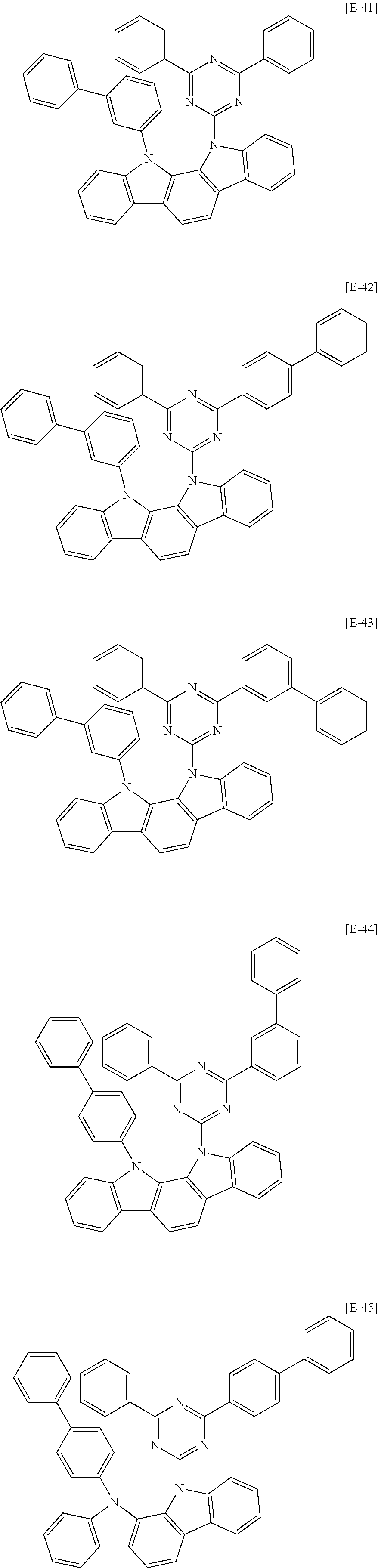 [SMILES: c1ccc(-c2cccc(-n3c4ccccc4c4ccc5c6ccccc6n(-c6nc(-c7ccccc7)nc(-c7ccccc7)n6)c5c43)c2)cc1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2cccc(-c3nc(-c4ccccc4)nc(-n4c5ccccc5c5ccc6c7ccccc7n(-c7cccc(-c8ccccc8)c7)c6c54)n3)c2)cc1 ; c1ccc(-c2ccc(-n3c4ccccc4c4ccc5c6ccccc6n(-c6nc(-c7ccccc7)nc(-c7cccc(-c8ccccc8)c7)n6)c5c43)cc2)cc1 ; c1ccc(-c2ccc(-c3nc(-c4ccccc4)nc(-n4c5ccccc5c5ccc6c7ccccc7n(-c7ccc(-c8ccccc8)cc7)c6c54)n3)cc2)cc1]

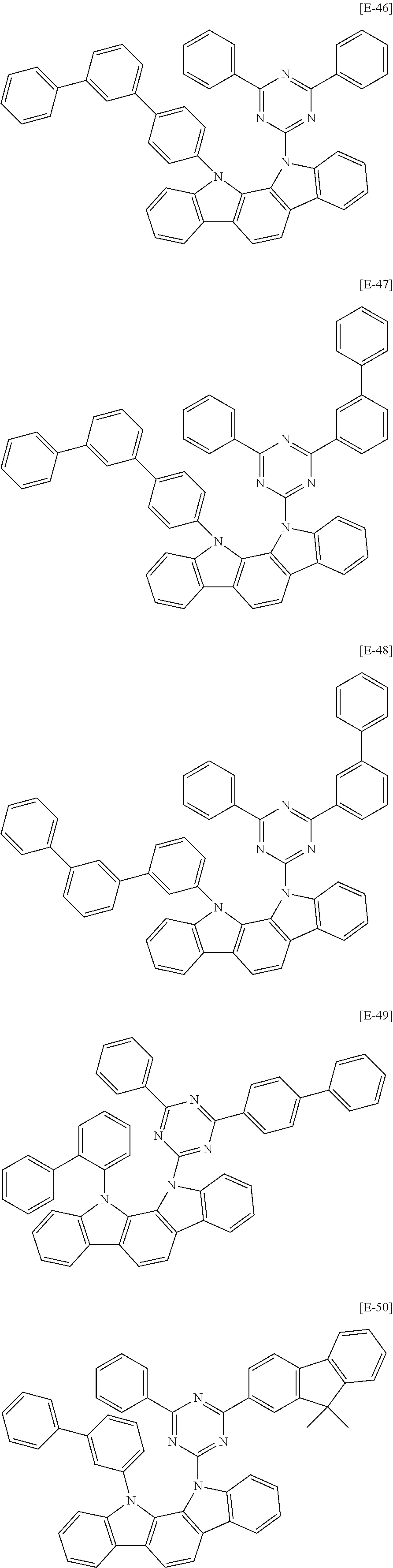

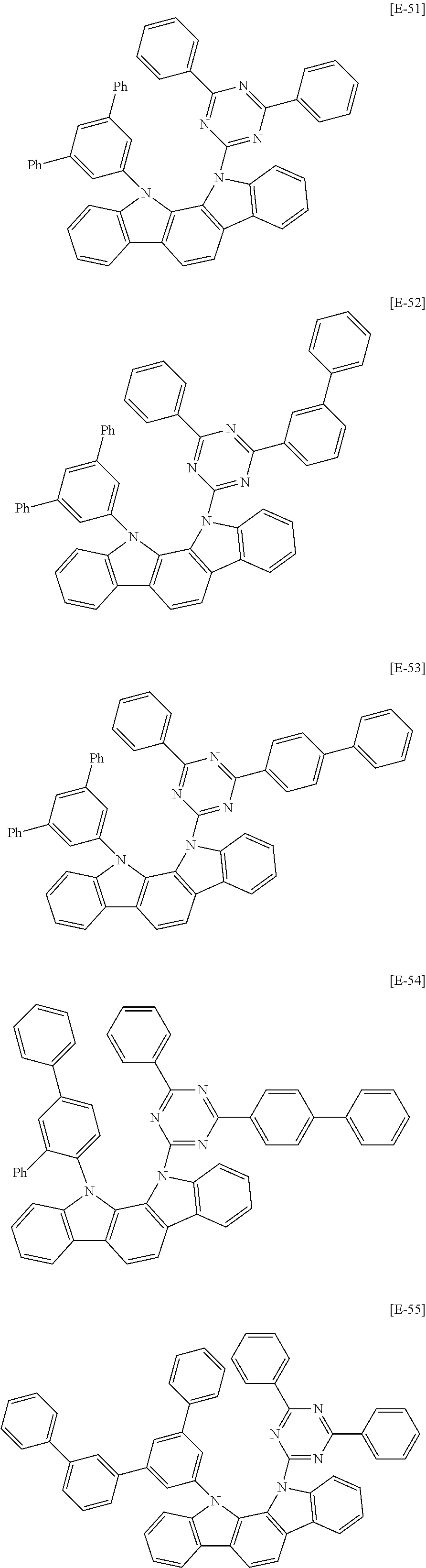

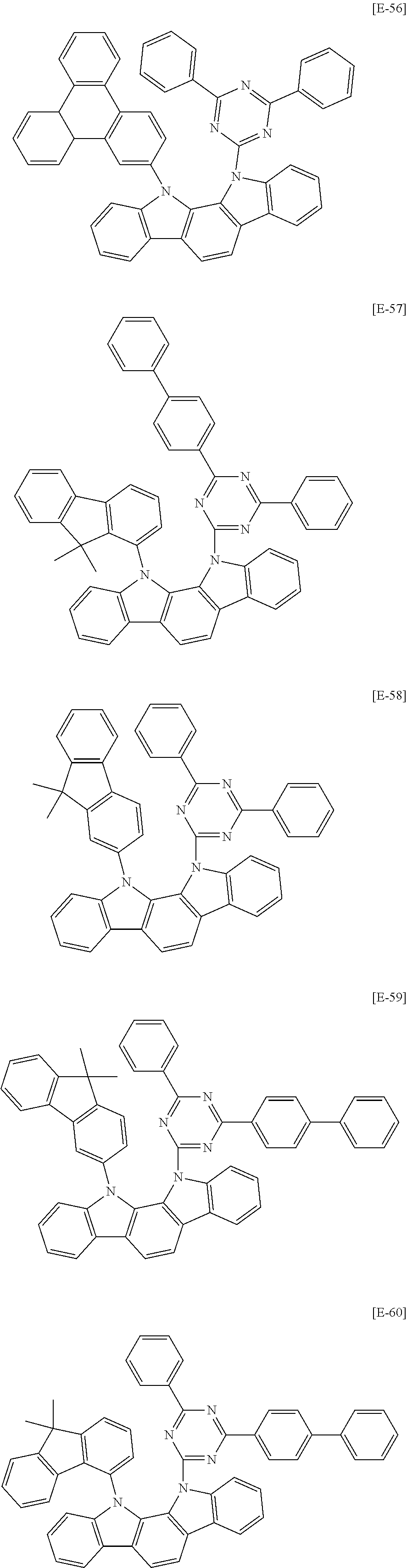 [SMILES: C1=CC2c3ccccc3-c3ccc(-n4c5ccccc5c5ccc6c7ccccc7n(-c7nc(-c8ccccc8)nc(-c8ccccc8)n7)c6c54)cc3C2C=C1 ; CC1(C)c2ccccc2-c2cccc(-n3c4ccccc4c4ccc5c6ccccc6n(-c6nc(-c7ccccc7)nc(-c7ccc(-c8ccccc8)cc7)n6)c5c43)c21 ; CC1(C)c2ccccc2-c2ccc(-n3c4ccccc4c4ccc5c6ccccc6n(-c6nc(-c7ccccc7)nc(-c7ccccc7)n6)c5c43)cc21 ; CC1(C)c2ccccc2-c2cc(-n3c4ccccc4c4ccc5c6ccccc6n(-c6nc(-c7ccccc7)nc(-c7ccc(-c8ccccc8)cc7)n6)c5c43)ccc21 ; CC1(C)c2ccccc2-c2c(-n3c4ccccc4c4ccc5c6ccccc6n(-c6nc(-c7ccccc7)nc(-c7ccc(-c8ccccc8)cc7)n6)c5c43)cccc21]

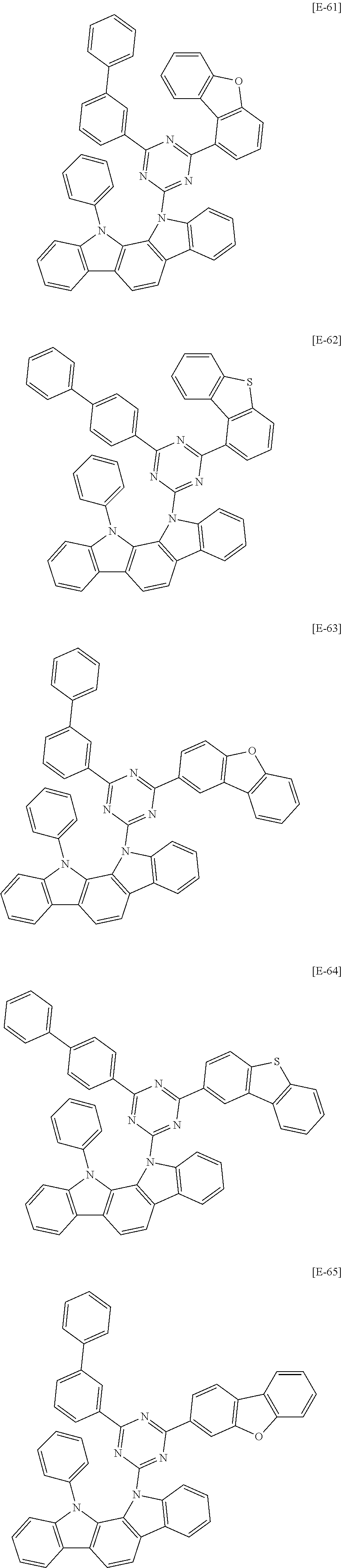 [SMILES: c1ccc(-c2cccc(-c3nc(-c4cccc5oc6ccccc6c45)nc(-n4c5ccccc5c5ccc6c7ccccc7n(-c7ccccc7)c6c54)n3)c2)cc1 ; c1ccc(-c2ccc(-c3nc(-c4cccc5sc6ccccc6c45)nc(-n4c5ccccc5c5ccc6c7ccccc7n(-c7ccccc7)c6c54)n3)cc2)cc1 ; c1ccc(-c2cccc(-c3nc(-c4ccc5oc6ccccc6c5c4)nc(-n4c5ccccc5c5ccc6c7ccccc7n(-c7ccccc7)c6c54)n3)c2)cc1 ; c1ccc(-c2ccc(-c3nc(-c4ccc5sc6ccccc6c5c4)nc(-n4c5ccccc5c5ccc6c7ccccc7n(-c7ccccc7)c6c54)n3)cc2)cc1 ; c1ccc(-c2cccc(-c3nc(-c4ccc5c(c4)oc4ccccc45)nc(-n4c5ccccc5c5ccc6c7ccccc7n(-c7ccccc7)c6c54)n3)c2)cc1]

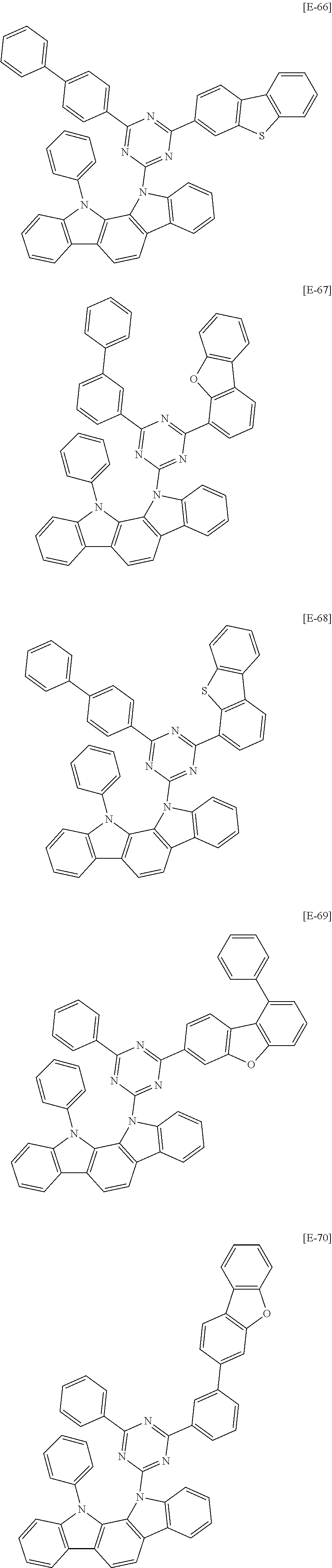

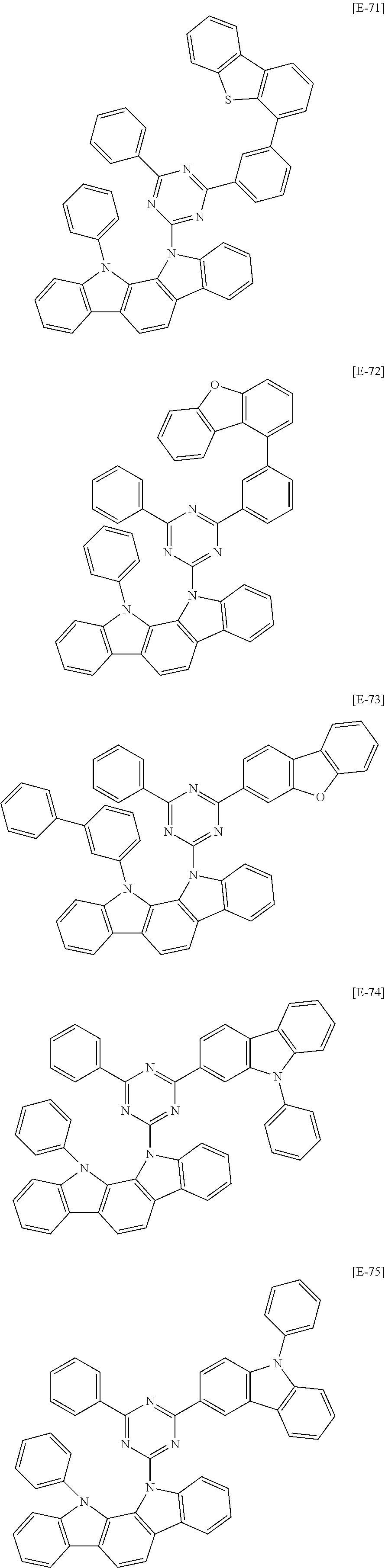 [SMILES: c1ccc(-c2nc(-c3cccc(-c4cccc5c4sc4ccccc45)c3)nc(-n3c4ccccc4c4ccc5c6ccccc6n(-c6ccccc6)c5c43)n2)cc1 ; c1ccc(-c2nc(-c3cccc(-c4cccc5oc6ccccc6c45)c3)nc(-n3c4ccccc4c4ccc5c6ccccc6n(-c6ccccc6)c5c43)n2)cc1 ; c1ccc(-c2cccc(-n3c4ccccc4c4ccc5c6ccccc6n(-c6nc(-c7ccccc7)nc(-c7ccc8c(c7)oc7ccccc78)n6)c5c43)c2)cc1 ; c1ccc(-c2nc(-c3ccc4c5ccccc5n(-c5ccccc5)c4c3)nc(-n3c4ccccc4c4ccc5c6ccccc6n(-c6ccccc6)c5c43)n2)cc1 ; c1ccc(-c2nc(-c3ccc4c(c3)c3ccccc3n4-c3ccccc3)nc(-n3c4ccccc4c4ccc5c6ccccc6n(-c6ccccc6)c5c43)n2)cc1]

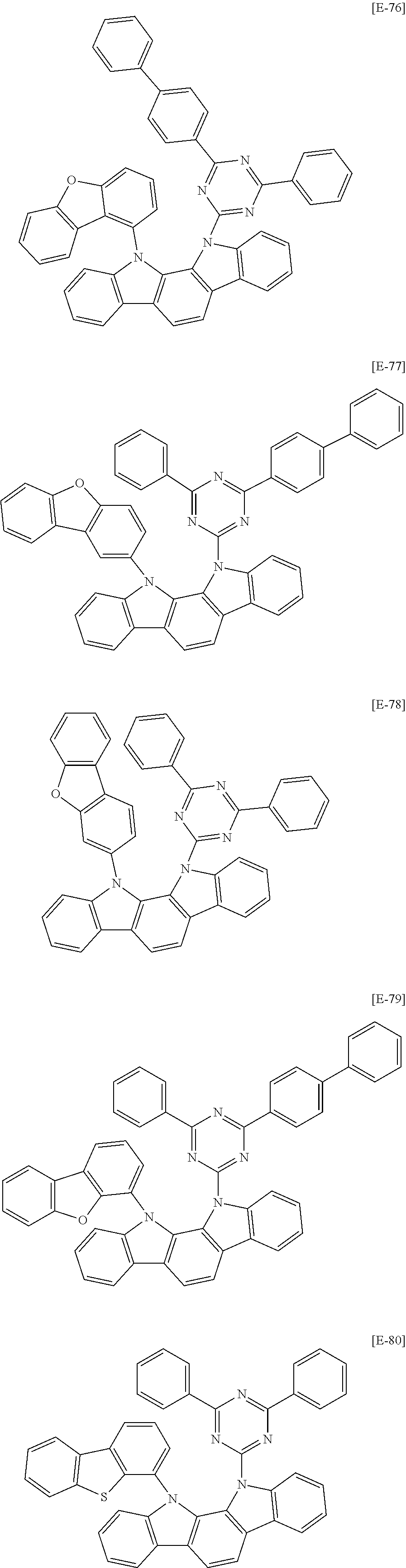 [SMILES: c1ccc(-c2ccc(-c3nc(-c4ccccc4)nc(-n4c5ccccc5c5ccc6c7ccccc7n(-c7cccc8oc9ccccc9c78)c6c54)n3)cc2)cc1 ; c1ccc(-c2ccc(-c3nc(-c4ccccc4)nc(-n4c5ccccc5c5ccc6c7ccccc7n(-c7ccc8oc9ccccc9c8c7)c6c54)n3)cc2)cc1 ; c1ccc(-c2nc(-c3ccccc3)nc(-n3c4ccccc4c4ccc5c6ccccc6n(-c6ccc7c(c6)oc6ccccc67)c5c43)n2)cc1 ; c1ccc(-c2ccc(-c3nc(-c4ccccc4)nc(-n4c5ccccc5c5ccc6c7ccccc7n(-c7cccc8c7oc7ccccc78)c6c54)n3)cc2)cc1 ; c1ccc(-c2nc(-c3ccccc3)nc(-n3c4ccccc4c4ccc5c6ccccc6n(-c6cccc7c6sc6ccccc67)c5c43)n2)cc1]

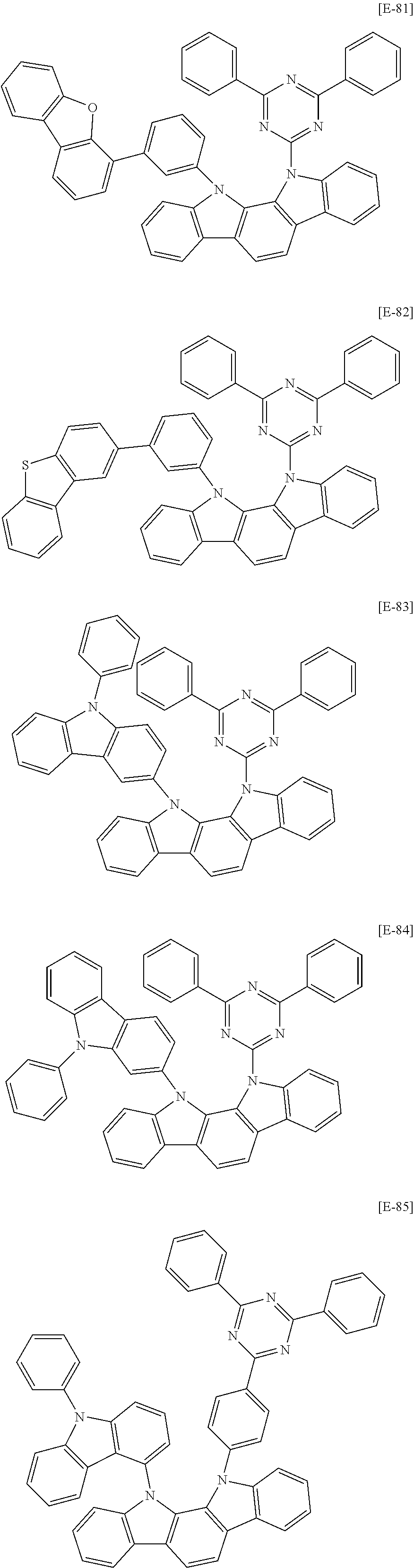

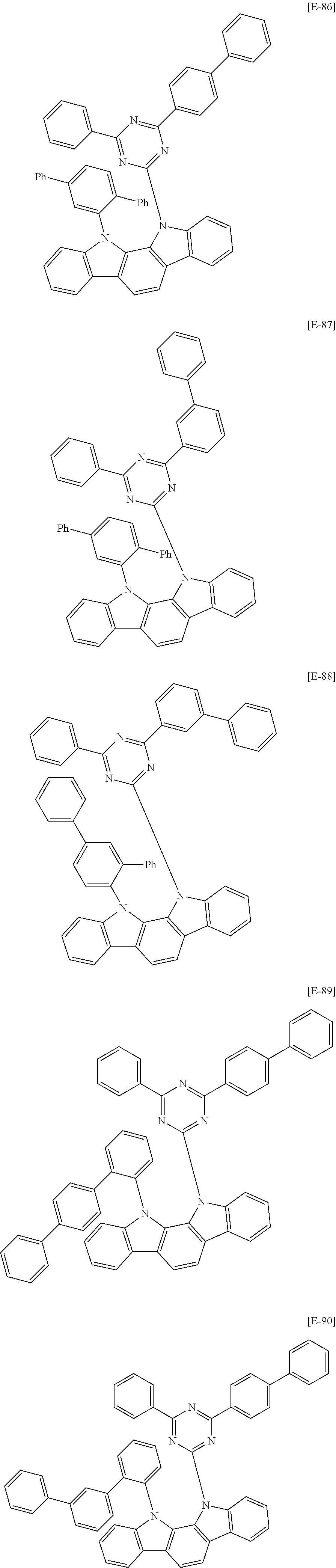

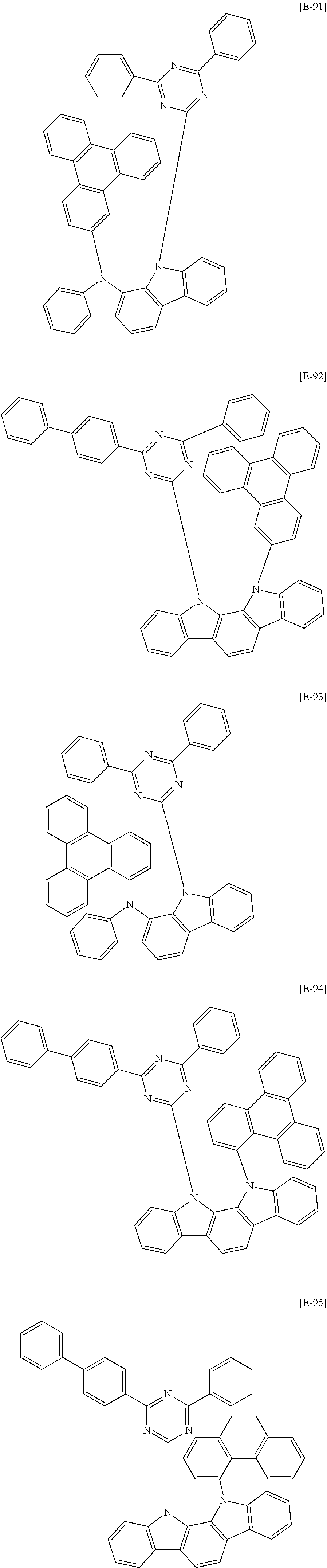 [SMILES: c1ccc(-c2nc(-c3ccccc3)nc(-n3c4ccccc4c4ccc5c6ccccc6n(-c6ccc7c8ccccc8c8ccccc8c7c6)c5c43)n2)cc1 ; c1ccc(-c2ccc(-c3nc(-c4ccccc4)nc(-n4c5ccccc5c5ccc6c7ccccc7n(-c7ccc8c9ccccc9c9ccccc9c8c7)c6c54)n3)cc2)cc1 ; c1ccc(-c2nc(-c3ccccc3)nc(-n3c4ccccc4c4ccc5c6ccccc6n(-c6cccc7c8ccccc8c8ccccc8c67)c5c43)n2)cc1 ; c1ccc(-c2ccc(-c3nc(-c4ccccc4)nc(-n4c5ccccc5c5ccc6c7ccccc7n(-c7cccc8c9ccccc9c9ccccc9c78)c6c54)n3)cc2)cc1 ; c1ccc(-c2ccc(-c3nc(-c4ccccc4)nc(-n4c5ccccc5c5ccc6c7ccccc7n(-c7cccc8ccc9ccccc9c78)c6c54)n3)cc2)cc1]

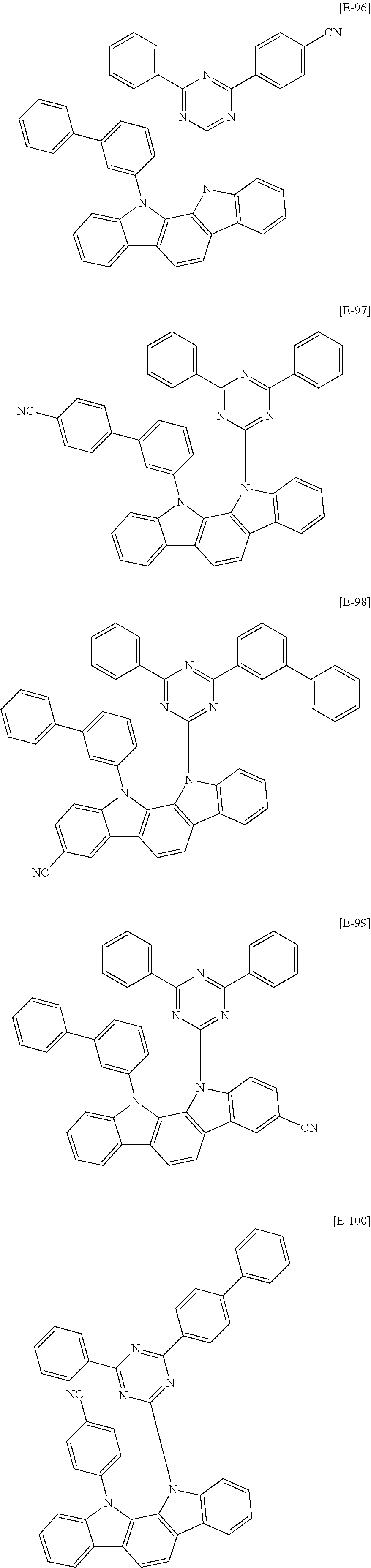 [SMILES: N#Cc1ccc(-c2nc(-c3ccccc3)nc(-n3c4ccccc4c4ccc5c6ccccc6n(-c6cccc(-c7ccccc7)c6)c5c43)n2)cc1 ; N#Cc1ccc(-c2cccc(-n3c4ccccc4c4ccc5c6ccccc6n(-c6nc(-c7ccccc7)nc(-c7ccccc7)n6)c5c43)c2)cc1 ; N#Cc1ccc2c(c1)c1ccc3c4ccccc4n(-c4nc(-c5ccccc5)nc(-c5cccc(-c6ccccc6)c5)n4)c3c1n2-c1cccc(-c2ccccc2)c1 ; N#Cc1ccc2c(c1)c1ccc3c4ccccc4n(-c4cccc(-c5ccccc5)c4)c3c1n2-c1nc(-c2ccccc2)nc(-c2ccccc2)n1 ; N#Cc1ccc(-n2c3ccccc3c3ccc4c5ccccc5n(-c5nc(-c6ccccc6)nc(-c6ccc(-c7ccccc7)cc6)n5)c4c32)cc1]

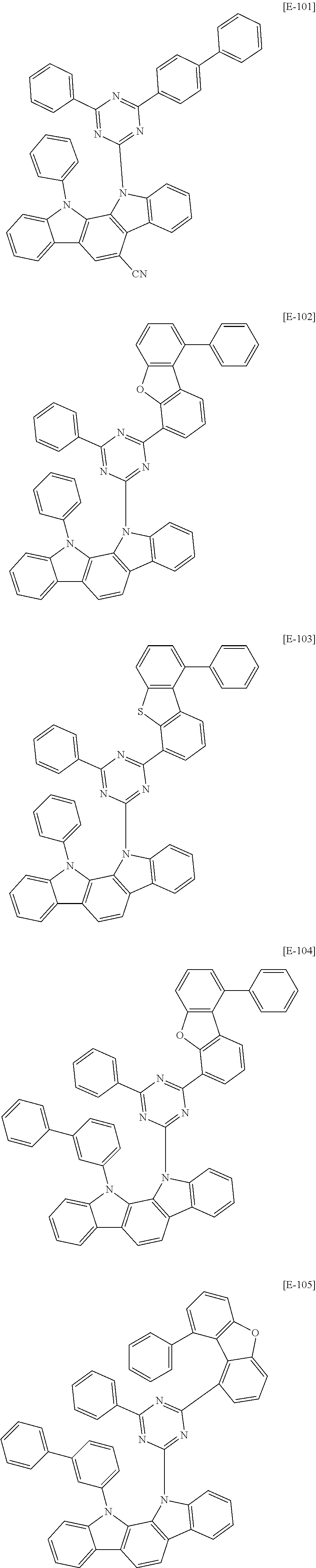

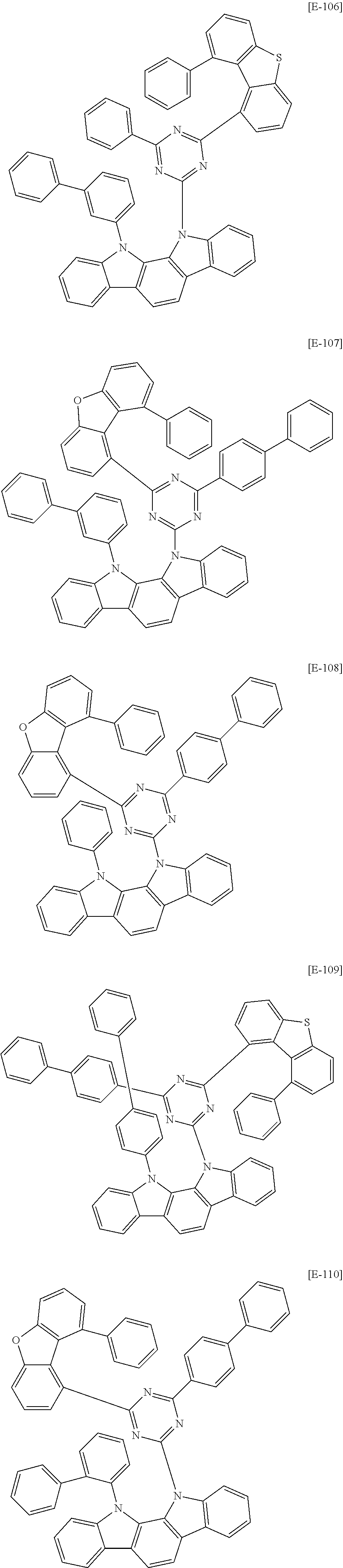

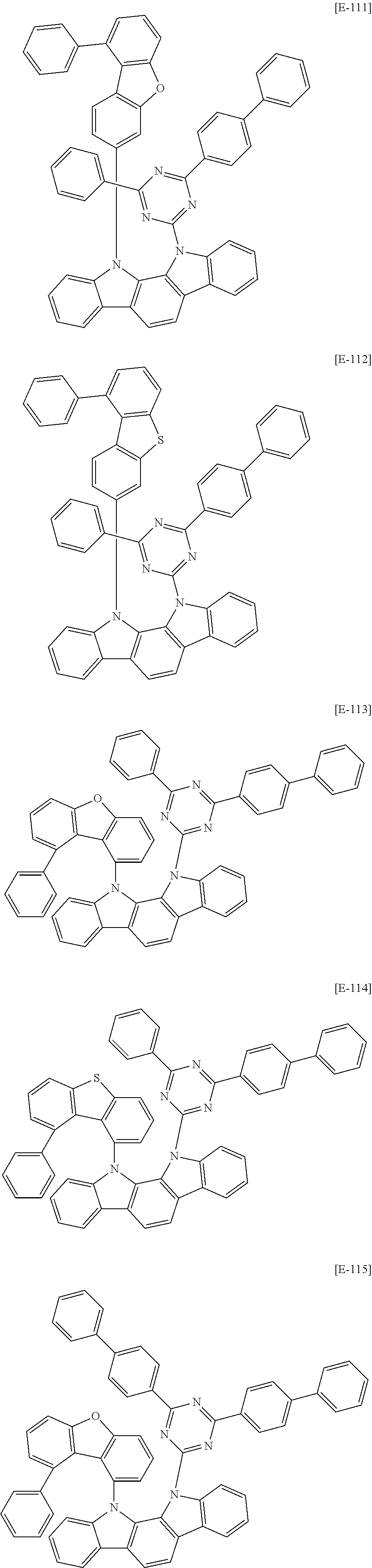

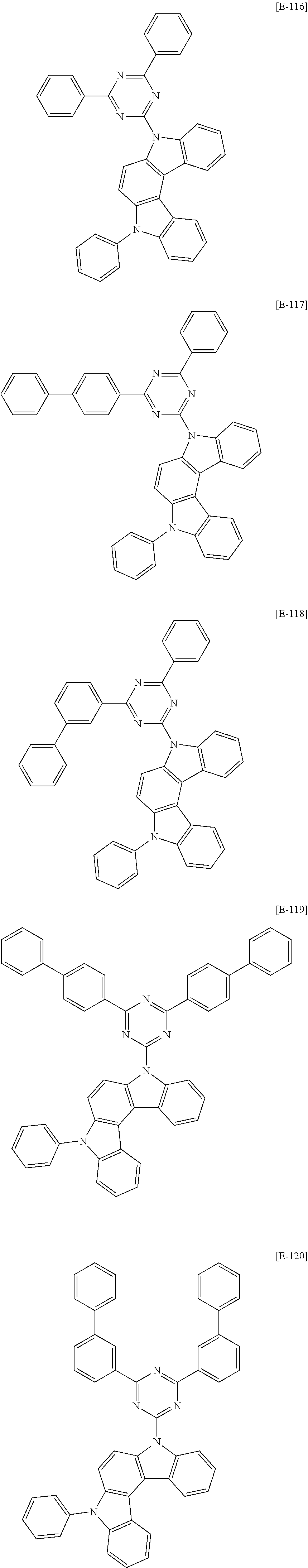 [SMILES: c1ccc(-c2nc(-c3ccccc3)nc(-n3c4ccccc4c4c5c6ccccc6n(-c6ccccc6)c5ccc43)n2)cc1 ; c1ccc(-c2ccc(-c3nc(-c4ccccc4)nc(-n4c5ccccc5c5c6c7ccccc7n(-c7ccccc7)c6ccc54)n3)cc2)cc1 ; c1ccc(-c2cccc(-c3nc(-c4ccccc4)nc(-n4c5ccccc5c5c6c7ccccc7n(-c7ccccc7)c6ccc54)n3)c2)cc1 ; c1ccc(-c2ccc(-c3nc(-c4ccc(-c5ccccc5)cc4)nc(-n4c5ccccc5c5c6c7ccccc7n(-c7ccccc7)c6ccc54)n3)cc2)cc1 ; c1ccc(-c2cccc(-c3nc(-c4cccc(-c5ccccc5)c4)nc(-n4c5ccccc5c5c6c7ccccc7n(-c7ccccc7)c6ccc54)n3)c2)cc1]

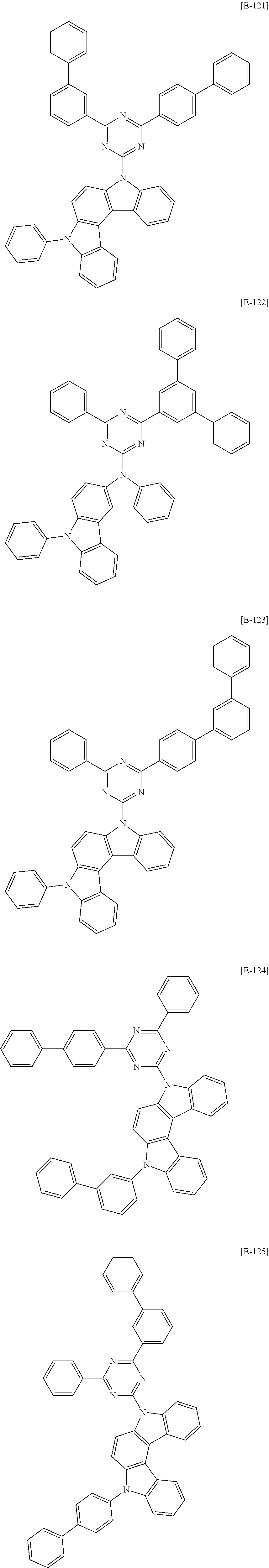 [SMILES: c1ccc(-c2ccc(-c3nc(-c4cccc(-c5ccccc5)c4)nc(-n4c5ccccc5c5c6c7ccccc7n(-c7ccccc7)c6ccc54)n3)cc2)cc1 ; c1ccc(-c2cc(-c3ccccc3)cc(-c3nc(-c4ccccc4)nc(-n4c5ccccc5c5c6c7ccccc7n(-c7ccccc7)c6ccc54)n3)c2)cc1 ; c1ccc(-c2cccc(-c3ccc(-c4nc(-c5ccccc5)nc(-n5c6ccccc6c6c7c8ccccc8n(-c8ccccc8)c7ccc65)n4)cc3)c2)cc1 ; c1ccc(-c2ccc(-c3nc(-c4ccccc4)nc(-n4c5ccccc5c5c6c7ccccc7n(-c7cccc(-c8ccccc8)c7)c6ccc54)n3)cc2)cc1 ; c1ccc(-c2ccc(-n3c4ccccc4c4c5c6ccccc6n(-c6nc(-c7ccccc7)nc(-c7cccc(-c8ccccc8)c7)n6)c5ccc43)cc2)cc1]

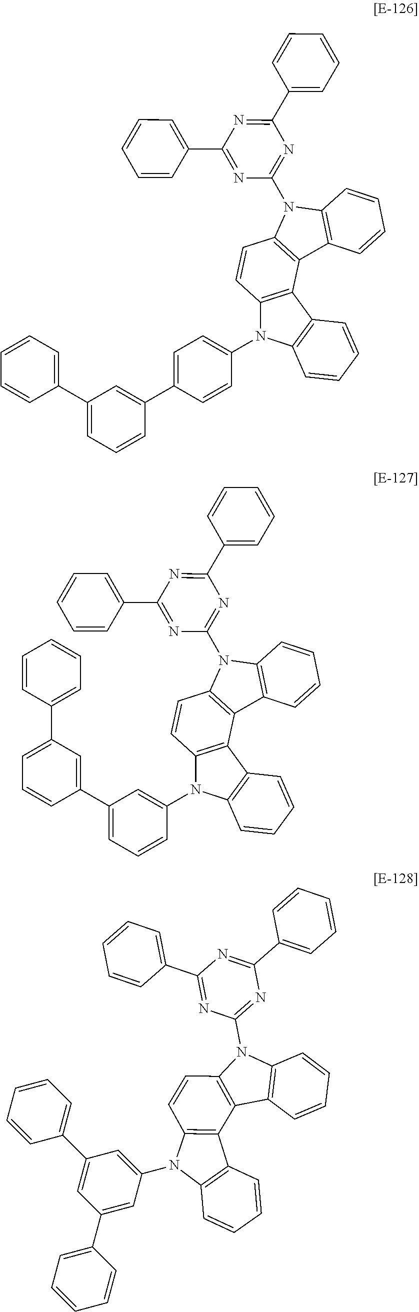 [SMILES: c1ccc(-c2cccc(-c3ccc(-n4c5ccccc5c5c6c7ccccc7n(-c7nc(-c8ccccc8)nc(-c8ccccc8)n7)c6ccc54)cc3)c2)cc1 ; c1ccc(-c2cccc(-c3cccc(-n4c5ccccc5c5c6c7ccccc7n(-c7nc(-c8ccccc8)nc(-c8ccccc8)n7)c6ccc54)c3)c2)cc1 ; c1ccc(-c2cc(-c3ccccc3)cc(-n3c4ccccc4c4c5c6ccccc6n(-c6nc(-c7ccccc7)nc(-c7ccccc7)n6)c5ccc43)c2)cc1]

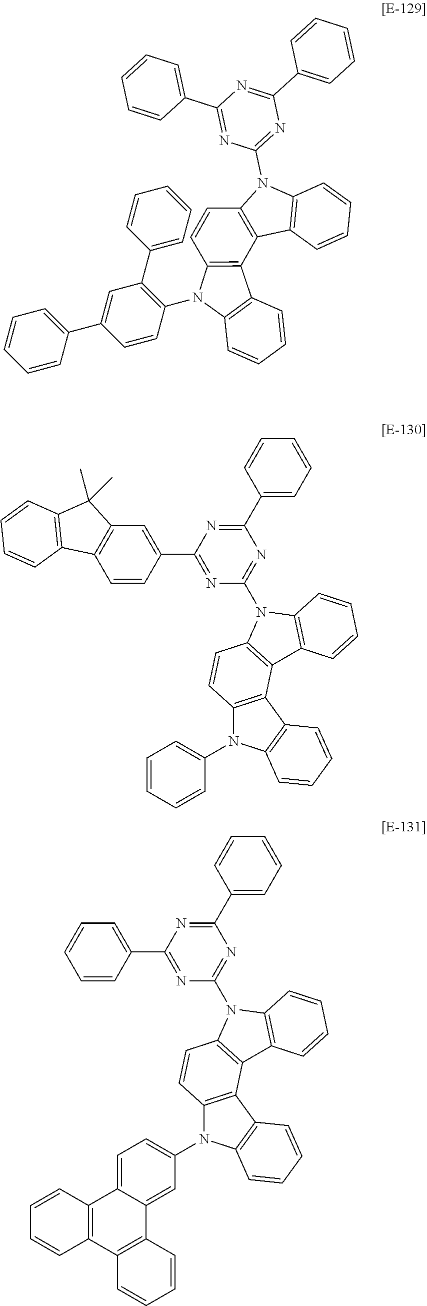 [SMILES: c1ccc(-c2ccc(-n3c4ccccc4c4c5c6ccccc6n(-c6nc(-c7ccccc7)nc(-c7ccccc7)n6)c5ccc43)c(-c3ccccc3)c2)cc1 ; CC1(C)c2ccccc2-c2ccc(-c3nc(-c4ccccc4)nc(-n4c5ccccc5c5c6c7ccccc7n(-c7ccccc7)c6ccc54)n3)cc21 ; c1ccc(-c2nc(-c3ccccc3)nc(-n3c4ccccc4c4c5c6ccccc6n(-c6ccc7c8ccccc8c8ccccc8c7c6)c5ccc43)n2)cc1]

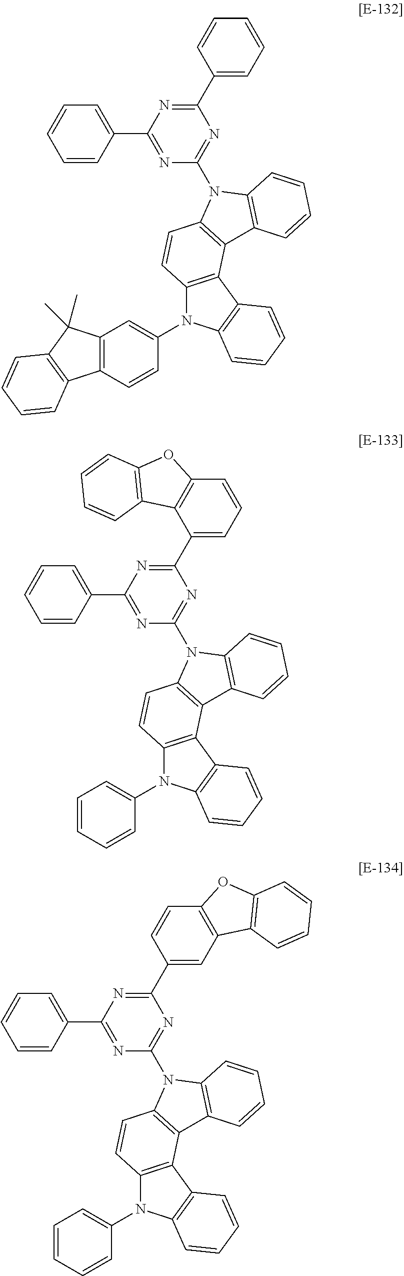 [SMILES: CC1(C)c2ccccc2-c2ccc(-n3c4ccccc4c4c5c6ccccc6n(-c6nc(-c7ccccc7)nc(-c7ccccc7)n6)c5ccc43)cc21 ; c1ccc(-c2nc(-c3cccc4oc5ccccc5c34)nc(-n3c4ccccc4c4c5c6ccccc6n(-c6ccccc6)c5ccc43)n2)cc1 ; c1ccc(-c2nc(-c3ccc4oc5ccccc5c4c3)nc(-n3c4ccccc4c4c5c6ccccc6n(-c6ccccc6)c5ccc43)n2)cc1]

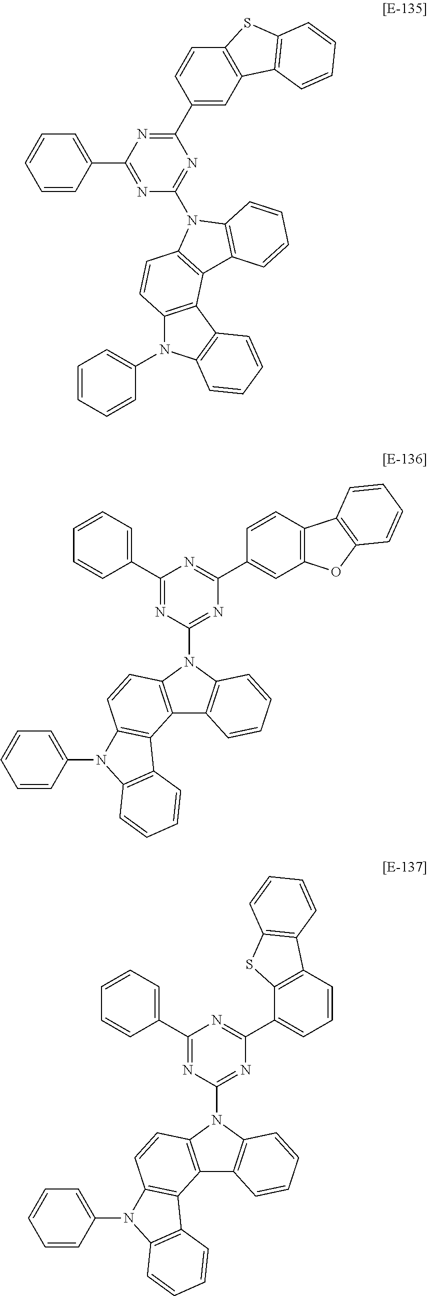 [SMILES: c1ccc(-c2nc(-c3ccc4sc5ccccc5c4c3)nc(-n3c4ccccc4c4c5c6ccccc6n(-c6ccccc6)c5ccc43)n2)cc1 ; c1ccc(-c2nc(-c3ccc4c(c3)oc3ccccc34)nc(-n3c4ccccc4c4c5c6ccccc6n(-c6ccccc6)c5ccc43)n2)cc1 ; c1ccc(-c2nc(-c3cccc4c3sc3ccccc34)nc(-n3c4ccccc4c4c5c6ccccc6n(-c6ccccc6)c5ccc43)n2)cc1]

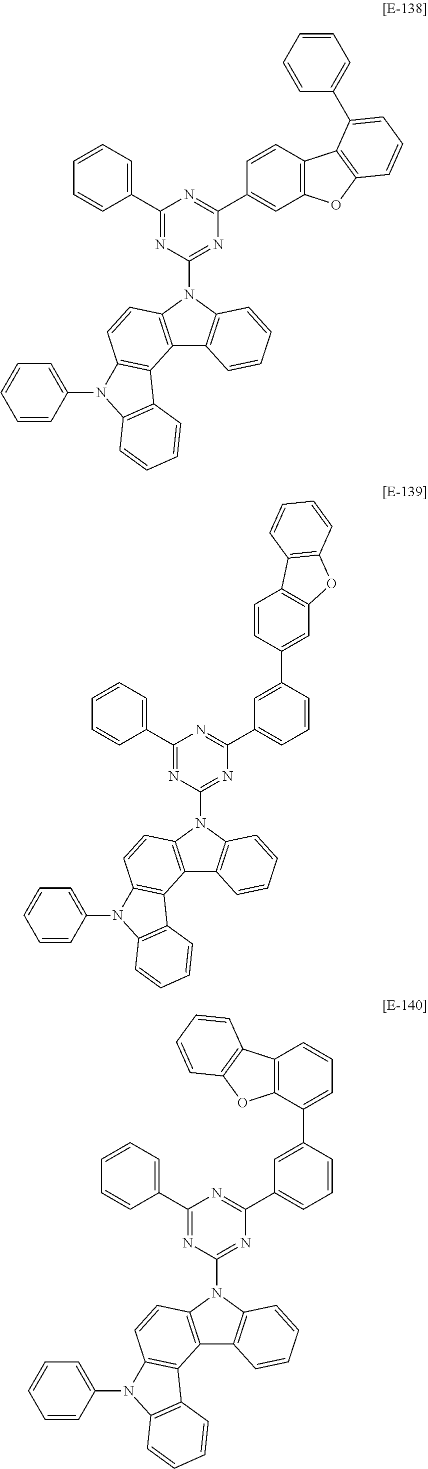

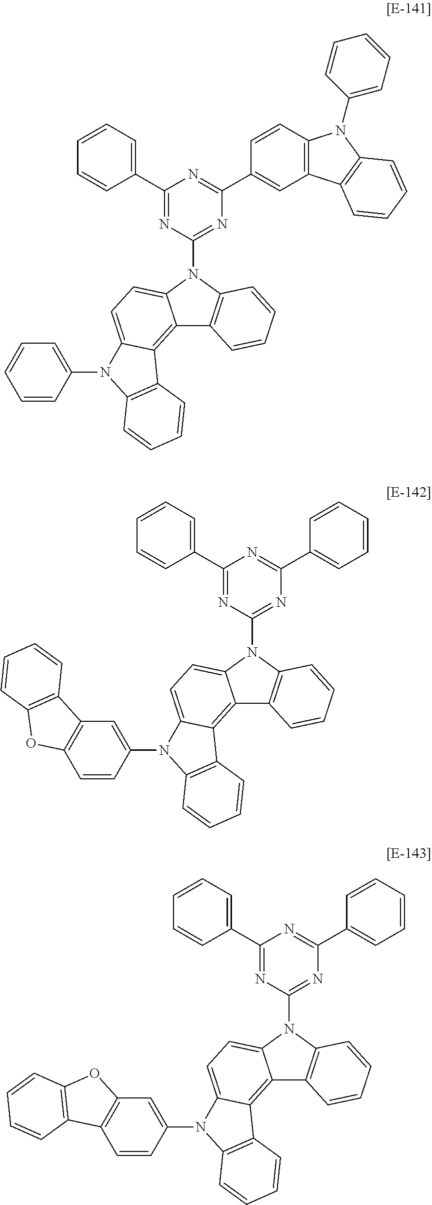 [SMILES: c1ccc(-c2nc(-c3ccc4c(c3)c3ccccc3n4-c3ccccc3)nc(-n3c4ccccc4c4c5c6ccccc6n(-c6ccccc6)c5ccc43)n2)cc1 ; c1ccc(-c2nc(-c3ccccc3)nc(-n3c4ccccc4c4c5c6ccccc6n(-c6ccc7oc8ccccc8c7c6)c5ccc43)n2)cc1 ; c1ccc(-c2nc(-c3ccccc3)nc(-n3c4ccccc4c4c5c6ccccc6n(-c6ccc7c(c6)oc6ccccc67)c5ccc43)n2)cc1]

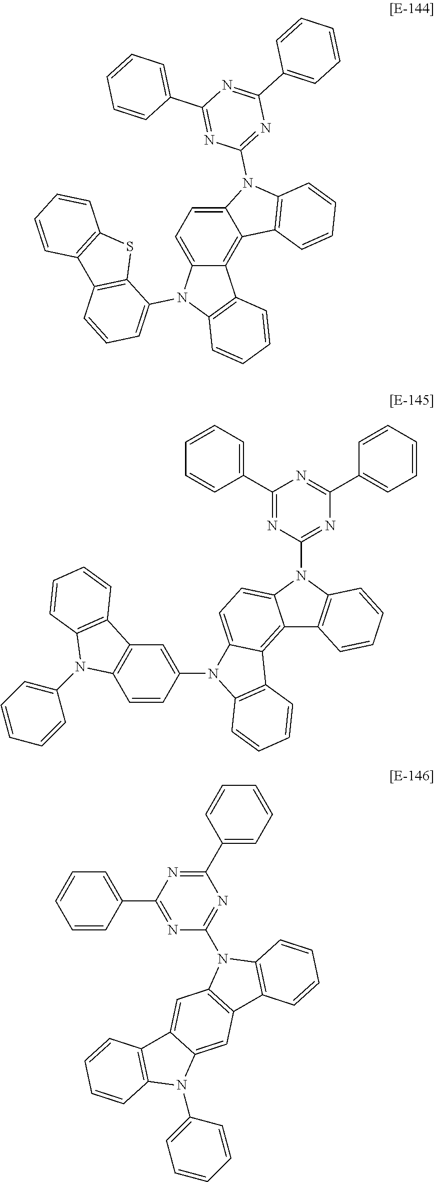

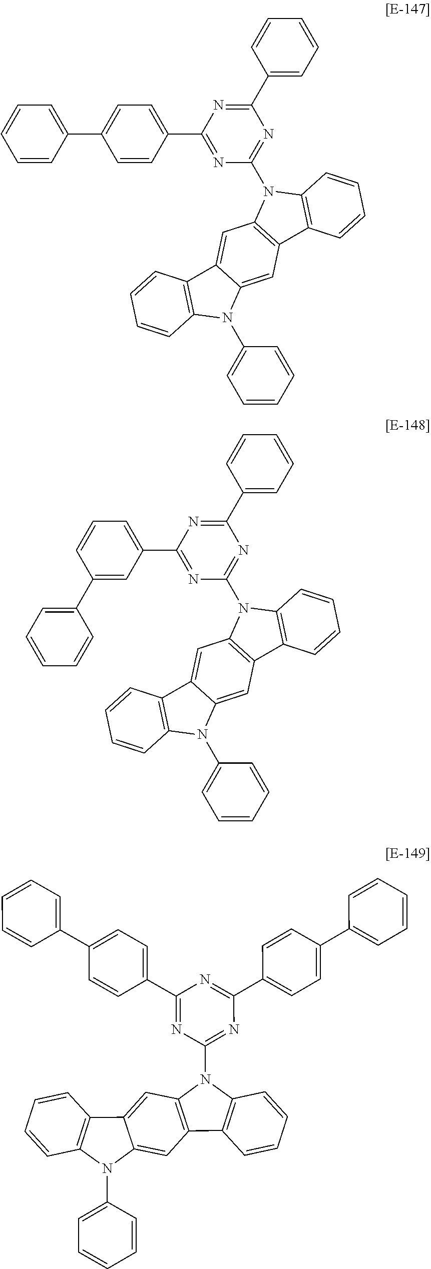

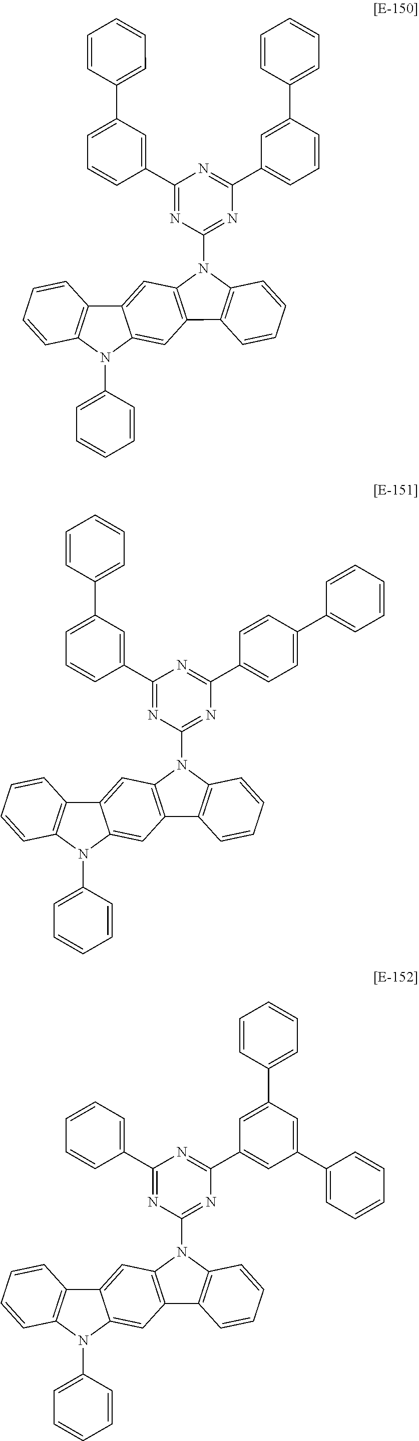 [SMILES: c1ccc(-c2cccc(-c3nc(-c4cccc(-c5ccccc5)c4)nc(-n4c5ccccc5c5cc6c(cc54)c4ccccc4n6-c4ccccc4)n3)c2)cc1 ; c1ccc(-c2ccc(-c3nc(-c4cccc(-c5ccccc5)c4)nc(-n4c5ccccc5c5cc6c(cc54)c4ccccc4n6-c4ccccc4)n3)cc2)cc1 ; c1ccc(-c2cc(-c3ccccc3)cc(-c3nc(-c4ccccc4)nc(-n4c5ccccc5c5cc6c(cc54)c4ccccc4n6-c4ccccc4)n3)c2)cc1]

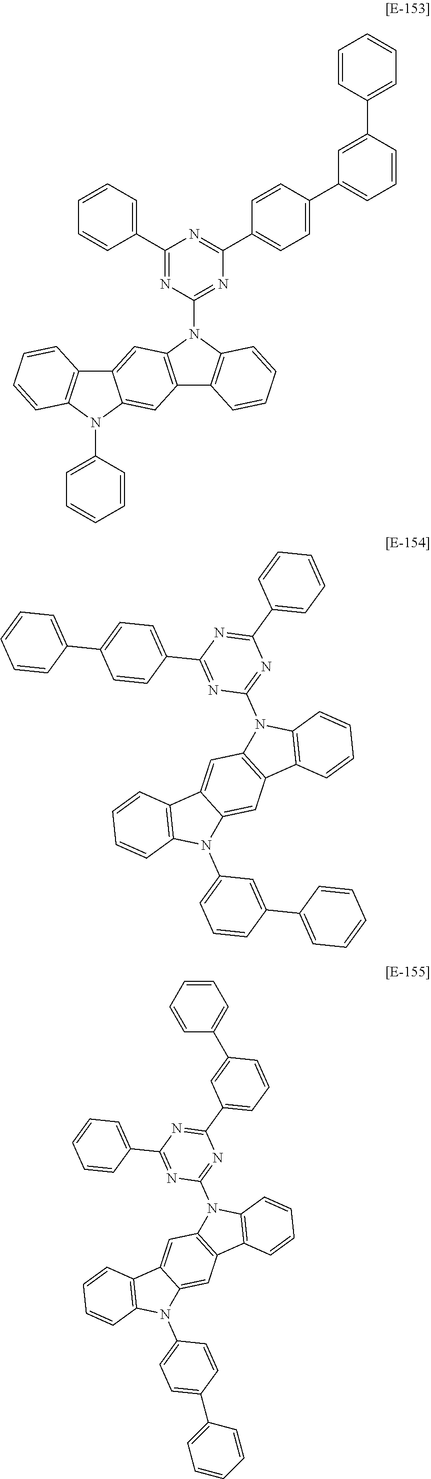 [SMILES: c1ccc(-c2cccc(-c3ccc(-c4nc(-c5ccccc5)nc(-n5c6ccccc6c6cc7c(cc65)c5ccccc5n7-c5ccccc5)n4)cc3)c2)cc1 ; c1ccc(-c2ccc(-c3nc(-c4ccccc4)nc(-n4c5ccccc5c5cc6c(cc54)c4ccccc4n6-c4cccc(-c5ccccc5)c4)n3)cc2)cc1 ; c1ccc(-c2ccc(-n3c4ccccc4c4cc5c(cc43)c3ccccc3n5-c3nc(-c4ccccc4)nc(-c4cccc(-c5ccccc5)c4)n3)cc2)cc1]

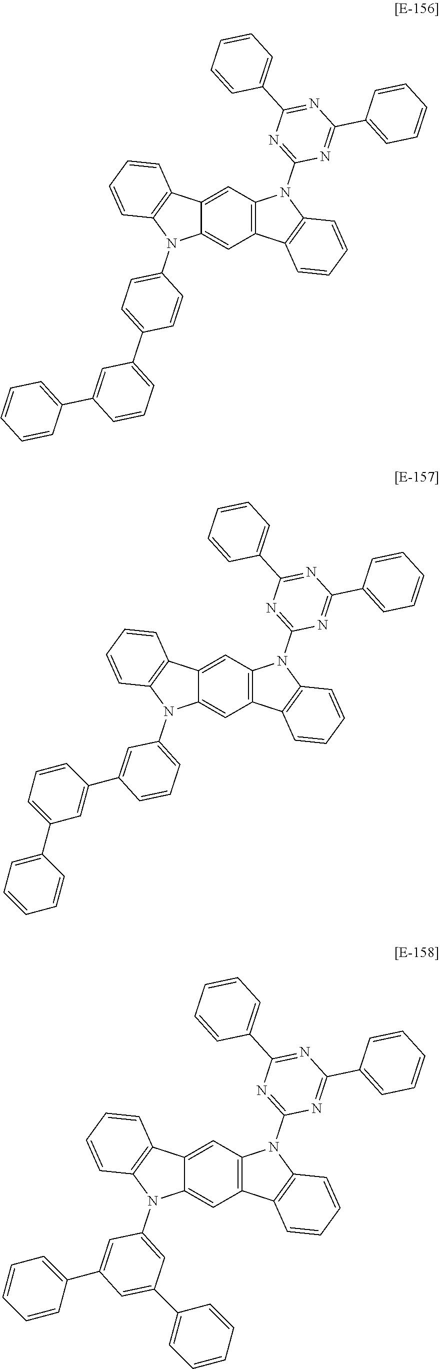 [SMILES: c1ccc(-c2cccc(-c3ccc(-n4c5ccccc5c5cc6c(cc54)c4ccccc4n6-c4nc(-c5ccccc5)nc(-c5ccccc5)n4)cc3)c2)cc1 ; c1ccc(-c2cccc(-c3cccc(-n4c5ccccc5c5cc6c(cc54)c4ccccc4n6-c4nc(-c5ccccc5)nc(-c5ccccc5)n4)c3)c2)cc1 ; c1ccc(-c2cc(-c3ccccc3)cc(-n3c4ccccc4c4cc5c(cc43)c3ccccc3n5-c3nc(-c4ccccc4)nc(-c4ccccc4)n3)c2)cc1]

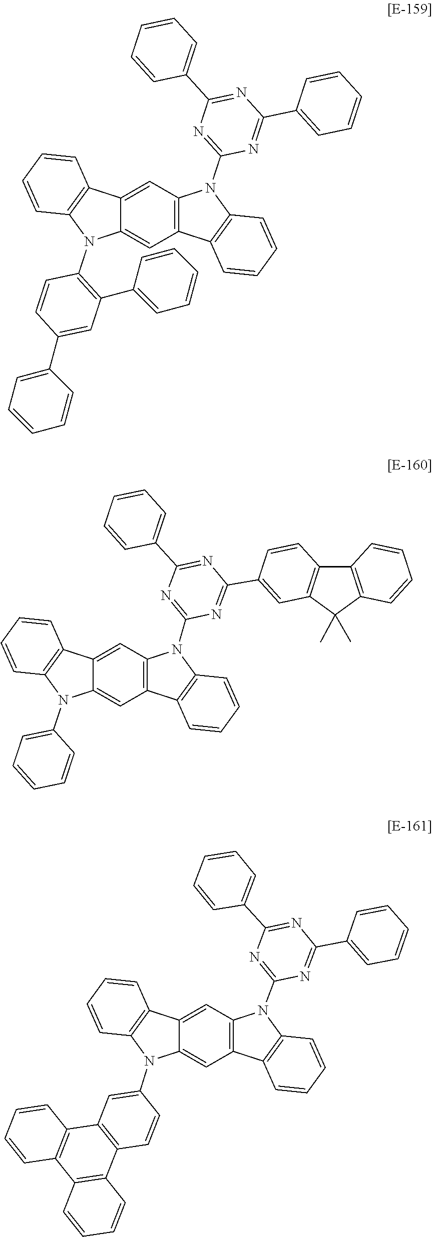 [SMILES: c1ccc(-c2ccc(-n3c4ccccc4c4cc5c(cc43)c3ccccc3n5-c3nc(-c4ccccc4)nc(-c4ccccc4)n3)c(-c3ccccc3)c2)cc1 ; CC1(C)c2ccccc2-c2ccc(-c3nc(-c4ccccc4)nc(-n4c5ccccc5c5cc6c(cc54)c4ccccc4n6-c4ccccc4)n3)cc21 ; c1ccc(-c2nc(-c3ccccc3)nc(-n3c4ccccc4c4cc5c(cc43)c3ccccc3n5-c3ccc4c5ccccc5c5ccccc5c4c3)n2)cc1]

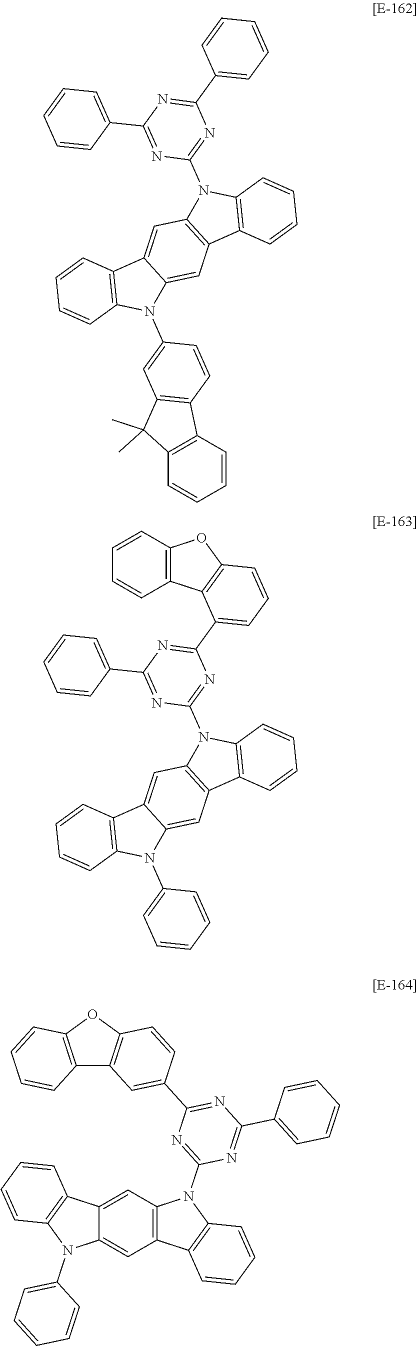

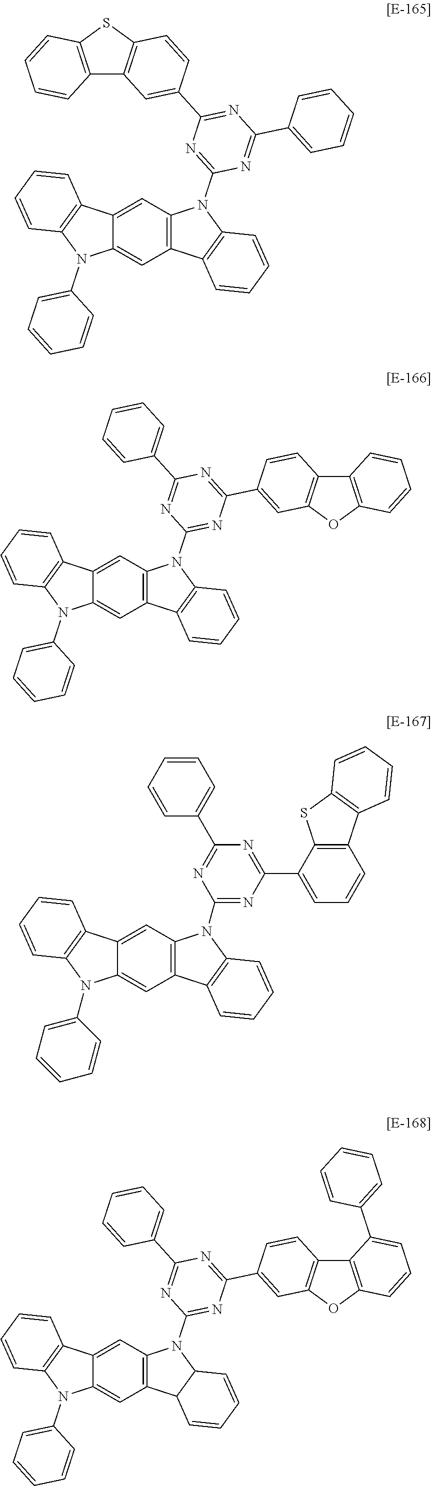 [SMILES: c1ccc(-c2nc(-c3ccc4sc5ccccc5c4c3)nc(-n3c4ccccc4c4cc5c(cc43)c3ccccc3n5-c3ccccc3)n2)cc1 ; c1ccc(-c2nc(-c3ccc4c(c3)oc3ccccc34)nc(-n3c4ccccc4c4cc5c(cc43)c3ccccc3n5-c3ccccc3)n2)cc1 ; c1ccc(-c2nc(-c3cccc4c3sc3ccccc34)nc(-n3c4ccccc4c4cc5c(cc43)c3ccccc3n5-c3ccccc3)n2)cc1 ; C1=CC2c3cc4c(cc3N(c3nc(-c5ccccc5)nc(-c5ccc6c(c5)oc5cccc(-c7ccccc7)c56)n3)C2C=C1)c1ccccc1n4-c1ccccc1]

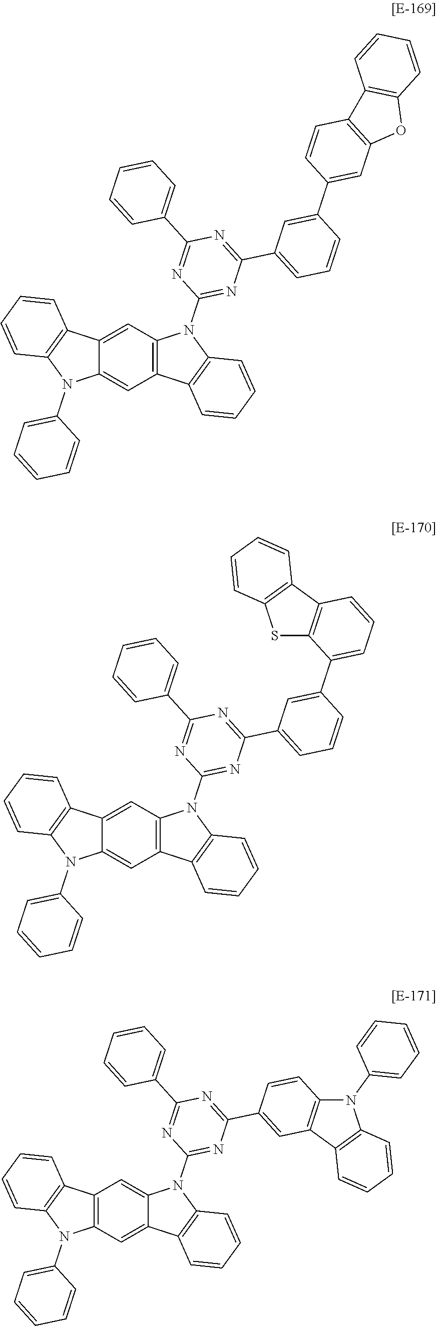 [SMILES: c1ccc(-c2nc(-c3cccc(-c4ccc5c(c4)oc4ccccc45)c3)nc(-n3c4ccccc4c4cc5c(cc43)c3ccccc3n5-c3ccccc3)n2)cc1 ; c1ccc(-c2nc(-c3cccc(-c4cccc5c4sc4ccccc45)c3)nc(-n3c4ccccc4c4cc5c(cc43)c3ccccc3n5-c3ccccc3)n2)cc1 ; c1ccc(-c2nc(-c3ccc4c(c3)c3ccccc3n4-c3ccccc3)nc(-n3c4ccccc4c4cc5c(cc43)c3ccccc3n5-c3ccccc3)n2)cc1]

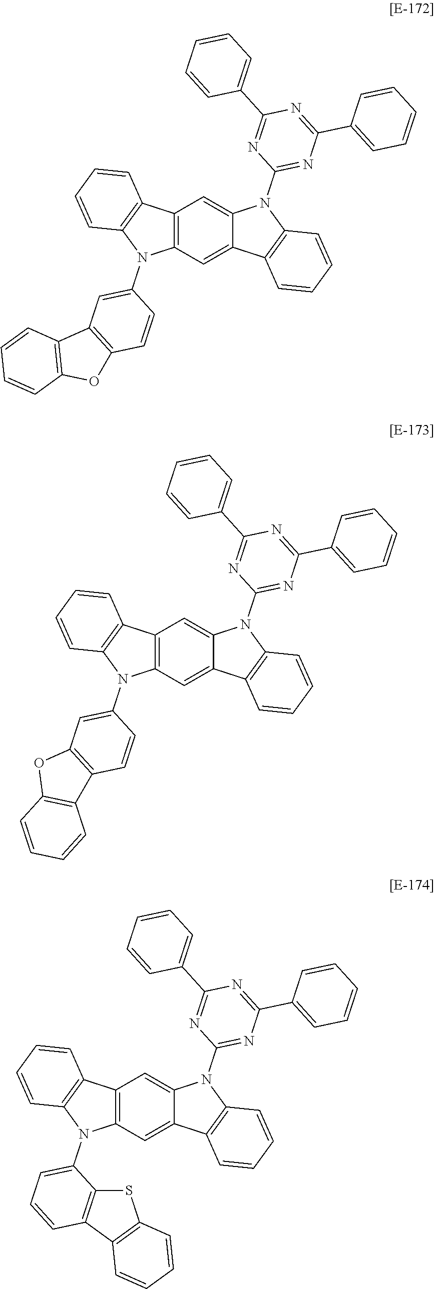 [SMILES: c1ccc(-c2nc(-c3ccccc3)nc(-n3c4ccccc4c4cc5c(cc43)c3ccccc3n5-c3ccc4oc5ccccc5c4c3)n2)cc1 ; c1ccc(-c2nc(-c3ccccc3)nc(-n3c4ccccc4c4cc5c(cc43)c3ccccc3n5-c3ccc4c(c3)oc3ccccc34)n2)cc1 ; c1ccc(-c2nc(-c3ccccc3)nc(-n3c4ccccc4c4cc5c(cc43)c3ccccc3n5-c3cccc4c3sc3ccccc34)n2)cc1]

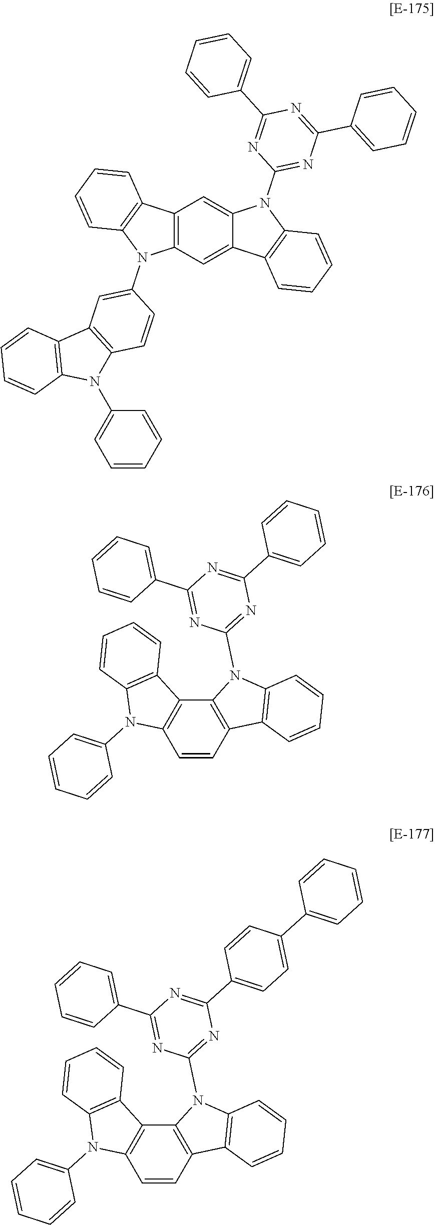 [SMILES: c1ccc(-c2nc(-c3ccccc3)nc(-n3c4ccccc4c4cc5c(cc43)c3ccccc3n5-c3ccc4c(c3)c3ccccc3n4-c3ccccc3)n2)cc1 ; c1ccc(-c2nc(-c3ccccc3)nc(-n3c4ccccc4c4ccc5c(c6ccccc6n5-c5ccccc5)c43)n2)cc1 ; c1ccc(-c2ccc(-c3nc(-c4ccccc4)nc(-n4c5ccccc5c5ccc6c(c7ccccc7n6-c6ccccc6)c54)n3)cc2)cc1]

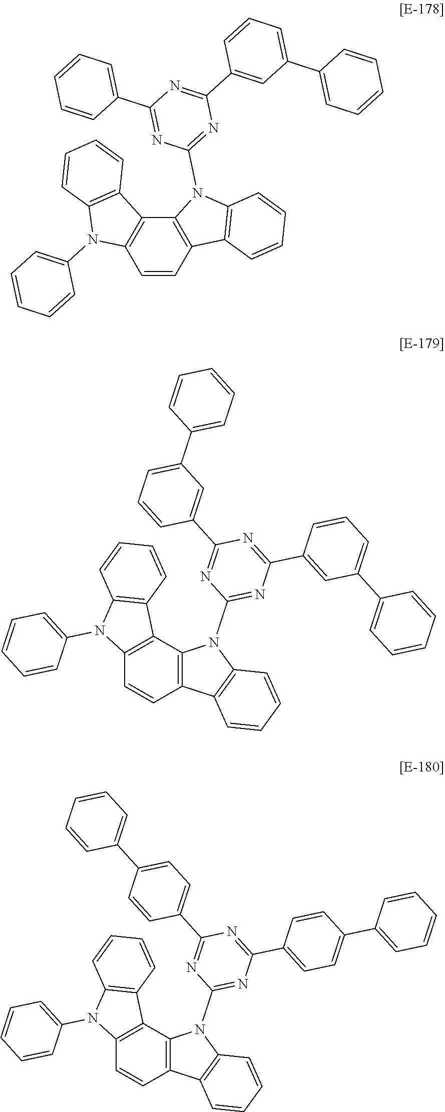 [SMILES: c1ccc(-c2cccc(-c3nc(-c4ccccc4)nc(-n4c5ccccc5c5ccc6c(c7ccccc7n6-c6ccccc6)c54)n3)c2)cc1 ; c1ccc(-c2cccc(-c3nc(-c4cccc(-c5ccccc5)c4)nc(-n4c5ccccc5c5ccc6c(c7ccccc7n6-c6ccccc6)c54)n3)c2)cc1 ; c1ccc(-c2ccc(-c3nc(-c4ccc(-c5ccccc5)cc4)nc(-n4c5ccccc5c5ccc6c(c7ccccc7n6-c6ccccc6)c54)n3)cc2)cc1]

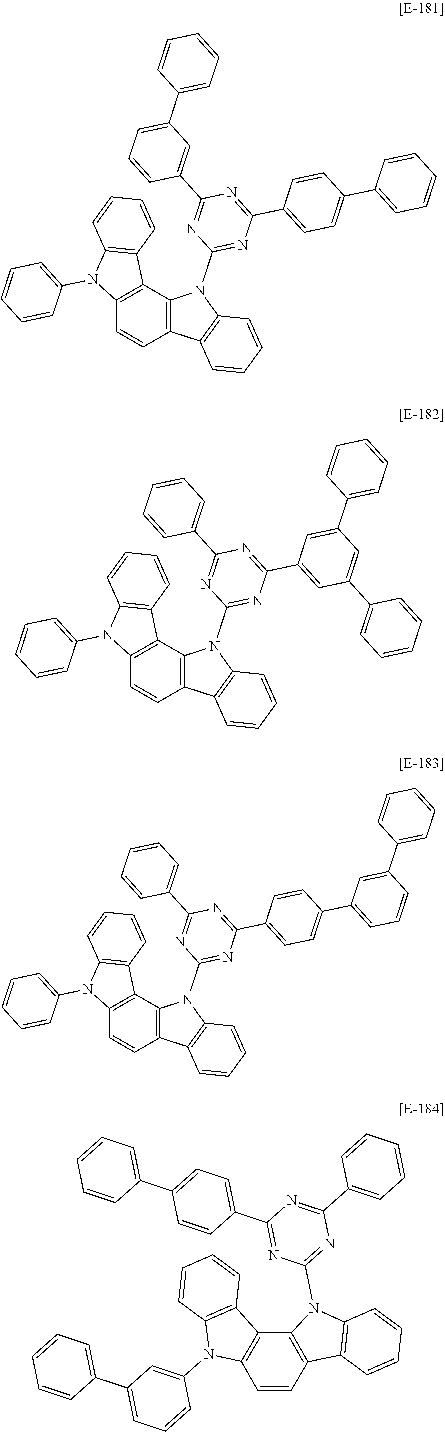 [SMILES: c1ccc(-c2ccc(-c3nc(-c4cccc(-c5ccccc5)c4)nc(-n4c5ccccc5c5ccc6c(c7ccccc7n6-c6ccccc6)c54)n3)cc2)cc1 ; c1ccc(-c2cc(-c3ccccc3)cc(-c3nc(-c4ccccc4)nc(-n4c5ccccc5c5ccc6c(c7ccccc7n6-c6ccccc6)c54)n3)c2)cc1 ; c1ccc(-c2cccc(-c3ccc(-c4nc(-c5ccccc5)nc(-n5c6ccccc6c6ccc7c(c8ccccc8n7-c7ccccc7)c65)n4)cc3)c2)cc1 ; c1ccc(-c2ccc(-c3nc(-c4ccccc4)nc(-n4c5ccccc5c5ccc6c(c7ccccc7n6-c6cccc(-c7ccccc7)c6)c54)n3)cc2)cc1]

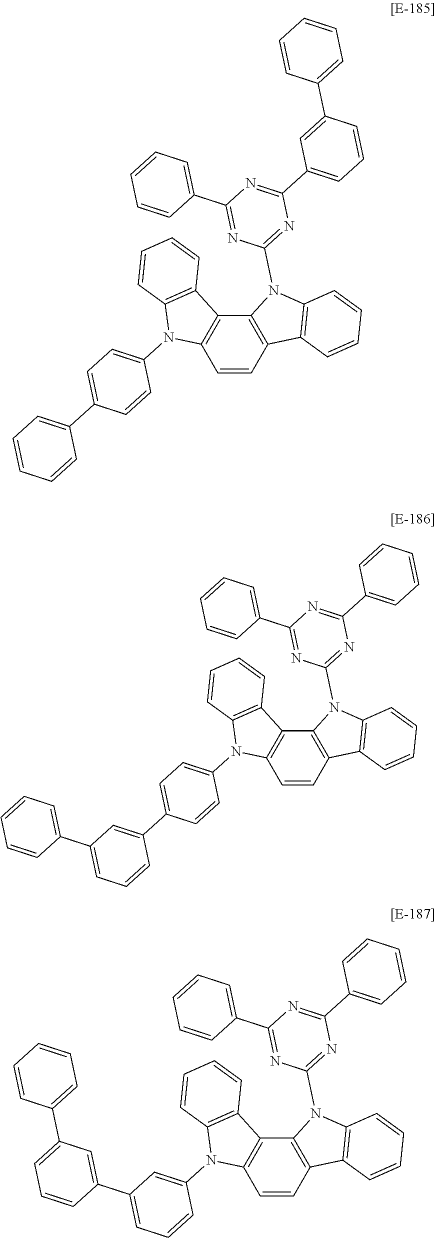 [SMILES: c1ccc(-c2ccc(-n3c4ccccc4c4c3ccc3c5ccccc5n(-c5nc(-c6ccccc6)nc(-c6cccc(-c7ccccc7)c6)n5)c34)cc2)cc1 ; c1ccc(-c2cccc(-c3ccc(-n4c5ccccc5c5c4ccc4c6ccccc6n(-c6nc(-c7ccccc7)nc(-c7ccccc7)n6)c45)cc3)c2)cc1 ; c1ccc(-c2cccc(-c3cccc(-n4c5ccccc5c5c4ccc4c6ccccc6n(-c6nc(-c7ccccc7)nc(-c7ccccc7)n6)c45)c3)c2)cc1]

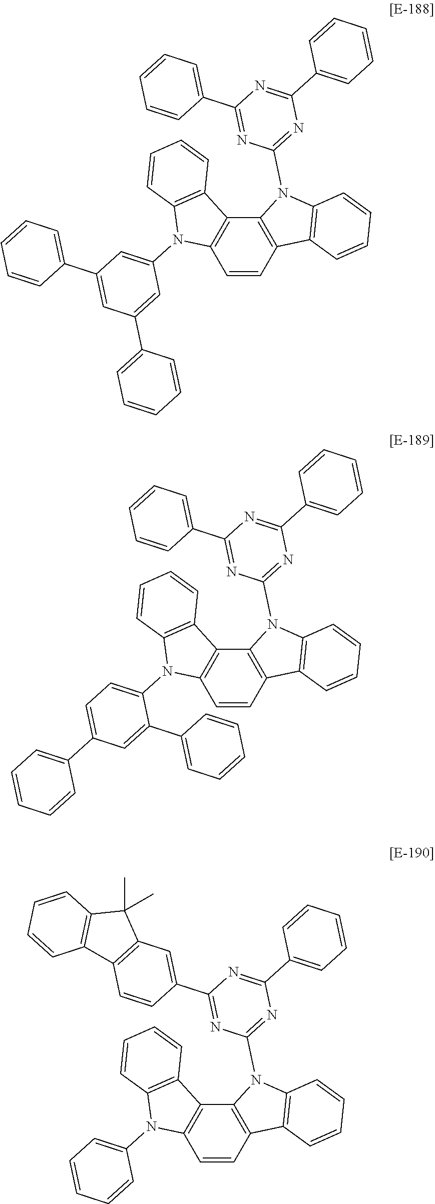 [SMILES: c1ccc(-c2cc(-c3ccccc3)cc(-n3c4ccccc4c4c3ccc3c5ccccc5n(-c5nc(-c6ccccc6)nc(-c6ccccc6)n5)c34)c2)cc1 ; c1ccc(-c2ccc(-n3c4ccccc4c4c3ccc3c5ccccc5n(-c5nc(-c6ccccc6)nc(-c6ccccc6)n5)c34)c(-c3ccccc3)c2)cc1 ; CC1(C)c2ccccc2-c2ccc(-c3nc(-c4ccccc4)nc(-n4c5ccccc5c5ccc6c(c7ccccc7n6-c6ccccc6)c54)n3)cc21]

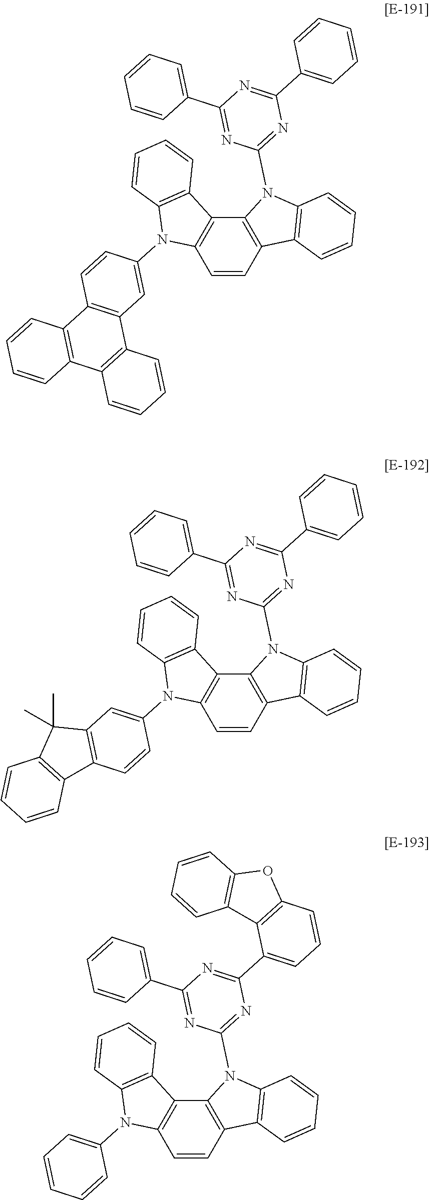 [SMILES: c1ccc(-c2nc(-c3ccccc3)nc(-n3c4ccccc4c4ccc5c(c6ccccc6n5-c5ccc6c7ccccc7c7ccccc7c6c5)c43)n2)cc1 ; CC1(C)c2ccccc2-c2ccc(-n3c4ccccc4c4c3ccc3c5ccccc5n(-c5nc(-c6ccccc6)nc(-c6ccccc6)n5)c34)cc21 ; c1ccc(-c2nc(-c3cccc4oc5ccccc5c34)nc(-n3c4ccccc4c4ccc5c(c6ccccc6n5-c5ccccc5)c43)n2)cc1]

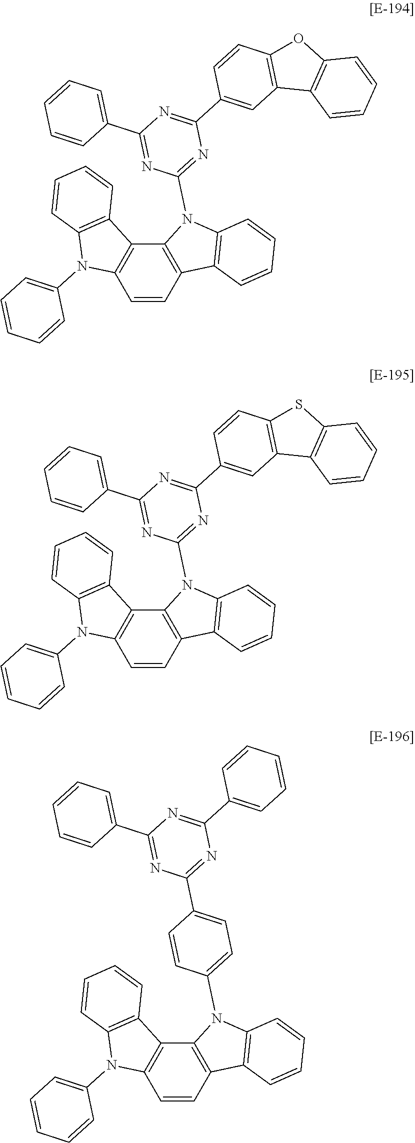 [SMILES: c1ccc(-c2nc(-c3ccc4oc5ccccc5c4c3)nc(-n3c4ccccc4c4ccc5c(c6ccccc6n5-c5ccccc5)c43)n2)cc1 ; c1ccc(-c2nc(-c3ccc4sc5ccccc5c4c3)nc(-n3c4ccccc4c4ccc5c(c6ccccc6n5-c5ccccc5)c43)n2)cc1 ; c1ccc(-c2nc(-c3ccccc3)nc(-c3ccc(-n4c5ccccc5c5ccc6c(c7ccccc7n6-c6ccccc6)c54)cc3)n2)cc1]

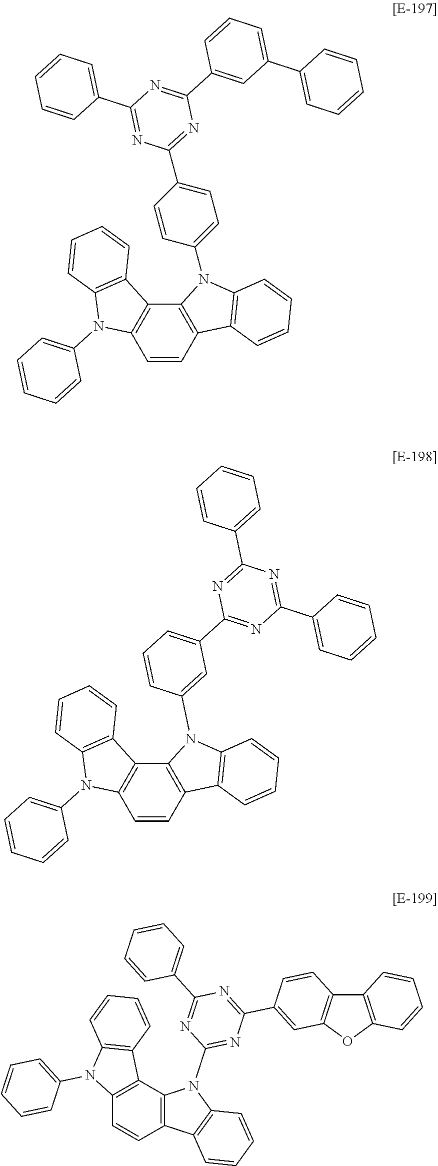 [SMILES: c1ccc(-c2cccc(-c3nc(-c4ccccc4)nc(-c4ccc(-n5c6ccccc6c6ccc7c(c8ccccc8n7-c7ccccc7)c65)cc4)n3)c2)cc1 ; c1ccc(-c2nc(-c3ccccc3)nc(-c3cccc(-n4c5ccccc5c5ccc6c(c7ccccc7n6-c6ccccc6)c54)c3)n2)cc1 ; c1ccc(-c2nc(-c3ccc4c(c3)oc3ccccc34)nc(-n3c4ccccc4c4ccc5c(c6ccccc6n5-c5ccccc5)c43)n2)cc1]

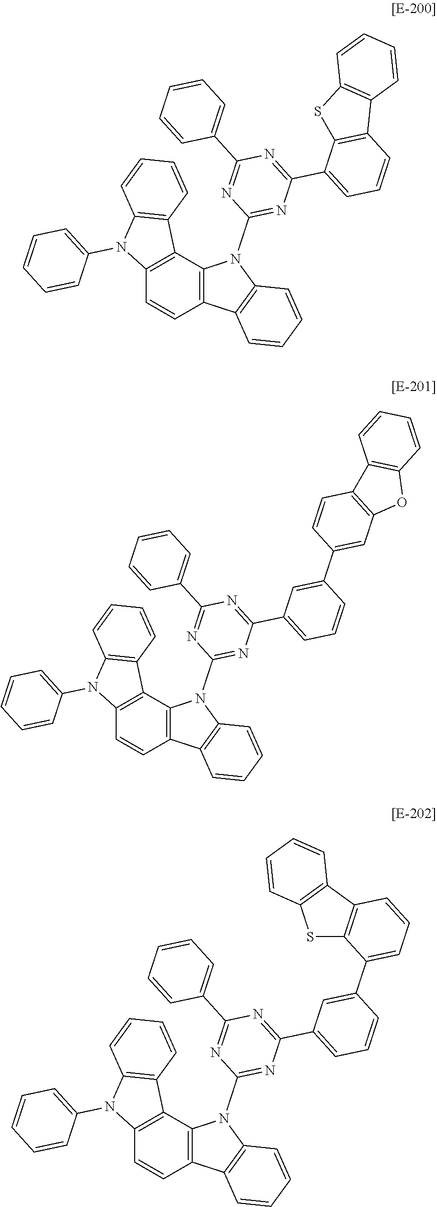 [SMILES: c1ccc(-c2nc(-c3cccc4c3sc3ccccc34)nc(-n3c4ccccc4c4ccc5c(c6ccccc6n5-c5ccccc5)c43)n2)cc1 ; c1ccc(-c2nc(-c3cccc(-c4ccc5c(c4)oc4ccccc45)c3)nc(-n3c4ccccc4c4ccc5c(c6ccccc6n5-c5ccccc5)c43)n2)cc1 ; c1ccc(-c2nc(-c3cccc(-c4cccc5c4sc4ccccc45)c3)nc(-n3c4ccccc4c4ccc5c(c6ccccc6n5-c5ccccc5)c43)n2)cc1]

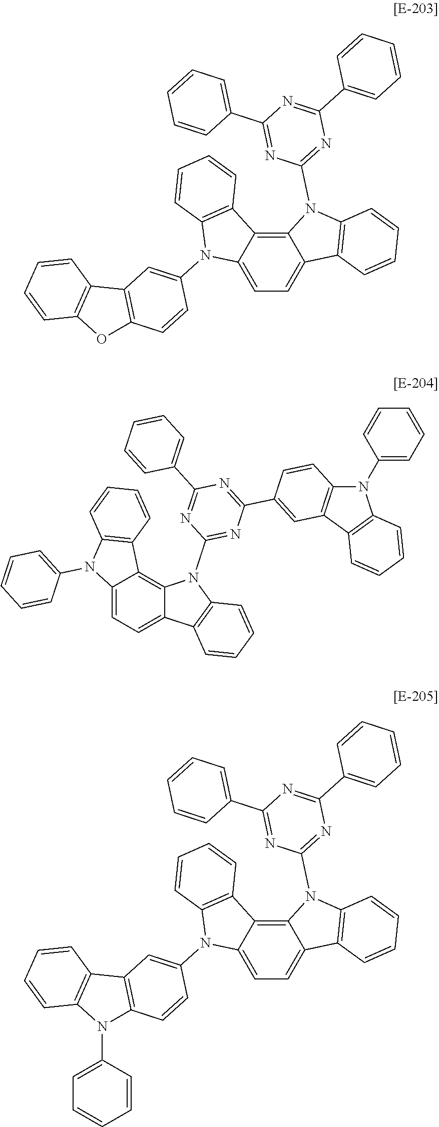 [SMILES: c1ccc(-c2nc(-c3ccccc3)nc(-n3c4ccccc4c4ccc5c(c6ccccc6n5-c5ccc6oc7ccccc7c6c5)c43)n2)cc1 ; c1ccc(-c2nc(-c3ccc4c(c3)c3ccccc3n4-c3ccccc3)nc(-n3c4ccccc4c4ccc5c(c6ccccc6n5-c5ccccc5)c43)n2)cc1 ; c1ccc(-c2nc(-c3ccccc3)nc(-n3c4ccccc4c4ccc5c(c6ccccc6n5-c5ccc6c(c5)c5ccccc5n6-c5ccccc5)c43)n2)cc1]

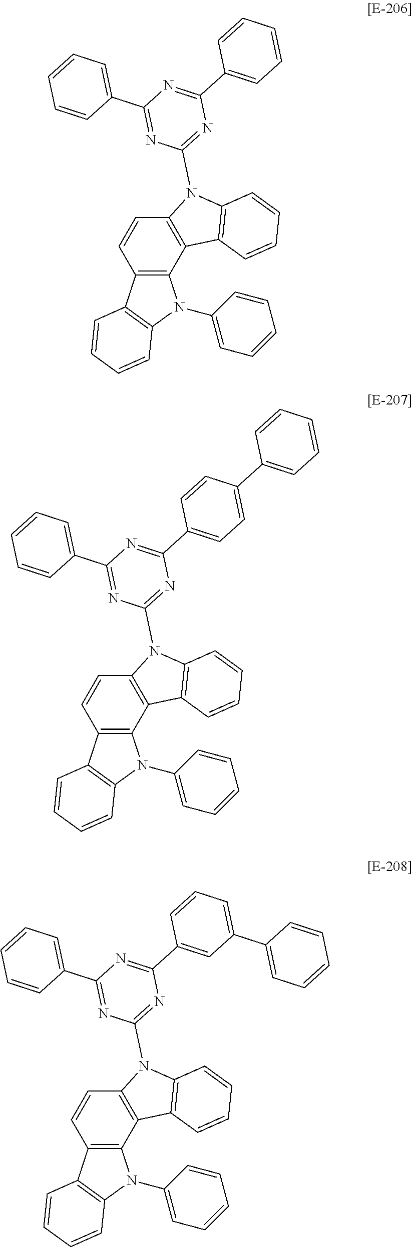 [SMILES: c1ccc(-c2nc(-c3ccccc3)nc(-n3c4ccccc4c4c3ccc3c5ccccc5n(-c5ccccc5)c34)n2)cc1 ; c1ccc(-c2ccc(-c3nc(-c4ccccc4)nc(-n4c5ccccc5c5c4ccc4c6ccccc6n(-c6ccccc6)c45)n3)cc2)cc1 ; c1ccc(-c2cccc(-c3nc(-c4ccccc4)nc(-n4c5ccccc5c5c4ccc4c6ccccc6n(-c6ccccc6)c45)n3)c2)cc1]

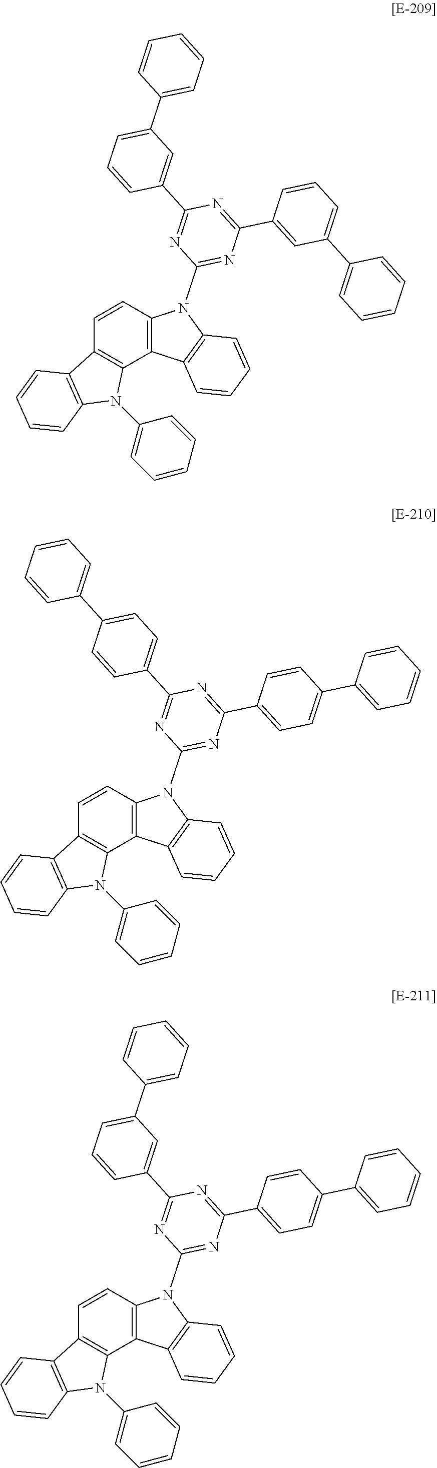 [SMILES: c1ccc(-c2cccc(-c3nc(-c4cccc(-c5ccccc5)c4)nc(-n4c5ccccc5c5c4ccc4c6ccccc6n(-c6ccccc6)c45)n3)c2)cc1 ; c1ccc(-c2ccc(-c3nc(-c4ccc(-c5ccccc5)cc4)nc(-n4c5ccccc5c5c4ccc4c6ccccc6n(-c6ccccc6)c45)n3)cc2)cc1 ; c1ccc(-c2ccc(-c3nc(-c4cccc(-c5ccccc5)c4)nc(-n4c5ccccc5c5c4ccc4c6ccccc6n(-c6ccccc6)c45)n3)cc2)cc1]

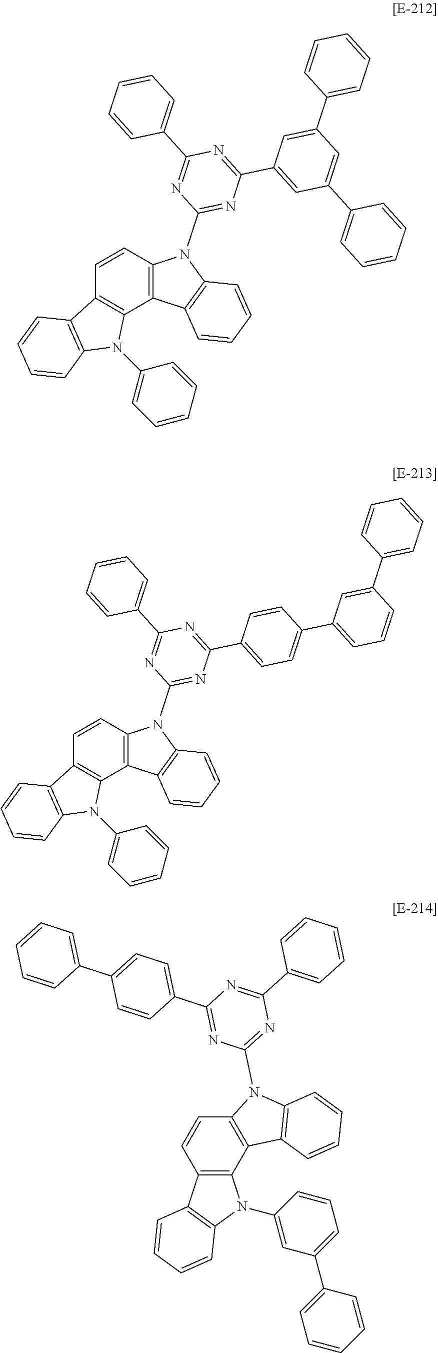 [SMILES: c1ccc(-c2cc(-c3ccccc3)cc(-c3nc(-c4ccccc4)nc(-n4c5ccccc5c5c4ccc4c6ccccc6n(-c6ccccc6)c45)n3)c2)cc1 ; c1ccc(-c2cccc(-c3ccc(-c4nc(-c5ccccc5)nc(-n5c6ccccc6c6c5ccc5c7ccccc7n(-c7ccccc7)c56)n4)cc3)c2)cc1 ; c1ccc(-c2ccc(-c3nc(-c4ccccc4)nc(-n4c5ccccc5c5c4ccc4c6ccccc6n(-c6cccc(-c7ccccc7)c6)c45)n3)cc2)cc1]

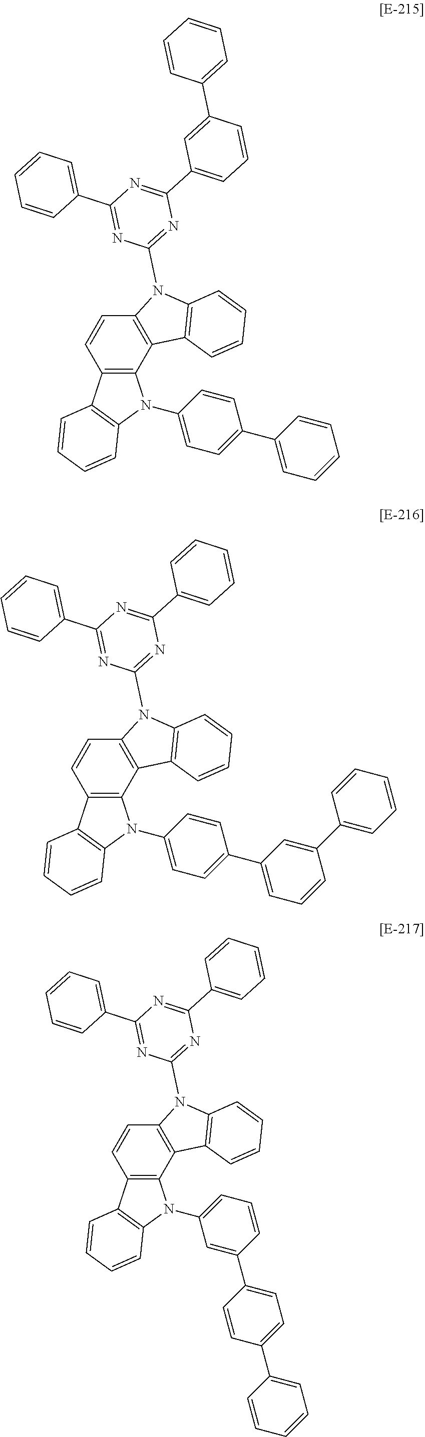 [SMILES: c1ccc(-c2ccc(-n3c4ccccc4c4ccc5c(c6ccccc6n5-c5nc(-c6ccccc6)nc(-c6cccc(-c7ccccc7)c6)n5)c43)cc2)cc1 ; c1ccc(-c2cccc(-c3ccc(-n4c5ccccc5c5ccc6c(c7ccccc7n6-c6nc(-c7ccccc7)nc(-c7ccccc7)n6)c54)cc3)c2)cc1 ; c1ccc(-c2ccc(-c3cccc(-n4c5ccccc5c5ccc6c(c7ccccc7n6-c6nc(-c7ccccc7)nc(-c7ccccc7)n6)c54)c3)cc2)cc1]

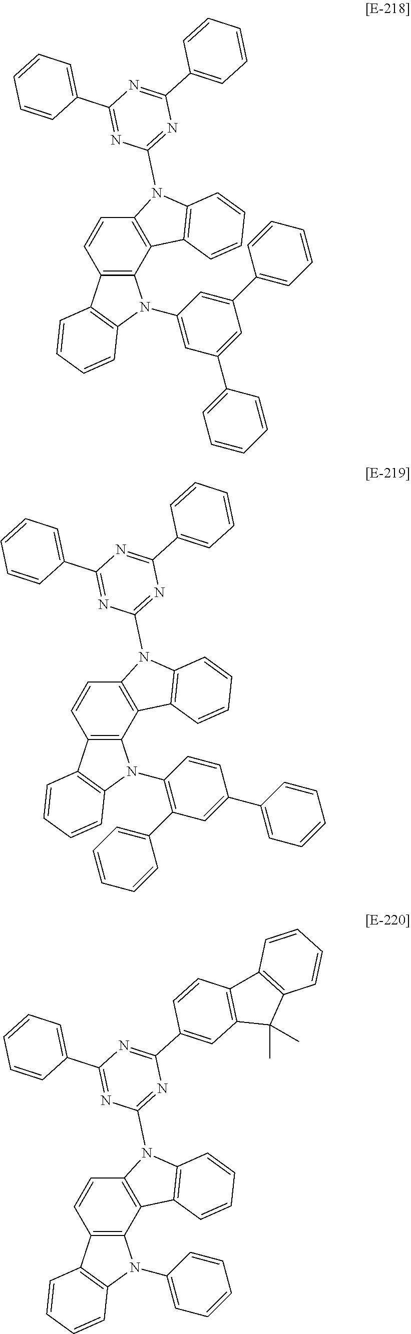

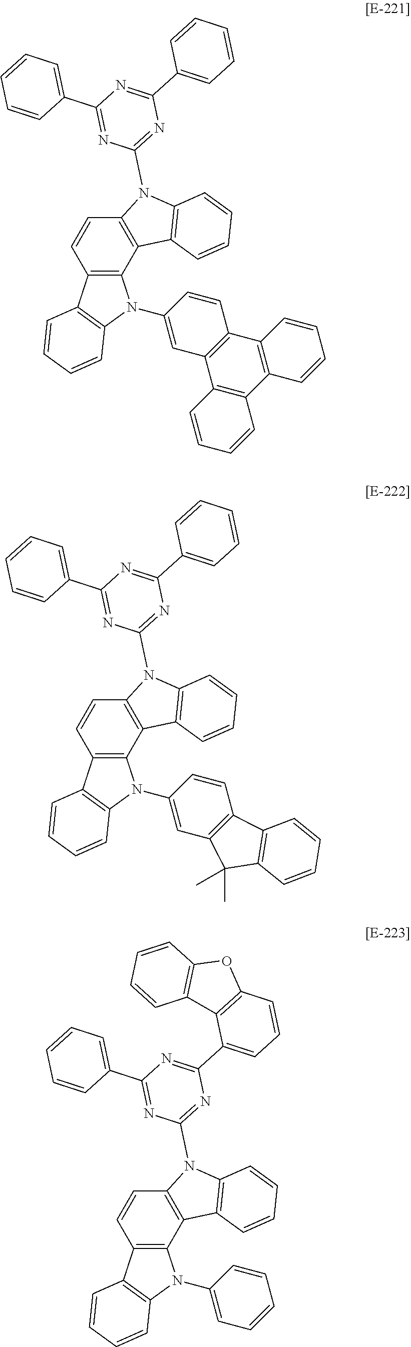 [SMILES: c1ccc(-c2nc(-c3ccccc3)nc(-n3c4ccccc4c4c3ccc3c5ccccc5n(-c5ccc6c7ccccc7c7ccccc7c6c5)c34)n2)cc1 ; CC1(C)c2ccccc2-c2ccc(-n3c4ccccc4c4ccc5c(c6ccccc6n5-c5nc(-c6ccccc6)nc(-c6ccccc6)n5)c43)cc21 ; c1ccc(-c2nc(-c3cccc4oc5ccccc5c34)nc(-n3c4ccccc4c4c3ccc3c5ccccc5n(-c5ccccc5)c34)n2)cc1]

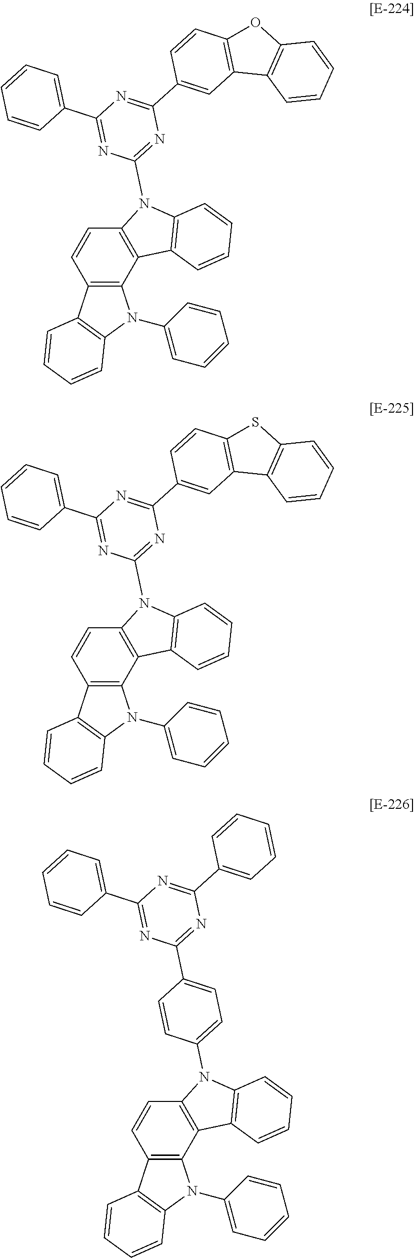 [SMILES: c1ccc(-c2nc(-c3ccc4oc5ccccc5c4c3)nc(-n3c4ccccc4c4c3ccc3c5ccccc5n(-c5ccccc5)c34)n2)cc1 ; c1ccc(-c2nc(-c3ccc4sc5ccccc5c4c3)nc(-n3c4ccccc4c4c3ccc3c5ccccc5n(-c5ccccc5)c34)n2)cc1 ; c1ccc(-c2nc(-c3ccccc3)nc(-c3ccc(-n4c5ccccc5c5c4ccc4c6ccccc6n(-c6ccccc6)c45)cc3)n2)cc1]

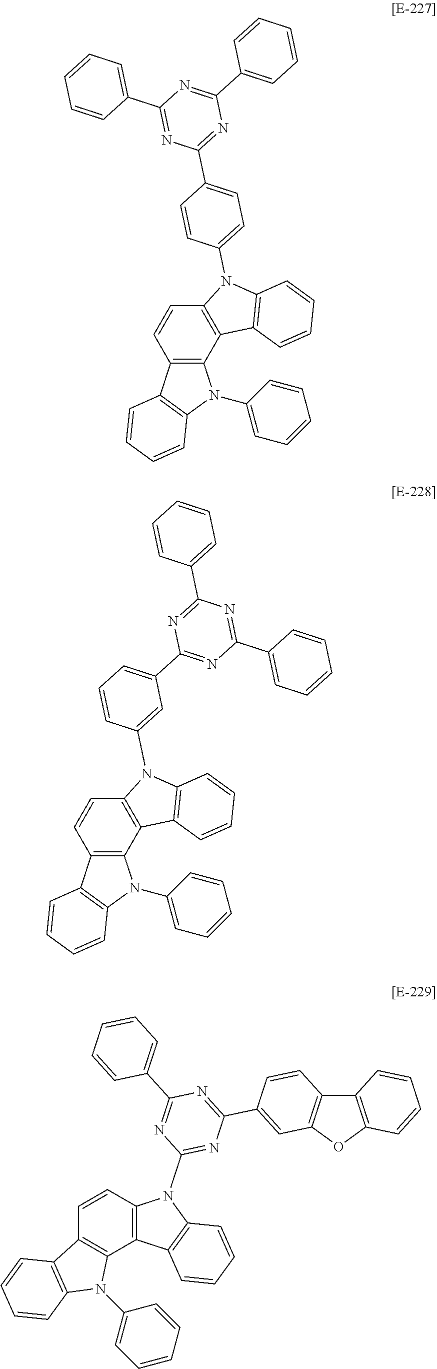

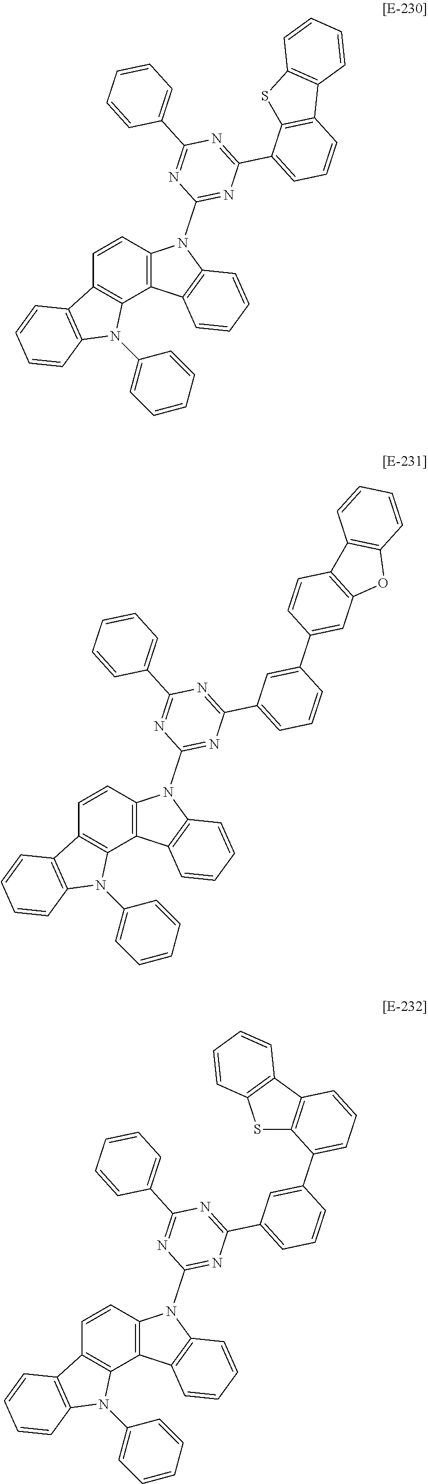 [SMILES: c1ccc(-c2nc(-c3cccc4c3sc3ccccc34)nc(-n3c4ccccc4c4c3ccc3c5ccccc5n(-c5ccccc5)c34)n2)cc1 ; c1ccc(-c2nc(-c3cccc(-c4ccc5c(c4)oc4ccccc45)c3)nc(-n3c4ccccc4c4c3ccc3c5ccccc5n(-c5ccccc5)c34)n2)cc1 ; c1ccc(-c2nc(-c3cccc(-c4cccc5c4sc4ccccc45)c3)nc(-n3c4ccccc4c4c3ccc3c5ccccc5n(-c5ccccc5)c34)n2)cc1]

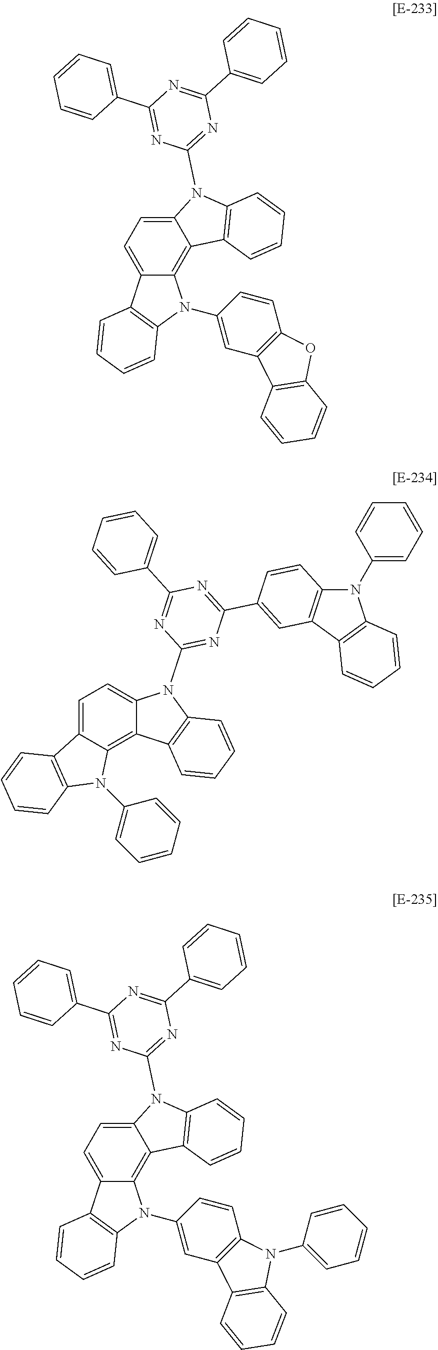 [SMILES: c1ccc(-c2nc(-c3ccccc3)nc(-n3c4ccccc4c4c3ccc3c5ccccc5n(-c5ccc6oc7ccccc7c6c5)c34)n2)cc1 ; c1ccc(-c2nc(-c3ccc4c(c3)c3ccccc3n4-c3ccccc3)nc(-n3c4ccccc4c4c3ccc3c5ccccc5n(-c5ccccc5)c34)n2)cc1 ; c1ccc(-c2nc(-c3ccccc3)nc(-n3c4ccccc4c4c3ccc3c5ccccc5n(-c5ccc6c(c5)c5ccccc5n6-c5ccccc5)c34)n2)cc1]

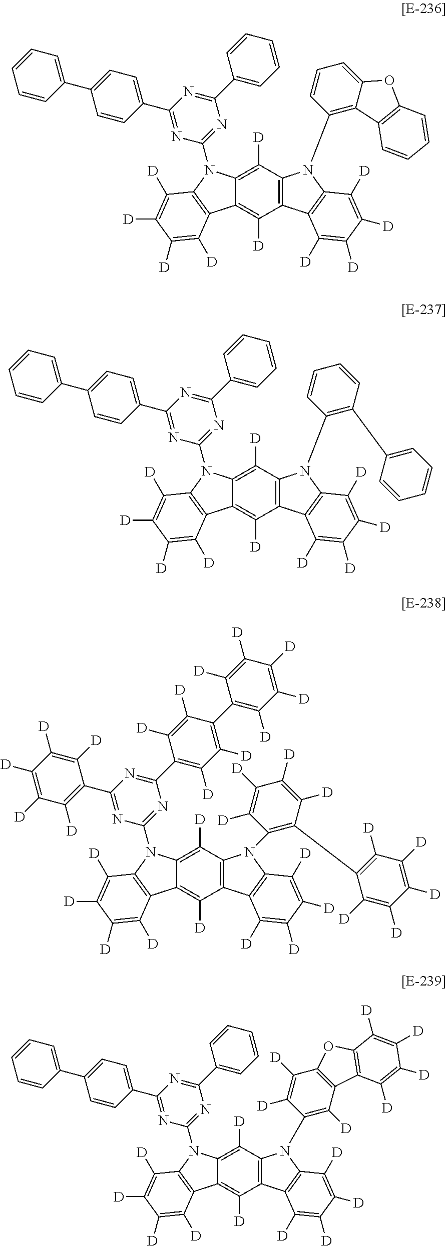

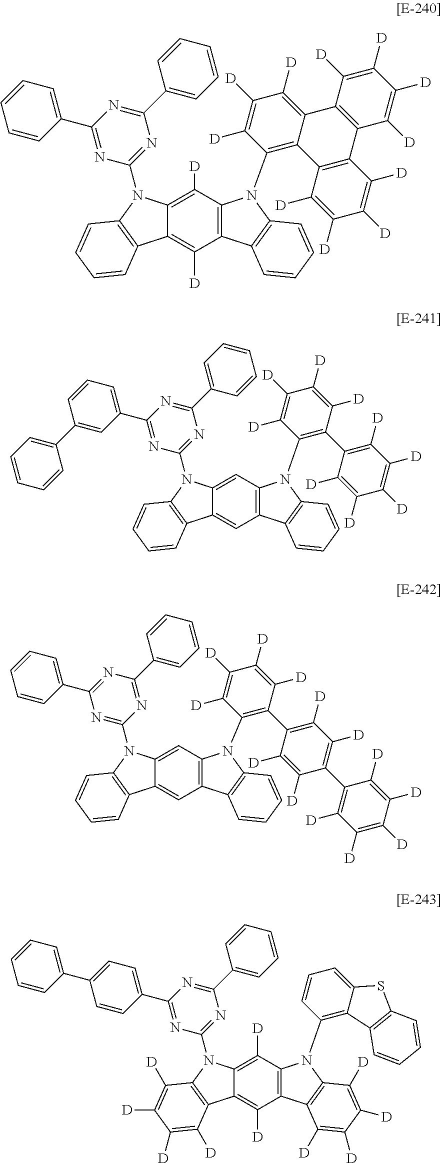

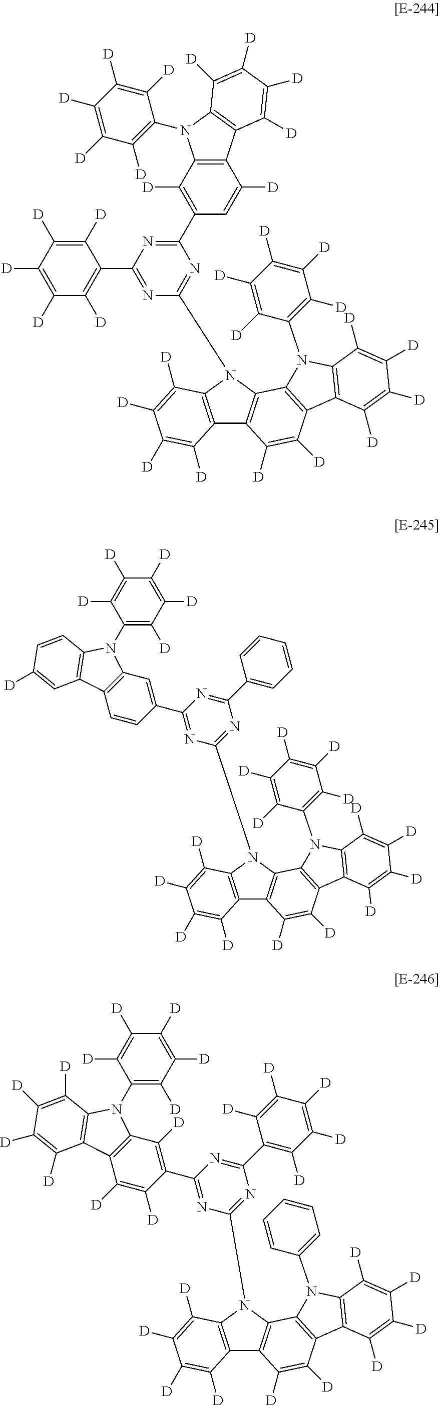

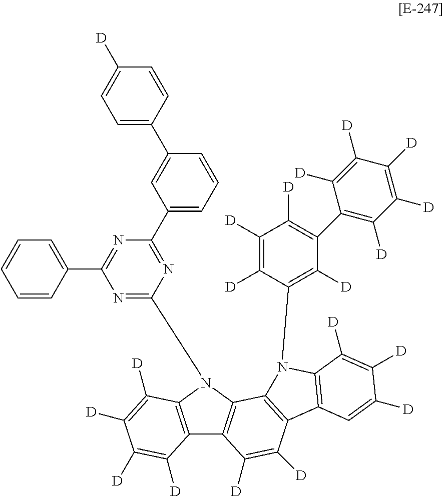 [SMILES: [2H]c1ccc(-c2cccc(-c3nc(-c4ccccc4)nc(-n4c5c([2H])c([2H])c([2H])c([2H])c5c5c([2H])c([2H])c6c7cc([2H])c([2H])c([2H])c7n(-c7c([2H])c([2H])c([2H])c(-c8c([2H])c([2H])c([2H])c([2H])c8[2H])c7[2H])c6c54)n3)c2)cc1]

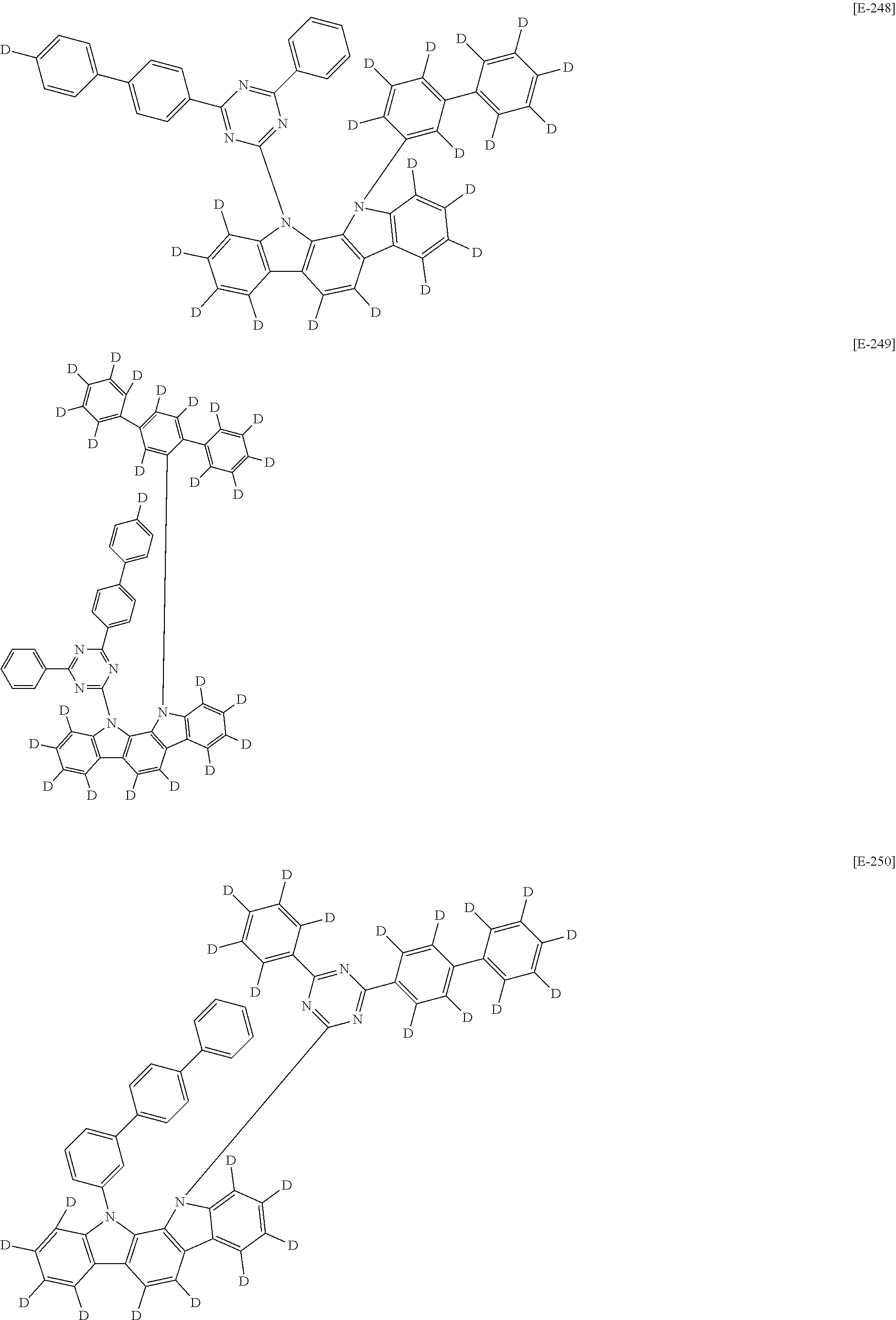

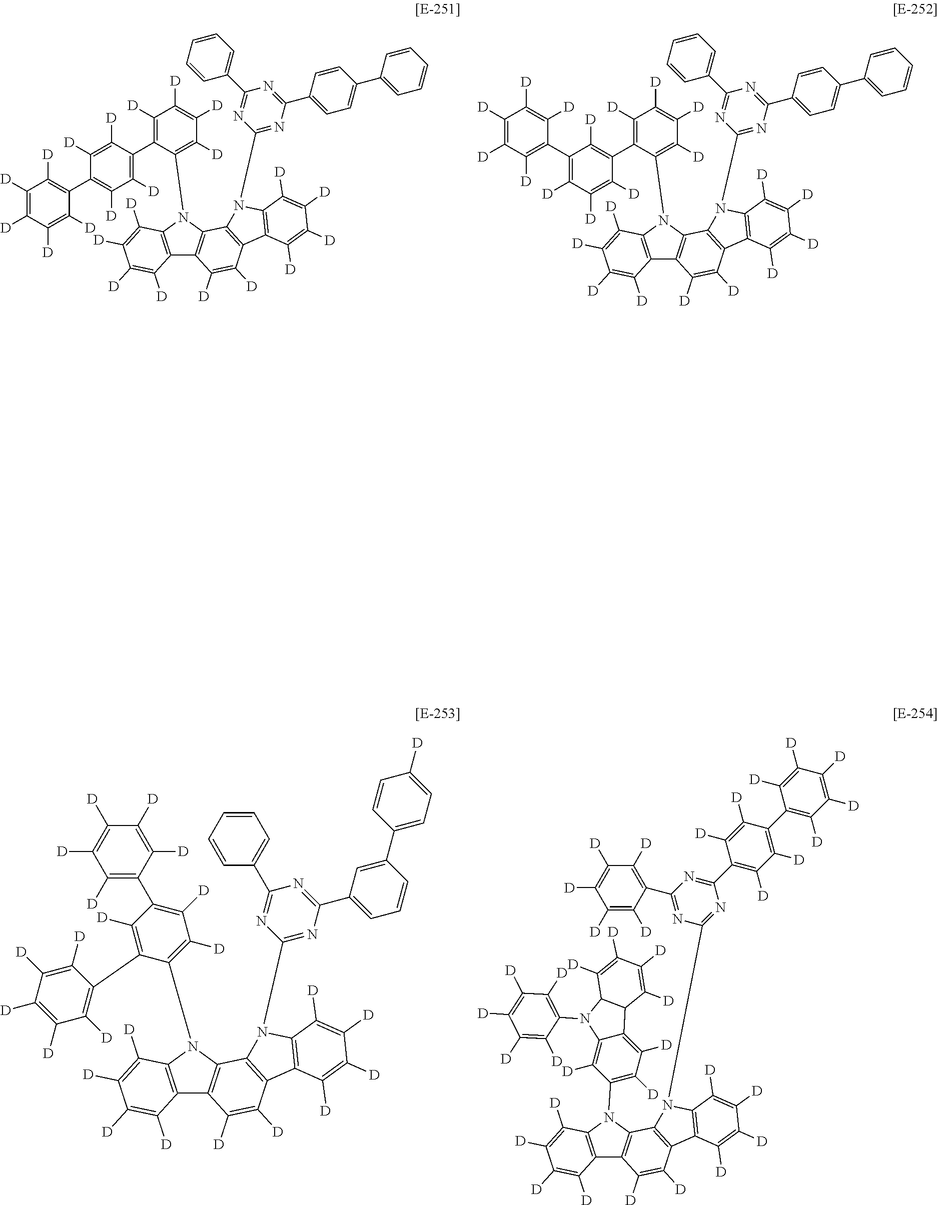

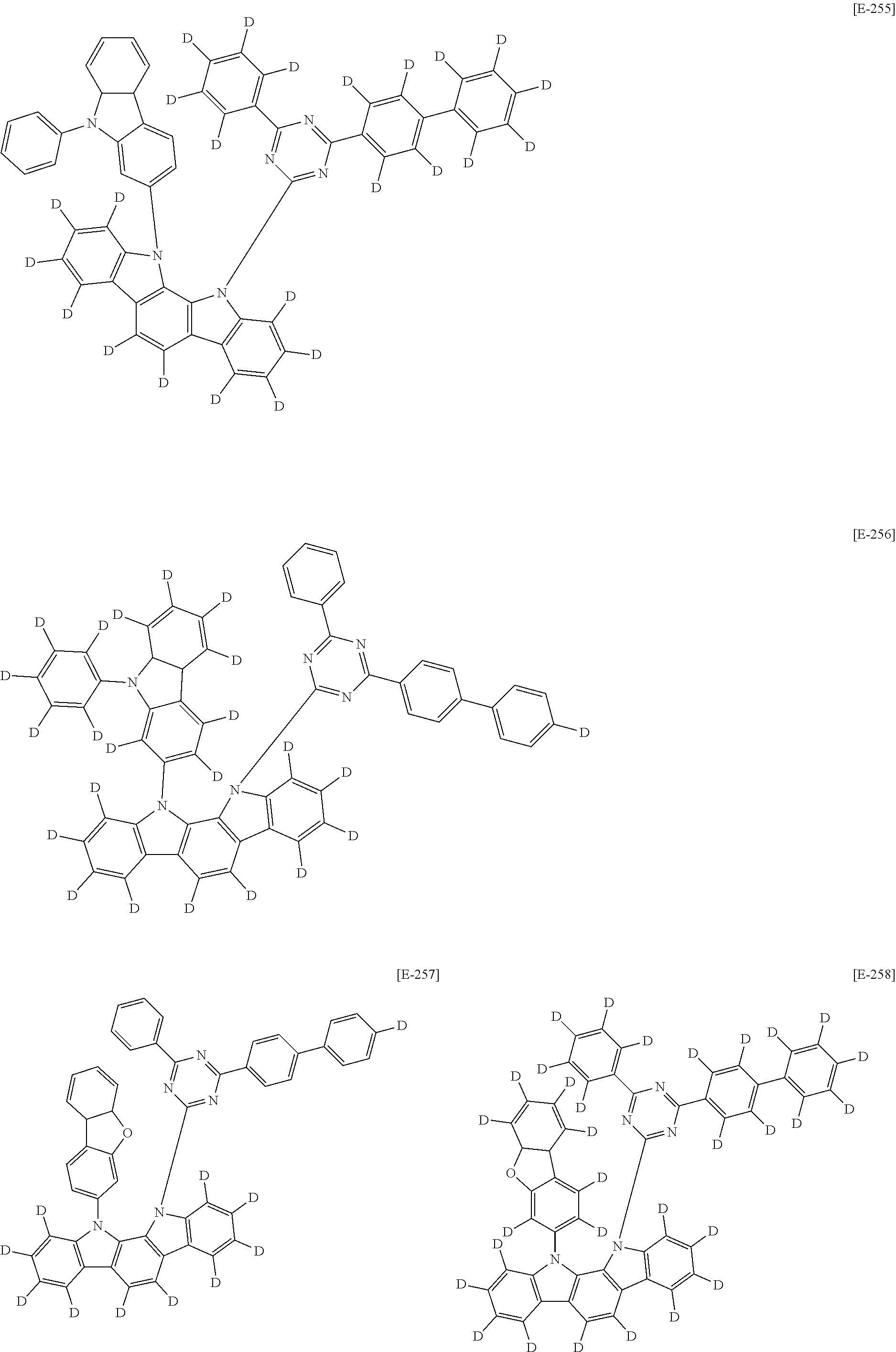

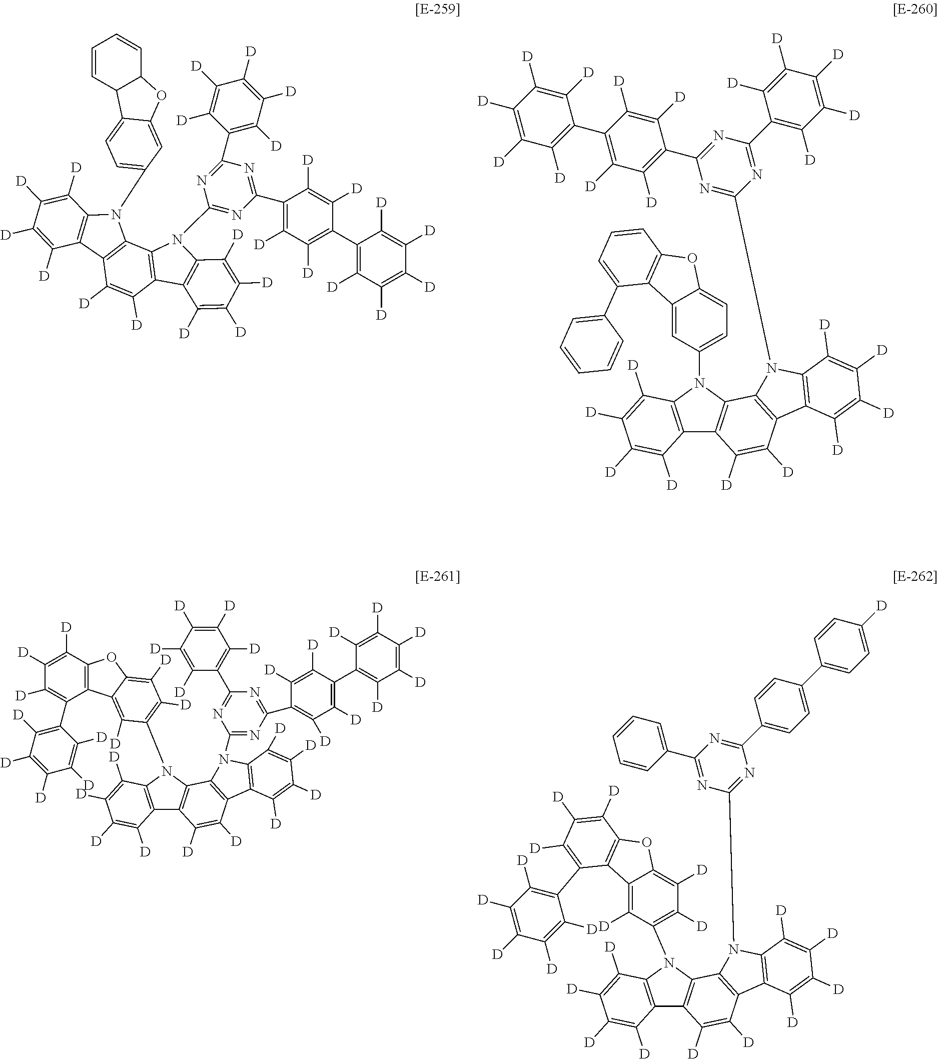

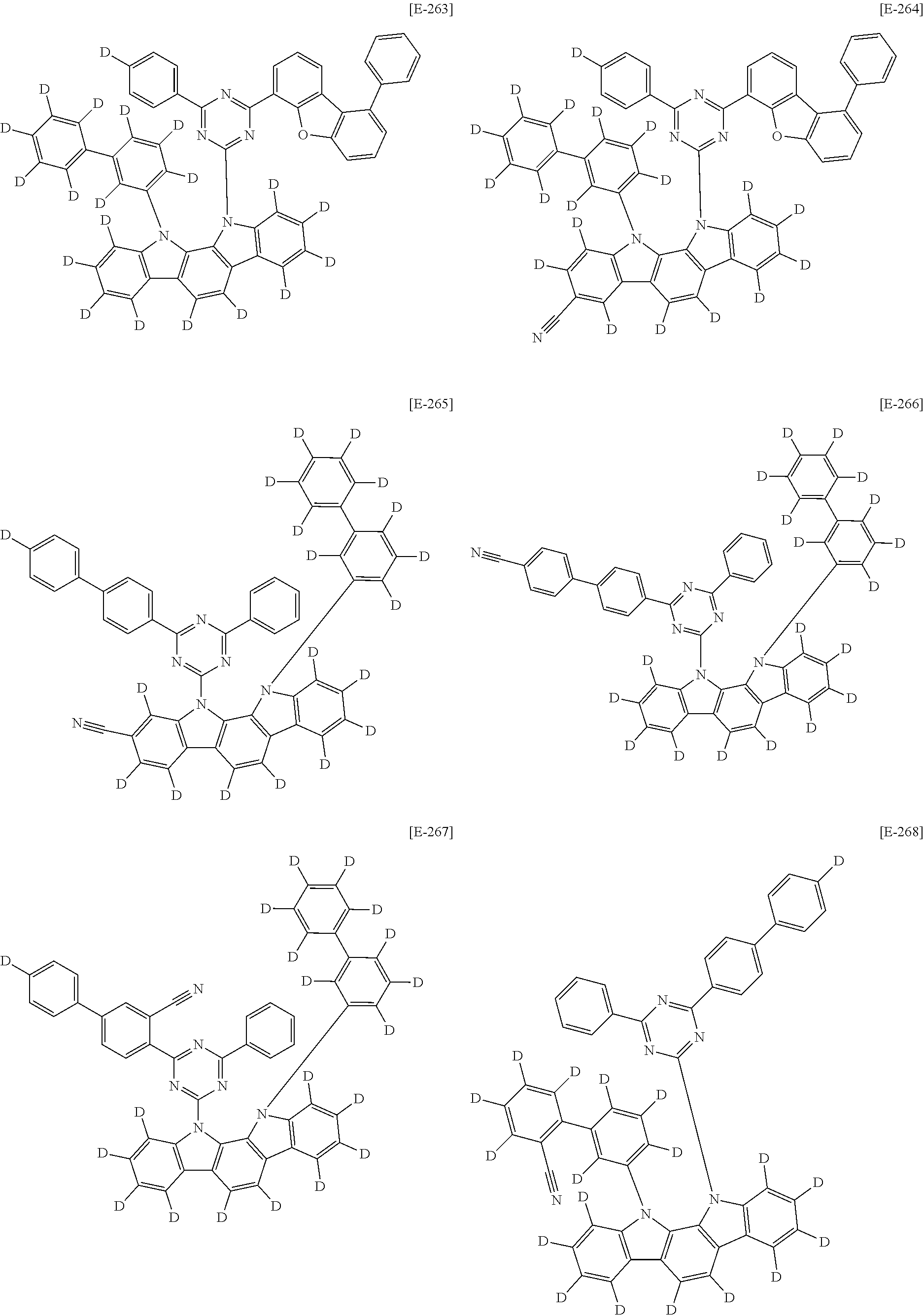

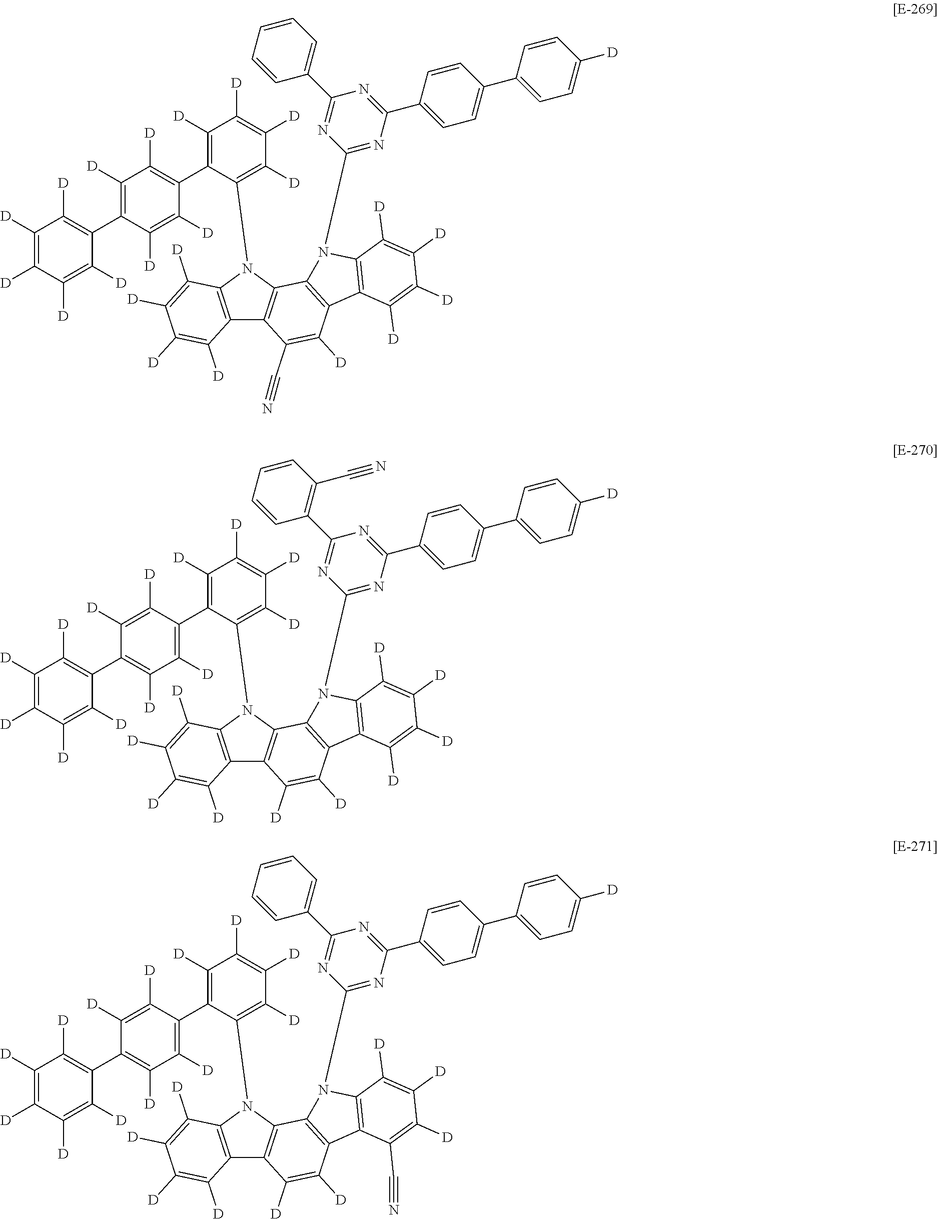

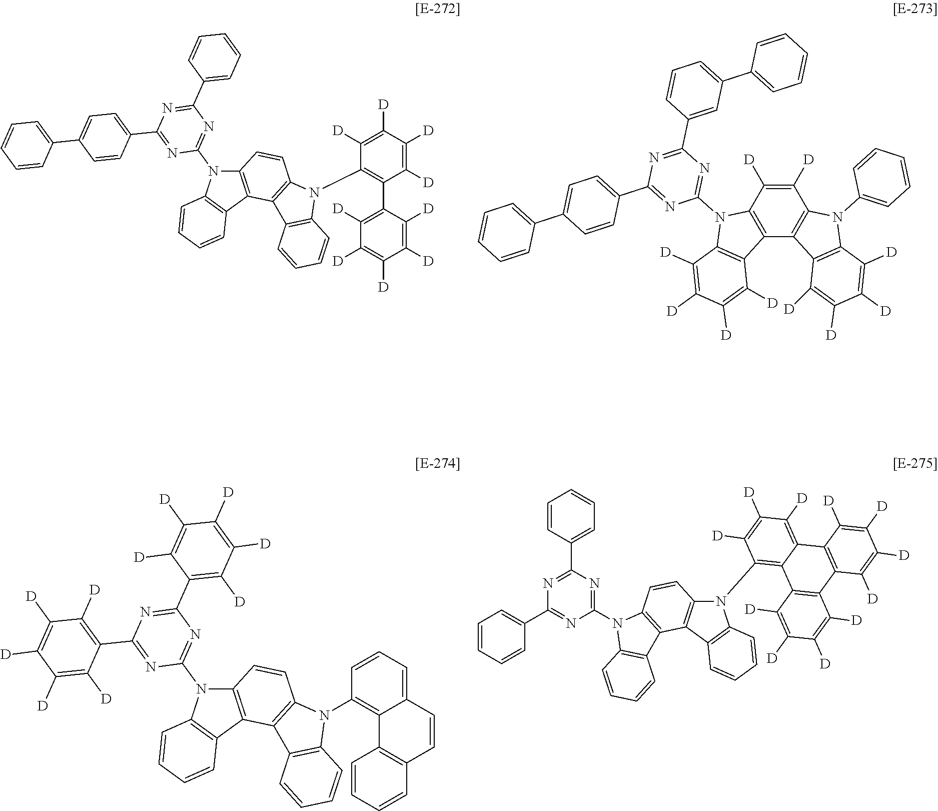

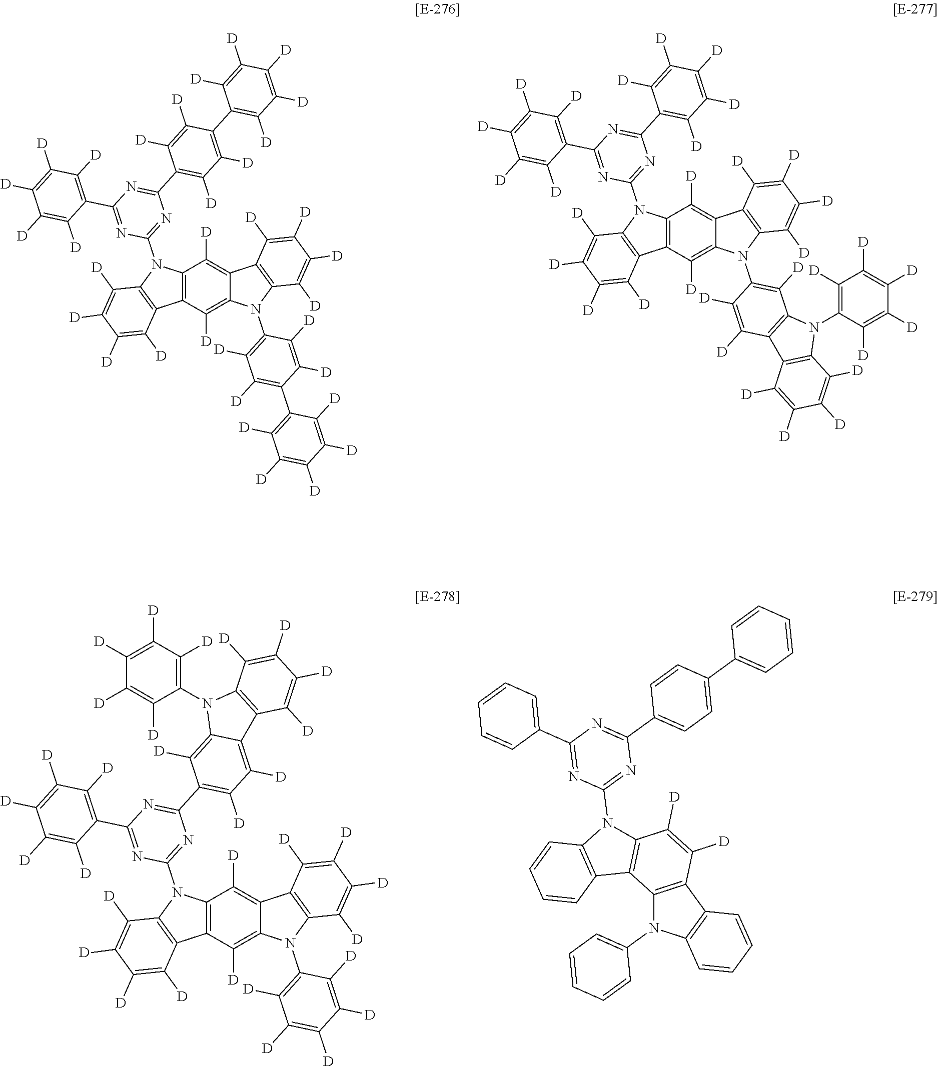

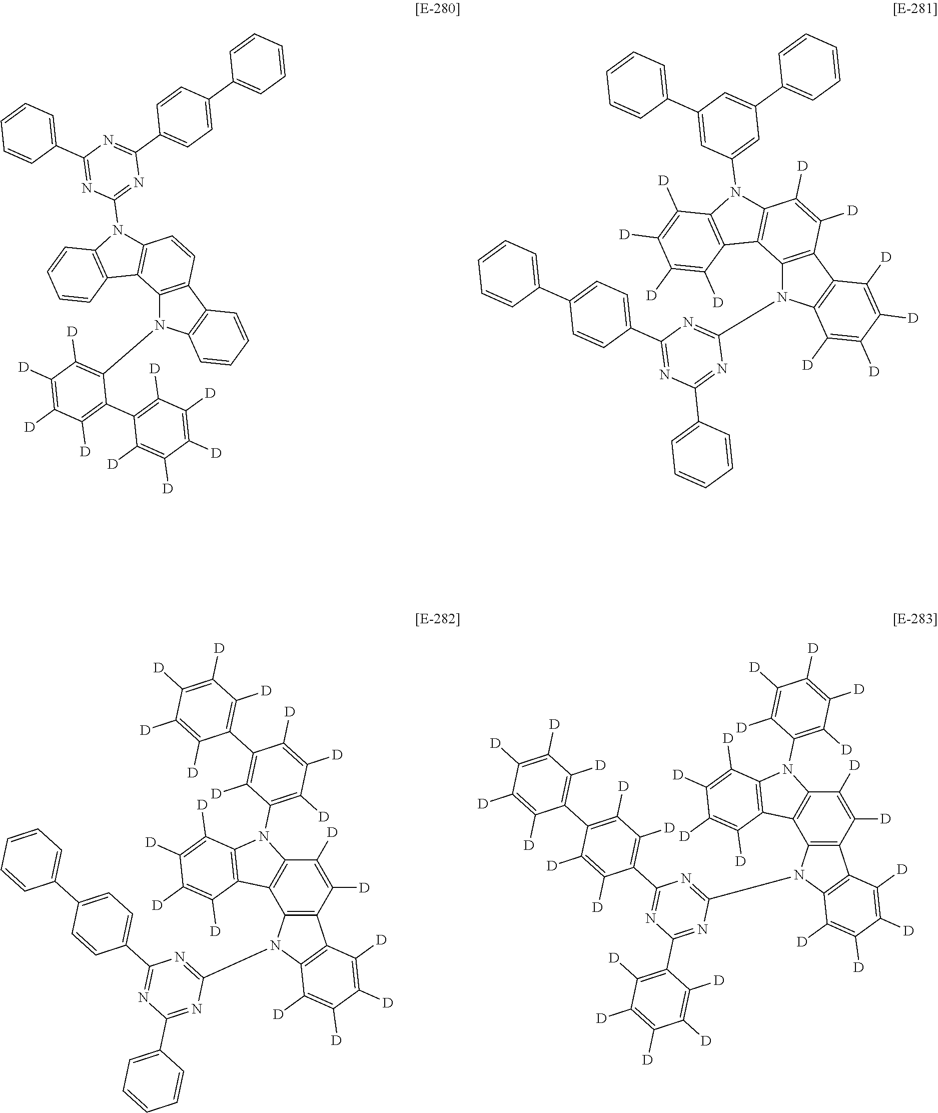

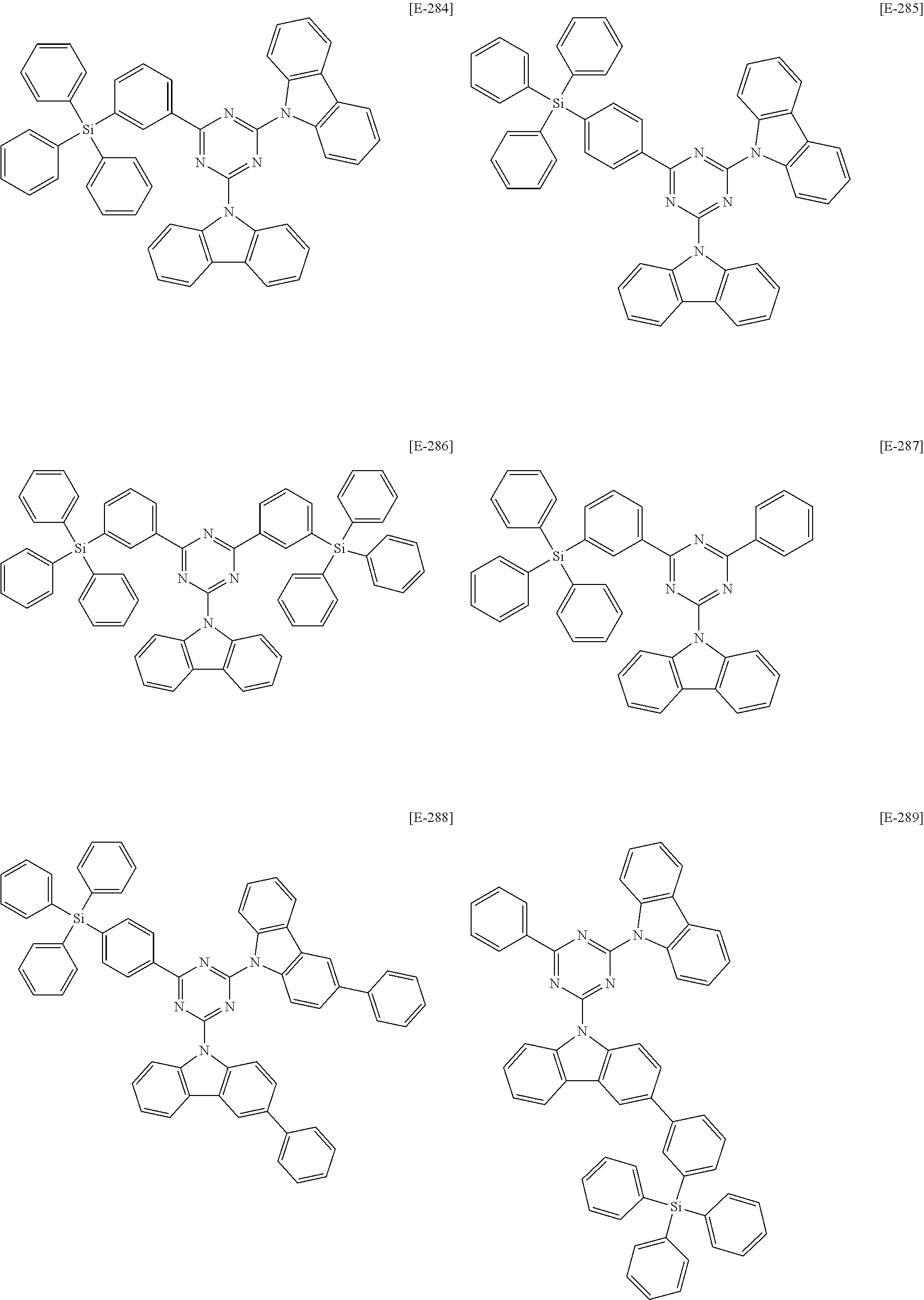

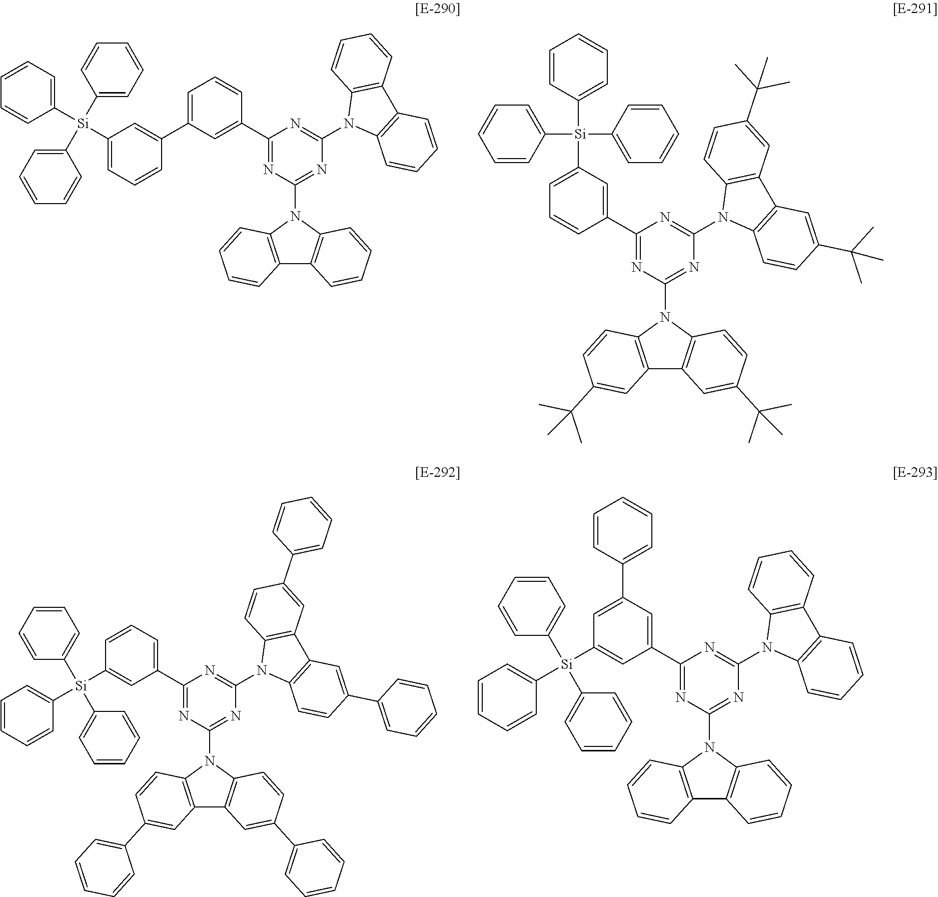

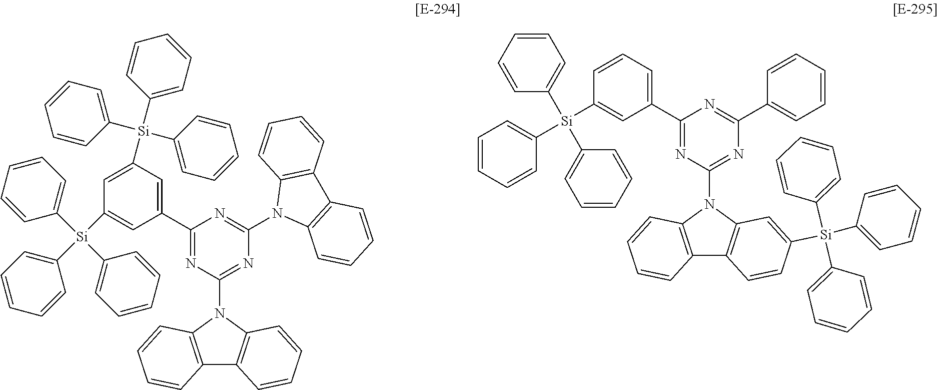 [SMILES: c1ccc([Si](c2ccccc2)(c2ccccc2)c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1 ; c1ccc(-c2nc(-c3cccc([Si](c4ccccc4)(c4ccccc4)c4ccccc4)c3)nc(-n3c4ccccc4c4ccc([Si](c5ccccc5)(c5ccccc5)c5ccccc5)cc43)n2)cc1]

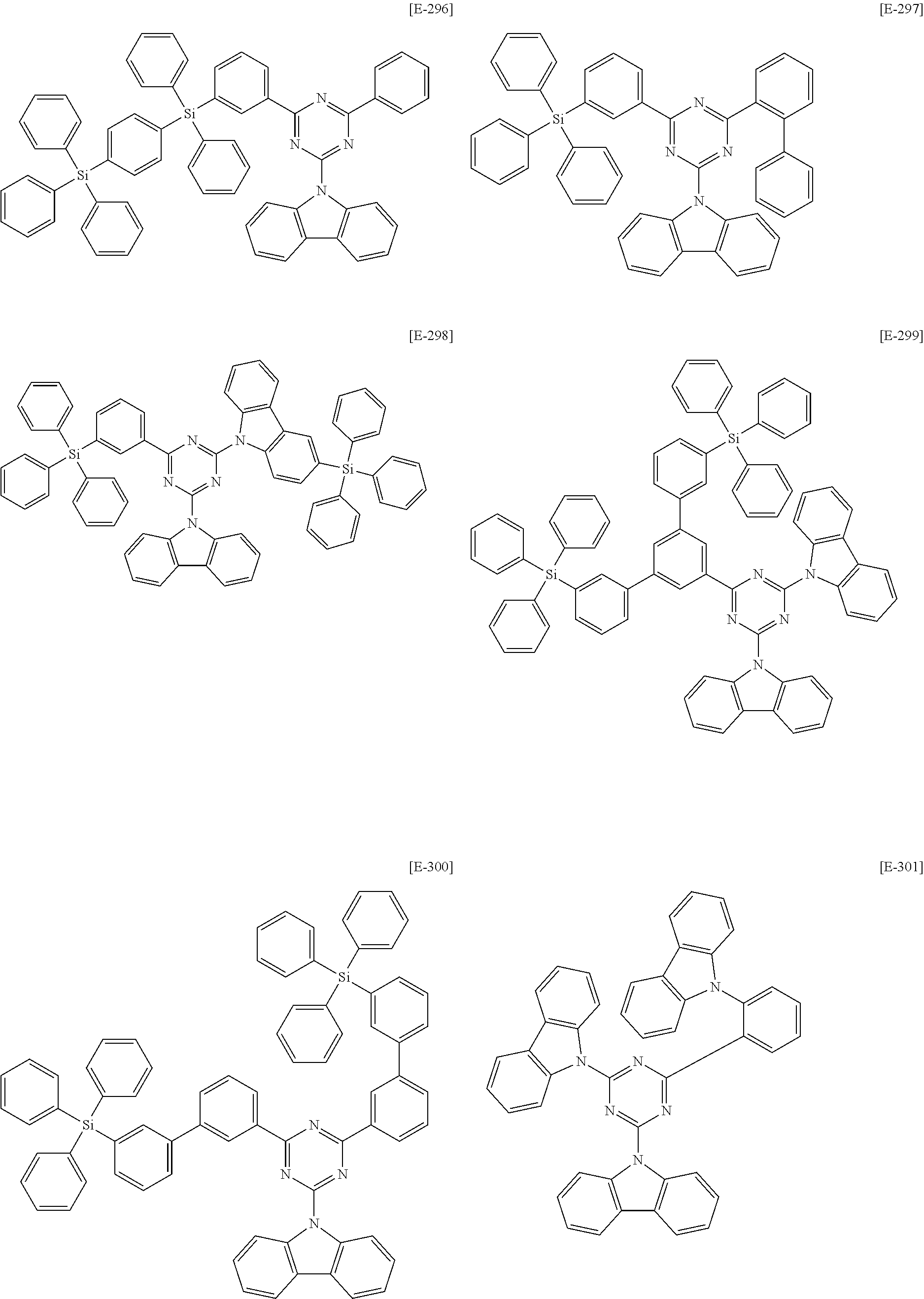

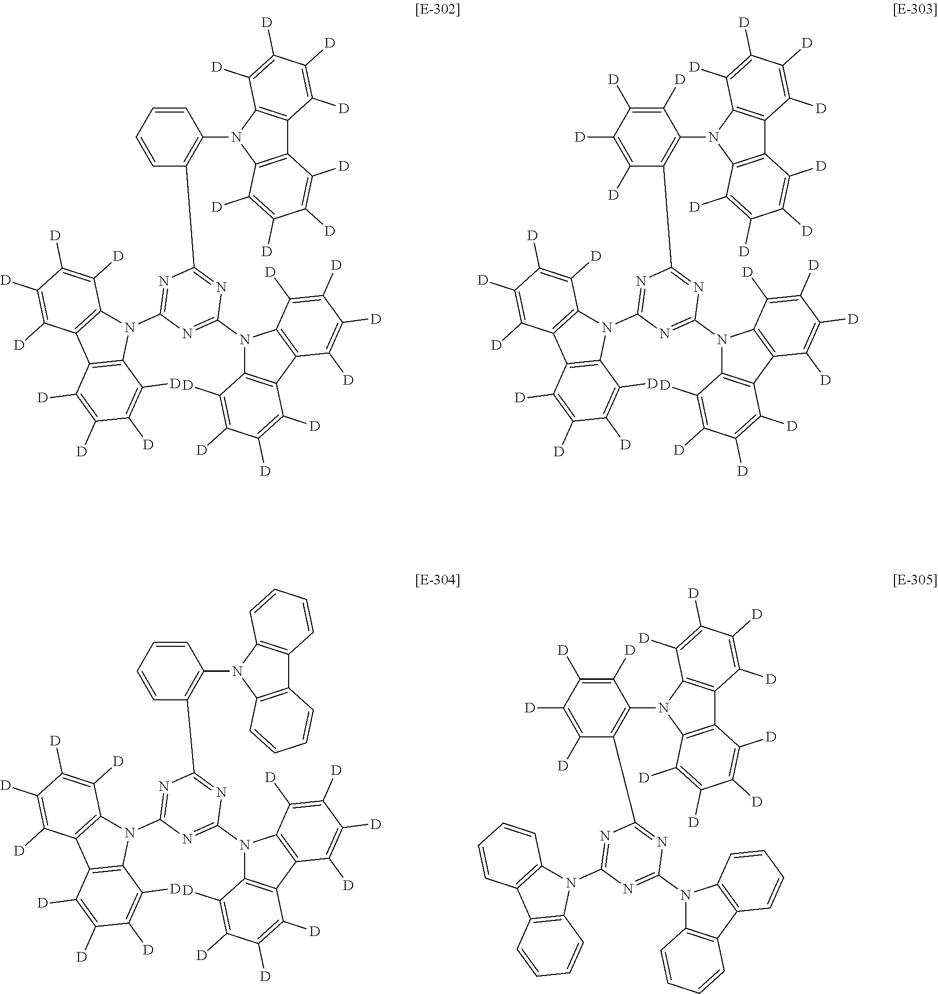

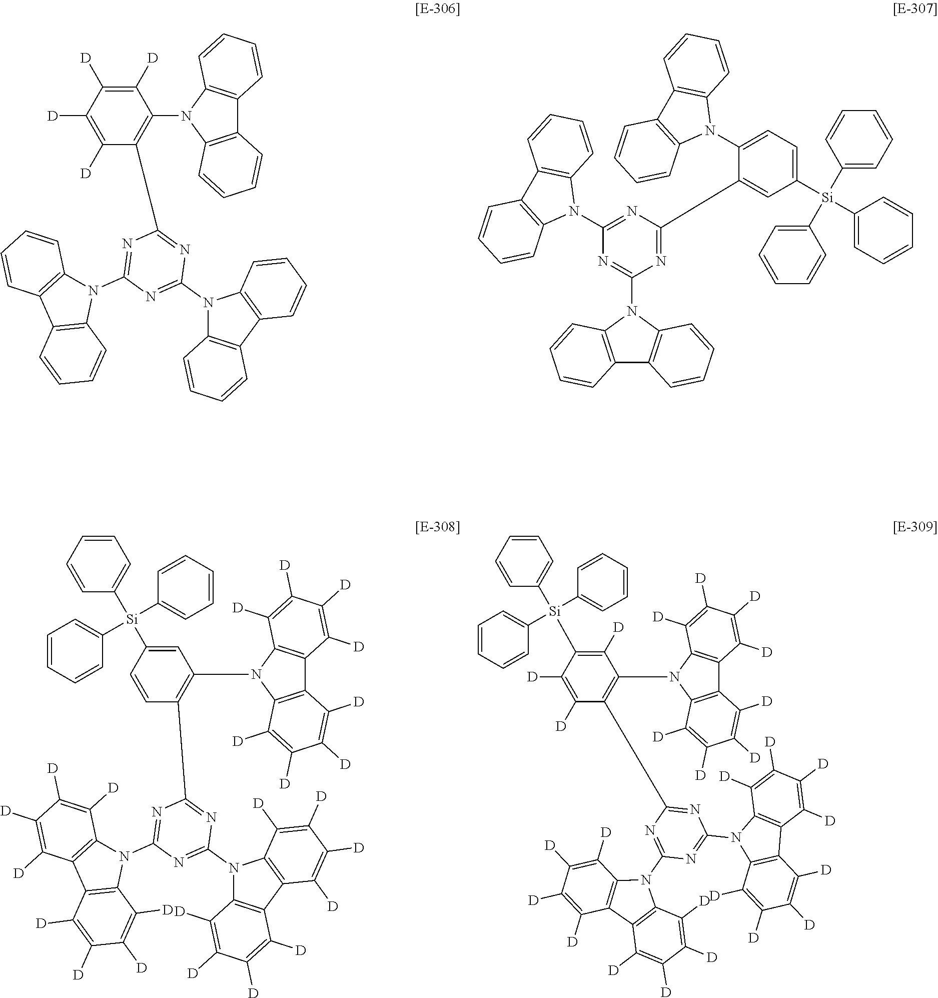

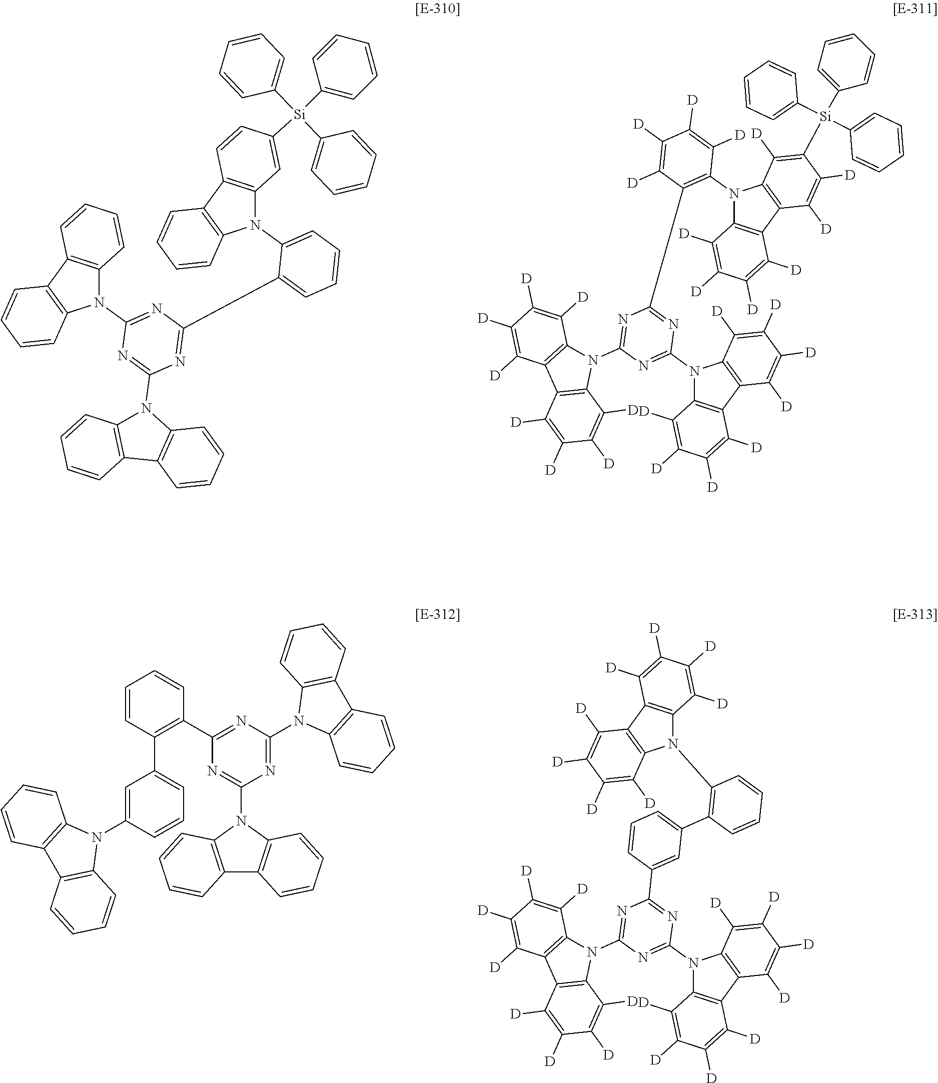

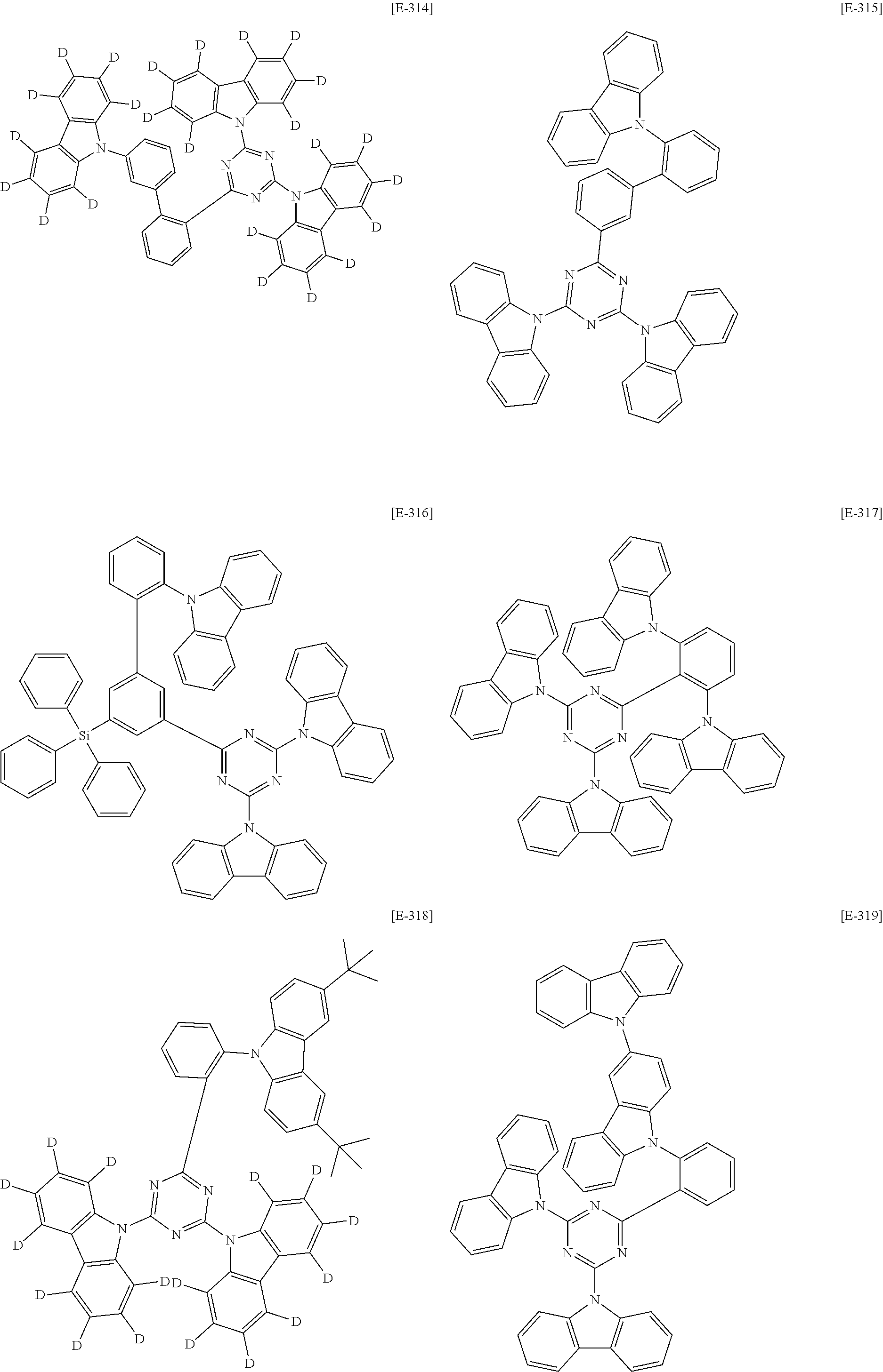

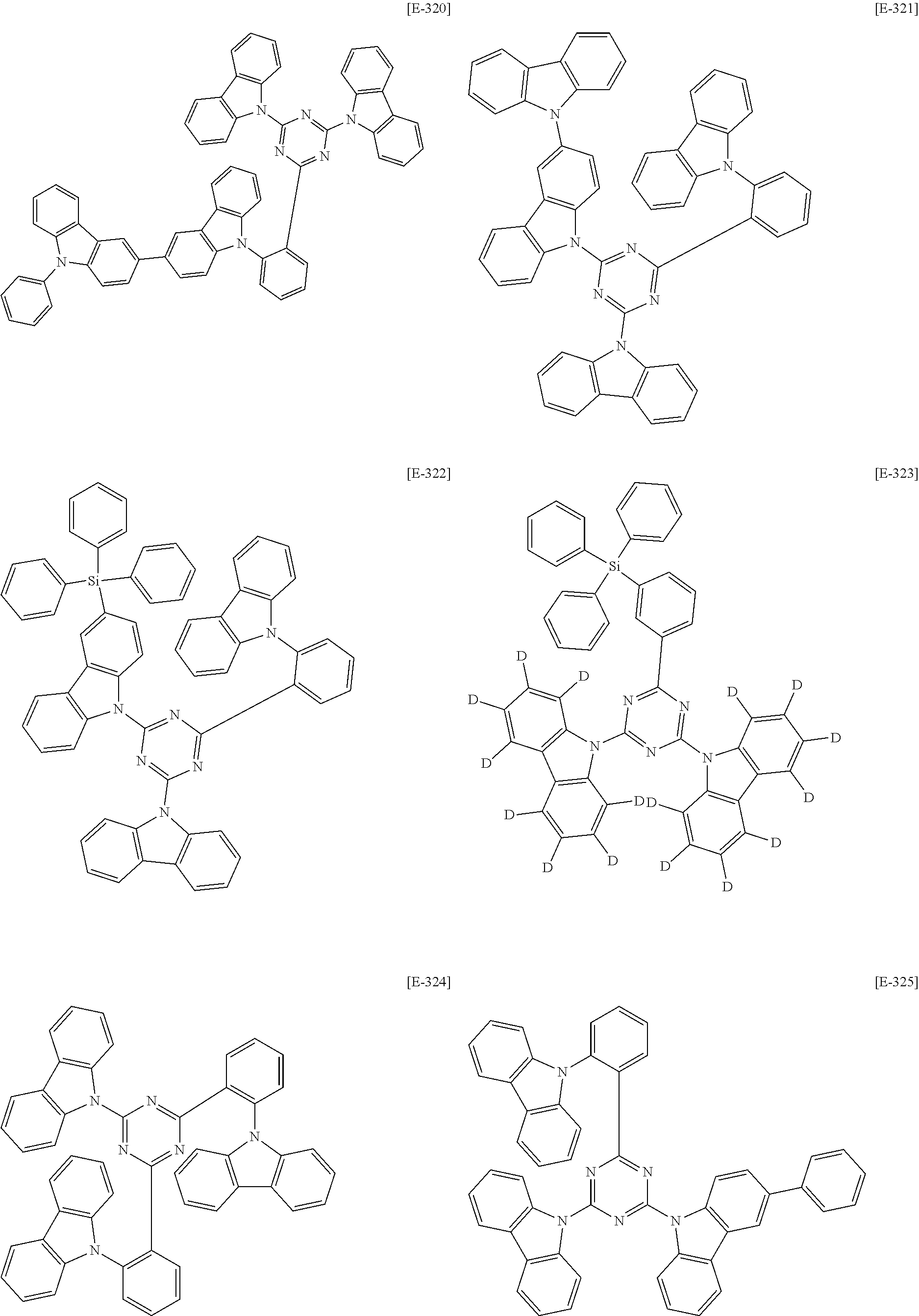

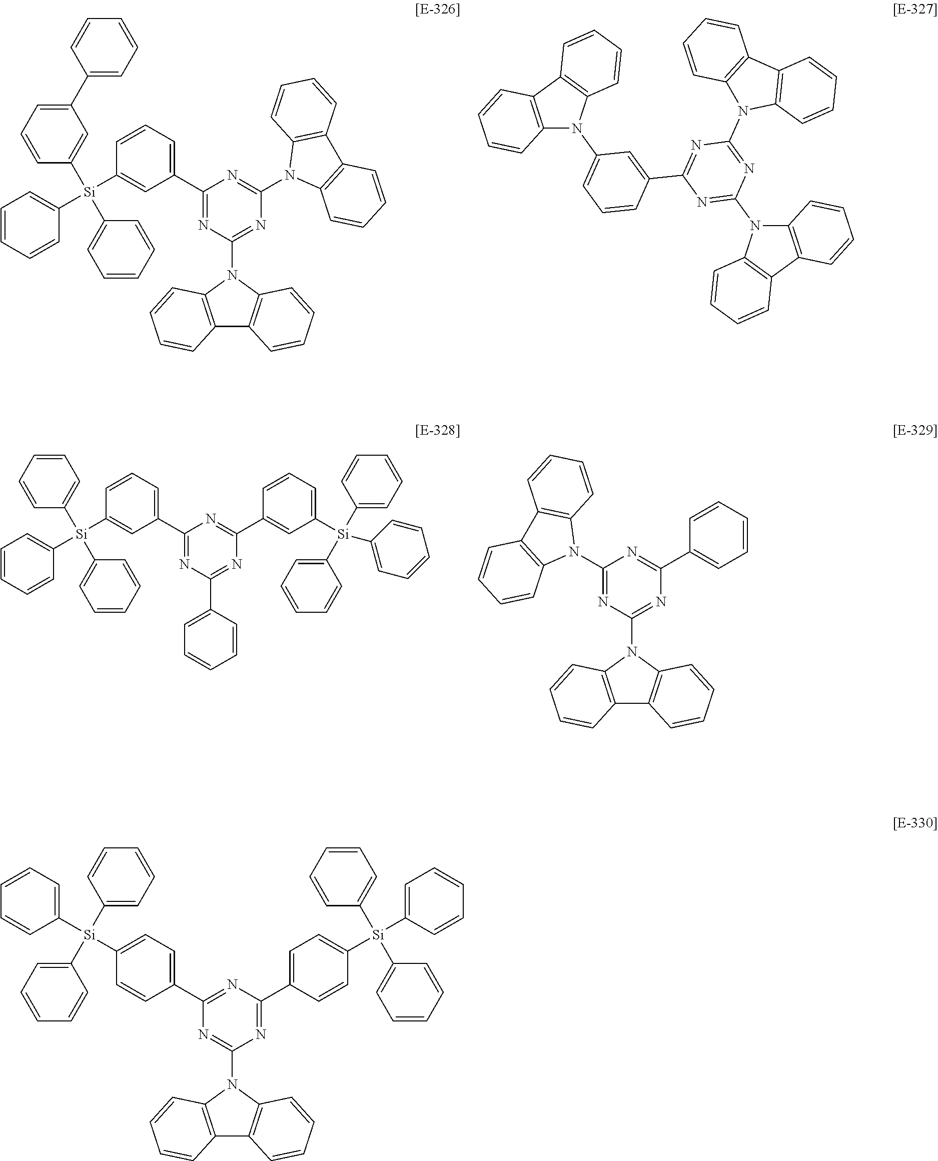 [SMILES: c1ccc(-c2cccc([Si](c3ccccc3)(c3ccccc3)c3cccc(-c4nc(-n5c6ccccc6c6ccccc65)nc(-n5c6ccccc6c6ccccc65)n4)c3)c2)cc1 ; c1cc(-c2nc(-n3c4ccccc4c4ccccc43)nc(-n3c4ccccc4c4ccccc43)n2)cc(-n2c3ccccc3c3ccccc32)c1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2nc(-n3c4ccccc4c4ccccc43)nc(-n3c4ccccc4c4ccccc43)n2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1]

The composition for an organic optoelectronic device may include the first compound and the second compound in various ratios.

For example, the composition for an organic optoelectronic device may include the first compound and the second compound in a weight ratio of about 10:90 to about 90:10, e.g., in a weight ratio of about 20:80 to about 80:20, about 30:70 to about 70:30, about 40:60 to about 60:40, or about 50:50.

For example, the first compound may be included in an amount that is equal to or greater than the second compound. For example, the first compound may be included in about 50 wt % to about 90 wt %, based on a total amount of the first compound and the second compound.

In another example, the first compound may be included in an amount that is less than or the same as the second compound. For example, the first compound may be included in about 10 wt % to about 50 wt %, based on a total amount of the first compound and the second compound.

The composition for an organic optoelectronic device may further include a light-emitting dopant in addition to the first compound and the second compound. The light-emitting dopant is a material mixed with the composition for an organic optoelectronic device in a small amount to cause light emission, and may be generally a material such as a metal complex that emits light by multiple excitation into a triplet or more. The light-emitting dopant may be, e.g., an inorganic, organic, or organic/inorganic compound, and may be included in one or two or more types. The light-emitting dopant may be, e.g., a phosphorescent sensitizer, a fluorescent dopant, or a combination thereof.

The phosphorescent sensitizer may be an organometallic compound and may effectively transfer energy received from the host to the fluorescent dopant. The phosphorescent sensitizer may increase energy transfer to the fluorescent dopant, causing excitons formed in the light emitting layer to emit light quickly inside the light emitting layer, thereby reducing deterioration of the light emitting diode.

The phosphorescent sensitizer may be, e.g., an organo-metal compound including iridium (Ir), platinum (Pt), osmium (Os), titanium (Ti), zirconium (Zr), hafnium (Hf), europium (Eu), terbium (Tb), thulium (Tm), rhodium (Rh), or a combination thereof, and may be, e.g., an organo-metal compound including an organic ligand including a nitrogen-containing ring. The nitrogen-containing ring may be, e.g., a substituted or unsubstituted pyridine, a substituted or unsubstituted pyrimidine, a substituted or unsubstituted triazine, a substituted or unsubstituted carbazole, a substituted or unsubstituted imidazole, a substituted or unsubstituted benzoimidazole, or a combination thereof. The phosphorescent sensitizer may be, e.g., any one of Compounds P1 to P52, below.

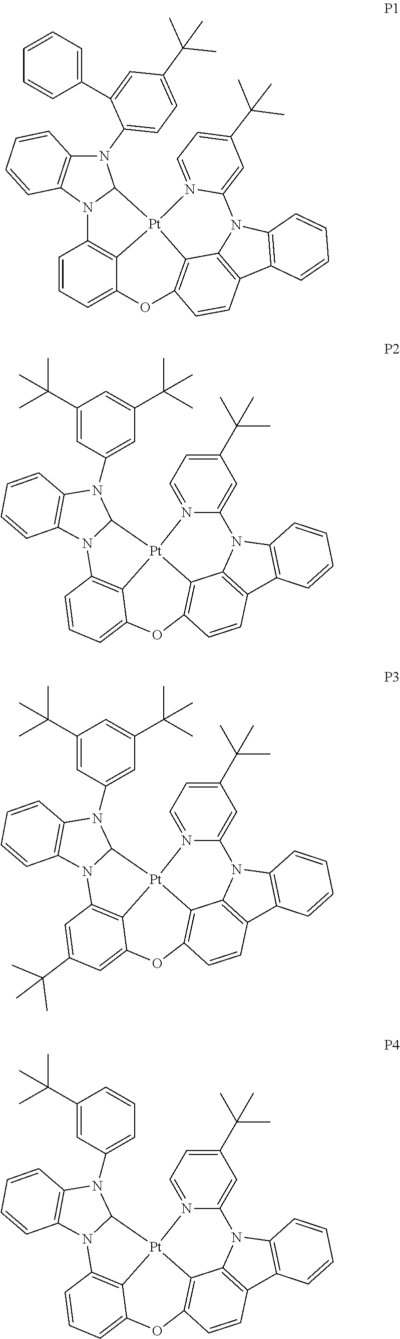

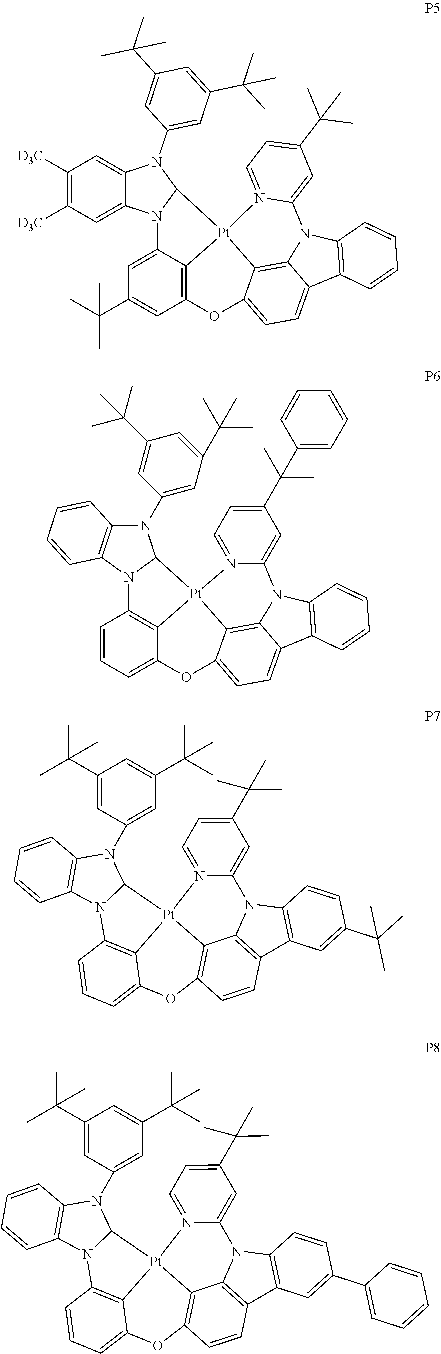

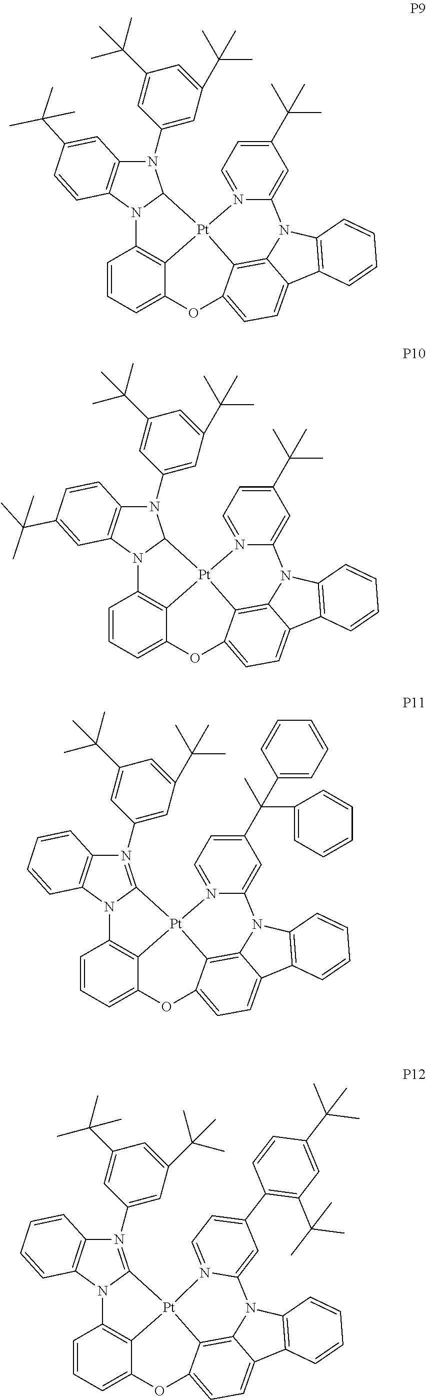

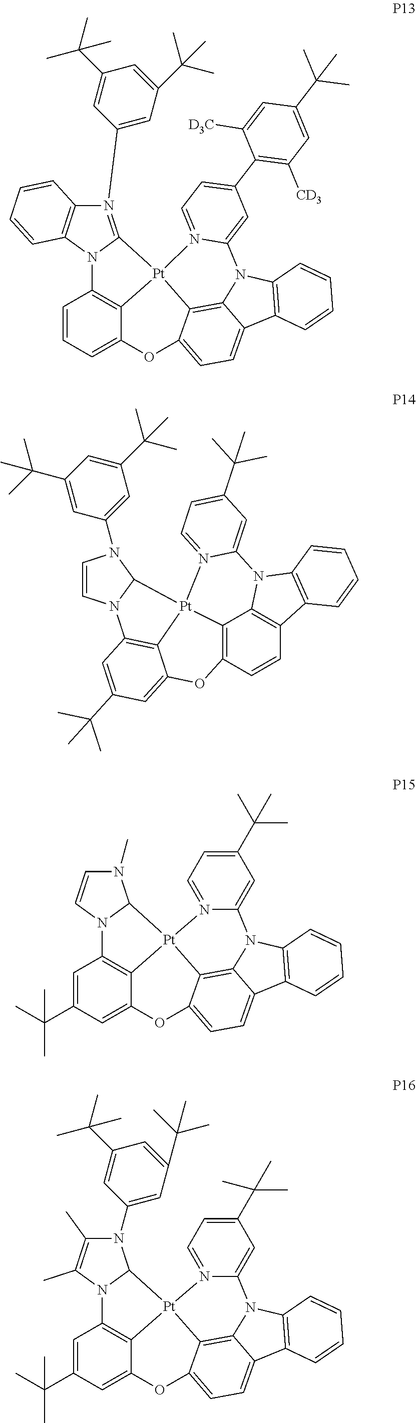

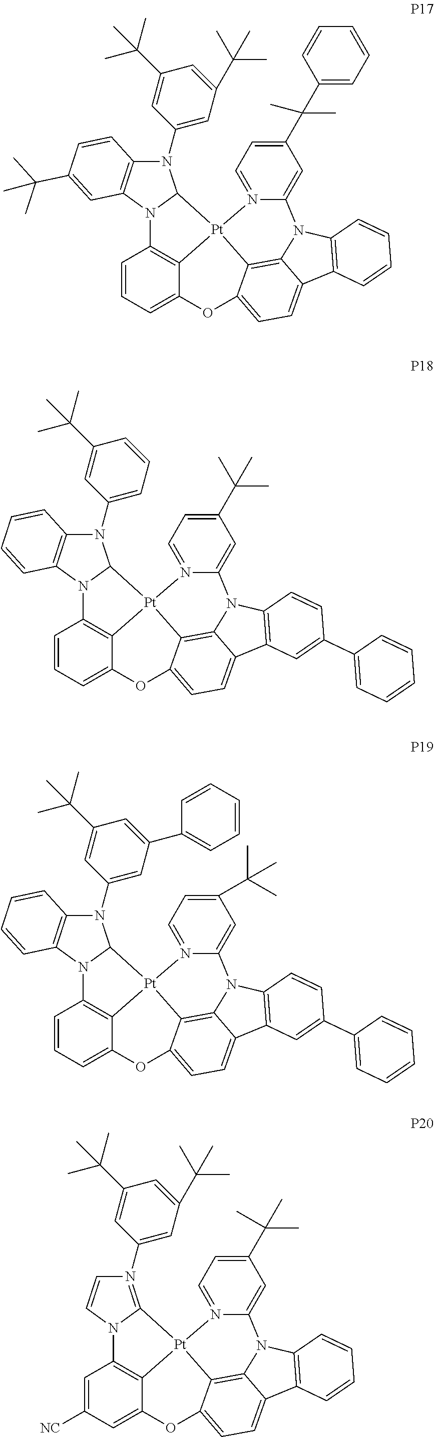

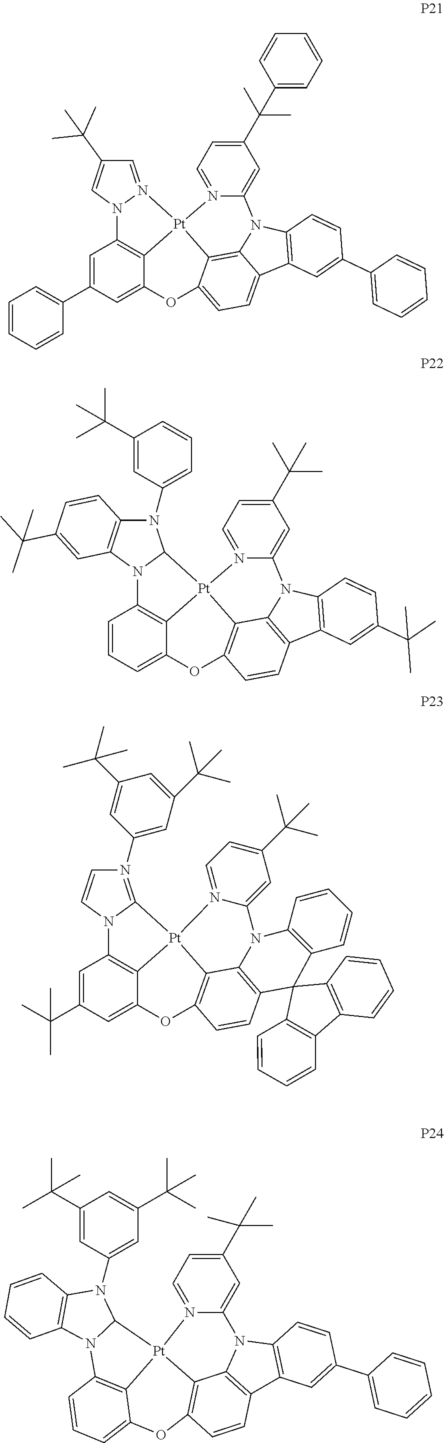

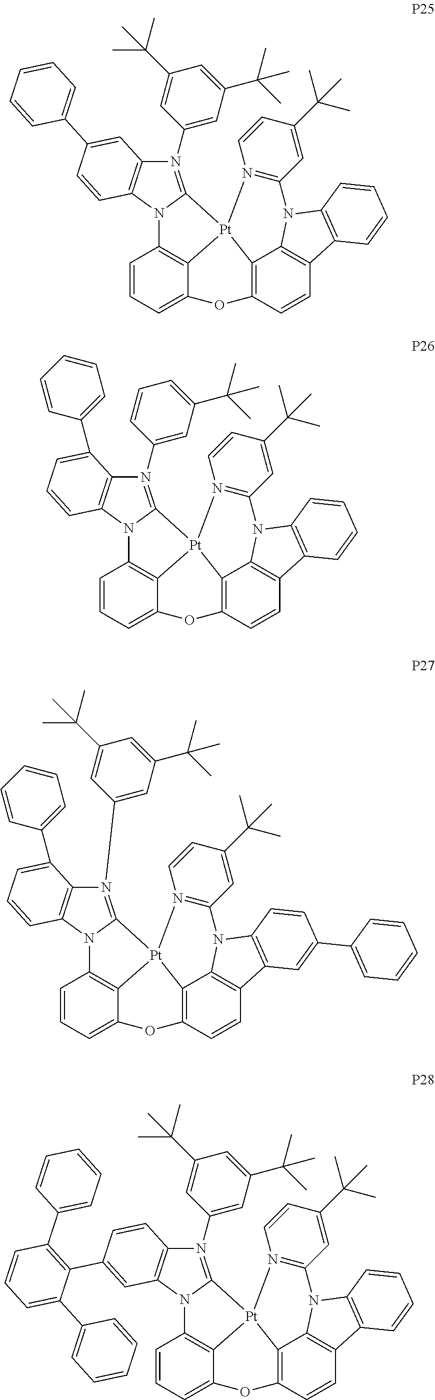

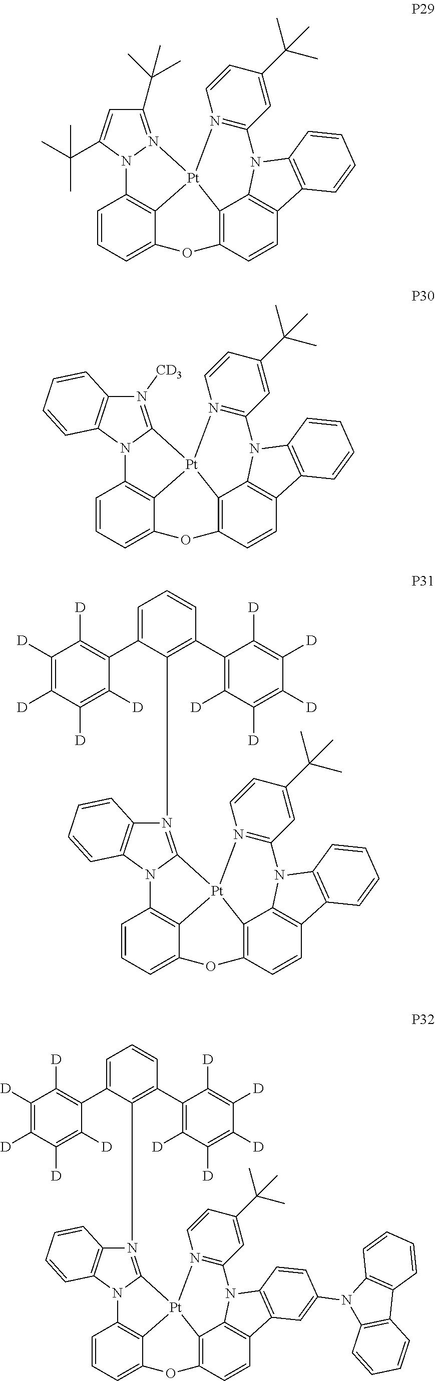

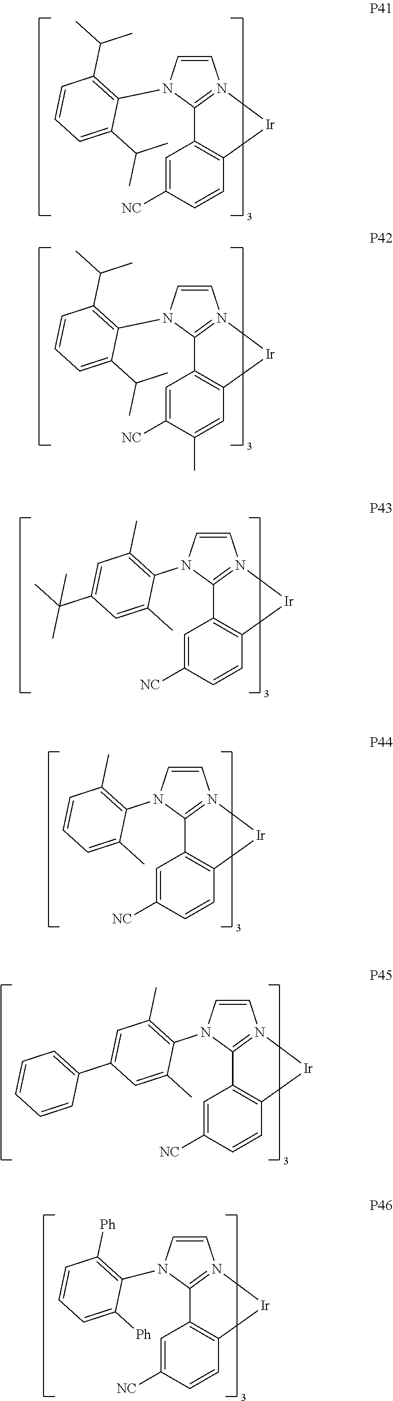

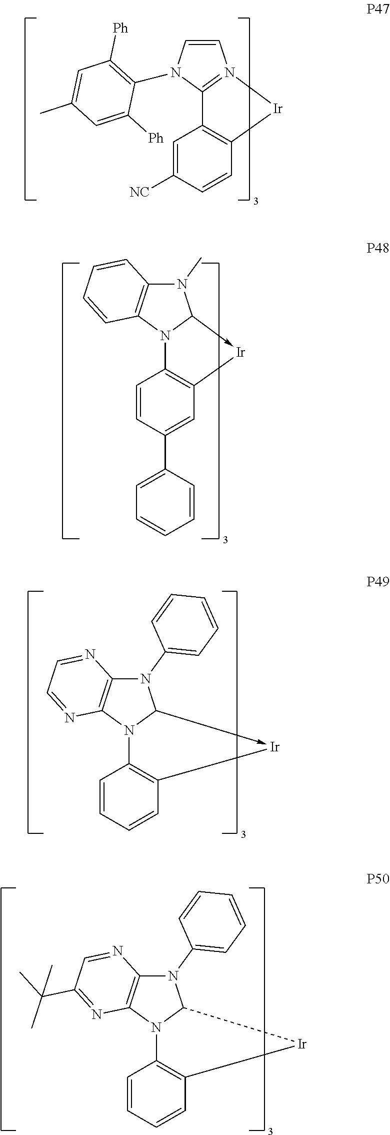

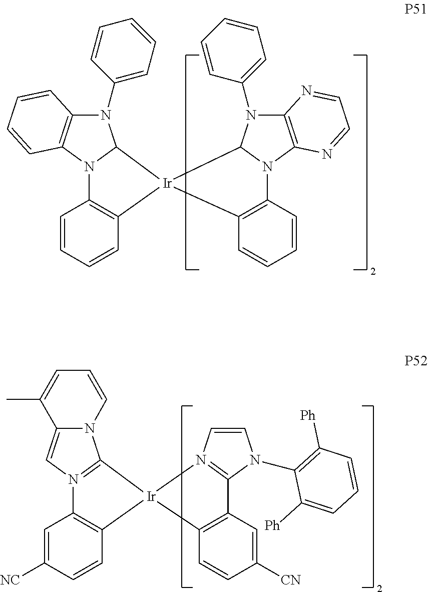

The fluorescent dopant may be, e.g., a polycyclic compound, and may improve the luminous efficiency and life-span characteristics of the light emitting diode by receiving energy transfer within the light emitting layer due to high absorbance. The fluorescent dopant may be, e.g., a condensed polycyclic compound including bromine (B) or nitrogen (N), and may be selected from, e.g., Compounds D1 to D30, below.

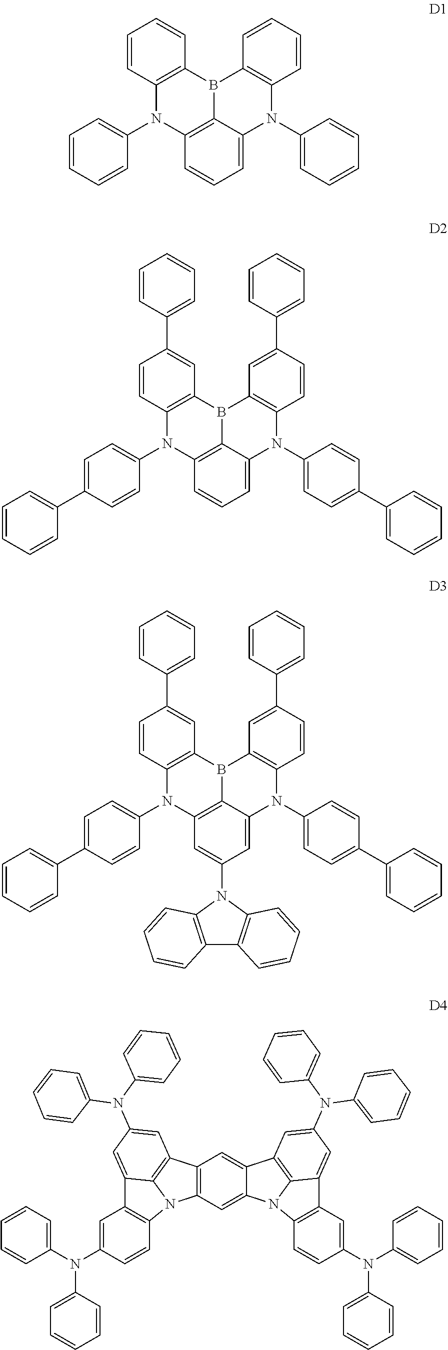 [SMILES: c1ccc(N2c3ccccc3B3c4ccccc4N(c4ccccc4)c4cccc2c43)cc1 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1 ; c1ccc(N(c2ccccc2)c2ccc3c(c2)c2cc(N(c4ccccc4)c4ccccc4)cc4c5cc6c7cc(N(c8ccccc8)c8ccccc8)cc8c9cc(N(c%10ccccc%10)c%10ccccc%10)ccc9n(c6cc5n3c24)c87)cc1]

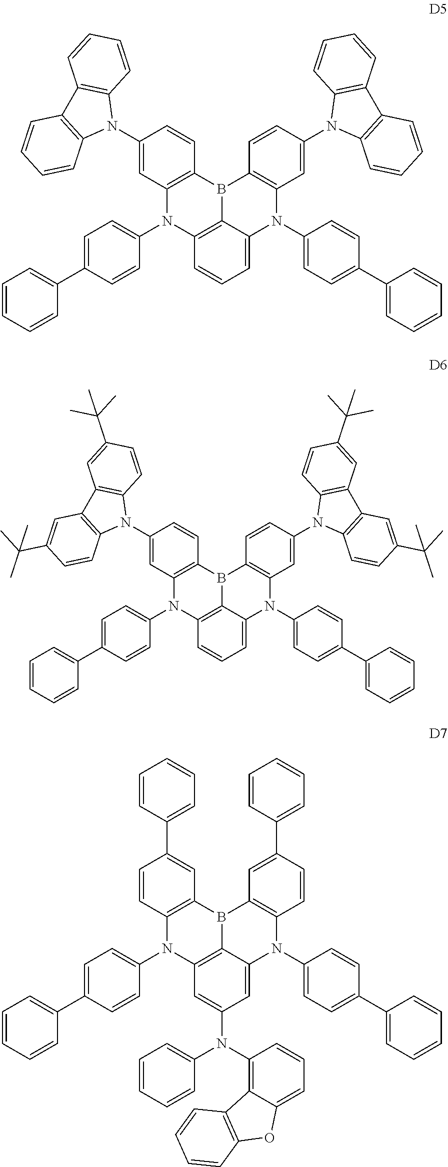 [SMILES: c1ccc(-c2ccc(N3c4cc(-n5c6ccccc6c6ccccc65)ccc4B4c5ccc(-n6c7ccccc7c7ccccc76)cc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ccc(-n2c4ccc(C(C)(C)C)cc4c4cc(C(C)(C)C)ccc42)cc1N3c1ccc(-c2ccccc2)cc1 ; c1ccc(-c2ccc(N3c4ccc(-c5ccccc5)cc4B4c5cc(-c6ccccc6)ccc5N(c5ccc(-c6ccccc6)cc5)c5cc(N(c6ccccc6)c6cccc7oc8ccccc8c67)cc3c54)cc2)cc1]

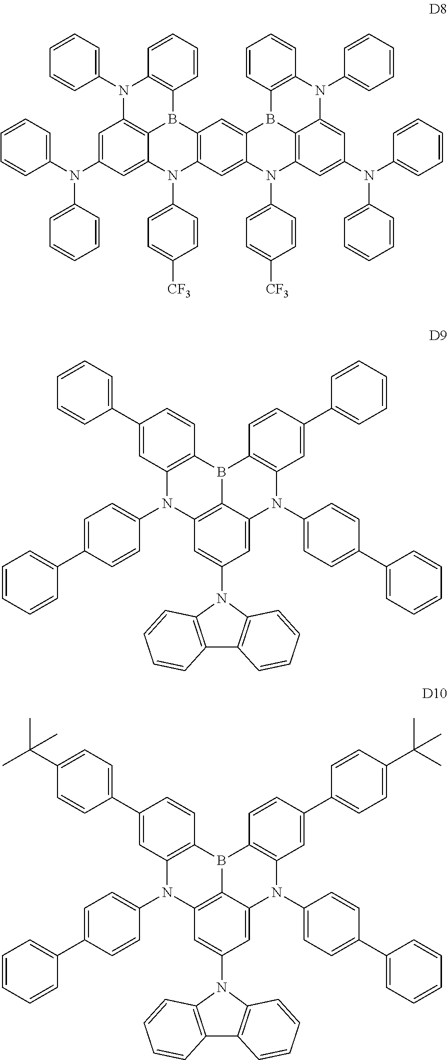 [SMILES: FC(F)(F)c1ccc(N2c3cc4c(cc3B3c5ccccc5N(c5ccccc5)c5cc(N(c6ccccc6)c6ccccc6)cc2c53)B2c3ccccc3N(c3ccccc3)c3cc(N(c5ccccc5)c5ccccc5)cc(c32)N4c2ccc(C(F)(F)F)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-n6c7ccccc7c7ccccc76)cc3c54)cc2)cc1 ; CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1]

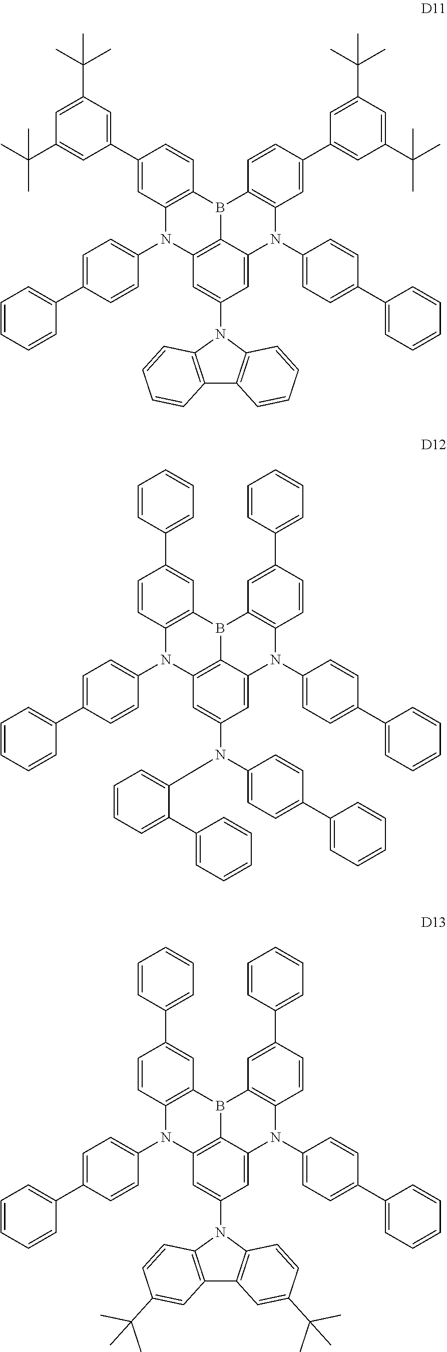 [SMILES: CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1 ; c1ccc(-c2ccc(N(c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c3ccccc3-c3ccccc3)cc2)cc1 ; CC(C)(C)c1ccc2c(c1)c1cc(C(C)(C)C)ccc1n2-c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1]

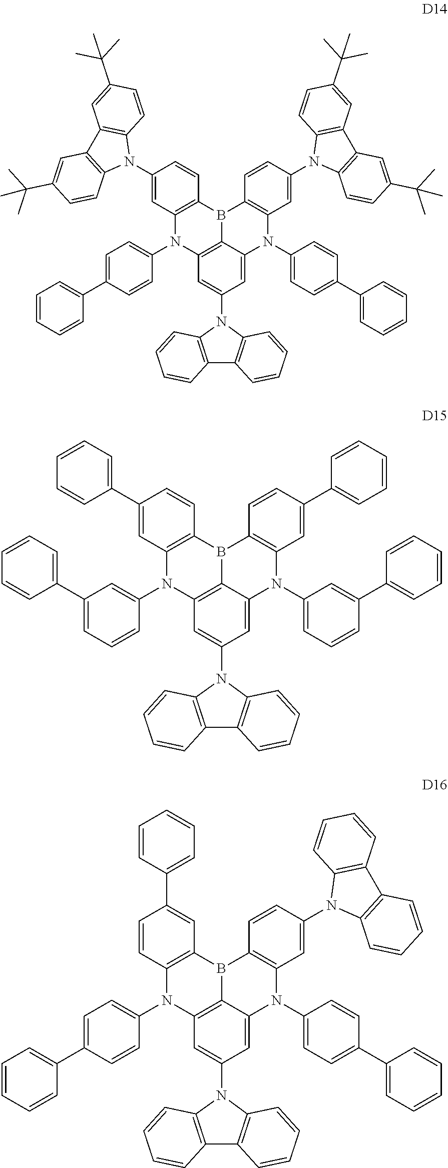 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccccc2)cc1 ; c1ccc(-c2cccc(N3c4cc(-c5ccccc5)ccc4B4c5ccc(-c6ccccc6)cc5N(c5cccc(-c6ccccc6)c5)c5cc(-n6c7ccccc7c7ccccc76)cc3c54)c2)cc1 ; c1ccc(-c2ccc(N3c4ccc(-c5ccccc5)cc4B4c5ccc(-n6c7ccccc7c7ccccc76)cc5N(c5ccc(-c6ccccc6)cc5)c5cc(-n6c7ccccc7c7ccccc76)cc3c54)cc2)cc1]

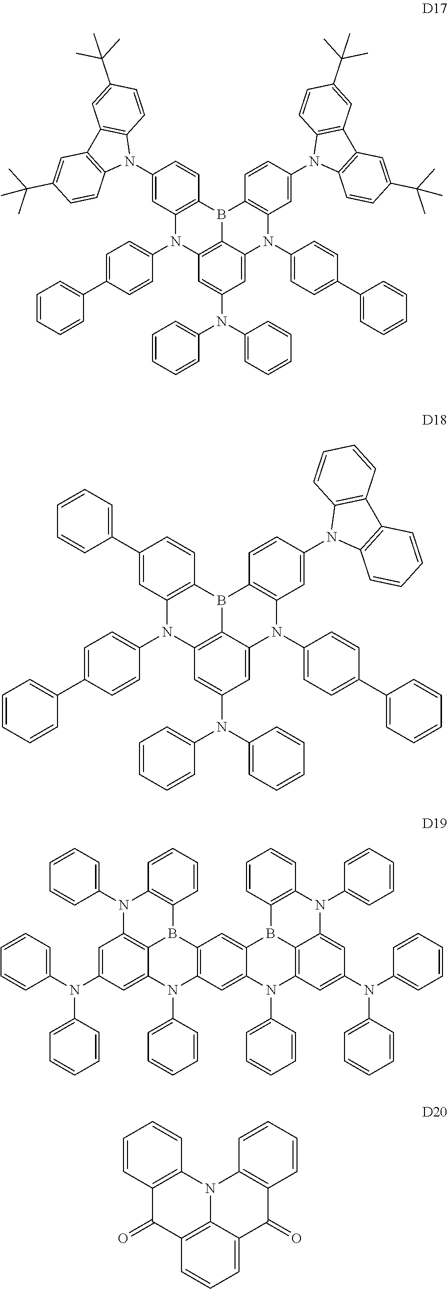 [SMILES: CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1ccc(-n2c4ccc(C(C)(C)C)cc4c4cc(C(C)(C)C)ccc42)cc1N3c1ccc(-c2ccccc2)cc1 ; c1ccc(-c2ccc(N3c4cc(-c5ccccc5)ccc4B4c5ccc(-n6c7ccccc7c7ccccc76)cc5N(c5ccc(-c6ccccc6)cc5)c5cc(N(c6ccccc6)c6ccccc6)cc3c54)cc2)cc1 ; c1ccc(N(c2ccccc2)c2cc3c4c(c2)N(c2ccccc2)c2cc5c(cc2B4c2ccccc2N3c2ccccc2)B2c3ccccc3N(c3ccccc3)c3cc(N(c4ccccc4)c4ccccc4)cc(c32)N5c2ccccc2)cc1 ; O=c1c2ccccc2n2c3ccccc3c(=O)c3cccc1c32]

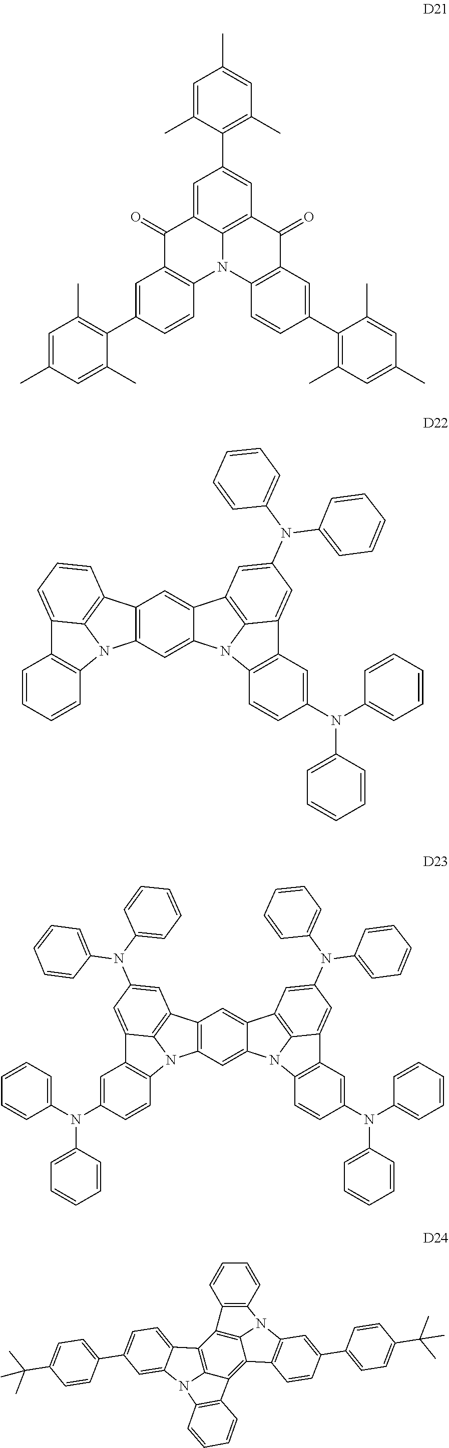

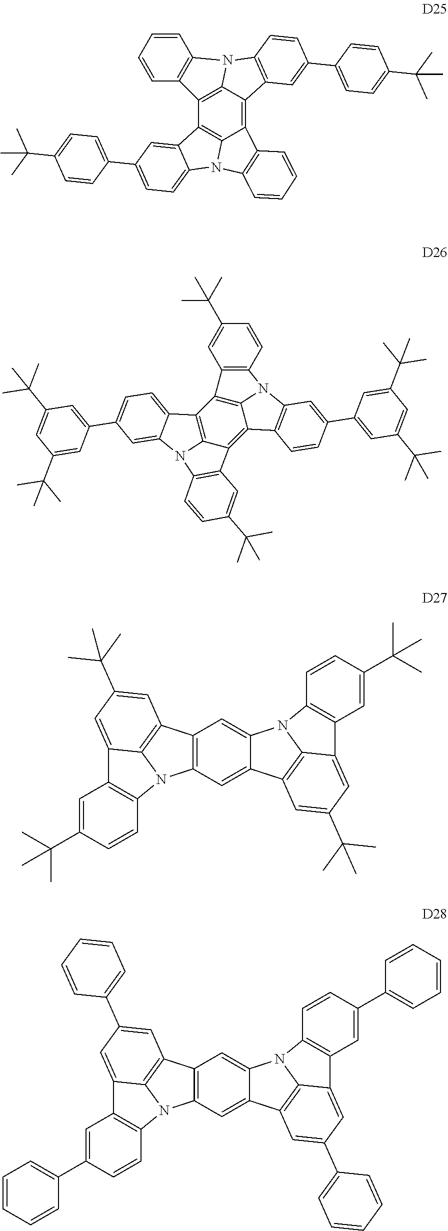

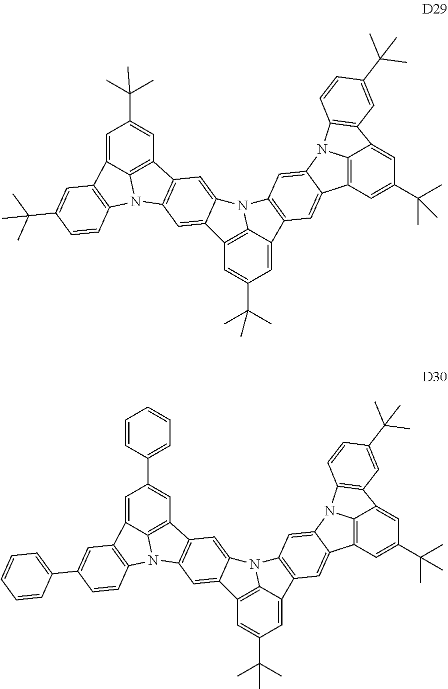 [SMILES: CC(C)(C)c1ccc2c(c1)c1cc(C(C)(C)C)cc3c4cc5c(cc4n2c13)c1cc(C(C)(C)C)cc2c3cc4c6cc(C(C)(C)C)cc7c8cc(C(C)(C)C)ccc8n(c4cc3n5c12)c76 ; CC(C)(C)c1ccc2c(c1)c1cc(C(C)(C)C)cc3c4cc5c6cc(C(C)(C)C)cc7c8cc9c(cc8n(c5cc4n2c13)c76)c1cc(-c2ccccc2)cc2c3cc(-c4ccccc4)ccc3n9c21]

The phosphorescent sensitizer and the fluorescent dopant may each be included in an amount of less than or equal to about 20 wt %, e.g., about 0.1 to about 20 wt %, about 0.1 to about 15 wt %, about 0.1 to about 10 wt %, about 0.1 to about 7 wt %, about 0.1 to about 5 wt %, about 0.1 to about 4 wt %, about 1 to about 20 wt %, about 1 to about 15 wt %, about 1 to about 10 wt %, about 1 to about 7 wt %, about 1 to about 5 wt %, or about 1 to about 4 wt %, based on a total amount of the composition for an organic optoelectronic device.

The composition for an organic optoelectronic device may further include an additive, and the additive may be an organic material, an inorganic material, an organic/inorganic material, or a combination thereof.

Hereinafter, an organic optoelectronic device using the aforementioned composition for an organic optoelectronic device will be described.

The organic optoelectronic device may be, e.g., an organic light emitting diode, an organic photoelectric device, or an organic solar cell. For example, an organic optoelectronic device may be an organic light emitting diode.

The organic optoelectronic device may include an anode and a cathode facing each other, and an organic layer between the anode and the cathode, and the organic layer may include the aforementioned composition. The organic layer may include an active layer (e.g., a light emitting layer or a light absorbing layer), and the aforementioned composition may be included in the active layer. The organic layer may include an auxiliary layer between the anode and the active layer and/or between the cathode and the active layer, and the aforementioned composition may be included in the auxiliary layer.

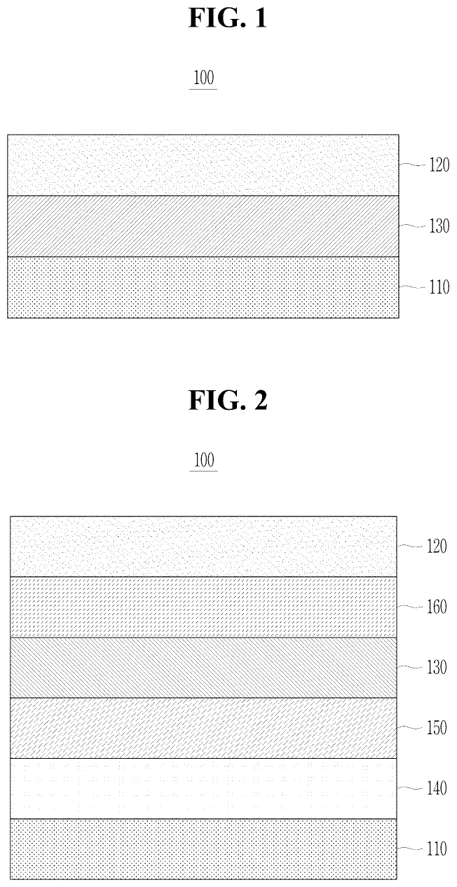

FIG. 1 is a cross-sectional view showing an example of an organic light emitting diode, which is an example of an organic optoelectronic device according to some embodiments.

Referring to FIG. 1, an organic light emitting diode 100 according to some embodiments may include an anode 110 and a cathode 120 facing each other, and a light emitting layer 130 between the anode 110 and the cathode 120. The light emitting layer 130 may include the aforementioned composition.

The anode 110 may be made of a conductor with a high work function to facilitate hole injection, and may be made, e.g., of a metal, a metal oxide, and/or a conductive polymer. The anode 110 may be made of a metal such as nickel, platinum, vanadium, chromium, copper, zinc, gold, or an alloy thereof; a metal oxide such as zinc oxide, indium oxide, indium tin oxide (ITO), and indium zinc oxide (IZO); a combination of a metal and an oxide such as ZnO and Al or SnO₂ and Sb; and/or a conductive polymer such as poly(3-methylthiophene), poly(3,4-(ethylene-1,2-dioxy)thiophene)(PEDOT), polypyrrole, and polyaniline.

The cathode 120 may be made of a conductor with a low work function to facilitate electron injection, and may be made of, e.g., a metal, a metal oxide, and/or a conductive polymer. The cathode 120 may be made of a metal such as magnesium, calcium, sodium, potassium, titanium, indium, yttrium, lithium, gadolinium, aluminum, silver, tin, lead, cesium, barium, or an alloy thereof; and/or a multilayer structure material such as LiF/Al, LiO₂/Al, LiF/Ca, and BaF₂/Ca.

The light emitting layer 130 may include the aforementioned composition for organic optoelectronic devices as a mixed host. The light emitting layer 130 may further include another organic compound as a mixed host. The light emitting layer 130 may further include the aforementioned light-emitting dopant and may include a fluorescent dopant, a phosphorescent sensitizer, or a combination thereof as described above. As an example, the light emitting layer 130 may emit light in a blue light-emitting spectrum by combining the aforementioned composition for an organic optoelectronic device and a light-emitting dopant. At least one of the aforementioned first and second compounds of the composition for an organic optoelectronic device has a high triplet energy level of greater than or equal to about 2.8 eV, so that exciton transfer to the blue light-emitting dopant may be easy, and thus an organic optoelectronic device having high-efficiency and long life-span may be realized. For example, the peak wavelength of the blue emission spectrum may fall within about 410 nm to about 480 nm, e.g., about 420 nm to about 470 nm or about 430 nm to about 470 nm.

As described above, the composition for an organic optoelectronic device may include a first compound, which is a compound having relatively strong hole transport characteristics, and a second compound, which is a compound having relatively strong electron transport characteristics, so that luminous efficiency may be increased by increasing a balance of electrons and holes, and at the same time life-span may be improved by reducing the non-bonding charges due to the imbalance in the mobility of electrons and holes, compared to the case where the first compound alone or the second compound alone is used.

In detail, in the organic light emitting diode 100 including the light emitting layer 130 using the composition for organic optoelectronic devices as a mixed host, the holes and electrons injected from the anode 110 and the cathode 120 may appropriately be distributed within the light emitting layer 130, the mobility may be controlled to an appropriate level, and exciton generation within the light emitting layer 130 may be strongly induced, thereby increasing the light emitting efficiency of the light emitting layer 130.

In addition, by reducing or preventing the generation of excitons at the interface between the light emitting layer 130 and adjacent layers and/or the accumulation of non-bonding charges at the interface between the light emitting layer 130 and adjacent layers, which may be caused by the difference in mobility of holes and electrons injected from the anode 110 and cathode 120, respectively, within the light emitting layer 130, the roll-off phenomenon in which efficiency is rapidly reduced by non-emitting excitons and/or non-bonding charges may be reduced or prevented, and thus the life-span of the organic light emitting diode may be ultimately improved.

The organic light emitting diode 100 may be manufactured by forming the anode 110 or the cathode 120 on a substrate, forming a light emitting layer using dry film forming methods (e.g., vacuum evaporation, sputtering, plasma plating, and ion plating), and forming the cathode 120 or the anode 110 thereon.

FIG. 2 is a cross-sectional view showing another example of an organic light emitting diode, which is an example of an organic optoelectronic device according to some embodiments.

Referring to FIG. 2, the organic light emitting diode 100 according to the present embodiments may include the anode 110, the cathode 120, and the light emitting layer 130, similar to the aforementioned embodiments. However, unlike the aforementioned embodiments, the organic light emitting diode 100 according to the present embodiments may further include a hole transport layer 140, a hole transport auxiliary layer 150, and an electron transport layer 160.

The hole transport layer 140 may be between the anode 110 and the light emitting layer 130, and the hole transport auxiliary layer 150 may be between the light emitting layer 130 and the hole transport layer 140. The electron transport layer 160 may be between the cathode 120 and the light emitting layer 130.

The hole transport layer 140 may facilitate hole transfer from the anode 110 to the light emitting layer 130, and may include, e.g., an amine compound. For example, the amine compound may have at least one aryl group and/or heteroaryl group with hole characteristics. For example, the amine compound may be represented by Chemical Formula 6a or 6b, but is not limited thereto.

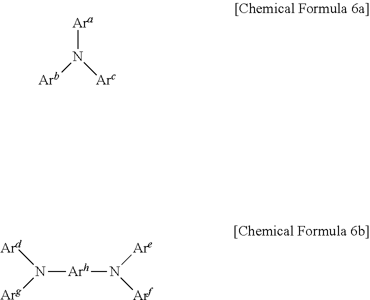

In Chemical Formula 6a or 6b, Ar^{a} to Ar^{g} may be each independently hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof, at least one of Ar^{a} to Ar^{c} and at least one of Ar^{d} to Ar^{g} may be a substituted or unsubstituted C6 to C30 aryl group, a substituted or unsubstituted C2 to C30 heteroaryl group, or a combination thereof, and Ar^{h} may be a single bond, a substituted or unsubstituted C1 to C20 alkylene group, a substituted or unsubstituted C6 to C30 arylene group, a substituted or unsubstituted C2 to C30 heteroarylene group, or a combination thereof.

The hole transport auxiliary layer 150 may form an interface with the light emitting layer 130 by being between the hole transport layer 140 and the light emitting layer 130 and in contact with the light emitting layer 130. The hole transport auxiliary layer 150 may further reduce or prevent the generation of excitons at the interface between the aforementioned light emitting layer 130 and adjacent layers and/or the accumulation of non-bonding charges at the interface between the light emitting layer 130 and adjacent layers, thereby further reducing or preventing the roll-off phenomenon in which efficiency is rapidly reduced due to non-emitting excitons and/or non-bonding charges, and thus ultimately further improving the life-span of the organic optoelectronic device 100.

The electron transport layer 160 may further increase electron injection and/or electron mobility and block holes between the cathode 120 and the light emitting layer 130. The electron transport layer 160 may include, e.g., one or two or more compounds selected from the compounds listed in Group 5, below.

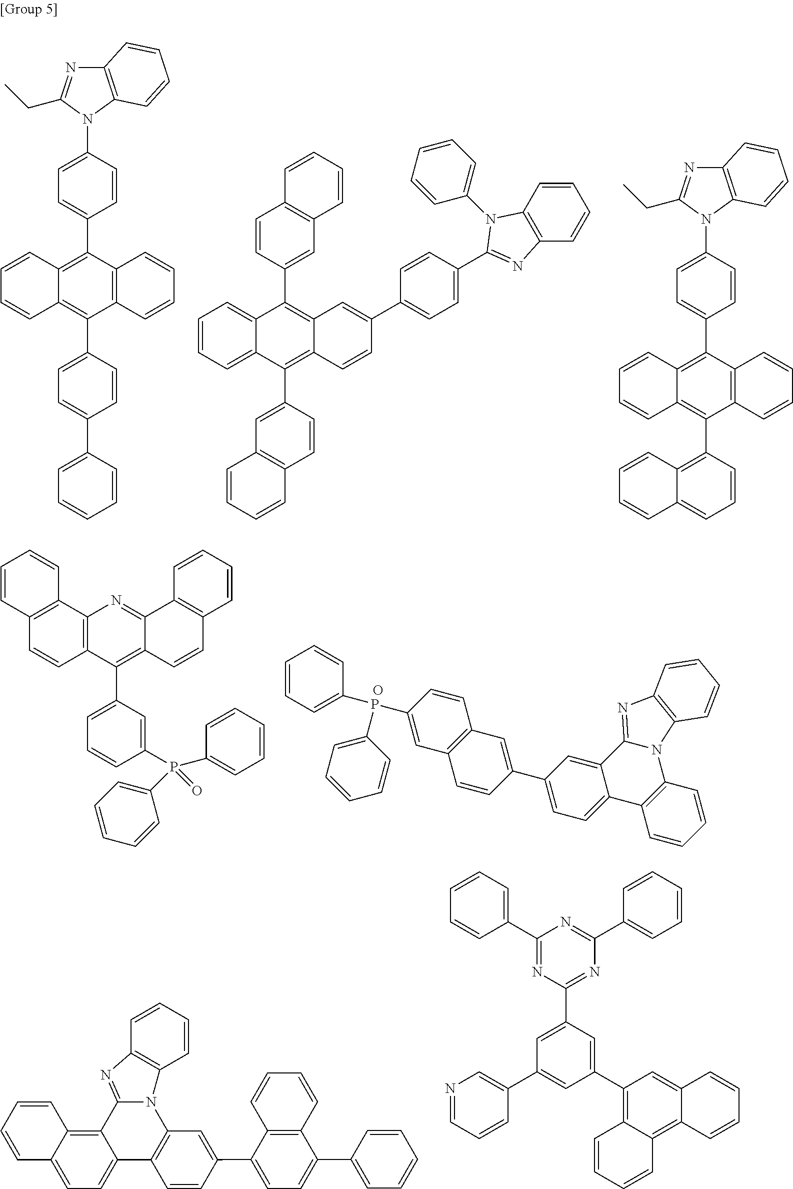

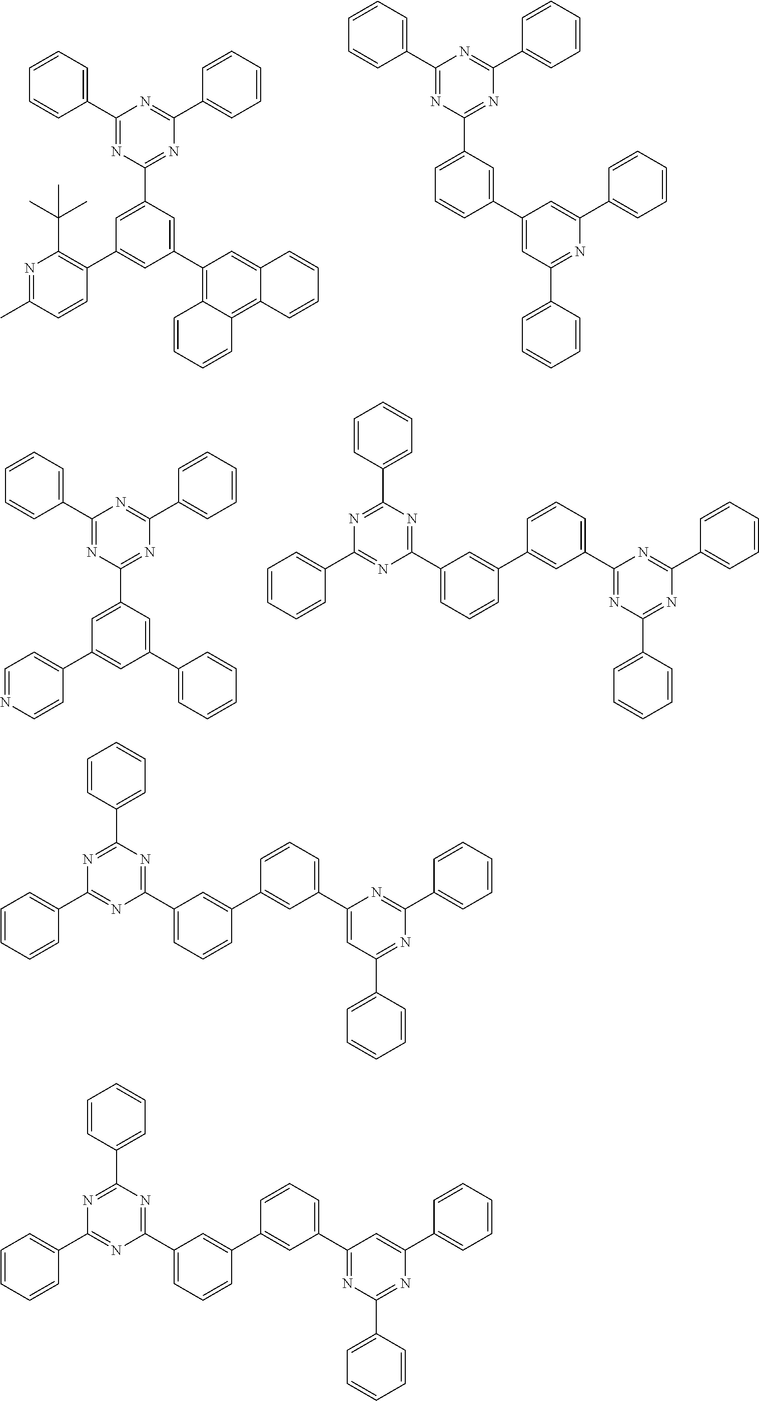 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

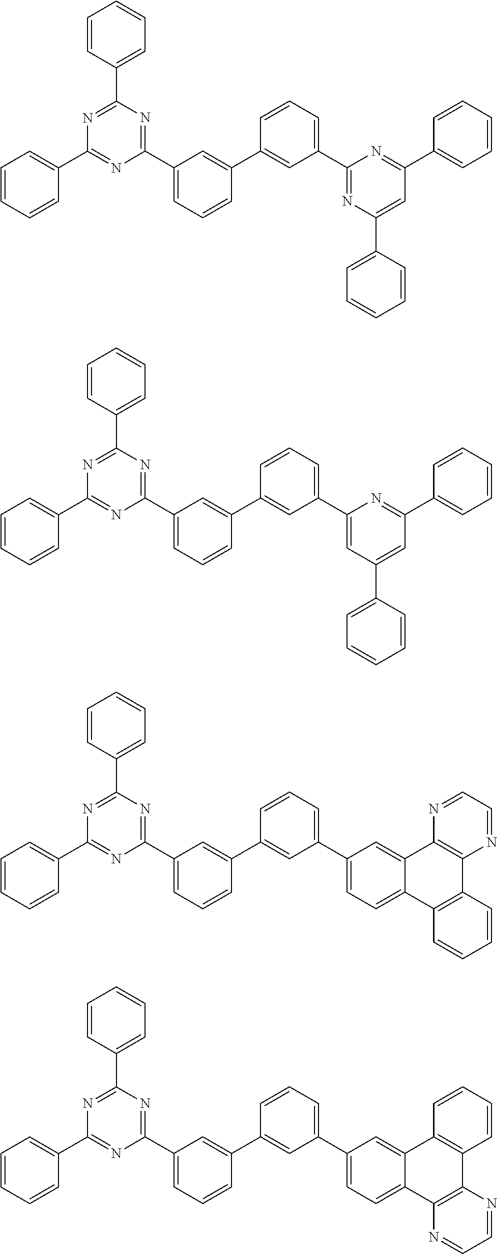 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

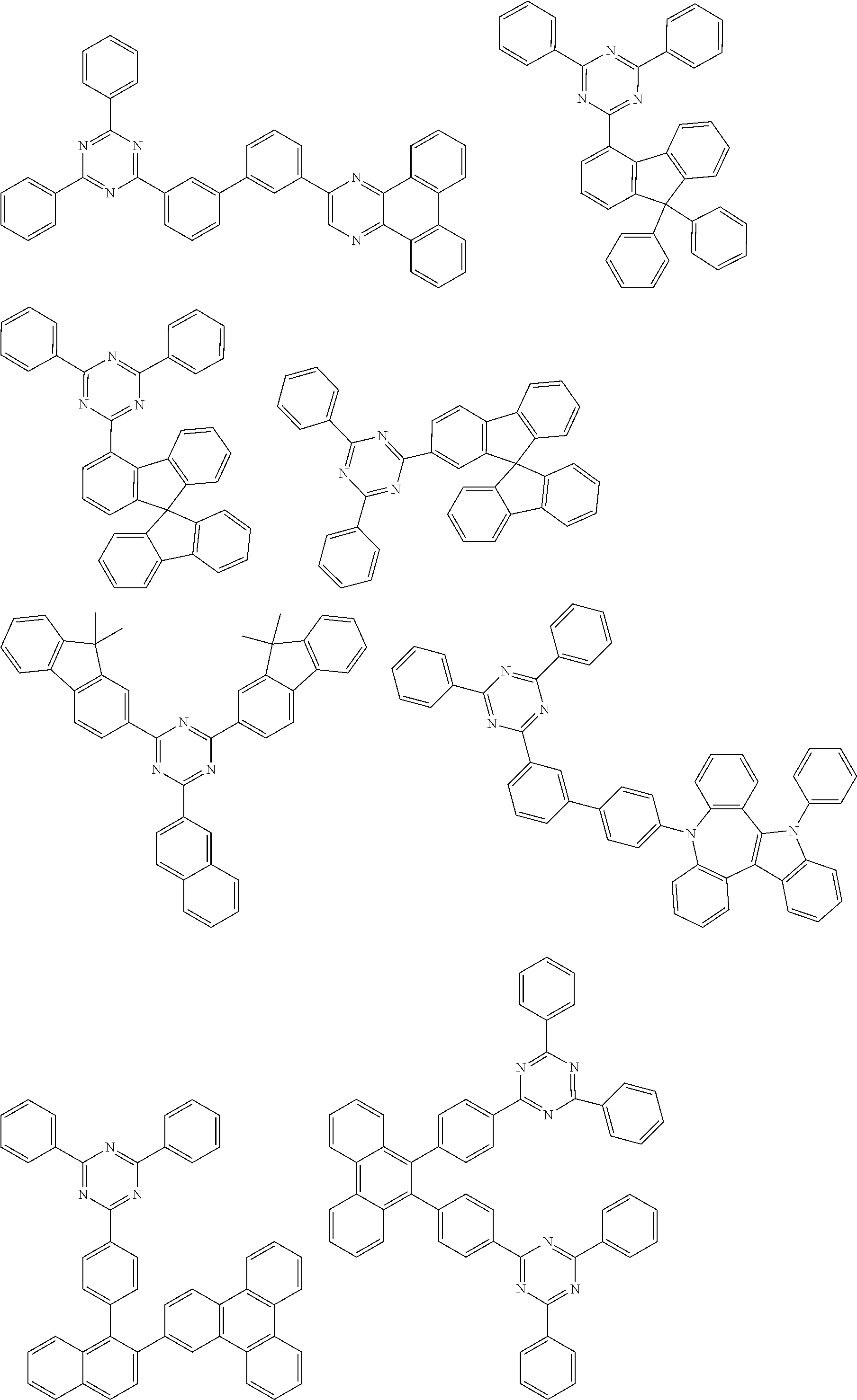

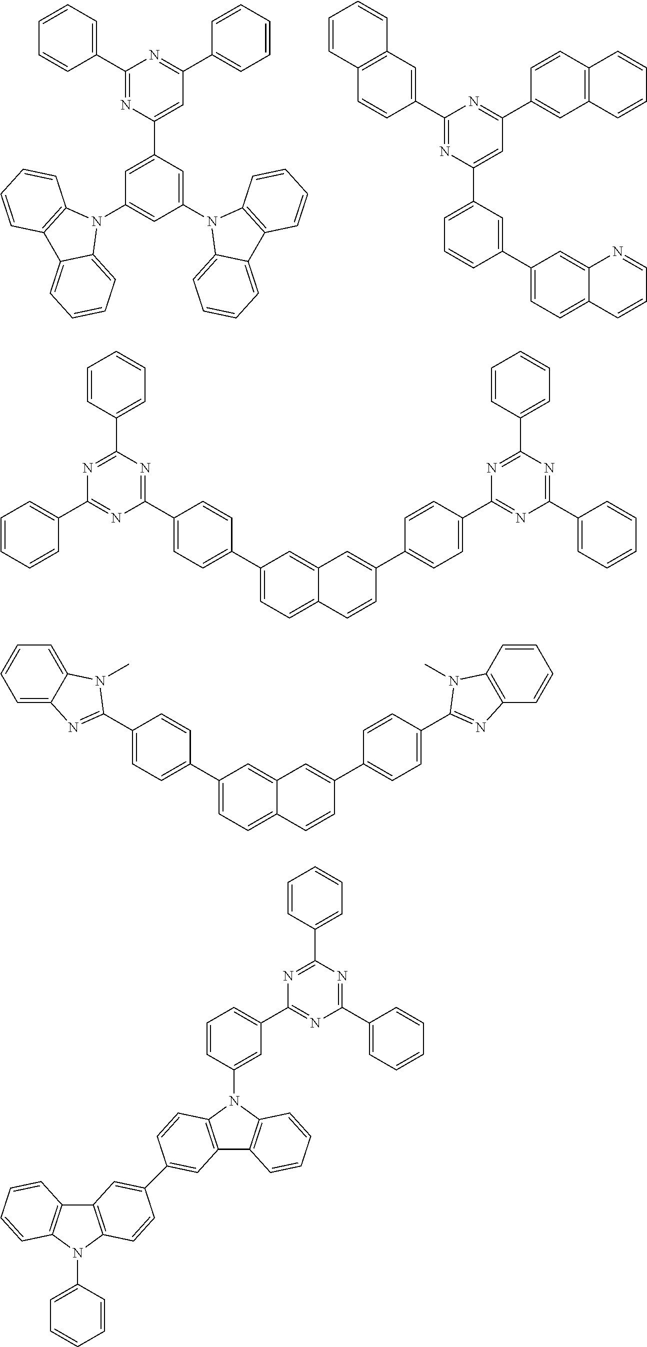

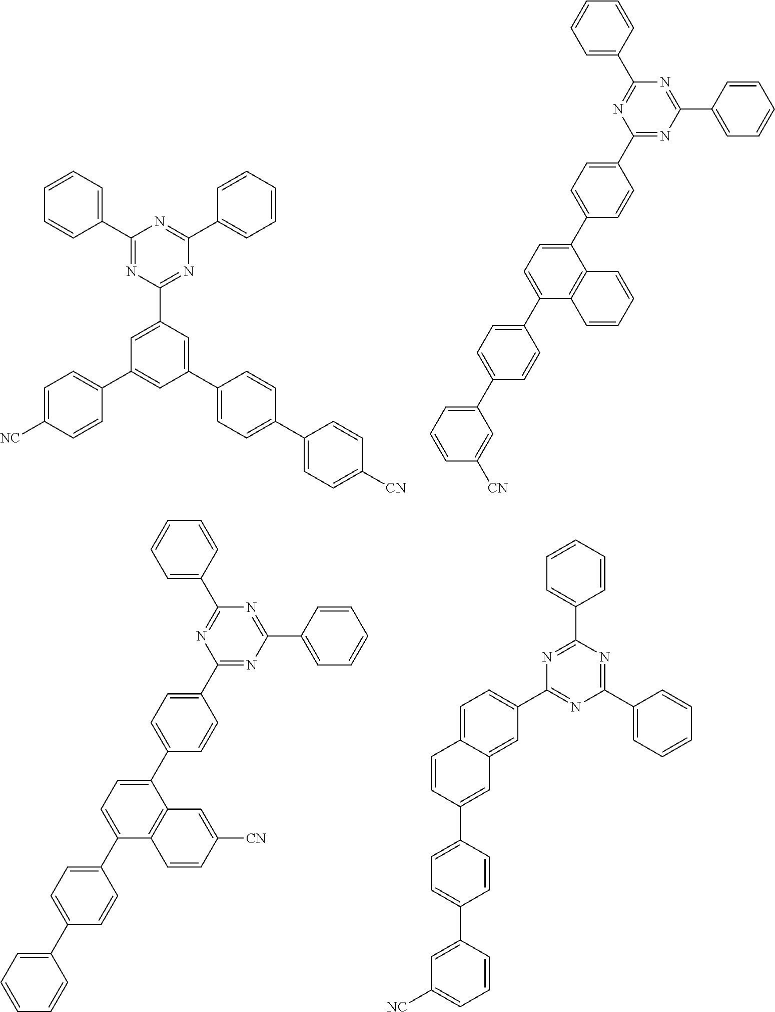 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1]

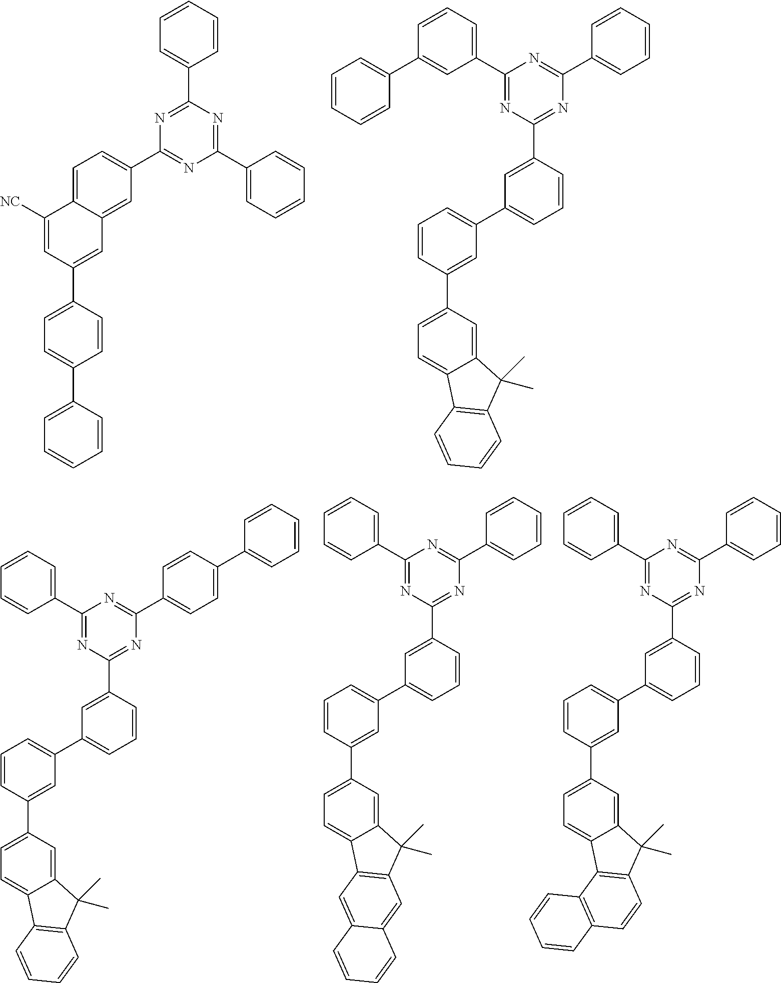

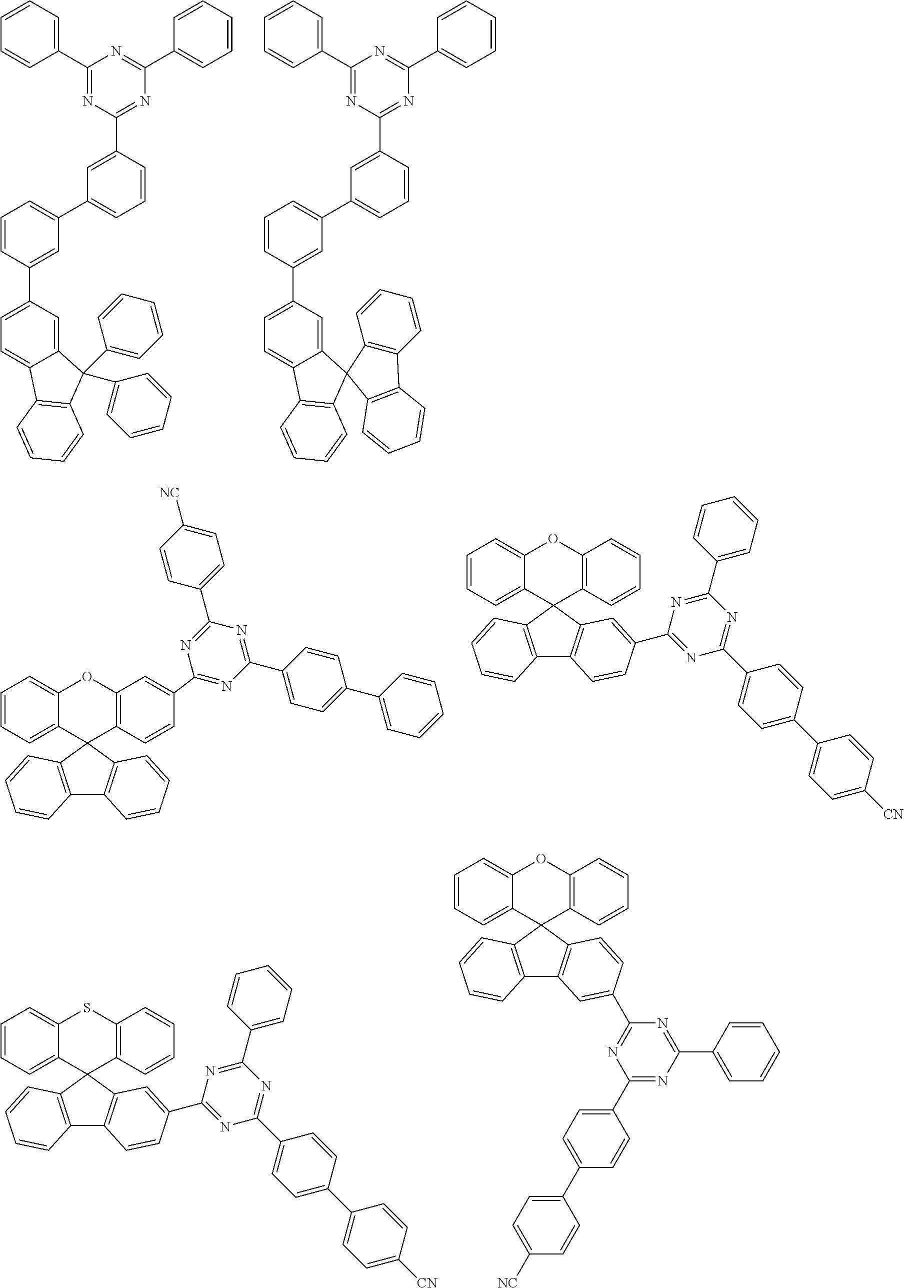

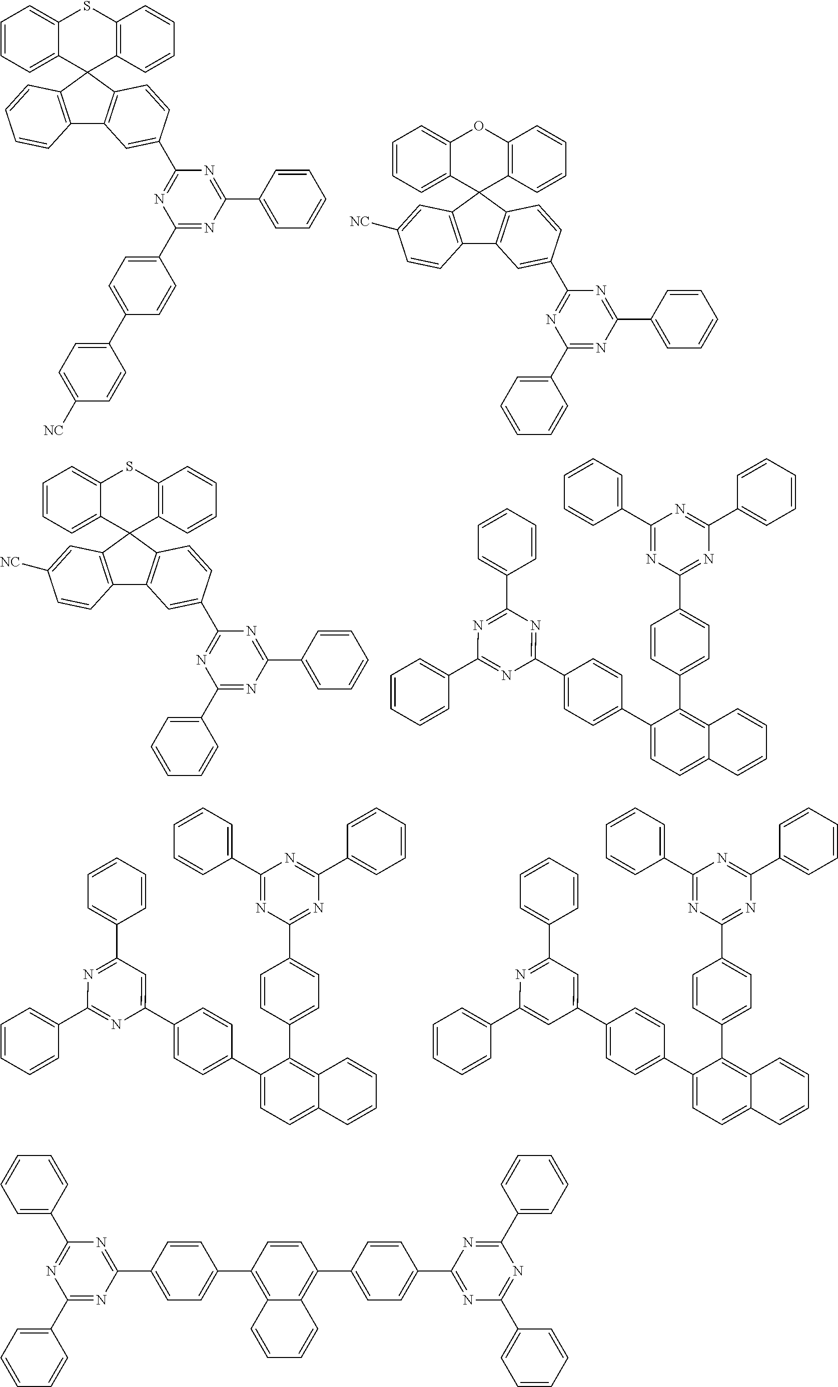

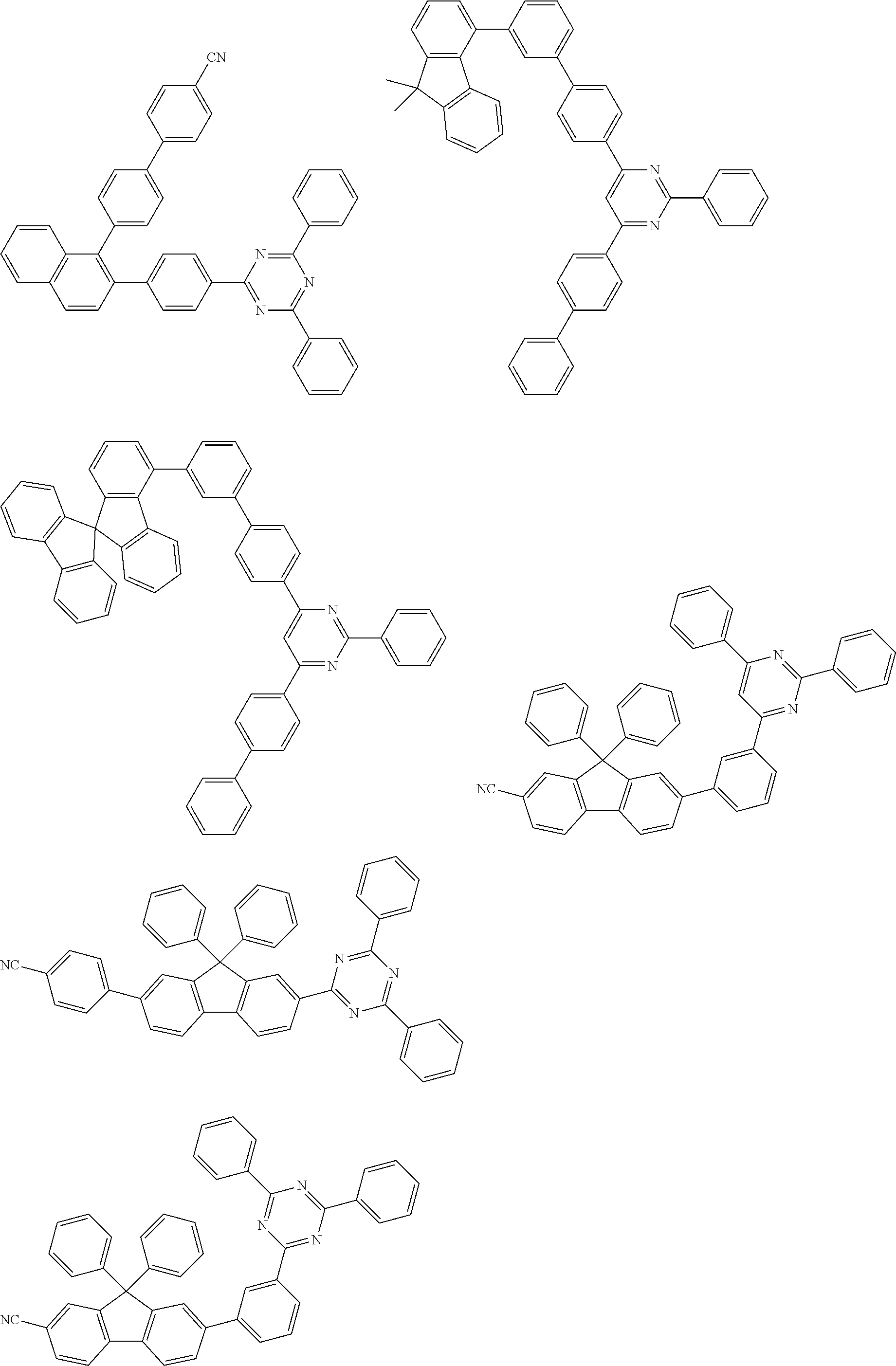

The organic optoelectronic device, including the aforementioned organic light emitting diodes, may be applied to display devices.

Hereinafter, the embodiments are illustrated in more detail with reference to examples. The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry or P&H tech as far as there is no particular comment or were synthesized by known methods.

The compound as one specific examples of embodiments was synthesized through the following steps.

### Synthesis of First Compound

### Synthesis Example 1: Synthesis of Compound H-9

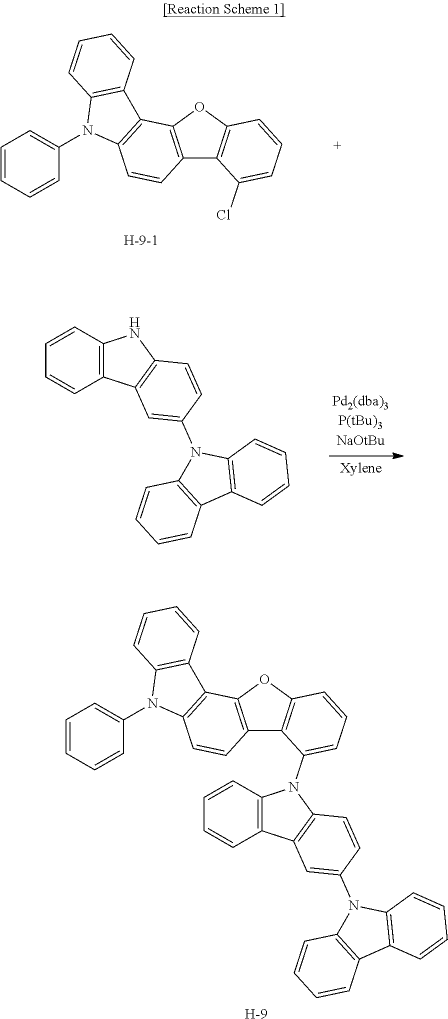

Intermediate H-9-1 (3.0 g, 8.2 mmol), 9H-3,9′-bicarbazole (3.0 g, 9.0 mmol), tris(dibenzylideneacetone) dipalladium (0) Pd₂(dba)₃)(0.4 g, 0.4 mmol), sodium tert-butoxide (NaOtBu)(1.2 g, 12.2 mmol), and tri (tert-butyl)phosphine (P(tBu)₃)(0.2 g, 1.2 mmol) were added to 41 mL of xylene and then stirred under reflux for 12 hours. When a reaction was completed, the resultant was purified through column chromatography (dichloromethane: n-hexane) to obtain 3.4 g (a yield: 62.3%) of Compound H-9.

### Synthesis Example 2: Synthesis of Compound H-33

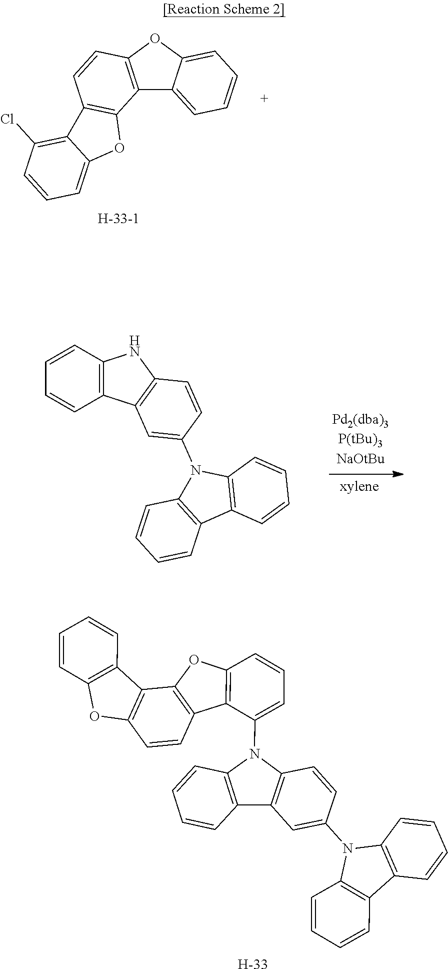

3.8 g (a yield: 63.5%) of Compound H-33 was synthesized in the same manner as in Synthesis Example 1 except that Intermediate H-33-1 was used instead of Intermediate H-9-1.

### Comparative Synthesis Example 1: Synthesis of Compound F-1

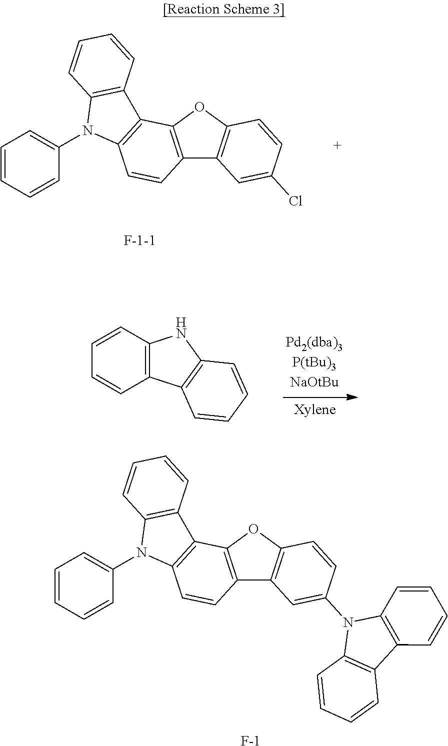

2.7 g (a yield: 67.2%) of Compound F-1 was synthesized in the same manner as in Synthesis Example 1 except that Intermediate F-1-1 and carbazole were used instead of Intermediate H-9-1 and the 9H-3,9′-bicarbazole.

### Comparative Synthesis Example 2: Synthesis of Compound F-2

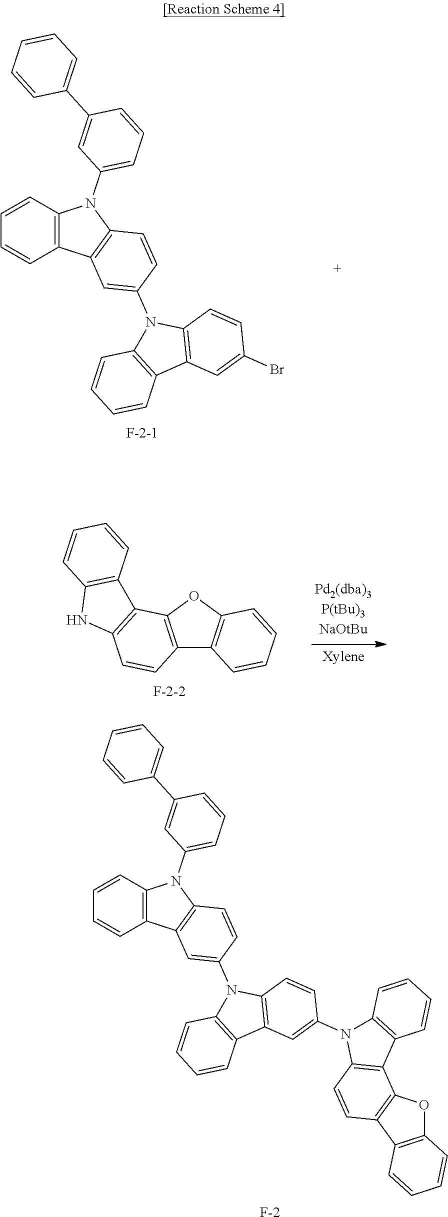

3.3 g (a yield: 83.6%) of Compound F-2 was synthesized in the same manner as in Synthesis Example 1 except that Intermediate F-2-1 and Intermediate F-2-2 were used instead of Intermediate H-9-1 and the 9H-3,9′-bicarbazole.

### Manufacture of Organic Light Emitting Diode I

### Example 1

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then moved to a plasma cleaner, cleaned by using oxygen plasma for 10 minutes, and moved to a vacuum depositor. The prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 600 Å to form a hole transport layer. mCP was deposited to a thickness of 100 Å on the hole transport layer to form a hole transport auxiliary layer. On the hole transport auxiliary layer, Compounds H-9 (host) and P-31 (dopant) obtained in Synthesis Example 1 were vacuum-deposited to form a 400 Å-thick light emitting layer. Subsequently, BCP was deposited on the light emitting layer to a thickness of 50 Å to form an electron transport auxiliary layer, and Compound B and LiQ were simultaneously vacuum deposited in a weight ratio of 1:1 to form a 300 Å-thick electron transport layer. An organic light emitting diode was manufactured by sequentially vacuum depositing 10 Å of LiQ and 1200 Å of Al on the electron transport layer to form a cathode.

Compound A: N-(9,9-diphenyl-9H-fluoren-2-yl)-N,9-diphenyl-9H-carbazol-2-amine

Compound B: 8-{4-[bis (naphthalene-2-yl)-1,3,5-triazin-2-yl]phenyl} quinoline

### Example 2

An organic light emitting diode was manufactured in the same manner as in Example 1 except that Compound H-33 of Synthesis Example 2 instead of Compound H-9 of Synthesis Example 1 was used as the host of the light emitting layer.

### Comparative Example 1

An organic light emitting diode was manufactured in the same manner as in Example 1 except that Compound F-1 of Comparative Synthesis Example 1 instead of Compound H-9 of Synthesis Example 1 was used as the host of the light emitting layer.

### Comparative Example 2

An organic light emitting diode was manufactured in the same manner as in Example 1 except that Compound F-2 of Comparative Synthesis Example 2 instead of Compound H-9 of Synthesis Example 1 was used as the host of the light emitting layer.

### Evaluation I

The organic light emitting diodes according to Examples and Comparative Examples were evaluated with respect to luminous efficiency and life-span characteristics.

The specific measurement method is as follows, and the results are in Table 1.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

The luminance and the current density measured from (1) and (2) above and a voltage were used to calculate the current efficiency (cd/A) at the same current density (10 mA/cm²). Luminous efficiency of the organic light emitting diodes of the examples was calculated as a relative value to that of Comparative Example 1, and the results are shown in Table 1.

### (4) Measurement of Life-Span

Time that the current efficiency (cd/A) of the organic light emitting diodes according to the examples and the comparative examples decreased to 95%, while maintaining the luminance (cd/m²) at 2000 cd/m², was measured. The measured life-spans of the organic light emitting diodes according to the examples and the comparative examples were calculated as relative values based on Comparative Example 1 and are shown in Table 1.

TABLE 1

| | Driving voltage (V) | Life-span (%) |
|---|---|---|
| Example 1 | 4.2 V | 270% |
| Example 2 | 4.4 V | 130% |
| Comparative Example 1 | 4.6 V | 100% |
| Comparative Example 2 | 4.2 V | 91% |

Referring to Table 1, the organic light emitting diodes of the examples, compared with the organic light emitting diode of Comparative Example 1, exhibited improved driving voltage and life-span characteristics and compared with the organic light emitting diode of Comparative Example 2, exhibited an equivalent driving voltage and improved life-span characteristics.

### Synthesis of Second Compound

### Synthesis Example 3: Synthesis of Compound E-284

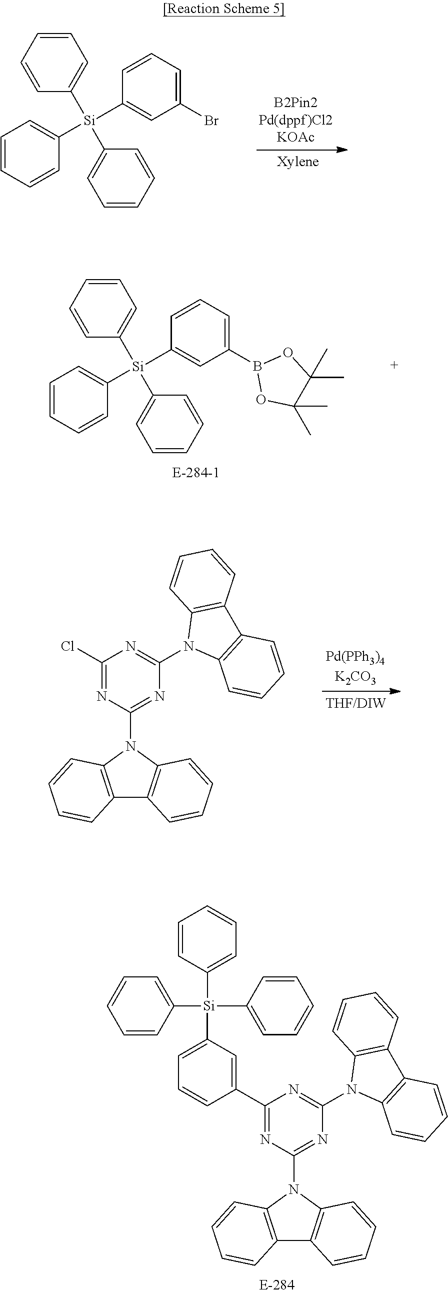 [SMILES: Brc1cccc([Si](c2ccccc2)(c2ccccc2)c2ccccc2)c1 ; CC1(C)OB(c2cccc([Si](c3ccccc3)(c3ccccc3)c3ccccc3)c2)OC1(C)C ; Clc1nc(-n2c3ccccc3c3ccccc32)nc(-n2c3ccccc3c3ccccc32)n1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

First step: Synthesis of Intermediate E-284-1

(3-bromophenyl)triphenylsilane (3.0 g, 7.2 mmol), 4,4,4′,4′,5,5,5′,5′-octamethyl-2,2′-bi (1,3,2-dioxaborolane)(B2Pin2)(2.4 g, 9.4 mmol), [1,1′-bis(diphenylphosphino) ferrocene]dichloropalladium (II)(Pd (dppf) C12)(0.3 g, 0.4 mmol), and potassium acetate (KOAc)(2.1 g, 21.7 mmol) were added to 36 mL of xylene and then stirred under reflux for 12 hours. When a reaction was completed, the resultant was purified through column chromatography (dichloromethane: n-hexane) to obtain 2.4 g (a yield: 73.2%) of Intermediate E-284-1.

Second step: Synthesis of Compound E-284

Intermediate E-284-1 (2.4 g, 5.2 mmol), 9,9′-(6-chloro-1,3,5-triazine-2,4-diyl)bis(9H-carbazole)(2.3 g, 5.2 mmol), Pd(PPh₃)₄ (0.3 g, 0.3 mmol), and potassium carbonate (K₂CO₃)(2.2 g, 15.6 mmol) were added to 17 mL of THF and 8.7 mL of DIW and then stirred under reflux for 12 hours. When a reaction was completed, the resultant was purified through column chromatography (dichloromethane: n-hexane) to obtain 3.5 g (a yield: 89.9%) of Compound E-284.

### Manufacture of Organic Light Emitting Diode II

### Example 3

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then moved to a plasma cleaner, cleaned by using oxygen plasma for 10 minutes, and moved to a vacuum depositor. The prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 600 Å to form a hole transport layer. mCP was deposited to a thickness of 100 Å on the hole transport layer to form a hole transport auxiliary layer. On the hole transport auxiliary layer, Compound H-9 obtained in Synthesis Example 1 and Compound E-284 obtained in Synthesis Example 3 were used simultaneously as hosts, P31 was doped at 13 wt % as a phosphorescent sensitizer, and D3 was doped at 1.5 wt % as a fluorescent dopant to form a 400 Å-thick light emitting layer by vacuum deposition. Herein, Compound A-54 and Compound D-72 were used in a weight ratio of 4:6. Subsequently, BCP was deposited on the light emitting layer to a thickness of 50 Å to form an electron transport auxiliary layer, and Compound B and Liq were simultaneously vacuum deposited in a weight ratio of 1:1 to form a 300 Å-thick electron transport layer. An organic light emitting diode was manufactured by sequentially vacuum depositing 10 Å of LiQ and 1200 Å of Al on the electron transport layer to form a cathode.

ITO/Compound A (3% NDP-9 doping, 100 Å)/Compound A (600 Å)/mCP (100 Å)/EML [Host (Compound H-9: Compound E-284): P31: D3=85.5 wt %: 13 wt %: 1.5 wt %] (400 Å)/BCP (50 Å)/Compound B: LiQ (300 Å)/LiQ (10 Å)/AI (1200 Å).

Compound A: N-(9,9-diphenyl-9H-fluoren-2-yl)-N,9-diphenyl-9H-carbazol-2-amine

Compound B: 8-{4-[bis (naphthalene-2-yl)-1,3,5-triazin-2-yl]phenyl} quinoline

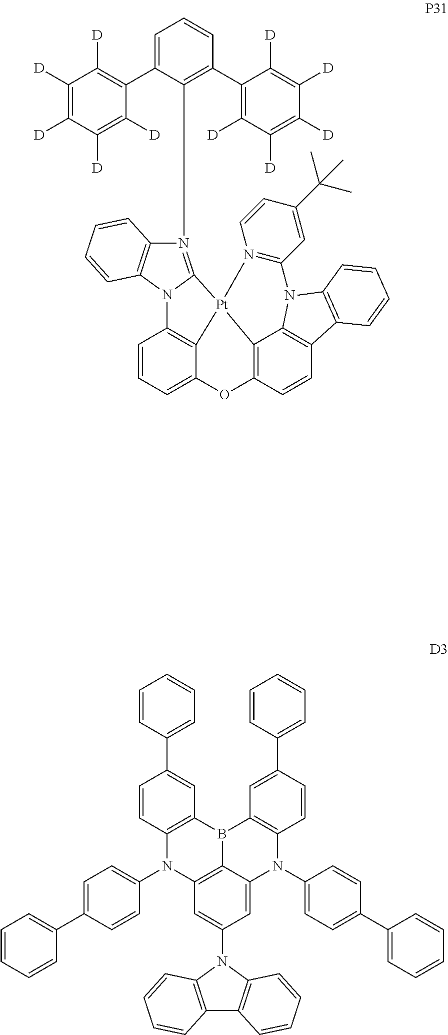

### Example 4

An organic light emitting diode was manufactured in the same manner as in Example 3 except that Compound H-33 of Synthesis Example 2 and Compound E-284 of Synthesis Example 3 were used instead of Compound H-9 of Synthesis Example 1 and Compound E-284 of Synthesis Example 3 as the host of the light emitting layer.

### Comparative Example 3

An organic light emitting diode was manufactured in the same manner as in Example 3 except that Compound F-1 of Comparative Synthesis Example 1 and Compound E-284 of Synthesis Example 3 were used instead of Compound H-9 of Synthesis Example 1 and Compound E-284 of Synthesis Example 3 as the host of the light emitting layer.

### Comparative Example 4

An organic light emitting diode was manufactured in the same manner as in Example 3 except that Compound F-2 of Comparative Synthesis Example 2 and Compound E-284 of Synthesis Example 3 were used instead of Compound H-9 of Synthesis Example 1 and Compound E-284 of Synthesis Example 3 as the host of the light emitting layer.

### Evaluation II

The organic light emitting diodes according to the examples and the comparative examples were evaluated with respect to luminous efficiency and life-span characteristics.

The results are shown in Table 2.

TABLE 2

| | Host | | Driving | |
|---|---|---|---|---|
| | First | Second | voltage | Life-span |
| No. | compound | compound | (V) | (%) |
| Example 3 | H-9 | E-284 | 4.3 V | 309% |
| Example 4 | H-33 | E-284 | 4.5 V | 140% |
| Comparative Example 3 | F-1 | E-284 | 4.8 V | 100% |
| Comparative Example 4 | F-2 | E-284 | 4.2 V | 83% |

Referring to Table 2, the organic light emitting diodes according to the examples, compared with the organic light emitting diode according to that of Comparative Example 3, were confirmed to exhibit improved driving voltage and life-span characteristics, and compared with that of Comparative Example 4, to exhibit improved life-span characteristics.

By way of summation and review, some example embodiments provide a compound for an organic optoelectronic device that may achieve high efficiency and long life-span characteristics. Some example embodiments provide a composition for an organic optoelectronic device including the compound for an organic optoelectronic device. Some example embodiments provide an organic optoelectronic device including the compound or the composition. Some example embodiments provide a display device including the organic optoelectronic device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound represented by Chemical Formula 1: 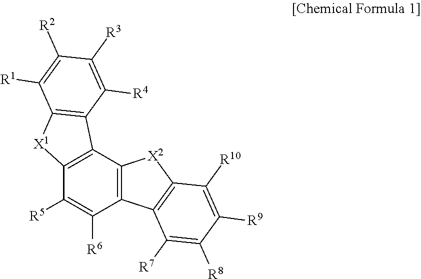
wherein, in Chemical Formula 1,
X¹ and X² are each independently NR^{a}, O, S, Se, or Te,
any one of R¹ to R¹⁰ is a group represented by Chemical Formula A, the remainder of R¹ to R¹⁰ and R^{a} are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and
R¹ to R¹⁰ and R^{a} are each independently present, or two adjacent ones among R¹ to R¹⁰ and R^{a} are linked to form a ring, 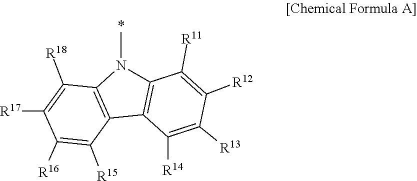
wherein, in Chemical Formula A,
R¹¹ to R¹⁸ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen,
provided that, any one of R¹¹ to R¹⁸ is a substituted or unsubstituted carbazolyl group,
R¹¹ to R¹⁸ are each independently present, or two adjacent ones among R¹¹ to R¹⁸ are linked to form a ring, and
* is a linking point with Chemical Formula 1.

2. The compound as claimed in claim 1, wherein any one of R¹¹ to R¹⁸ of Chemical Formula A is represented by Chemical Formula Aa: 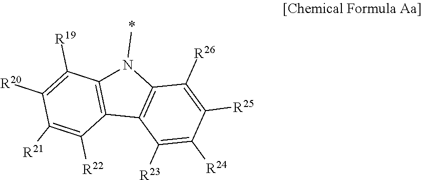
wherein, in Chemical Formula Aa,
R¹⁹ to R²⁶ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen,
R¹⁹ to R²⁶ are each independently present, or two adjacent ones among R¹¹ to R¹⁸ are linked to form a ring, and
* is a linking point with Chemical Formula A.

3. The compound as claimed in claim 2, wherein at least one of R¹⁹ to R²⁶ of Chemical Formula Aa is deuterium.

4. The compound as claimed in claim 1, wherein Chemical Formula A is represented by Chemical Formula A-1: 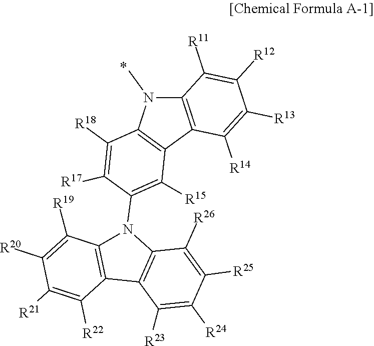
wherein, in Chemical Formula A-1,
R¹¹ to R¹⁵ and R¹⁷ to R²⁶ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen,
R¹¹ to R¹⁵ and R¹⁷ to R²⁶ are each independently present, or two adjacent ones among R¹¹ to R¹⁵ and R¹⁷ to R²⁶ are linked to form a ring, and
* is a linking point with Chemical Formula 1.

5. The compound as claimed in claim 1, wherein any one of R¹ to R⁴ and R⁷ to R¹⁰ of Chemical Formula 1 is a group represented by Chemical Formula A.

6. The compound as claimed in claim 1, wherein:
at least one of X¹ and X² is NR^{a}, and
R^{a} is each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted benzofuranyl group, a substituted or unsubstituted dibenzofuranyl group, a substituted or unsubstituted benzothiophenyl group substituted or unsubstituted dibenzothiophenyl group, a substituted or unsubstituted benzoselenophenyl group, a substituted or unsubstituted dibenzoselenophenyl group, a substituted or unsubstituted benzosilolyl group, or a substituted or unsubstituted dibenzosilolyl group.

7. The compound as claimed in claim 1, wherein at least one of R¹ to R¹⁸ is deuterium.

8. A composition for an organic optoelectronic device, comprising:
the first compound as claimed in claim 1, and
a second compound represented by Chemical Formula 2: 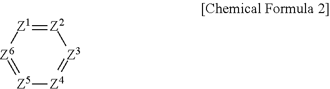
wherein, in Chemical Formula 2,
Z¹ to Z⁶ are each independently N or C-L^{a}-R^{e},
at least two of Z¹ to Z⁶ are N,
L^{a} is each independently a single bond, a substituted or unsubstituted C6 to C20 arylene group, a substituted or unsubstituted divalent C2 to C20 heterocyclic group, or a combination thereof,
R^{e} is each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, and
R^{e} is each independently present, or two adjacent R^{e}s are linked to form a substituted or unsubstituted aliphatic, aromatic or heteroaromatic monocyclic or polycyclic ring.

9. The composition as claimed in claim 8, wherein the second compound represented by Chemical Formula 2 is represented by any one of Chemical Formulas 2A to 2C: 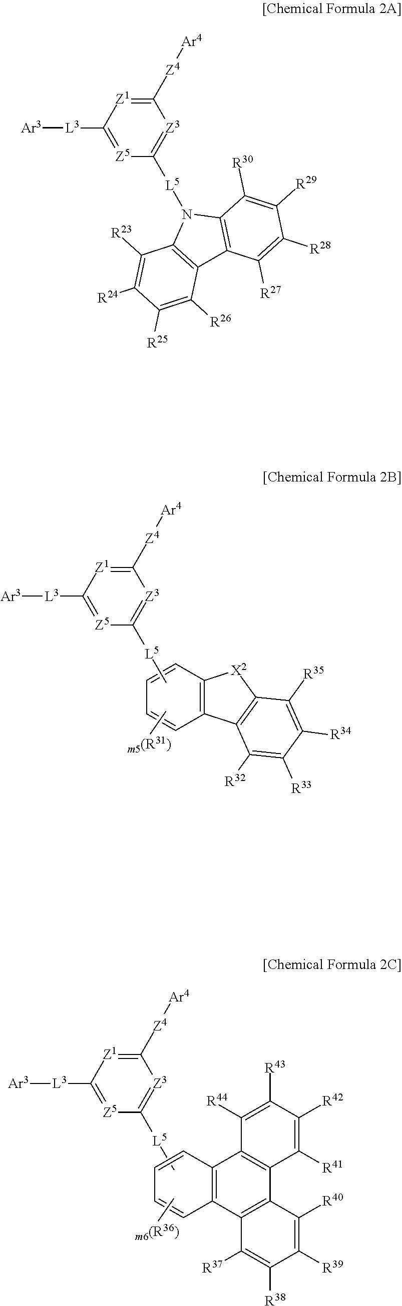
wherein, in Chemical Formula 2A to Chemical Formula 2C,
Z¹, Z³, and Z⁵ are each independently N or C-L^{a}-R^{e},
at least two of Z′, Z³, and Z⁵ are N,
X² is O, S, or NR′,
L^{a}, and L³ to L⁵ are each independently a single bond, a substituted or unsubstituted C6 to C20 arylene group, a substituted or unsubstituted C2 to C20 heterocyclic group, or a combination thereof,
R^{e}, R^{f} and R²³ to R⁴⁴ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
R²³ to R³⁰ are each independently present or two adjacent ones among R²³ to R³⁰ are linked to form a substituted or unsubstituted aromatic monocyclic or polycyclic ring,
R³¹ to R³⁵ are each independently present or two adjacent ones among R³¹ to R³⁵ are linked to form a substituted or unsubstituted aromatic monocyclic or polycyclic ring,
Ar³ and Ar⁴ are each independently a substituted or unsubstituted C6 to C30 aryl group, a substituted or unsubstituted C2 to C30 heterocyclic group, or a combination thereof,
R^{a}, Ar³, and Ar⁴ are each independently present or two adjacent ones among R^{a}, Ar³, and Ar⁴ are linked to form a substituted or unsubstituted aromatic or heteroaromatic monocyclic or polycyclic ring, and
m5 and m6 are each independently one of integers of 1 to 3.

10. An organic optoelectronic device, comprising:
an anode and a cathode facing each other, and
a light emitting layer between the anode and the cathode, the light emitting layer including the compound as claimed in claim 1.

11. The organic optoelectronic device as claimed in claim 10, wherein the light emitting layer further includes a fluorescent dopant, a phosphorescent sensitizer, or a combination thereof.

12. The organic optoelectronic device as claimed in claim 11, wherein:
the phosphorescent sensitizer is an organometallic compound, and
the fluorescent dopant is a condensed polycyclic compound including bromine (B), nitrogen (N), or a combination thereof.

13. A display device comprising the organic optoelectronic device as claimed in claim 10.

14. An organic optoelectronic device, comprising:
an anode and a cathode facing each other, and
a light emitting layer between the anode and the cathode, the light emitting layer including the composition as claimed in claim 8.

15. The organic optoelectronic device as claimed in claim 14, wherein the light emitting layer further includes a fluorescent dopant, a phosphorescent sensitizer, or a combination thereof.

16. The organic optoelectronic device as claimed in claim 15, wherein:
the phosphorescent sensitizer is an organometallic compound, and
the fluorescent dopant is a condensed polycyclic compound including bromine (B), nitrogen (N), or a combination thereof.

17. A display device comprising the organic optoelectronic device as claimed in claim 14.
